(19) United States
(12) Patent Application Publication
(10) Pub. No.: US 2026/0258032 A1
(43) Pub. Date: Sep. 3, 2026

(54) TYROSINE KINASE 2 INHIBITORS AND USES THEREOF

(71) Applicant: BIOGEN MA INC., Cambridge, MA (US)

(72) Inventors: Jeffrey Vessels, Marlborough, MA (US); Lei Zhang, Westford, MA (US); Kurt D. van Vloten, Bellingham, MA (US); Simone Sciabola, Wellesley, MA (US); Harold George Vandeveer, Stoneham, MA (US); Felix Gonzalez Lopez de Turiso, San Diego, CA (US); TeYu Chen, Charlestown, MA (US); Zhili Xin, Cambridge, MA (US); Edward Yin Shiang Lin, Ashland, MA (US); Tamara Halkina Levin, Belmont, MA (US); Christopher Helal, Cambridge, MA (US); Soma Maitra, Cambridge, MA (US); Kevin M. Guckian, Northborough, MA (US)

(21) Appl. No.: 18/877,307

(22) PCT Filed: Jun. 22, 2023
(86) PCT No.: PCT/US2023/025944

§ 371 Date: Dec. 20, 2024

Related U.S. Application Data
(60) Provisional application No. 63/354,914, filed on Jun. 23, 2022.

(51) Int. Cl. C07D 471/04; A61K 31/506

(52) U.S. Cl. CPC C07D 471/04; A61K 31/506

(57) ABSTRACT

Provided are compounds of the Formula (I): or pharmaceutically acceptable salts thereof, which are useful for the inhibition of TYK2 and in the treatment of a variety of TYK2 mediated conditions or diseases.

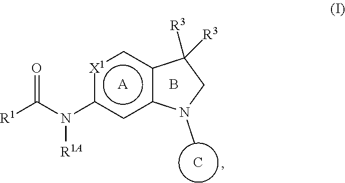

### RELATED APPLICATIONS

This application claims priority to U.S. Provisional Application No. 63/354,914 filed on Jun. 23, 2022. The entire contents of the foregoing application are expressly incorporated herein by reference.

### FIELD OF THE INVENTION

The present disclosure relates to inhibitors of Tyrosine kinase 2 (TYK2), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the TYK2 inhibitor to a warm-blooded animal, especially a human.

### BACKGROUND OF THE INVENTION

Cytokines are small secreted proteins released by cells and have a specific effect on the interactions and communications between cells. Cytokine pathways mediate a broad range of biological functions including many aspects of inflammation and immunity through mostly extracellular signaling.

Tyrosine kinase 2 (TYK2) is a member of Janus kinases (JAK) that are cytoplasmic protein kinases associated with cytokine receptors and play a central role in mediating cytokine signaling (Kisseleva et al., Gene, 2002, 285, 1; and Yamaoka et al. Genome Biology 2004, 5, 253). The JAK family also includes JAK1, JAK2 and JAK3. More specifically, cytokine's engagement with cognate receptors triggers activation of receptors associate with JAK, which leads to JAK mediated tyrosine phosphorylation of signal transducer and activator of transcription (STAT) proteins and ultimately transcriptional activation of specific gene sets (Schindler et al, 2007, J. Biol. Chem. 282: 20059-63). Numerous cytokines known to activate the JAK family include the interferon (IFN) family (IFN-alpha, IFN-beta, IFN-omega, Limitin, IFN-gamma, IL-10, IL-19, IL-20, IL-22), the glycoprotein (gp) 130 family (IL-6, IL-11, OSM, LlF, CNTF, NNT-1/BSF-3, G-CSF, CT-1, Leptin, IL-12, IL-23), the gamma C family (IL-2, IL-7, TSLP, IL-9, IL-15, IL-21, IL-4, IL-13), IL-3 family (IL-3, IL-5, GM-CSF), the single chain family (EPO, GH, PRL, TPO), receptor tyrosine kinases (EGF, PDGF, CSF-1, HGF), and G-protein coupled receptors (AT1).

TYK2 is important in the signaling of the type I interferons (e.g., IFN-alpha), IL-6, IL-10, IL-12 and IL-23 (Liang, Y. et al., Expert Opinion on Therapeutic Targets, 2014, 18, 5, 571-580; Kisseleva et al., 2002, Gene 285:1-24; and Watford, W. T. & O'Shea, J. J., 2006, Immunity 25:695-697). Consistent with this, primary cells derived from a TYK2 deficient human are defective in type I interferon, IL-6, IL-10, IL-12 and IL-23 signaling. TYK2 signals with other members of the JAK family in the following combinations: TYK2/JAK1, TYK2/JAK2, TYK2/JAK1/JAK2.

Studies have shown that inappropriate JAK activities can arise from mutation, over-expression, or inappropriate regulation, dys-regulation or de-regulation, as well as over- or under-production of growth factors or cytokines, and therefore trigger a variety of biological cellular responses relating to cell growth, cell differentiation, cell function, survival, apoptosis, and cell mobility. The inappropriate JAK activities are implicated in many diseases that include but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease.

Small molecule JAK inhibitors have emerged as a major therapeutic advancement in treating autoimmune diseases. To date, all known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents catalytic activity of the kinase by blocking ATP, downstream phosphorylation, and resulting pathway signal transduction (Bryan et al., J. Med. Chem. 2018, 61, 9030-9058).

Because of the high homology of the ATP active site across the kinome and especially within the JAK family, it is a significant challenge to achieve high selectivity for a specific JAK family member while also maintaining selectivity within the kinome. As a result, many JAK inhibitors that have been developed are pan-JAK inhibitors or are modestly selective for one or more JAK family members. While these inhibitors have shown encouraging results in treating autoimmune diseases, undesirable side effects leading to a narrow therapeutic index have been observed and suggest the need for improved treatments.

TYK2 has been shown to be important in the differentiation and function of multiple cell types important in inflammatory disease and autoimmune disease including natural killer cells, B cells, and T helper cell types. Aberrant TYK2 expression is associated with multiple autoimmune or inflammatory conditions.

There remains a need for potent compounds that demonstrate high selectivity for TYK2 over other members of the JAK family as potential therapeutic agents for treating diseases or disorders that are responsive to TYK2 inhibition.

### SUMMARY OF THE INVENTION

The present disclosure provides compounds that are TYK2 inhibitors. In a first aspect, the present disclosure relates to compounds having the Formula I:

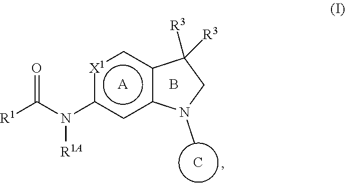

or a pharmaceutically acceptable salt thereof, wherein:
   ring A is an aromatic or heteroaromatic ring fused with ring B that is a 5-membered heterocycle;
   X¹ is N or CH;
   ring C represents is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{C};
   each R^{C} is independently halo, —CN, —NR^{N1}R^{N2}, —NR^{N3}—C(O)—R⁷, —C(O)—NR^{N3}R^{N4}, —NR^{N4}—SO₂—R⁷, —C(O)—R⁷, —C(O)—OH, —C(O)—OR⁷, —SR⁷, —SO₂—R⁷, —OR^{O1}, C₁₋₆ alkyl, C₂₋₆alkenyl, phenyl, 5 to 6 membered heteroaryl, 3 to 7 membered monocyclic carbocyclyl or 4 to 9 membered monocyclic or bicyclic heterocyclyl, wherein the C₁₋₆ alkyl, C₁₋₆alkenyl, phenyl, 5 to 6 membered heteroaryl, 3 to 7 membered monocyclic carbocyclyl and 4 to 9 membered monocyclic or bicyclic heterocyclyl represented by R^{C} are each optionally substituted with one or more R^{C1};
   each R^{C1} is independently halo, oxo, —CN, —OR^{O1}, —NR^{N3}R^{N4}, —C(O)—R⁷, —C(O)—OR^{O3}, —SO₂—R⁷, C₁₋₆ alkyl, phenyl, 3 to 7 membered monocyclic carbocyclyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N3}R^{N4}, C₁₋₆ alkyl, C₁₋₄ haloalkyl, phenyl, 5 to 6 membered heteroaryl, 3 to 7 membered monocyclic carbocyclyl, and 4 to 8 membered monocyclic heterocyclyl;
   R¹ is H, C₁₋₆ alkyl, —OR², —NR²R⁴, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R⁸;
   R^{1A} is H or C₁₋₃alkyl;
   each R² and R⁴ are independently H, C₁₋₄alkyl, or 3 to 4 membered monocyclic carbocyclyl;
   each R³ is independently H, halo, or C₁₋₃ alkyl;
   alternatively two R³, together with the atom to which they are attached, form a 3 to 7 membered monocyclic carbocyclyl or 4 to 9 membered monocyclic heterocyclyl, each of which is optionally substituted by one or more R⁵;
   each R⁵ is independently H, halo, —CN, —OR^{O1}, —NR^{N3}R^{N4}, C₁₋₃ alkyl, —C(O)—R⁷, —C(O)—OR^{O3}, —C(O)—NR^{N1}R^{N2}, —NR^{N3}—C(O)—R⁷, SO₂—R⁷, phenyl, 5 to 6 membered heteroaryl, or 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁵ are each optionally substituted by one or more substituents independently selected from halo, oxo, —CN, —OR^{O1}, —NR^{N1}R^{N2}, C₁₋₆ alkyl, C₁₋₄ haloalkyl, 3 to 7 membered monocyclic carbocyclyl and 4 to 7 membered monocyclic heterocyclyl;
   each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{N3}R^{N4}, C₁₋₆ alkyl, C₁₋₄ haloalkyl, 3 to 7 membered monocyclic carbocyclyl and 4 to 7 membered monocyclic heterocyclyl;
   each R⁸ is independently halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
   each R^{O1} is independently H, C₁₋₆ alkyl, phenyl, 5 to 6 membered heteroaryl, 3 to 7 membered monocyclic carbocyclyl, or 4 to 8 membered monocyclic or bicyclic heterocyclyl, wherein the C₁₋₆ alkyl, phenyl, 5 to 6 membered heteroaryl, 3 to 8 membered monocyclic carbocyclyl, and 4 to 7 membered monocyclic or bicyclic heterocyclyl represented by R^{O1} are each optionally substituted by one or more R^{O2};
   each R^{O2} is independently halo, OH, —CN, C₁₋₄ alkoxy, C₁₋₄alkyl, C₁₋₄alkyl-C₁₋₄alkoxy, C₁₋₄ haloalkyl, phenyl, 5 to 6 membered heteroaryl, 3 to 7 membered monocyclic carbocylyl or 4 to 7 membered monocyclic or bicyclic heterocyclyl, wherein the 4 to 7 membered monocyclic or bicyclic heterocyclyl is optionally substituted with C₁₋₄alkyl or C₁₋₄alkoxy;
   each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
   R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
   R^{N1} and R^{N2} are each independently H or C₁₋₆ alkyl, wherein the C₁₋₆ alkyl represented by R^{N1} and R^{N2} are each optionally substituted with halo, C₁₋₄alkoxy or —NR^{N3}R^{N4}, or
   R^{N1} is H or C₁₋₃alkyl and R^{N2} is ring D or —C₁₋₃alkylene-ring D;
   Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and C₁₋₄alkoxy;
   each R^{N3} is independently H or C₁₋₆ alkyl; and
   each R^{N4} is independently H or C₁₋₆ alkyl.

Another aspect of the disclosure relates to pharmaceutical compositions comprising compounds of Formula (I) or pharmaceutically acceptable salts thereof, and a pharmaceutically acceptable carrier.

In yet another aspect, the present disclosure provides a method of treating a disease or disorder that is responsive to inhibition of TYK2 in a subject comprising administering to said subject an effective amount of at least one compound described herein or a pharmaceutically acceptable salt thereof.

Another aspect of the present disclosure relates to the use of at least one compound described herein or a pharmaceutically acceptable salt thereof, for the manufacture of a medicament for the treatment of a disease or disorder responsive to inhibition of TYK2. Also provided is a compound described herein or a pharmaceutically acceptable salt thereof for use in treating a disease or disorder responsive to inhibition of TYK2. Use of a compound described herein or a pharmaceutically acceptable salt thereof for treating a disease or disorder responsive to inhibition of TYK2 is also included in the present disclosure.

### DETAILED DESCRIPTION OF THE INVENTION

The present disclosure provides compounds and pharmaceutical compositions thereof that may be useful in the treatment of diseases or disorders through mediation of TYK2. In some embodiments, the compounds of present disclosure are TYK2 inhibitors.

### Compounds and Compositions

In a first embodiment, the present disclosure provides a compound of Formula (I):

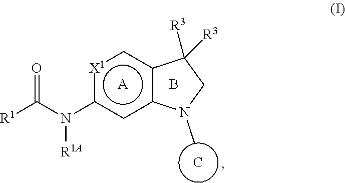

or a pharmaceutically acceptable salt thereof, wherein:
   ring A is an aromatic or heteroaromatic ring fused with ring B that is a 5-membered heterocycle;
   X¹ is N or CH;
   ring C represents is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{C};
   each R^{C} is independently halo, —CN, —NR^{N1}R^{N2}, —NR^{N3}—C(O)—R⁷, —C(O)—NR^{N3}R^{N4}, —NR^{N4}—SO₂—R⁷, —C(O)—R⁷, —SR⁷, —SO₂—R⁷, —OR^{O1}, C₁₋₆ alkyl, phenyl, 5 to 6 membered heteroaryl, 3 to 7 membered monocyclic carbocyclyl or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R^{C} are each optionally substituted with one or more R^{C1};
   each R^{C1} is independently halo, oxo, —CN, —ORO, —NR^{N3}R^{N4}, —C(O)—R⁷, —C(O)—OR^{O3}, —SO₂—R⁷, C₁₋₆ alkyl, phenyl, 3 to 7 membered monocyclic carbocyclyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N3}R^{N4}, C₁₋₆ alkyl, C₁₋₄ haloalkyl, phenyl, 5 to 6 membered heteroaryl, 3 to 7 membered monocyclic carbocyclyl, and 4 to 8 membered monocyclic heterocyclyl;
   R¹ is H, C₁₋₆ alkyl, —OR², —NR²R⁴, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R⁸;
   R^{1A} is H or C₁₋₃alkyl;
   each R² and R⁴ are independently H, C₁₋₄alkyl, or 3 to 4 membered monocyclic carbocyclyl;
   each R³ is independently H, halo, or C₁₋₃ alkyl;
   alternatively two R³, together with the atom to which they are attached, form a 3 to 7 membered monocyclic carbocyclyl or 4 to 9 membered monocyclic heterocyclyl, each of which is optionally substituted by one or more R⁵;
   each R⁵ is independently H, halo, —CN, —OR^{O1}, —NR^{N3}R^{N4}, C₁₋₃ alkyl, —C(O)—R⁷, —C(O)—OR^{O3}, —C(O)—NR^{N1}R^{N2}, —NR^{N3}—C(O)—R⁷, SO₂—R⁷, phenyl, 5 to 6 membered heteroaryl, or 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁵ are each optionally substituted by one or more substituents independently selected from halo, oxo, —CN, —OR^{O1}, —NR^{N1}R^{N2}, C₁₋₆ alkyl, C₁₋₄ haloalkyl, 3 to 7 membered monocyclic carbocyclyl and 4 to 7 membered monocyclic heterocyclyl;
   each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{N3}R^{N4}, C₁₋₆ alkyl, C₁₋₄ haloalkyl, 3 to 7 membered monocyclic carbocyclyl and 4 to 7 membered monocyclic heterocyclyl;
   each R⁸ is independently halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
   each R^{O1} is independently H, C₁₋₆ alkyl, phenyl, 5 to 6 membered heteroaryl, 3 to 7 membered monocyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic carbocyclyl, and 4 to 7 membered monocyclic or bicyclic heterocyclyl represented by R^{O1} are each optionally substituted by one or more R^{O2};
   each R^{O2} is independently halo, OH, —CN, C₁₋₄ alkoxy, C₁₋₄ haloalkyl, phenyl, 5 to 6 membered heteroaryl, 3 to 7 membered monocyclic carbocylyl or 4 to 7 membered monocyclic heterocyclyl;
   each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
   R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —N^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
   R^{N1} and R^{N2} are each independently H or C₁₋₆ alkyl, wherein the C₁₋₆ alkyl represented by R^{N1} and R^{N2} are each optionally substituted with halo, C₁₋₄alkoxy or —NR^{N3}R^{N4}, or
   R^{N1} is H or C₁₋₃alkyl and R^{N2} is ring D or —C₁₋₃alkylene-ring D;
   Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and C₁₋₄alkoxy;
   each R^{N3} is independently H or C₁₋₆ alkyl; and
   each R^{N4} is independently H or C₁₋₆ alkyl.

In a second embodiment, for the compounds of Formula (I), or a pharmaceutically acceptable salt thereof, R^{1A} is H or —CH₃; and the remaining variables are as described in the first aspect or the first embodiment.

In a third embodiment, for the compounds of Formula (I), or a pharmaceutically acceptable salt thereof, each R³ is independently H or —CH₃; and the remaining variables are as described in the first aspect or the first or second embodiment.

In a fourth embodiment, for the compounds of Formula (I), or a pharmaceutically acceptable salt thereof, two R³, together with the atom to which they are attached, form C₃₋₆ cycloalkyl or 5 to 6 membered monocyclic heterocyclyl, each of which is optionally substituted by one to three R⁵; and the remaining variables are as described in the first aspect or the first or second embodiment.

In a fifth embodiment, for the compounds of Formula (I), or a pharmaceutically acceptable salt thereof, two R³, together with the atom to which they are attached, form cyclopropane, cyclobutane, cyclopentane, tetrahydropyran, or piperidine, each of which is optionally substituted by one to three R⁵; and the remaining variables are as described in the first aspect or the first, second, or fourth embodiment.

In a sixth embodiment, for the compounds of Formula (I), or a pharmaceutically acceptable salt thereof, two R³ together with the atom to which they are attached form:

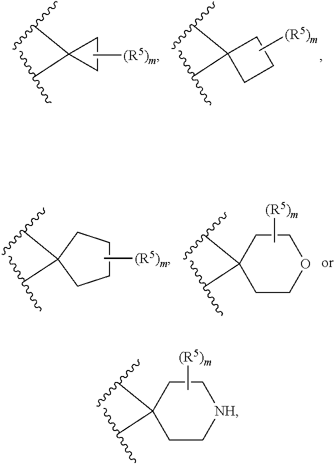

wherein m is 0, 1, 2 or 3, and [symbol] represents a bond within ring B; and the remaining variables are as described in the first aspect or the first, second, or fourth embodiment.

In a seventh embodiment, the compound of the present disclosure is represented by Formula (III), (IV), (V), (VI), (VII), (VIII) or (IX):

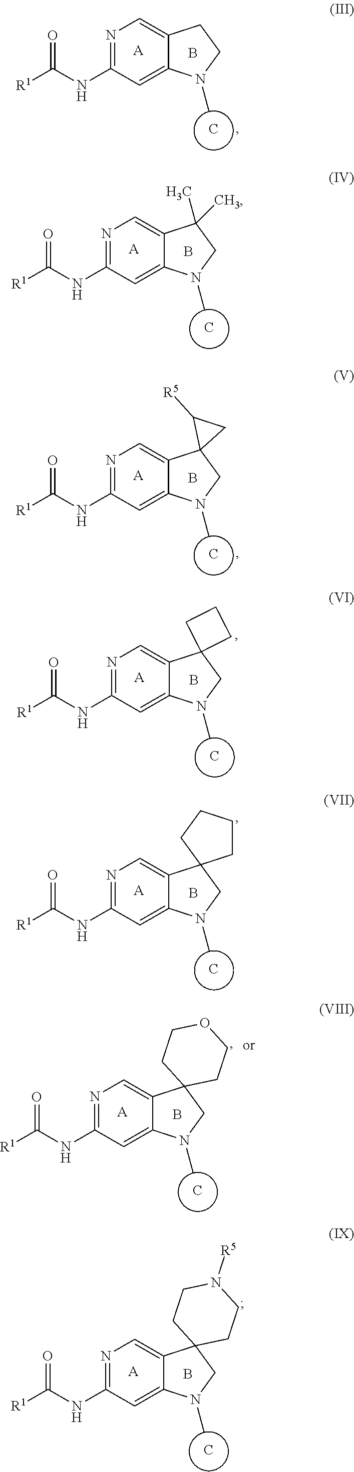

or a pharmaceutically acceptable salt thereof, wherein the variables in Formula (III), (IV), (V), (VI), (VII), (VIII) or (IX) are as defined in the first embodiment above.

In an eighth embodiment, for the compounds of Formula (I), (III), (IV), (V), (VI), (VII), (VIII) or (IX), or a pharmaceutically acceptable salt thereof, each R⁵ is independently H, —CN, or CH₃; and the remaining variables are as described in the first aspect or the first, second, fourth, fifth, sixth, or seventh embodiment.

In a ninth embodiment, for the compounds of Formula (I), (III), (IV), (V), (VI), (VII), (VIII) or (IX), or a pharmaceutically acceptable salt thereof, ring C is 5 to 9 membered monocyclic or bicyclic heteroaryl optionally substituted by one to three R^{C}; and the remaining variables are as described in the first aspect or the first, second, third, fourth, fifth, sixth, seventh, or eighth embodiment.

In a tenth embodiment, for the compounds of Formula (I), (III), (IV), (V), (VI), (VII), (VIII) or (IX), or a pharmaceutically acceptable salt thereof, ring C is pyrazine, pyrozolo[1,5-a]pyrimidine, pyridine, pyrimidine, pyrimidinone or thiazole, each of which is optionally substituted by one or three R^{C}; and the remaining variables are as described in the first aspect or the first, second, third, fourth, fifth, sixth, seventh, eighth, or ninth embodiment.

In an eleventh embodiment, for the compounds of Formula (I), (III), (IV), (V), (VI), (VII), (VIII) or (IX), or a pharmaceutically acceptable salt thereof, ring C is selected from

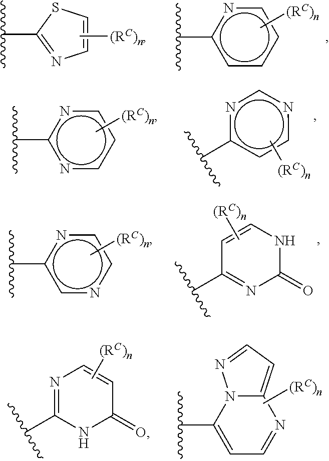

and wherein [symbol] represents a bond to ring B, and n is 0, 1, 2, or 3; and the remaining variables are as described in the first aspect or the first, second, third, fourth, fifth, sixth, seventh, eighth, or ninth embodiment.

In a twelfth embodiment, for the compounds of Formula (I), (III), (IV), (V), (VI), (VII), (VIII) or (IX), or a pharmaceutically acceptable salt thereof, ring C is selected from

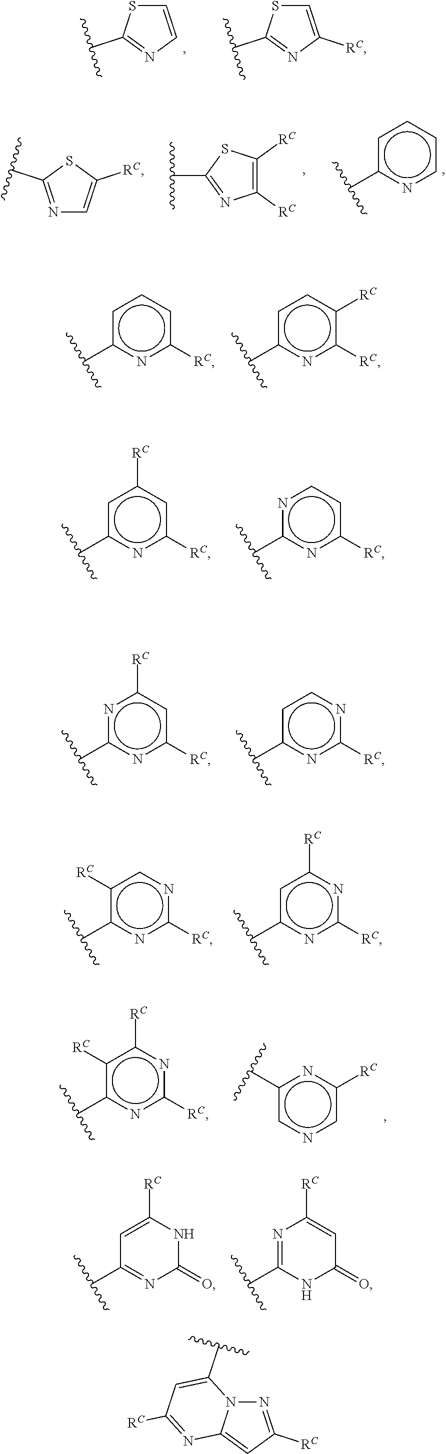

and wherein [symbol] represents a bond to ring B, and the one to three R^{C} groups in ring C may be the same or different; and the remaining variables are as described in the first aspect or the first, second, third, fourth, fifth, sixth, seventh, eighth, or ninth embodiment.

In a thirteenth embodiment, for the compounds of Formula (I), (III), (IV), (V), (VI), (VII), (VIII) or (IX), or a pharmaceutically acceptable salt thereof, each R^{C} is independently halo, —NR^{N1}R^{N2}, —OR^{O1}, —SR⁷, —SO₂—R⁷, —C(O)—NR^{N3}R^{N4}, —C(O)—R⁷, C₁₋₃ alkyl, C₃₋₆ cycloalkyl, 4 to 9 membered monocyclic or bicyclic heterocyclyl, phenyl or 5 to 6 membered heteroaryl, wherein the C₁₋₃ alkyl, C₃₋₆ cycloalkyl, 4 to 9 membered monocyclic or bicyclic heterocyclyl, phenyl and 5 to 6 membered heteroaryl represented by R^{C} are each optionally substituted with one to three R^{C1};
R⁷ is C₁₋₃alkyl;
R^{O1} is H, C₁₋₄alkyl, C₁₋₄alkyl, C₃₋₆cycloalkyl, 4 to 6 membered monocyclic heterocyclyl, 5 to 6 membered heteroaryl, wherein C₁₋₄alkyl represented by R^{O1} is optionally substituted with one to three substituents independently selected from halo, CN, C₁₋₃alkoxy, C₃₋₆cycloalkyl, 4 to 6 membered monocyclic heterocyclyl, and wherein C₃₋₆cycloalkyl, 4 to 6 membered monocyclic heterocyclyl, and 5 to 6 membered heteroaryl represented by R^{O1} are each optionally substituted with one to two substitutents independently selected from halo, C₁₋₃alkyl, CN, OH and C₁₋₃alkoxy;
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and —C₁₋₄alkoxy; R^{N1} and R^{N2} are each independently H or C₁₋₄ alkyl, wherein the C₁₋₄ alkyl represented by R^{N1} and R^{N2} are each optionally substituted with halo, C₁₋₃alkoxy or —NR^{N3}R^{N4}, or
R^{N1} is H or C₁₋₂alkyl and R^{N2} is ring D or —C₁₋₂alkylene-ring D;
Ring D is 3 to 6 membered monocyclic or bicyclic carbocyclyl, or 4 to 6 membered monocyclic heterocyclyl, wherein the 3 to 6 membered monocyclic carbocyclyl and 4 to 6 membered monocyclic heterocyclyl represented by ring D are each optionally substituted with one or two substituents independently selected from halo, C₁₋₃ alkyl and C₁₋₃alkoxy;
each R^{N3} is independently H or C₁₋₃alkyl;
each R^{N4} is independently H or C₁₋₃alkyl; and the remaining variables are as described in the first aspect or the first, second, third, fourth, fifth, sixth, seventh, eighth, ninth, tenth, eleventh, or twelfth embodiment.

Alternatively, as part of the thirteenth embodiment, for the compounds of Formula (I), (III), (IV), (V), (VI), (VII), (VIII) or (IX), or a pharmaceutically acceptable salt thereof, each R^{C} is independently halo, —NR^{N1}R^{N2}, —OR^{O1}, —SR⁷, —SO₂—R⁷, —C(O)—NR^{N3}R^{N4}, —C(O)—R⁷, —C(O)H, —C(O)OH, —C(O)OR⁷, C₁₋₆ alkyl, C₂₋₄alkenyl, C₃₋₆ cycloalkyl, 4 to 9 membered monocyclic or bicyclic heterocyclyl, phenyl or 5 to 6 membered heteroaryl, wherein the C₁₋₆ alkyl, C₂₋₄alkenyl, C₃₋₆ cycloalkyl, 4 to 9 membered monocyclic or bicyclic heterocyclyl, phenyl and 5 to 6 membered heteroaryl represented by R^{C} are each optionally substituted with one to three R^{C1};
R⁷ is C₁₋₃alkyl;
R^{O1} is H, C₁₋₄alkyl, C₁₋₄alkyl, C₃₋₆cycloalkyl, 4 to 8 membered monocyclic or bicyclic heterocyclyl, 5 to 6 membered heteroaryl, wherein C₁₋₄alkyl represented by R^{O1} is optionally substituted with one to three substituents independently selected from halo, OH, CN, C₁₋₃alkoxy, C₃₋₆cycloalkyl, and 4 to 6 membered monocyclic heterocyclyl optionally substituted with C₁₋₃alkyl or C₁₋₃alkoxy, and wherein C₃₋₆cycloalkyl, 4 to 8 membered monocyclic or bicyclic heterocyclyl, and 5 to 6 membered heteroaryl represented by R^{O1} are each optionally substituted with one to two substitutents independently selected from halo, C₁₋₃alkyl, C₁₋₃alkyl-C₁₋₁₃alkoxy, CN, OH and C₁₋₃alkoxy;
R^{N1} and R^{N2} are each independently H or C₁₋₆ alkyl, wherein the C₁₋₆ alkyl represented by R^{N1} and R^{N2} are each optionally substituted with halo, C₁₋₄alkoxy or —NR^{N3}R^{N4}, or
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl, and —C₁₋₄alkoxy; R^{N1} and R^{N2} are each independently H or C₁₋₄ alkyl, wherein the C₁₋₄ alkyl represented by R^{N1} and R^{N2} are each optionally substituted with halo, C₁₋₃alkoxy or —NR^{N3}R^{N4}, or
each R^{N3} is independently H or C₁₋₃alkyl; and
each R^{N4} is independently H or C₁₋₃alkyl; and the remaining variables are as described in the first aspect or the first, second, third, fourth, fifth, sixth, seventh, eighth, ninth, tenth, eleventh, or twelfth embodiment.

In a fourteenth embodiment, for the compounds of Formula (I), (III), (IV), (V), (VI), (VII), (VIII) or (IX), or a pharmaceutically acceptable salt thereof, ring D is cyclobutane, cyclopropane, bicyclo[1.1.1]pentane, spiro[2.2]pentane, azetidine, oxetane, tetrahydrofuran, tetrahydropyran, tetrahydrothiophene or thietane, each of which is optionally substituted with one or two substituents independently selected from halo, C₁₋₂alkyl and C₁₋₂alkoxy; and the remaining variables are as described in the first aspect or the first, second, third, fourth, fifth, sixth, seventh, eighth, ninth, tenth, eleventh, twelfth, or thirteenth embodiment.

In a fifteenth embodiment, for the compounds of Formula (I), (III), (IV), (V), (VI), (VII), (VIII) or (IX), or a pharmaceutically acceptable salt thereof, ring D is

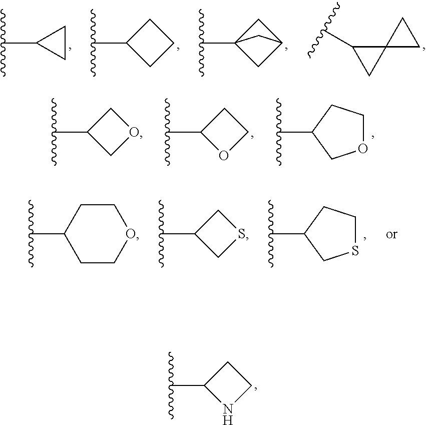

each of which is optionally substituted with one or two substituents independently selected from F, —CH₃ and —OCH₃; and the remaining variables are as described in the first aspect or the first, second, third, fourth, fifth, sixth, seventh, eighth, ninth, tenth, eleventh, twelfth, thirteenth, or fourteenth embodiment.

In a sixteenth embodiment, for the compounds of Formula (I), (III), (IV), (V), (VI), (VII), (VIII) or (IX), or a pharmaceutically acceptable salt thereof, the 4 to 6 membered monocyclic heterocyclyl represented by R^{O1} or a substituent of C₁₋₄alkyl represented by R^{O1} is oxetane, azetindine or tetrahydrofuran, each of which is optionally substituted with one to two substituents independently selected with C₁₋₃alkyl, halo, CN, OH or C₁₋₃alkoxy; and the 5 to 6 membered heteroaryl represented by R^{O1} is pyrazole, triazole, pyridine, pyrazine, pyrimidine, or pyridazine, each of which is optionally substituted with one to two substituents independently selected with C₁₋₃alkyl, halo, CN, OH or C₁₋₃alkoxy; and the remaining variables are as described in the first aspect or the first, second, third, fourth, fifth, sixth, seventh, eighth, ninth, tenth, eleventh, twelfth, or thirteenth embodiment. Alternatively, as part of the sixteenth embodiment, for the compounds of Formula (I), (III), (IV), (V), (VI), (VII), (VIII) or (IX), or a pharmaceutically acceptable salt thereof, the 4 to 8 membered monocyclic or bicyclic heterocyclyl represented by R^{O1} or a substituent of C₁₋₄alkyl represented by R^{O1} is oxetane, azetindine, pyrrolidine, 2-oxaspiro[3.3]heptanyl, or tetrahydrofuran, each of which is optionally substituted with one to two substituents independently selected with C₁₋₃alkyl, halo, CN, OH, C₁₋₃alkyl-C₁₋₃alkoxy, or C₁₋₃alkoxy; and the 5 to 6 membered heteroaryl represented by R^{O1} is pyrazole, triazole, pyridine, pyrazine, pyrimidine, or pyridazine, each of which is optionally substituted with one to two substituents independently selected with C₁₋₃alkyl, halo, CN, OH or C₁₋₃alkoxy; and the remaining variables are as described in the first aspect or the first, second, third, fourth, fifth, sixth, seventh, eighth, ninth, tenth, eleventh, twelfth, or thirteenth embodiment.

In a seventeenth embodiment, for the compounds of Formula (I), (III), (IV), (V), (VI), (VII), (VIII) or (IX), or a pharmaceutically acceptable salt thereof, the 4 to 6 membered monocyclic heterocyclyl is

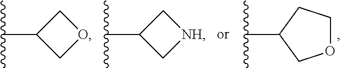

each of which is optionally substituted with one or two substituents independently selected from F, —CN, —CH₃, —CH₂CH₃, OH and —OCH₃;
the 5 to 6 membered heteroaryl is:

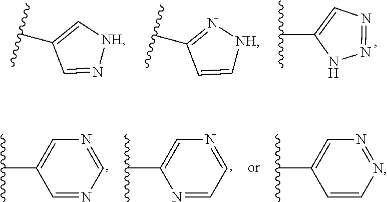

each of which is optionally substituted with one or two substituents independently selected from F, —CN, —CH₃, —CH₂CH₃, OH and —OCH₃; and the remaining variables are as described in the first aspect or the first, second, third, fourth, fifth, sixth, seventh, eighth, ninth, tenth, eleventh, twelfth, thirteenth, or sixteenth embodiment.

Alternatively, as part of the seventeenth embodiment, for the compounds of Formula (I), (III), (IV), (V), (VI), (VII), (VIII) or (IX), or a pharmaceutically acceptable salt thereof, the 4 to 6 membered monocyclic heterocyclyl is:

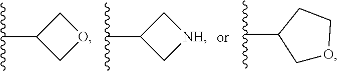

each of which is optionally substituted with one or two substituents independently selected from F, —CN, —CH₃, —CH₂CH₃, OH, —CH₂OCH₃, and —OCH₃; and
the 5 to 6 membered heteroaryl is:

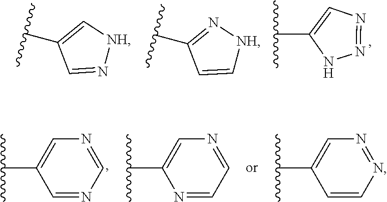

each of which is optionally substituted with one or two substituents independently selected from F, —CN, —CH₃, —CH₂CH₃, OH and —OCH₃; and the remaining variables are as described in the first aspect or the first, second, third, fourth, fifth, sixth, seventh, eighth, ninth, tenth, eleventh, twelfth, thirteenth, or sixteenth embodiment.

In an eighteenth embodiment, for the compounds of Formula (I), (III), (IV), (V), (VI), (VII), (VIII) or (IX), or a pharmaceutically acceptable salt thereof, each R^{c} is independently selected from F, —CH₃, —CHF₂, —CF₃, —CH₂CH₃, —CH₂CH₂CH₃, —CH₂CH₂CH₂CH₃, —CF₂CH₃, —CF₂CFH₂, —CFHCFH₂, —CH(CH₃)₂, —CF(CH₃)₂, —C(CH₃)₃, —CH₂OH, —CH₂OCH₃, —CH₂CH₂CH₂OCH₃, —CH(CH₃)OCH₃, —C(CH₃)₂OCH₃, —CH₂OCH₂CH₃, —CH₂-cyclopropyl, —CH(CH₃)—SO₂CH₃, OH, —OCH₃, —OCH₂CH₂OCH₃, —OCH₂CH₂CH₂OCH₃, —OCH₂CH₂N(CH₃)₂, —OCH(CH₃)CH₂OCH₃, —OCH(CH₃)CN, —C(O)NH₂, —C(O)NHCH₃, —C(O)N(CH₃)₂, —C(O)H, —C(O)CH₃, —NH₂, —NHCH₃, —NHCH₂CH₃, —NHCH₂CH₂CH₃, —NHCH(CH₃)₂, —N(CH₃)₂, —N(CH₃)CH(CH₃)₂, —NHCH₂CH₂CH₂F, —NHCH₂CHF₂, —NHCH₂C(F)CH₃, —NHCH₂CH₂OCH₃, —NHCH₂CH(CH₃)N(CH₃)₂, —NHCH₂C(CH₃)₂OCH₃, —NHCH₂CH₂C(F)CH₃, —NHCH₂CH(CH₃)OCH₃, —NHCH(CH₃)CH(CH₃)OCH₃, —N(CH₃)CH₂CH₂OCH₃, —NHC(O)CH₃, —SCH₃, —SO₂CH₃,

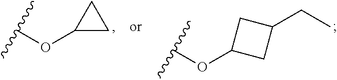

and the remaining variables are as described in the first aspect or the first, second, third, fourth, fifth, sixth, seventh, eighth, ninth, tenth, eleventh, twelfth, or thirteenth embodiment. Alternatively, as part of the eighteenth embodiment, for the compounds of Formula (I), (III), (IV), (V), (VI), (VII), (VIII) or (IX), or a pharmaceutically acceptable salt thereof, each R^{C} is independently selected from F, —Cl, —CH₃, —CHF₂, —CF₃, —CH₂CH₃, —CH₂CH₂CH₃, —CH₂CH₂CH₂CH₃, —CH₂CH₂CH₂CH₂CH₃, —CF₂CH₃, —CF₂CFH₂, —CFHCFH₂, —CF₂CH₂CH₃, —CF₂CH₂OH, —CF₂CH₂OCH₃, —CH(CH₃)₂, —CF(CH₃)₂, —C(CH₃)₃, —CF₂C(O)OCH₂CH₃, —CH₂OH, —CH(OH)CH₃, —CH₂OCH₃, —CH₂CH₂CH₂OH, —CH₂CH₂OCH₂CH₃, —CH₂CH₂CH₂OCH₃, —CH₂CH₂C(CH₃)(OH)CH₂CH₃, —CH₂CH₂CH₂OC(CH₃)₃, —CH₂CH₂C(CH₃)₂OH, —CH(CH₃)OCH₃, —C(CH₃)₂OCH₃, —CH₂OCH₂CH₃, —CH₂-cyclopropyl, —CH(CH₃)—SO₂CH₃, —CH₂CH₂—SO₂CH₃, —CH₂NHCH₃, —CH₂CN, —CH(CH₃)CN, —CH═CH—OCH₂CH₃, OH, —OCH₃, —OCH₂CH₃, —OCH(CH₃)₂, —OCH₂CF₃, —OCH₂CF₂CH₃, —OCH₂CH₂OCH₃, —OCH₂CH₂F, —OCH₂CH₂CH₂OCH₃, —OCH₂CH₂N(CH₃)₂, —OCH(CH₃)CH₂OCH₃, —OCH(CH₃)CN, —OCH₂C(CH₃)₂OH, —OCH₂C(CH₃)₂OCH₃, —OCH(cyclopropyl)(CN), —C(O)NH₂, —C(O)NHCH₃, —C(O)N(CH₃)₂, —C(O)H, —C(O)CH₃, —C(O)OH, —C(O)OCH₂CH₃, —NH₂, —NHCH₃, —NHCH₂CH₃, —NHCH₂CH₂CH₃, —NHCH(CH₃)₂, —N(CH₃)₂, —N(CH₃)CH(CH₃)₂, —NHCH₂CH₂CH₂F, —NHCH₂CHF₂, —NHCH₂C(F)CH₃, —NHCH₂CH₂OCH₃, —NHCH(CH₃)CH₂OCH₃, —NHCH₂CH(CH₃)N(CH₃)₂, —NHCH₂C(CH₃)₂OCH₃, —NHCH₂CH₂C(F)CH₃, —NHCH₂CH(CH₃)OCH₃, —NHCH(CH₃)CH(CH₃)OCH₃, —N(CH₃)CH₂CH₂OCH₃, —NHC(O)CH₃, —SCH₃, —SO₂CH₃,

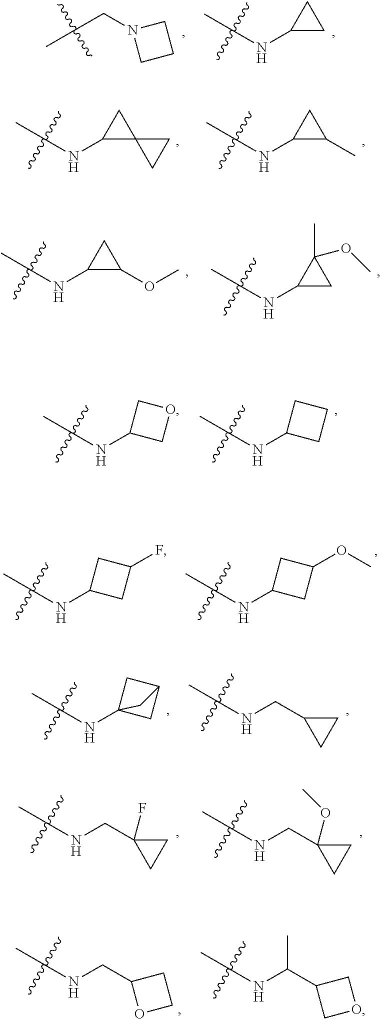

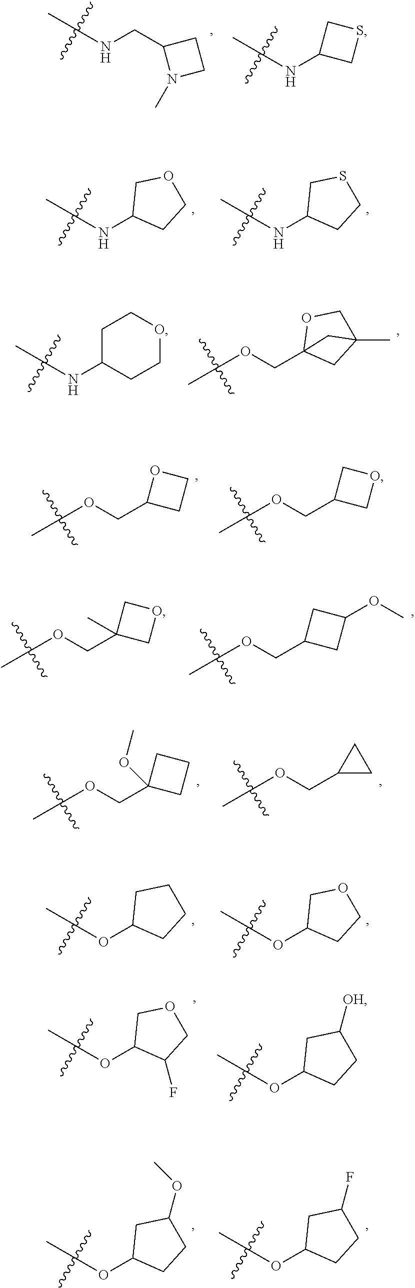

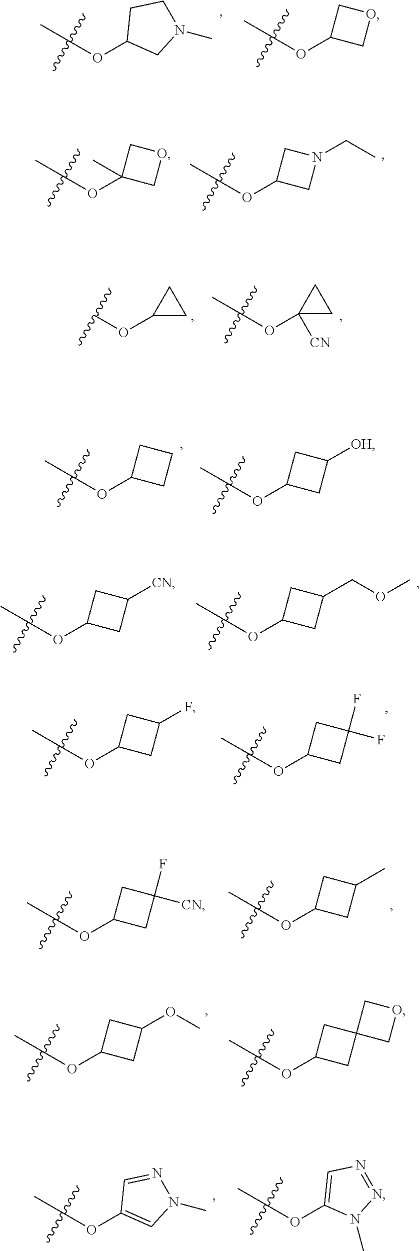

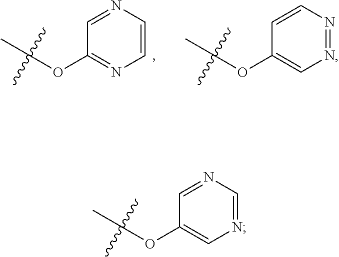 [SMILES: *Oc1cnccn1 ; *Oc1ccnnc1 ; *Oc1cncnc1]

and the remaining variables are as described in the first aspect or the first, second, third, fourth, fifth, sixth, seventh, eighth, ninth, tenth, eleventh, twelfth, or thirteenth embodiment.

In a nineteenth embodiment, for the compounds of Formula (I), (III), (IV), (V), (VI), (VII), (VIII) or (IX), or a pharmaceutically acceptable salt thereof, each C₃₋₆cycloalkyl represented by R^{C} is selected from cyclobutane, cyclopentane and cyclopropane;
each 4 to 9 membered monocyclic or bicyclic heterocyclyl represented by R^{C} is independently selected from azetidine, morpholine, octahydropyrrolo[3,4-b]pyrrole, octahydropyrrolo[3,4-c]pyrrole, oxaazaspiro[3.5]nonane, oxabicyclo[2.2.1]heptane, oxetane, piperidine, piperazine, pyrrolidine, tetrahydrofuran, tetrahydrothiophene dioxide and tetrahydropyran;
each of the 5 to 6 membered heteroaryl represented by R^{C} is independently selected from oxazole, pyrazole, pyridine, pyrimidine and thiazole;
wherein each of the C₃₋₆ cycloalkyl, 4 to 9 membered monocyclic or bicyclic heterocyclyl and 5 to 6 membered heteroaryl is optionally substituted with one to three R^{C1}; and the remaining variables are as described in the first aspect or the first, second, third, fourth, fifth, sixth, seventh, eighth, ninth, tenth, eleventh, twelfth, or thirteenth embodiment.

Alternatively, as part of the nineteenth embodiment, for the compounds of Formula (I), (III), (IV), (V), (VI), (VII), (VIII) or (IX), or a pharmaceutically acceptable salt thereof, each C₃₋₆cycloalkyl represented by R^{C} is selected from cyclobutane, cyclopentane and cyclopropane;
each 4 to 9 membered monocyclic or bicyclic heterocyclyl represented by R^{C} is independently selected from azetidine, morpholine, octahydropyrrolo[3,4-b]pyrrole, octahydropyrrolo[3,4-c]pyrrole, oxaazaspiro[3.5]nonane, oxabicyclo[2.1.1]hexane, oxabicyclo[2.2.1]heptane, oxetane, piperidine, piperazine, pyrrolidine, diydrofuran, tetrahydrofuran, tetrahydrothiophene dioxide and tetrahydropyran; and
each of the 5 to 6 membered heteroaryl represented by R^{C} is independently selected from oxazole, pyrazole, pyridine, pyrimidine, triazole, and thiazole;
and wherein each of the C₃₋₆ cycloalkyl, 4 to 9 membered monocyclic or bicyclic heterocyclyl and 5 to 6 membered heteroaryl is optionally substituted with one to three R^{C1}; and the remaining variables are as described in the first aspect or the first, second, third, fourth, fifth, sixth, seventh, eighth, ninth, tenth, eleventh, twelfth, or thirteenth embodiment.

In a twentieth embodiment, for the compounds of Formula (I), (III), (IV), (V), (VI), (VII), (VIII) or (IX), or a pharmaceutically acceptable salt thereof, each of the C₃₋₆cycloalkyl represented by R^{C} is independently selected from

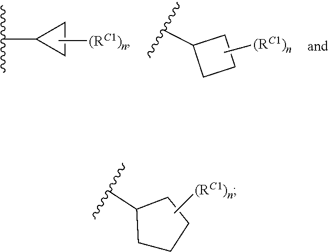

each of the 4 to 9 membered monocyclic heterocyclyl represented by R^{C} is independently selected from

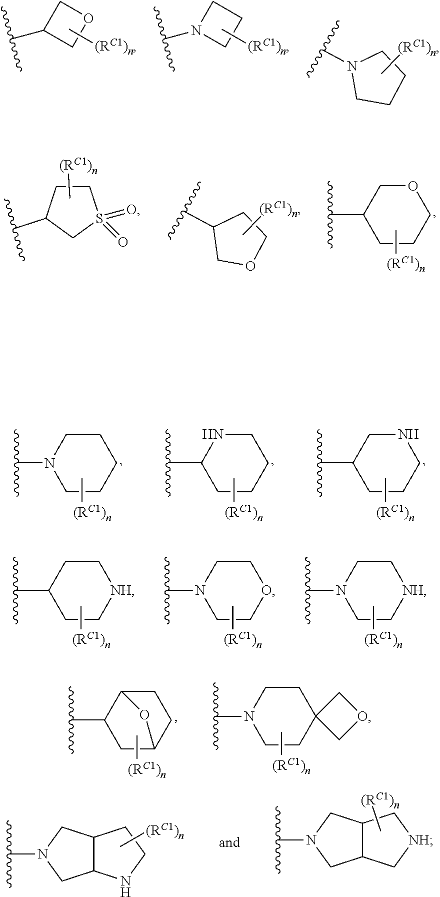

each of the phenyl represented by R^{C} is:

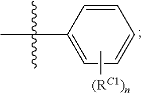

each of the 5 to 6 membered heteroaryl represented by R^{C} is independently selected

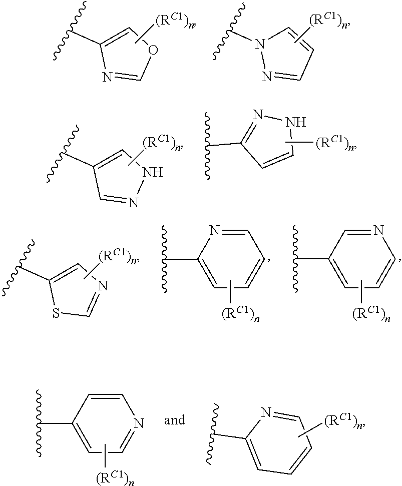

wherein [symbol] represents a bond to ring C, and n is 0, 1, 2 or 3 as valency permits; and the remaining variables are as described in the first aspect or the first, second, third, fourth, fifth, sixth, seventh, eighth, ninth, tenth, eleventh, twelfth, or thirteenth embodiment.

Alternatively, as part of the twentieth embodiment, for the compounds of Formula (I), (III), (IV), (V), (VI), (VII), (VIII) or (IX), or a pharmaceutically acceptable salt thereof, each of the C₃₋₆cycloalkyl represented by R^{C} is independently selected from

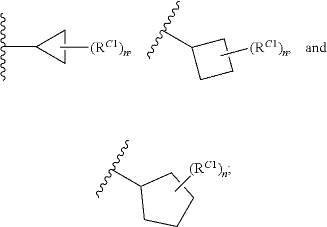

each of the 4 to 9 membered monocyclic heterocyclyl represented by R^{C} is independently selected from

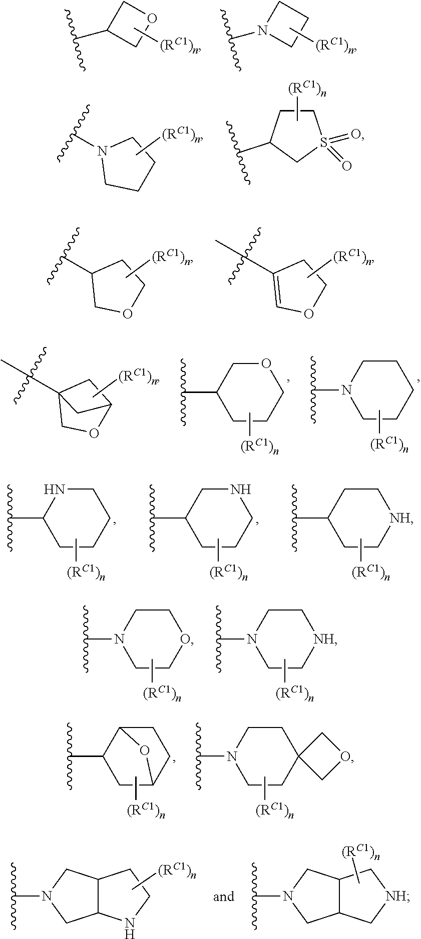

each of the phenyl represented by R^{C} is:

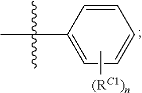

and
each of the 5 to 6 membered heteroaryl represented by R^{C} is independently selected from

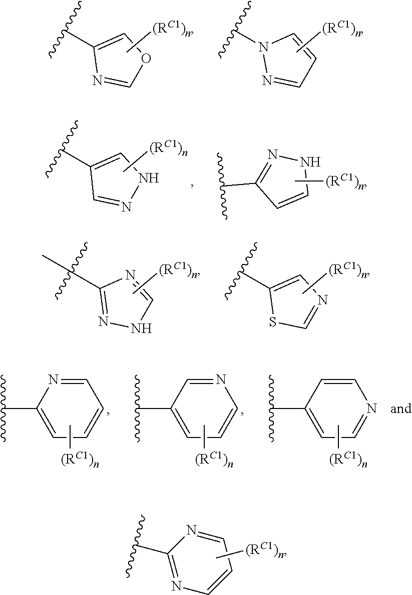

and wherein [symbol] represents a bond to ring C, and n is 0, 1, 2 or 3 as valency permits; and the remaining variables are as described in the first aspect or the first, second, third, fourth, fifth, sixth, seventh, eighth, ninth, tenth, eleventh, twelfth, or thirteenth embodiment.

In a twenty-first embodiment, for the compounds of Formula (I), (III), (IV), (V), (VI), (VII), (VIII) or (IX), or a pharmaceutically acceptable salt thereof, each of the C₃₋₆ cycloalkyl represented by R^{C} is independently selected from:

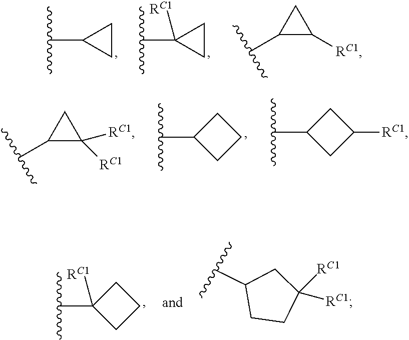

each of the 4 to 6 membered monocyclic heterocyclyl represented by R^{C} is independently selected from:

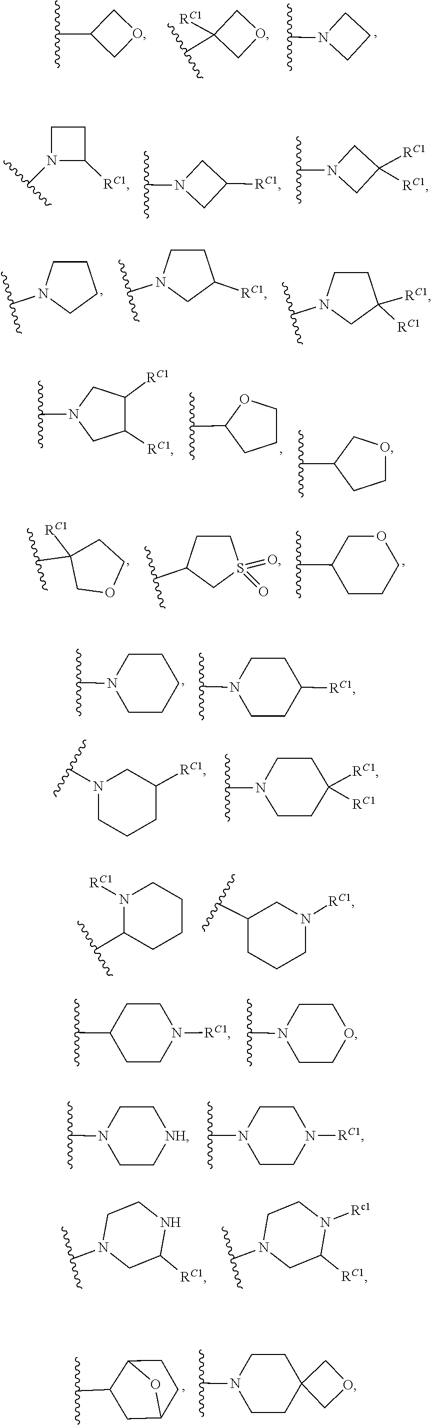

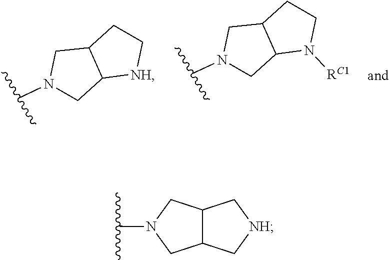

and
each of the phenyl represented by R^{C} is independently selected from and

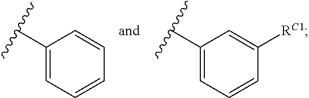

each of the 5 to 6 membered heteroaryl represented by R^{C} is independently selected from

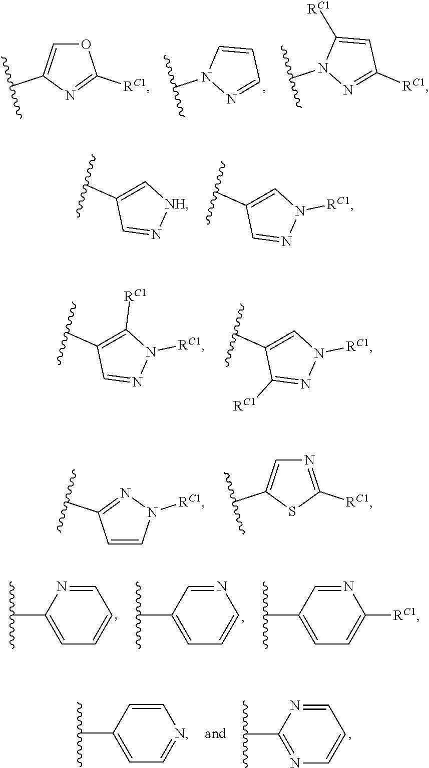

wherein [symbol] represents a bond ring C; and the remaining variables are as described in the first aspect or the first, second, third, fourth, fifth, sixth, seventh, eighth, ninth, tenth, eleventh, twelfth, or thirteenth embodiment.

Alternatively, as part of the twenty-first embodiment, for the compounds of Formula (I), (III), (IV), (V), (VI), (VII), (VIII) or (IX), or a pharmaceutically acceptable salt thereof, each of the C₃₋₆ cycloalkyl represented by R^{C} is independently selected from:

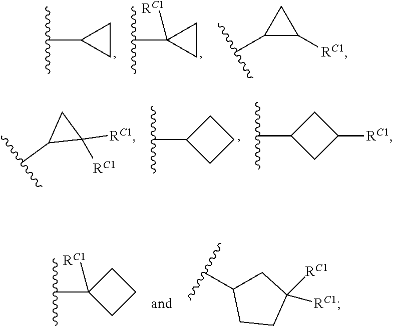

each of the 4 to 6 membered monocyclic heterocyclyl represented by R^{C} is independently selected from:

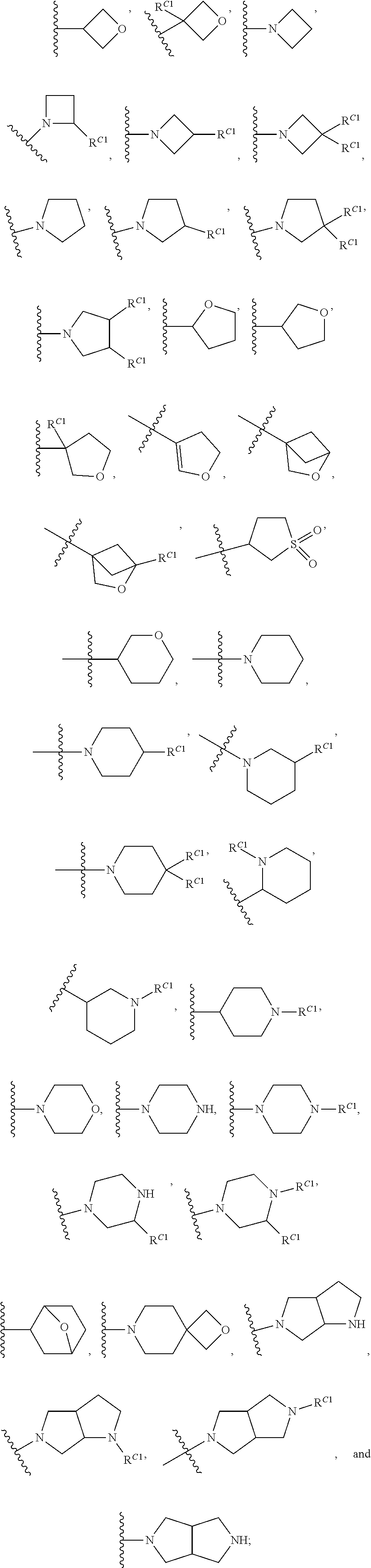

and
each of the phenyl represented by R^{C} is independently selected from

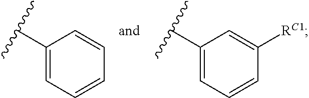

each of the 5 to 6 membered heteroaryl represented by R^{C} is independently selected from

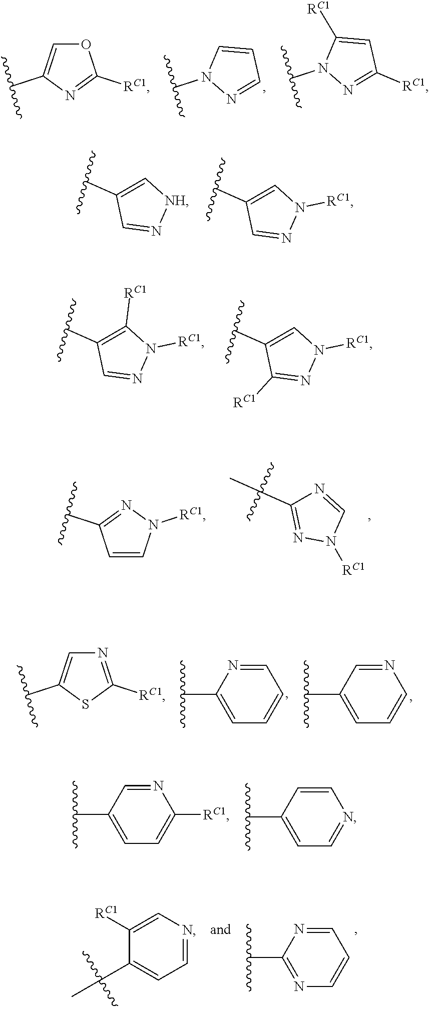

wherein [symbol] represents a bond ring C; and the remaining variables are as described in the first aspect or the first, second, third, fourth, fifth, sixth, seventh, eighth, ninth, tenth, eleventh, twelfth, or thirteenth embodiment.

In a twenty-second embodiment, for the compounds of Formula (I), (III), (IV), (V), (VI), (VII), (VIII) or (IX), or a pharmaceutically acceptable salt thereof, each R^{C1} is independently selected from halo, OH, —NR^{N3}R^{N4}, —CN, —C(O)—R⁷, —SO₂—R⁷, C₁₋₃alkoxy, C₁₋₃ haloalkoxy, C₃₋₆cycloalkyl, 4 to 6 membered monocyclic heterocyclyl, and C₁₋₄ alkyl, wherein the C₁₋₄alkyl represented by R^{C1} is optionally substituted by one to three substituents independently selected from C₃₋₅cycloalkyl, C₁₋₃alkoxy, OH and halo, and the C₁₋₃alkoxy represented by R^{C1} is optionally substituted with one to three substituents independently selected from C₁₋₃alkoxy and halo; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment. Alternatively, as part of the In a twenty-second embodiment, for the compounds of Formula (I), (III), (IV), (V), (VI), (VII), (VIII) or (IX), or a pharmaceutically acceptable salt thereof, each R^{C1} is independently selected from halo, OH, —NR^{N3}R^{N4}, —CN, —C(O)—R⁷, —SO₂—R⁷, C₁₋₃ alkoxy, C₁₋₃ haloalkoxy, C₃₋₆ cycloalkyl, 4 to 6 membered monocyclic heterocyclyl, and C₁₋₄ alkyl, wherein the C₁₋₄alkyl represented by R^{C1} is optionally substituted by one to three substituents independently selected from C₃₋₅ cycloalkyl, 4 to 8 membered monocyclic heterocyclyl, C₁₋₃alkoxy, OH, CN, —NR^{N3}R^{N4} and halo, and the C₁₋₃alkoxy represented by R^{C1} is optionally substituted with one to three substituents independently selected from C₁₋₃alkoxy and halo; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment.

In a twenty-third embodiment, for the compounds of Formula (I), (III), (IV), (V), (VI), (VII), (VIII) or (IX), or a pharmaceutically acceptable salt thereof, each R^{C1} is independently selected from F, —CN, —C(O)CH₃, —C(O)-cyclopropyl, —SO₂—CH₃, —SO₂—CH₂CH₃, OH, —OCH₃, —OCH₂CH₂OCH₃, —OCHF₂, —N(CH₃)₂, —CH₃, —CH₂CH₃, —CH(CH₃)₂, —CH₂CH₂OH, —CH₂CH₂OCH₃, —CH₂-cyclopropyl, —CH₂F, —CHF₂, —CF₃, cyclobutyl, cyclopropyl,

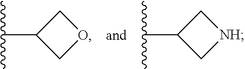 [SMILES: *C1COC1 ; *C1CNC1]

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment. Alternatively, as part of the twenty-third embodiment, for the compounds of Formula (I), (III), (IV), (V), (VI), (VII), (VIII) or (IX), or a pharmaceutically acceptable salt thereof, each R^{C1} is independently selected from F, —CN, —C(O)CH₃, —C(O)-cyclopropyl, —SO₂—CH₃, —SO₂—CH₂CH₃, OH, —OCH₃, —OCH₂CH₂OCH₃, —OCHF₂, —N(CH₃)₂, —CH₂CH₂N(CH₃)₂, —CH₃, —CH₂CH₃, —CH(CH₃)₂, —CH₂CH(CH₃)₂, —CH₂CH₂OH, —CH₂CH₂OCH₃, —CH₂OCH₃, —CH₂-cyclopropyl, —CH₂F, —CHF₂, —CF₃, —CH₂CF₃, —CF(CH₃)₂, —C(OH)(CH₃)₂, —CH₂CN, —CHFCH₃, —CF₂CH₂OCH₃, cyclobutyl, cyclopropyl,

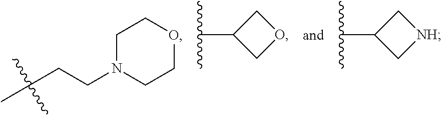

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fourth embodiment, for the compounds of Formula (I), (III), (IV), (V), (VI), (VII), (VIII) or (IX), or a pharmaceutically acceptable salt thereof, R¹ is C₁₋₄ alkyl optionally substituted by one to three R⁸ independently selected from halo, —CN, C₁₋₃alkoxy, C₁₋₃alkyl and C₁₋₃haloalkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment. Alternatively, as part of the twenty-fourth embodiment, for the compounds of Formula (I), (III), (IV), (V), (VI), (VII), (VIII) or (IX), or a pharmaceutically acceptable salt thereof, R¹ is C₁₋₄ alkyl optionally substituted by one to three R⁸ independently selected from halo, —CN, —NR²R⁴, C₁₋₃alkoxy, C₁₋₃alkyl and C₁₋₃ haloalkyl, and R² and R⁴ are each independently H or C₁₋₃alkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment, for the compounds of Formula (I), (III), (IV), (V), (VI), (VII), (VIII) or (IX), or a pharmaceutically acceptable salt thereof, R¹ is —CH₃, —CH₂CH₃, or —CH₂CH₂OCH₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment. Alternatively, as part of the twenty-fifth embodiment, for the compounds of Formula (I), (III), (IV), (V), (VI), (VII), (VIII) or (IX), or a pharmaceutically acceptable salt thereof, R¹ is —CH₃, —CH₂CH₃, —NH₂, —NHCH₃, —N(CH₃)₂, —OCH₃, or —CH₂CH₂OCH₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-sixth embodiment, for the compounds of Formula (I), (III), (IV), (V), (VI), (VII), (VIII) or (IX), or a pharmaceutically acceptable salt thereof, each R^{O1} is independently H, C₁₋₃ alkyl or C₃₋₆cycloalkyl, wherein the C₁₋₃ alkyl and C₃₋₆cycloalkyl represented by R^{O1} are optionally substituted by C₁₋₃ alkoxy; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, or twenty-fifth embodiment.

In a twenty-seventh embodiment, for the compounds of Formula (I), (III), (IV), (V), (VI), (VII), (VIII) or (IX), or a pharmaceutically acceptable salt thereof, each R^{O1} is independently H, —CH₃, —CH₂—CH₂—OCH₃,

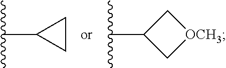

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-eighth embodiment, for the compounds of Formula (I), (III), (IV), (V), (VI), (VII), (VIII) or (IX), or a pharmaceutically acceptable salt thereof, R^{N1} and R^{N2} each independently represent H or C₁₋₃alkyl optionally substituted with C₁₋₃alkoxy; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, or twenty-seventh embodiment.

In a twenty-ninth embodiment, for the compounds of Formula (I), (III), (IV), (V), (VI), (VII), (VIII) or (IX), or a pharmaceutically acceptable salt thereof, R^{N1} and R^{N2} each independently represent H, —CH₃ or —CH₂—CH₂—OCH₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment, the compound of the present disclosure is represented by Formula (X) or (XI):

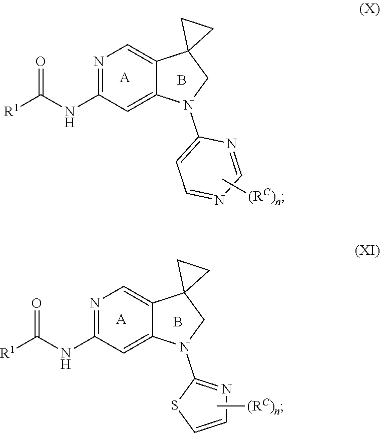

or a pharmaceutically acceptable salt thereof, wherein:
n is 1, 2 or 3;
each R^{C} is independently —NR^{N1}R^{N2}, —OR^{O1}, or C₁₋₆ alkyl, 4 to 6 membered monocyclic heterocyclyl, wherein the C₁₋₆ alkyl and 4 to 6 membered monocyclic heterocyclyl represented by R^{C} are each optionally substituted with one to three R^{C1};
each R^{C1} is independently halo;
R¹ is C₁₋₃ alkyl;
R^{O1} is C₃₋₄cycloalkyl optionally substituted by R^{O2};
R^{O2} is C₁₋₃ alkoxy; and
R^{N1} and R^{N2} are each independently H or C₁₋₃ alkyl optionally substituted with C₁₋₃ alkoxy.

In a thirty-first embodiment, the compound of the present disclosure is represented by Formula (XA), (XIA), (XIB), (XIC) or (XID):

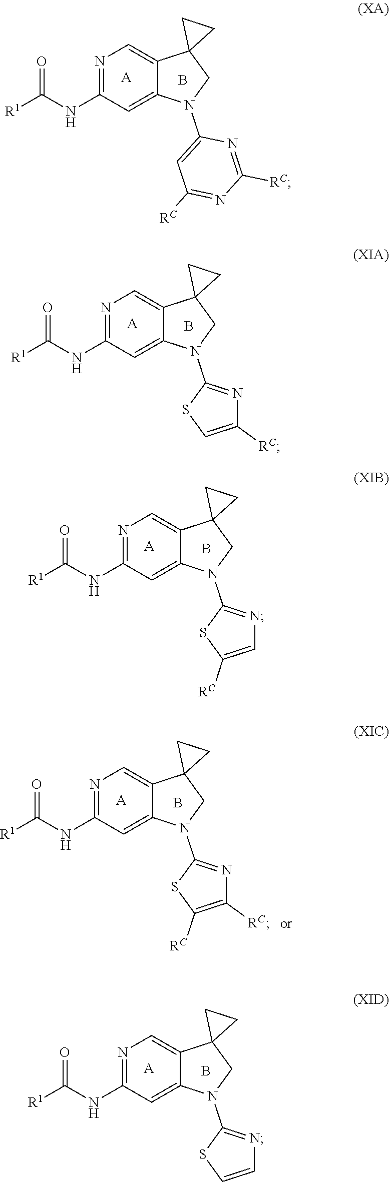

or a pharmaceutically acceptable salt thereof, wherein the variables in Formula (III), (IV), (V), (VI), (VII), (VIII) or (IX) are as defined in the thirtieth embodiment above.

In a thirty-second embodiment, for the compounds of Formula (X), (XI), (XA), (XIA), (XIB), (XIC) or (XID) or a pharmaceutically acceptable salt thereof, each R^{C} is independently —NHCH₃, —NHCH₂CH₂OCH₃, —CH₃, —CH₂CH₃, —CH(CH₃)₂, —CF₂CH₃, —CF(CH₃)₂,

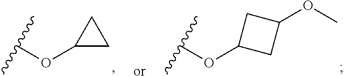

R¹ is CH₃; and the remaining variables are as described in the thirtieth or thirty-first embodiment.

In a thirty-third embodiment, for the compounds of Formula (X), (XI), (XA), (XIA), (XIB), (XIC) or (XID) or a pharmaceutically acceptable salt thereof, R^{C} is 4 to 6 membered monocyclic heterocyclyl independently selected from tetrahydrofuran and tetrahydropyran, each of which is optionally substituted with one to three R^{C1}; and the remaining variables are as described in the thirtieth or thirty-first embodiment.

In a thirty-fourth embodiment, for the compounds of Formula (X), (XI), (XA), (XIA), (XIB), (XIC) or (XID) or a pharmaceutically acceptable salt thereof, R^{C} is independently selected from

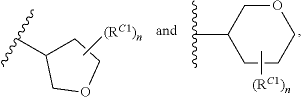

wherein [symbol] represents a bond to ring C; and the remaining variables are as described in the thirtieth or thirty-first embodiment.

In a thirty-fifth embodiment, for the compounds of Formula (X), (XI), (XA), (XIA), (XIB), (XIC) or (XID) or a pharmaceutically acceptable salt thereof, R^{C} is independently selected from

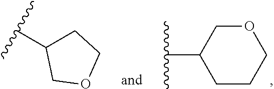

wherein [symbol] represents a bond to ring C; and the remaining variables are as described in the thirtieth or thirty-first embodiment.

In a thirty-sixth embodiment, for the compounds of Formula (X), (XI), (XA), (XIA), (XIB), (XIC) or (XID) or a pharmaceutically acceptable salt thereof, each R^{C1} is F; and the remaining variables are as described in the thirtieth, thirty-first, thirty-second, thirty-third, or thirty-fourth embodiment.

In another embodiment, the compound of the present disclosure is represented by Formula (XII):

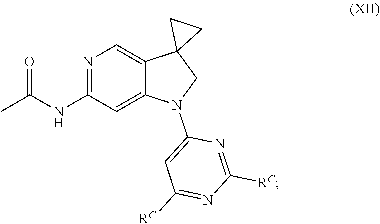

or a pharmaceutically acceptable salt thereof, wherein:
   each R^{C} is independently C₁₋₃alkyl, C₁₋₃haloalkyl, or —NR^{N1}R^{N2};
   R^{N1} and R^{N2} are each independently H or C₁₋₃alky; and the remaining variables are as described in the first aspect or first embodiment above.

In some embodiments, for the compounds of Formula (XII), each R^{C} is, independently, —CH₃, —CH₂CH₃, —CF₂CH₃, or —NHCH₃; and the remaining variables are as described above for the compounds of Formula (XII).

In a thirty-seventh embodiment, the present disclosure provides a compound described herein (e.g. a compound of any one of Examples 1 to 411), or a pharmaceutically acceptable salt thereof.

In a thirty-eighth embodiment, the present disclosure provides a compound selected from the group consisting of:
N-(1′-(2-(1,1-difluoroethyl)-6-methoxypyrimidin-4-yl)-1′,2′-dihydrospiro[cyclopropane-1,3′-pyrrolo[3,2-c]pyridin]-6′-yl)acetamide;
N-(1′-(4-(1,1-difluoroethyl)-6-methoxypyrimidin-2-yl)-1′,2′-dihydrospiro[cyclopropane-1,3′-pyrrolo[3,2-c]pyridin]-6′-yl)acetamide;
N-(1′-(2-(oxetan-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(2-methoxyethoxy)-3,6-dihydropyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(3-methoxycyclobutoxy)pyrimidin-4-yl)-1′,2′-dihydrospiro[cyclopropane-1,3′-pyrrolo[3,2-c]pyridin]-6′-yl)acetamide;
N-(1′-(6-cyclopropoxy-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-(1,1-difluoroethyl)-5-fluoropyridin-2-yl)-1′,2′-dihydrospiro[cyclopropane-1,3′-pyrrolo[3,2-c]pyridin]-6′-yl)acetamide;
N-(1′-(6-(1,1-difluoroethyl)-5-methoxypyridin-2-yl)-1′,2′-dihydrospiro[cyclopropane-1,3′-pyrrolo[3,2-c]pyridin]-6′-yl)acetamide;
N-(1′-(6-(1,1-difluoroethyl)-4-methoxypyridin-2-yl)-1′,2′-dihydrospiro[cyclopropane-1,3′-pyrrolo[3,2-c]pyridin]-6′-yl)acetamide;
N-(1′-(2-(1,1-difluoroethyl)-6-(methylamino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-ethylpyrimidin-4-yl)-1′,2′-dihydrospiro[cyclopropane-1,3′-pyrrolo[3,2-c]pyridin]-6′-yl)acetamide;
N-(1′-(6-(1,1-difluoroethyl)pyridin-2-yl)-1′,2′-dihydrospiro[cyclopropane-1,3′-pyrrolo[3,2-c]pyridin]-6′-yl)acetamide;
N-(1′-(2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(4-(1,1-difluoroethyl)pyrimidin-2-yl)-1′,2′-dihydrospiro[cyclopropane-1,3′-pyrrolo[3,2-c]pyridin]-6′-yl)acetamide;
N-(1′-(6-acetamido-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-(tetrahydrofuran-3-yl)pyridin-2-yl)-1′,2′-dihydrospiro[cyclopropane-1,3′-pyrrolo[3,2-c]pyridin]-6′-yl)acetamide;
N-(1′-(2-(2-fluoropropan-2-yl)pyrimidin-4-yl)-1′,2′-dihydrospiro[cyclopropane-1,3′-pyrrolo[3,2-c]pyridin]-6′-yl)acetamide;
N-(1′-(6-(3-methoxytetrahydrofuran-3-yl)pyridin-2-yl)-1′,2′-dihydrospiro[cyclopropane-1,3′-pyrrolo[3,2-c]pyridin]-6′-yl)acetamide;
N-(1′-(6-(3-hydroxytetrahydrofuran-3-yl)pyridin-2-yl)-1′,2′-dihydrospiro[cyclopropane-1,3′-pyrrolo[3,2-c]pyridin]-6′-yl)acetamide;
N-(1′-(2-(1,1-difluoroethyl)-6-(dimethylamino)pyrimidin-4-yl)-1′,2′-dihydrospiro[cyclopropane-1,3′-pyrrolo[3,2-c]pyridin]-6′-yl)acetamide;
N-(1′-(6-cyclopropyl-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(oxetan-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(3-methoxycyclobutyl)pyrimidin-4-yl)-1′,2′-dihydrospiro[cyclopropane-1,3′-pyrrolo[3,2-c]pyridin]-6′-yl)acetamide;
N-(1′-(6-cyclobutyl-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(tetrahydrofuran-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(tetrahydrofuran-2-yl)pyrimidin-4-yl)-1′,2′-dihydrospiro[cyclopropane-1,3′-pyrrolo[3,2-c]pyridin]-6′-yl)acetamide;
N-(1′-(6-methylpyrimidin-4-yl)-1′,2′-dihydrospiro[cyclopropane-1,3′-pyrrolo[3,2-c]pyridin]-6′-yl)acetamide;
N-(1′-(2,5-dimethylpyrazolo[1,5-a]pyrimidin-7-yl)-1′,2′-dihydrospiro[cyclopropane-1,3′-pyrrolo[3,2-c]pyridin]-6′-yl)acetamide;
N-(1′-(6-methyl-2-(methylthio)pyrimidin-4-yl)-1′,2′-dihydrospiro[cyclopropane-1,3′-pyrrolo[3,2-c]pyridin]-6′-yl)acetamide;
N-(1-(2-(1,1-difluoroethyl)pyrimidin-4-yl)-2,3-dihydro-1H-pyrrolo[3,2-c]pyridin-6-yl)acetamide;
N-(1-(4-(1,1-difluoroethyl)pyrimidin-2-yl)-2,3-dihydro-1H-pyrrolo[3,2-c]pyridin-6-yl)acetamide;
N-(1-(6-(1,1-difluoroethyl)pyridin-2-yl)-2,3-dihydro-1H-pyrrolo[3,2-c]pyridin-6-yl)acetamide;
N-(1-(6-(tetrahydrofuran-3-yl)pyridin-2-yl)-2,3-dihydro-1H-pyrrolo[3,2-c]pyridin-6-yl)acetamide;
N-(1-(2-(1,1-difluoroethyl)-6-methoxypyrimidin-4-yl)-2,3-dihydro-1H-pyrrolo[3,2-c]pyridin-6-yl)acetamide;
N-(1-(2-(1,1-difluoroethyl)-6-(methylamino)pyrimidin-4-yl)-2,3-dihydro-1H-pyrrolo[3,2-c]pyridin-6-yl)acetamide;
N-(1′-(4-(1,1-difluoroethyl)pyrimidin-2-yl)-2-methyl-1′,2′-dihydrospiro[cyclopropane-1,3′-pyrrolo[3,2-c]pyridin]-6′-yl)acetamide;
N-(1′-(2-(1,1-difluoroethyl)pyrimidin-4-yl)-2-methyl-1′,2′-dihydrospiro[cyclopropane-1,3′-pyrrolo[3,2-c]pyridin]-6′-yl)acetamide;
N-(1-(2-(1,1-difluoroethyl)pyrimidin-4-yl)-3,3-dimethyl-2,3-dihydro-1H-pyrrolo[3,2-c]pyridin-6-yl)acetamide;
N-(1-(4-(1,1-difluoroethyl)pyrimidin-2-yl)-3,3-dimethyl-2,3-dihydro-1H-pyrrolo[3,2-c]pyridin-6-yl)acetamide;
N-(1′-(4-(1,1-difluoroethyl)pyrimidin-2-yl)-1′,2′-dihydrospiro[cyclobutane-1,3′-pyrrolo[3,2-c]pyridin]-6′-yl)acetamide;
N-(1′-(2-(1,1-difluoroethyl)pyrimidin-4-yl)-1′,2′-dihydrospiro[cyclobutane-1,3′-pyrrolo[3,2-c]pyridin]-6′-yl)acetamide;
N-(1′-(6-(1,1-difluoroethyl)pyridin-2-yl)-1′,2′-dihydrospiro[cyclobutane-1,3′-pyrrolo[3,2-c]pyridin]-6′-yl)acetamide;
N-(1′-(4-(1,1-difluoroethyl)pyrimidin-2-yl)-1′,2′-dihydrospiro[cyclopentane-1,3′-pyrrolo[3,2-c]pyridin]-6′-yl)acetamide;
N-(1′-(2-(1,1-difluoroethyl)pyrimidin-4-yl)-1′,2′-dihydrospiro[cyclopentane-1,3′-pyrrolo[3,2-c]pyridin]-6′-yl)acetamide;
N-(1′-(6-(1,1-difluoroethyl)pyridin-2-yl)-1′,2′-dihydrospiro[cyclopentane-1,3′-pyrrolo[3,2-c]pyridin]-6′-yl)acetamide;
N-(1′-(4-(1,1-difluoroethyl)pyrimidin-2-yl)-1′,2,2′,3,5,6-hexahydrospiro[pyran-4,3′-pyrrolo[3,2-c]pyridin]-6′-yl)acetamide;
N-(1′-(2-(1,1-difluoroethyl)pyrimidin-4-yl)-1′,2,2′,3,5,6-hexahydrospiro[pyran-4,3′-pyrrolo[3,2-c]pyridin]-6′-yl)acetamide;
N-(1′-(6-(1,1-difluoroethyl)pyridin-2-yl)-1′,2,2′,3,5,6-hexahydrospiro[pyran-4,3′-pyrrolo[3,2-c]pyridin]-6′-yl)acetamide;
N-(1′-(2-(1,1-difluoroethyl)-6-((2-methoxyethyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methoxyethyl)(methyl)amino)pyrimidin-4-yl)-1′,2′-dihydrospiro[cyclopropane-1,3′-pyrrolo[3,2-c]pyridin]-6′-yl)acetamide;
N-(1′-(6-(azetidin-1-yl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-morpholinopyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-isopropylpyrimidin-4-yl)-1′,2′-dihydrospiro[cyclopropane-1,3′-pyrrolo[3,2-c]pyridin]-6′-yl)acetamide;
N-(1′-(2-cyclopropyl-6-methylpyrimidin-4-yl)-1′,2′-dihydrospiro[cyclopropane-1,3′-pyrrolo[3,2-c]pyridin]-6′-yl)acetamide;
N-(1′-(2-(tert-butyl)-6-methylpyrimidin-4-yl)-1′,2′-dihydrospiro[cyclopropane-1,3′-pyrrolo[3,2-c]pyridin]-6′-yl)acetamide;
N-(1′-(2-methoxy-6-methylpyrimidin-4-yl)-1′,2′-dihydrospiro[cyclopropane-1,3′-pyrrolo[3,2-c]pyridin]-6′-yl)acetamide;
N-(1′-(2-(methoxymethyl)-6-methylpyrimidin-4-yl)-1′,2′-dihydrospiro[cyclopropane-1,3′-pyrrolo[3,2-c]pyridin]-6′-yl)acetamide;
N-(1′-(2-butyl-6-methylpyrimidin-4-yl)-1′,2′-dihydrospiro[cyclopropane-1,3′-pyrrolo[3,2-c]pyridin]-6′-yl)acetamide;
N-(1′-(2-(ethoxymethyl)-6-methylpyrimidin-4-yl)-1′,2′-dihydrospiro[cyclopropane-1,3′-pyrrolo[3,2-c]pyridin]-6′-yl)acetamide;
N-(1′-(2-cyclobutyl-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(trifluoromethyl)pyrimidin-4-yl)-1′,2′-dihydrospiro[cyclopropane-1,3′-pyrrolo[3,2-c]pyridin]-6′-yl)acetamide;
N-(1′-(2-(cyclopropylmethyl)-6-methylpyrimidin-4-yl)-1′,2′-dihydrospiro[cyclopropane-1,3′-pyrrolo[3,2-c]pyridin]-6′-yl)acetamide;
N-(1′-(2-(difluoromethyl)-6-methylpyrimidin-4-yl)-1′,2′-dihydrospiro[cyclopropane-1,3′-pyrrolo[3,2-c]pyridin]-6′-yl)acetamide;
N-(1′-(2-ethyl-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-propylpyrimidin-4-yl)-1′,2′-dihydrospiro[cyclopropane-1,3′-pyrrolo[3,2-c]pyridin]-6′-yl)acetamide;
N-(1′-(2-isopropyl-6-methylpyrimidin-4-yl)-1′,2′-dihydrospiro[cyclopropane-1,3′-pyrrolo[3,2-c]pyridin]-6′-yl)acetamide;
N-(1′-(5-fluoro-2,6-dimethylpyrimidin-4-yl)-1′,2′-dihydrospiro[cyclopropane-1,3′-pyrrolo[3,2-c]pyridin]-6′-yl)acetamide;
N-(1′-(2,6-dimethylpyrimidin-4-yl)-1′,2′-dihydrospiro[cyclopropane-1,3′-pyrrolo[3,2-c]pyridin]-6′-yl)acetamide;
N-(1′-(6-methyl-2-(1-methylcyclopropyl)pyrimidin-4-yl)-1′,2′-dihydrospiro[cyclopropane-1,3′-pyrrolo[3,2-c]pyridin]-6′-yl)acetamide;
N-(1′-(6-methyl-2-(tetrahydro-2H-pyran-3-yl)pyrimidin-4-yl)-1′,2′-dihydrospiro[cyclopropane-1,3′-pyrrolo[3,2-c]pyridin]-6′-yl)acetamide;
N-(1′-(2-(3-methoxypropyl)-6-methylpyrimidin-4-yl)-1′,2′-dihydrospiro[cyclopropane-1,3′-pyrrolo[3,2-c]pyridin]-6′-yl)acetamide;
trans-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide;
cis-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-(3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-((1s,3s)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-((1r,3r)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1-(6-(1,1-difluoroethyl)pyridin-2-yl)-3,3-dimethyl-2,3-dihydro-1H-pyrrolo[3,2-c]pyridin-6-yl)acetamide;
N-(1′-(thiazol-2-yl)-1′,2′-dihydrospiro[cyclopropane-1,3′-pyrrolo[3,2-c]pyridin]-6′-yl)acetamide;
N-(1′-(5-methylthiazol-2-yl)-1′,2′-dihydrospiro[cyclopropane-1,3′-pyrrolo[3,2-c]pyridin]-6′-yl)acetamide;
N-(1′-(4-(1,1-difluoroethyl)thiazol-2-yl)-1′,2′-dihydrospiro[cyclopropane-1,3′-pyrrolo[3,2-c]pyridin]-6′-yl)acetamide;
N-(1′-(4,5-dimethylthiazol-2-yl)-1′,2′-dihydrospiro[cyclopropane-1,3′-pyrrolo[3,2-c]pyridin]-6′-yl)acetamide;
N-(1′-(4-(tetrahydro-2H-pyran-3-yl)thiazol-2-yl)-1′,2′-dihydrospiro[cyclopropane-1,3′-pyrrolo[3,2-c]pyridin]-6′-yl)acetamide;
N-(1′-(5-methyl-4-(tetrahydrofuran-3-yl)thiazol-2-yl)-1′,2′-dihydrospiro[cyclopropane-1,3′-pyrrolo[3,2-c]pyridin]-6′-yl)acetamide;
N-(1′-(4-(1,1-difluoroethyl)-6-methylpyrimidin-2-yl)-1′,2′-dihydrospiro[cyclopropane-1,3′-pyrrolo[3,2-c]pyridin]-6′-yl)acetamide;
N-(1′-(6-(oxetan-3-yl)pyridin-2-yl)-1′,2′-dihydrospiro[cyclopropane-1,3′-pyrrolo[3,2-c]pyridin]-6′-yl)acetamide;
N-(1′-(6-(3-fluorooxetan-3-yl)pyridin-2-yl)-1′,2′-dihydrospiro[cyclopropane-1,3′-pyrrolo[3,2-c]pyridin]-6′-yl)acetamide;
N-(1′-(6-(1,1-difluoroethyl)-4-(2-methoxyethoxy)pyridin-2-yl)-1′,2′-dihydrospiro[cyclopropane-1,3′-pyrrolo[3,2-c]pyridin]-6′-yl)acetamide;
N-(1′-(6-(3-methoxyoxetan-3-yl)pyridin-2-yl)-1′,2′,5′,7a′-tetrahydrospiro[cyclopropane-1,3′-pyrrolo[3,2-c]pyridin]-6′-yl)acetamide;
N-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)pyrimidin-4-yl)-1′,2′-dihydrospiro[cyclopropane-1,3′-pyrrolo[3,2-c]pyridin]-6′-yl)propionamide;
N-(1′-(6-(1,1-difluoroethyl)pyridin-2-yl)-1′,2′-dihydrospiro[cyclopropane-1,3′-pyrrolo[3,2-c]pyridin]-6′-yl)propionamide;
N-(1′-(2-(1,1-difluoroethyl)pyrimidin-4-yl)-1′,2′-dihydrospiro[cyclopropane-1,3′-pyrrolo[3,2-c]pyridin]-6′-yl)-3-methoxypropanamide;
N-(1′-(6-(1,1-difluoroethyl)-4-((1r,3r)-3-methoxycyclobutoxy)pyridin-2-yl)-1′,2′,4′,7′-tetrahydrospiro[cyclopropane-1,3′-pyrrolo[3,2-c]pyridin]-6′-yl)acetamide;
N-(1′-(6-(methylsulfonyl)pyridin-2-yl)-1′,2′-dihydrospiro[cyclopropane-1,3′-pyrrolo[3,2-c]pyridin]-6′-yl)acetamide;
N-(1′-(pyridin-2-yl)-1′,2′-dihydrospiro[cyclopropane-1,3′-pyrrolo[3,2-c]pyridin]-6′-yl)acetamide;
N-(1′-(6-(3-hydroxytetrahydrofuran-3-yl)-4-methylpyridin-2-yl)-1′,2′-dihydrospiro[cyclopropane-1,3′-pyrrolo[3,2-c]pyridin]-6′-yl)acetamide;
N-(1′-(6-(3-methoxytetrahydrofuran-3-yl)-4-methylpyridin-2-yl)-1′,2′-dihydrospiro[cyclopropane-1,3′-pyrrolo[3,2-c]pyridin]-6′-yl)acetamide;
N-(1′-(6-methyl-2-(1-(methylsulfonyl)ethyl)pyrimidin-4-yl)-1′,2′-dihydrospiro[cyclopropane-1,3′-pyrrolo[3,2-c]pyridin]-6′-yl)acetamide;
N-(1′-(2-(1,1-dioxidotetrahydrothiophen-3-yl)-6-methylpyrimidin-4-yl)-1′,2′-dihydrospiro[cyclopropane-1,3′-pyrrolo[3,2-c]pyridin]-6′-yl)acetamide;
N-(1′-(4-(2-methoxyethoxy)pyrimidin-2-yl)-1′,2′-dihydrospiro[cyclopropane-1,3′-pyrrolo[3,2-c]pyridin]-6′-yl)acetamide;
N-(1′-(6-methyl-2-(1H-pyrazol-1-yl)pyrimidin-4-yl)-1′,2′-dihydrospiro[cyclopropane-1,3′-pyrrolo[3,2-c]pyridin]-6′-yl)acetamide;
N-(1′-(6-(1,1-difluoroethyl)pyrazin-2-yl)-1′,2′-dihydrospiro[cyclopropane-1,3′-pyrrolo[3,2-c]pyridin]-6′-yl)acetamide;
N-(1′-(2-(7-oxabicyclo[2.2.1]heptan-2-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(tetrahydrofuran-3-yl)pyrimidin-4-yl)-1′,2′-dihydrospiro[cyclopropane-1,3′-pyrrolo[3,2-c]pyridin]-6′-yl)acetamide;
N-(1′-(2-(1-methoxycyclobutyl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-phenylpyrimidin-4-yl)-1′,2′-dihydrospiro[cyclopropane-1,3′-pyrrolo[3,2-c]pyridin]-6′-yl)acetamide;
N-(1′-(6-methyl-2-(pyridin-3-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(pyridin-4-yl)pyrimidin-4-yl)-1′,2′-dihydrospiro[cyclopropane-1,3′-pyrrolo[3,2-c]pyridin]-6′-yl)acetamide;
N-(1′-(6-methyl-[2,2′-bipyrimidin]-4-yl)-1′,2′-dihydrospiro[cyclopropane-1,3′-pyrrolo[3,2-c]pyridin]-6′-yl)acetamide;
N-(1′-(6-methyl-2-(2-methyloxazol-4-yl)pyrimidin-4-yl)-1′,2′-dihydrospiro[cyclopropane-1,3′-pyrrolo[3,2-c]pyridin]-6′-yl)acetamide;
N-(1′-(2-(3,5-dimethyl-1H-pyrazol-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-fluorophenyl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(pyridin-2-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(piperidin-1-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(pyrrolidin-1-yl)pyrimidin-4-yl)-1′,2′-dihydrospiro[cyclopropane-1,3′-pyrrolo[3,2-c]pyridin]-6′-yl)acetamide;
N-(1′-(2-(isopropyl(methyl)amino)-6-methylpyrimidin-4-yl)-1′,2′-dihydrospiro[cyclopropane-1,3′-pyrrolo[3,2-c]pyridin]-6′-yl)acetamide;
N-(1′-(2-(dimethylamino)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(1-methylpiperidin-3-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(1-methylpiperidin-2-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(1-(methylsulfonyl)piperidin-3-yl)pyrimidin-4-yl)-1′,2′-dihydrospiro[cyclopropane-1,3′-pyrrolo[3,2-c]pyridin]-6′-yl)acetamide;
N-(1′-(2-(1-acetylpiperidin-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1-isopropylpiperidin-4-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(1-(methylsulfonyl)piperidin-4-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(2-methylcyclopropyl)pyrimidin-4-yl)-1′,2′-dihydrospiro[cyclopropane-1,3′-pyrrolo[3,2-c]pyridin]-6′-yl)acetamide;
N-(1′-(2-(2,2-dimethylcyclopropyl)-6-methylpyrimidin-4-yl)-1′,2′-dihydrospiro[cyclopropane-1,3′-pyrrolo[3,2-c]pyridin]-6′-yl)acetamide;
N-(1′-(2-(3,3-difluorocyclopentyl)-6-methylpyrimidin-4-yl)-1′,2′-dihydrospiro[cyclopropane-1,3′-pyrrolo[3,2-c]pyridin]-6′-yl)acetamide;
N-(1′-(2-(hydroxymethyl)-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-5-fluoro-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-5-fluoropyrimidin-4-yl)-1′,2′-dihydrospiro[cyclopropane-1,3′-pyrrolo[3,2-c]pyridin]-6′-yl)acetamide;
N-(1′-(2-(2-methoxypropan-2-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1-methoxyethyl)-6-methylpyrimidin-4-yl)-1′,2′-dihydrospiro[cyclopropane-1,3′-pyrrolo[3,2-c]pyridin]-6′-yl)acetamide;
N-(1′-(2-(1-methoxyethyl)pyrimidin-4-yl)-1′,2′-dihydrospiro[cyclopropane-1,3′-pyrrolo[3,2-c]pyridin]-6′-yl)acetamide;
N-(1′-(2-(2-methoxypropan-2-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-hydroxypyrimidin-4-yl)-1′,2′-dihydrospiro[cyclopropane-1,3′-pyrrolo[3,2-c]pyridin]-6′-yl)acetamide;
N-(1′-(6-methyl-2-oxo-1,2-dihydropyrimidin-4-yl)-1′,2′-dihydrospiro[cyclopropane-1,3′-pyrrolo[3,2-c]pyridin]-6′-yl)acetamide;
N-(1′-(4-methyl-6-oxo-1,6-dihydropyrimidin-2-yl)-1′,2′-dihydrospiro[cyclopropane-1,3′-pyrrolo[3,2-c]pyridin]-6′-yl)acetamide;
N-(1′-(6-methyl-2-(methylsulfonyl)pyrimidin-4-yl)-1′,2′-dihydrospiro[cyclopropane-1,3′-pyrrolo[3,2-c]pyridin]-6′-yl)acetamide;
N-(1′-(2-(3-methoxypyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-cyclobutoxy-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((3-methyloxetan-3-yl)methoxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(2-(dimethylamino)ethoxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(oxetan-3-yloxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(pyrimidin-5-yloxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-methyl-1H-pyrazol-3-yl)oxy)pyrimidin-4-yl)-1′,2′-dihydrospiro[cyclopropane-1,3′-pyrrolo[3,2-c]pyridin]-6′-yl)acetamide;
N-(1′-(6-(1-cyanocyclopropoxy)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-methyl-1H-pyrazol-4-yl)oxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(pyrazin-2-yloxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(pyridazin-4-yloxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-ethylazetidin-3-yl)oxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-methyl-1H-1,2,3-triazol-5-yl)oxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((4-fluorotetrahydrofuran-3-yl)oxy)pyrimidin-4-yl)-1′,2′-dihydrospiro[cyclopropane-1,3′-pyrrolo[3,2-c]pyridin]-6′-yl)acetamide;
N-(1′-(6-(1-cyanoethoxy)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-(cyano(cyclopropyl)methoxy)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(isopropylamino)pyrimidin-4-yl)-1′,2′-dihydrospiro[cyclopropane-1,3′-pyrrolo[3,2-c]pyridin]-6′-yl)acetamide;
N-(1′-(6-((cyclopropylmethyl)amino)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((tetrahydro-2H-pyran-4-yl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(propylamino)pyrimidin-4-yl)-1′,2′-dihydrospiro[cyclopropane-1,3′-pyrrolo[3,2-c]pyridin]-6′-yl)acetamide;
N-(1′-(6-(cyclobutylamino)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methoxy-2-methylpropyl)amino)pyrimidin-4-yl)-1′,2′-dihydrospiro[cyclopropane-1,3′-pyrrolo[3,2-c]pyridin]-6′-yl)acetamide;
N-(1′-(6-(bicyclo[1.1.1]pentan-1-ylamino)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(((1-fluorocyclopropyl)methyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(((1-methoxycyclopropyl)methyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((oxetan-2-ylmethyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((oxetan-2-ylmethyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((3-fluoropropyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((3-methoxybutan-2-yl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(thietan-3-ylamino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(ethylamino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(((1s,3s)-3-fluorocyclobutyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-fluoropropyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((3-fluorobutyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methoxypropyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(((1s,3s)-3-methoxycyclobutyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methoxy-2-methylcyclopropyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(((1-methylazetidin-2-yl)methyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-(oxetan-3-yl)ethyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(spiro[2.2]pentan-1-ylamino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methoxycyclopropyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((1-methoxypropan-2-yl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((tetrahydrofuran-3-yl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-(dimethylamino)propyl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((tetrahydrothiophen-3-yl)amino)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((2-methylcyclopropyl)amino)pyrimidin-4-yl)-1′,2′-dihydrospiro[cyclopropane-1,3′-pyrrolo[3,2-c]pyridin]-6′-yl)acetamide;
N-(1′-(6-(3-(difluoromethyl)-4-methylpiperazin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3,3-difluoropyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-(2-methoxyethoxy)pyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-hydroxy-3-(trifluoromethyl)pyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3,4-dihydroxypyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-methoxypiperidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-hydroxypyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(dimethylamino)-4-methylpyridin-2-yl)-1′,2′-dihydrospiro[cyclopropane-1,3′ pyrrolo[3,2-c]pyridin]-6′-yl)acetamide;
N-(1′-(6-((2-methoxyethyl)amino)-4-methylpyridin-2-yl)-1′,2′-dihydrospiro[cyclopropane-1,3′-pyrrolo[3,2-c]pyridin]-6′-yl)acetamide;
N-(1′-(6-(3-(dimethylamino)pyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-hydroxyazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyanoazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-fluoroazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyano-3-(fluoromethyl)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyano-3-ethylazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-(difluoromethoxy)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyano-3-(2-methoxyethyl)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyano-3-methylazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-(difluoromethyl)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-cyano-3-(difluoromethyl)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(3-(methylsulfonyl)azetidin-1-yl)pyridin-2-yl)-1′,2′-dihydrospiro[cyclopropane-1,3′-pyrrolo[3,2-c]pyridin]-6′-yl)acetamide;
N-(1′-(6-(3-(ethylsulfonyl)azetidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(3-hydroxy-3-methylazetidin-1-yl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-morpholinopyridin-2-yl)-1′,2′-dihydrospiro[cyclopropane-1,3′-pyrrolo[3,2-c]pyridin]-6′-yl)acetamide;
N-(1′-(4-methyl-6-(4-methylpiperazin-1-yl)pyridin-2-yl)-1′,2′-dihydrospiro[cyclopropane-1,3′-pyrrolo[3,2-c]pyridin]-6′-yl)acetamide;
N-(1′-(4-methyl-6-(2-oxa-7-azaspiro[3.5]nonan-7-yl)pyridin-2-yl)-1′,2′-dihydrospiro[cyclopropane-1,3′-pyrrolo[3,2-c]pyridin]-6′-yl)acetamide;
N-(1′-(6-((3aR,6aS)-hexahydropyrrolo[3,4-c]pyrrol-2(1H)-yl)-4-methylpyridin-2-yl)-1′,2′-dihydrospiro[cyclopropane-1,3′-pyrrolo[3,2-c]pyridin]-6′-yl)acetamide;
N-(1′-(6-(hexahydropyrrolo[3,4-b]pyrrol-5(1H)-yl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(piperazin-1-yl)pyridin-2-yl)-1′,2′-dihydrospiro[cyclopropane-1,3′-pyrrolo[3,2-c]pyridin]-6′-yl)acetamide;
N-(1′-(6-(3-(2-hydroxyethyl)piperazin-1-yl)-4-methylpyridin-2-yl)-1′,2′-dihydrospiro[cyclopropane-1,3′-pyrrolo[3,2-c]pyridin]-6′-yl)acetamide;
N-(1′-(6-methyl-2-morpholinopyrimidin-4-yl)-1′,2′-dihydrospiro[cyclopropane-1,3′-pyrrolo[3,2-c]pyridin]-6′-yl)acetamide;
N-(1′-(2-(3-hydroxypyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-(dimethylamino)pyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyanoazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-fluoroazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyano-3-(fluoromethyl)azetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyano-3-ethylazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyano-3-(2-methoxyethyl)azetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyano-3-methylazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-(difluoromethyl)azetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-cyano-3-(difluoromethyl)azetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(3-(methylsulfonyl)azetidin-1-yl)pyrimidin-4-yl)-1′,2′-dihydrospiro[cyclopropane-1,3′-pyrrolo[3,2-c]pyridin]-6′-yl)acetamide;
N-(1′-(2-(3-(ethylsulfonyl)azetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-hydroxy-3-methylazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(4-methylpiperazin-1-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(2-oxa-7-azaspiro[3.5]nonan-7-yl)pyrimidin-4-yl)-1′,2′-dihydrospiro[cyclopropane-1,3′-pyrrolo[3,2-c]pyridin]-6′-yl)acetamide;
N-(1′-(2-(3,3-difluoropyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-methoxypiperidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(4-(cyclopropanecarbonyl)-4-methoxypiperidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(4-hydroxypiperidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(4-(2-methoxyethyl)piperazin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-methoxy-3-methylazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(2-methylazetidin-1-yl)pyrimidin-4-yl)-1′,2′-dihydrospiro[cyclopropane-1,3′-pyrrolo[3,2-c]pyridin]-6′-yl)acetamide;
N-(1′-(2-(3-hydroxy-3-methylpyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-hydroxyazetidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-methyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1,3-dimethyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1,5-dimethyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-methyl-1H-pyrazol-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(6-methylpyridin-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(6-methoxypyridin-3-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(1-methyl-1H-pyrazol-3-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(1-methyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1-cyclobutyl-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1-ethyl-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(5-(difluoromethyl)-1-methyl-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1-isopropyl-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(1-cyclopropyl-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(1-(oxetan-3-yl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(1H-pyrazol-3-yl)pyrimidin-4-yl)-1′,2′-dihydrospiro[cyclopropane-1,3′-pyrrolo[3,2-c]pyridin]-6′-yl)acetamide;
N-(1′-(2-(1-(cyclopropylmethyl)-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(2-(6-methoxypyridin-3-yl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(2-methylthiazol-5-yl)pyrimidin-4-yl)-1′,2′-dihydrospiro[cyclopropane-1,3′-pyrrolo[3,2-c]pyridin]-6′-yl)acetamide;
N-(1′-(4-methyl-6-(1-methyl-1H-pyrazol-3-yl)pyridin-2-yl)-1′,2′-dihydrospiro[cyclopropane-1,3′-pyrrolo[3,2-c]pyridin]-6′-yl)acetamide;
N-(1′-(4-methyl-6-(1-methyl-1H-pyrazol-4-yl)pyridin-2-yl)-1′,2′-dihydrospiro[cyclopropane-1,3′-pyrrolo[3,2-c]pyridin]-6′-yl)acetamide;
N-(1′-(4-methyl-6-(1-(methylsulfonyl)-1H-pyrazol-4-yl)pyridin-2-yl)-1′,2′-dihydrospiro[cyclopropane-1,3′-pyrrolo[3,2-c]pyridin]-6′-yl)acetamide;
N-(1′-(6-(1-(difluoromethyl)-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(6-(1-ethyl-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(1-methyl-3-(trifluoromethyl)-1H-pyrazol-4-yl)pyridin-2-yl)-1′,2′-dihydrospiro[cyclopropane-1,3′-pyrrolo[3,2-c]pyridin]-6′-yl)acetamide;
N-(1′-(6-(5-(difluoromethyl)-1-methyl-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(6-(1-isopropyl-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(6-(1-cyclopropyl-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(1-(oxetan-3-yl)-1H-pyrazol-4-yl)pyridin-2-yl)-1′,2′-dihydrospiro[cyclopropane-1,3′-pyrrolo[3,2-c]pyridin]-6′-yl)acetamide;
N-(1′-(6-(1-(cyclopropylmethyl)-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(6′-methoxy-4-methyl-[2,3′-bipyridin]-6-yl)-1′,2′-dihydrospiro[cyclopropane-1,3′-pyrrolo[3,2-c]pyridin]-6′-yl)acetamide;
N-(1′-(6-(1-(azetidin-3-yl)-1H-pyrazol-4-yl)-4-methylpyridin-2-yl)-1′,2′-dihydrospiro[cyclopropane-1,3′-pyrrolo[3,2-c]pyridin]-6′-yl)acetamide;
N-(1′-(2-(1-(azetidin-3-yl)-1H-pyrazol-4-yl)-6-methylpyrimidin-4-yl)-1′,2′-dihydrospiro[cyclopropane-1,3′-pyrrolo[3,2-c]pyridin]-6′-yl)acetamide;
N-(1′-(4-(1,1-difluoroethyl)pyrimidin-2-yl)-1′,2′-dihydrospiro[piperidine-4,3′-pyrrolo[3,2-c]pyridin]-6′-yl)acetamide;
N-(1′-(2-(1,1-difluoroethyl)pyrimidin-4-yl)-1′,2′-dihydrospiro[piperidine-4,3′-pyrrolo[3,2-c]pyridin]-6′-yl)acetamide;
N-(1′-(6-(1,1-difluoroethyl)pyridin-2-yl)-1′,2′-dihydrospiro[piperidine-4,3′-pyrrolo[3,2-c]pyridin]-6′-yl)acetamide;
N-(1′-(6-amino-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(4-(1,1-difluoroethyl)pyrimidin-2-yl)-1-methyl-1′,2′-dihydrospiro[piperidine-4,3′-pyrrolo[3,2-c]pyridin]-6′-yl)acetamide;
N-(1′-(2-(1,1-difluoroethyl)pyrimidin-4-yl)-1-methyl-1′,2′-dihydrospiro[piperidine-4,3′-pyrrolo[3,2-c]pyridin]-6′-yl)acetamide;
N-(1′-(6-(1,1-difluoroethyl)pyridin-2-yl)-1-methyl-1′,2′-dihydrospiro[piperidine-4,3′-pyrrolo[3,2-c]pyridin]-6′-yl)acetamide;
N-(1′-(4-methyl-6-1-methylhexahydropyrrolo[3,4-b]pyrrol-5(1H)-yl)pyridin-2-yl)-1′,2′-dihydrospiro[cyclopropane-1,3′-pyrrolo[3,2-c]pyridin]-6′-yl)acetamide;
3-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)-1,1-dimethylurea;
N-(1′-(6-(1-fluorocyclopropyl)-4-methylpyridin-2-yl)-1′,2′-dihydrospiro[cyclopropane-1,3′-pyrrolo[3,2-c]pyridin]-6′-yl)acetamide;
N-(1′-(6-(1,1-difluoroethyl)pyrimidin-4-yl)-1′,2′-dihydrospiro[cyclopropane-1,3′-pyrrolo[3,2-c]pyridin]-6′-yl)acetamide;
ethyl 2-(4-(6′-acetamidospiro[cyclopropane-1,3′-pyrrolo[3,2-c]pyridin]-1′(2′H)-yl)-6-methylpyrimidin-2-yl)-2,2-difluoroacetate;
N-(1′-(2-chloro-6-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-chloro-2-(2-fluoropropan-2-yl)pyrimidin-4-yl)-1′,2′-dihydrospiro[cyclopropane-1,3′-pyrrolo[3,2-c]pyridin]-6′-yl)acetamide;
N-(1′-(6-(cyclopropylamino)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(oxetan-3-ylamino)pyrimidin-4-yl)-1′,2′-dihydrospiro[cyclopropane-1,3′-pyrrolo[3,2-c]pyridin]-6′-yl)acetamide;
N-(1′-(2-(2-hydroxy-2-methylpropoxy)-6-methylpyrimidin-4-yl)-1′,2′-dihydrospiro[cyclopropane-1,3′-pyrrolo[3,2-c]pyridin]-6′-yl)acetamide;
N-(1′-(6-(1,1-difluoroethyl)-2-(2-methoxyethoxy)pyrimidin-4-yl)-1′,2′-dihydrospiro[cyclopropane-1,3′-pyrrolo[3,2-c]pyridin]-6′-yl)acetamide;
N-(1′-(2-(2-fluoroethoxy)-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoro-2-hydroxyethyl)-6-methylpyrimidin-4-yl)-1′,2′-dihydrospiro[cyclopropane-1,3′-pyrrolo[3,2-c]pyridin]-6′-yl)acetamide;
N-(1′-(6-(1,1-difluoro-2-hydroxyethyl)-4-methylpyridin-2-yl)-1′,2′-dihydrospiro[cyclopropane-1,3′-pyrrolo[3,2-c]pyridin]-6′-yl)acetamide;
ethyl 6-(6′-acetamidospiro[cyclopropane-1,3′-pyrrolo[3,2-c]pyridin]-1′(2′H)-yl)-2-(1,1-difluoroethyl)pyrimidine-4-carboxylate;
N-(1′-(2-(1,1-difluoroethyl)-6-(hydroxymethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-hydroxyethyl)pyrimidin-4-yl)-1′,2′-dihydrospiro[cyclopropane-1,3′-pyrrolo[3,2-c]pyridin]-6′-yl)acetamide;
6-(6′-acetamidospiro[cyclopropane-1,3′-pyrrolo[3,2-c]pyridin]-1′(2′H)-yl)-2-(1,1-difluoroethyl)pyrimidine-4-carboxylic acid;
N-(1′-(2-(1,1-difluoroethyl)-6-(3-hydroxypropyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(4,5-dihydrofuran-3-yl)pyrimidin-4-yl)-1′,2′-dihydrospiro[cyclopropane-1,3′-pyrrolo[3,2-c]pyridin]-6′-yl)acetamide;
(S)—N-(1′-(2-(1,1-difluoroethyl)-6-(tetrahydrofuran-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-((methylamino)methyl)pyrimidin-4-yl)-1′,2′-dihydrospiro[cyclopropane-1,3′-pyrrolo[3,2-c]pyridin]-6′-yl)acetamide;
N-(1′-(6-(azetidin-1-ylmethyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-methyl-1H-1,2,4-triazol-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-ethyl-5-fluoro-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(6-(1-cyanoethyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(6-(cyanomethyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoropropyl)-6-methylpyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-fluoropyrimidin-4-yl)-1′,2′-dihydrospiro[cyclopropane-1,3′-pyrrolo[3,2-c]pyridin]-6′-yl)acetamide;
N-(1′-(4-methyl-6-(1-methyl-2-oxabicyclo[2.1.1]hexan-4-yl)pyridin-2-yl)-1′,2′-dihydrospiro[cyclopropane-1,3′-pyrrolo[3,2-c]pyridin]-6′-yl)acetamide;
1-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)-3-methylurea;
1-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)urea;
1-(1′-(2-(1,1-difluoroethyl)-6-ethylpyrimidin-4-yl)-1′,2′-dihydrospiro[cyclopropane-1,3′-pyrrolo[3,2-c]pyridin]-6′-yl)urea;
N-(1′-(2-(1,1-difluoroethyl)-6-(5-fluoro-1-methyl-1H-pyrazol-4-yl)pyrimidin-4-yl)-1′,2′-dihydrospiro[cyclopropane-1,3′-pyrrolo[3,2-c]pyridin]-6′-yl)acetamide;
methyl (1′-(2-(1,1-difluoroethyl)-6-ethylpyrimidin-4-yl)-1′,2′-dihydrospiro[cyclopropane-1,3′-pyrrolo[3,2-c]pyridin]-6′-yl)carbamate;
N-(1′-(2-(2-fluoropropan-2-yl)-6-methylpyrimidin-4-yl)-1′,2′-dihydrospiro[cyclopropane-1,3′-pyrrolo[3,2-c]pyridin]-6′-yl)acetamide;
N-(1′-(6-methoxypyridin-2-yl)-1′,2′-dihydrospiro[cyclopropane-1,3′-pyrrolo[3,2-c]pyridin]-6′-yl)acetamide;
N-(1′-(6-ethyl-2-methylpyrimidin-4-yl)-1′,2′-dihydrospiro[cyclopropane-1,3′-pyrrolo[3,2-c]pyridin]-6′-yl)acetamide;
N-(1′-(6-(1,1-difluoro-2-methoxyethyl)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(1,1,2-trifluoroethyl)pyridin-2-yl)-1′,2′-dihydrospiro[cyclopropane-1,3′-pyrrolo[3,2-c]pyridin]-6′-yl)acetamide;
N-(1′-(2-(3-methoxycyclobutyl)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(1-methyl-2-oxabicyclo[2.1.1]hexan-4-yl)pyrimidin-4-yl)-1′,2′-dihydrospiro[cyclopropane-1,3′-pyrrolo[3,2-c]pyridin]-6′-yl)acetamide;
N-(1′-(6-methyl-2-(2-(methylsulfonyl)ethyl)pyrimidin-4-yl)-1′,2′-dihydrospiro[cyclopropane-1,3′-pyrrolo[3,2-c]pyridin]-6′-yl)acetamide;
N-(1′-(2-(2-oxabicyclo[2.1.1]hexan-4-yl)-6-methylpyrimidin-4-yl)-1′,2′-dihydrospiro[cyclopropane-1,3′-pyrrolo[3,2-c]pyridin]-6′-yl)acetamide;
(R)—N-(1′-(6-ethyl-2-(tetrahydrofuran-3-yl)pyrimidin-4-yl)-1′,2′-dihydrospiro[cyclopropane-1,3′-pyrrolo[3,2-c]pyridin]-6′-yl)acetamide;
N-(1′-(2-(2-oxabicyclo[2.1.1]hexan-4-yl)-6-ethylpyrimidin-4-yl)-1′,2′-dihydrospiro[cyclopropane-1,3′-pyrrolo[3,2-c]pyridin]-6′-yl)acetamide;
N-(1′-(2-(1,1-difluoro-2-methoxyethyl)pyrimidin-4-yl)-1′,2′-dihydrospiro[cyclopropane-1,3′-pyrrolo[3,2-c]pyridin]-6′-yl)acetamide;
(S)—N-(1′-(2-(1,1-difluoroethyl)-6-(3-hydroxy-3-methylpentyl)pyrimidin-4-yl)-1′,2′-dihydrospiro[cyclopropane-1,3′-pyrrolo[3,2-c]pyridin]-6′-yl)acetamide;
N-(1′-(6-(3-(tert-butoxy)propyl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(3-methoxypropyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(3-hydroxy-3-methylbutyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-pentylpyrimidin-4-yl)-1′,2′-dihydrospiro[cyclopropane-1,3′-pyrrolo[3,2-c]pyridin]-6′-yl)acetamide;
(Z)—N-(1′-(2-(1,1-difluoroethyl)-6-(2-ethoxyvinyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(2-ethoxyethyl)pyrimidin-4-yl)-1′,2′-dihydrospiro[cyclopropane-1,3′-pyrrolo[3,2-c]pyridin]-6′-yl)acetamide;
N-(1′-(4-fluoro-6-methylpyrimidin-2-yl)-1′,2′-dihydrospiro[cyclopropane-1,3′-pyrrolo[3,2-c]pyridin]-6′-yl)acetamide;
N-(1′-(2-fluoro-6-methylpyrimidin-4-yl)-1′,2′-dihydrospiro[cyclopropane-1,3′-pyrrolo[3,2-c]pyridin]-6′-yl)acetamide;
N-(1′-(4-methyl-6-(2,2,2-trifluoroethoxy)pyridin-2-yl)-1′,2′-dihydrospiro[cyclopropane-1,3′-pyrrolo[3,2-c]pyridin]-6′-yl)acetamide;
N-(1′-(6-isopropoxy-4-methylpyridin-2-yl)-1′,2′-dihydrospiro[cyclopropane-1,3′-pyrrolo[3,2-c]pyridin]-6′-yl)acetamide;
N-(1′-(6-ethoxy-4-methylpyridin-2-yl)-1′,2′-dihydrospiro[cyclopropane-1,3′-pyrrolo[3,2-c]pyridin]-6′-yl)acetamide;
N-(1′-(6-(2-methoxy-2-methylpropoxy)-4-methylpyridin-2-yl)-1′,2′-dihydrospiro[cyclopropane-1,3′-pyrrolo[3,2-c]pyridin]-6′-yl)acetamide;
N-(1′-(6-(((1R,3S)-3-hydroxycyclopentyl)oxy)-4-methylpyridin-2-yl)-1′,2′-dihydrospiro[cyclopropane-1,3′-pyrrolo[3,2-c]pyridin]-6′-yl)acetamide;
N-(1′-(6-(((1R,3S)-3-methoxycyclopentyl)oxy)-4-methylpyridin-2-yl)-1′,2′-dihydrospiro[cyclopropane-1,3′-pyrrolo[3,2-c]pyridin]-6′-yl)acetamide;
(R)—N-(1′-(4-methyl-6-((tetrahydrofuran-3-yl)oxy)pyridin-2-yl)-1′,2′-dihydrospiro[cyclopropane-1,3′-pyrrolo[3,2-c]pyridin]-6′-yl)acetamide;
N-(1′-(6-cyclobutoxy-4-methylpyridin-2-yl)-1′,2′-dihydrospiro[cyclopropane-1,3′-pyrrolo[3,2-c]pyridin]-6′-yl)acetamide;
N-(1′-(4-methyl-6-(oxetan-3-yloxy)pyridin-2-yl)-1′,2′-dihydrospiro[cyclopropane-1,3′-pyrrolo[3,2-c]pyridin]-6′-yl)acetamide;
N-(1′-(6-((1s,3s)-3-hydroxycyclobutoxy)-4-methylpyridin-2-yl)-1′,2′-dihydrospiro[cyclopropane-1,3′-pyrrolo[3,2-c]pyridin]-6′-yl)acetamide;
N-(1′-(6-((1s,3s)-3-methoxycyclobutoxy)-4-methylpyridin-2-yl)-1′,2′-dihydrospiro[cyclopropane-1,3′-pyrrolo[3,2-c]pyridin]-6′-yl)acetamide;
N-(1′-(6-((1s,3s)-3-cyanocyclobutoxy)-4-methylpyridin-2-yl)-1′,2′-dihydrospiro[cyclopropane-1,3′-pyrrolo[3,2-c]pyridin]-6′-yl)acetamide;
N-(1′-(6-((1s,3s)-3-(methoxymethyl)cyclobutoxy)-4-methylpyridin-2-yl)-1′,2′-dihydrospiro[cyclopropane-1,3′-pyrrolo[3,2-c]pyridin]-6′-yl)acetamide;
N-(1′-(6-((1r,3r)-3-fluorocyclobutoxy)-4-methylpyridin-2-yl)-1′,2′-dihydrospiro[cyclopropane-1,3′-pyrrolo[3,2-c]pyridin]-6′-yl)acetamide;
N-(1′-(4-methyl-6-((1s,3s)-3-methylcyclobutoxy)pyridin-2-yl)-1′,2′-dihydrospiro[cyclopropane-1,3′-pyrrolo[3,2-c]pyridin]-6′-yl)acetamide;
N-(1′-(6-((1s,3s)-3-fluorocyclobutoxy)-4-methylpyridin-2-yl)-1′,2′-dihydrospiro[cyclopropane-1,3′-pyrrolo[3,2-c]pyridin]-6′-yl)acetamide;
N-(1′-(4-methyl-6-(((1s,4s)-4-methyl-2-oxabicyclo[2.1.1]hexan-1-yl)methoxy)pyridin-2-yl)-1′,2′-dihydrospiro[cyclopropane-1,3′-pyrrolo[3,2-c]pyridin]-6′-yl)acetamide;
N-(1′-(6-(((1s,3s)-3-methoxycyclobutyl)methoxy)-4-methylpyridin-2-yl)-1′,2′-dihydrospiro[cyclopropane-1,3′-pyrrolo[3,2-c]pyridin]-6′-yl)acetamide;
N-(1′-(6-((1-methoxycyclobutyl)methoxy)-4-methylpyridin-2-yl)-1′,2′-dihydrospiro[cyclopropane-1,3′-pyrrolo[3,2-c]pyridin]-6′-yl)acetamide;
N-(1′-(6-(cyclopropylmethoxy)-4-methylpyridin-2-yl)-1′,2′-dihydrospiro[cyclopropane-1,3′-pyrrolo[3,2-c]pyridin]-6′-yl)acetamide;
N-(1′-(6-(((1R,3S)-3-fluorocyclopentyl)oxy)-4-methylpyridin-2-yl)-1′,2′-dihydrospiro[cyclopropane-1,3′-pyrrolo[3,2-c]pyridin]-6′-yl)acetamide;
N-(1′-(6-(cyclopentyloxy)-4-methylpyridin-2-yl)-1′,2′-dihydrospiro[cyclopropane-1,3′-pyrrolo[3,2-c]pyridin]-6′-yl)acetamide;
N-(1′-(6-(2-fluoroethoxy)-4-methylpyridin-2-yl)-1′,2′-dihydrospiro[cyclopropane-1,3′-pyrrolo[3,2-c]pyridin]-6′-yl)acetamide;
N-(1′-(6-(2,2-difluoropropoxy)-4-methylpyridin-2-yl)-1′,2′-dihydrospiro[cyclopropane-1,3′-pyrrolo[3,2-c]pyridin]-6′-yl)acetamide;
N-(1′-(6-(2-methoxyethoxy)-4-methylpyridin-2-yl)-1′,2′-dihydrospiro[cyclopropane-1,3′-pyrrolo[3,2-c]pyridin]-6′-yl)acetamide;
(R)—N-(1′-(4-methyl-6-(oxetan-2-ylmethoxy)pyridin-2-yl)-1′,2′-dihydrospiro[cyclopropane-1,3′-pyrrolo[3,2-c]pyridin]-6′-yl)acetamide;
(R)—N-(1′-(2-(1,1-difluoroethyl)-6-((1-methylpyrrolidin-3-yl)oxy)pyrimidin-4-yl)-1′,2′-dihydrospiro[cyclopropane-1,3′-pyrrolo[3,2-c]pyridin]-6′-yl)acetamide;
N-(1′-(2-(2-methoxyethoxy)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(2,2,2-trifluoroethoxy)pyrimidin-4-yl)-1′,2′-dihydrospiro[cyclopropane-1,3′-pyrrolo[3,2-c]pyridin]-6′-yl)acetamide;
N-(1′-(2-isopropoxy-6-methylpyrimidin-4-yl)-1′,2′-dihydrospiro[cyclopropane-1,3′-pyrrolo[3,2-c]pyridin]-6′-yl)acetamide;
N-(1′-(2-(3,3-difluorocyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(2-((2-oxaspiro[3.3]heptan-6-yl)oxy)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-((3-methyloxetan-3-yl)oxy)pyrimidin-4-yl)-1′,2′-dihydrospiro[cyclopropane-1,3′-pyrrolo[3,2-c]pyridin]-6′-yl)acetamide;
N-(1′-(6-methyl-2-(oxetan-3-ylmethoxy)pyrimidin-4-yl)-1′,2′-dihydrospiro[cyclopropane-1,3′-pyrrolo[3,2-c]pyridin]-6′-yl)acetamide;
N-(1′-(6-methyl-2-((1s,3s)-3-methylcyclobutoxy)pyrimidin-4-yl)-1′,2′-dihydrospiro[cyclopropane-1,3′-pyrrolo[3,2-c]pyridin]-6′-yl)acetamide;
N-(1′-(2-((1s,3s)-3-cyano-3-fluorocyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(2-(cyclopropylmethoxy)-6-methylpyrimidin-4-yl)-1′,2′-dihydrospiro[cyclopropane-1,3′-pyrrolo[3,2-c]pyridin]-6′-yl)acetamide;
N-(1′-(2-((1s,3s)-3-fluorocyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(6-methyl-2-(((1s,4s)-4-methyl-2-oxabicyclo[2.1.1]hexan-1-yl)methoxy)pyrimidin-4-yl)-1′,2′-dihydrospiro[cyclopropane-1,3′-pyrrolo[3,2-c]pyridin]-6′-yl)acetamide;
N-(1′-(2-(((1s,3s)-3-methoxycyclobutyl)methoxy)-6-methylpyrimidin-4-yl)-1′,2′-dihydrospiro[cyclopropane-1,3′-pyrrolo[3,2-c]pyridin]-6′-yl)acetamide;
N-(1′-(2-(2-methoxy-2-methylpropoxy)-6-methylpyrimidin-4-yl)-1′,2′-dihydrospiro[cyclopropane-1,3′-pyrrolo[3,2-c]pyridin]-6′-yl)acetamide;
(R)—N-(1′-(6-methyl-2-((tetrahydrofuran-3-yl)oxy)pyrimidin-4-yl)-1′,2′-dihydrospiro[cyclopropane-1,3′-pyrrolo[3,2-c]pyridin]-6′-yl)acetamide;
N-(1′-(2-((1s,3s)-3-cyanocyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(2-((1s,3s)-3-(methoxymethyl)cyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(2-((1r,3r)-3-fluorocyclobutoxy)-6-methylpyrimidin-4-yl)-1′,2′-dihydrospiro[cyclopropane-1,3′-pyrrolo[3,2-c]pyridin]-6′-yl)acetamide;
N-(1′-(2-((1-methoxycyclobutyl)methoxy)-6-methylpyrimidin-4-yl)-1′,2′-dihydrospiro[cyclopropane-1,3′-pyrrolo[3,2-c]pyridin]-6′-yl)acetamide;
N-(1′-(2-(cyclopentyloxy)-6-methylpyrimidin-4-yl)-1′,2′-dihydrospiro[cyclopropane-1,3′-pyrrolo[3,2-c]pyridin]-6′-yl)acetamide;
(R)—N-(1′-(6-methyl-2-(oxetan-2-ylmethoxy)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(3-fluoropyridin-4-yl)pyrimidin-4-yl)-1′,2′-dihydrospiro[cyclopropane-1,3′-pyrrolo[3,2-c]pyridin]-6′-yl)acetamide;
N-(1′-(6-(1-cyclobutyl-1H-pyrazol-4-yl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(difluoromethyl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-ethyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(5-(difluoromethyl)-1-methyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-isopropyl-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(6-(1-cyclopropyl-1H-pyrazol-4-yl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(oxetan-3-yl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(6-(1-(cyclopropylmethyl)-1H-pyrazol-4-yl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(2-morpholinoethyl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(2-(dimethylamino)ethyl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(2,2,2-trifluoroethyl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(6-(1-(cyanomethyl)-1H-pyrazol-4-yl)-2-(1,1-difluoroethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-(2-methoxyethyl)-1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1-fluoroethyl)pyrimidin-4-yl)-1′,2′-dihydrospiro[cyclopropane-1,3′-pyrrolo[3,2-c]pyridin]-6′-yl)acetamide;
N-(1′-(6-methyl-2-(oxetan-3-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(methoxymethyl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(2-fluoropropan-2-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(2-hydroxypropan-2-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoroethyl)-6-(1H-pyrazol-4-yl)pyrimidin-4-yl)-1′,2′-dihydrospiro[cyclopropane-1,3′-pyrrolo[3,2-c]pyridin]-6′-yl)acetamide;
N-(1′-(2-(1,1-difluoro-2-methoxyethyl)-6-methylpyrimidin-4-yl)-1′,2′-dihydrospiro[cyclopropane-1,3′-pyrrolo[3,2-c]pyridin]-6′-yl)acetamide;
(S)—N-(1′-(6-(3-cyanopyrrolidin-1-yl)-4-methylpyridin-2-yl)-1′,2′-dihydrospiro[cyclopropane-1,3′-pyrrolo[3,2-c]pyridin]-6′-yl)acetamide;
(R)—N-(1′-(6-(3-cyanopyrrolidin-1-yl)-4-methylpyridin-2-yl)-1′,2′-dihydrospiro[cyclopropane-1,3′-pyrrolo[3,2-c]pyridin]-6′-yl)acetamide;
(S)—N-(1′-(6-(3-cyano-3-methylpyrrolidin-1-yl)-4-methylpyridin-2-yl)-1′,2′-dihydrospiro[cyclopropane-1,3′-pyrrolo[3,2-c]pyridin]-6′-yl)acetamide;
(S)—N-(1′-(6-(3-hydroxy-3-methylpyrrolidin-1-yl)-4-methylpyridin-2-yl)-1′,2′-dihydrospiro[cyclopropane-1,3′-pyrrolo[3,2-c]pyridin]-6′-yl)acetamide;
N-(1′-(6-(4-(2-hydroxyethyl)piperazin-1-yl)-4-methylpyridin-2-yl)-1′,2′-dihydrospiro[cyclopropane-1,3′-pyrrolo[3,2-c]pyridin]-6′-yl)acetamide;
(R)—N-(1′-(2-(3-cyanopyrrolidin-1-yl)-6-methylpyrimidin-4-yl)-1′,2′-dihydrospiro[cyclopropane-1,3′-pyrrolo[3,2-c]pyridin]-6′-yl)acetamide;
(S)—N-(1′-(2-(3-cyanopyrrolidin-1-yl)-6-methylpyrimidin-4-yl)-1′,2′-dihydrospiro[cyclopropane-1,3′-pyrrolo[3,2-c]pyridin]-6′-yl)acetamide;
(R)—N-(1′-(2-(3-cyano-3-methylpyrrolidin-1-yl)-6-methylpyrimidin-4-yl)-1′,2′-dihydrospiro[cyclopropane-1,3′-pyrrolo[3,2-c]pyridin]-6′-yl)acetamide;
N-(1′-(2-(3-isobutylazetidin-1-yl)-6-methylpyrimidin-4-yl)-1′,2′-dihydrospiro[cyclopropane-1,3′-pyrrolo[3,2-c]pyridin]-6′-yl)acetamide; and
N-(1′-(6-methyl-2-((3aR,6aS)-5-methylhexahydropyrrolo[3,4-c]pyrrol-2(1H)-yl)pyrimidin-4-yl)-1′,2′-dihydrospiro[cyclopropane-1,3′-pyrrolo[3,2-c]pyridin]-6′-yl)acetamide;
   or a pharmaceutically acceptable salt thereof.

In a thirty-ninth embodiment, the present disclosure provides a pharmaceutical composition comprising a compound of the present disclosure (e.g., according to any one of the preceding embodiments), or a pharmaceutically acceptable salt thereof.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D2O, d6-acetone, d6-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or IPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have TYK2 activity. As used herein, “TYK2 inhibitory activity” refers to the ability of a compound or composition to induce a detectable decrease in TYK2 activity in vivo or in vitro (e.g., at least 10% decrease in TYK2 activity as measured by a given assay such as the bioassay described in the examples and known in the art).

In certain embodiments, the present disclosure provides a method of treating a disease or disorder responsive to inhibition of TYK2 activity (referred herein as “TYK2 mediated disease or disorder” or “disease or disorder mediated by TYK2”) in a subject in need of the treatment. The method comprises administering to the subject a compound described herein (e.g., a compound of Formula (I) according to any one of embodiments one to thirty-eight) or a pharmaceutically acceptable salt thereof or a pharmaceutical composition thereof.

In certain embodiments, the present disclosure provides the use of a compound described herein (e.g., a compound of Formula (I) according to any one of embodiments one to thirty-eight) or a pharmaceutically acceptable salt thereof, or a pharmaceutical composition comprising a compound described herein or a pharmaceutically acceptable salt thereof for the manufacture of a medicament for the treatment of a TYK2 mediated disorder or disease in a subject in need of the treatment.

In certain embodiments, the present disclosure provides a compound described herein (e.g., a compound of Formula (I) according to any one of embodiments one to thirty-eight) or a pharmaceutically acceptable salt thereof or a pharmaceutical composition comprising a compound described herein or a pharmaceutically acceptable salt thereof for use in the treatment of a TYK2 mediated disorder or disease in a subject in need of the treatment.

In some embodiments, the disease or disorder responsive to inhibition of TYK2 activity is inflammation, autoimmune disease, neuroinflammation, arthritis, rheumatoid arthritis, spondyloarthropathies, systemic lupus erythematosus, cutaneous lupus erythematosus, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, systemic sclerosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis or sunburn.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 μg and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄ alkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

According to a first process, compounds of Formula (I), may be prepared from compounds of Formulae (II′) and (III′), as shown in Scheme 1

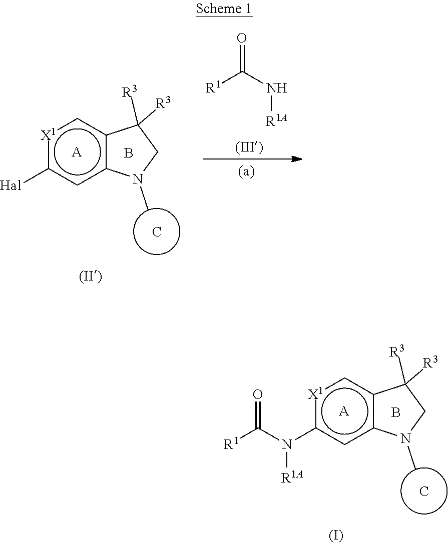

Hal is halogen, typically Cl or Br

The compound of Formula (I) may be prepared from the compounds of Formulae (II′) and (III′) according to process step (a) a Buchwald-Hartwig cross coupling reaction. Typical conditions comprise, reaction of the amide of Formula (III′) with the halide of Formula (II′) in the presence of a suitable inorganic base, a suitable palladium catalyst in the presence of suitable phosphine ligands, in a suitable solvent at elevated temperature, optionally under microwave irradiation. Preferred conditions comprise, reaction of the compounds of Formulae (II′) and (III′) in the presence of Brettphos Pd G3, Xantphos Pd G3, Xantphos Pd G4, RuPhos Pd G3, CPhos Pd G3, Josiphos, or Xantphos in combination with Pd₂(dba)₃, in the presence of a suitable base such as Cs₂CO₃, NaOtBu, Zn(OAc)₂, or K₃PO₄ in a suitable solvent such as dioxane, DMF or THF at between 70° C. and 120° C.

According to a second process, compounds of Formula (I), may be prepared from compounds of Formulae (IV′) and (V′), as shown in Scheme 2

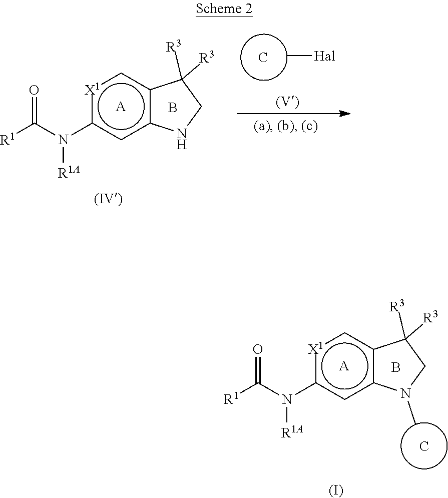

Hal is halogen, typically Cl or Br

The compound of Formula (I) may be prepared from the compound of Formula (IV′) and the halide of Formula (V′), according to process step (a), a Buchwald-Hartwig reaction, as previously described in Scheme 1.

Alternatively, the compound of Formula (I) may be prepared from the compound of Formula (IV′) and the halide of Formula (V′), by process step (b) an alkylation reaction, in the presence of a suitable inorganic or organic base and a suitable aprotic polar solvent at between rt and elevated temperature. Preferred conditions, comprise reaction of the compound of Formula (IV′) with the compound of Formula (V′) in the presence of NaH, KHMDS, KOtBu, K₃PO₄, Cs₂CO₃ or K₂CO₃ in DMSO, DMF or THF at between rt and 100° C.

Alternatively, the compound of Formula (I) may be prepared from the compound of Formula (IV′) and the halide of Formula (V′), by process step (c), an Ullmann-type, copper mediated coupling reaction. Typical conditions comprise, reaction of the compound of Formula (IV′) with the halide of Formula (V′), a copper catalyst, optionally in the presence of a suitable ligand, in the presence of a suitable inorganic or organic base in a suitable solvent at elevated temperature. Preferred conditions comprise, reaction of the compound of Formula (IV′) with the halide of Formula (V′) in the presence of Cu or CuI, optionally a suitable ligand such as N¹,N²-dimethylethane-1,2-diamine, or L-proline, a suitable inorganic base such as K₂CO₃ or K₃PO₄, optionally in a solvent such as dioxane or DMSO at between 40° C. and 120° C.

According to a third process, compounds of Formula (II′) and (IV′), may be prepared from compounds of Formulae (III′), (V′), (VI′), (VII′) and (VIII′) as shown in Scheme 3.

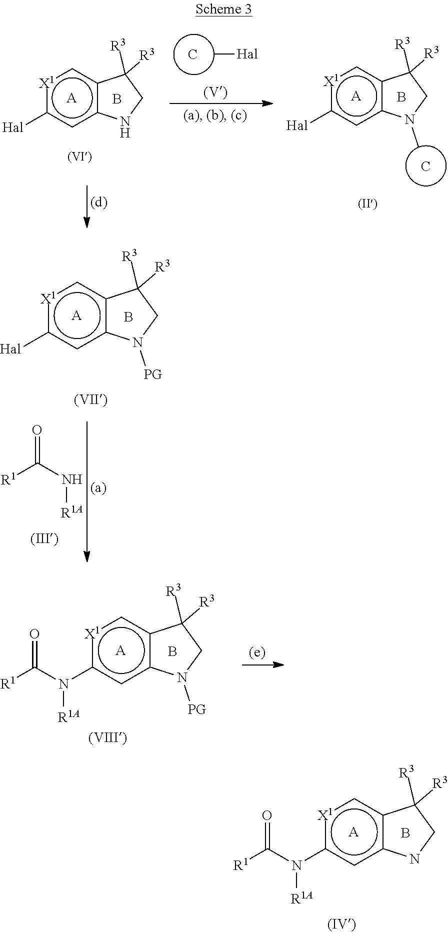

Hal is halogen, preferably Br or Cl

PG is a suitable N protecting group, typically a carbamate and preferably Boc.

The compound of Formula (II′) may be prepared from the compounds of Formulae (VI′) and (V′) according to process steps (a), (b) or (c) as previously described in Schemes 1 and 2.

The compound of Formula (VII′) may be prepared from the compound of Formula (VI′) according to process step (d) protection of a N atom. Typical conditions comprise reaction of the compound of Formula (VI′) with Boc₂O in the presence of a coupling agent, such as DMAP in a suitable solvent, such as THF at about rt.

The compound of Formula (VIII′) may be prepared from the compounds of Formula (VII′) and (III′) according to process step (a) as previously described in Scheme 1.

The compound of Formula (IV′) may be prepared from the compound of Formula (VIII′) according to process step (e) a deprotection reaction. Typical conditions comprise reaction of the compound of Formula (VIII′), with a suitable acid such as TFA or HCl in DCM at rt.

According to a fourth process, the compound of Formula (VI′), may be prepared from the compounds of Formulae (IX′), (X′), (XI′) and (XII′) as shown in Scheme 4.

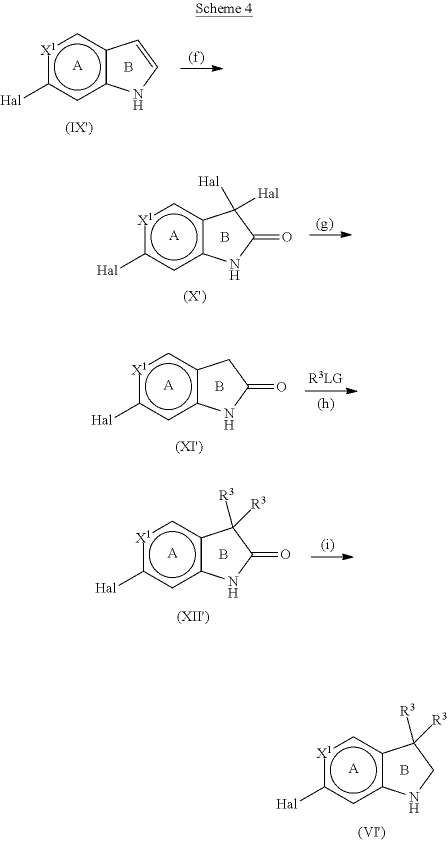

Hal is halogen, preferably Br or Cl

LG is a leaving group, typically a halide or sulfonate

The compound of Formula (X′) may be prepared from the compound of Formula (IX′) according to process step (f) an addition reaction. Preferred conditions comprise reaction of the compound of Formula (IX′) with a suitable halogenating agent, such as pyridinium tribromide in a suitable alcoholic solvent, such as t-BuOH at rt.

The compound of Formula (XI′) may be prepared from the compound of Formula (X′) according to process step (g) a reduction reaction. Preferred conditions comprise reaction of the compound of Formula (X′) in the presence of zinc and acetic acid at rt.

The compound of Formula (XII′) may be prepared from the compound of Formula (XI′) according to process step (h), an alkylation reaction. Typical conditions comprise reaction of the compound of Formula (XI′) with R³LG, in the presence of a strong organic or inorganic base, such as NaH, n-BuLi, LiHMDS or TMEDA in a suitable solvent, such as DMF or THF at rt.

The compound of Formula (VI′) may be prepared from the compound of Formula (XII′) according to process step (i), a carbonyl group reduction reaction. Preferred conditions comprise, reaction of the compound of Formula (XII′) with a reducing agent, typically LiAlH₄ or NaBH₄ in conjunction with BF₃·Et₂O in a suitable solvent such as THF at between rt and 60° C.

The compounds of Formulae (III′), (V′), (IX′) and R³LG, are either commercially available or may be prepared by analogy to methods known in the literature, or the methods described in the Experimental section below.

Compounds of Formulae (I), (II′), (IV′), (VI′) and (VIII′) may be converted to alternative compounds of Formulae (I), (II′), (IV′), (VI′) and (VIII′) by standard chemical transformations, known to those skilled in the art. Examples of these transformations include, but are not limited to:
reaction of an aryl or heteroaryl halide with an amine or alcohol (optionally in the presence of a suitable base) to provide an aryl or heteroaryl amine or ether,
reaction of an aryl or heteroaryl halide with an amine or alcohol according to process step (a), a Buchwald-Hartwig reaction, as previously described in Scheme 1 to provide an aryl or heteroaryl amine or ether,
reaction of an aryl or heteroaryl halide with an alkyl or arylboronate ester or acid, using Suzuki reaction conditions to provide an alkyl or aryl substituted aryl or heteroaryl group,
reaction of an aryl or heteroaryl halide with an alkyl or arylzinc reagent, using Negishi reaction conditions to provide an alkyl or aryl substituted aryl or heteroaryl group,
reaction of an aryl or heteroaryl halide with a substituted alkyne, using Sonogashira type reaction conditions to provide an alkenyl substituted aryl or heteroaryl group,
reaction of an aryl or heteroaryl halide with a cycloalkyl or 4 to 7 membered monocyclic heterocyclyl, substituted halide, or 1,3-dioxoisoindoline carboxylate in a transition metal photocatalysed reaction to provide a 4 to 7 membered monocyclic heterocyclyl substituted aryl or heteroaryl compound,
reaction of an aryl or heteroaryl halide with an alkylthiolate salt, to provide an alkylthio substituted aryl or heteroaryl group,
reduction of an alkene or alkyne, under hydrogenation conditions to provide the corresponding alkyl compound,
oxidation of an alkoxyvinyl group to provide an ester, using a suitable oxidising agent, such as KMnO₄,
cleavage of a vinyl ether under acidic conditions to provide a methyl ketone,
reaction of a ketone with a Grignard reagent to provide a tertiary alcohol,
oxidation of an alkylthio group, using MCPBA or trichlororuthenium hydrate with IO₄ to provide an alkylsulfonyl group,
alkylation of an aryl or heteroaryl alcohol, according to process step (b) an alkylation reaction as previously described in Scheme 2,
Alkylation of a heteroatom, such as 0 or N using an alkyl halide in the presence of an inorganic base to provide a secondary amine or ether,
C—H alkylation of a heteroaryl group with a 4 to 7 membered monocyclic or heterocyclic halide, using redox catalysis, to provide a 4 to 7 membered monocyclic or heterocyclic heteroaryl group,
reductive amination of a N atom to provide a secondary amine,
fluorination of an alcohol or carbonyl group using a fluorinating agent, such as DAST,
fluorination of an aryl or heteroaryl chloride using TBAF to provide an aryl or heteroaryl fluoride,
chlorination of a primary alcohol with a chlorinating agent, such as SOCl₂ to provide an alkyl chloride,
hydrolysis of a carboxylic ester to provide a carboxylic acid, or
dehalogenation of a heteroaryl halide, using a palladium catalyst, to provide a heteroaryl.

It will be appreciated by those skilled in the art that it may be necessary to utilise a suitable protecting group strategy for the preparation of compounds of Formula (I). Typical protecting groups may comprise, 2,4-dimethoxybenzyl or a carbamate group, preferably Boc for the protection of amines, or a benzyl group for the protection of a phenolic OH.

It will be further appreciated that it may be necessary or desirable to carry out the transformations in a different order from that described in the schemes, or to modify one or more of the transformations, to provide the desired compound of the invention.

### EXEMPLIFICATION

### Abbreviations

AcOH=acetic acid;
AIBN=2,2′-azobis(2-methylpropionitrile);
Aq.=aqueous;
BF₃·Et₂O=boron trifluoride diethyl etherate;
Bn=benzyl;
BnOH=benzyl alcohol;
Boc=tert-butoxy carbonyl;
Boc₂O=di-tert-butyl dicarbonate;
br=broad;
Brettphos Pd G3=[(2-di-cyclohexylphosphino-3,6-dimethoxy-2′,4′,6′-triisopropyl-1,1′-biphenyl)-2-(2′-amino-1,1′-biphenyl)]palladium(II) methanesulfonate;
t-BuOH=tert-butanol;
t-BuONa=sodium tert-butoxide;
n-BuLi=n-butyl lithium;
° C.=degrees Celsius;
CDCl₃=deutero-chloroform;
CS₂=carbon disulfide;
Cs₂CO₃=cesium carbonate;
CuI=copper (I) iodide;
δ=chemical shift;
d=doublet;
DAST=(diethylamino)sulfur trifluoride;
dd=double doublet;
DCM=dichloromethane;
DIPEA=N-ethyldiisopropylamine or N,N-diisopropylethylamine;
DMAP=4-(Dimethylamino)pyridine;
DME=1,2-dimethoxyethane;
DMF=N,N-dimethylformamide;
DMSO=Dimethylsulfoxide;
DMSO-d₆=hexadeuterodimethyl sulfoxide;
dtbbpy=4-tert-butyl-2-(4-tert-butylpyridin-2-yl)pyridine;
Et=ethyl;
EtOH=ethanol;
EtOAc=ethyl acetate;
Eq.=equivalent;
FeSO₄=iron (II) sulfate;
g=gram;
HCl=hydrochloric acid;
HCO₂H=formic acid;
¹H NMR=proton nuclear magnetic resonance;
H₂O=water;
HPLC=high pressure liquid chromatography;
h=hour;
IPA=2-propanol;
K₂CO₃=potassium carbonate;
KF=potassium fluoride;
KHMDS=potassium hexamethyldisilazide;
KOH=potassium hydroxide;
KOtBu=potassium tert-butoxide;
K₃PO₄=potassium phosphate tribasic;
L=litre;
LCMS=liquid chromatography mass spectrometry;
LiAlH₄=lithium aluminium hydride;
LiHMDS=lithium hexamethyldisilylazide;
m=multiplet;
M=molar;
MCPBA=3-chloroperoxybenzoic acid;
Me=methyl;
MeCN=acetonitrile;
MeI=iodomethane;
MeOH=methanol;
MeOH-d₄=deutero-methanol;
mg=milligram;
MgSO₄=magnesium sulfate;
MHz=mega Hertz;
mins=minutes;
mL=millilitres;
mmol=millimole;
MS m/z=mass spectrum peak;
N₂=nitrogen;
NaBH₄=sodium borohydride;
NaBH₃CN=sodium cycanoborohydride;
Na₂CO₃=sodium carbonate;
NaH=sodium hydride;
NaHCO₃=sodium bicarbonate;
NaOH=sodium hydroxide;
NaOtBu=sodium tert-butoxide;
NaOMe=sodium methoxide;
Na₂SO₃=sodium thiosulfate;
Na₂SO₄=sodium sulfate;
NBS=N-bromosuccinimide;
NH₃=ammonia;
NH₄Cl=ammonium chloride;
NH₄OH is ammonium hydroxide;
NiCl₂·glyme=dichloro(dimethoxyethane)nickel;
PE=petroleum ether;
Pd(dppf)Cl₂=[1,1′-bis(diphenylphosphino)ferrocene]dichloropalladium(II);
Pd(DPEPhos)Cl₂=dichloro[bis(2-(diphenylphosphino)phenyl)ether]palladium(II);
Pd(OAc)₂=palladium (II) acetate;
Pd₂(dba)₃=tris(dibenzylideneacetone)dipalladium (0);
POCl₃=phosphorus (V) oxychloride;
q=quartet;
rt=room temperature;
RT=retention time;
RuPhos Pd G3=(2-dicyclohexylphosphino-2′,6′-diisopropoxy-1,1′-biphenyl)[2-(2′-amino-1,1′-biphenyl)]palladium(II) methanesulfonate;
s=singlet;
sat.=saturated;
soln.=solution;
t=triplet;
TEA=triethylamine;
TFA=trifluoroacetic acid;
THF=tetrahydrofuran;
TLC=thin layer chromatography;
TMEDA=1,2-bis(dimethylamino)ethane;
μL=micro litres;
μmol=micromole;
Xantphos=4,5-Bis(diphenylphosphino)-9,9-dimethylxanthene;
Xantphos Pd G3=[(4,5-bis(diphenylphosphino)-9,9-dimethylxanthene)-2-(2′-amino-1,1′-biphenyl)]palladium(II) methanesulfonate;
Zn(OAc)₂=Zn acetate.

### General Methods

### Preparation 1

### 3,3-dibromo-6-chloro-1,3-dihydro-2H-pyrrolo[3,2-c]pyridin-2-one

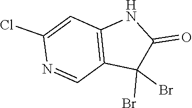 [SMILES: O=C1Nc2cc(Cl)ncc2C1(Br)Br]

To a solution of 6-chloro-1H-pyrrolo[3,2-c]pyridine (8.0 g, 52.4 mmol) in t-BuOH (250 mL) was added pyridinium tribromide (50.4 g, 157 mmol) over 15 mins. The reaction mixture was stirred at 25° C. for 16 h. The mixture was diluted with water (400 mL) and extracted with EtOAc (100 mL×3). The combined organic layers were washed with brine (200 mL), dried over Na₂SO₄, filtered and concentrated under vacuum to give 3,3-dibromo-6-chloro-1,3 -dihydro-2H-pyrrolo[3,2-c]pyridin-2-one (16.7 g, 97.6% yield) as a yellow solid. ¹H NMR: (400 MHz, CDCl₃) δ: ppm 9.07 (s, 1H), 8.56 (s, 1H), 7.01 (s, 1H).

### Alternative Synthesis

To a stirred solution of t-BuOH and THF (5.0 L) was added 6-chloro-1H-pyrrolo[3,2-c]pyridine (1.70 kg, 11.14 mol) and H₂O (1.7 L) at rt. After stirring for 20 min, NBS (6.346 kg, 35.65 mol) was added in batches over 4 h at 20-25° C. and the on complete addition, the reaction was stirred for a further 2 h. The reaction mixture was poured into ice water (25 L) and the resulting solid was filtered off and dried at 45-50° C. for 24 h to give 3,3-dibromo-6-chloro-1,3-dihydro-2H-pyrrolo[3,2-c]pyridin-2-one (3.40 kg, 93.5%) as a yellow solid. HNMR (400 MHz, DMSO-d₆) δ (ppm): 12.01 (brs, 1H), 8.58 (s, 1H), 7.05 (d, 1H)

### Preparation 2

### 6-chloro-1,3-dihydro-2H-pyrrolo[3,2-c]pyridin-2-one

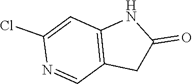 [SMILES: O=C1Cc2cnc(Cl)cc2N1]

To a solution of 3,3-dibromo-6-chloro-1,3-dihydro-2H-pyrrolo[3,2-c]pyridin-2-one (Preparation 1, 16.5 g, 50.6 mmol) in AcOH (150 mL) was added zinc (33.1 g, 506 mmol) and the reaction mixture was stirred at rt for 50 min. The mixture was filtered and concentrated under vacuum, diluted with sat. NaHCO₃ (150 mL) and extracted with EtOAc (200 mL×5). The combined organic layers were washed with H₂O (200 mL×3) and brine (100 mL×2). The organic layers were dried over Na₂SO₄, filtered and concentrated under vacuum to give 6-chloro-1,3-dihydro-2H-pyrrolo[3,2-c]pyridin-2-one (7.0 g, 82.1% yield) as a yellow solid. ¹H NMR: (400 MHz, DMSO-d₆) δ: ppm 10.95 (s, 1H), 8.03 (s, 1H), 6.82 (s, 1H), 3.52 (s, 2H).

### Alternative Synthesis

To a stirred solution of 3,3-dibromo-6-chloro-1,3-dihydro-2H-pyrrolo[3,2-c]pyridin-2-one (Preparation 1, 3.40 kg, 0.418 mol) in H₂O (20 L) was added a solution of Na₂SO₃ (5.252 kg, 41.67 mol) in H₂O (15 L) over 1.5 h at rt. The mixture was stirred at 25° C. for 2 h, then filtered. The resulting solid was washed with H₂O and dried at 45-50° C. for 24 h to give 6-chloro-1,3-dihydro-2H-pyrrolo[3,2-c]pyridin-2-one (1.70 kg, 96.8%) as an off-white solid. 1HNMR (400 MHz, DMSO-d₆) δ (ppm): 10.99 (brs, 1H), 8.06 (s, 1H), 6.86 (s, 1H), 3.56 (s, 2H)

### Preparation 3

### 6′-chlorospiro[cyclopropane-1,3′-pyrrolo[3,2-c]pyridin]-2′(1′H)-one

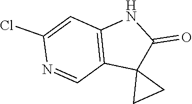 [SMILES: O=C1Nc2cc(Cl)ncc2C12CC2]

A solution of 6-chloro-1,3-dihydro-2H-pyrrolo[3,2-c]pyridin-2-one (Preparation 2, 2.0 g, 11.9 mmol) in DMF (10 mL) was added dropwise to a stirred suspension NaH (2.9 g, 71.2 mmol, 60% purity) in DMF (30 mL) under N₂ at 0° C. The reaction mixture was stirred for 15 min before dropwise addition of a solution of 1,2-dibromoethane (6.7 g, 35.6 mmol) in dry DMF (4.0 mL). The brown reaction mixture was stirred at rt for 18 h, then filtered and concentrated under vacuum before addition of H₂O (400 mL) and extraction with EtOAc (200 mL×5). The combined organic layers were washed with H₂O (200 mL×3) and brine (100 mL×2), dried over Na₂SO₄, filtered and concentrated under reduced pressure. The crude product was slurred in EtOAc and PE, filtered and dried to give 6′-chlorospiro[cyclopropane-1,3′-pyrrolo[3,2-c]pyridin]-2′(1′H)-one (1 g, 43.3% yield) as a yellow solid. ¹H NMR: (500 MHz, DMSO-d₆) δ: ppm 11.19 (s, 1H), 7.94 (s, 1H), 6.96 (s, 1H), 1.78-1.75 (m, 2H), 1.57-1.54 (m, 2H).

### Alternative Synthesis

A mixture of THF (7.0 L), 6-chloro-1,3-dihydro-2H-pyrrolo[3,2-c]pyridin-2-one (Preparation 2, 650 g, 3.856 mol) and diisopropylamine (819.33 g, 8.097 mol) was cooled to −30° C. n-BuLi (6.169 L, 15.42 mol, 2.5 M in n-Hexane) was added dropwise under N₂ atmosphere. After stirring at this temperature for 4 h, the mixture was warmed to 10° C. 1,2-Dibromoethane (1.449 kg, 7.711 mol) was added dropwise and the resulting mixture was stirred at 30° C.-35° C. for 48 h. The reaction mixture was quenched with 4M aq. HCl (30 L) and the pH was adjusted to 8-9 with saturated aq. Na₂CO₃. The mixture was filtered and the resulting solid was dried to give 6′-chlorospiro[cyclopropane-1,3′-pyrrolo[3,2-c]pyridin]-2′(1′H)-one (650 g, 86.6%) as a yellow solid. ¹HNMR (400 MHz, DMSO-d₆) δ (ppm): 11.20 (brs, 1H), 7.93 (s, 1H), 6.96 (s, 1H), 1.77-1.74 (m, 2H), 1.57-1.54 (m, 2H)

### Preparation 4

### 6′-chloro-1′,2′-dihydrospiro[cyclopropane-1,3′-pyrrolo[3,2-c]pyridine]

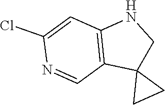 [SMILES: Clc1cc2c(cn1)C1(CC1)CN2]

A solution of 6′-chlorospiro[cyclopropane-1,3′-pyrrolo[3,2-c]pyridin]-2′(1′H)-one (Preparation 3, 850 mg, 4.37 mmol) and LiAlH₄ (547 mg, 14.4 mmol) in THF (40 mL) was stirred at 60° C. for 10 min. The mixture was diluted with water (70 mL) and extracted with EtOAc (60 mL×3). The combined organic layers were washed with brine (40 mL), dried over Na₂SO₄, filtered and concentrated under vacuum to give the crude product which was purified by chromatography on silica gel (PE/EtOAc 1/0 to 2/1) to give 6′-chloro-1′,2′-dihydrospiro[cyclopropane-1,3′-pyrrolo[3,2-c]pyridine](470 mg, 59.6% yield) as a yellow solid. ¹H NMR: (500 MHz, DMSO-d₆) δ: ppm 7.32 (s, 1H), 6.91 (s, 1H), 6.31 (s, 1H), 3.60 (s, 2H), 1.02-1.01 (m, 2H), 1.01-0.98 (m, 2H).

### Alternative Synthesis

To a stirred solution of 6′-chlorospiro[cyclopropane-1,3′-pyrrolo[3,2-c]pyridin]-2′(1′H)-one (Preparation 3, 1.2 kg, 6.166 mol) in THF (8.0 L) was added NaBH₄ (466.55 g, 12.33 mol) in batches at 25° C. over a period of 20 min. BF₃·Et₂O (2.45 kg, 17.265 mol) was then added dropwise at 20° C. under N₂ atmosphere over 5 h and after complete addition, the reaction was stirred for a further 2 h. The reaction mixture was quenched with 6N aq. HCl (20 L) and stirred vigorously at 25° C. for 16 h. The mixture was basified to pH=9 with 6 N aq.NaOH with ice cooling, then filtered. The filter cake was dissolved in 2 N aq.HCl (7 L), the solution was stirred at 25° C. for 2 h, then basified to pH=9 with 6 N aq.NaOH with ice cooling. The resulting solid was filtered off and dried to give 6′-chloro-1′,2′-dihydrospiro[cyclopropane-1,3′-pyrrolo[3,2-c]pyridine](974.50 g, 87.50%) as a yellow solid. ¹HNMR (400 MHz, DMSO-d₆) δ (ppm): 7.32 (s, 1H), 6.92 (brs, 1H), 6.30 (s, 1H), 3.59 (s, 2H), 1.01-0.98 (m, 4H)

### Preparation 5

### tert-butyl 6′-chlorospiro[cyclopropane-1,3′-pyrrolo[3,2-c]pyridine]-1′(2′H)-carboxylate

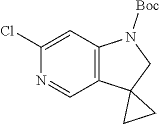 [SMILES: CC(C)(C)OC(=O)N1CC2(CC2)c2cnc(Cl)cc21]

To a solution of 6′-chloro-1′,2′-dihydrospiro[cyclopropane-1,3′-pyrrolo[3,2-c]pyridine] (Preparation 4, 300 mg, 1.66 mmol) and DMAP (20.3 mg, 0.166 mmol) in THF (2 mL) was added Boc₂0 (725 mg, 3.32 mmol) and TEA (420 mg, 4.15 mmol). The reaction mixture was stirred at 25° C. for 1 h. The mixture was concentrated and purified by chromatography on silica gel (PE/EtOAc 15/1 to 3/1) to give tert-butyl 6′-chlorospiro[cyclopropane-1,3′-pyrrolo[3,2-c]pyridine]-1′(2′H)-carboxylate (274 mg, 58.8% yield) as a white solid. ¹H NMR: (400 MHz, CDCl₃) δ: ppm 7.84-7.71 (m, 1H), 7.59 (s, 1H), 3.97 (s, 2H), 1.63 (s, 9H), 1.18-1.10 (m, 4H).

### Alternative Synthesis

6′-Chloro-1′,2′-dihydrospiro[cyclopropane-1,3′-pyrrolo[3,2-c]pyridine] (Preparation 4, 800 g, 4.43 mol) was dissolved in THF (5 L). DMAP (541.6 g, 4.43 mol) and Boc₂0 (2.416 kg, 11.072 mol) were added and the mixture was stirred at 20° C. for 5 h. EtOAc (4 L) was added and the mixture was washed with saturated aq. NH₄Cl (2 L×5). The organic phase was dried over anhydrous MgSO₄, filtered and concentrated under vacuum. The crude product was triturated with n-Heptane (1 L) at ambient temperature for 0.5 h, then filtered to give tert-butyl 6′-chlorospiro[cyclopropane-1,3′-pyrrolo[3,2-c]pyridine]-1′(2′H)-carboxylate (900 g, 72.38%) as an off-white sold. ¹HNMR (400 MHz, DMSO-d₆) δ (ppm): 7.73 (s, 1H), 7.47 (s, 1H), 3.98 (s, 2H), 1.51 (s, 9H), 1.17-1.13 (m, 4H)

### Preparation 6

### tert-butyl 6′-acetamidospiro[cyclopropane-1,3′-pyrrolo[3,2-c]pyridine]-1′(2′H)-carboxylate

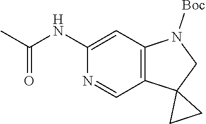 [SMILES: CC(=O)Nc1cc2c(cn1)C1(CC1)CN2C(=O)OC(C)(C)C]

To a solution of tert-butyl 6′-chlorospiro[cyclopropane-1,3′-pyrrolo[3,2-c]pyridine]-1′(2′H)-carboxylate (Preparation 5, 274 mg, 0.976 mmol) in dioxane (3 mL) was added acetamide (86.5 mg, 1.46 mmol), Cs₂CO₃ (954 mg, 2.93 mmol), Xantphos (113 mg, 0.195 mmol) and Pd₂(dba)₃ (89.4 mg, 0.0976 mmol). The reaction mixture was stirred at 100° C. for 16 h under N₂. The mixture was treated with H₂O (10 mL) and extracted with EtOAc (15 mL×3). The organic layers were washed with brine (20 mL), dried over Na₂SO₄, filtered and concentrated under reduced pressure. The crude product was purified by chromatography on silica gel (PE/EtOAc 15/1 to 0/1) to give tert-butyl 6′-acetamidospiro[cyclopropane-1,3′-pyrrolo[3,2-c]pyridine]-1′(2′H)-carboxylate (269 mg, 90.7% yield) as a yellow solid. LCMS m/z=304.1 [M+H]⁺. ¹H NMR: (400 MHz, CDCl₃) δ: ppm 8.49-8.35 (m, 2H), 7.39 (s, 1H), 3.97 (s, 2H), 2.15 (s, 3H), 1.62 (s, 9H), 1.09-1.04 (m, 4H).

### Alternative Synthesis

Into a solution of tert-butyl 6′-chlorospiro[cyclopropane-1,3′-pyrrolo[3,2-c]pyridine]-1′(2′H)-carboxylate (Preparation 5, 150 g, 534.285 mmol) in dioxane (1.20 L) was added acetamide (63.12 g, 1.069 mol), K₂CO₃ (134.7 g, 1.069 mol), Pd₂(dba)₃ (34.25 g, 37.4 mmol) and XantPhos (43.28 g, 74.8 mmol). The reaction mixture was degassed with N₂ and stirred at 100° C. for 16 h. The reaction mixture was diluted with EtOAc (10 L) and filtered through a pad of Celite®. The filtrate was washed with water (3 L×3), the organic phase was dried over anhydrous MgSO₄, filtered and concentrated in vacuo. The crude product was triturated with PE/EtOAc=8:1, filtered and air dried to give tert-butyl 6′-acetamidospiro[cyclopropane-1,3′-pyrrolo[3,2-c]pyridine]-1′(2′H)-carboxylate as a yellow solid. ¹HNMR (400 MHz, DMSO-d₆) δ (ppm): 10.30 (brs, 1H), 8.34 (s, 1H), 7.62 (d, 1H), 3.93 (s, 2H), 2.05 (s, 3H), 1.53 (s, 9H), 1.10-1.06 (m, 4H)

### Preparation 7

### N-(1′,2′-dihydrospiro[cyclopropane-1,3′-pyrrolo[3,2-c]pyridin]-6′-yl)acetamide

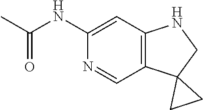

To a solution of tert-butyl 6′-acetamidospiro[cyclopropane-1,3′-pyrrolo[3,2-c]pyridine]-1′(2′H)-carboxylate (Preparation 6, 248 mg, 0.818 mmol) in DCM (25 mL) was added TFA (5 mL). The mixture was stirred at 25° C. for 2 h. The solution was adjusted pH to 7-8 with NH₄₀H. The mixture was concentrated and purified by prep-HPLC (Column: Boston Green ODS 150*30 mm*5 um; Condition: water (NH₄HCO₃)-MeCN; Begin B: 15; End B: 45; Gradient Time (min): 11; Flow Rate (ml/min): 25) to give N-(1′,2′-dihydrospiro[cyclopropane-1,3′-pyrrolo[3,2-c]pyridin]-6′-yl)acetamide (60 mg, 36.1% yield) as a white solid. LCMS m/z=204.1 [M+H]⁺. ¹H NMR: (500 MHz, CDCl₃) δ: ppm 7.98 (s, 1H), 7.37 (s, 1H), 7.03 (s, 1H), 4.32 (s, 1H), 3.72-3.62 (m, 2H), 2.10 (s, 3H), 1.03-0.94 (m, 4H).

### Alternative Synthesis

To a solution of tert-butyl 6′-acetamidospiro[cyclopropane-1,3′-pyrrolo[3,2-c]pyridine]-1′(2′H)-carboxylate (Preparation 6, 800 g, 2.637 mol) in DCM (3.0 L) was added TFA (3.007 kg, 26.372 mol) at rt and the reaction was stirred at 25° C. for 15 h. The mixture was concentrated and the residue was diluted with H₂O (3.0 L). The solution was basified to pH=9 with saturated aq.Na₂CO₃. After filtration, the filter cake was washed with saturated aq.Na₂CO₃ (2.0 L). After drying at 45-50° C. for 24 h, the solid was slurried with DCM/n-Heptane (1.0 L/1.0 L) at rt. The solid was collected by filtration and dried to give N-(1′,2′-dihydrospiro[cyclopropane-1,3′-pyrrolo[3,2-c]pyridin]-6′-yl)acetamide (510.4 g, 95.2%) as a yellow solid. ¹HNMR (400 MHz, DMSO-d₆) δ (ppm): 10.01 (brs, 1H), 7.28 (s, 1H), 7.20 (s, 1H), 6.64 (s, 1H), 3.54 (s, 2H), 2.01 (s, 3H), 0.96-0.92 (m, 4H)

### Preparation 8

### 6′-chloro-2-methylspiro[cyclopropane-1,3′-pyrrolo[3,2-c]pyridin]-2′(1′H)-one

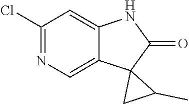

To a solution of 6-chloro-1,3-dihydro-2H-pyrrolo[3,2-c]pyridin-2-one (Preparation 2, 500 mg, 2.97 mmol) in THF (20 mL) was added butyllithium (2.5 M, 2.97 mL) at −78° C. The reaction mixture was stirred at −78° C. for 1 h followed by addition of 4-methyl-1,3,2-dioxathiolane 2,2-dioxide (451 mg, 3.26 mmol) whilst the temperature was maintained below −50° C. The reaction was warmed to rt overnight, then quenched with water and partitioned between EtOAc and water. The aqueous solution was extracted with EtOAc and the combined organic layers were washed with brine, dried over Na₂SO₄, filtered, and concentrated under reduced pressure. The crude was purified by chromatography on silica gel (0-80% EtOAc-EtOH 3:1 with 2% NH₄OH in heptane) to give 6′-chloro-2-methylspiro[cyclopropane-1,3′-pyrrolo[3,2-c]pyridin]-2′(1′H)-one (115 mg, 19% yield). LCMS m/z=209.1 [M+H]⁺.

### Preparation 9

### 6′-chlorospiro[cyclobutane-1,3′-pyrrolo[3,2-c]pyridin]-2′(1′H)-one

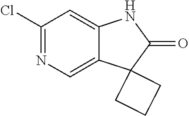 [SMILES: O=C1Nc2cc(Cl)ncc2C12CCC2]

To a solution of 6-chloro-1,3-dihydro-2H-pyrrolo[3,2-c]pyridin-2-one (Preparation 2, 1.0 g, 5.93 mmol) and TMEDA (1.72 g, 14.8 mmol) in THF (40 mL) was added butyllithium (2.5 M, 5.93 mL) dropwise at −78° C. The mixture was stirred at −78° C. for 10 min followed by stirring at rt for 30 min. 1,3-Diiodopropane (4.4 g, 14.8 mmol) was added dropwise at −50° C. to −30° C. The reaction was allowed to warm up to rt overnight, then filtered. The filtrate was concentrated under reduced pressure and diluted with EtOAc. The precipitate was filtered and rinsed with EtOAc to give a solid (304 mg). The filtrate was concentrated under reduced pressure, then purified by chromatography on silica gel (0-80% EtOAc-EtOH 3:1 with 2% NH₄OH in heptane) to give 242 mg of 6-chlorospiro[1H-pyrrolo[3,2-c]pyridine-3,1′-cyclobutane]-2-one (in total 546 mg, 44% yield) as a white powder. LCMS m/z=209.1 [M+H]⁺.

### Preparation 10

### 6′-chlorospiro[cyclopentane-1,3′-pyrrolo[3,2-c]pyridin]-2′(1′H)-one

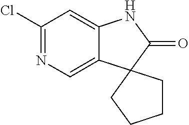 [SMILES: O=C1Nc2cc(Cl)ncc2C12CCCC2]

To a solution of LiHMDS (1.0 M, 14.8 mL) in THF (10 mL) was added 6-chloro-1,3-dihydro-2H-pyrrolo[3,2-c]pyridin-2-one (Preparation 2, 1.0 g, 5.93 mmol) at −78° C. The reaction mixture was stirred at −78° C. for 5 min, then warmed up to −50° C. and stirred for 30 min. 1,4-Dichlorobutane (829 mg, 6.5 mmol) was dropwise added at 0° C. The reaction mixture was allowed to warm up to rt and heated to reflux overnight. The mixture was concentrated under reduced pressure and partitioned between ether and sat. aq. NH₄Cl. The ether layer was concentrated under reduced pressure and purified by chromatography on silica gel (0-100% EtOAc in heptane) to give 6′-chlorospiro[cyclopentane-1,3′-pyrrolo[3,2-c]pyridin]-2′(1′H)-one (696 mg, 53% yield) as a light brown powder. LCMS m/z=223.0 [M+H]⁺.

### Preparation 11

### 6′-chloro-2,3,5,6-tetrahydrospiro[pyran-4,3′-pyrrolo[3,2-c]pyridin]-2′(1′H)-one

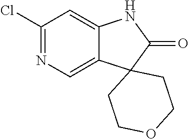 [SMILES: O=C1Nc2cc(Cl)ncc2C12CCOCC2]

6′-Chloro-2,3,5,6-tetrahydrospiro[pyran-4,3′-pyrrolo[3,2-c]pyridin]-2′(1′H)-one was obtained (286 mg, 20.2% yield) as a white powder, from 6-chloro-1,3-dihydro-2H-pyrrolo[3,2-c]pyridin-2-one (Preparation 2) and 1-iodo-2-(2-iodoethoxy)ethane using a similar method to the one described in Preparation 9. LCMS m/z=239.1 [M+H]⁺.

### Preparation 12

### 6′-chloro-2-methyl-1′,2′-dihydrospiro[cyclopropane-1,3′-pyrrolo[3,2-c]pyridine]trifluoroacetate

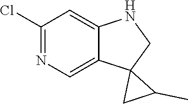

To a suspension of 6′-chloro-2-methylspiro[cyclopropane-1,3′-pyrrolo[3,2-c]pyridin]-2′(1′H)-one

(Preparation 8, 110 mg, 527 μmol) in THF (4 mL) was added LiAlH₄ (2 M, 923 μL) and the reaction mixture was heated to 60° C. for 1 h. The reaction was quenched by dropwise adding aq. potassium sodium tartrate. The mixture was filtered, the filtrate was diluted with EtOAc, washed with aq. NaHCO₃ and brine. The organic layer was separated, dried and concentrated under reduced pressure. The crude was purified by HPLC, eluting with MeCN, water (+0.1% TFA modifier), to give 6′-chloro-2-methyl-1′,2′-dihydrospiro[cyclopropane-1,3′-pyrrolo[3,2-c]pyridine](83 mg, 51% yield). LCMS m/z=195.1 [M+H]⁺.

### Preparation 13

### 6′-chloro-1′,2′-dihydrospiro[cyclobutane-1,3′-pyrrolo[3,2-c]pyridine]

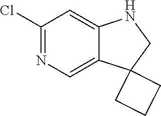 [SMILES: Clc1cc2c(cn1)C1(CCC1)CN2]

To a suspension of 6′-chlorospiro[cyclobutane-1,3′-pyrrolo[3,2-c]pyridin]-2′(1′H)-one (Preparation 9, 530 mg, 2.3 mmol) in THF (20 mL) was added LiAlH₄ (2 M, 3.77 mL). After stirring at rt the reaction mixture was heated to 60° C. for 1 h. The mixture was quenched by dropwise addition of aq. aq. potassium sodium tartrate. The precipitate formed was filtered and the filtrated was diluted with EtOAc. The organic layer was separated, dried and concentrated under reduced pressure. The solid formed was triturated with heptane/ether (1:1) to give 6′-chloro-1′,2′-dihydrospiro[cyclobutane-1,3′-pyrrolo[3,2-c]pyridine](194 mg, 43% yield) as a white solid. LCMS m/z=195.1 [M+H]⁺.

### Preparation 14

### 6′-chloro-1′,2′-dihydrospiro[cyclopentane-1,3′-pyrrolo[3,2-c]pyridine]

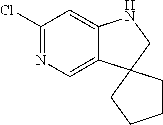 [SMILES: Clc1cc2c(cn1)C1(CCCC1)CN2]

6′-Chloro-1′,2′-dihydrospiro[cyclopentane-1,3′-pyrrolo[3,2-c]pyridine] was prepared from 6′-chlorospiro[cyclopentane-1,3′-pyrrolo[3,2-c]pyridin]-2′(1′H)-one (Preparation 10) using a similar method to the one described in Preparation 13. LCMS m/z=209.1 [M+H]⁺.

### Preparation 15

### 6′-chloro-1′,2,2′,3,5,6-hexahydrospiro[pyran-4,3′-pyrrolo[3,2-c]pyridine]

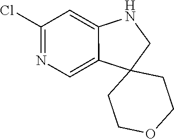 [SMILES: Clc1cc2c(cn1)C1(CCOCC1)CN2]

6′-Chloro-1′,2,2′,3,5,6-hexahydrospiro[pyran-4,3′-pyrrolo[3,2-c]pyridine] was prepared from 6′-chloro-2,3,5,6-tetrahydrospiro[pyran-4,3′-pyrrolo[3,2-c]pyridin]-2′(1′H)-one (Preparation 11) using a similar method to the one described in Preparation 4. LCMS m/z=225.1 [M+H]⁺.

### Preparation 16

### 1-(6-chloro-4-(2-methoxyethoxy)pyridin-2-yl)ethan-1-one

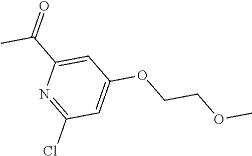

To a solution of 2-chloro-4-(2-methoxyethoxy)pyridine (17 g, 90.6 mmol) in MeCN (100 mL) was added acetaldehyde (5 M, 181 mL), 2-hydroperoxy-2-methyl-propane (16.3 g, 181 mmol), TFA (11.4 g, 99.7 mmol) and FeSO₄ (25.2 g, 90.6 mmol) at 25° C. The reaction mixture was stirred at 80° C. for 24 h under N₂. The mixture was concentrated in vacuo, diluted with H₂O (200 mL) and extracted with EtOAc (150 mL×3). The organic layers were washed with brine (300 mL), dried over Na₂SO₄, filtered, concentrated, then purified by chromatography on silica gel (PE/EtOAc 5/1 to 1/1) to give 1-(6-chloro-4-(2-methoxyethoxy)pyridin-2-yl)ethan-1-one (1.7 g, 8.2% yield) as yellow oil. ¹H NMR: (400 MHz, CDCl₃) δ: ppm 7.51 (d, J=2.4 Hz, 1H), 7.04 (d, J=2.0 Hz, 1H), 4.25-4.22 (m, 2H), 3.82-3.79 (m, 2H), 3.47 (s, 3H), 2.70 (s, 3H).

### Preparation 17

### 4-(benzyloxy)-2-chloro-5-fluoro-6-methylpyrimidine

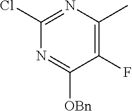 [SMILES: Cc1nc(Cl)nc(OCc2ccccc2)c1F]

A solution of BnOH (5.8 g, 53.9 mmol) and t-BuONa (5.2 g, 53.9 mmol) in toluene (100 mL) was stirred at 0° C. for 10 min. 2,4-Dichloro-5-fluoro-6-methylpyrimidine (9.8 g, 53.9 mmol) was added and the reaction mixture was stirred at 20° C. for 1 h. The mixture was diluted with H₂O (150 mL) and extracted with EtOAc (100 mL×3). The organic layers were washed with brine (200 mL), dried over Na₂SO₄, filtered, concentrated, then purified by chromatography on silica gel (PE/EtOAc 20/1 to 5/1) to give 4-(benzyloxy)-2-chloro-5-fluoro-6-methylpyrimidine (7.9 g, 58.2% yield) as colourless oil. ¹H NMR: (500 MHz, CDCl₃) δ: ppm 7.48-7.46 (m, 2H), 7.42-7.36 (m, 3H), 5.48 (s, 2H), 2.44 (d, J=3.0 Hz, 3H).

### Preparation 18

### methyl 4-(benzyloxy)-5-fluoro-6-methylpyrimidine-2-carboxylate

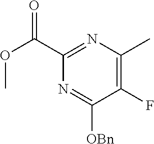 [SMILES: COC(=O)c1nc(C)c(F)c(OCc2ccccc2)n1]

To a solution of 4-(benzyloxy)-2-chloro-5-fluoro-6-methylpyrimidine (Preparation 17, 7.9 g, 31.3 mmol) in MeOH (80 mL) was added TEA (6.4 g, 62.7 mmol) and Pd(dppf)Cl₂ (459 mg, 0.627 mmol). The reaction mixture was stirred at 80° C. for 16 h under CO (50 psi). The mixture was concentrated and purified by chromatography on silica gel (PE/EtOAc 20/1 to 5/1) to give methyl 4-(benzyloxy)-5-fluoro-6-methylpyrimidine-2-carboxylate (3.1 g, 35.8% yield) as yellow oil. ¹H NMR: (500 MHz, CDCl₃) δ: ppm 7.53-7.51 (m, 2H), 7.40-7.35 (m, 3H), 5.58 (s, 2H), 4.03 (s, 3H), 2.55 (d, J=2.8 Hz, 3H).

### Preparation 19

### 1-(4-(benzyloxy)-5-fluoro-6-methylpyrimidin-2-yl)ethan-1-one

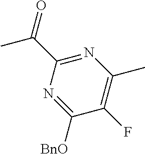 [SMILES: CC(=O)c1nc(C)c(F)c(OCc2ccccc2)n1]

To a solution of methyl 4-(benzyloxy)-5-fluoro-6-methylpyrimidine-2-carboxylate (Preparation 18, 3.1 g, 11.22 mmol) in THF (30 mL) was slowly added dropwise MeMgBr (3 M, 3.8 mL) at −70° C. under N₂ and the mixture was stirred at −70° C. for 1.5 h. The reaction was quenched with sat. aq. NH₄Cl (30.0 mL), treated with H₂O (20 mL) and extracted with EtOAc (40 mL×3). The organic phase was washed with brine (50 mL), dried over Na₂SO₄, filtered, concentrated, then purified by column chromatography (PE/EtOAc=15/1 to 3/1) on silica gel to give 1-(4-(benzyloxy)-5-fluoro-6-methylpyrimidin-2-yl)ethan-1-one (1.6 g, 54.8% yield) as yellow oil. ¹H NMR: (500 MHz, CDCl₃) δ: ppm 7.51-7.49 (m, 2H), 7.40-7.36 (m, 3H), 5.57 (s, 2H), 2.71 (s, 3H), 2.54 (d, J=3.2 Hz, 3H).

### Preparation 20

### 1-(2-chloro-6-methylpyrimidin-4-yl)ethan-1-one

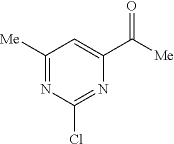 [SMILES: CC(=O)c1cc(C)nc(Cl)n1]

MeMgBr (3M, 1.76 mL, 5.28 mmol) was added to a solution of methyl 2-chloro-6-methylpyrimidine-4-carboxylate (985 mg, 5.28 mmol) in THF (10 mL) at 0° C. under N₂ atmosphere and stirred for 5 h. The reaction mixture was quenched with H₂O (30 mL) and extracted with EtOAc (20 mL×3). The combined organic layers were dried (Na₂SO₄), evaporated to dryness and the residue purified by silica gel chromatography (PE/EtOAc=5/1) to give 1-(2-chloro-6-methylpyrimidin-4-yl)ethan-1-one as a white solid (147 mg, 16%). ¹H NMR (400 MHz, CDCl₃) δ: 7.69 (s, 1H), 2.70 (s, 3H), 2.63 (s, 3H).

### Preparation 21

### 2,4-dichloro-6-(1,1-difluoroethyl)pyridine

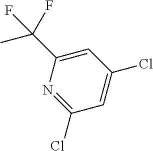

To a solution of 1-(4,6-dichloropyridin-2-yl)ethan-1-one (4.8 g, 25.3 mmol) in DCM (50 mL) was added DAST (20.36 g, 126 mmol) and the reaction mixture was stirred at 25° C. for 12 h. The mixture was added to H₂O (40 mL) dropwise and extracted with DCM (50 mL×3). The combined organic layers were washed with brine (60 mL), dried over Na₂SO₄, filtered and concentrated to give a residue which was purified by chromatography on silica gel (PE) to give 2,4-dichloro-6-(1,1-difluoroethyl)pyridine (4.9 g, 91.5% yield) as a colorless oil. ¹H NMR: (500 MHz, CDCl₃) δ: ppm 7.59 (s, 1H), 7.43 (s, 1H), 2.00 (t, J=18.5 Hz, 3H).

### Preparations 22 to 26

The compounds in the following table were prepared from the corresponding ketone and DAST, using a similar procedure to that described in Preparation 21.

| Preparation | Name, Structure, Starting material, Data |
|---|---|
| 22 | 2,4-dichloro-6-(1, 1-difluoroethyl)pyrimidine |
| | 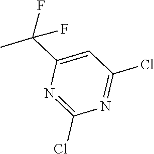 |
| | SM: 1-(2,6-dichloropyrimidin-4-yl)ethan-1-one |
| | 300 mg, 38.4% yield, yellow oil. |
| | ¹H NMR: (500 MHz, CDCl₃) δ: ppm 7.61 (s, 1H), 2.04-1.96 (m, 3H). |
| 23 | 2-chloro-6-(1,1-difluoroethyl)-4-(2-methoxyethoxy)pyridine |
| | 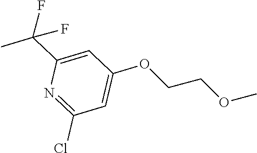 [SMILES: COCCOc1cc(Cl)nc(C(C)(F)F)c1] |
| | SM: 1-(6-chloro-4-(2-methoxyethoxy)pyridin-2-yl)ethan-1-one |
| | (Preparation16) |
| | 70.0 mg, 9.1% yield, colorless oil. |
| | LCMS m/z = 252.1 [M + H]⁺. |
| 24 | 6-bromo-2-(1, 1-difluoroethyl)-3-methoxypyridine |
| | 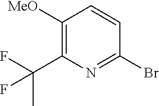 |
| | SM: 1-(6-bromo-3-methoxypyridin-2-yl)ethan-1-one |
| | 130 mg, 59.3% yield, white solid. |
| | LCMS m/z = 253.8 [M + H]⁺ |
| 25 | 4-(benzyloxy)-2-(1,1-difluoroethyl)-5-fluoro-6-methylpyrimidine |
| | 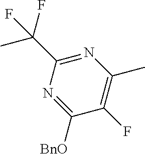 [SMILES: Cc1nc(C(C)(F)F)nc(OCc2ccccc2)c1F] |
| | SM: 1-(4-(benzyloxy)-5-fluoro-6-methylpyrimidin-2-yl)ethan-1-one |
| | (Preparation 19) |
| | 1.6 g, 95.9% yield, yellow oil. |
| | ¹H NMR: (500 MHZ, CDCl₃) δ: ppm 7.48-7.46 (m, 2H), 7.38-7.32 (m, |
| | 3H), 5.51 (s, 2H), 2.48 (d, J = 3.0 Hz, 3H), 1.98 (t, J = 18.0 Hz, 3H). |
| 26 | 2-chloro-4-(1,1-difluoroethyl)-6-methylpyrimidine |
| | 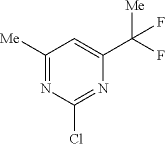 [SMILES: Cc1cc(C(C)(F)F)nc(Cl)n1] |
| | SM: 1-(2-chloro-6-methylpyrimidin-4-yl)ethan-1-one (Preparation 20) |
| | 114 mg, 84.2% yield, yellow oil |
| | ¹H NMR: (500 MHZ, CDCl₃) δ: ppm 7.42 (s, 1H), 2.62 (s, 3H), 1.98 |
| | (t, J = 19.0 Hz, 3H). |

### Preparation 27

### 2-chloro-6-(1,1-difluoroethyl)-4-methoxypyridine

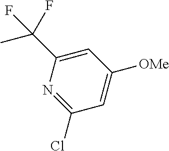

To a solution of 2,4-dichloro-6-(1,1-difluoroethyl)pyridine (Preparation 21, 2 g, 9.43 mmol) in MeOH (20 mL) was added NaOMe (561 mg, 10.4 mmol). The reaction mixture was stirred at 50° C. for 5 h. The mixture was diluted with H₂O (20 mL) and extracted with EtOAc (20 mL×2). The combined organic layers were washed with brine (20 mL×2), dried over Na₂SO₄ and filtered. The filtrate was concentrated under reduced pressure and the residue was purified by chromatography on silica gel (PE/EtOAc 3/1) to give the 2-chloro-6-(1,1-difluoroethyl)-4-methoxypyridine (1.3 g, 66.4% yield) as a yellow solid. ¹H NMR: (500 MHz, CDCl₃) δ: ppm 7.13-7.09 (m, 1H), 6.88-6.87 (m, 1H), 3.90 (s, 3H), 2.02-1.94 (m, 3H).

### Preparation 28

### 2-chloro-4-(1,1-difluoroethyl)-6-methoxypyrimidine

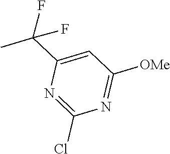

2-Chloro-4-(1,1-difluoroethyl)-6-methoxypyrimidine was obtained from 2,4-dichloro-6-(1,1-difluoroethyl)pyrimidine (Preparation 22) as a yellow oil (110 mg, 65.7% yield) following a similar procedure to that described in Preparation 27. ¹H NMR: (500 MHz, CDCl₃) δ: ppm 6.94 (s, 1H), 4.05 (s, 3H), 1.99-1.91 (m, 3H).

### Preparation 29

### 2-(1,1-difluoroethyl)pyrimidine-4,6-diol

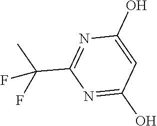

To a solution of malonamide (15.0 g, 147 mmol) in EtOH (500 mL) was added t-BuONa (49.4 g, 514 mmol) and the solution was stirred at 25° C. for 30 mins. Ethyl 2,2-difluoropropanoate (50.7 g, 367 mmol) was added and the reaction stirred under reflux at 100° C. for 16 h. The reaction was cooled, 4N HCl (75 mL) was added and the mixture was concentrated under reduced pressure. The resulting solid was collected and dried to give 2-(1,1-difluoroethyl)pyrimidine-4,6-diol (20.0 g, 77.3% yield) as a yellow solid. LCMS m/z=177.0 [M+H]⁺.

### Preparation 30

### 4,6-dichloro-2-(1,1-difluoroethyl)pyrimidine

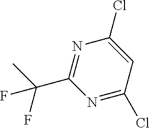

A solution of 2-(1,1-difluoroethyl)pyrimidine-4,6-diol (Preparation 29, 20.0 g, 113.56 mmol) in POCl₃ (200 mL, 2.15 mol) was stirred at 100° C. for 8 h. The mixture was evaporated to dryness, the residue diluted with DCM (150 mL) and added slowly into water. The mixture was extracted with DCM (200 mL×3), the combined organic phase was washed with brine (300 mL), dried over anhydrous Na₂SO₄, filtered and concentrated. The crude was purified by column chromatography (EtOAc in PE 0% to 10%) to give 4,6-dichloro-2-(1,1-difluoroethyl)pyrimidine (23.0 g, 95.1% yield) as a yellow liquid. LCMS m/z=213.0 [M+H]⁺.

### Preparation 31

### 4-chloro-2-(1,1-difluoroethyl)-6-methoxypyrimidine

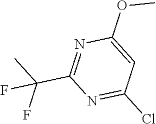

To a solution of 4,6-dichloro-2-(1,1-difluoroethyl)pyrimidine (Preparation 30, 300 mg, 1.41 mmol) in MeOH (5.0 mL) was added NaOMe (76.1 mg, 1.41 mmol) at 0° C. The reaction was stirred at 25° C. for 2 h. The mixture was concentrated under reduced pressure and the residue was purified by chromatography on silica gel (PE/EtOAc 15/1 to 5/1) to give 4-chloro-2-(1,1-difluoroethyl)-6-methoxypyrimidine (260 mg, 88.5% yield) as colourless oil. ¹H NMR: (400 MHz, CDCl₃) δ ppm: 6.81 (s, 1H), 2.02 (t, J=18.5 Hz, 1H).

### Preparation 32

### 4-((1r,3r)-3-(benzyloxy)cyclobutoxy)-2-chloro-6-(1,1-difluoroethyl)pyridine

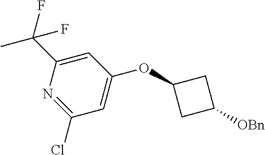 [SMILES: CC(F)(F)c1cc(O[C@H]2C[C@H](OCc3ccccc3)C2)cc(Cl)n1]

A solution of (1r,3r)-3-(benzyloxy)cyclobutan-1-ol in THF (5 mL) and NaH (90.6 mg, 2.26 mmol, 60% purity) was stirred at 25° C. for 0.5 h, then 2,4-dichloro-6-(1,1-difluoroethyl)pyridine (Preparation 21, 400 mg, 1.89 mmol) was added. The reaction mixture was stirred at 25° C. for 5 h. The mixture was treated with H₂O (10 mL) and extracted with EtOAc (10 mL×3). The organic layers were washed with brine (15 mL), dried with Na₂SO₄, filtered, concentrated and purified by chromatography on silica gel (PE/EtOAc 10/1 to 5/1) to give 4-((1r,3r)-3-(benzyloxy)cyclobutoxy)-2-chloro-6-(1,1-difluoroethyl)pyridine (350 mg, 52.4% yield) as yellow oil. ¹H NMR: (400 MHz, CDCl₃) δ: ppm 7.38-7.30 (m, 5H), 7.00 (d, J=2.4 Hz, 1H), 6.75 (d, J=2.0 Hz, 1H), 4.46 (s, 2H), 4.38-4.35 (m, 1H), 3.88-3.82 (m, 1H), 2.93-2.89 (m, 2H), 2.25-2.21 (m, 2H), 1.97 (t, J=18.8 Hz, 3H).

### Preparation 33

### (1r,3r)-3-((2-chloro-6-(1,1-difluoroethyl)pyridin-4-yl)oxy)cyclobutan-1-ol

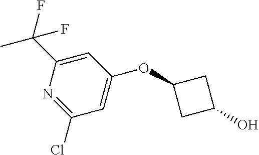 [SMILES: CC(F)(F)c1cc(O[C@H]2C[C@H](O)C2)cc(Cl)n1]

A solution of 4-((1r,3r)-3-(benzyloxy)cyclobutoxy)-2-chloro-6-(1,1-difluoroethyl)pyridine (Preparation 32, 350 mg, 0.989 mmol) in TFA (5 mL) was stirred at 100° C. for 3 h. The mixture was concentrated under reduced pressure and purified by chromatography on silica gel (PE/EtOAc 10/1 to 3/1) to give (1r,3r)-3-((2-chloro-6-(1,1-difluoroethyl)pyridin-4-yl)oxy)cyclobutan-1-ol (250 mg, 95.8% yield) as yellow oil. LCMS m/z=264.0 [M+H]⁺

### Preparation 34

### 2-chloro-6-(1,1-difluoroethyl)-4-((1r,3r)-3-methoxycyclobutoxy)pyridine

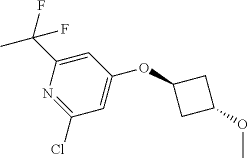 [SMILES: CO[C@H]1C[C@H](Oc2cc(Cl)nc(C(C)(F)F)c2)C1]

To a solution of (1r,3r)-3-((2-chloro-6-(1,1-difluoroethyl)pyridin-4-yl)oxy)cyclobutan-1-ol (Preparation 33, 250 mg, 0.948 mmol) in THF (5 mL) was added NaH (37.9 mg, 0.948 mmol, 60% purity) and MeI (202 mg, 1.42 mmol). The reaction mixture was stirred at 25° C. for 2 h. The mixture was treated with H₂O (20 mL) and extracted with EtOAc (15 mL×3).

The organic layers were washed with brine (20 mL), dried over Na₂SO₄, filtered and concentrated under reduced pressure to give 2-chloro-6-(1,1-difluoroethyl)-4-((1r,3r)-3-methoxycyclobutoxy)pyridine (190 mg, 72.2% yield) as a white solid. ¹H NMR: (500 MHz, CDCl₃) δ: ppm 7.00 (d, J=2.0 Hz, 1H), 6.76 (d, J=2.0 Hz, 1H), 4.42-4.36 (m, 1H), 3.70-3.67 (m, 1H), 3.27 (s, 3H), 2.94-2.91 (m, 2H), 2.18-2.14 (m, 2H), 1.97 (t, J=19.0 Hz, 3H).

### Preparation 35

### 2-(1,1-difluoroethyl)-5-fluoro-6-methylpyrimidin-4-ol

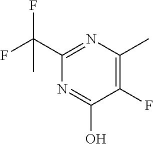

A solution of 4-(benzyloxy)-2-(1,1-difluoroethyl)-5-fluoro-6-methylpyrimidine (Preparation 25, 1.6 g, 5.53 mmol) in TFA (3 mL) was stirred at 25° C. for 16 h followed by 3 h at 100° C. The mixture was concentrated under reduced pressure and purified by chromatography on silica gel (PE/EtOAc 15/1 to 3/1) to give 2-(1,1-difluoroethyl)-5-fluoro-6-methylpyrimidin-4-ol (780 mg, 73.4% yield) as colorless oil. ¹H NMR: (500 MHz, CDCl₃) δ: ppm 2.40 (d, J=3.5 Hz, 3H), 2.02 (t, J=18.5 Hz, 3H).

### Preparation 36

### 4-chloro-2-(1,1-difluoroethyl)-5-fluoro-6-methylpyrimidine

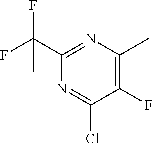

A solution of 2-(1,1-difluoroethyl)-5-fluoro-6-methylpyrimidin-4-ol (Preparation 35, 3.1 g, 16.2 mmol) in POCl₃ (39.5 g, 257 mmol) was stirred at 100° C. for 30 min. The mixture was concentrated under reduced pressure to give the residue which was added to H₂O (15 mL) and extracted with DCM (3 mL×10). The combined organic layers were dried over Na₂SO₄, filtered, concentrated and purified by chromatography on silica gel (PE/EtOAc 15/1 to 3/1) to give 4-chloro-2-(1,1-difluoroethyl)-5-fluoro-6-methylpyrimidine (3.1 g, 90.7% yield) as colorless oil. LCMS m/z=211.0 [M+H]⁺.

### Preparation 37

### Methyl 4-(benzyloxy)-6-methylpyrimidine-2-carboxylate

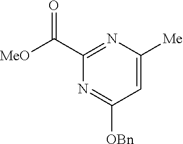 [SMILES: COC(=O)c1nc(C)cc(OCc2ccccc2)n1]

Methyl 4-(benzyloxy)-6-methylpyrimidine-2-carboxylate was obtained, 2.8 g, 56% as a green oil, from 4-(benzyloxy)-2-chloro-6-methylpyrimidine, following a similar procedure to that described in Preparation 18. ¹H NMR (500 MHz, MeOH-d₄) δ: 7.53-7.28 (m, 5H), 6.94 (s, 1H), 5.50 (s, 2H), 4.00 (s, 3H), 2.50 (s, 3H).

### Preparation 38

### 1-(4-(benzyloxy)-6-methylpyrimidin-2-yl)ethan-1-one

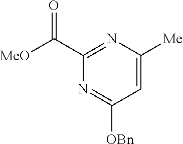 [SMILES: COC(=O)c1nc(C)cc(OCc2ccccc2)n1]

1-(4-(Benzyloxy)-6-methylpyrimidin-2-yl)ethan-1-one was obtained as a yellow oil, 312 mg, 13% yield, from methyl 4-(benzyloxy)-6-methylpyrimidine-2-carboxylate (Preparation 37), following a similar procedure to that described in Preparation 20. LCMS m/z=243.0 [M+H]⁺.

### Preparation 39

### 4-(benzyloxy)-2-(1,1-difluoroethyl)-6-methylpyrimidine

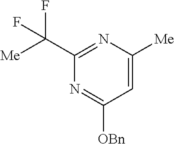 [SMILES: Cc1cc(OCc2ccccc2)nc(C(C)(F)F)n1]

4-(Benzyloxy)-2-(1,1-difluoroethyl)-6-methylpyrimidine was obtained (208 mg, 61.1% yield) as a yellow oil from 1-(4-(benzyloxy)-6-methylpyrimidin-2-yl)ethan-1-one (Preparation 38), following a similar procedure to that described in Preparation 21. LCMS m/z=265.1 [M+H]⁺.

### Preparation 40

### 2-(1,1-difluoroethyl)-6-methylpyrimidin-4-ol

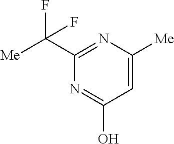 [SMILES: Cc1cc(O)nc(C(C)(F)F)n1]

A mixture of 4-(benzyloxy)-2-(1,1-difluoroethyl)-6-methylpyrimidine (Preparation 39, 208 mg, 0.8 mmol) and TFA (1 mL) was stirred at 100° C. for 12 h. The mixture was concentrated under reduced pressure to give a residue which was purified on silica gel column chromatography (DCM/MeOH 19/1) to give 2-(1,1-difluoroethyl)-6-methylpyrimidin-4-ol (124 mg, 90%) as a yellow solid. ¹H NMR (500 MHz, MeOH-d₄) δ: 6.37 (s, 1H), 2.34 (s, 3H), 1.98 (t, 3H).

### Preparation 41

### 4-chloro-2-(1,1-difluoroethyl)-6-methylpyrimidine

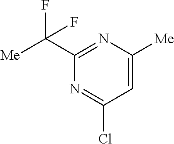 [SMILES: Cc1cc(Cl)nc(C(C)(F)F)n1]

A mixture of 2-(1,1-difluoroethyl)-6-methylpyrimidin-4-ol (Preparation 40, 124 mg, 0.7 mmol) and POCl₃ (1 mL) was stirred at 100° C. for 1 h. The reaction mixture was added to ice water (5 mL) dropwise and extracted with EtOAc (10 mL×3). The combined organics were washed with brine (15 mL), dried (Na₂SO₄) and evaporated to dryness in vacuo to give 4-chloro-2-(1,1-difluoroethyl)-6-methylpyrimidine (86 mg, 63%) as a brown oil. LCMS m/z=193.1 [M+H]⁺.

### Preparation 42

### 4-(benzyloxy)-2-chloro-5-fluoropyrimidine

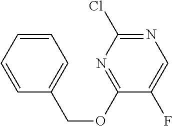 [SMILES: Fc1cnc(Cl)nc1OCc1ccccc1]

A solution of BnOH (6.5 g, 59.9 mmol) and t-BuONa (5.8 g, 59.9 mmol) in toluene (150 mL) was stirred at 0° C. for 10 mins. 2,4-Dichloro-5-fluoropyrimidine (10.0 g, 59.9 mmol) was added slowly and the resulting mixture was stirred at 20° C. for 1 h. The mixture was poured into H₂O (100 mL), extracted with EtOAc (100 mL×3), the combined organic layer was dried over anhydrous Na₂SO₄, filtered and concentrated in vacuo. The residue was purified by chromatography on silica gel (PE/EtOAc 20/1 to 5/1) to give 4-(benzyloxy)-2-chloro-5-fluoropyrimidine (12.5 g, 87.5% yield) as a white solid. ¹H NMR: (500 MHz, CDCl₃) δ: ppm 8.21 (s, 1H), 7.49-7.42 (m, 2H), 7.41-7.38 (m, 3H), 5.51 (s, 2H).

### Preparation 43

### methyl 4-(benzyloxy)-5-fluoropyrimidine-2-carboxylate

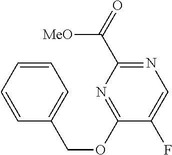 [SMILES: COC(=O)c1ncc(F)c(OCc2ccccc2)n1]

Methyl 4-(benzyloxy)-5-fluoropyrimidine-2-carboxylate was obtained as a white solid, 12 g, 42% yield, from 4-(benzyloxy)-2-chloro-5-fluoropyrimidine (Preparation 42), following a similar procedure to that described in Preparation 18. ¹H NMR: (400 MHz, CDCl₃) δ: ppm 8.44 (d, J=2.0 Hz, 1H), 7.53-7.39 (m, 2H), 7.38-7.35 (m, 3H), 5.61 (s, 2H), 4.03 (s, 3H).

### Preparation 44

### 1-(4-(benzyloxy)-5-fluoropyrimidin-2-yl)ethan-1-one

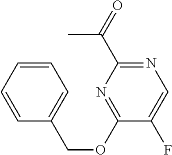 [SMILES: CC(=O)c1ncc(F)c(OCc2ccccc2)n1]

To a solution of methyl 4-(benzyloxy)-5-fluoropyrimidine-2-carboxylate (Preparation 43, 12.0 g, 45.76 mmol) in THF (120 mL) was added dropwise MeMgBr (3 M, 18.3 mL, 54.9 mmol) at −78° C. and the reaction mixture was stirred for 2 h under N₂. The mixture was quenched with sat. NH₄Cl (30 mL) at −78° C., poured into H₂O (60 mL) and extracted with EtOAc (100 mL×3). The combined organic layer was dried over anhydrous Na₂SO₄, filtered and concentrated in vacuo. The residue was purified by chromatography on silica gel (PE/EtOAc 20/1 to 3/1) to give 1-(4-(benzyloxy)-5-fluoropyrimidin-2-yl)ethan-1-one (7.3 g, 64.8% yield) as a white solid. ¹H NMR: (400 MHz, CDCl₃) δ: ppm 8.45 (d, J=2.0 Hz, 1H), 7.52-7.49 (m, 2H), 7.42-7.37 (m, 3H), 5.60 (s, 2H), 2.73 (s, 3H).

### Preparation 45

### 4-(benzyloxy)-2-(1,1-difluoroethyl)-5-fluoropyrimidine

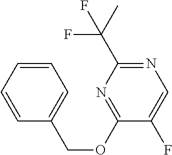 [SMILES: CC(F)(F)c1ncc(F)c(OCc2ccccc2)n1]

4-(Benzyloxy)-2-(1,1-difluoroethyl)-5-fluoropyrimidine was obtained as a colorless oil, 6.7 g, 84% yield, from 1-(4-(benzyloxy)-5-fluoropyrimidin-2-yl)ethan-1-one (Preparation 44) following a similar procedure to that described in Preparation 21. ¹H NMR: (400 MHz, CDCl₃) δ: ppm 8.38 (d, J=2.4 Hz, 1H), 7.51-7.40 (m, 2H), 7.39-7.36 (m, 3H), 5.56 (s, 2H), 2.02 (t, J=18.4 Hz, 3H).

### Preparation 46

### 2-(1,1-difluoroethyl)-5-fluoropyrimidin-4-ol

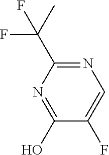

A solution of 4-(benzyloxy)-2-(1,1-difluoroethyl)-5-fluoropyrimidine (Preparation 45, 6.7 g, 24.9 mmol) in TFA (40 mL) was stirred at 100° C. for 12 h. The reaction was concentrated under reduced pressure and the residue was purified by chromatography on silica gel (PE/EtOAc 20/1 to 3/1) to give 2-(1,1-difluoroethyl)-5-fluoropyrimidin-4-ol (3.2 g, 72.1% yield) as a white solid. ¹H NMR: (400 MHz, CDCl₃) δ: ppm 11.61 (br s, 1H), 7.93 (d, J=2.4 Hz, 1H), 2.03 (t, J=18.8 Hz, 3H).

### Preparation 47

### 4-chloro-2-(1,1-difluoroethyl)-5-fluoropyrimidine

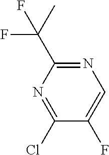

A solution of 2-(1,1-difluoroethyl)-5-fluoropyrimidin-4-ol (Preparation 46, 3.2 g, 18 mmol) in POCl₃ (20.0 mL) was stirred at 100° C. for 2 h. The mixture was slowly poured into ice/water (50 mL) and extracted with EtOAc (60 mL×3). The combined organic phase was washed with brine (50 mL×2), dried over Na₂SO₄ and filtered. The filtrate was concentrated under reduced pressure and the residue was purified by chromatography on silica gel (PE/EtOAc 20/1 to 5/1) to give 4-chloro-2-(1,1-difluoroethyl)-5-fluoropyrimidine (2.3 g, 65.1% yield) as colorless oil. LCMS m/z=197.1 [M+H]⁺

### Preparation 48

### 2-bromo-6-(oxetan-3-yl)pyridine

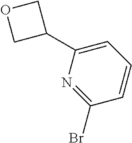 [SMILES: Brc1cccc(C2COC2)n1]

To a solution of 2,6-dibromopyridine (1.0 g, 4.22 mmol) and 3-iodooxetane (1.6 g, 8.44 mmol) in DME (10 mL) were added Ir[dF(CF₃)ppy]₂(dtbbpy)PF₆ (47.4 mg, 0.422 mmol), LiOH (202 mg, 8.44 mmol), NiCl₂·glyme (92.5 mg, 0.422 mmol), tris(trimethylsilyl)silane (1.1 g, 4.22 mmol) and dtbbpy (113 mg, 0.422 mmol). The reaction mixture was stirred and irradiated with blue LEDs for 12 h at 25° C. under N₂. The mixture was concentrated and diluted with H₂O (20 mL) and extracted with EtOAc (20 mL×3). The combined organic layers were washed with brine (20 mL×2), dried over Na₂SO₄ and filtered. The filtrate was concentrated under reduced pressure and the residue was purified by chromatography on silica gel (PE/EtOAc 10/1 to 3/1) to give 2-bromo-6-(oxetan-3-yl)pyridine (250 mg, 27.7% yield) as yellow oil. ¹H NMR: (500 MHz, CDCl₃) δ: ppm 7.54 (t, J=8.0 Hz, 1H), 7.40 (d, J=7.5 Hz, 1H), 7.28 (d, J=7.5 Hz, 1H), 5.05-5.02 (m, 2H), 4.91-4.89 (m, 2H), 4.40-4.35 (m, 1H).

### Preparation 49

### 3-(4-chloropyrimidin-2-yl)oxetan-3-ol

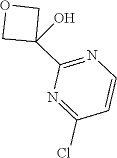 [SMILES: OC1(c2nccc(Cl)n2)COC1]

To a solution of 2-bromo-4-chloropyrimidine (2 g, 10.3 mmol) in toluene (20 mL) was added dropwise n-BuLi (2.5 M, 4.34 mL) at −70° C. for 30 min under N₂ followed by addition of oxetane-3-one (820 mg, 11.4 mmol). The reaction mixture was allowed to warm up to room temperature for 3 h. The mixture was quenched with H₂O (10 mL) and extracted with EtOAc (20 mL×2). The combined organic layers were washed with brine (10 mL×2), dried over Na₂SO₄ and filtered. The filtrate was concentrated under reduced pressure and the residue was purified by chromatography on silica gel (PE/EtOAc 1/1 to 0/1) to give 3-(4-chloropyrimidin-2-yl)oxetan-3-ol (800 mg, 41.5% yield) as a yellow oil. ¹H NMR: (400 MHz, CDCl₃) δ: ppm 8.71 (d, J=5.2 Hz, 1H), 7.36 (d, J=5.2 Hz, 1H), 5.04-5.02 (m, 2H), 4.99-4.97 (m, 2H), 4.92 (s, 1H).

### Preparation 50

### O-(3-(4-chloropyrimidin-2-yl)oxetan-3-yl)S-methyl carbonodithioate

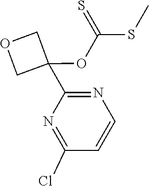 [SMILES: CSC(=S)OC1(c2nccc(Cl)n2)COC1]

To a solution of 3-(4-chloropyrimidin-2-yl)oxetan-3-ol (Preparation 49, 800 mg, 4.29 mmol) in THF (10 mL) was added NaH (514 mg, 12.9 mmol, 60% purity), then MeI (608 mg, 4.29 mmol) and CS₂ (326 mg, 4.29 mmol). The reaction mixture was stirred at 50° C. for 3 h. The reaction was quenched with NH₄Cl (10 mL) and extracted with EtOAc (20 mL×3). The combined organic layers were washed with brine (10 mL×2), dried over Na₂SO₄ and filtered. The filtrate was concentrated under reduced pressure and the residue was purified by chromatography on silica gel (PE/EtOAc 5/1) to give O-(3-(4-chloropyrimidin-2-yl)oxetan-3-yl)S-methyl carbonodithioate (450 mg, 37.9% yield) as a yellow oil. ¹H NMR: (400 MHz, CDCl₃) δ: ppm 8.55 (d, J=5.6 Hz, 1H), 7.21 (d, J=5.2 Hz, 1H), 5.20-5.18 (m, 2H), 5.13-5.11 (m, 2H), 2.59 (s, 3H).

### Preparation 51

### 4-chloro-2-(oxetan-3-yl)pyrimidine

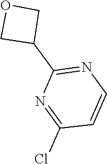 [SMILES: Clc1ccnc(C2COC2)n1]

A mixture of O-(3-(4-chloropyrimidin-2-yl)oxetan-3-yl)S-methyl carbonodithioate (Preparation 50, 450 mg, 1.63 mmol), tributyltin (946 mg, 3.25 mmol) and AIBN (26.7 mg, 163 μmol) in toluene (5 mL) was stirred at 125° C. for 1 h under N₂. The mixture was quenched with KF (5 mL), filtered and extracted with EtOAc (10 mL×3). The combined organic layers were washed with brine (10 mL×2), dried over Na₂SO₄ and filtered. The filtrate was concentrated under reduced pressure and the residue was purified by chromatography on silica gel (Petroleum ether/EtOAc 3/1) to give 4-chloro-2-(oxetan-3-yl)pyrimidine (105 mg, 37.8% yield) as a yellow oil. ¹H NMR: (400 MHz, CDCl₃) δ: ppm 8.63 (d, J=5.6 Hz, 1H), 7.27-7.26 (m, 1H), 5.07-5.01 (m, 4H), 4.52-4.46 (m, 1H).

### Preparation 52

### 3-(6-bromopyridin-2-yl)oxetan-3-ol

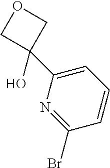 [SMILES: OC1(c2cccc(Br)n2)COC1]

To a solution of 2,6-dibromopyridine (1.0 g, 4.22 mmol) in DCM (20 mL) was added n-BuLi (2.5 M, 1.5 mL) at −70° C. for 30 min followed by oxetane-3-one (304 mg, 4.22 mmol) at −70° C. for 1 h. The mixture was quenched with sat. NH₄Cl (20 mL) and extracted with EtOAc (20 mL×3). The combined organic layers were dried over Na₂SO₄, filtered and concentrated. The residue was purified by chromatography on silica gel (PE/EtOAc 5/1) to give 3-(6-bromopyridin-2-yl)oxetan-3-ol (430 mg, 44.3% yield) as a white solid. ¹H NMR: (400 MHz, CDCl₃) δ: ppm 7.93 (d, J=7.6 Hz, 1H), 7.75-7.71 (m, 1H), 7.50 (d, J=8.0 Hz, 1H), 5.26 (s, 1H), 5.08-5.06 (m, 2H), 4.72-4.69 (m, 2H).

### Preparation 53

### 2-bromo-6-(3-hydroxytetrahydrofuran-3-yl)-4-methyl-pyridine

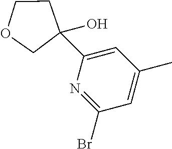

2-Bromo-6-(3-hydroxytetrahydrofuran-3-yl)-4-methyl-pyridine was obtained as a yellow solid, 570 mg, 55.4% yield, from 2,6-dibromo-4-methylpyridine and dihydrofuran-3(2H)-one, following a similar procedure to that described in Preparation 52. LCMS m/z=258.0 [M+H]⁺

### Preparation 54

### 2-bromo-6-(3-methoxyoxetan-3-yl)pyridine

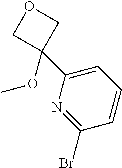

NaH (149 mg, 3.74 mmol, 60% purity) was added to a mixture of 3-(6-bromopyridin-2-yl)oxetan-3-ol (Preparation 52, 430 mg, 1.87 mmol) in THF (8 mL) at 0° C. After 10 min iodomethane (398 mg, 2.81 mmol) was added and the reaction mixture was stirred at 30° C. for 2 h. The mixture was quenched with sat. NH₄Cl (10 mL), poured into H₂O (10 mL) and extracted with EtOAc (20 mL×3). The combined organic layers were dried over Na₂SO₄, filtered, concentrated under reduced pressure and purified by column chromatography on silica gel (PE/EtOAc 5/1) to give 2-bromo-6-(3-methoxyoxetan-3-yl)pyridine (416.3 mg, 91.2% yield) as colorless oil. ¹H NMR: (400 MHz, CDCl₃) δ: ppm 7.61-7.58 (m, 1H), 7.46-7.41 (m, 1H), 7.40-7.39 (m, 1H), 5.05-5.04 (m, 2H), 4.86-4.84 (m, 2H), 3.24 (s, 3H).

### Preparation 55

### 2-fluoro-6-(3-methoxytetrahydrofuran-3-yl)pyridine

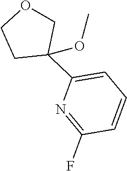

2-Fluoro-6-(3-methoxytetrahydrofuran-3-yl)pyridine was obtained as a colorless oil, 327 mg, 38% from 3-(6-fluoro-2-pyridyl)tetrahydrofuran-3-ol and iodomethane, following a similar procedure to that described in Preparation 54. LCMS m/z=198.1 [M+H]⁺

### Preparation 56

### 2-bromo-6-(3-methoxytetrahydrofuran-3-yl)-4-methyl-pyridine

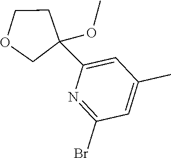

2-Bromo-6-(3-methoxytetrahydrofuran-3-yl)-4-methyl-pyridine was obtained as a brown oil, 500 mg, 94.9% yield, from 2-bromo-6-(3-hydroxytetrahydrofuran-3-yl)-4-methyl-pyridine (Preparation 53) and iodomethane, following a similar procedure to that described in Preparation 54. LCMS m/z=272.1 [M+H]⁺

### Preparation 57

### 2-bromo-6-(3-fluorooxetan-3-yl)pyridine

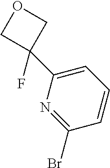 [SMILES: FC1(c2cccc(Br)n2)COC1]

To a solution of 3-(6-bromopyridin-2-yl)oxetan-3-ol (Preparation 52, 680 mg, 2.96 mmol) in DCM (15 mL) was added DAST (715 mg, 4.43 mmol) at 0° C. The reaction mixture was stirred at 25° C. for 4 h. The mixture was quenched with aq. Na₂CO₃ (15 mL), poured into H₂O (20 mL) and extracted with EtOAc (20 mL×2). The combined organic layers were washed with brine (20 mL×2), dried over Na₂SO₄ and filtered. The filtrate was concentrated under reduced pressure and the residue was purified by chromatography on silica gel (PE/EtOAc 3/1) to give 2-bromo-6-(3-fluorooxetan-3-yl)pyridine (493 mg, 71.9% yield) as a white solid. ¹H NMR: (400 MHz, CDCl₃) δ: ppm 7.62-7.58 (m, 1H), 7.49-7.45 (m, 2H), 5.19-5.11 (m, 2H), 5.01-4.94 (m, 2H).

### Preparation 58

### 6′-chloro-1′-(2-(1,1-difluoroethyl)-6-methoxypyrimidin-4-yl)-1′,2′-dihydrospiro[cyclopropane-1,3′-pyrrolo[3,2-c]pyridine]

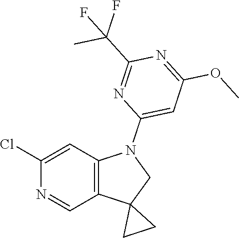 [SMILES: COc1cc(N2CC3(CC3)c3cnc(Cl)cc32)nc(C(C)(F)F)n1]

To a solution of 6′-chloro-1′,2′-dihydrospiro[cyclopropane-1,3′-pyrrolo[3,2-c]pyridine] (Preparation 4, 80.0 mg, 0.443 mmol), 4-chloro-2-(1,1-difluoroethyl)-6-methoxypyrimidine (111 mg, 0.531 mmol) and K₃PO₄ (132 mg, 0.620 mmol) in DMF (2 mL) was added Xantphos (25.7 mg, 0.0443 mmol) and Pd₂(dba)₃ (40.6 mg, 0.0443 mmol). The reaction mixture was stirred at 70° C. for 5 h under N₂. The mixture was diluted with water (60 mL) and extracted with EtOAc (30 mL×3). The combined organic layers were washed with brine (30 mL), dried over Na₂SO₄, filtered and concentrated under vacuum to give the crude product which was purified by chromatography on silica gel (PE/EtOAc 1/0 to 3/1) to give 6′-chloro-1′-(2-(1,1-difluoroethyl)-6-methoxypyrimidin-4-yl)-1′,2′-dihydrospiro[cyclopropane-1,3′-pyrrolo[3,2-c]pyridine](150 mg, 96.3p yield) as a white solid. LCMS m/z=353.0 [M+H]⁺. ¹H NMR: (500 MHz, CDCl₃) δ: ppm 8.32 (s, 1H), 7.58 (s, 1H), 5.90 (s, 1H), 4.03 (s, 3H), 3.99 (s, 2H), 2.07 (t, J=18.5 Hz, 3H), 1.26-1.22 (m, 2H), 1.21-1.19 (m, 2H).

### Preparations 59 to 66

The compounds in the following table were prepared from 6′-chloro-1′,2′-dihydrospiro[cyclopropane-1,3′-pyrrolo[3,2-c]pyridine] (Preparation 4) and the appropriate heterocycle following a similar procedure to that described in Preparation 58.

| Preparation | |
|---|---|
| No | Name, Structure, Starting material (SM), Data |
| 59 ^{A} | 6′-chloro-1′-(6-(oxetan-3-yl)pyridin-2-y1)-1′,2′-dihydrospiro[cyclopropane- |
| Step 2 | 1,3′-pyrrolo[3,2-c]pyridine] |
| | 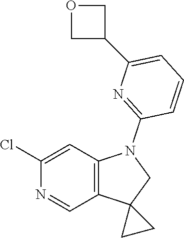 [SMILES: Clc1cc2c(cn1)C1(CC1)CN2c1cccc(C2COC2)n1] |
| | SM: 2-bromo-6-(oxetan-3-yl)pyridine (Preparation 48) |
| | 101 mg, 58.1% yield, white solid. LCMS m/z = 314.1 [M + H]⁺. |
| 60 ^{A} | 6′-chloro-1′-(6-(3-fluorooxetan-3-y1)pyridin-2-yl)-1′,2′- |
| Step 1 | dihydrospiro[cyclopropane-1,3′-pyrrolo[3,2-c]pyridine] |
| | 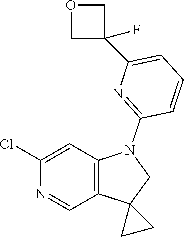 [SMILES: FC1(c2cccc(N3CC4(CC4)c4cnc(Cl)cc43)n2)COC1] |
| | SM: 2-bromo-6-(3-fluorooxetan-3-yl)pyridine (Preparation 57) |
| | 165 mg, 90.1% yield, white solid. |
| | ¹H NMR: (500 MHZ, CDCl₃) δ: ppm 8.37 (s, 1H), 7.75-7.72 (m, 1H), 7.55 |
| | (s, 1H), 7.14-7.12 (m, 1H), 6.69 (d, J = 8.5 Hz, 1H), 5.18-5.06 (m, 4H), 4.15 |
| | (s, 2H), 1.24-1.19 (m, 4H). |
| 61 ^{B, C} | 6′-chloro-1′-(2-(oxetan-3-yl)pyrimidin-4-yl)-1′,2′- |
| Step 1 | dihydrospiro[cyclopropane-1,3′-pyrrolo[3,2-c]pyridine] |
| | 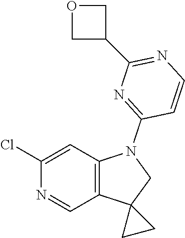 [SMILES: Clc1cc2c(cn1)C1(CC1)CN2c1ccnc(C2COC2)n1] |
| | SM: 4-chloro-2-(oxetan-3-yl)pyrimidine (Preparation 51) |
| | 86.4 mg, 70.8% yield, white solid. |
| | ¹H NMR: (500 MHZ, CDCl₃) δ: ppm 8.55-8.51 (m, 1 H), 8.38 (s, J = 5.0 Hz |
| | 1H), 7.62 (s, 1H), 6.49 (d, J-6.0 Hz, 1H), 5.15-5.12 (m, 2H), 5.10-5.07 (m, |
| | 2H), 4.51-4.47 (m, 1H), 4.12 (s, 2H), 1.29-1.26 (m, 2H), 1.25-1.22 (m, 2H). |
| 62 ^{B, C} | 6′-chloro-1′-(6-(1,1-difluoroethyl)-4-methoxypyridin-2-yl)-1′,2′- |
| Step 5 | dihydrospiro[cyclopropane-1,3′-pyrrolo[3,2-c]pyridine] |
| | 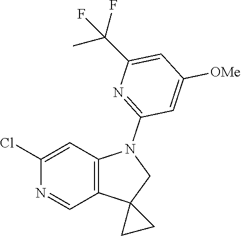 [SMILES: COc1cc(N2CC3(CC3)c3cnc(Cl)cc32)nc(C(C)(F)F)c1] |
| | SM: 2-chloro-6-(1,1-difluoroethyl)-4-methoxypyridine (Preparation 27) |
| | 120 mg, 61.6% yield, yellow solid. LCMS m/z = 352.0 [M + H]⁺. |
| 63 ^{B, C} | 6′-chloro-1′-(6-(1,1-difluoroethyl)-4-(2-methoxyethoxy)pyridin-2-yl)-1′,2′- |
| Step 3 | dihydrospiro[cyclopropane-1,3′-pyrrolo[3,2-c]pyridine] |
| | 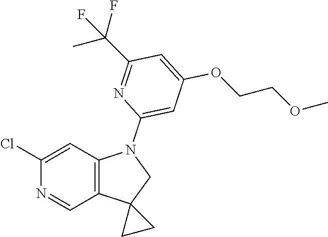 [SMILES: COCCOc1cc(N2CC3(CC3)c3cnc(Cl)cc32)nc(C(C)(F)F)c1] |
| | SM: chloro-6-(1,1-difluoroethyl)-4-(2-methoxyethoxy)pyridine |
| | (Preparation 23) |
| | LCMS m/z = 396.1 [M + H]⁺. |
| 64 | 1-(6-(6′-chlorospiro[cyclopropane-1,3′-pyrrolo[3,2-c]pyridin]-1′(2′H)-yl)-3- |
| Step 1 | fluoropyridin-2-yl)ethan-1-one |
| | 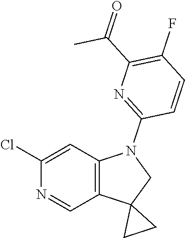 [SMILES: CC(=O)c1nc(N2CC3(CC3)c3cnc(Cl)cc32)ccc1F] |
| | SM: 1-(6-bromo-3-fluoropyridin-2-yl)ethan-1-one |
| | 140.0 mg, 79.6% yield, white solid. LCMS m/z = 318.0 [M + H]⁺. |
| 65° C. | 6′-chloro-1′-(6-(1,1-difluoroethyl)-5-methoxypyridin-2-y1)-1′,2′- |
| Step 3 | dihydrospiro[cyclopropane-1,3′-pyrrolo[3,2-c]pyridine] |
| | 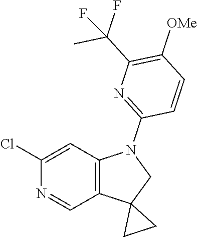 [SMILES: COc1ccc(N2CC3(CC3)c3cnc(Cl)cc32)nc1C(C)(F)F] |
| | 6-bromo-2-(1,1-difluoroethyl)-3-methoxypyridine (Preparation 24) |
| | 140.0 mg, 84.6% yield, white solid. LCMS m/z = 352.0 [M + H]⁺. |
| 66 ^{B, C} | 6′-chloro-1′-(4-(1,1-difluoroethyl)pyrimidin-2-yl)-1′,2,2′,3,5,6- |
| | hexahydrospiro[pyran-4,3′-pyrrolo[3,2-c]pyridine] |
| | 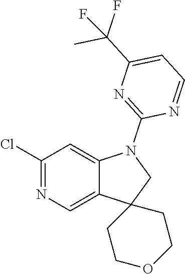 [SMILES: CC(F)(F)c1ccnc(N2CC3(CCOCC3)c3cnc(Cl)cc32)n1] |
| | SM: 6′-chloro-1′,2,2′,3,5,6-hexahydrospiro[pyran-4,3′-pyrrolo[3,2- |
| | c]pyridine] (Preparation 15) |
| | 69 mg, 70% yield, white powder. LCMS m/z = 367.1 [M + H]⁺. |

^{A} K₂CO₃ was used instead of K₃PO₄
^{B} Cs₂CO₃ was used instead of K₃PO₄
^{C} Dioxane was used as the reaction solvent

### Preparation 67

### 6′-chloro-1′-(2-(1,1-difluoroethyl)pyrimidin-4-yl)-1′,2′-dihydrospiro[cyclopentane-1,3′-pyrrolo[3,2-c]pyridine]

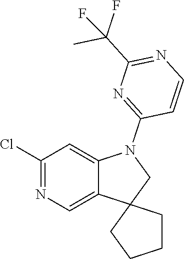 [SMILES: CC(F)(F)c1nccc(N2CC3(CCCC3)c3cnc(Cl)cc32)n1]

A mixture of 6′-chloro-1′,2′-dihydrospiro[cyclopentane-1,3′-pyrrolo[3,2-c]pyridine] (Preparation 14, 40 mg, 192 μmol), 4-chloro-2-(1,1-difluoroethyl)pyrimidine (41 mg, 230 μmol), Xantphos Pd G4 (18 mg, 19 μmol), Cs₂CO₃ (125 mg, 383 μmol) in dioxane (2 mL) was heated at 100° C. for 2 h under N₂. The mixture was filtered, the filtrate was concentrated under reduced pressure and purified by chromatography on silica gel (0-80% EtOAc in heptane) to give 6′-chloro-1′-(2-(1,1-difluoroethyl)pyrimidin-4-yl)-1′,2′-dihydrospiro[cyclopentane-1,3′-pyrrolo[3,2-c]pyridine](38 mg, 56% yield) as a white powder. LCMS m/z=351.1 [M+H]⁺.

### Preparation 68

### tert-butyl 6′-chloro-1′-(4-(1,1-difluoroethyl)pyrimidin-2-yl)-1′,2′-dihydrospiro[piperidine-4,3′-pyrrolo[3,2-c]pyridine]-1-carboxylate

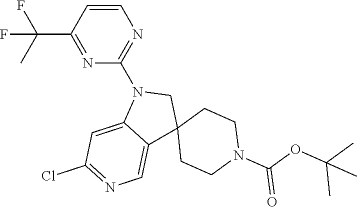 [SMILES: CC(C)(C)OC(=O)N1CCC2(CC1)CN(c1nccc(C(C)(F)F)n1)c1cc(Cl)ncc12]

A mixture of tert-butyl 6′-chloro-1′,2′-dihydrospiro[piperidine-4,3′-pyrrolo[3,2-c]pyridine]-1-carboxylate (200 mg, 618 μmol), 2-chloro-4-(1,1-difluoroethyl)pyrimidine (132 mg, 741 μmol), Cs₂CO₃ (402 mg, 1.24 mmol), Xantphos Pd G4 (59 mg, 62 μmol) in dioxane (6 mL) was heated at 110° C. for 1 h under N₂. The mixture was filtered, the filtrate was concentrated under reduced pressure and purified by chromatography on silica gel (0-80% EtOAc in heptane) to give tert-butyl 6′-chloro-1′-(4-(1,1-difluoroethyl)pyrimidin-2-yl)-1′,2′-dihydrospiro[piperidine-4,3′-pyrrolo[3,2-c]pyridine]-1-carboxylate (116 mg, 40% yield) as a white powder. LCMS m/z=466.2 [M+H]⁺.

### Preparation 69

### tert-butyl 6′-chloro-1′-(2-(1,1-difluoroethyl)pyrimidin-4-yl)-1′,2′-dihydrospiro[piperidine-4,3′-pyrrolo[3,2-c]pyridine]-1-carboxylate

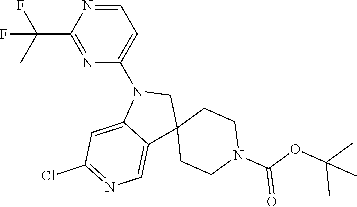 [SMILES: CC(C)(C)OC(=O)N1CCC2(CC1)CN(c1ccnc(C(C)(F)F)n1)c1cc(Cl)ncc12]

tert-Butyl 6′-chloro-1′-(2-(1,1-difluoroethyl)pyrimidin-4-yl)-1′,2′-dihydrospiro[piperidine-4,3′-pyrrolo[3,2-c]pyridine]-1-carboxylate was prepared in a similar method to that described in Preparation 68 from tert-butyl 6′-chloro-1′,2′-dihydrospiro[piperidine-4,3′-pyrrolo[3,2-c]pyridine]-1-carboxylate and 4-chloro-2-(1,1-difluoroethyl)pyrimidine. LCMS m/z=466.2 [M+H]⁺.

### Preparation 70

### tert-butyl 6′-chloro-1′-(6-(1,1-difluoroethyl)pyridin-2-yl)-1′,2′-dihydrospiro[piperidine-4,3′-pyrrolo[3,2-c]pyridine]-1-carboxylate

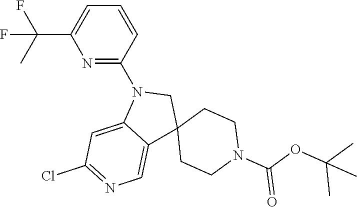 [SMILES: CC(C)(C)OC(=O)N1CCC2(CC1)CN(c1cccc(C(C)(F)F)n1)c1cc(Cl)ncc12]

tert-Butyl 6′-chloro-1′-(6-(1,1-difluoroethyl)pyridin-2-yl)-1′,2′-dihydrospiro[piperidine-4,3′-pyrrolo[3,2-c]pyridine]-1-carboxylate was prepared (252 mg, 88% yield) in a similar method to that described in Preparation 68 from tert-butyl 6′-chloro-1′,2′-dihydrospiro[piperidine-4,3′-pyrrolo[3,2-c]pyridine]-1-carboxylate and 2-bromo-6-(1,1-difluoroethyl)pyridine. LCMS m/z=465.2 [M+H]⁺.

### Preparation 71

### 6′-chloro-1′-(6-chloro-2-(1,1-difluoroethyl)pyrimidin-4-yl)-1′,2′-dihydrospiro[cyclopropane-1,3′-pyrrolo[3,2-c]pyridine]

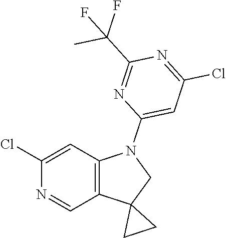 [SMILES: CC(F)(F)c1nc(Cl)cc(N2CC3(CC3)c3cnc(Cl)cc32)n1]

A mixture of 6′-chloro-1′,2′-dihydrospiro[cyclopropane-1,3′-pyrrolo[3,2-c]pyridine] (Preparation 4, 1.1 g, 6.09 mmol), Cs₂CO₃ (3.97 g, 12.2 mmol) and 4,6-dichloro-2-(1,1-difluoroethyl)pyrimidine (Preparation 32, 1.32 g, 6.2 mmol) in DMF (22 mL) was stirred at rt for 1 h. The reaction mixture was diluted with EtOAc and washed with water. The organic layer was separated, dried and concentrated under reduced pressure. The residue was filtered and washed with cold MeOH to give 6′-chloro-1′-(6-chloro-2-(1,1-difluoroethyl)pyrimidin-4-yl)-1′,2′-dihydrospiro[cyclopropane-1,3′-pyrrolo[3,2-c]pyridine](1.86 g, 85% yield). LCMS m/z=357.0 [M+H]⁺.

### Preparation 72

### 6-chloro-1-(6-chloro-2-(1,1-difluoroethyl)pyrimidin-4-yl)-2,3-dihydro-1H-pyrrolo[3,2-c]pyridine

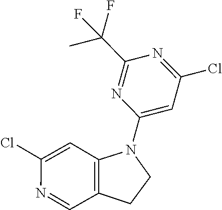 [SMILES: CC(F)(F)c1nc(Cl)cc(N2CCc3cnc(Cl)cc32)n1]

A mixture of 6-chloro-2,3-dihydro-1H-pyrrolo[3,2-c]pyridine (300 mg, 1.94 mmol), Cs₂CO₃ (1.26 g, 3.88 mmol) and 4,6-dichloro-2-(1,1-difluoroethyl)pyrimidine (Preparation 30, 420 mg, 1.97 mmol) in DMF (5 mL) was stirred at rt for 1 h. The reaction mixture was diluted with EtOAc and washed with water (×5). The organic layer was separated, dried and concentrated under reduced pressure. The crude was purified by chromatography on silica gel (0-80% EtOAc in heptane) to give 6-chloro-1-(6-chloro-2-(1,1-difluoroethyl)pyrimidin-4-yl)-2,3-dihydro-1H-pyrrolo[3,2-c]pyridine (358 mg, 56% yield). LCMS m/z=331.0 [M+H]⁺.

### Preparation 73

### 6′-chloro-1′-(2-(1,1-difluoroethyl)-6-methylpyrimidin-4-yl)-1′,2′-dihydrospiro[cyclopropane-1,3′-pyrrolo[3,2-c]pyridine]

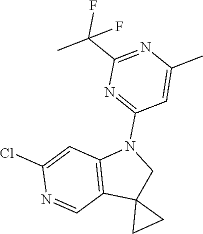 [SMILES: Cc1cc(N2CC3(CC3)c3cnc(Cl)cc32)nc(C(C)(F)F)n1]

A mixture of 4-chloro-2-(1,1-difluoroethyl)-6-methylpyrimidine (Preparation 41, 1.7 g, 8.83 mmol), Cs₂CO₃ (8.6 g, 26.5 mmol) and 6′-chloro-1′,2′-dihydrospiro[cyclopropane-1,3′-pyrrolo[3,2-c]pyridine] (Preparation 4, 1.6 g, 8.83 mmol) in DMF (15 mL) was stirred at 80° C. for 3 h. The mixture was poured into H₂O (50 mL) and extracted with EtOAc (50 mL×3). The organic layers were washed with brine (50 mL×2), dried over Na₂SO₄ and filtered. The filtrate was concentrated and the residue was purified by chromatography on silica gel (PE/EtOAc 20/1 to 3/1) to give 6′-chloro-1′-(2-(1,1-difluoroethyl)-6-methylpyrimidin-4-yl)-1′,2′-dihydrospiro[cyclopropane-1,3′-pyrrolo[3,2-c]pyridine](2.15 g, 72.3% yield) as a yellow solid. ¹H NMR: (400 MHz, CDCl₃) δ: ppm 8.35 (s, 1H), 7.60 (s, 1H), 6.44 (s, 1H), 4.09 (s, 2H), 2.56 (s, 3H), 2.09 (t, J=18.5 Hz, 3H), 1.26-1.21 (m, 4H).

### Preparation 74

### 6′-chloro-1′-(4-(1,1-difluoroethyl)-6-methoxypyrimidin-2-yl)-1′,2′-dihydrospiro[cyclopropane-1,3′-pyrrolo[3,2-c]pyridine]

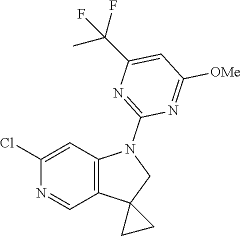 [SMILES: COc1cc(C(C)(F)F)nc(N2CC3(CC3)c3cnc(Cl)cc32)n1]

To a solution of 2-chloro-4-(1,1-difluoroethyl)-6-methoxypyrimidine (Preparation 28, 70 mg, 0.336 mmol) in DMF (2 mL) was added 6′-chloro-1′,2′-dihydrospiro[cyclopropane-1,3′-pyrrolo[3,2-c]pyridine] (Preparation 4, 60.6 mg, 0.336 mmol) and Cs₂CO₃ (219 mg, 0.671 mmol). The reaction mixture was stirred at 100° C. for 16 h. The mixture was concentrated and the residue was purified by chromatography on silica gel (PE/EtOAc 10/1) to give 6′-chloro-1′-(4-(1,1-difluoroethyl)-6-methoxypyrimidin-2-yl)-1′,2′-dihydrospiro[cyclopropane-1,3′-pyrrolo[3,2-c]pyridine](50 mg, 42.2% yield) as a yellow solid. ¹H NMR: (400 MHz, CDCl₃) δ: ppm 8.16 (s, 1H), 7.57 (s, 1H), 6.61 (s, 1H), 4.29 (s, 2H), 4.07 (s, 3H), 2.01-1.92 (m, 3H), 1.20 (s, 4H).

### Preparation 75

### 6′-chloro-1′-(4-(1,1-difluoroethyl)-6-methylpyrimidin-2-yl)-1′,2′-dihydrospiro[cyclopropane-1,3′-pyrrolo[3,2-c]pyridine]

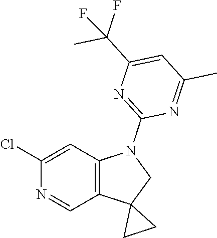 [SMILES: Cc1cc(C(C)(F)F)nc(N2CC3(CC3)c3cnc(Cl)cc32)n1]

6′-Chloro-1′-(4-(1,1-difluoroethyl)-6-methylpyrimidin-2-yl)-1′,2′-dihydrospiro[cyclopropane-1,3′-pyrrolo[3,2-c]pyridine] was obtained from 6′-chloro-1′,2′-dihydrospiro[cyclopropane-1,3′-pyrrolo[3,2-c]pyridine] (Preparation 4) and 2-chloro-4-(1,1-difluoroethyl)-6-methylpyrimidine (Preparation 26) as a yellow solid (167 mg, 89.4% yield) following a similar procedure to that described in Preparation 74. ¹H NMR: (500 MHz, CDCl₃) δ: ppm 8.22 (s, 1H), 7.57 (s, 1H), 7.04 (s, 1H), 4.30 (s, 2H), 2.57 (s, 3H), 2.00 (t, J=19.0 Hz, 3H), 1.19-1.18 (m, 4H).

### Preparation 76

### 6′-chloro-1′-(6-(3-methoxyoxetan-3-yl)pyridin-2-yl)-1′,2′-dihydrospiro[cyclopropane-1,3′-pyrrolo[3,2-c]pyridine]

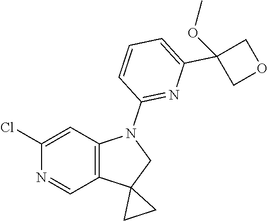 [SMILES: COC1(c2cccc(N3CC4(CC4)c4cnc(Cl)cc43)n2)COC1]

To a mixture of 2-bromo-6-(3-methoxyoxetan-3-yl)pyridine (Preparation 54, 100 mg, 0.55 mmol) and 6′-chloro-1′,2′-dihydrospiro[cyclopropane-1,3′-pyrrolo[3,2-c]pyridine] (Preparation 4, 162 mg, 0.66 mmol) in dioxane (3 mL) was added CuI (21.1 mg, 0.11 mmol), K₂CO₃ (153 mg, 1.11 mmol) and N,N′-dimethylethane-1,2-diamine (19.5 mg, 0.22 mmol). The reaction mixture was stirred at 100° C. for 3 h under N₂. The mixture was concentrated under reduced pressure and the residue was purified by chromatography on silica gel (PE/EtOAc 3/1) to give 6′-chloro-1′-(6-(3-methoxyoxetan-3-yl)pyridin-2-yl)-1′,2′-dihydrospiro[cyclopropane-1,3′-pyrrolo[3,2-c]pyridine](175.2 mg, 92.0% yield) as a white solid. ¹H NMR: (400 MHz, CDCl₃) δ: ppm 8.28 (s, 1H), 7.73 (t, J=8.0 Hz, 1H), 7.53 (s, 1H), 7.08 (d, J=7.6 Hz, 1H), 6.67 (d, J=8.0 Hz, 1H), 5.06 (d, J=6.8 Hz, 2H), 4.96 (d, J=6.8 Hz, 2H), 4.14 (s, 2H), 3.31 (s, 3H), 1.22-1.18 (m, 4H).

### Preparations 77 to 83

The compounds in the following table were prepared from the appropriate pyrrolo[3,2-c]pyridine and the appropriate heterocycle, listed below, following a similar procedure to that described in Preparation 76.
Heterocycle 1: 2-bromo-6-(1,1-difluoroethyl)pyridine
Heterocycle 2: 2-bromo-6-(tetrahydrofuran-3-yl)pyridine

| Preparation | Name, Structure, Starting material, Data |
|---|---|
| 77 | 6′-chloro-1′-(6-(1,1-difluoroethyl)pyridin-2-yl)-1′,2′- |
| | dihydrospiro[cyclopropane-1,3′-pyrrolo[3,2-c]pyridine] |
| | 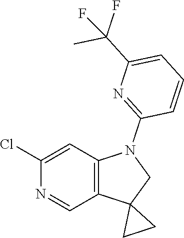 [SMILES: CC(F)(F)c1cccc(N2CC3(CC3)c3cnc(Cl)cc32)n1] |
| | SM: 6′-chloro-1′,2′-dihydrospiro[cyclopropane-1,3′-pyrrolo[3,2-c]pyridine] |
| | (Preparation 4) and Heterocycle 1 |
| | 115 mg, 58% yield, white powder. LCMS m/z = 322.0 [M + H]⁺. |
| 78 | 6′-chloro-1′-(6-(tetrahydrofuran-3-yl)pyridin-2-yl)-1′,2′- |
| | dihydrospiro[cyclopropane-1,3′-pyrrolo[3,2-c]pyridine] |
| | 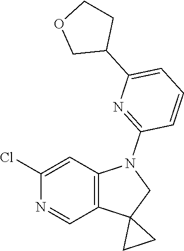 [SMILES: Clc1cc2c(cn1)C1(CC1)CN2c1cccc(C2CCOC2)n1] |
| | SM: 6′-chloro-1′,2′-dihydrospiro[cyclopropane-1,3′-pyrrolo[3,2-c]pyridine] |
| | (Preparation 4) and Heterocycle 2 |
| | LCMS m/z = 328.1 [M + H]⁺. |
| 79 | 6-chloro-1-(6-(1,1-difluoroethyl)pyridin-2-yl)-2,3-dihydro-1H-pyrrolo[3,2- |
| | c]pyridine |
| | 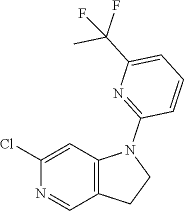 [SMILES: CC(F)(F)c1cccc(N2CCc3cnc(Cl)cc32)n1] |
| | SM: 6-chloro-2,3-dihydro-1H-pyrrolo[3,2-c]pyridine and Heterocycle 1 |
| | 38 mg, 19.9% yield, white powder. |
| | LCMS m/z = 336.0 [M + H]⁺. |
| 80 | 6-chloro-1-(6-(tetrahydrofuran-3-yl)pyridin-2-yl)-2,3-dihydro-1H- |
| | pyrrolo[3,2-c]pyridine |
| | 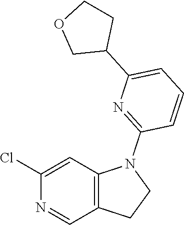 [SMILES: Clc1cc2c(cn1)CCN2c1cccc(C2CCOC2)n1] |
| | SM: 6-chloro-2,3-dihydro-1H-pyrrolo[3,2-c]pyridine and Heterocycle 2 |
| | LCMS m/z = 302.0 [M + H]⁺. |
| 81 | 6′-chloro-1′-(6-(1,1-difluoroethyl)pyridin-2-yl)-1′,2′- |
| | dihydrospiro[cyclobutane-1,3′-pyrrolo[3,2-c]pyridine] |
| | 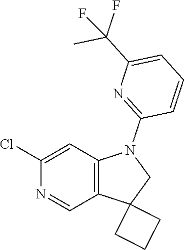 [SMILES: CC(F)(F)c1cccc(N2CC3(CCC3)c3cnc(Cl)cc32)n1] |
| | SM: 6′-chloro-1′,2′-dihydrospiro[cyclobutane-1,3′-pyrrolo[3,2-c]pyridine] |
| | (Preparation 13) and Heterocycle 1. |
| | LCMS m/z = 336.1 [M + H]⁺. |
| 82 | 6′-chloro-1′-(6-(1,1-difluoroethyl)pyridin-2-yl)-1′,2′- |
| | dihydrospiro[cyclopentane-1,3′-pyrrolo[3,2-c]pyridine] |
| | 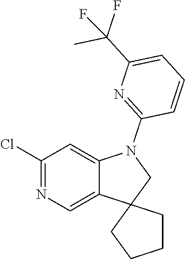 [SMILES: CC(F)(F)c1cccc(N2CC3(CCCC3)c3cnc(Cl)cc32)n1] |
| | SM: 6′-chloro-1′,2′-dihydrospiro[cyclopentane-1,3′-pyrrolo[3,2-c]pyridine] |
| | (Preparation 14) and Heterocycle 1. |
| | LCMS m/z = 350.1 [M + H]⁺. |
| 83 | 6′-chloro-1′-(6-(1,1-difluoroethyl)pyridin-2-yl)-1′,2,2′,3,5,6- |
| | hexahydrospiro[pyran-4,3′-pyrrolo[3,2-c]pyridine] |
| | 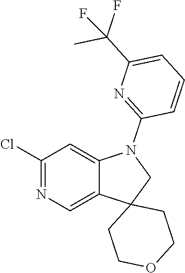 [SMILES: CC(F)(F)c1cccc(N2CC3(CCOCC3)c3cnc(Cl)cc32)n1] |
| | SM: 6′-chloro-1′,2,2′,3,5,6-hexahydrospiro[pyran-4,3′-pyrrolo[3,2- |
| | c]pyridine] (Preparation 15) and Heterocycle 1. |
| | LCMS m/z = 366.1 [M + H]⁺ |

### Preparation 84

### 6-chloro-1-(6-(1,1-difluoroethyl)pyridin-2-yl)-3,3-dimethyl-2,3-dihydro-1H-pyrrolo[3,2-c]pyridine

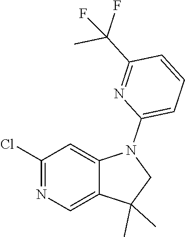 [SMILES: CC(F)(F)c1cccc(N2CC(C)(C)c3cnc(Cl)cc32)n1]

A mixture of 6-chloro-3,3-dimethyl-1,2-dihydropyrrolo[3,2-c]pyridine (100 mg, 547.5 μmol), 2-bromo-6-(1,1-difluoroethyl)pyridine (146 mg, 657 μmol), K₂CO₃ (151 mg, 1.09 mmol), N,N′-dimethylethane-1,2-diamine (9.6 mg, 109 μmol) and CuI (10.4 mg, 54.8 μmol) in dioxane (4 mL) was purged with N₂ and the reaction was then heated at 110° C. for 1 h. The reaction was cooled, filtered and the filtrate was diluted with EtOAc and washed with brine. The organic layer was separated, dried and concentrated in vacuo. The crude was purified by chromatography on silica gel (0-80% EtOAc in heptane) to give 6-chloro-1-(6-(1,1-difluoroethyl)pyridin-2-yl)-3,3-dimethyl-2,3-dihydro-1H-pyrrolo[3,2-c]pyridine (79 mg, 22% yield) as a white powder. LCMS m/z=324.0 [M+H]⁺

### Preparation 85

### 6′-chloro-1′-(2-(1,1-difluoroethyl)pyrimidin-4-yl)-1′,2′-dihydrospiro[cyclopropane-1,3′-pyrrolo[3,2-c]pyridine]

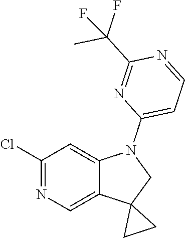 [SMILES: CC(F)(F)c1nccc(N2CC3(CC3)c3cnc(Cl)cc32)n1]

To a solution of 6′-chloro-1′,2′-dihydrospiro[cyclopropane-1,3′-pyrrolo[3,2-c]pyridine] (Preparation 4 45 mg, 249 μmol) in DMF (1 mL) was added NaH (15 mg, 374 μmol, 60% in mineral oil) and the mixture was stirred at rt for 10 min. 4-Chloro-2-(1,1-difluoroethyl)pyrimidine (49 mg, 274 μmol) was added and the reaction was stirred at 60° C. for 2 h. The mixture was diluted with EtOAc and washed with water (×2). The organic layer was separated, dried, and concentrated under reduced pressure. The crude was purified by chromatography on silica gel (0-80% EtOAc in heptane) to give 6-chloro-1-[2-(1,1-difluoroethyl)pyrimidin-4-yl]spiro[2H-pyrrolo[3,2-c]pyridine-3,1′-cyclopropane](78 mg, 97% yield) as a white powder. LCMS m/z=323.1 [M+H]⁺.

### Preparations 86 to 99

The compounds in the following table were prepared from 6′-chloro-1′,2′-dihydrospiro[cyclopropane-1,3′-pyrrolo[3,2-c]pyridine] (Preparation 4) and the appropriate heterocycle listed below, following a similar procedure to that described in Preparation 85.
Heterocycle 3: 2-chloro-4-(1,1-difluoroethyl)pyrimidine
Heterocycle 4: 4-chloro-2-(2-fluoropropan-2-yl)pyrimidine
Heterocycle 5: 2-fluoro-6-(3-methoxytetrahydrofuran-3-yl)pyridine (Preparation 57)
Heterocycle 6: 3-(6-fluoropyridin-2-yl)tetrahydrofuran-3-ol (commercial)

| Preparation | Name, Structure, Starting material (SM), Data |
|---|---|
| 86 | 6′-chloro-1′-(4-(1,1-difluoroethyl)pyrimidin-2-yl)-1′,2′- |
| | dihydrospiro[cyclopropane-1,3′-pyrrolo[3,2-c]pyridine] |
| | 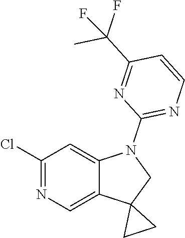 [SMILES: CC(F)(F)c1ccnc(N2CC3(CC3)c3cnc(Cl)cc32)n1] |
| | SM: 6′-chloro-1′,2′-dihydrospiro[cyclopropane-1,3′-pyrrolo[3,2-c]pyridine] |
| | (Preparation 4) and Heterocycle 3 |
| | 41 mg, 51% yield, white powder. LCMS m/z = 323.1 [M + H]⁺. |
| 87 | 6′-chloro-1′-(2-(2-fluoropropan-2-yl)pyrimidin-4-yl)-1′,2′- |
| | dihydrospiro[cyclopropane-1,3′-pyrrolo[3,2-c]pyridine] |
| | 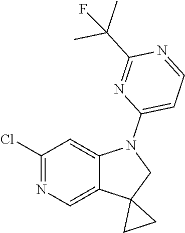 [SMILES: CC(C)(F)c1nccc(N2CC3(CC3)c3cnc(Cl)cc32)n1] |
| | SM: 6′-chloro-1′,2′-dihydrospiro[cyclopropane-1,3′-pyrrolo[3,2-c]pyridine] |
| | (Preparation 4) and Heterocycle 4 |
| | LCMS m/z = 319.0 [M + H]⁺. |
| 88 | 6′-chloro-1′-(6-(3-methoxytetrahydrofuran-3-yl)pyridin-2-yl)-1′,2′- |
| | dihydrospiro[cyclopropane-1,3′-pyrrolo[3,2-c]pyridine] |
| | 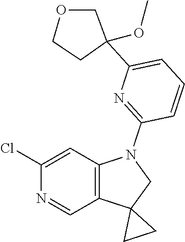 [SMILES: COC1(c2cccc(N3CC4(CC4)c4cnc(Cl)cc43)n2)CCOC1] |
| | SM: 6′-chloro-1′,2′-dihydrospiro[cyclopropane-1,3′-pyrrolo[3,2-c]pyridine] |
| | (Preparation 4) and Heterocycle 5 |
| | 68 mg, 57% yield, white powder. LCMS m/z = 358.1 [M + H]⁺. |
| 89 | 3-(6-(6′-chlorospiro[cyclopropane-1,3′-pyrrolo[3,2-c]pyridin]-1′(2′H)- |
| | yl)pyridin-2-yl)tetrahydrofuran-3-ol |
| | 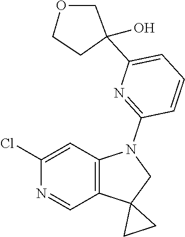 [SMILES: OC1(c2cccc(N3CC4(CC4)c4cnc(Cl)cc43)n2)CCOC1] |
| | SM: 6′-chloro-1′,2′-dihydrospiro[cyclopropane-1,3′-pyrrolo[3,2-c]pyridine] |
| | (Preparation 4) and Heterocycle 6 |
| | LCMS m/z = 344.1 [M + H]⁺. |
| 90 | 6-chloro-1-(2-(1,1-difluoroethyl)pyrimidin-4-yl)-2,3-dihydro-1H- |
| | pyrrolo[3,2-c]pyridine |
| | 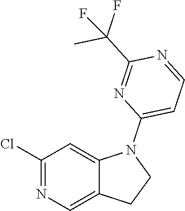 [SMILES: CC(F)(F)c1nccc(N2CCc3cnc(Cl)cc32)n1] |
| | SM: 6-chloro-2,3-dihydro-1H-pyrrolo[3,2-c]pyridine and Heterocycle 4 |
| | 175 mg, 70% yield, white powder. LCMS m/z = 297.0 [M + H]⁺. |
| 91^{A} | 6-chloro-1-(4-(1,1-difluoroethyl)pyrimidin-2-yl)-2,3-dihydro-1H- |
| | pyrrolo[3,2-c]pyridine |
| | 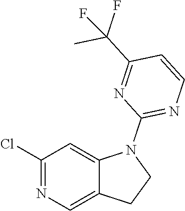 [SMILES: CC(F)(F)c1ccnc(N2CCc3cnc(Cl)cc32)n1] |
| | SM: 6-chloro-2,3-dihydro-1H-pyrrolo[3,2-c]pyridine and Heterocycle 3 |
| | 45 mg, 23% yield, white powder. LCMS m/z = 297.0 [M + H]⁺. |
| 92 | 6-chloro-1-(2-(1,1-difluoroethyl)pyrimidin-4-yl)-3,3-dimethyl-2,3-dihydro- |
| | 1H-pyrrolo[3,2-c]pyridine |
| | 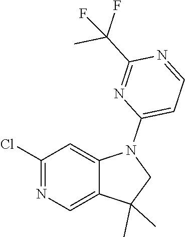 [SMILES: CC(F)(F)c1nccc(N2CC(C)(C)c3cnc(Cl)cc32)n1] |
| | SM: 6-chloro-3,3-dimethyl-1,2-dihydropyrrolo[3,2-c]pyridine and |
| | Heterocycle 4 |
| | 64 mg, 36% yield, white powder. LCMS m/z = 325.0 [M + H]⁺. |
| 93^{A} | 6-chloro-1-(4-(1,1-difluoroethyl)pyrimidin-2-yl)-3,3-dimethyl-2,3-dihydro- |
| | 1H-pyrrolo[3,2-c]pyridine |
| | 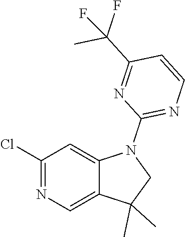 [SMILES: CC(F)(F)c1ccnc(N2CC(C)(C)c3cnc(Cl)cc32)n1] |
| | SM: 6-chloro-3,3-dimethyl-1,2-dihydropyrrolo[3,2-c]pyridine and |
| | Heterocycle 3 |
| | LCMS m/z = 325.0 [M + H]⁺. |
| 94 | 6′-chloro-1′-(4-(1,1-difluoroethyl)pyrimidin-2-yl)-2-methyl-1′,2′- |
| | dihydrospiro[cyclopropane-1,3′-pyrrolo[3,2-c]pyridine] |
| | 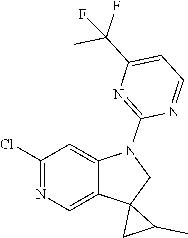 [SMILES: CC1CC12CN(c1nccc(C(C)(F)F)n1)c1cc(Cl)ncc12] |
| | SM: 6′-chloro-2-methyl-1′,2′-dihydrospiro[cyclopropane-1,3′-pyrrolo[3,2- |
| | c]pyridine] trifluoroacetate (Preparation 12) and Heterocycle 3. |
| | LCMS m/z = 337.0 [M + H]⁺. |
| 95 | 6′-chloro-1′-(2-(1,1-difluoroethyl)pyrimidin-4-yl)-2-methyl-1′,2′- |
| | dihydrospiro[cyclopropane-1,3′-pyrrolo[3,2-c]pyridine] |
| | 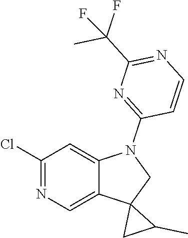 [SMILES: CC1CC12CN(c1ccnc(C(C)(F)F)n1)c1cc(Cl)ncc12] |
| | SM: 6′-chloro-2-methyl-1′,2′-dihydrospiro[cyclopropane-1,3′-pyrrolo[3,2- |
| | c]pyridine] trifluoroacetate (Preparation 12) and Heterocycle 4 |
| | LCMS m/z = 337.0 [M + H]⁺. |
| 96 | 6′-chloro-1′-(4-(1,1-difluoroethyl)pyrimidin-2-yl)-1′,2′- |
| | dihydrospiro[cyclobutane-1,3′-pyrrolo[3,2-c]pyridine] |
| | 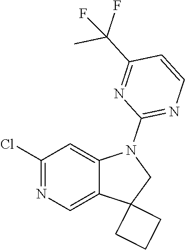 [SMILES: CC(F)(F)c1ccnc(N2CC3(CCC3)c3cnc(Cl)cc32)n1] |
| | SM: 6′-chloro-1′,2′-dihydrospiro[cyclobutane-1,3′-pyrrolo[3,2-c]pyridine] |
| | (Preparation 13) and Heterocycle 3 |
| | LCMS m/z = 337.0 [M + H]⁺. |
| 97 | 6′-chloro-1′-(2-(1,1-difluoroethyl)pyrimidin-4-yl)-1′,2′- |
| | dihydrospiro[cyclobutane-1,3′-pyrrolo[3,2-c]pyridine] |
| | 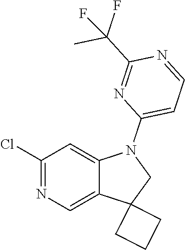 [SMILES: CC(F)(F)c1nccc(N2CC3(CCC3)c3cnc(Cl)cc32)n1] |
| | SM: 6′-chloro-1′,2′-dihydrospiro[cyclobutane-1,3′-pyrrolo[3,2-c]pyridine] |
| | (Preparation 13) and Heterocycle 4 |
| | LCMS m/z = 337.0 [M + H]⁺. |
| 98 | 6′-chloro-1′-(4-(1,1-difluoroethyl)pyrimidin-2-yl)-1′,2′- |
| | dihydrospiro[cyclopentane-1,3′-pyrrolo[3,2-c]pyridine] |
| | 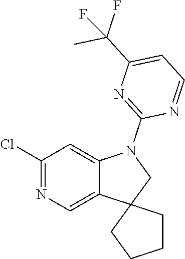 [SMILES: CC(F)(F)c1ccnc(N2CC3(CCCC3)c3cnc(Cl)cc32)n1] |
| | SM: 6′-chloro-1′,2′-dihydrospiro[cyclopentane-1,3′-pyrrolo[3,2-c]pyridine] |
| | (Preparation 14) and Heterocycle 3. |
| | LCMS m/z = 351.1 [M + H]⁺. |
| 99 | 6′-chloro-1′-(2-(1,1-difluoroethyl)pyrimidin-4-yl)-1′,2,2′,3,5,6- |
| | hexahydrospiro[pyran-4,3′-pyrrolo[3,2-c]pyridine] |
| | 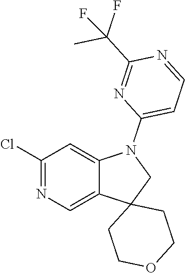 [SMILES: CC(F)(F)c1nccc(N2CC3(CCOCC3)c3cnc(Cl)cc32)n1] |
| | SM: 6′- |
| | chloro-1′,2,2′,3,5,6-hexahydrospiro[pyran-4,3′-pyrrolo[3,2-c]pyridine] |
| | (Preparation 15) and Heterocycle 4. |
| | LCMS m/z = 367.1 [M + H]⁺. |

A = THF was used as the reaction solvent

### Preparation 100

### 2-(6′-chlorospiro[cyclopropane-1,3′-pyrrolo[3,2-c]pyridin]-1′(2′H)-yl)thiazole

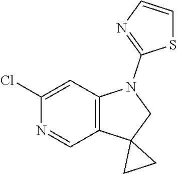 [SMILES: Clc1cc2c(cn1)C1(CC1)CN2c1nccs1]

To a cooled solution of 6′-chloro-1′,2′-dihydrospiro[cyclopropane-1,3′-pyrrolo[3,2-c]pyridine] (Preparation 4, 45 mg, 249.1 μmol) in DMF (2.03 mL) was added NaH (14.95 mg, 373.7 μmol, 60% purity). The solution was stirred at rt for 20 mins, 2-bromothiazole (49.0 mg, 299 μmol) was added and the reaction was stirred at 60° C. overnight. The mixture was diluted with EtOAc, washed with NaHCO₃, H₂O and brine. The organic layer was dried over Na₂SO₄ and the solvent removed in vacuo to give 2-(6′-chlorospiro[cyclopropane-1,3′-pyrrolo[3,2-c]pyridin]-1′(2′H)-yl)thiazole. LCMS m/z=264 [M+H]⁺

### Preparation 101

### 6′-chloro-1′-(6-methylpyrimidin-4-yl)-1′,2′-dihydrospiro[cyclopropane-1,3′-pyrrolo[3,2-c]pyridine]

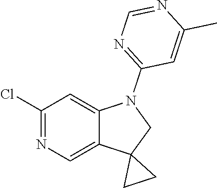 [SMILES: Cc1cc(N2CC3(CC3)c3cnc(Cl)cc32)ncn1]

To a solution of 6′-chloro-1′,2′-dihydrospiro[cyclopropane-1,3′-pyrrolo[3,2-c]pyridine] (Preparation 4, 50 mg, 277 μmol) in DMSO (2 mL) was added KOtBu (62.1 mg, 554 μmol) and 4-chloro-6-methylpyrimidine (71 mg, 553.6 μmol). The reaction mixture was stirred at rt for 20 min. The mixture was diluted with EtOAc, washed with water (×3) and brine. The organic layer was separated, dried and concentrated under reduced pressure to give 6′-chloro-1′-(6-methylpyrimidin-4-yl)-1′,2′-dihydrospiro[cyclopropane-1,3′-pyrrolo[3,2-c]pyridine](81 mg) which was used without further purification. LCMS m/z=273.0 [M+H]⁺.

### Preparation 102 to 120

The compounds in the following table were prepared from 6′-chloro-1′,2′-dihydrospiro[cyclopropane-1,3′-pyrrolo[3,2-c]pyridine] (Preparation 4) and the appropriate heterocycle following the method described in Preparation 101.

| Preparation No | Name, Structure, Heterocycle (Het), Data |
|---|---|
| 102 | 1′-(2-(tert-butyl)-6-methylpyrimidin-4-yl)-6′-chloro-1′,2′- |
| | dihydrospiro[cyclopropane-1,3′-pyrrolo[3,2-c]pyridine] |
| | 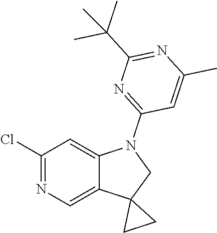 [SMILES: Cc1cc(N2CC3(CC3)c3cnc(Cl)cc32)nc(C(C)(C)C)n1] |
| | Het: 2-(tert-butyl)-4-chloro-6-methylpyrimidine |
| | LCMS m/z = 329.1 [M + H]⁺ |
| 103 | 6′-chloro-1′-(2-cyclopropyl-6-methylpyrimidin-4-yl)-1′,2′- |
| | dihydrospiro[cyclopropane-1,3′-pyrrolo[3,2-c]pyridine] |
| | 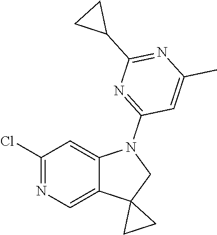 [SMILES: Cc1cc(N2CC3(CC3)c3cnc(Cl)cc32)nc(C2CC2)n1] |
| | Het: 4-chloro-2-cyclopropyl-6-methylpyrimidine |
| | LCMS m/z = 313.1 [M + H]⁺ |
| 104 | 6′-chloro-1′-(2-methoxy-6-methylpyrimidin-4-yl)-1′,2′- |
| | dihydrospiro[cyclopropane-1,3′-pyrrolo[3,2-c]pyridine] |
| | 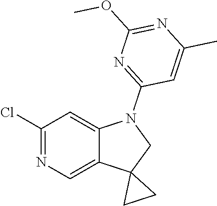 [SMILES: COc1nc(C)cc(N2CC3(CC3)c3cnc(Cl)cc32)n1] |
| | Het: 4-chloro-2-methoxy-6-methylpyrimidine. |
| | LCMS m/z = 303.0 [M + H]⁺ |
| 105 | 6′-chloro-1′-(2-(methoxymethyl)-6-methylpyrimidin-4-yl)-1′,2′- |
| | dihydrospiro[cyclopropane-1,3′-pyrrolo[3,2-c]pyridine] |
| | 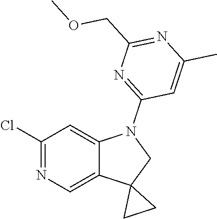 [SMILES: COCc1nc(C)cc(N2CC3(CC3)c3cnc(Cl)cc32)n1] |
| | Het: 4-chloro-2-(methoxymethyl)-6-methylpyrimidine |
| | LCMS m/z = 317.0 [M + H]⁺ |
| 106 | 1′-(2-butyl-6-methylpyrimidin-4-yl)-6′-chloro-1′,2′- |
| | dihydrospiro[cyclopropane-1,3′-pyrrolo[3,2-c]pyridine] |
| | 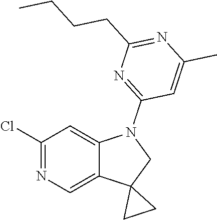 [SMILES: CCCCc1nc(C)cc(N2CC3(CC3)c3cnc(Cl)cc32)n1] |
| | Het: 2-butyl-4-chloro-6-methylpyrimidine |
| | LCMS m/z = 329.1 [M + H]⁺ |
| 107 | 6′-chloro-1′-(2-(ethoxymethyl)-6-methylpyrimidin-4-yl)-1′,2′- |
| | dihydrospiro[cyclopropane-1,3′-pyrrolo[3,2-c]pyridine] |
| | 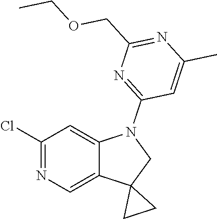 [SMILES: CCOCc1nc(C)cc(N2CC3(CC3)c3cnc(Cl)cc32)n1] |
| | Het: 4-chloro-2-(ethoxymethyl)-6-methylpyrimidine |
| | LCMS m/z = 331.1 [M + H]⁺ |
| 108 | 6′-chloro-1′-(2-cyclobutyl-6-methylpyrimidin-4-yl)-1′,2′- |
| | dihydrospiro[cyclopropane-1,3′-pyrrolo[3,2-c]pyridine] |
| | 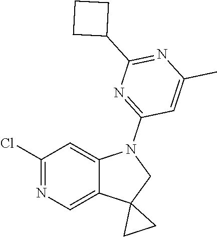 [SMILES: Cc1cc(N2CC3(CC3)c3cnc(Cl)cc32)nc(C2CCC2)n1] |
| | Het: 4-chloro-2-cyclobutyl-6-methylpyrimidine |
| | LCMS m/z = 327.0 [M + H]⁺ |
| 109 | 6′-chloro-1′-(6-methyl-2-(trifluoromethyl)pyrimidin-4-yl)-1′,2′- |
| | dihydrospiro[cyclopropane-1,3′-pyrrolo[3,2-c]pyridine] |
| | 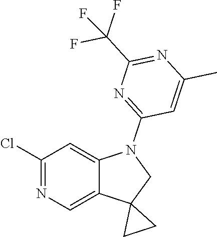 [SMILES: Cc1cc(N2CC3(CC3)c3cnc(Cl)cc32)nc(C(F)(F)F)n1] |
| | Het: 4-chloro-6-methyl-2-(trifluoromethyl)pyrimidine |
| | LCMS m/z = 341.0 [M + H]⁺ |
| 110 | 6′-chloro-1′-(2-(cyclopropylmethyl)-6-methylpyrimidin-4-yl)-1′,2′- |
| | dihydrospiro[cyclopropane-1,3′-pyrrolo[3,2-c]pyridine] |
| | 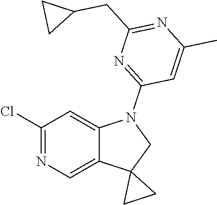 [SMILES: Cc1cc(N2CC3(CC3)c3cnc(Cl)cc32)nc(CC2CC2)n1] |
| | Het: 4-chloro-2-(cyclopropylmethyl)-6-methylpyrimidine |
| | LCMS m/z = 327.0 [M + H]⁺ |
| 111 | 6′-chloro-1′-(2-ethyl-6-methylpyrimidin-4-yl)-1′,2′- |
| | dihydrospiro[cyclopropane-1,3′-pyrrolo[3,2-c]pyridine] |
| | 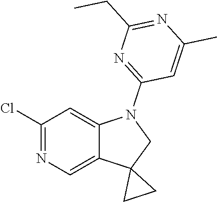 [SMILES: CCc1nc(C)cc(N2CC3(CC3)c3cnc(Cl)cc32)n1] |
| | Het: 4-chloro-2-ethyl-6-methylpyrimidine |
| | LCMS m/z = 301.0 [M + H]⁺ |
| 112 | 6′-chloro-1′-(6-methyl-2-propylpyrimidin-4-yl)-1′,2′- |
| | dihydrospiro[cyclopropane-1,3′-pyrrolo[3,2-c]pyridine] |
| | 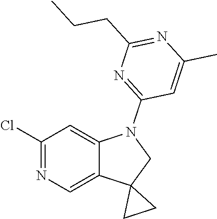 [SMILES: CCCc1nc(C)cc(N2CC3(CC3)c3cnc(Cl)cc32)n1] |
| | Het: 4-chloro-6-methyl-2-propylpyrimidine |
| | LCMS m/z = 315.0 [M + H]⁺ |
| 113 | 6′-chloro-1′-(2-isopropyl-6-methylpyrimidin-4-yl)-1′,2′- |
| | dihydrospiro[cyclopropane-1,3′-pyrrolo[3,2-c]pyridine] |
| | 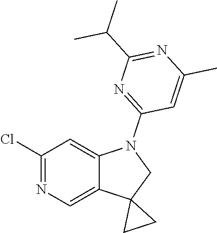 [SMILES: Cc1cc(N2CC3(CC3)c3cnc(Cl)cc32)nc(C(C)C)n1] |
| | Het: 4-chloro-2-isopropyl-6-methylpyrimidine |
| | LCMS m/z = 315.0 [M + H]⁺ |
| 114 | 6′-chloro-1′-(2-(difluoromethyl)-6-methylpyrimidin-4-yl)-1′,2′- |
| | dihydrospiro[cyclopropane-1,3′-pyrrolo[3,2-c]pyridine] |
| | 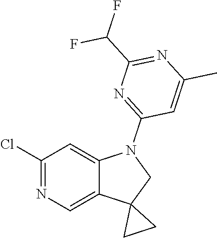 [SMILES: Cc1cc(N2CC3(CC3)c3cnc(Cl)cc32)nc(C(F)F)n1] |
| | Het: 4-chloro-2-(difluoromethyl)-6-methylpyrimidine |
| | LCMS m/z = 323.0 [M + H]⁺ |
| 115 | 6′-chloro-1′-(2,5-dimethylpyrazolo[1,5-a]pyrimidin-7-yl)-1′,2′- |
| | dihydrospiro[cyclopropane-1,3′-pyrrolo[3,2-c]pyridine] |
| | 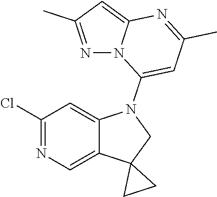 [SMILES: Cc1cc(N2CC3(CC3)c3cnc(Cl)cc32)n2nc(C)cc2n1] |
| | Het: 7-chloro-2,5-dimethylpyrazolo[1,5-a]pyrimidine |
| | LCMS m/z = 326.1 [M + H]⁺. |
| 116 | 6′-chloro-1′-(5-fluoro-2,6-dimethylpyrimidin-4-yl)-1′,2′- |
| | dihydrospiro[cyclopropane-1,3′-pyrrolo[3,2-c]pyridine] |
| | 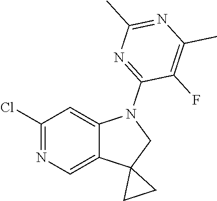 [SMILES: Cc1nc(C)c(F)c(N2CC3(CC3)c3cnc(Cl)cc32)n1] |
| | Het: 4-chloro-5-fluoro-2,6-dimethylpyrimidine |
| | LCMS m/z = 305.0 [M + H]⁺ |
| 117 | 6′-chloro-1′-(2,6-dimethylpyrimidin-4-yl)-1′,2′- |
| | dihydrospiro[cyclopropane-1,3′-pyrrolo[3,2-c]pyridine] |
| | 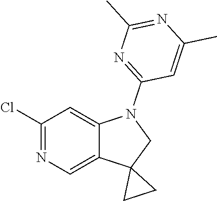 [SMILES: Cc1cc(N2CC3(CC3)c3cnc(Cl)cc32)nc(C)n1] |
| | Het: 4-chloro-2,6-dimethylpyrimidine |
| | LCMS m/z = 287.0 [M + H]⁺ |
| 118 | 6′-chloro-1′-(6-methyl-2-(1-methylcyclopropyl)pyrimidin-4-yl)- |
| | 1′,2′-dihydrospiro[cyclopropane-1,3′-pyrrolo[3,2-c]pyridine] |
| | 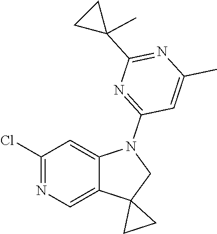 [SMILES: Cc1cc(N2CC3(CC3)c3cnc(Cl)cc32)nc(C2(C)CC2)n1] |
| | Het: 4-chloro-6-methyl-2-(1-methylcyclopropyl)pyrimidine |
| | LCMS m/z = 327.1 [M + H]⁺ |
| 119 | 6′-chloro-1′-(6-methyl-2-(tetrahydro-2H-pyran-3-yl)pyrimidin-4- |
| | yl)-1′,2′-dihydrospiro[cyclopropane-1,3′-pyrrolo[3,2-c]pyridine] |
| | 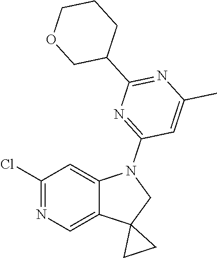 [SMILES: Cc1cc(N2CC3(CC3)c3cnc(Cl)cc32)nc(C2CCCOC2)n1] |
| | Het: 4-chloro-6-methyl-2-(tetrahydro-2H-pyran-3-yl)pyrimidine |
| | LCMS m/z = 357.1 [M + H]⁺ |
| 120 | 6′-chloro-1′-(2-(3-methoxypropyl)-6-methylpyrimidin-4-yl)-1′,2′- |
| | dihydrospiro[cyclopropane-1,3′-pyrrolo[3,2-c]pyridine] |
| | 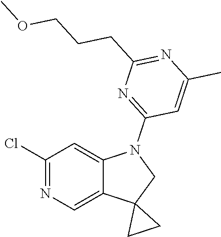 |
| | Het: 4-chloro-2-(3-methoxypropyl)-6-methylpyrimidine |
| | LCMS m/z = 345.1 [M + H]⁺ |

### Preparation 121

### 6′-chloro-1′-(6-methyl-2-(methylthio)pyrimidin-4-yl)-1′,2′-dihydrospiro[cyclopropane-1,3′-pyrrolo[3,2-c]pyridine]

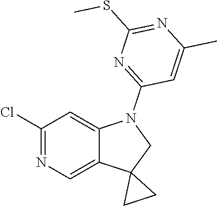 [SMILES: CSc1nc(C)cc(N2CC3(CC3)c3cnc(Cl)cc32)n1]

To a solution of 6′-chloro-1′,2′-dihydrospiro[cyclopropane-1,3′-pyrrolo[3,2-c]pyridine] (Preparation 4, 100 mg, 554 μmol) in THF (5 mL) was added KOtBu (1 M in THF, 1.11 mL) and 4-chloro-6-methyl-2-(methylthio)pyrimidine. The reaction mixture was stirred at rt for 30 min. The mixture was diluted with EtOAc and washed with brine. The organic layer was separated, dried and concentrated, then diluted with EtOAc. The precipitate was filtered and dried to give 80 mg of 6′-chloro-1′-(6-methyl-2-(methylthio)pyrimidin-4-yl)-1′,2′-dihydrospiro[cyclopropane-1,3′-pyrrolo[3,2-c]pyridine] as a light-yellow solid. The mother liquor was evaporated and purified by column chromatography on silica gel (0-80% EtOAc-EtOH 3:1 with 2% NH₄OH in heptane) to give an additional 38 mg of the title compound (total 138 mg, 78% yield) LCMS m/z=319.0 [M+H]⁺.

### Preparation 122

### 2-(6′-chlorospiro[cyclopropane-1,3′-pyrrolo[3,2-c]pyridin]-1′(2′H)-yl)-5-methylthiazole

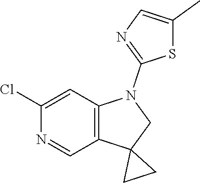 [SMILES: Cc1cnc(N2CC3(CC3)c3cnc(Cl)cc32)s1]

A solution of 6′-chloro-1′,2′-dihydrospiro[cyclopropane-1,3′-pyrrolo[3,2-c]pyridine] (Preparation 4, 60 mg, 332.2 μmol) in THF (2.70 mL) was purged with N₂. KOtBu (1 M, 398.6 μL) was added and the reaction mixture was stirred at rt for 20 min. 2-Bromo-5-methyl-thiazole (70.97 mg, 398.6 μmol) was then added and the reaction was stirred at 60° C. overnight. The mixture was diluted with EtOAc, washed with NaHCO₃, H₂O and brine. The organic layer was dried over Na₂SO₄ and the solvent removed in vacuo to give 2-(6′-chlorospiro[cyclopropane-1,3′-pyrrolo[3,2-c]pyridin]-1′(2′H)-yl)-5-methylthiazole. LCMS m/z=278 [M+H]⁺

### Preparation 123

### 2-(6′-chlorospiro[cyclopropane-1,3′-pyrrolo[3,2-c]pyridin]-1′(2′H)-yl)-4-(1,1-difluoroethyl)thiazole

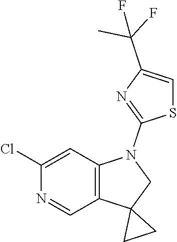 [SMILES: CC(F)(F)c1csc(N2CC3(CC3)c3cnc(Cl)cc32)n1]

2-(6′-Chlorospiro[cyclopropane-1,3′-pyrrolo[3,2-c]pyridin]-1′(2′H)-yl)-4-(1,1-difluoroethyl)thiazole was prepared from 6′-chloro-1′,2′-dihydrospiro[cyclopropane-1,3′ -pyrrolo[3,2-c]pyridine] (Preparation 4) and 2-bromo-4-(1,1,-difluoroethyl)thiazole, following the procedure described in Preparation 122. LCMS m/z=328 [M+H]⁺

### Preparation 124

### 2-(6′-chlorospiro[cyclopropane-1,3′-pyrrolo[3,2-c]pyridin]-1′(2′H)-yl)-4,5-dimethylthiazole

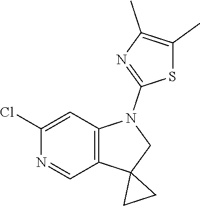 [SMILES: Cc1nc(N2CC3(CC3)c3cnc(Cl)cc32)sc1C]

2-(6′-Chlorospiro[cyclopropane-1,3′-pyrrolo[3,2-c]pyridin]-1′(2′H)-yl)-4,5-dimethylthiazole was prepared from 6′-chloro-1′,2′-dihydrospiro[cyclopropane-1,3′-pyrrolo[3,2-c]pyridine] (Preparation 4) and 2-bromo-4,5-dimethylthiazole, following the procedure described in Preparation 122. LCMS m/z=292 [M+H]⁺

### Preparation 125

### 2-(6′-chlorospiro[cyclopropane-1,3′-pyrrolo[3,2-c]pyridin]-1′(2′H)-yl)-4-(tetrahydro-2H-pyran-3-yl)thiazole

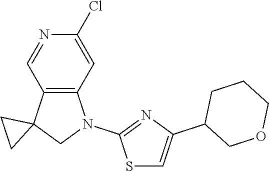 [SMILES: Clc1cc2c(cn1)C1(CC1)CN2c1nc(C2CCCOC2)cs1]

2-(6′-Chlorospiro[cyclopropane-1,3′-pyrrolo[3,2-c]pyridin]-1′(2′H)-yl)-4-(tetrahydro-2H-pyran-3-yl)thiazole was prepared from 6′-chloro-1′,2′-dihydrospiro[cyclopropane-1,3′-pyrrolo[3,2-c]pyridine] (Preparation 4) and 2-bromo-4-(tetrahydro-2H-pyran-3-yl)thiazole (commercial), following the procedure described in Preparation 122. LCMS m/z=348 [M+H]⁺

### Preparation 126

### 2-(6′-chlorospiro[cyclopropane-1,3′-pyrrolo[3,2-c]pyridin]-1′(2′H)-yl)-5-methyl-4-(tetrahydrofuran-3-yl)thiazole

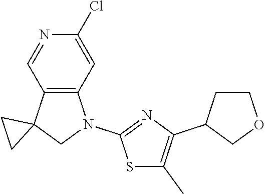 [SMILES: Cc1sc(N2CC3(CC3)c3cnc(Cl)cc32)nc1C1CCOC1]

2-(6′-Chlorospiro[cyclopropane-1,3′-pyrrolo[3,2-c]pyridin]-1′(2′H)-yl)-5-methyl-4-(tetrahydrofuran-3-yl)thiazole was prepared from 6′-chloro-1′,2′-dihydrospiro[cyclopropane-1,3′-pyrrolo[3,2-c]pyridine] (Preparation 4) and 2-bromo-5-methyl-4-(tetrahydrofuran-3-yl)thiazole, following the procedure described in Preparation 122. LCMS m/z=348 [M+H]⁺

### Preparation 127

### 6′-chloro-1′-(2-(1,1-difluoroethyl)-6-(2-methoxyethoxy)pyrimidin-4-yl)-1′,2′-dihydrospiro[cyclopropane-1,3′-pyrrolo[3,2-c]pyridine]

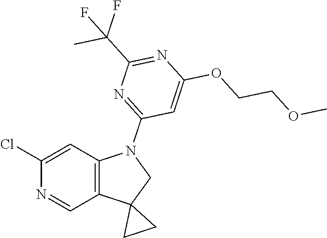 [SMILES: COCCOc1cc(N2CC3(CC3)c3cnc(Cl)cc32)nc(C(C)(F)F)n1]

A solution of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 100 mg, 0.280 mmol), 2-methoxyethan-1-ol (25.6 mg, 0.336 mmol) and Cs₂CO₃ (274 mg, 0.84 mmol) in DMF (5 mL) was stirred at 50° C. for 3 h. The mixture was treated with H₂O (50 mL) and extracted with EtOAc (20 mL×3). The combined organic layers were washed with H₂O (20 mL×3) and brine (30 mL), dried over Na₂SO₄, filtered and concentrated under vacuum to give the crude product which was purified by chromatography on silica gel (PE/EtOAc 1/0 to 1/1) to give 6′-chloro-1′-(2-(1,1-difluoroethyl)-6-(2-methoxyethoxy)pyrimidin-4-yl)-1′,2′-dihydrospiro[cyclopropane-1,3′-pyrrolo[3,2-c]pyridine](60.0 mg, 54.0% yield) as a white solid. ¹H NMR: (400 MHz, CDCl₃) δ: ppm 8.30 (s, 1H), 7.58 (s, 1H), 6.0 (s, 1H), 4.61-4.59 (m, 2H), 4.01 (s, 2H), 3.76-3.73 (m, 2H), 3.44 (s, 3H), 2.05 (t, J=18.5 Hz, 3H), 1.25-1.22 (m, 2H), 1.21-1.17 (m, 2H).

### Preparation 128

### 6′-chloro-1′-(2-(1,1-difluoroethyl)-6-(3-methoxycyclobutoxy)pyrimidin-4-yl)-1′,2′-dihydrospiro[cyclopropane-1,3′-pyrrolo[3,2-c]pyridine]

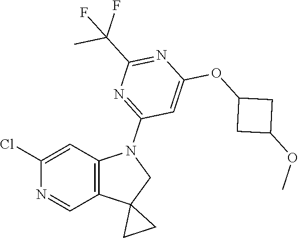 [SMILES: COC1CC(Oc2cc(N3CC4(CC4)c4cnc(Cl)cc43)nc(C(C)(F)F)n2)C1]

6′-Chloro-1′-(2-(1,1-difluoroethyl)-6-(3-methoxycyclobutoxy)pyrimidin-4-yl)-1′,2′-dihydrospiro[cyclopropane-1,3′-pyrrolo[3,2-c]pyridine] was obtained from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and 3-methoxycyclobutan-1-ol as a white solid (80.0 mg, 52.0% yield) following a similar procedure to that described in Preparation 127. LCMS m/z=423.1 [M+H]⁺.

### Preparation 129

### 6′-chloro-1′-(6-cyclopropoxy-2-(1,1-difluoroethyl)pyrimidin-4-yl)-1′,2′-dihydrospiro[cyclopropane-1,3′-pyrrolo[3,2-c]pyridine]

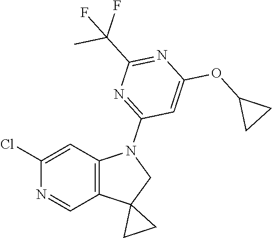 [SMILES: CC(F)(F)c1nc(OC2CC2)cc(N2CC3(CC3)c3cnc(Cl)cc32)n1]

6′-Chloro-1′-(6-cyclopropoxy-2-(1,1-difluoroethyl)pyrimidin-4-yl)-1′,2′-dihydrospiro[cyclopropane-1,3′-pyrrolo[3,2-c]pyridine] was obtained from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and cyclopropanol as a white solid (80.0 mg, 68.6% yield) following a similar procedure to that described in Preparation 127. ¹H NMR: (400 MHz, CDCl₃) δ: ppm 8.36 (s, 1H), 7.62 (s, 1H), 5.99 (s, 1H), 4.39-4.34 (m, 1H), 4.07 (s, 2H), 2.10 (t, J=18.4 Hz, 3H), 1.29-1.27 (m, 2H), 1.24-1.22 (m, 2H), 0.88-0.86 (m, 2H), 0.86-0.84 (m, 2H).

### Preparation 130

### 6-(6′-chlorospiro[cyclopropane-1,3′-pyrrolo[3,2-c]pyridin]-1′(2′H)-yl)-2-(1,1-difluoroethyl)-N,N-dimethylpyrimidin-4-amine

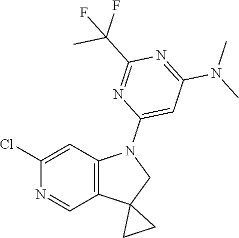 [SMILES: CN(C)c1cc(N2CC3(CC3)c3cnc(Cl)cc32)nc(C(C)(F)F)n1]

A mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 50 mg, 140 μmol) and N-methylmethanamine (2 M in THF, 2 mL) was stirred at rt for 1 h. The solvent was evaporated and the residue diluted with EtOAc, washed with water and brine. The organic layer was dried and concentrated to give 6-(6′-chlorospiro[cyclopropane-1,3′-pyrrolo[3,2-c]pyridin]-1′(2′H)-yl)-2-(1,1-difluoroethyl)-N,N-dimethylpyrimidin-4-amine (49 mg, 95.7% yield) as a white powder which was used without further purification. LCMS m/z=366.1 [M+H]⁺.

### Preparation 131

### 6-(6′-chlorospiro[cyclopropane-1,3′-pyrrolo[3,2-c]pyridin]-1′(2′H)-yl)-2-(1,1-difluoroethyl)-N-methylpyrimidin-4-amine

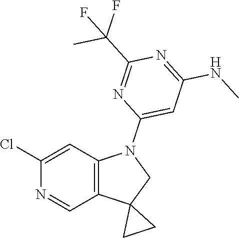 [SMILES: CNc1cc(N2CC3(CC3)c3cnc(Cl)cc32)nc(C(C)(F)F)n1]

A mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 50 mg, 139.98 μmol) and methanamine (2 M, 2.0 mL) was stirred at rt overnight. The solvent was removed, the crude was diluted with EtOAc, washed with water, then brine. The organic layer was dried, filtered, and evaporated under reduced pressure to give 6-(6′-chlorospiro[cyclopropane-1,3′-pyrrolo[3,2-c]pyridin]-1′(2′H)-yl)-2-(1,1-difluoroethyl)-N-methylpyrimidin-4-amine, as a white powder. LCMS m/z=352.1 [M+H]⁺; ¹H NMR (400 MHz, CDCl₃) δ 8.32 (s, 1H), 7.57 (s, 1H), 5.42 (s, 1H), 5.40-5.20 (m, 1H), 4.06 (s, 2H), 2.97 (d, 3H, J=5.0 Hz), 2.05 (t, 3H, J=18.6 Hz), 1.30-1.20 (m, 4H)

### Preparations 132 to 135

The compounds in the following table were prepared from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and the appropriate amine, following the procedure described in Preparation 131.

| Preparation | |
|---|---|
| No | Name, Structure, Amine, Data |
| 132 | 6-(6′-chlorospiro[cyclopropane-1,3′-pyrrolo[3,2-c]pyridin]-1′(2′H)-yl)-2- |
| | (1,1-difluoroethyl)-N-(2-methoxyethyl)pyrimidin-4-amine |
| | 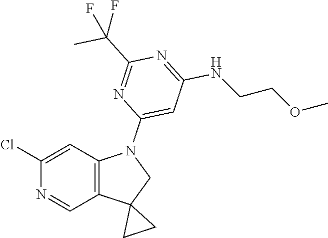 [SMILES: COCCNc1cc(N2CC3(CC3)c3cnc(Cl)cc32)nc(C(C)(F)F)n1] |
| | Amine: 2-methoxyethanamine |
| | LCMS m/z = 396.2 [M + H]⁺; ¹H NMR (400 MHz, CDCl₃) δ 8.31 (s, 1H), |
| | 7.57 (s, 1H), 5.52 (s, 1H), 4.04 (s, 2H), 3.60-3.50 (m, 4H), 3.40 (s, 3H), |
| | 2.04 (t, 3H, J = 18.6 Hz), 1.22 (dd, 4H, J = 3.8, 15.3 Hz) |
| 133 | 6-(6′-chlorospiro[cyclopropane-1,3′-pyrrolo[3,2-c]pyridin]-1′(2′H)-yl)-2- |
| | (1,1-difluoroethyl)-N-(2-methoxyethyl)-N-methylpyrimidin-4-amine |
| | 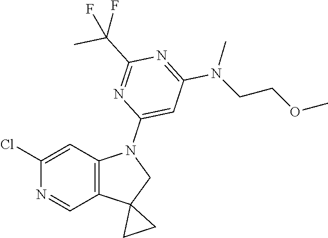 [SMILES: COCCN(C)c1cc(N2CC3(CC3)c3cnc(Cl)cc32)nc(C(C)(F)F)n1] |
| | Amine: 2-methoxy-N-methyl-ethanamine. |
| | LCMS m/z = 410.2 [M + H]⁺ ¹H NMR (400 MHz, CDCl₃) δ 8.33 (s, 1H), |
| | 7.54 (s, 1H), 5.52 (s, 1H), 4.04 (s, 2H), 3.81 (br s, 2H), 3.61 (t, 2H, J = 5.4 |
| | Hz), 3.36 (s, 3H), 3.13 (s, 3H), 2.03 (t, 3H, J = 18.4 Hz), 1.30-1.20 (m, 4H). |
| 134 | 1′-(6-(azetidin-1-yl)-2-(1,1-difluoroethyl)pyrimidin-4-yl)-6′-chloro-1′,2′- |
| | dihydrospiro[cyclopropane-1,3′-pyrrolo[3,2-c]pyridine |
| | 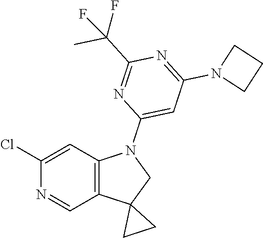 [SMILES: CC(F)(F)c1nc(N2CCC2)cc(N2CC3(CC3)c3cnc(Cl)cc32)n1] |
| | Amine: azetidine. |
| | LCMS m/z = 378.1 [M + H]⁺; ¹H NMR (400 MHz, CDCl₃) δ 8.40-8.30 (m, |
| | 1H), 7.55 (s, 1H), 5.19 (s, 1H), 4.13 (br t, 4H, J = 7.4 Hz), 4.01 (s, 2H), |
| | 2.50-2.40 (m, 2H), 2.03 (t, 3H, J = 18.4 Hz), 1.30-1.20 (m, 4H). |
| 135 | 4-(6-(6′-chlorospiro[cyclopropane-1,3′-pyrrolo[3,2-c]pyridin]-1′(2′H)-yl)- |
| | 2-(1,1-difluoroethyl)pyrimidin-4-yl)morpholine |
| | 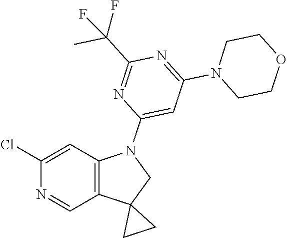 [SMILES: CC(F)(F)c1nc(N2CCOCC2)cc(N2CC3(CC3)c3cnc(Cl)cc32)n1] |
| | Amine: morpholine |
| | LCMS m/z = 408.1 [M +H]⁺ |

### Preparation 136

### 6′-chloro-1′-(6-(1,1-difluoroethyl)-5-fluoropyridin-2-yl)-1′,2′-dihydrospiro[cyclopropane-1,3′-pyrrolo[3,2-c]pyridine]

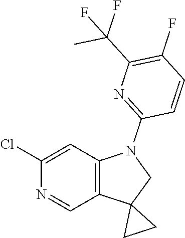 [SMILES: CC(F)(F)c1nc(N2CC3(CC3)c3cnc(Cl)cc32)ccc1F]

A solution of 1-(6-(6′-chlorospiro[cyclopropane-1,3′-pyrrolo[3,2-c]pyridin]-1′(2′H)-yl)-3-fluoropyridin-2-yl)ethan-1-one (Preparation 64, 120.0 mg, 0.378 mmol) in DAST (1 mL) was stirred at 60° C. for 4 h. The mixture was diluted with DCM (30 mL) and treated with H₂O (50 mL). The organic layers were washed with brine (20 mL), dried over Na₂SO₄, filtered and concentrated under vacuum to give the crude product which was purified by chromatography on silica gel (PE/EtOAc 1/0 to 1/1) to give 6′-chloro-1′-(6-(1,1-difluoroethyl)-5-fluoropyridin-2-yl)-1′,2′-dihydrospiro[cyclopropane-1,3′-pyrrolo[3,2-c]pyridine](80.0 mg, 62.3% yield) as a white solid. ¹H NMR: (400 MHz, CDCl₃) δ: ppm 8.02 (s, 1H), 7.50-7.44 (m, 2H), 6.72-6.68 (m, 1H), 4.03 (s, 2H), 2.07 (t, J=19.6 Hz, 3H), 1.17-1.15 (m, 2H), 1.13-1.11 (m, 2H).

### Preparation 137

### 6′-chloro-1′-(2-(1,1-difluoroethyl)-6-ethylpyrimidin-4-yl)-1′,2′-dihydrospiro[cyclopropane-1,3′-pyrrolo[3,2-c]pyridine]

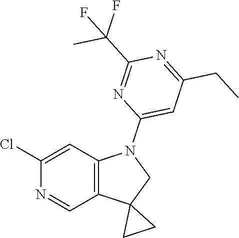 [SMILES: CCc1cc(N2CC3(CC3)c3cnc(Cl)cc32)nc(C(C)(F)F)n1]

To a mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 50 mg, 140 μmol) and Pd(DPEPhos)Cl₂ (1.0 mg, 1.40 μmol) in THF (2 mL) was added ethylzinc bromide (0.5 M, 336 μL) under N₂. The reaction mixture was stirred at rt overnight. The mixture was treated with water and evaporated to dryness. The residue was dry loaded on silica gel and purified by chromatography on silica gel (0-60% EtOAc in heptane) to give 6′-chloro-1′-(2-(1,1-difluoroethyl)-6-ethylpyrimidin-4-yl)-1′,2′-dihydrospiro[cyclopropane-1,3′-pyrrolo[3,2-c]pyridine](38 mg, 77% yield). LCMS m/z=351.1 [M+H]⁺. ¹H NMR: (400 MHz, CDCl₃) δ: ppm 8.39 (s, 1H), 7.62 (s, 1H), 6.45 (s, 1H), 4.13 (s, 2H), 2.83 (q, 2H, J=7.7 Hz), 2.10 (t, 3H, J=18.4 Hz), 1.34 (t, 3H, J=7.7 Hz), 1.30-1.20 (m, 4H).

### Preparation 138

### 6′-chloro-1′-(2-(1,1-difluoroethyl)-6-isopropylpyrimidin-4-yl)-1′,2′-dihydrospiro[cyclopropane-1,3′-pyrrolo[3,2-c]pyridine]

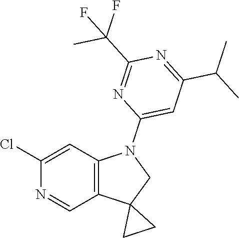 [SMILES: CC(C)c1cc(N2CC3(CC3)c3cnc(Cl)cc32)nc(C(C)(F)F)n1]

6′-Chloro-1′-(2-(1,1-difluoroethyl)-6-isopropylpyrimidin-4-yl)-1′,2′-dihydrospiro[cyclopropane-1,3′-pyrrolo[3,2-c]pyridine] was prepared in an analogous manner of that of Preparation 137 from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and bromo(isopropyl)zinc. LCMS m/z=365.1 [M+H]⁺

### Preparation 139

### 6′-chloro-1′-(6-cyclopropyl-2-(1,1-difluoroethyl)pyrimidin-4-yl)-1′,2′-dihydrospiro[cyclopropane-1,3′-pyrrolo[3,2-c]pyridine]

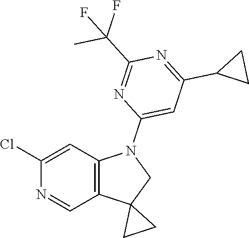 [SMILES: CC(F)(F)c1nc(C2CC2)cc(N2CC3(CC3)c3cnc(Cl)cc32)n1]

6′-Chloro-1′-(6-cyclopropyl-2-(1,1-difluoroethyl)pyrimidin-4-yl)-1′,2′-dihydrospiro[cyclopropane-1,3′-pyrrolo[3,2-c]pyridine] was prepared using a similar method described in Preparation 137 from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and cyclopropylzinc bromide. LCMS m/z=363.1 [M+H]⁺.

### Preparation 140

### 6′-chloro-1′-(6-cyclobutyl-2-(1,1-difluoroethyl)pyrimidin-4-yl)-1′,2′-dihydrospiro[cyclopropane-1,3′-pyrrolo[3,2-c]pyridine]

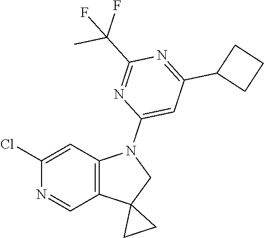 [SMILES: CC(F)(F)c1nc(C2CCC2)cc(N2CC3(CC3)c3cnc(Cl)cc32)n1]

6′-Chloro-1′-(6-cyclobutyl-2-(1,1-difluoroethyl)pyrimidin-4-yl)-1′,2′-dihydrospiro[cyclopropane-1,3′-pyrrolo[3,2-c]pyridine] was prepared using a similar method described in Preparation 137 from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71) and cyclobutylzinc bromide. LCMS m/z=377.2 [M+H]⁺.

### Preparation 141

### 6′-chloro-1′-(2-(1,1-difluoroethyl)-6-(oxetan-3-yl)pyrimidin-4-yl)-1′,2′-dihydrospiro[cyclopropane-1,3′-pyrrolo[3,2-c]pyridine]

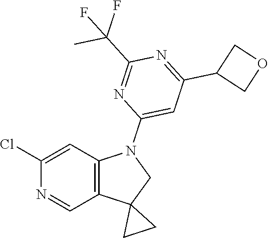 [SMILES: CC(F)(F)c1nc(C2COC2)cc(N2CC3(CC3)c3cnc(Cl)cc32)n1]

To a mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 100 mg, 280 μmol), 3-bromooxetane (38 mg, 280 μmol), tris(trimethylsilyl)silane (69.6 mg, 280 μmol), LiOH (13.4 mg, 560 μmol) was added DME (2 mL) under N₂. In a separate vial, DME (2 mL) was added to dtbbpy (7.5 mg, 28 μmol) and Ir[dF(CF₃)ppy]2(dtbpy)PF₆ (3.1 mg, 2.8 μmol) under N₂. The catalyst mixture was sonicated and added to the reaction vial. The reaction mixture was stirred and irradiated with blue LED at rt overnight. The mixture was concentrated under vacuum, diluted with water and extracted with EtOAc (×3). The combined organic layers were washed with brine, dried and filtered. The filtrate was concentrated under reduced pressure and purified by chromatography on silica gel (0-80% EtOAc in heptane) to give 6′-chloro-1′-(2-(1,1-difluoroethyl)-6-(oxetan-3-yl)pyrimidin-4-yl)-1′,2′-dihydrospiro[cyclopropane-1,3′-pyrrolo[3,2-c]pyridine](11 mg, 10.4% yield). LCMS m/z=379.2 [M+H]⁺.

### Preparations 142 to 144

The compounds in the following table were prepared from 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 73) and the corresponding halide following a similar procedure to that described in Preparation 147.

| Preparation | Name, Structure, Starting material (SM), Data |
|---|---|
| 142 | 6′-chloro-1′-(2-(1,1-difluoroethyl)-6-(3-methoxycyclobutyl)pyrimidin-4-yl)- |
| | 1′,2′-dihydrospiro[cyclopropane-1,3′-pyrrolo[3,2-c]pyridine] |
| | 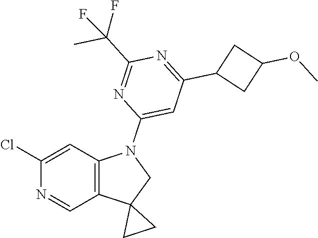 [SMILES: COC1CC(c2cc(N3CC4(CC4)c4cnc(Cl)cc43)nc(C(C)(F)F)n2)C1] |
| | SM: 1-bromo-3-methoxycyclobutane |
| | LCMS m/z = 407.2 [M + H]⁺. |
| 143 | 3-(6-(6′-chlorospiro[cyclopropane-1,3′-pyrrolo[3,2-c]pyridin]-1′(2′H)-yl)-2- |
| | (1,1-difluoroethyl)pyrimidin-4-yl)cyclobutane-1-carbonitrile |
| | 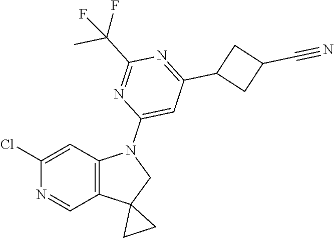 [SMILES: CC(F)(F)c1nc(C2CC(C#N)C2)cc(N2CC3(CC3)c3cnc(Cl)cc32)n1] |
| | SM: 3-bromocyclobutane-1-carbonitrile |
| | LCMS m/z = 402.2 [M + H]⁺. |
| 144 | 6′-chloro-1′-(2-(1,1-difluoroethyl)-6-(tetrahydrofuran-3-yl)pyrimidin-4-yl)- |
| | 1′,2′-dihydrospiro[cyclopropane-1,3′-pyrrolo[3,2-c]pyridine] |
| | 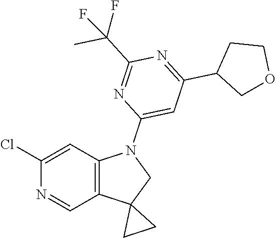 [SMILES: CC(F)(F)c1nc(C2CCOC2)cc(N2CC3(CC3)c3cnc(Cl)cc32)n1] |
| | SM: 3-bromotetrahydrofuran |
| | 53 mg, 48% yield, LCMS m/z = 393.2 [M + H]⁺. |

### Preparation 145

### 6′-chloro-1′-(2-(1,1-difluoroethyl)-6-(tetrahydrofuran-2-yl)pyrimidin-4-yl)-1′,2′-dihydrospiro[cyclopropane-1,3′-pyrrolo[3,2-c]pyridine]

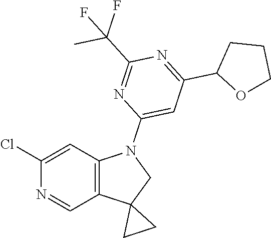 [SMILES: CC(F)(F)c1nc(C2CCCO2)cc(N2CC3(CC3)c3cnc(Cl)cc32)n1]

A mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 150 mg, 420 μmol), tetrahydrofuran-2-carboxylic acid (146 mg, 1.26 mmol), Cs₂CO₃ (410 mg, 1.26 mmol) in DMF (10 mL) was stirred under N₂. In a separate vial, NiCl₂ glyme (9.2 mg, 42 μmol), dtbbpy (16.9 mg, 63.0 μmol) and Ir[dF(CF₃)ppy]₂(dtbpy)PF₆ (4.71 mg, 4.20 μmol) were combined in DMF (10 mL) under N₂. The catalyst mixture was sonicated and added to the reaction vial. The reaction mixture was stirred and irradiated with blue LED (450 nm) at rt for 72 h under N₂. The mixture was diluted with EtOAc, washed with water (×5) and brine. The organic layer was separated, dried, and filtered. The filtrate was concentrated and the crude was purified by chromatography on silica gel (0-80% EtOAc in heptane) to give 6′-chloro-1′-(2-(1,1-difluoroethyl)-6-(tetrahydrofuran-2-yl)pyrimidin-4-yl)-1′,2′-dihydrospiro[cyclopropane-1,3′-pyrrolo[3,2-c]pyridine](26 mg, 16% yield) as a white powder. LCMS m/z=393.1 [M+H]⁺.

### Preparation 146

### 2-(6-(6′-chlorospiro[cyclopropane-1,3′-pyrrolo[3,2-c]pyridin]-1′(2′H)-yl)-2-(1,1-difluoroethyl)pyrimidin-4-yl)cyclopropane-1-carbonitrile

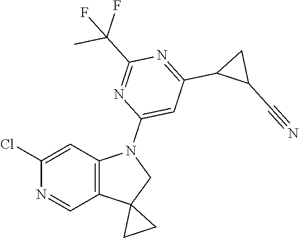 [SMILES: CC(F)(F)c1nc(C2CC2C#N)cc(N2CC3(CC3)c3cnc(Cl)cc32)n1]

To a solution of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 50 mg, 140 μmol) and 2-(4,4,5,5-tetramethyl-1,3,2-dioxaborolan-2-yl)cyclopropane-1-carbonitrile (32.4 mg, 168 μmol) in dioxane (2 mL) was added Cs₂CO₃ (2 M in water, 140 μL), Pd(OAc)₂ (1.6 mg, 7 μmol) and CataCXium® A (2.5 mg, 7 μmol). The reaction mixture was heated at 90° C. for 2 h. The mixture was diluted with EtOAc and washed with aq. NaHCO₃. The organic layer was dried and concentrated under vacuum. The crude was purified by chromatography on silica gel (0-90% EtOAc in heptane) to give 2-(6-(6′-chlorospiro[cyclopropane-1,3′-pyrrolo[3,2-c]pyridin]-1′(2′H)-yl)-2-(1,1-difluoroethyl)pyrimidin-4-yl)cyclopropane-1-carbonitrile (32 mg, 59% yield). LCMS m/z=388.1 [M+H]⁺.

### Preparation 147

### 6′-chloro-1′-(2-(1,1-difluoroethyl)-6-(methylsulfonyl)pyrimidin-4-yl)-1′,2′-dihydrospiro[cyclopropane-1,3′-pyrrolo[3,2-c]pyridine]

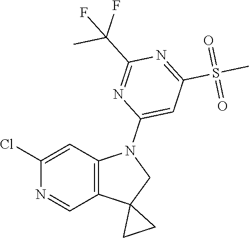 [SMILES: CC(F)(F)c1nc(N2CC3(CC3)c3cnc(Cl)cc32)cc(S(C)(=O)=O)n1]

To a solution of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 200 mg, 560 μmol) in THF (3 mL) at 0° C. was added an aqueous solution of sodium methanethiolate (2.14 M, 785 μL, 90% purity, 15% w/w). The reaction mixture was stirred from 0° C. to rt for 4 h. The mixture was quenched with water and extracted with EtOAc. The organic layer was separated, dried and concentrated under reduced pressure and used without further purification. LCMS m/z=369.1 [M+H]⁺.

The crude was dissolved in DCM (3 mL) and cooled to 0° C. MCPBA (251 mg, 1.12 mmol, 77% purity) was added. The reaction mixture was stirred at rt for 16 h and then treated with sat. aq. Na₂S₂O₃. The mixture was extracted with DCM, washed with sat. aq. NaHCO₃, water and brine, dried over Na₂SO₄ and concentrated under reduced pressure. The residue was purified by chromatography on silica gel (0-80% EtOAc in heptane) to give 6′-chloro-1′-(2-(1,1-difluoroethyl)-6-(methylsulfonyl)pyrimidin-4-yl)-1′,2′-dihydrospiro[cyclopropane-1,3′-pyrrolo[3,2-c]pyridine](32 mg, 14% yield) as a white powder. LCMS m/z=401.1 [M+H]⁺.

### Preparation 148

### 6-chloro-1-(2-(1,1-difluoroethyl)-6-methoxypyrimidin-4-yl)-2,3-dihydro-1H-pyrrolo[3,2-c]pyridine

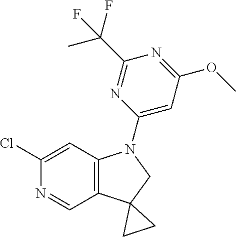 [SMILES: COc1cc(N2CC3(CC3)c3cnc(Cl)cc32)nc(C(C)(F)F)n1]

To a solution of 6-chloro-1-(6-chloro-2-(1,1-difluoroethyl)pyrimidin-4-yl)-2,3-dihydro-1H-pyrrolo[3,2-c]pyridine (Preparation 72, 50 mg, 151 μmol) in MeOH (2 mL) was added NaOMe (0.5 M, 302 μL) at rt. The reaction mixture was stirred at 60° C. for 1 h. The mixture was concentrated and purified by chromatography on silica gel (0-90% EtOAc in heptane) to give 6-chloro-1-(2-(1,1-difluoroethyl)-6-methoxypyrimidin-4-yl)-2,3-dihydro-1H-pyrrolo[3,2-c]pyridine (21 mg, 43% yield). LCMS m/z=327.0 [M+H]⁺.

### Preparation 149

### 6-(6-chloro-2,3-dihydro-1H-pyrrolo[3,2-c]pyridin-1-yl)-2-(1,1-difluoroethyl)-N-methylpyrimidin-4-amine

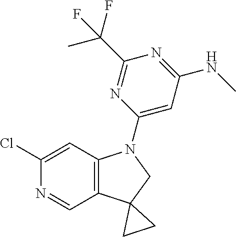 [SMILES: CNc1cc(N2CC3(CC3)c3cnc(Cl)cc32)nc(C(C)(F)F)n1]

A mixture of 6-chloro-1-(6-chloro-2-(1,1-difluoroethyl)pyrimidin-4-yl)-2,3-dihydro-1H-pyrrolo[3,2-c]pyridine (Preparation 72, 50 mg, 151 μmol) and methanamine (2 M in THF, 2 mL) was stirred at rt overnight. The mixture was concentrated under reduced pressure and purified by chromatography on silica gel (0-70% EtOAc-EtOH 3:1 with 2% NH₄OH in heptane) to give 6-(6-chloro-2,3-dihydro-1H-pyrrolo[3,2-c]pyridin-1-yl)-2-(1,1-difluoroethyl)-N-methylpyrimidin-4-amine (15 mg, 30% yield). LCMS m/z=326.1 [M+H]⁺.

### Preparation 150

### tert-butyl 6′-acetamido-1′-(4-(1,1-difluoroethyl)pyrimidin-2-yl)-1′,2′-dihydrospiro[piperidine-4,3′-pyrrolo[3,2-c]pyridine]-1-carboxylate

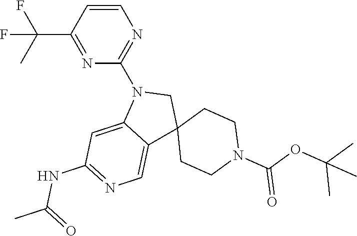 [SMILES: CC(=O)Nc1cc2c(cn1)C1(CCN(C(=O)OC(C)(C)C)CC1)CN2c1nccc(C(C)(F)F)n1]

tert-Butyl 6′-acetamido-1′-(4-(1,1-difluoroethyl)pyrimidin-2-yl)-1′,2′-dihydrospiro[piperidine-4,3′-pyrrolo[3,2-c]pyridine]-1-carboxylate was prepared using a similar method to the one described in Example 12, from tert-butyl 6′-chloro-1′-(4-(1,1-difluoroethyl)pyrimidin-2-yl)-1′,2′-dihydrospiro[piperidine-4,3′-pyrrolo[3,2-c]pyridine]-1-carboxylate (Preparation 68) and acetamide. LCMS m/z=489.2 [M+H]⁺.

### Preparation 151

### tert-butyl 6′-acetamido-1′-(2-(1,1-difluoroethyl)pyrimidin-4-yl)-1′,2′-dihydrospiro[piperidine-4,3′-pyrrolo[3,2-c]pyridine]-1-carboxylate

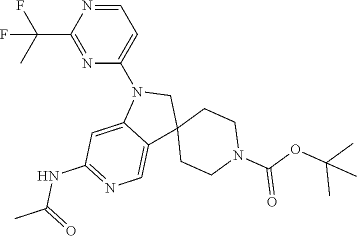 [SMILES: CC(=O)Nc1cc2c(cn1)C1(CCN(C(=O)OC(C)(C)C)CC1)CN2c1ccnc(C(C)(F)F)n1]

tert-Butyl 6′-acetamido-1′-(2-(1,1-difluoroethyl)pyrimidin-4-yl)-1′,2′-dihydrospiro[piperidine-4,3′-pyrrolo[3,2-c]pyridine]-1-carboxylate was prepared using a similar method to the one described in Example 12, from tert-butyl 6′-chloro-1′-(2-(1,1-difluoroethyl)pyrimidin-4-yl)- 1′,2′-dihydrospiro[piperidine-4,3′-pyrrolo[3,2-c]pyridine]-1-carboxylate (Preparation 69) and acetamide. LCMS m/z=489.2 [M+H]⁺.

### Preparation 152

### tert-butyl 6′-acetamido-1′-(6-(1,1-difluoroethyl)pyridin-2-yl)-1′,2′-dihydrospiro[piperidine-4,3′-pyrrolo[3,2-c]pyridine]-1-carboxylate

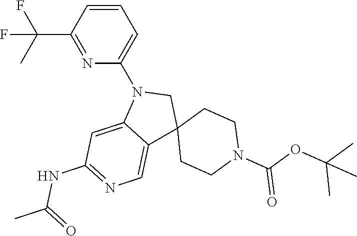 [SMILES: CC(=O)Nc1cc2c(cn1)C1(CCN(C(=O)OC(C)(C)C)CC1)CN2c1cccc(C(C)(F)F)n1]

tert-Butyl 6′-acetamido-1′-(6-(1,1-difluoroethyl)pyridin-2-yl)-1′,2′-dihydrospiro[piperidine-4,3′-pyrrolo[3,2-c]pyridine]-1-carboxylate was prepared using a similar method to the one described in Example 12, from tert-butyl 6′-chloro-1′-(6-(1,1-difluoroethyl)pyridin-2-yl)-1′,2′-dihydrospiro[piperidine-4,3′-pyrrolo[3,2-c]pyridine]-1-carboxylate (Preparation 70) and acetamide. LCMS m/z=488.2 [M+H]⁺.

### Preparations 153 and 154

### N-(1′-(2-chloro-6-methylpyrimidin-4-yl)-1′,2′-dihydrospiro[cyclopropane-1,3′-pyrrolo[3,2-c]pyridin]-6′-yl)acetamide and N-(1′-(4-chloro-6-methylpyrimidin-2-yl)-1′,2′-dihydrospiro[cyclopropane-1,3′-pyrrolo[3,2-c]pyridin]-6′-yl)acetamide

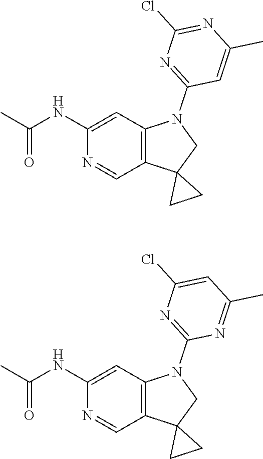

A mixture of N-(1′,2′-dihydrospiro[cyclopropane-1,3′-pyrrolo[3,2-c]pyridin]-6′-yl)acetamide (Preparation 7, 2 g, 9.84 mmol), NaOtBu (2.84 g, 29.52 mmol) and Xantphos Pd G3 (491.17 mg, 492 μmol) in dioxane (100 mL) was purged with N₂. 2,4-Dichloro-6-methyl-pyrimidine (3.21 g, 19.68 mmol) was added and the reaction was stirred at 90° C. for 1 h. The mixture was quenched with 1M NaOH, diluted with brine and extracted with EtOAc. The solid formed in the organic layer was separated by filtration and washed with EtOAc to give 1.32 g solid of product 1, Preparation 153. The filtrate was washed with water, dried and concentrated. The crude was purified by chromatography on silica gel (0-80% EtOAc-EtOH 3:1 with 2% NH₄OH in heptane) to give another 710 mg white powder of N-(1′-(2-chloro-6-methylpyrimidin-4-yl)-1′,2′-dihydrospiro[cyclopropane-1,3′-pyrrolo[3,2-c]pyridin]-6′-yl)acetamide and 71 mg of N-(1′-(4-chloro-6-methylpyrimidin-2-yl)-1′,2′-dihydrospiro[cyclopropane-1,3′-pyrrolo[3,2-c]pyridin]-6′-yl)acetamide. The regio-isomers were confirmed by 2D-NMR.

Preparation 153: N-(1′-(2-chloro-6-methylpyrimidin-4-yl)-1′,2′-dihydrospiro[cyclopropane-1,3′-pyrrolo[3,2-c]pyridin]-6′-yl)acetamide (2.03 g, 62% yield). LCMS m/z=330.0 [M+H]⁺ ¹H NMR (DMSO-d₆, 400 MHz) δ 10.30 (s, 1H), 8.94 (s, 1H), 7.70 (s, 1H), 6.74 (s, 1H), 4.12 (s, 2H), 2.39 (s, 3H), 2.07 (s, 3H), 1.20-1.10 (m, 4H).

Preparation 154: N-(1′-(4-chloro-6-methylpyrimidin-2-yl)-1′,2′-dihydrospiro[cyclopropane-1,3′-pyrrolo[3,2-c]pyridin]-6′-yl)acetamide. (71 mg, 2% yield). LCMS m/z=330.0 [M+H]⁺ ¹H NMR (DMSO-d₆, 400 MHz) δ 10.27 (s, 1H), 9.05 (br s, 1H), 7.65 (s, 1H), 7.09 (s, 1H), 4.19 (s, 2H), 2.45 (s, 3H), 2.07 (s, 3H), 1.20-1.10 (m, 4H)

### Preparation 155

### N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide

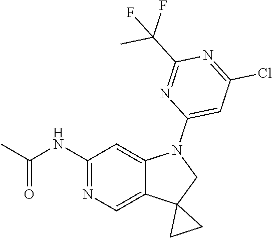 [SMILES: CC(=O)Nc1cc2c(cn1)C1(CC1)CN2c1cc(Cl)nc(C(C)(F)F)n1]

To a solution of N-(1′,2′-dihydrospiro[cyclopropane-1,3′-pyrrolo[3,2-c]pyridin]-6′-yl)acetamide (Preparation 7, 5 g, 24.60 mmol), 4,6-dichloro-2-(1,1-difluoroethyl)pyrimidine (Preparation 30, 6.55 g, 30.75 mmol) and K₃PO₄ (10.44 g, 49.20 mmol) in DMF (50 mL) was stirred at 70° C. overnight. The cooled mixture was diluted with DMF (10 mL) then water (100 mL) and the resulting solid was filtered off and dried to give N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide as a tan solid, 5.9 g. The aqueous layer was extracted with EtOAc, the combined organic extracts filtered through silica gel and concentrated in vacuo to provide further title compound. LCMS m/z=380 [M+H]⁺;

### Preparation 156

### N-(1′-(2-(1,1-difluoroethyl)-6-((2,4-dimethoxybenzyl)amino)pyrimidin-4-yl)-1′,2′-dihydrospiro[cyclopropane-1,3′-pyrrolo[3,2-c]pyridin]-6′-yl)acetamide

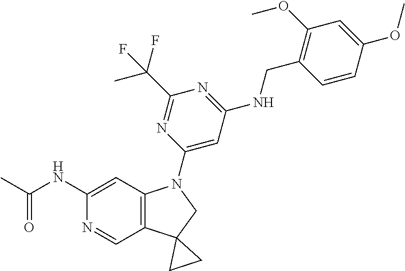 [SMILES: COc1ccc(CNc2cc(N3CC4(CC4)c4cnc(NC(C)=O)cc43)nc(C(C)(F)F)n2)c(OC)c1]

To a vial was added N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 150 mg, 395 μmol), (2,4-dimethoxyphenyl)methanamine (79.25 mg, 474 μmol), K₂CO₃ (163.75 mg, 1.18 mmol) and DMF (3 mL) and the reaction mixture was stirred at 60° C. overnight. The mixture was diluted with EtOAc and washed with aq. NaHCO₃, H₂O and brine. The organic layer was dried over Na₂SO₄ and the solvent removed in vacuo. The residue was purified by column chromatography (0 to 100% EtOAc: heptane) to provide N-(1′-(2-(1,1-difluoroethyl)-6-((2,4-dimethoxybenzyl)amino)pyrimidin-4-yl)-1′,2′-dihydrospiro[cyclopropane-1,3′-pyrrolo[3,2-c]pyridin]-6′-yl)acetamide as a yellow oil, 253 mg. LCMS m/z=511 [M+H]⁺

### Preparation 157

### N-(1′-(6-bromo-4-methylpyridin-2-yl)-1′,2′-dihydrospiro[cyclopropane-1,3′-pyrrolo[3,2-c]pyridin]-6′-yl)acetamide

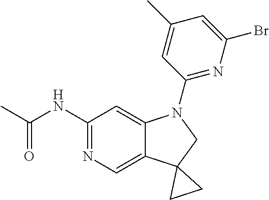 [SMILES: CC(=O)Nc1cc2c(cn1)C1(CC1)CN2c1cc(C)cc(Br)n1]

A solution of N-(1′,2′-dihydrospiro[cyclopropane-1,3′-pyrrolo[3,2-c]pyridin]-6′-yl)acetamide (Preparation 7, 5 g, 24.6 mmol) in THF (250 mL) was purged with N₂, KHMDS (1 M, 54 mL) added and the solution stirred for 20 min. 2,6-Dibromo-4-methyl-pyridine (6.2 g, 24.6 mmol) was added and the reaction was stirred at rt overnight. The mixture was quenched with 1M NaOH, diluted with brine and extracted with EtOAc. The organic layer was separated, dried and concentrated. The crude was purified by chromatography on silica gel (0-80% EtOAc-EtOH 3:1 with 2% NH₄OH in heptane) to give N-(1′-(6-bromo-4-methylpyridin-2-yl)-1′,2′-dihydrospiro[cyclopropane-1,3′-pyrrolo[3,2-c]pyridin]-6′-yl)acetamide (2.8 g, 30% yield) as a white powder. LCMS m/z=373.0, 375.0 [M+H]⁺.

### Preparation 158

### Ethyl 2-(4-chloro-6-methylpyrimidin-2-yl)-2,2-difluoroacetate

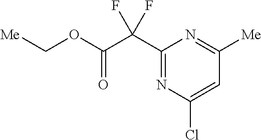 [SMILES: CCOC(=O)C(F)(F)c1nc(C)cc(Cl)n1]

A mixture of ethyl 2-bromo-2,2-difluoro-acetate (978 mg, 4.82 mmol) and copper (613 mg, 9.64 mmol) in DMSO (8 mL) was purged with N₂ and stirred at rt for 1 h. To this was added a solution of 2-bromo-4-chloro-6-methyl-pyrimidine (500 mg, 2.41 mmol) in DMSO (2 mL) and the resulting mixture stirred at 40° C. overnight. The reaction was diluted with water and extracted with EtOAc (×2). The combined organics were dried and concentrated and the residue purified by chromatography on silica gel (0-40% EtOAc/heptane) to give ethyl 2-(4-chloro-6-methylpyrimidin-2-yl)-2,2-difluoroacetate as a colourless oil (440 mg, 72%): LCMS m/z=251 [M+H]⁺.

### Preparation 159

### Ethyl 2-(6-chloro-4-methylpyridin-2-yl)-2,2-difluoroacetate

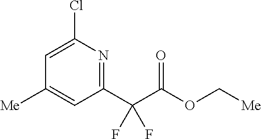 [SMILES: CCOC(=O)C(F)(F)c1cc(C)cc(Cl)n1]

The title compound was prepared as a yellow oil (585 mg, 63%) from ethyl 2-bromo-2,2-difluoro-acetate and 2-bromo-6-chloro-4-methylpyridine using an analogous method to that described for Preparation 158. LCMS m/z=250 [M+H]⁺.

### Preparation 160

### 2-(6-chloro-4-methylpyridin-2-yl)-2,2-difluoroethan-1-ol

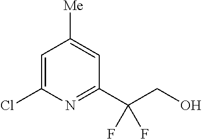 [SMILES: Cc1cc(Cl)nc(C(F)(F)CO)c1]

To a solution of ethyl 2-(6-chloro-4-methylpyridin-2-yl)-2,2-difluoroacetate (Preparation 159, 500 mg, 2.0 mmol) in EtOH (9 mL) was added NaBH₄ (152 mg, 4.0 mmol) and the mixture was stirred at rt for 1 h. The reaction was neutralized with 2M HCl and evaporated to remove the solvent and the residue purified by chromatography on silica gel (0-80% [(3:1 EtOAc/EtOH)+2% NH₄OH]/heptane) to give 2-(6-chloro-4-methylpyridin-2-yl)-2,2-difluoroethan-1-ol as a white solid (305 mg, 73%). LCMS m/z=208 [M+H]⁺.

### Preparation 161

### 2-chloro-6-(1,1-difluoro-2-methoxyethyl)-4-methylpyridine

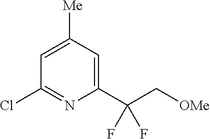 [SMILES: COCC(F)(F)c1cc(C)cc(Cl)n1]

To a mixture of 2-(6-chloro-4-methylpyridin-2-yl)-2,2-difluoroethan-1-ol (Preparation 160, 140 mg, 0.674 mmol) in DMF (3 mL) at 0° C. was added NaH (54 mg, 1.35 mmol, 60% in mineral oil) and stirred for 5 min, followed by the addition of MeI (957 mg, 6.74 mmol) and the reaction mixture stirred at rt for 1 h. The reaction mixture was diluted with EtOAc, washed with water (3×) and brine. The organic layer was separated, dried and evaporated to dryness and the residue purified by chromatography on silica gel (0-100% EtOAc/heptane) to give 2-chloro-6-(1,1-difluoro-2-methoxyethyl)-4-methylpyridine (91 mg, 61%). LCMS m/z=222 [M+H]⁺.

### Preparation 162

### 2-chloro-4-methyl-6-(1,1,2-trifluoroethyl)pyridine

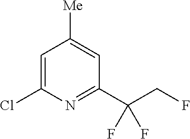 [SMILES: Cc1cc(Cl)nc(C(F)(F)CF)c1]

To a solution of 2-(6-chloro-4-methylpyridin-2-yl)-2,2-difluoroethan-1-ol (Preparation 160, 150 mg, 0.72 mmol) in DCM (4 mL) at 0° C. was added DAST (191 mL, 1.45 mmol) and the resulting mixture stirred at 60° C. for 3 h. The reaction was cooled to 0° C., quenched with MeOH, solid NaHCO₃ added slowly, the mixture diluted with water and extracted with DCM. The organic layer was separated, dried and evaporated to dryness to give 2-chloro-4-methyl-6-(1,1,2-trifluoroethyl)pyridine (94 mg) which was used without further purification. LCMS m/z=210 [M+H]⁺

### Preparation 163

### 2-(1,1-difluoropropyl)pyrimidine-4,6-diol

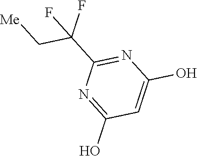 [SMILES: CCC(F)(F)c1nc(O)cc(O)n1]

To a solution of malonamide (210 mg, 2.06 mmol) in EtOH (5 mL) was added sodium tert-butoxide (692 mg, 7.20 mmol) and the mixture stirred at 25° C. for 30 min. To this was added ethyl 2,2-difluorobutanoate (642 mg, 4.22 mmol) and the reaction was heated under reflux for 24 h. The reaction mixture was cooled to rt, HCl (4 N, 2.57 mL) was added and the precipitate removed by filtration. The filtrate was concentrated under reduced pressure and the residue partitioned between EtOAc and H₂O (minimum) and separated. The combined organics were dried (MgSO₄) and evaporated to dryness. The residue was purified by silica gel chromatography (0-100% EtOAc/EtOH (3:1) in heptanes) to give 2-(1,1-difluoropropyl)pyrimidine-4,6-diol as a white solid (104 mg, 27%). LCMS m/z=191 [M+H]⁺.

### Preparation 164

### 4,6-dichloro-2-(1,1-difluoropropyl)pyrimidine

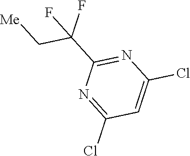 [SMILES: CCC(F)(F)c1nc(Cl)cc(Cl)n1]

The title compound was prepared as a colorless oil (200 mg, 62%) from 2-(1,1-difluoropropyl)pyrimidine-4,6-diol (Preparation 163) using an analogous method as described for Preparation 30. ¹H NMR (500 MHz, CDCl₃) δ ppm 7.50 (s, 1H), 2.29-2.42 (m, 2H), 1.08 (t, J=7.48 Hz, 3H).

### Preparation 165

### 1,3-dioxoisoindolin-2-yl 1-methyl-2-oxabicyclo[2.1.1]hexane-4-carboxylate

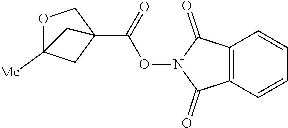 [SMILES: CC12CC(C(=O)ON3C(=O)c4ccccc4C3=O)(CO1)C2]

To a mixture of 1-methyl-2-oxabicyclo[2.1.1]hexane-4-carboxylic acid (145 mg, 1.02 mmol), DMAP (6.23 mg, 0.051 mmol) and 2-hydroxyisoindoline-1,3-dione (166.4 mg, 1.02 mmol) in DCM (5 mL) was added a solution of DCC (253 mg, 1.22 mmol) in DCM (2 mL) and the mixture stirred at 40° C. overnight. The solids were removed by filtration and the filtrate concentrated to give 1,3-dioxoisoindolin-2-yl 1-methyl-2-oxabicyclo[2.1.1]hexane-4-carboxylate as an off-white powder (303 mg, crude) which was used without further purification. ¹H NMR (500 MHz, DMSO-d₆) δ 8.00-7.90 (m, 4H), 3.98 (s, 2H), 2.26 (br d, 2H, J=4.3 Hz), 1.93 (br d, 2H, J=4.1 Hz), 1.42 (s, 3H).

### Preparation 166

### 1,3-dioxoisoindolin-2-yl 1-fluorocyclopropane-1-carboxylate

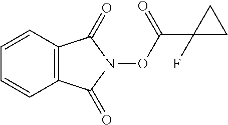 [SMILES: O=C1c2ccccc2C(=O)N1OC(=O)C1(F)CC1]

N,N′-Diisopropylmethanediimine (133.4 mg, 1.06 mmol) was added to a solution of 1-fluorocyclopropanecarboxylic acid (100 mg, 0.961 mmol) and 2-hydroxyisoindoline-1,3-dione (196 mg, 1.20 mmol) in THF (3 mL) and the mixture stirred at rt for 1.5 h. The reaction mixture was evaporated to dryness under reduced pressure and the residue purified by column chromatography (heptane) to afford 1,3-dioxoisoindolin-2-yl 1-fluorocyclopropane-1-carboxylate (183 mg, 79%)

### Preparation 167

### 6′-chloro-1′-(6-methyl-2-(oxetan-3-yl)pyrimidin-4-yl)-1′,2′-dihydrospiro[cyclopropane-1,3′-pyrrolo[3,2-c]pyridine]

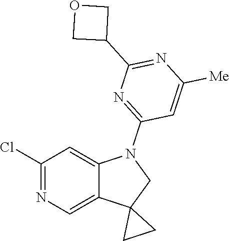 [SMILES: Cc1cc(N2CC3(CC3)c3cnc(Cl)cc32)nc(C2COC2)n1]

A solution of 6′-chloro-1′-(6-methylpyrimidin-4-yl)-1′,2′-dihydrospiro[cyclopropane-1,3′-pyrrolo[3,2-c]pyridine] (Preparation 101, 200 mg, 0.733 mmol), conc. H₂SO₄ (144 mg, 1.47 mmol), 3-iodooxetane (270 mg, 1.47 mmol) and ferrous sulfate heptahydrate (61 mg, 0.22 mmol) in DMSO (7 mL) was heated at 60° C. Hydrogen peroxide (74.8 mg, 2.20 mmol) was added dropwise and after 2 min another portion of ferrous sulfate heptahydrate and hydrogen peroxide (74.8 mg, 2.20 mmol) was added and the reaction mixture stirred at 60° C. for 1 h. The cooled mixture was poured into 0.2M NaOH (saturated with NaCl) and extracted with EtOAc (2×). The combined organics were dried and concentrated and the residue purified by chromatography on silica gel (0-80% [(3:1 EtOAc/EtOH)+2% NH₄OH]/heptane) to give 6′-chloro-1′-(6-methyl-2-(oxetan-3-yl)pyrimidin-4-yl)-1′,2′-dihydrospiro[cyclopropane-1,3′-pyrrolo[3,2-c]pyridine](24 mg, 10%). LCMS m/z=329 [M+H]⁺.

### Preparation 168

### 1′-(2-bromopyrimidin-4-yl)-6′-chloro-1′,2′-dihydrospiro[cyclopropane-1,3′-pyrrolo[3,2-c]pyridine]

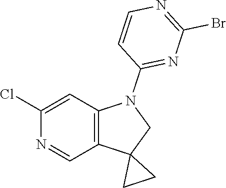 [SMILES: Clc1cc2c(cn1)C1(CC1)CN2c1ccnc(Br)n1]

To a solution of 6′-chloro-1′,2′-dihydrospiro[cyclopropane-1,3′-pyrrolo[3,2-c]pyridine] (Preparation 4, 600 mg, 3.32 mmol) in THF (24 mL) was added potassium tert-butoxide (2 M in THF, 3.32 mL) and the reaction mixture stirred at rt for 30 min. The reaction mixture was diluted with EtOAc and washed with brine. The combined organics were dried, concentrated and the residue purified by chromatography on silica gel (0-80% (3:1 EtOAc/EtOH+2% NH₄OH) in heptane) to give 1′-(2-bromopyrimidin-4-yl)-6′-chloro-1′,2′ -dihydrospiro[cyclopropane-1,3′-pyrrolo[3,2-c]pyridine](121 mg, 11%). LCMS m/z=337/339 [M+H]⁺.

### Preparation 169

### Ethyl 2-(4-(6′-chlorospiro[cyclopropane-1,3′-pyrrolo[3,2-c]pyridin]-1′(2′H)-yl)pyrimidin-2-yl)-2,2-difluoroacetate

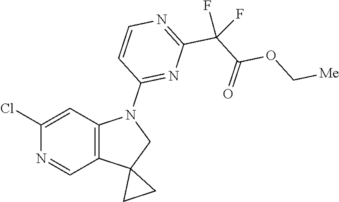 [SMILES: CCOC(=O)C(F)(F)c1nccc(N2CC3(CC3)c3cnc(Cl)cc32)n1]

A mixture of ethyl 2-bromo-2,2-difluoroacetate (144.3 mg, 0.711 mmol) and copper (90.4 mg, 1.42 mmol) in DMSO (2 mL) was stirred at 40° C. for 30 min. To this was added a solution of 1′-(2-bromopyrimidin-4-yl)-6′-chloro-1′,2′-dihydrospiro[cyclopropane-1,3′-pyrrolo[3,2-c]pyridine] (Preparation 168, 120 mg, 0.355 mmol) in DMSO (2 mL) and the mixture stirred at 40° C. overnight. The reaction mixture was diluted with EtOAc, washed with water (3×) and brine. The combined organics were dried and evaporated to dryness and the residue purified by chromatography on silica gel (0-100% EtOAc/heptane) to give ethyl 2-(4-(6′-chlorospiro[cyclopropane-1,3′-pyrrolo[3,2-c]pyridin]-1′(2′H)-yl)pyrimidin-2-yl)-2,2-difluoroacetate as a white powder (94 mg, 69%). LCMS m/z=381 [M+H]⁺.

### Preparation 170

### 2-(4-(6′-chlorospiro[cyclopropane-1,3′-pyrrolo[3,2-c]pyridin]-1′(2′H)-yl)pyrimidin-2-yl)-2,2-difluoroethan-1-ol

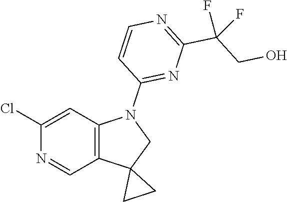 [SMILES: OCC(F)(F)c1nccc(N2CC3(CC3)c3cnc(Cl)cc32)n1]

NaBH₄ (36 mg, 0.945 mmol) was added to a solution of ethyl 2-(4-(6′-chlorospiro[cyclopropane-1,3′-pyrrolo[3,2-c]pyridin]-1′(2′H)-yl)pyrimidin-2-yl)-2,2-difluoroacetate (Preparation 169, 90 mg, 0.236 mmol) in EtOH (5 mL) and the mixture stirred at rt for 1 h. The reaction was quenched with 2M HCl and concentrated to remove the volatiles. The residue was diluted with NaHCO₃/brine and extracted with EtOAc (3×). The combined organics were dried and concentrated to give 2-(4-(6′-chlorospiro[cyclopropane-1,3′-pyrrolo[3,2-c]pyridin]-1′(2′H)-yl)pyrimidin-2-yl)-2,2-difluoroethan-1-ol as a white powder (78 mg, crude) which was used without further purification. LCMS m/z=339 [M+H]⁺.

### Preparation 171

### 6′-chloro-1′-(2-(1,1-difluoro-2-methoxyethyl)pyrimidin-4-yl)-1′,2′-dihydrospiro[cyclopropane-1,3′-pyrrolo[3,2-c]pyridine]

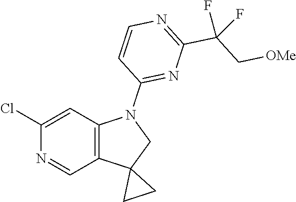 [SMILES: COCC(F)(F)c1nccc(N2CC3(CC3)c3cnc(Cl)cc32)n1]

NaH (17.71 mg, 0.443 mmol, 60% in mineral oil) was added to a mixture of 2-(4-(6′-chlorospiro[cyclopropane-1,3′-pyrrolo[3,2-c]pyridin]-1′(2′H)-yl)pyrimidin-2-yl)-2,2-difluoroethan-1-ol (Preparation 170, 75 mg, 0.221 mmol) and MeI (314 mg, 2.21 mmol) in THF (3 mL) and the mixture stirred at rt overnight. The solids were removed by filtration and the filtrate evaporated to dryness. The residue was purified by chromatography on silica gel (0-100% EtOAc/heptane) to give 6′-chloro-1′-(2-(1,1-difluoro-2-methoxyethyl)pyrimidin-4-yl)-1′,2′-dihydrospiro[cyclopropane-1,3′-pyrrolo[3,2-c]pyridine] as a white solid (46 mg, 59%). LCMS m/z=353 [M+H]⁺.

### Preparation 172

### 6′-chloro-1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridine]

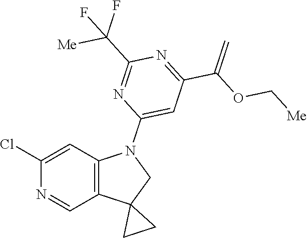 [SMILES: C=C(OCC)c1cc(N2CC3(CC3)c3cnc(Cl)cc32)nc(C(C)(F)F)n1]

To a mixture of 6′-chloro-1′-(6-chloro-2-(1,1-difluoroethyl)pyrimidin-4-yl)-1′,2′-dihydrospiro[cyclopropane-1,3′-pyrrolo[3,2-c]pyridine] (Preparation 71, 2 g, 5.60 mmol) and Pd(PPh₃)₄Cl₂ (393 mg, 0.56 mmol) in DMF (20 mL) was added tributyl(1-ethoxyvinyl)stannane (2.22 g, 6.16 mmol) at rt. The reaction mixture was purged with N₂, then heated at 70° C. for 2 h. After cooling, the reaction was quenched with saturated aq. KF solution and diluted with EtOAc. The precipitate was filtered off and the filtrate was collected and washed with water (3×) and brine. The organic layer was dried and concentrated. The white solid diluted with EtOAc, filtered, rinsed with heptane and dried to give 6′-chloro-1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridine](1.9 g, 77% yield). LCMS m/z=393 [M+H]⁺.

### Preparation 173

### ethyl 6-(6′-chlorospiro[cyclopropane-1,3′-pyrrolo[3,2-c]pyridin]-1′(2′H)-yl)-2-(1,1-difluoroethyl)pyrimidine-4-carboxylate

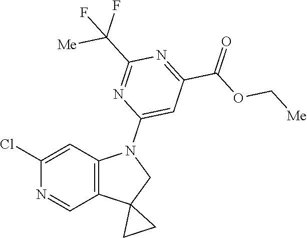 [SMILES: CCOC(=O)c1cc(N2CC3(CC3)c3cnc(Cl)cc32)nc(C(C)(F)F)n1]

A mixture of 6′-chloro-1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridine] (Preparation 172, 600 mg, 1.53 mmol), NaIO₄ (1.31 g, 6.11 mmol), KMnO₄ (96 mg, 0.611 mmol) and water (8 mL) was stirred at rt overnight. The reaction mixture was extracted with EtOAc, the organic layer was separated, washed with water, brine, then dried, and concentrated. The crude was purified by chromatography on silica gel (0-60% EtOAc in heptane) to give ethyl 6-(6′-chlorospiro[cyclopropane-1,3′-pyrrolo[3,2-c]pyridin]-1′(2′H)-yl)-2-(1,1-difluoroethyl)pyrimidine-4-carboxylate (122 mg, 20% yield). LCMS m/z=395 [M+H]⁺.

### Preparation 174

### (6-(6′-chlorospiro[cyclopropane-1,3′-pyrrolo[3,2-c]pyridin]-1′(2′H)-yl)-2-(1,1-difluoroethyl)pyrimidin-4-yl)methanol

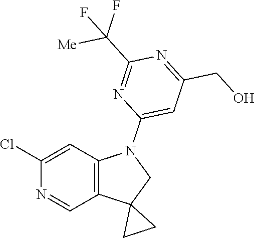 [SMILES: CC(F)(F)c1nc(CO)cc(N2CC3(CC3)c3cnc(Cl)cc32)n1]

To a solution of ethyl 6-(6′-chlorospiro[cyclopropane-1,3′-pyrrolo[3,2-c]pyridin]-1′(2′H)-yl)-2-(1,1-difluoroethyl)pyrimidine-4-carboxylate (Preparation 173, 120 mg, 0.304 mmol) in EtOH (3 mL) was added NaBH₄ (35 mg, 0.912 mmol) and the reaction mixture was stirred at rt for 1 h. The solvent was removed in vacuo, the residue was diluted with EtOAc and washed with brine. The organic layer was separated, dried and concentrated to give (6-(6′-chlorospiro[cyclopropane-1,3′-pyrrolo[3,2-c]pyridin]-1′(2′H)-yl)-2-(1,1-difluoroethyl)pyrimidin-4-yl)methanol (106 mg, 98% yield) which was used in the next step without further purification. LCMS m/z=353 [M+H]⁺.

### Preparation 175

### 6′-chloro-1′-(2-(1,1-difluoroethyl)-6-(methoxymethyl)pyrimidin-4-yl)-1′,2′-dihydrospiro[cyclopropane-1,3′-pyrrolo[3,2-c]pyridine]

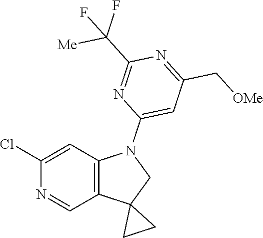 [SMILES: COCc1cc(N2CC3(CC3)c3cnc(Cl)cc32)nc(C(C)(F)F)n1]

To a solution of (6-(6′-chlorospiro[cyclopropane-1,3′-pyrrolo[3,2-c]pyridin]-1′(2′H)-yl)-2-(1,1-difluoroethyl)pyrimidin-4-yl)methanol (Preparation 174, 90 mg, 0.255 mmol) in DMF (2 mL) was added Cs₂CO₃ (166 mg, 0.510 mmol) and MeI (217 mg, 1.53 mmol) and the mixture stirred at rt overnight. The reaction mixture was diluted with EtOAc and washed with water (3×) and brine. The combined organics were dried and evaporated to dryness and the residue purified by chromatography on silica gel (0-60% EtOAc/heptane) to give 6′-chloro-1′-(2-(1,1-difluoroethyl)-6-(methoxymethyl)pyrimidin-4-yl)-1′,2′-dihydrospiro[cyclopropane-1,3′-pyrrolo[3,2-c]pyridine](15 mg, 16%). LCMS m/z=367 [M+H]⁺.

### Preparation 176

### 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-one

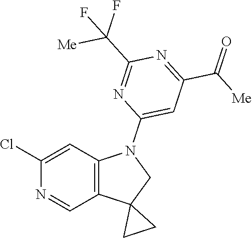 [SMILES: CC(=O)c1cc(N2CC3(CC3)c3cnc(Cl)cc32)nc(C(C)(F)F)n1]

To a solution of 6′-chloro-1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridine] (Preparation 172, 500 mg, 1.27 mmol) in acetone (8 mL) was added aq.HCl (2M, 1.91 mL) and the reaction mixture was stirred at rt for 2 h. The mixture was concentrated in vacuo and the residue was diluted with ether, the solid formed was filtered, rinsed with ether, then dried under vacuum to give 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-one (304 mg, 65% yield). LCMS m/z=365 [M+H]⁺.

### Preparation 177

### 2-(6-(6′-chlorospiro[cyclopropane-1,3′-pyrrolo[3,2-c]pyridin]-1′(2′H)-yl)-2-(1,1-difluoroethyl)pyrimidin-4-yl)propan-2-ol

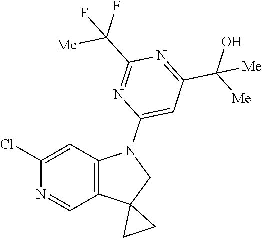 [SMILES: CC(C)(O)c1cc(N2CC3(CC3)c3cnc(Cl)cc32)nc(C(C)(F)F)n1]

A solution of 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-one (Preparation 176, 200 mg, 0.548 mmol) in THF (6 mL) was cooled to 0° C., methyl magnesium chloride (3M in THF, 0.2 mL) was added dropwise and the resulting mixture stirred from 0° C. to rt over 1 h. The reaction was quenched with water and extracted with EtOAc. The combined organics were washed with brine, dried and evaporated to dryness to give 2-(6-(6′-chlorospiro[cyclopropane-1,3′ -pyrrolo[3,2-c]pyridin]-1′(2′H)-yl)-2-(1,1-difluoroethyl)pyrimidin-4-yl)propan-2-ol (208 mg, crude) which was used without further purification. LCMS m/z=381 [M+H]⁺.

### Preparation 178

### 6′-chloro-1′-(2-(1,1-difluoroethyl)-6-(2-fluoropropan-2-yl)pyrimidin-4-yl)-1′,2′-dihydrospiro[cyclopropane-1,3′-pyrrolo[3,2-c]pyridine]

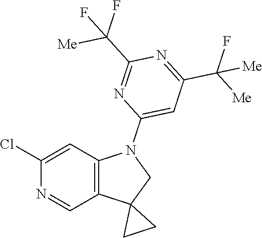 [SMILES: CC(C)(F)c1cc(N2CC3(CC3)c3cnc(Cl)cc32)nc(C(C)(F)F)n1]

A solution of 2-(6-(6′-chlorospiro[cyclopropane-1,3′-pyrrolo[3,2-c]pyridin]-1′(2′H)-yl)-2-(1,1-difluoroethyl)pyrimidin-4-yl)propan-2-ol (Preparation 177, 100 mg, 0.263 mmol) in DCM (3 mL) was cooled to −78° C., DAST (64 mg, 0.394 mmol) was added under N₂ followed by N,N-triethylamine trihydrofluoride (42 mg, 0.263 mmol) and the reaction mixture stirred 0° C. for 1 h. The reaction was quenched with aq. NaHCO₃ and the mixture extracted with EtOAc. The combined organics were dried and evaporated to dryness. The residue was purified by chromatography on silica gel (0-80% EtOAc/heptane) to give 6′-chloro-1′-(2-(1,1-difluoroethyl)-6-(2-fluoropropan-2-yl)pyrimidin-4-yl)-1′,2′-dihydrospiro[cyclopropane-1,3′-pyrrolo[3,2-c]pyridine](67 mg, 66%). LCMS m/z=383 [M+H]⁺.

### Preparation 179

### 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-ol

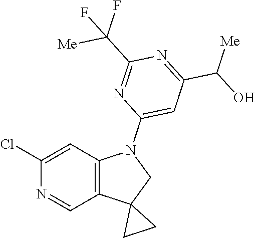 [SMILES: CC(O)c1cc(N2CC3(CC3)c3cnc(Cl)cc32)nc(C(C)(F)F)n1]

1-(6-(6′-Chlorospiro[cyclopropane-1,3′-pyrrolo[3,2-c]pyridin]-1′(2′H)-yl)-2-(1,1-difluoroethyl)pyrimidin-4-yl)ethan-1-ol was prepared (226 mg, 82%) from 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-one (Preparation 176) using an analogous method as described for Preparation 174. LCMS m/z=367 [M+H]⁺.

### Preparation 180

### 6′-chloro-1′-(2-(1,1-difluoroethyl)-6-(1-fluoroethyl)pyrimidin-4-yl)-1′,2′-dihydrospiro[cyclopropane-1,3′-pyrrolo[3,2-c]pyridine]

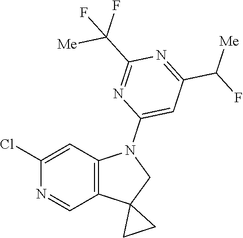 [SMILES: CC(F)c1cc(N2CC3(CC3)c3cnc(Cl)cc32)nc(C(C)(F)F)n1]

6′-Chloro-1′-(2-(1,1-difluoroethyl)-6-(1-fluoroethyl)pyrimidin-4-yl)-1′,2′-dihydrospiro[cyclopropane-1,3′-pyrrolo[3,2-c]pyridine] was prepared (73 mg, 60%) from 1-(6-(6′-chlorospiro[cyclopropane-1,3′-pyrrolo[3,2-c]pyridin]-1′(2′H)-yl)-2-(1,1-difluoroethyl)pyrimidin-4-yl)ethan-1-ol (Preparation 179) using an analogous method as described for Preparation 178. LCMS m/z=369 [M+H]⁺.

### Preparation 181

### Ethyl 2-(4-(6′-chlorospiro[cyclopropane-1,3′-pyrrolo[3,2-c]pyridin]-1′(2′H)-yl)-6-methylpyrimidin-2-yl)-2,2-difluoroacetate

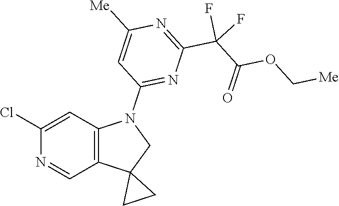 [SMILES: CCOC(=O)C(F)(F)c1nc(C)cc(N2CC3(CC3)c3cnc(Cl)cc32)n1]

A mixture of 6′-chloro-1′,2′-dihydrospiro[cyclopropane-1,3′-pyrrolo[3,2-c]pyridine](Preparation 4, 450 mg, 2.49 mmol), ethyl 2-(4-chloro-6-methylpyrimidin-2-yl)-2,2-difluoroacetate (Preparation 158, 892 mg, 2.49 mmol) and Cs₂CO₃ (1.62 g, 4.98 mmol) in DMF (10 mL) was heated at 40° C. overnight. The reaction was partitioned between water and EtOAc and the organic layer was separated. The combined organics were washed with H₂O (3×) then brine, dried and evaporated to dryness. The residue was purified by chromatography on silica gel (0-80% (3:1 EtOAc/EtOH (+2% NH₄OH) in heptane) to give ethyl 2-(4-(6′-chlorospiro[cyclopropane-1,3′-pyrrolo[3,2-c]pyridin]-1′(2′H)-yl)-6-methylpyrimidin-2-yl)-2,2-difluoroacetate as a white solid (303 mg, 31%). LCMS m/z=395.1 [M+H]⁺.

### Preparation 182

### 2-(4-(6′-chlorospiro[cyclopropane-1,3′-pyrrolo[3,2-c]pyridin]-1′(2′H)-yl)-6-methylpyrimidin-2-yl)-2,2-difluoroethan-1-ol

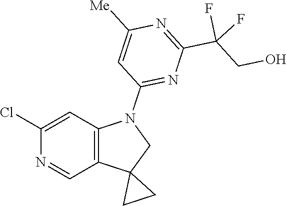 [SMILES: Cc1cc(N2CC3(CC3)c3cnc(Cl)cc32)nc(C(F)(F)CO)n1]

NaBH₄ (53.7 mg, 1.42 mmol) was added to a solution of ethyl 2-(4-(6′-chlorospiro[cyclopropane-1,3′-pyrrolo[3,2-c]pyridin]-1′(2′H)-yl)-6-methylpyrimidin-2-yl)-2,2-difluoroacetate (Preparation 181, 140 mg, 0.355 mmol) in EtOH (3 mL) and the mixture stirred at rt for 1 h. The reaction mixture was quenched with 2M HCl and concentrated to remove the volatiles. The residue was diluted with sat. aq. NaHCO₃, brine and extracted with EtOAc (2×). The combined organics were dried and evaporated to dryness to give 2-(4-(6′-chlorospiro[cyclopropane-1,3′-pyrrolo[3,2-c]pyridin]-1′(2′H)-yl)-6-methylpyrimidin-2-yl)-2,2-difluoroethan-1-ol as a white solid (66 mg, 53%). LCMS m/z=353.0 [M+H]⁺

### Preparation 183

### 6′-chloro-1′-(2-(1,1-difluoro-2-methoxyethyl)-6-methylpyrimidin-4-yl)-1′,2′-dihydrospiro[cyclopropane-1,3′-pyrrolo[3,2-c]pyridine]

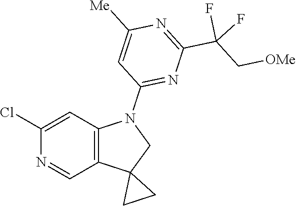 [SMILES: COCC(F)(F)c1nc(C)cc(N2CC3(CC3)c3cnc(Cl)cc32)n1]

To a mixture of 2-(4-(6′-chlorospiro[cyclopropane-1,3′-pyrrolo[3,2-c]pyridin]-1′(2′H)-yl)-6-methylpyrimidin-2-yl)-2,2-difluoroethan-1-ol (Preparation 182, 33 mg, 0.094 mmol) and MeI (58 μL, 0.940 mmol) in THF (1.5 mL) was added NaH (7 mg, 0.20 mmol, 60% purity) and the reaction stirred at rt for 24 h before additional NaH (7 mg, 0.20 mmol, 60% purity) was added. After 3 h the mixture was heated to 60° C. for 2.5 h. The mixture was cooled to rt and quenched with saturated NaHCO₃ and extracted with EtOAc (×2). The combined organics were concentrated under reduced pressure and the residue purified by silica gel chromatography (0-100% EtOAc/heptane) to give 6′-chloro-1′-(2-(1,1-difluoro-2-methoxyethyl)-6-methylpyrimidin-4-yl)-1′,2′-dihydrospiro[cyclopropane-1,3′-pyrrolo[3,2-c]pyridine] as a white solid (16 mg, 48%). LCMS m/z=367.0 [M+H]⁺

### Preparation 184

### N-(1′-(6-fluoro-4-methylpyridin-2-yl)-1′,2′-dihydrospiro[cyclopropane-1,3′-pyrrolo[3,2-c]pyridin]-6′-yl)acetamide

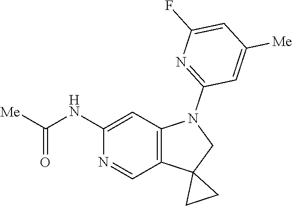 [SMILES: CC(=O)Nc1cc2c(cn1)C1(CC1)CN2c1cc(C)cc(F)n1]

A mixture of N-(1′,2′-dihydrospiro[cyclopropane-1,3′-pyrrolo[3,2-c]pyridin]-6′-yl)acetamide (Preparation 7, 4 g, 19.7 mmol), 2-bromo-6-fluoro-4-methylpyridine (11.22 g, 59 mmol), Cs₂CO₃ (12.82 g, 39.4 mmol) and BrettPhos Pd G3 (892 mg, 0.984 mmol) in dioxane (160 mL) was heated at 100° C. for 2 h. Josiphos (545 mg, 0.984 mmol) was added and the reaction stirred at 90° C. overnight. The mixture was diluted with EtOAc and washed with brine. The organic layer was separated and evaporated to dryness and the residue diluted with MeOH. The solid formed was collected by filtration, washed with MeOH, water, then dried. The resulting solid was dissolved in DCM and filtered through Celite®. The filtrate was then concentrated and recrystallized from MeCN to give N-(1′-(6-fluoro-4-methylpyridin-2-yl)-1′,2′-dihydrospiro[cyclopropane-1,3′-pyrrolo[3,2-c]pyridin]-6′-yl)acetamide as a white powder (3.86 g, 63%). LCMS m/z=313 [M+H]⁺

### Preparation 185

### Ethyl 2-(6-(6′-acetamidospiro[cyclopropane-1,3′-pyrrolo[3,2-c]pyridin]-1′(2′H)-yl)-4-methylpyridin-2-yl)-2,2-difluoroacetate

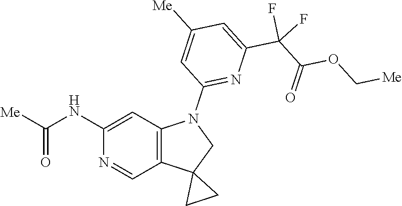 [SMILES: CCOC(=O)C(F)(F)c1cc(C)cc(N2CC3(CC3)c3cnc(NC(C)=O)cc32)n1]

Ethyl 2-(6-(6′-acetamidospiro[cyclopropane-1,3′-pyrrolo[3,2-c]pyridin]-1′(2′H)-yl)-4-methylpyridin-2-yl)-2,2-difluoroacetate was prepared (83 mg, 28%) from ethyl 2-(6-chloro-4-methylpyridin-2-yl)-2,2-difluoroacetate (Preparation 159) and N-(1′,2′-dihydrospiro[cyclopropane-1,3′-pyrrolo[3,2-c]pyridin]-6′-yl)acetamide (Preparation 7) using an analogous method as described for Example 93. LCMS m/z=417 [M+H]⁺.

### Preparation 186

### N-(1′-(6-chloro-2-(1,1-difluoropropyl)pyrimidin-4-yl)-1′,2′-dihydrospiro[cyclopropane-1,3′-pyrrolo[3,2-c]pyridin]-6′-yl)acetamide

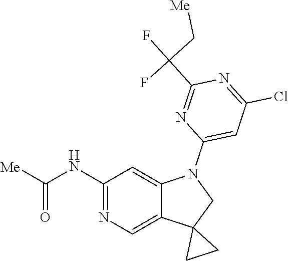 [SMILES: CCC(F)(F)c1nc(Cl)cc(N2CC3(CC3)c3cnc(NC(C)=O)cc32)n1]

N-(1′-(6-Chloro-2-(1,1-difluoropropyl)pyrimidin-4-yl)-1′,2′-dihydrospiro[cyclopropane-1,3′-pyrrolo[3,2-c]pyridin]-6′-yl)acetamide was obtained as a white solid (24 mg, 40%), from 4,6-dichloro-2-(1,1-difluoropropyl)pyrimidine (Preparation 164) and N-(1′,2′-dihydrospiro[cyclopropane-1,3′-pyrrolo[3,2-c]pyridin]-6′-yl)acetamide (Preparation 7), following an analogous method to that described in Preparation 72. LCMS m/z=394.0 [M+H]⁺.

### Preparation 187

### N-(1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridin]-6′-yl)acetamide

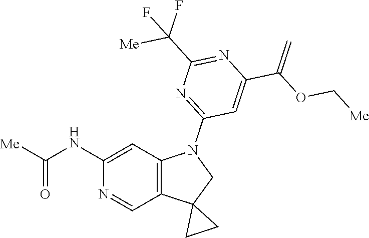 [SMILES: C=C(OCC)c1cc(N2CC3(CC3)c3cnc(NC(C)=O)cc32)nc(C(C)(F)F)n1]

To a mixture of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 2 g, 5.27 mmol) and Pd(PPh₃)₄Cl₂ (370 mg, 0.527 mmol) in DMF (20 mL) was added tributyl(1-ethoxyvinyl)stannane (2.28 g, 6.32 mmol) at rt. The mixture was purged with N₂ and then heated at 80° C. for 30 min. After cooling the mixture was diluted with EtOAc and the solids removed by filtration. The filtrate was stirred with KF-alumina (3 g, 40% w) for 30 min. The mixture was filtered and the filtrate washed with water (3×) and brine. The combined organics were dried and evaporated to dryness under reduced pressure, the resulting solid collected and rinsed with heptane to give N-(1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridin]-6′-yl)acetamide (1.40 g, 64%). LCMS m/z=416 [M+H]⁺.

### Preparation 188

### N-(1′-(6-acetyl-2-(1,1-difluoroethyl)pyrimidin-4-yl)-1′,2′-dihydrospiro[cyclopropane-1,3′-pyrrolo[3,2-c]pyridin]-6′-yl)acetamide

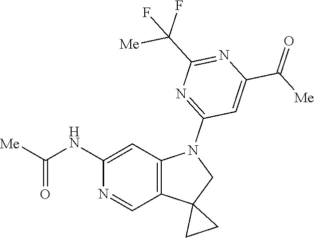 [SMILES: CC(=O)Nc1cc2c(cn1)C1(CC1)CN2c1cc(C(C)=O)nc(C(C)(F)F)n1]

To a solution of N-(1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridin]-6′-yl)acetamide (Preparation 187, 110 mg, 0.265 mmol) in acetone (2 mL) was added HCl (2M, 0.4 mL) and the mixture stirred at rt for 3 h. The reaction mixture was evaporated to dryness under reduced pressure and the residue diluted with EtOAc and made slightly basic with aq. Na₂CO₃. The organics were dried and evaporated to dryness and the residue purified by chromatography on silica gel (0-80% (3:1 EtOAc/EtOH)/heptane (+2% NH₄OH)) to give N-(1′-(6-acetyl-2-(1,1-difluoroethyl)pyrimidin-4-yl)-1′,2′-dihydrospiro[cyclopropane-1,3′-pyrrolo[3,2-c]pyridin]-6′-yl)acetamide (37 mg, 36%). LCMS m/z=388 [M+H]⁺

### Preparation 189

### N-(1′-(6-(chloromethyl)-2-(1,1-difluoroethyl)pyrimidin-4-yl)-1′,2′-dihydrospiro[cyclopropane-1,3′-pyrrolo[3,2-c]pyridin]-6′-yl)acetamide

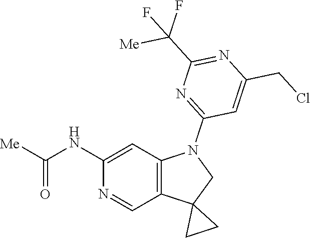 [SMILES: CC(=O)Nc1cc2c(cn1)C1(CC1)CN2c1cc(CCl)nc(C(C)(F)F)n1]

To a solution of N-(1′-(2-(1,1-difluoroethyl)-6-(hydroxymethyl)pyrimidin-4-yl)-1′,2′-dihydrospiro[cyclopropane-1,3′-pyrrolo[3,2-c]pyridin]-6′-yl)acetamide (Example 292, 70 mg, 0.186 mmol) in DCM (2 mL) was added SOCl₂ (222 mg, 1.86 mmol) and the reaction stirred at rt overnight. The reaction mixture was evaporated to dryness under reduced pressure and the residue dissolved in EtOAc and washed with aq. NaHCO₃. The combined organics were dried and concentrated to give N-(1′-(6-(chloromethyl)-2-(1,1-difluoroethyl)pyrimidin-4-yl)-1′,2′-dihydrospiro[cyclopropane-1,3′-pyrrolo[3,2-c]pyridin]-6′-yl)acetamide (67 mg, 91%). LCMS m/z=394 [M+H]⁺.

### Preparation 190

### ethyl 2-(6-(6′-acetamidospiro[cyclopropane-1,3′-pyrrolo[3,2-c]pyridin]-1′(2′H)-yl)-2-(1,1-difluoroethyl)pyrimidin-4-yl)-2-cyanoacetate

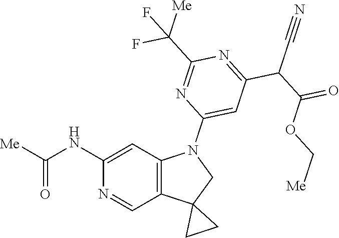 [SMILES: CCOC(=O)C(C#N)c1cc(N2CC3(CC3)c3cnc(NC(C)=O)cc32)nc(C(C)(F)F)n1]

To a solution of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 200 mg, 0.527 mmol) and ethyl 2-cyanoacetate (119 mg, 1.05 mmol) in DMF (4 mL) was added sodium tert-butoxide (101 mg, 1.05 mmol) at rt and the mixture heated at 80° C. for 1 h. Most of the volatiles were removed under reduced pressure and the residue diluted with EtOAc and washed with water (3×). The organic layer was separated, dried and concentrated to give ethyl 2-(6-(6′-acetamidospiro[cyclopropane-1,3′-pyrrolo[3,2-c]pyridin]-1′(2′H)-yl)-2-(1,1-difluoroethyl)pyrimidin-4-yl)-2-cyanoacetate (145 mg, 60%). LCMS m/z=457 [M+H]⁺.

### Preparation 191

### 1-methyl-2-oxabicyclo[2.1.1]hexane-4-carboxamide

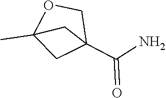

To a solution of 1-methyl-2-oxabicyclo[2.1.1]hexane-4-carboxylic acid (1 g, 7.03 mmol) in DCM (10 mL) under N₂ was added oxalyl chloride (2 M in DCM, 4.2 mL) and DMF (54 uL) and the solution stirred at rt for 1 h. The reaction mixture was concentrated in vacuo, the residue was dissolved in DCM (10 mL) and the solution cooled to 0° C. Ammonia (7 M in methanol, 20 mL) was slowly added and the mixture was stirred at rt for 2 h. The solid formed was filtered off and rinsed with MeOH. The filtrate was concentrated, diluted with MeCN, the solid formed was collected by filtration and washed with cold MeCN to give 1-methyl-2-oxabicyclo[2.1.1]hexane-4-carboxamide (741 mg, 74% yield) as a white solid. LCMS m/z=142 [M+H]⁺

### Preparation 192

### 6-methyl-2-(1-methyl-2-oxabicyclo[2.1.1]hexan-4-yl)pyrimidin-4(3H)-one

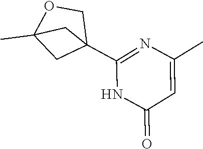

A mixture of NaOMe (2.32 mL, 25% in MeOH) and n-BuOH (3.0 mL) was heated to 105° C. in a 20 mL sealed vial (poked with a needle open to air) while the solvent was removed. To this was slowly added a suspension of methyl (E)-3-aminobut-2-enoate (300 mg, 2.61 mmol) and 1-methyl-2-oxabicyclo[2.1.1]hexane-4-carboxamide (919.6 mg, 6.51 mmol) in n-BuOH (6.0 mL) and MeOH (6.0 mL). The temperature was raised to 110° C. while the removal of the solvent continued and the reaction heated for a further 2 h. The cooled reaction mixture was neutralized with conc. sulfuric acid, the resulting precipitate filtered off and the filtrate was concentrated under reduced pressure. The filtrate was concentrated and purified by chromatography on silica gel (EtOAc/0-60% EtOAc-EtOH 3:1+2% AcOH) to give the title compound (178 mg, 33% yield) as a yellowish oil. LCMS m/z=207 [M+H]⁺

### Preparation 193

### 4-chloro-6-methyl-2-(1-methyl-2-oxabicyclo[2.1.1]hexan-4-yl)pyrimidine

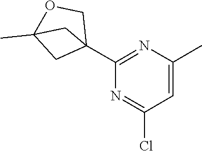

A mixture of 6-methyl-2-(1-methyl-2-oxabicyclo[2.1.1]hexan-4-yl)pyrimidin-4(3H)-one (Preparation 192, 170 mg, 824 umol), DMF (64 uL) and POCl₃ (253 mg, 1.65 mmol) in toluene (5 mL) was stirred at rt for 2 h. The mixture was concentrated under reduced pressure, EtOAc added and the mixture was neutralized with NaHCO₃(aq). The organic layer was separated, washed with brine, then dried and concentrated to give 4-chloro-6-methyl-2-(1-methyl-2-oxabicyclo[2.1.1]hexan-4-yl)pyrimidine (90 mg, 48% yield) as a yellow oil. LCMS m/z=225 [M+H]⁺

### Preparation 194

### 2-oxabicyclo[2.1.1]hexane-4-carboxamide

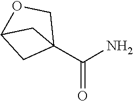

2-Oxabicyclo[2.1.1]hexane-4-carboxamide was obtained as a white solid, 1.86 g, 93.7%, from 2-oxabicyclo[2.1.1]hexane-4-carboxylic acid, following an analogous method to that described in Preparation 191. 1H NMR (MeOD-d4, 400 MHz) δ 4.50 (t, 1H, J=1.1 Hz), 3.81 (s, 2H), 2.12 (td, 2H, J=1.3, 4.9 Hz), 1.90-1.70 (m, 2H).

### Preparation 195

### 2-(2-oxabicyclo[2.1.1]hexan-4-yl)-6-methylpyrimidin-4(3H)-one

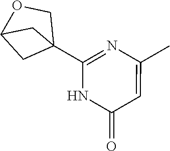

2-(2-Oxabicyclo[2.1.1]hexan-4-yl)-6-methylpyrimidin-4(3H)-one was obtained as a solid, from 2-oxabicyclo[2.1.1]hexane-4-carboxamide and methyl (E)-3-aminobut-2-enoate, following an analogous method to that described in Preparation 192. LCMS m/z=193 [M+H]⁺

### Preparation 196

### 2-(2-oxabicyclo[2.1.1]hexan-4-yl)-4-chloro-6-methylpyrimidine

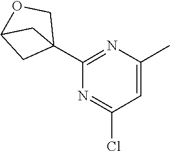

2-(2-Oxabicyclo[2.1.1]hexan-4-yl)-4-chloro-6-methylpyrimidine was obtained as an oil, 715 mg, crude, from 2-(2-oxabicyclo[2.1.1]hexan-4-yl)-6-methylpyrimidin-4(3H)-one, following an analogous method to that described in Preparation 193. LCMS m/z=211 [M+H]⁺

### Preparation 197

### 2-(2-oxabicyclo[2.1.1]hexan-4-yl)-6-ethylpyrimidin-4(3H)-one

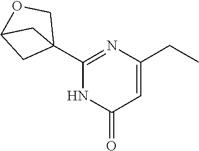

2-(2-Oxabicyclo[2.1.1]hexan-4-yl)-6-ethylpyrimidin-4(3H)-one was obtained as a solid, crude, from methyl (E)-3-aminopent-2-enoate and 2-oxabicyclo[2.1.1]hexane-4-carboxamide, following an analogous method to that described in Preparation 192. LCMS m/z=207 [M+H]⁺

### Preparation 196

### 2-(2-oxabicyclo[2.1.1]hexan-4-yl)-4-chloro-6-ethylpyrimidine

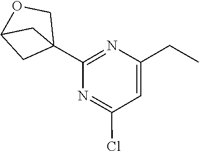

2-(2-Oxabicyclo[2.1.1]hexan-4-yl)-4-chloro-6-ethylpyrimidine was obtained as an oil, from 2-(2-oxabicyclo[2.1.1]hexan-4-yl)-6-ethylpyrimidin-4(3H)-one (Preparation 197), following an analogous method to that described in Preparation 193. LCMS m/z=255 [M+H]⁺.

### Example 1

### N-(1′-(2-(1,1-difluoroethyl)-6-methoxypyrimidin-4-yl)-1′,2′-dihydrospiro[cyclopropane-1,3′-pyrrolo[3,2-c]pyridin]-6′-yl)acetamide

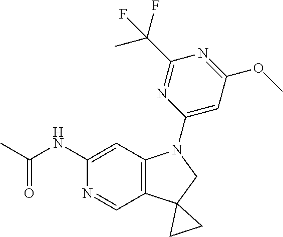 [SMILES: COc1cc(N2CC3(CC3)c3cnc(NC(C)=O)cc32)nc(C(C)(F)F)n1]

To a solution of 6′-chloro-1′-(2-(1,1-difluoroethyl)-6-methoxypyrimidin-4-yl)-1′,2′-dihydrospiro[cyclopropane-1,3′-pyrrolo[3,2-c]pyridine] (Preparation 58, 150 mg, 0.425 mmol), acetamide (50.2 mg, 0.850 mmol) and Cs₂CO₃ (416 mg, 1.28 mmol) in dioxane (5 mL) was added BrettPhos Pd G₃ (38.6 mg, 0.425 mmol) at 25° C. The reaction mixture was stirred at 100° C. for 1 h under N₂. The mixture was diluted with water (50 mL) and extracted with EtOAc (30 mL×3). The combined organic layers were washed with brine (20 mL), dried over Na₂SO₄, filtered and concentrated under vacuum to give the crude, which was purified by prep-HPLC (Column: Waters Xbridge BEH C18 100*25 mm*5 um; Condition: water (Formic Acid)-MeCN; Begin B: 17; End B: 37; Gradient Time (min): 22; Flow Rate (ml/min): 25) to give N-(1′-(2-(1,1-difluoroethyl)-6-methoxypyrimidin-4-yl)-1′,2′-dihydrospiro[cyclopropane-1,3′-pyrrolo[3,2-c]pyridin]-6′-yl)acetamide (29.5 mg, 18.5% yield) as a white solid. LCMS m/z=376.1 [M+H]⁺. ¹H NMR: (500 MHz, DMSO-d₆) δ: ppm 10.88 (s, 1H), 8.71 (s, 1H), 7.70 (s, 1H), 6.29 (s, 1H), 4.17 (s, 2H), 3.99 (s, 3H), 2.20-2.11 (m, 6H), 1.22-1.20 (m, 4H).

### Examples 2 to 9

The compounds in the following table were prepared from the appropriate pyrrolo[3,2-c]pyridine and acetamide, following a similar procedure to that described in Example 1. The crude material was purified by Prep-HPLC (Column: Boston Prime C18 150*25 mm*5 um; Condition: water (NH₃·H₂O+NH₄HCO₃)-MeCN; Gradient Time (min): 10; Flow Rate (ml/min): 25) using an appropriate gradient.

| Example | |
|---|---|
| No | Name, Structure, Starting material, Data |
| 2 | N-(1′-(4-(1,1-difluoroethyl)-6-methoxypyrimidin-2-yl)-1′,2′- |
| | dihydrospiro[cyclopropane-1,3′-pyrrolo[3,2-c]pyridin]-6′-yl)acetamide |
| | 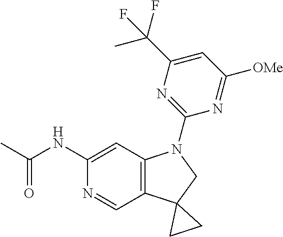 [SMILES: COc1cc(C(C)(F)F)nc(N2CC3(CC3)c3cnc(NC(C)=O)cc32)n1] |
| | SM: 6′-chloro-1′-(4-(1,1-difluoroethyl)-6-methoxypyrimidin-2-yl)-1′,2′- |
| | dihydrospiro[cyclopropane-1,3′-pyrrolo[3,2-c]pyridine] (Preparation 74) |
| | 6 mg, 14.1% yield, yellow solid. |
| | LCMS m/z = 376.1 [M + H]⁺. ¹H NMR (400 MHz, CDCl₃) δ: ppm 9.01 (s, |
| | 1H), 7.86 (s, 1H), 7.47 (s, 1H), 6.59 (s, 1H), 4.28 (s, 2H), 4.18 (s, 3H), 2.18 |
| | (s, 3H), 2.05-1.96 (m, 3H), 1.15 (s, 4H). |
| 3 | N-(1′-(2-(oxetan-3-yl)pyrimidin-4-yl)-1′,2′-dihydrospiro[cyclopropane-1,3′- |
| | pyrrolo[3,2-c]pyridin]-6′-yl)acetamide |
| | 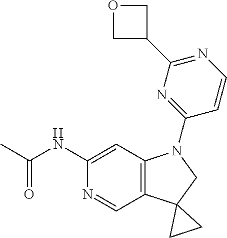 [SMILES: CC(=O)Nc1cc2c(cn1)C1(CC1)CN2c1ccnc(C2COC2)n1] |
| | SM: 6′-chloro-1′-(2-(oxetan-3-yl)pyrimidin-4-yl)-1′,2′- |
| | dihydrospiro[cyclopropane-1,3′-pyrrolo[3,2-c]pyridine] (Preparation 61) |
| | 38.0 mg, 50.6% yield, white solid. |
| | LCMS m/z = 338.1 [M + H]⁺. ¹H NMR (400 MHz, CDCl₃) δ: ppm 9.04 (s, |
| | 1H), 8.51 (d, J = 5.5 Hz, 1H), 8.31 (s, 1H), 7.51 (s, 1H), 6.61 (d, J = 6.0 Hz, |
| | 1H), 5.17-5.11 (m, 4H), 4.56-4.50 (m, 1H), 4.12 (s, 2H), 2.20 (s, 3H), 1.21- |
| | 1.16 (m, 4H). |
| 4 | N-(1′-(2-(1,1-difluoroethyl)-6-(2-methoxyethoxy)pyrimidin-4-yl)-1′,2′- |
| | dihydrospiro[cyclopropane-1,3′-pyrrolo[3,2-c]pyridin]-6′-yl)acetamide |
| | 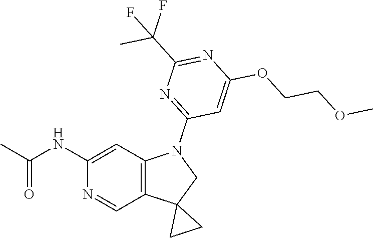 [SMILES: COCCOc1cc(N2CC3(CC3)c3cnc(NC(C)=O)cc32)nc(C(C)(F)F)n1] |
| | SM: 6′-chloro-1′-(2-(1,1-difluoroethyl)-6-(2-methoxyethoxy)pyrimidin-4- |
| | yl)-1′,2′-dihydrospiro[cyclopropane-1,3′-pyrrolo[3,2-c]pyridine] |
| | (Preparation 127) |
| | 15.6 mg, 29.5% yield, white solid. |
| | LCMS m/z = 420.1 [M + H]⁺. ¹H NMR (500 MHz, CDCl₃) δ: ppm 9.04 (s, |
| | 1H), 7.93 (s, 1H), 7.45 (s, 1H), 6.02 (s, 1H), 4.62-4.59 (m, 2H), 4.01 (s, 2H), |
| | 3.76-3.73 (m, 2H), 3.45 (s, 3H), 2.20 (s, 3H), 2.18 (t, J = 18.5Hz, 3H), 1.20- |
| | 1.17 (m, 2H), 1.15-1.12 (m, 2H). |
| 5 | N-(1′-(2-(1,1-difluoroethyl)-6-(3-methoxycyclobutoxy)pyrimidin-4-yl)-1′,2′- |
| | dihydrospiro[cyclopropane-1,3′-pyrrolo[3,2-c]pyridin]-6′-yl)acetamide |
| | 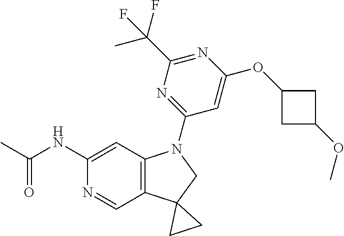 [SMILES: COC1CC(Oc2cc(N3CC4(CC4)c4cnc(NC(C)=O)cc43)nc(C(C)(F)F)n2)C1] |
| | SM: 6′-chloro-1′-(2-(1,1-difluoroethyl)-6-(3- |
| | methoxycyclobutoxy)pyrimidin-4-yl)-1′,2′-dihydrospiro[cyclopropane-1,3′- |
| | pyrrolo[3,2-c]pyridine] (Preparation 128) |
| | 24.0 mg, 30.4% yield, white solid. |
| | LCMS m/z = 446.1 [M + H]⁺. ¹H NMR (500 MHz, CDCl₃) δ: ppm 8.98-9.01 |
| | (m, 1H), 8.02 (s, 1H), 7.48 (s, 1H), 5.97-5.95 (m, 1H), 5.44-5.41 (m, 0.4H), |
| | 5.00-4.96 (m, 0.7H), 4.14-4.12 (m, 0.4H), 4.05-4.02 (m, 2H), 3.67-3.65 (m, |
| | 0.7H), 3.28-3.26 (m, 3H), 2.95-2.90 (m, 1.4H), 2.53-2.49 (m, 0.8H), 2.45- |
| | 2.38 (m, 0.8H), 2.19-2.18 (m, 3H), 2.17-2.14 (m, 4H), 1.18-1.11 (m, 2H), |
| | 1.15-1.14 (m, 2H). |
| 6 | N-(1′-(6-cyclopropoxy-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | 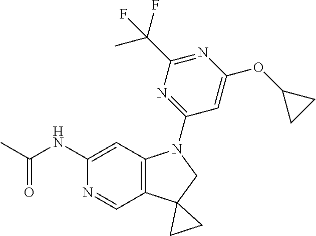 [SMILES: CC(=O)Nc1cc2c(cn1)C1(CC1)CN2c1cc(OC2CC2)nc(C(C)(F)F)n1] |
| | SM: 6′-chloro-1′-(6-cyclopropoxy-2-(1,1-difluoroethyl)pyrimidin-4-yl)- |
| | 1′,2′-dihydrospiro[cyclopropane-1,3′-pyrrolo[3,2-c]pyridine] (Preparation |
| | 129) |
| | 24.0 mg, 37.7% yield, white solid. |
| | LCMS m/z = 402.1 [M + H]⁺. ¹H NMR (400 MHz, CDCl₃) δ: ppm 9.00 (s, |
| | 1H), 8.00 (s, 1H), 7.51 (s, 1H), 6.20 (s, 1H), 4.35-4.30 (m, 1H), 4.12 (s, 2H), |
| | 2.23-2.13 (m, 6H), 1.21-1.20 (m, 2H), 1.19-1.17 (m, 2H), 0.91-0.85 (m, 4H). |
| 7 | N-(1′-(6-(1,1-difluoroethyl)-5-fluoropyridin-2-yl)-1′,2′- |
| | dihydrospiro[cyclopropane-1,3′-pyrrolo[3,2-c]pyridin]-6′-yl)acetamide |
| | 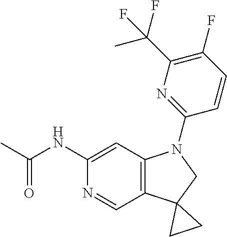 [SMILES: CC(=O)Nc1cc2c(cn1)C1(CC1)CN2c1ccc(F)c(C(C)(F)F)n1] |
| | SM: 6′-chloro-1′-(6-(1,1-difluoroethyl)-5-fluoropyridin-2-yl)-1′,2′- |
| | dihydrospiro[cyclopropane-1,3′-pyrrolo[3,2-c]pyridine] (Preparation 136) |
| | 20.0 mg, 25.0% yield, white solid. |
| | LCMS m/z = 363.1 [M + H]⁺. ¹H NMR (500 MHz, CDCl₃) δ: ppm 8.67 (s, |
| | 1H), 8.27 (s, 1H), 7.54-7.50 (m, 1H), 7.46 (s, 1H), 6.94 (d, J = 8.5 Hz, 1H), |
| | 4.12 (s, 2H), 2.25-2.17 (m, 6H), 1.17-1.16 (m, 2H), 1.15-1.13 (m, 2H). |
| 8 | N-(1′-(6-(1,1-difluoroethyl)-5-methoxypyridin-2-yl)-1′,2′- |
| | dihydrospiro[cyclopropane-1,3′-pyrrolo[3,2-c]pyridin]-6′-yl)acetamide |
| | 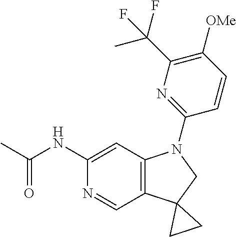 [SMILES: COc1ccc(N2CC3(CC3)c3cnc(NC(C)=O)cc32)nc1C(C)(F)F] |
| | SM: 6′-chloro-1′-(6-(1,1-difluoroethyl)-5-methoxypyridin-2-yl)-1′,2′- |
| | dihydrospiro[cyclopropane-1,3′-pyrrolo[3,2-c]pyridine] (Preparation 65) |
| | 60.0 mg, 43.4% yield, white solid. |
| | LCMS m/z = 375.1 [M + H]⁺. ¹H NMR (500 MHz, CDCl₃) δ: ppm 8.62 (s, |
| | 1H), 8.52 (s, 1H), 7.43-7.40 (m, 2H), 7.02 (d, J = 9.0 Hz, 1H), 4.12 (s, 2H), |
| | 3.90 (s, 3H), 2.20-2.11 (m, 6H), 1.25-1.15 (m, 4H). |
| 9A | N-(1′-(6-(1,1-difluoroethyl)-4-methoxypyridin-2-yl)-1′,2′- |
| | dihydrospiro[cyclopropane-1,3′-pyrrolo[3,2-c]pyridin]-6′-yl)acetamide |
| | 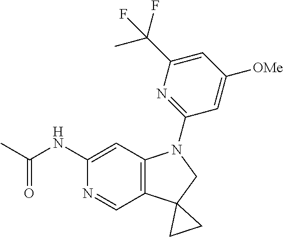 [SMILES: COc1cc(N2CC3(CC3)c3cnc(NC(C)=O)cc32)nc(C(C)(F)F)c1] |
| | SM: 6′-chloro-1′-(6-(1,1-difluoroethyl)-4-methoxypyridin-2-yl)-1′,2′- |
| | dihydrospiro[cyclopropane-1,3′-pyrrolo[3,2-c]pyridine] (Preparation 62) |
| | 68 mg, 63.9% yield, white solid. |
| | LCMS m/z = 375.1 [M + H]⁺. ¹H NMR (500 MHz, CDCl₃) δ: ppm 8.82 (s, |
| | 1H), 8.16 (s, 1H), 7.43 (s, 1H), 6.87 (s, 1H), 6.35 (s, 1H), 4.09 (s, 2H), 3.92 |
| | (s, 3H), 2.22-2.11 (m, 6H), 1.17-1.09 (m, 4H). |

A = DMF was used as the reaction solvent

### Example 10

### N-(1′-(2-(1,1-difluoroethyl)-6-(methylamino)pyrimidin-4-yl)-1′,2′-dihydrospiro[cyclopropane-1,3′-pyrrolo[3,2-c]pyridin]-6′-yl)acetamide

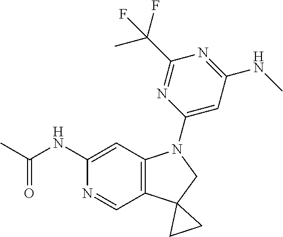 [SMILES: CNc1cc(N2CC3(CC3)c3cnc(NC(C)=O)cc32)nc(C(C)(F)F)n1]

A mixture of 6-(6′-chlorospiro[cyclopropane-1,3′-pyrrolo[3,2-c]pyridin]-1′(2′H)-yl)-2-(1,1-difluoroethyl)-N-methylpyrimidin-4-amine (Preparation 131, 50 mg, 142.1 μmol), acetamide (41.98 mg, 710.7 μmol), Cs₂CO₃ (92.62 mg, 284.3 μmol) and Brettphos Pd G3 (6.44 mg, 7.11 μmol) in dioxane (2 mL) was purged with N₂, then heated at 110° C. in a sealed tube for 1 h. The cooled mixture was filter and the resulting solid was dry loaded on silica gel and purified by chromatography on silica gel (0-80% EtOAc-EtOH 3:1 with 2% NH₄OH in heptane) to give N-(1′-(2-(1,1-difluoroethyl)-6-(methylamino)pyrimidin-4-yl)-1′,2′-dihydrospiro[cyclopropane-1,3′-pyrrolo[3,2-c]pyridin]-6′-yl)acetamide (40 mg, 75.2% yield) as a white powder. LCMS m/z=375.2 [M+H]^{+ 1}H NMR (400 MHz, MeOH-d₄) δ 9.00-8.70 (m, 1H), 7.52 (s, 1H), 5.69 (s, 1H), 4.08 (s, 2H), 2.92 (s, 3H), 2.16 (s, 3H), 2.10-2.00 (m, 3H), 1.18 (br d, 4H, J=2.5 Hz)

### Example 11

### N-(1′-(2-(1,1-difluoroethyl)-6-ethylpyrimidin-4-yl)-1′,2′-dihydrospiro[cyclopropane-1,3′-pyrrolo[3,2-c]pyridin]-6′-yl)acetamide

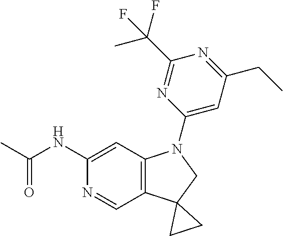 [SMILES: CCc1cc(N2CC3(CC3)c3cnc(NC(C)=O)cc32)nc(C(C)(F)F)n1]

A mixture of 6′-chloro-1′-(2-(1,1-difluoroethyl)-6-ethylpyrimidin-4-yl)-1′,2′-dihydrospiro[cyclopropane-1,3′-pyrrolo[3,2-c]pyridine] (Preparation 137, 35 mg, 100 μmol), acetamide (30 mg, 500 μmol), Cs₂CO₃ (65 mg, 200 μmol) and BrettPhos Pd G3 (4.5 mg, 5 μmol) in dioxane (1 mL) was purged with N₂, then heated at 110° C. in a sealed tube for 1 h. The cooled mixture was filtered, the filtrate concentrated and the crude was purified by chromatography on silica gel (0-70% EtOAc-EtOH 3:1 with 2% NH₄OH in heptane) to give N-(1′-(2-(1,1-difluoroethyl)-6-ethylpyrimidin-4-yl)-1′,2′-dihydrospiro[cyclopropane-1,3′-pyrrolo[3,2-c]pyridin]-6′-yl)acetamide (29 mg, 77% yield) as a white powder. LCMS m/z= 374.2 [M+H]⁺. ¹H NMR: (400 MHz, CDCl₃) δ: ppm 9.03 (br s, 1H), 8.14 (br s, 1H), 7.51 (s, 1H), 6.59 (s, 1H), 4.14 (s, 2H), 2.84 (q, 2H, J=7.6 Hz), 2.30-2.10 (m, 6H), 1.35 (t, 3H, J=7.5 Hz), 1.20-1.10 (m, 4H).

### Example 12

### N-(1′-(6-(1,1-difluoroethyl)pyridin-2-yl)-1′,2′-dihydrospiro[cyclopropane-1,3′-pyrrolo[3,2-c]pyridin]-6′-yl)acetamide

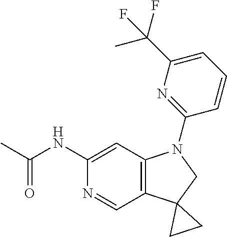 [SMILES: CC(=O)Nc1cc2c(cn1)C1(CC1)CN2c1cccc(C(C)(F)F)n1]

A mixture of 6′-chloro-1′-(6-(1,1-difluoroethyl)pyridin-2-yl)-1′,2′-dihydrospiro[cyclopropane-1,3′-pyrrolo[3,2-c]pyridine] (Preparation 77, 70 mg, 217 μmol), acetamide (64 mg, 1.1 mmol), Cs₂CO₃ (142 mg, 435 μmol) and BrettPhos Pd G3 (9.9 mg, 11 μmol) in dioxane (2 mL) was purged with N₂, then heated at 110° C. in a sealed tube for 1 h. The cooled mixture was filtered and the crude was purified by chromatography on silica gel (0-80% EtOAc-EtOH 3:1 with 2% NH₄OH in heptane) to give N-[1-[6-(1,1-difluoroethyl)-2-pyridyl]spiro[2H-pyrrolo[3,2-c]pyridine-3,1′-cyclopropane]-6-yl]acetamide (37 mg, 49% yield) as a white powder. LCMS m/z=345.1 [M+H]⁺

1H NMR (CDCl₃, 400 MHz) δ 8.86 (s, 1H), 8.05 (br s, 1H), 7.78 (t, 1H, J=7.9 Hz), 7.45 (s, 1H), 7.29 (d, 1H, J=7.5 Hz), 6.90-6.80 (m, 1H), 4.14 (s, 2H), 2.30-2.10 (m, 6H), 1.20-1.10 (m, 4H)

### Examples 13 to 71

the compounds in the following table were prepared from acetamide and the appropriate chloride, following a similar procedure to that described in Example 12.

| Example | |
|---|---|
| No | Name, Structure, Starting material (SM), Data |
| 13 | N-(1′-(2-(1,1-difluoroethyl)pyrimidin-4-yl)-1′,2′-dihydrospiro[cyclopropane- |
| | 1,3′-pyrrolo[3,2-c]pyridin]-6′-yl)acetamide |
| | 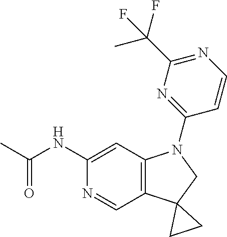 [SMILES: CC(=O)Nc1cc2c(cn1)C1(CC1)CN2c1ccnc(C(C)(F)F)n1] |
| | SM: 6′-chloro-1′-(2-(1,1-difluoroethyl)pyrimidin-4-yl)-1′,2′- |
| | dihydrospiro[cyclopropane-1,3′-pyrrolo[3,2-c]pyridine] (Preparation 85) |
| | 82 mg, 95% yield, white powder. |
| | LCMS m/z = 346.1 [M + H]⁺. ¹H NMR: (500 MHz, CDCl₃) δ: ppm 9.06 (br |
| | s, 1H), 8.63 (d, 1H, J = 6.0 Hz), 8.25 (br s, 1H), 7.52 (s, 1H), 6.75 (d, |
| | 1H, J = 6.0 Hz), 4.15 (s, 2H), 2.30-2.20 (m, 6H), 1.30-1.20 (m, 4H). |
| 14 | N-(1′-(4-(1,1-difluoroethyl)pyrimidin-2-yl)-1′,2′-dihydrospiro[cyclopropane- |
| | 1,3′-pyrrolo[3,2-c]pyridin]-6′-yl)acetamide |
| | 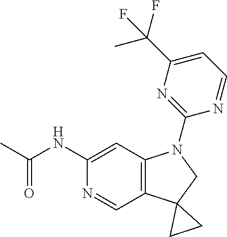 [SMILES: CC(=O)Nc1cc2c(cn1)C1(CC1)CN2c1nccc(C(C)(F)F)n1] |
| | SM: 6′-chloro-1′-(4-(1,1-difluoroethyl)pyrimidin-2-yl)-1′,2′- |
| | dihydrospiro[cyclopropane-1,3′-pyrrolo[3,2-c]pyridine] (Preparation 86) |
| | 27 mg, 63% yield, white powder. |
| | LCMS m/z = 346.1 [M + H]⁺. ¹H NMR: (400 MHz, CDCl₃) δ: ppm 9.08 (br |
| | s, 1H), 8.80-8.60 (m, 1H), 8.29 (br s, 1H), 7.49 (s, 1H), 7.17 (d, 1H, J = 5.0 |
| | Hz), 4.31 (s, 2H), 2.30-2.10 (m, 6H), 1.17 (s, 4H). |
| 15 | N-(1′-(6-acetamido-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | 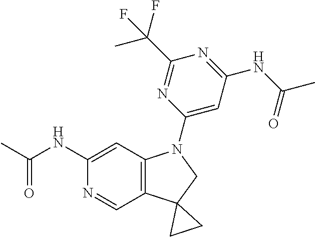 [SMILES: CC(=O)Nc1cc2c(cn1)C1(CC1)CN2c1cc(NC(C)=O)nc(C(C)(F)F)n1] |
| | SM: 6′-chloro-1′-(2-(1,1-difluoroethyl)-6-(methylsulfonyl)pyrimidin-4-yl)- |
| | 1′,2′-dihydrospiro[cyclopropane-1,3′-pyrrolo[3,2-c]pyridine (Preparation |
| | 147) |
| | 4 mg, 20% yield, white powder. |
| | LCMS m/z = 403.2 [M + H]⁺. ¹H NMR: (500 MHz, MeOH-d₄) δ: ppm 9.10- |
| | 8.90 (m, 1H), 7.58 (d, 2H, J = 6.7 Hz), 4.15 (s, 2H), 2.19 (s, 3H), 2.17 (s, |
| | 3H), 2.11 (t, 3H, J = 18.8 Hz), 1.30-1.20 (m, 4H). |
| 16 | N-(1′-(6-(tetrahydrofuran-3-yl)pyridin-2-yl)-1′,2′- |
| | dihydrospiro[cyclopropane-1,3′-pyrrolo[3,2-c]pyridin]-6′-yl)acetamide |
| | 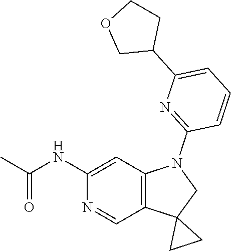 [SMILES: CC(=O)Nc1cc2c(cn1)C1(CC1)CN2c1cccc(C2CCOC2)n1] |
| | SM: 6′-chloro-1′-(6-(tetrahydrofuran-3-yl)pyridin-2-yl)-1′,2′- |
| | dihydrospiro[cyclopropane-1,3′-pyrrolo[3,2-c]pyridine] (Preparation 78) |
| | 43 mg, LCMS m/z = 351.1 [M + H]⁺. ¹H NMR: (400 MHz, CDCl₃) δ: ppm |
| | 8.82 (br s, 1H), 8.24 (br s, 1H), 7.60-7.50 (m, 1H), 7.42 (s, 1H), 6.84 (d, |
| | 1H, J = 7.5 Hz), 6.72 (d, 1H, J = 8.3 Hz), 4.25 (t, 1H, J = 7.9 Hz), 4.20-4.10 (m, |
| | 3H), 4.0-3.9 (m, 2H), 3.60 (quin, 1H, J = 7.9 Hz), 2.50-2.40 (m, 2H), 2.19 (s, |
| | 3H), 1.20-1.10 (m, 4H). |
| 17 | N-(1′-(2-(2-fluoropropan-2-yl)pyrimidin-4-yl)-1′,2′- |
| | dihydrospiro[cyclopropane-1,3′-pyrrolo[3,2-c]pyridin]-6′-yl)acetamide |
| | 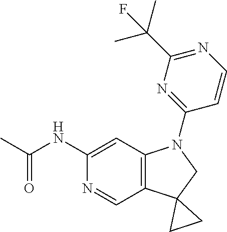 [SMILES: CC(=O)Nc1cc2c(cn1)C1(CC1)CN2c1ccnc(C(C)(C)F)n1] |
| | SM: 6′-chloro-1′-(2-(2-fluoropropan-2-yl)pyrimidin-4-yl)-1′,2′- |
| | dihydrospiro[cyclopropane-1,3′-pyrrolo[3,2-c]pyridine] (Preparation 87) |
| | 48 mg, LCMS m/z = 342.2 [M + H]⁺. ¹H NMR: (400 MHz, CDCl₃) δ: ppm |
| | 9.20-8.90 (m, 1H), 8.58 (d, 1H, J = 5.8 Hz), 8.23 (br s, 1H), 7.50 (s, 1H), 6.58 |
| | (d, 1H, J = 6.0 Hz), 4.11 (s, 2H), 2.22 (s, 3H), 2.00-1.90 (m, 6H), 1.20-1.20 |
| | (m, 4H). |
| 18 | N-(1′-(6-(3-methoxytetrahydrofuran-3-yl)pyridin-2-y1)-1′,2′- |
| | dihydrospiro[cyclopropane-1,3′-pyrrolo[3,2-c]pyridin]-6′-yl)acetamide |
| | 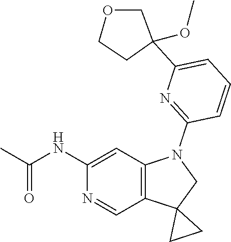 [SMILES: COC1(c2cccc(N3CC4(CC4)c4cnc(NC(C)=O)cc43)n2)CCOC1] |
| | SM: 6′-chloro-1′-(6-(3-methoxytetrahydrofuran-3-yl)pyridin-2-yl)-1′,2′- |
| | dihydrospiro[cyclopropane-1,3′-pyrrolo[3,2-c]pyridine] (Preparation 88) |
| | 40 mg, LCMS m/z = 381.2 [M + H]⁺. ¹H NMR: (400 MHz, CDCl₃) δ: ppm |
| | 8.70 (s, 1H), 8.34 (br s, 1H), 7.80-7.70 (m, 1H), 7.42 (s, 1H), 7.17 (d, |
| | 1H, J = 7.3 Hz), 6.80 (d, 1H, J = 8.0 Hz), 4.30-4.00 (m, 6H), 3.27 (s, 3H), 2.82 |
| | (td, 1H, J = 8.6, 13.2 Hz), 2.48 (dtd, 1H, J = 1.1, 5.5, 13.2 Hz), 2.19 (s, 3H), |
| | 1.20-1.10 (m, 4H). |
| 19 | N-(1′-(6-(3-hydroxytetrahydrofuran-3-yl)pyridin-2-yl)-1′,2′- |
| | dihydrospiro[cyclopropane-1,3′-pyrrolo[3,2-c]pyridin]-6′-yl)acetamide |
| | 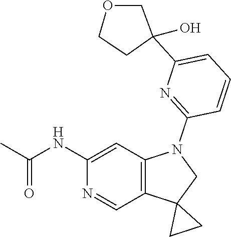 [SMILES: CC(=O)Nc1cc2c(cn1)C1(CC1)CN2c1cccc(C2(O)CCOC2)n1] |
| | SM: 3-(6-(6′-chlorospiro[cyclopropane-1,3′-pyrrolo[3,2-c]pyridin]-1′(2′H)- |
| | yl)pyridin-2-yl)tetrahydrofuran-3-ol (Preparation 89) |
| | 7 mg, LCMS m/z = 367.2 [M + H]⁺. ¹H NMR: (400 MHz, MeOH-d₄) δ: ppm |
| | δ 8.74 (s, 1H), 7.77 (t, 1H, J = 8.0 Hz), 7.48 (s, 1H), 7.36 (d, 1H, J = 7.5 Hz), |
| | 6.80-6.80 (m, 1H), 4.30-4.10 (m, 5H), 3.92 (dd, 1H, J = 1.1, 8.9 Hz), 2.90 (td, |
| | 1H, J = 9.2, 12.8 Hz), 2.20-2.10 (m, 4H), 1.20-1.10 (m, 4H). |
| 20 | N-(1′-(2-(1,1-difluoroethyl)-6-(dimethylamino)pyrimidin-4-yl)-1′,2′- |
| | dihydrospiro[cyclopropane-1,3′-pyrrolo[3,2-c]pyridin]-6′-yl)acetamide |
| | 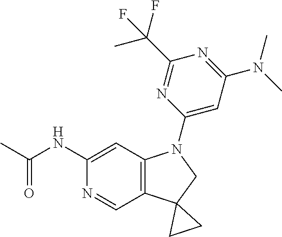 [SMILES: CC(=O)Nc1cc2c(cn1)C1(CC1)CN2c1cc(N(C)C)nc(C(C)(F)F)n1] |
| | SM: 6-(6′-chlorospiro[cyclopropane-1,3′-pyrrolo[3,2-c]pyridin]-1′(2′H)-yl)- |
| | 2-(1,1-difluoroethyl)-N,N-dimethylpyrimidin-4-amine (Preparation 130) |
| | 24 mg, LCMS m/z = 389.2 [M + H]⁺. ¹H NMR: (400 MHz, CDCl₃) δ: ppm |
| | 9.0-8.7 (m, 1H), 8.16 (br s, 1H), 7.44 (s, 1H), 5.75 (s, 1H), 4.11 (s, 2H), 3.16 |
| | (s, 6H), 2.20-2.10 (m, 6H), 1.20-1.10 (m, 4H). |
| 21 | N-(1′-(6-cyclopropyl-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | 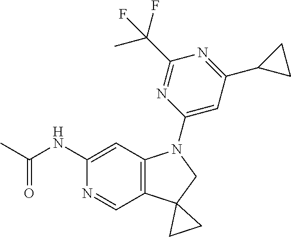 [SMILES: CC(=O)Nc1cc2c(cn1)C1(CC1)CN2c1cc(C2CC2)nc(C(C)(F)F)n1] |
| | SM: 6′-chloro-1′-(6-cyclopropyl-2-(1,1-difluoroethyl)pyrimidin-4-yl)-1′,2′- |
| | dihydrospiro[cyclopropane-1,3′-pyrrolo[3,2-c]pyridine] (Preparation 139)20 |
| | mg, LCMS m/z = 386.2 [M + H]⁺. ¹H NMR: (400 MHz, CDCl₃) δ: ppm 9.00 |
| | (br s, 1H), 8.09 (br s, 1H), 7.50 (s, 1H), 6.57 (s, 1H), 4.13 (s, 2H), 2.21 (s, |
| | 3H), 2.14 (t, 3H, J = 18.8 Hz), 2.10-2.00 (m, 1H), 1.23-1.17 (m, 6H), 1.10- |
| | 1.00 (m, 2H). |
| 22 | N-(1′-(2-(1,1-difluoroethyl)-6-(oxetan-3-yl)pyrimidin-4-yl)-1′,2′- |
| | dihydrospiro[cyclopropane-1,3′-pyrrolo[3,2-c]pyridin]-6′-yl)acetamide |
| | 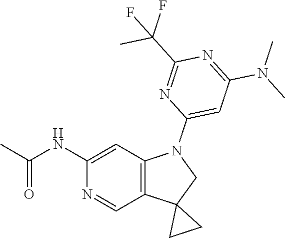 [SMILES: CC(=O)Nc1cc2c(cn1)C1(CC1)CN2c1cc(N(C)C)nc(C(C)(F)F)n1] |
| | SM: 6′-chloro-1′-(2-(1,1-difluoroethyl)-6-(oxetan-3-yl)pyrimidin-4-yl)-1′,2′- |
| | dihydrospiro[cyclopropane-1,3′-pyrrolo[3,2-c]pyridine] (Preparation 141)6 |
| | mg, LCMS m/z = 402.2 [M + H]⁺. ¹H NMR: (400 MHz, CDCl₃) δ: ppm 9.20- |
| | 8.90 (m, 1H), 8.30-8.00 (m, 1H), 7.52 (s, 1H), 6.80 (s, 1H), 5.09 (dd, |
| | 2H, J = 6.1, 8.4 Hz), 4.93 (t, 2H, J = 6.3 Hz), 4.37 (tt, 1H, J = 6.6, 8.4 Hz), 4.18 |
| | (s, 2H), 2.30-2.20 (m, 6H), 1.23-1.20 (m, 4H). |
| 23 | N-(1′-(2-(1, 1-difluoroethyl)-6-(3-methoxycyclobutyl)pyrimidin-4-yl)-1′,2′- |
| | dihydrospiro[cyclopropane-1,3′-pyrrolo[3,2-c]pyridin]-6′-yl)acetamid |
| | 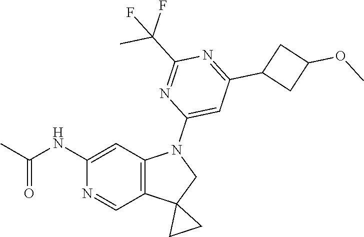 [SMILES: COC1CC(c2cc(N3CC4(CC4)c4cnc(NC(C)=O)cc43)nc(C(C)(F)F)n2)C1] |
| | SM: 6′-chloro-1′-(2-(1,1-difluoroethyl)-6-(3-methoxycyclobutyl)pyrimidin- |
| | 4-y1)-1′,2′-dihydrospiro[cyclopropane-1,3′-pyrrolo[3,2-c]pyridine] |
| | (Preparation 142) |
| | 18 mg, LCMS m/z = 430.2 [M + H]⁺. ¹H NMR: (400 MHz, CDCl₃) δ: ppm |
| | 9.20-8.90 (m, 1H), 8.20-8.00 (m, 1H), 7.51 (s, 1H), 6.60 (br s, 1H), 4.14 (s, |
| | 2H), 4.30-4.10 (m, 1H), 4.00-3.50 (m, 1H), 3.30 (s, 3H), 2.40-2.30 (m, 4H), |
| | 2.30-2.10 (m, 7H), 1.23-1.15 (m, 4H). |
| 24 | N-(1′-(6-cyclobutyl-2-(1,1-difluoroethyl)pyrimidin-4-yl)-1′,2′- |
| | dihydrospiro[cyclopropane-1,3'-pyrrolo[3,2-c]pyridin]-6'-yl)acetamide |
| | 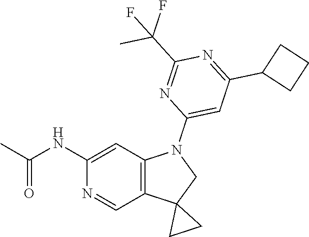 [SMILES: CC(=O)Nc1cc2c(cn1)C1(CC1)CN2c1cc(C2CCC2)nc(C(C)(F)F)n1] |
| | SM: 6′-chloro-1′-(6-cyclobutyl-2-(1,1-difluoroethyl)pyrimidin-4-yl)-1′,2′- |
| | dihydrospiro[cyclopropane-1,3′-pyrrolo[3,2-c]pyridine] (Preparation 140) |
| | 41 mg, LCMS m/z = 400.2 [M + H]⁺. ¹H NMR: (400 MHz, CDCl₃) δ: ppm |
| | 9.10-9.00 (m, 1H), 8.23 (br s, 1H), 7.49 (s, 1H), 6.61 (s, 1H), 4.16 (s, 2H), |
| | 3.67 (quin, 1H, J = 8.5 Hz), 2.40-2.30 (m, 4H), 2.30-2.10 (m, 6H), 2.13-2.03 |
| | (m, 1H), 2.00-1.90 (m, 1H), 1.20 (br d, 4H, J = 4.0 Hz). |
| 25 | N-(1′-(2-(1,1-difluoroethyl)-6-(tetrahydrofuran-3-yl)pyrimidin-4-yl)-1′,2′- |
| | dihydrospiro[cyclopropane-1,3′-pyrrolo[3,2-c]pyridin]-6′-yl)acetamide |
| | 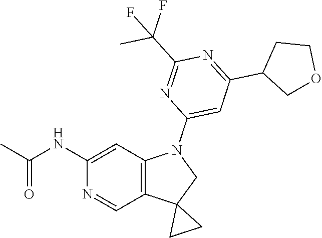 [SMILES: CC(=O)Nc1cc2c(cn1)C1(CC1)CN2c1cc(C2CCOC2)nc(C(C)(F)F)n1] |
| | SM: 6′-chloro-1′-(2-(1,1-difluoroethyl)-6-(tetrahydrofuran-3-yl)pyrimidin-4- |
| | y1)-1′,2′-dihydrospiro[cyclopropane-1,3′-pyrrolo[3,2-c]pyridine] |
| | (Preparation 144) |
| | 37 mg, LCMS m/z = 416.2 [M + H]⁺. ¹H NMR: (400 MHz, MeOH-d₄) δ: |
| | ppm 9.10-8.90 (m, 1H), 7.60 (s, 1H), 6.79 (s, 1H), 4.18 (s, 2H), 4.20-4.10 |
| | (m, 1H), 4.08 (dt, 1H, J = 5.3, 8.2 Hz), 4.00-3.90 (m, 2H), 3.61 (td, |
| | 1H, J = 7.3, 15.2 Hz), 2.40-2.30 (m, 1H), 2.30-2.20 (m, 1H), 2.20-2.10 (m, |
| | 6H), 1.30-1.20 (m, 4H). |
| 26 | N-(1′-(2-(1, 1-difluoroethyl)-6-(tetrahydrofuran-2-y1)pyrimidin-4-yl)-1′,2′- |
| | dihydrospiro[cyclopropane-1,3′-pyrrolo[3,2-c]pyridin]-6′-yl)acetamide |
| | 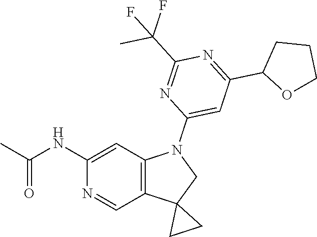 [SMILES: CC(=O)Nc1cc2c(cn1)C1(CC1)CN2c1cc(C2CCCO2)nc(C(C)(F)F)n1] |
| | SM: 6′-chloro-1′-(2-(1,1-difluoroethyl)-6-(tetrahydrofuran-2-yl)pyrimidin-4- |
| | yl)-1′,2′-dihydrospiro[cyclopropane-1,3′-pyrrolo[3,2-c]pyridine] |
| | (Preparation 145) |
| | 15 mg, LCMS m/z = 416.2 [M + H]⁺. ¹H NMR: (400 MHz, CDCl₃) δ: ppm |
| | 9.20-9.10 (m, 1H), 8.18 (br s, 1H), 7.50 (s, 1H), 6.90-6.80 (m, 1H), 5.10- |
| | 5.00 (m, 1H), 4.20-4.10 (m, 3H), 4.10-4.00 (m, 1H), 2.60-2.50 (m, 1H), |
| | 2.30-2.10 (m, 6H), 2.10-2.00 (m, 2H), 2.00-1.90 (m, 1H), 1.20 (br d, |
| | 4H, J = 4.0 Hz). |
| 27 | N-(1′-(6-methylpyrimidin-4-yl)-1′,2′-dihydrospiro[cyclopropane-1,3′- |
| | pyrrolo[3,2-c]pyridin]-6′-yl)acetamide |
| | 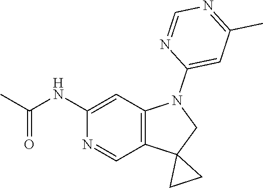 [SMILES: CC(=O)Nc1cc2c(cn1)C1(CC1)CN2c1cc(C)ncn1] |
| | SM: 6′-chloro-1′-(6-methylpyrimidin-4-yl)-1′,2′-dihydrospiro[cyclopropane- |
| | 1,3′-pyrrolo[3,2-c]pyridine] (Preparation 101) |
| | 13 mg, LCMS m/z = 296.1 [M + H]⁺. ¹H NMR: (400 MHz, CDCl₃) δ: ppm δ |
| | 9.20-9.00 (m, 1H), 8.86 (s, 1H), 8.34 (br s, 1H), 7.47 (s, 1H), 6.64 (s, 1H), |
| | 4.13 (s, 2H), 2.51 (s, 3H), 2.23 (s, 3H), 1.20-1.10 (m, 4H). |
| 28 | N-(1′-(2,5-dimethylpyrazolo[1,5-a]pyrimidin-7-yl)-1′,2′- |
| | dihydrospiro[cyclopropane-1,3′-pyrrolo[3,2-c]pyridin]-6′-yl)acetamide |
| | 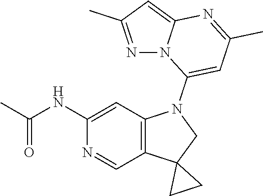 [SMILES: CC(=O)Nc1cc2c(cn1)C1(CC1)CN2c1cc(C)nc2cc(C)nn12] |
| | SM: 6′-chloro-1′-(2,5-dimethylpyrazolo[1,5-a]pyrimidin-7-yl)-1′,2′- |
| | dihydrospiro[cyclopropane-1,3′-pyrrolo[3,2-c]pyridine] (Preparation 115) |
| | 28 mg, LCMS m/z = 349.1 [M + H]⁺. ¹H NMR: (400 MHz, CDCl₃) δ: ppm |
| | 8.37 (br s, 1H), 7.96 (s, 1H), 7.51 (s, 1H), 6.79 (s, 1H), 6.36 (s, 1H), 4.55 (s, |
| | 2H), 2.59 (s, 3H), 2.49 (s, 3H), 2.19 (s, 3H), 1.19 (s, 4H). |
| 29 | N-(1′-(6-methyl-2-(methylthio)pyrimidin-4-yl)-1′,2′- |
| | dihydrospiro[cyclopropane-1,3′-pyrrolo[3,2-c]pyridin]-6′-yl)acetamide |
| | 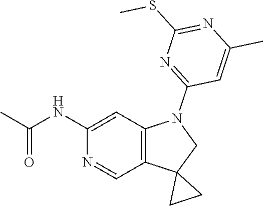 [SMILES: CSc1nc(C)cc(N2CC3(CC3)c3cnc(NC(C)=O)cc32)n1] |
| | SM: 6′-chloro-1′-(6-methyl-2-(methylthio)pyrimidin-4-yl)-1′,2′- |
| | dihydrospiro[cyclopropane-1,3′-pyrrolo[3,2-c]pyridine] (Preparation 121) |
| | 11 mg, LCMS m/z = 342.0 [M + H]⁺. ¹H NMR: (400 MHz, CDCl₃) δ: ppm |
| | 8.94 (s, 1H), 8.60-8.50 (m, 1H), 7.44 (s, 1H), 6.27 (s, 1H), 4.12 (s, 2H), 2.69 |
| | (s, 3H), 2.43 (s, 3H), 2.22 (s, 3H), 1.30-1.10 (m, 4H). |
| 30 | N-(1-(2-(1, 1-difluoroethyl)pyrimidin-4-yl)-2,3-dihydro-1H-pyrrolo[3,2- |
| | c]pyridin-6-yl)acetamide |
| | 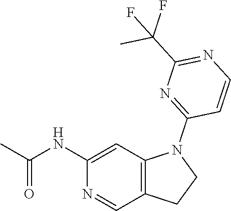 [SMILES: CC(=O)Nc1cc2c(cn1)CCN2c1ccnc(C(C)(F)F)n1] |
| | SM: 6-chloro-1-(2-(1,1-difluoroethyl)pyrimidin-4-yl)-2,3-dihydro-1H- |
| | pyrrolo[3,2-c]pyridine (Preparation 90) |
| | 12 mg, 14% yield, white powder |
| | LCMS m/z = 320.0 [M + H]⁺. ¹H NMR: (500 MHz, MeOH-d₄) δ: ppm 9.02 |
| | (s, 1H), 8.56 (d, 1H, J = 6.0 Hz), 8.04 (s, 1H), 6.97 (d, 1H, J = 6.0 Hz), 4.21 (t, |
| | 2H, J-8.5 Hz), 3.30-3.20 (m, 2H), 2.20-2.10 (m, 6H). |
| 31 | N-(1-(4-(1,1-difluoroethyl)pyrimidin-2-yl)-2,3-dihydro-1H-pyrrolo[3,2- |
| | c]pyridin-6-yl)acetamide |
| | 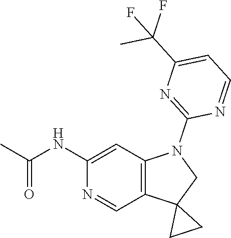 [SMILES: CC(=O)Nc1cc2c(cn1)C1(CC1)CN2c1nccc(C(C)(F)F)n1] |
| | SM: 6-chloro-1-(4-(1,1-difluoroethyl)pyrimidin-2-yl)-2,3-dihydro-1H- |
| | pyrrolo[3,2-c]pyridine (Preparation 91) |
| | 27 mg, 62% yield, white powder. |
| | LCMS m/z = 320.0 [M + H]⁺. ¹H NMR: (400 MHz, CDCl₃) δ: ppm 9.20-9.00 |
| | (m, 1H), 8.73 (d, 1H, J-5.0 Hz), 8.21 (br s, 1H), 7.96 (s, 1H), 7.17 (d, |
| | 1H, J = 5.0 Hz), 4.40-4.30 (m, 2H), 3.20 (t, 2H, J = 8.5 Hz), 2.30-2.10 (m, |
| | 6H). |
| 32 | N-(1-(6-(1,1-difluoroethyl)pyridin-2-yl)-2,3-dihydro-1H-pyrrolo[3,2- |
| | c]pyridin-6-yl)acetamide |
| | 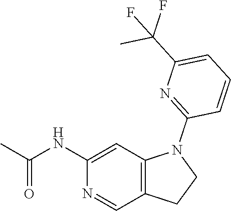 [SMILES: CC(=O)Nc1cc2c(cn1)CCN2c1cccc(C(C)(F)F)n1] |
| | SM: 6-chloro-1-(6-(1,1-difluoroethyl)pyridin-2-yl)-2,3-dihydro-1H- |
| | pyrrolo[3,2-c]pyridine (Preparation 79) |
| | 16 mg, LCMS m/z = 319.1 [M + H]⁺. ¹H NMR: (400 MHz, CDCl₃) δ: ppm |
| | 8.90-8.80 (m, 1H), 8.40-8.10 (m, 1H), 7.91 (s, 1H), 7.78 (t, 1H, J = 7.9 Hz), |
| | 7.30-7.28 (m, 1H), 7.00-6.90 (m, 1H), 4.18 (t, 2H, J = 8.7 Hz), 3.21 (t, |
| | 2H, J = 8.7 Hz), 2.30-2.10 (m, 6H). |
| 33 | N-(1-(6-(tetrahydrofuran-3-yl)pyridin-2-yl)-2,3-dihydro-1H-pyrrolo[3,2- |
| | c]pyridin-6-yl)acetamide |
| | 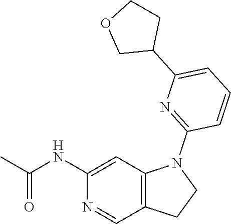 [SMILES: CC(=O)Nc1cc2c(cn1)CCN2c1cccc(C2CCOC2)n1] |
| | SM: 6-chloro-1-(6-(tetrahydrofuran-3-yl)pyridin-2-yl)-2,3-dihydro-1H- |
| | pyrrolo[3,2-c]pyridine (Preparation 80) |
| | 5 mg, LCMS m/z = 325.1 [M + H]⁺. ¹H NMR: (400 MHz, MeOH-d₄) δ: ppm |
| | 8.78 (s, 1H), 7.89 (s, 1H), 7.68 (dd, 1H, J = 7.5, 8.3 Hz), 6.92 (d, 1H, J = 7.5 |
| | Hz), 6.80 (d, 1H, J = 8.3 Hz), 4.30-4.10 (m, 4H), 4.00-3.90 (m, 2H), 3.61 |
| | (quin, 1H, J = 7.9 Hz), 3.30-3.20 (m, 2H), 2.50-2.30 (m, 2H), 2.17 (s, 3H). |
| 34 | N-(1-(2-(1,1-difluoroethyl)-6-methoxypyrimidin-4-yl)-2,3-dihydro-1H- |
| | pyrrolo[3,2-c]pyridin-6-yl)acetamide |
| | 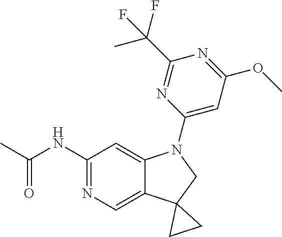 [SMILES: COc1cc(N2CC3(CC3)c3cnc(NC(C)=O)cc32)nc(C(C)(F)F)n1] |
| | SM: 6-chloro-1-(2-(1,1-difluoroethyl)-6-methoxypyrimidin-4-y1)-2,3- |
| | dihydro-1H-pyrrolo[3,2-c]pyridine (Preparation 148) |
| | 10 mg, LCMS m/z = 350.1 [M + H]⁺. ¹H NMR: (400 MHz, CDCl₃) δ: ppm |
| | 9.30-8.90 (m, 1H), 8.50-8.20 (m, 1H), 7.94 (s, 1H), 6.06 (s, 1H), 4.11 (t, |
| | 2H, J = 8.7 Hz), 4.05 (s, 3H), 3.24 (t, 2H, J = 8.4 Hz), 2.20-2.10 (m, 6H). |
| 35 | N-(1-(2-(1,1-difluoroethyl)-6-(methylamino)pyrimidin-4-yl)-2,3-dihydro- |
| | 1H-pyrrolo[3,2-c]pyridin-6-yl)acetamide |
| | 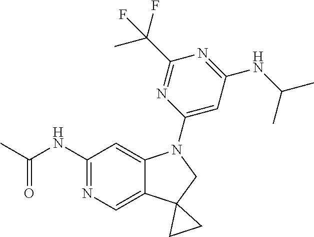 [SMILES: CC(=O)Nc1cc2c(cn1)C1(CC1)CN2c1cc(NC(C)C)nc(C(C)(F)F)n1] |
| | SM: 6-(6-chloro-2,3-dihydro-1H-pyrrolo[3,2-c]pyridin-1-yl)-2-(1,1- |
| | difluoroethyl)-N-methylpyrimidin-4-amine (Preparation 149) |
| | 12 mg, LCMS m/z = 349.2 [M + H]⁺. ¹H NMR: (400 MHz, MeOH-d₄) δ: |
| | ppm 8.40-8.20 (m, 1H), 7.90 (s, 1H), 6.00-5.00 (m, 1H), 4.40-4.20 (m, 2H), |
| | 3.33 (br d, 2H, J=1.5 Hz), 2.96 (s, 3H), 2.27 (s, 3H), 2.01 (t, 3H, J=18.6 |
| | Hz). |
| 36 | N-(1′-(4-(1, 1-difluoroethyl)pyrimidin-2-yl)-2-methyl-1′,2′- |
| | dihydrospiro[cyclopropane-1,3′-pyrrolo[3,2-c]pyridin]-6′-yl)acetamide |
| | 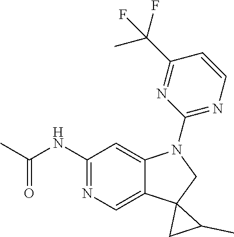 [SMILES: CC(=O)Nc1cc2c(cn1)C1(CC1C)CN2c1nccc(C(C)(F)F)n1] |
| | SM: 6′-chloro-1′-(4-(1,1-difluoroethyl)pyrimidin-2-yl)-2-methyl-1′,2′- |
| | dihydrospiro[cyclopropane-1,3′-pyrrolo[3,2-c]pyridine] (Preparation 94) |
| | 13 mg, LCMS m/z = 360.2 [M + H]⁺. ¹H NMR: (400 MHz, CDCl₃) δ: ppm |
| | 9.13 (br s, 1H), 8.80-8.70 (m, 1H), 8.31 (br s, 1H), 7.60 (s, 1H), 7.17 (d, |
| | 1H, J = 5.0 Hz), 4.30-4.20 (m, 2H), 2.30-2.10 (m, 6H), 1.40-1.30 (m, 2H), |
| | 1.17 (d, 3H, J = 6.0 Hz), 0.92-0.89 (m, 1H). |
| 37 | N-(1′-(2-(1, 1-difluoroethyl)pyrimidin-4-yl)-2-methyl-1′,2′- |
| | dihydrospiro[cyclopropane-1,3′-pyrrolo[3,2-c]pyridin]-6′-yl)acetamide |
| | 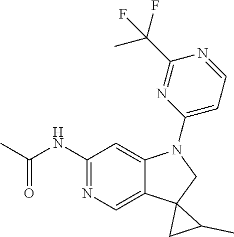 [SMILES: CC(=O)Nc1cc2c(cn1)C1(CC1C)CN2c1ccnc(C(C)(F)F)n1] |
| | SM: 6′-chloro-1′-(2-(1,1-difluoroethyl)pyrimidin-4-yl)-2-methyl-1′,2′- |
| | dihydrospiro[cyclopropane-1,3′-pyrrolo[3,2-c]pyridine] (Preparation 95) |
| | 11 mg, LCMS m/z = 360.2 [M + H]⁺. ¹H NMR: (400 MHz, CDCl₃) δ: ppm |
| | 9.08 (br s, 1H), 8.70-8.60 (m, 1H), 8.25 (br s, 1H), 7.64 (s, 1H), 6.75 (br d, |
| | 1H, J-6.0 Hz), 4.20 (d, 1H, J = 10.0 Hz), 3.99 (d, 1H, J = 9.8 Hz), 2.30-2.10 |
| | (m, 6H), 1.40-1.30 (m, 2H), 1.18 (d, 3H, J = 6.0 Hz), 0.95 (t, 1H, J = 5.4 Hz). |
| 38 | N-(1-(2-(1,1-difluoroethyl)pyrimidin-4-yl)-3,3-dimethyl-2,3-dihydro-1H- |
| | pyrrolo[3,2-c]pyridin-6-yl)acetamide |
| | 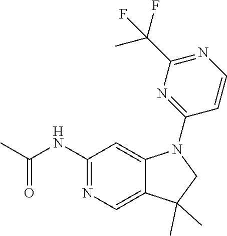 [SMILES: CC(=O)Nc1cc2c(cn1)C(C)(C)CN2c1ccnc(C(C)(F)F)n1] |
| | SM: 6-chloro-1-(2-(1,1-difluoroethyl)pyrimidin-4-yl)-3,3-dimethyl-2,3- |
| | dihydro-1H-pyrrolo[3,2-c]pyridine (Preparation 92) |
| | 31 mg, 48% yield, white powder. |
| | LCMS m/z = 348.1 [M + H]⁺. ¹H NMR: (400 MHz, CDCl₃) δ: ppm 9.14 (br |
| | s, 1H), 9.10-8.90 (m, 1H), 8.67 (d, 1H, J = 5.8 Hz), 7.90 (s, 1H), 6.82 (d, |
| | 1H, J = 6.0 Hz), 3.94 (s, 2H), 2.30-2.20 (m, 6H), 1.48 (s, 6H). |
| 39 | N-(1-(4-(1, 1-difluoroethyl)pyrimidin-2-yl)-3,3-dimethyl-2,3-dihydro-1H- |
| | pyrrolo[3,2-c]pyridin-6-yl)acetamide |
| | 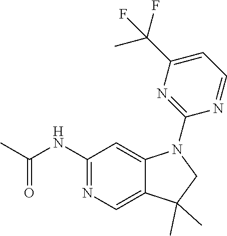 [SMILES: CC(=O)Nc1cc2c(cn1)C(C)(C)CN2c1nccc(C(C)(F)F)n1] |
| | SM: 6-chloro-1-(4-(1,1-difluoroethyl)pyrimidin-2-yl)-3,3-dimethyl-2,3- |
| | dihydro-1H-pyrrolo[3,2-c]pyridine (Preparation 93) |
| | 21 mg, LCMS m/z = 348.1 [M + H]⁺. ¹H NMR: (400 MHz, CDCl₃) δ: ppm |
| | 9.05 (s, 1H), 8.72 (d, 1H, J = 5.0 Hz), 8.30-8.10 (m, 1H), 7.93 (s, 1H), 7.20- |
| | 7.10 (m, 1H), 4.09 (s, 2H), 2.20-2.10 (m, 6H), 1.44 (s, 6H). |
| 40 | N-(1′-(4-(1,1-difluoroethyl)pyrimidin-2-yl)-1′,2′-dihydrospiro[cyclobutane- |
| | 1,3′-pyrrolo[3,2-c]pyridin]-6′-yl)acetamide |
| | 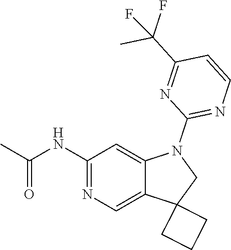 [SMILES: CC(=O)Nc1cc2c(cn1)C1(CCC1)CN2c1nccc(C(C)(F)F)n1] |
| | SM: 6′-chloro-1′-(4-(1,1-difluoroethyl)pyrimidin-2-yl)-1′,2′- |
| | dihydrospiro[cyclobutane-1,3′-pyrrolo[3,2-c]pyridine] (Preparation 96) |
| | 32 mg, LCMS m/z = 360.2 [M + H]⁺. ¹H NMR: (400 MHz, CDCl₃) δ: ppm |
| | 9.10-8.90 (m, 1H), 8.72 (d, 1H, J = 5.0 Hz), 8.40-8.20 (m, 1H), 8.22 (s, 1H), |
| | 7.20-7.10 (m, 1H), 4.40 (s, 2H), 2.50-2.40 (m, 2H), 2.40-2.30 (m, 2H), 2.30- |
| | 2.10 (m, 8H). |
| 41 | N-(1′-(2-(1, 1-difluoroethyl)pyrimidin-4-yl)-1′,2′-dihydrospiro[cyclobutane- |
| | 1,3′-pyrrolo[3,2-c]pyridin]-6′-yl)acetamide |
| | 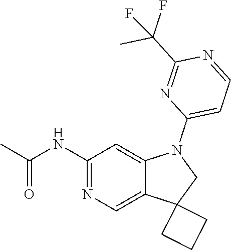 [SMILES: CC(=O)Nc1cc2c(cn1)C1(CCC1)CN2c1ccnc(C(C)(F)F)n1] |
| | SM: 6′-chloro-1′-(2-(1,1-difluoroethyl)pyrimidin-4-yl)-1′,2′- |
| | dihydrospiro[cyclobutane-1,3′-pyrrolo[3,2-c]pyridine] (Preparation 97)30 |
| | mg, LCMS m/z = 360.2 [M + H]⁺. ¹H NMR: (400 MHz, CDCl₃) δ: ppm ¹H |
| | NMR (CDCl₃, 400 MHz) δ 8.99 (br s, 1H), 8.62 (d, 1H, J = 6.0 Hz), 8.26 (s, |
| | 1H), 8.20-8.00 (m, 1H), 6.90-6.70 (m, 1H), 4.21 (s, 2H), 2.60-2.50 (m, 2H), |
| | 2.39 (ddd, 2H, J = 6.0, 8.8, 12.2 Hz), 2.30-2.10 (m, 8H). |
| 42 | N-(1′-(6-(1, 1-difluoroethyl)pyridin-2-yl)-1′,2′-dihydrospiro[cyclobutane- |
| | 1,3′-pyrrolo[3,2-c]pyridin]-6′-yl)acetamide |
| | 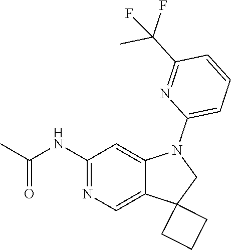 [SMILES: CC(=O)Nc1cc2c(cn1)C1(CCC1)CN2c1cccc(C(C)(F)F)n1] |
| | SM: 6′-chloro-1′-(6-(1,1-difluoroethyl)pyridin-2-yl)-1′,2′- |
| | dihydrospiro[cyclobutane-1,3′-pyrrolo[3,2-c]pyridine] (Preparation 77) |
| | 30 mg, LCMS m/z = 359.2 [M + H]⁺. ¹H NMR: (400 MHz, CDCl₃) δ: ppm |
| | 8.81 (s, 1H), 8.30-8.10 (m, 2H), 7.78 (t, 1H, J = 7.9 Hz), 7.29 (s, 1H), 7.00- |
| | 6.90 (m, 1H), 4.20 (s, 2H), 2.50-2.40 (m, 2H), 2.36 (ddd, 2H, J = 6.1, 8.8, |
| | 12.2 Hz), 2.30-2.10 (m, 6H), 2.10-2.00 (m, 2H). |
| 43 | N-(1′-(4-(1,1-difluoroethyl)pyrimidin-2-yl)-1′,2′-dihydrospiro[cyclopentane- |
| | 1,3′-pyrrolo[3,2-c]pyridin]-6′-yl)acetamide |
| | 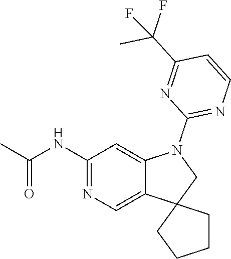 [SMILES: CC(=O)Nc1cc2c(cn1)C1(CCCC1)CN2c1nccc(C(C)(F)F)n1] |
| | SM: 6′-chloro-1′-(4-(1,1-difluoroethyl)pyrimidin-2-yl)-1′,2′- |
| | dihydrospiro[cyclopentane-1,3′-pyrrolo[3,2-c]pyridine] (Preparation 98) |
| | 25 mg, LCMS m/z = 374.2 [M + H]⁺. ¹H NMR: (400 MHz, CDCl₃) δ: ppm |
| | 9.05 (br s, 1H), 8.71 (d, 1H, J = 5.0 Hz), 8.26 (br s, 1H), 7.94 (s, 1H), 7.16 (d, |
| | 1H, J = 5.0 Hz), 4.13 (s, 2H), 2.30-2.10 (m, 6H), 2.00-1.70 (m, 10H). |
| 44 | N-(1′-(2-(1, 1-difluoroethyl)pyrimidin-4-yl)-1′,2′-dihydrospiro[cyclopentane- |
| | 1,3′-pyrrolo[3,2-c]pyridin]-6′-yl)acetamide |
| | 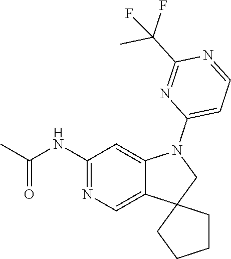 [SMILES: CC(=O)Nc1cc2c(cn1)C1(CCCC1)CN2c1ccnc(C(C)(F)F)n1] |
| | SM: 6′-chloro-1′-(2-(1,1-difluoroethyl)pyrimidin-4-yl)-1′,2′- |
| | dihydrospiro[cyclopentane-1,3′-pyrrolo[3,2-c]pyridine] (Preparation 67)20 |
| | mg, LCMS m/z = 374.2 [M + H]⁺. ¹H NMR: (400 MHz, CDCl₃) δ: ppm 9.10- |
| | 8.90 (m, 1H), 8.61 (d, 1H, J = 6.0 Hz), 8.22 (br s, 1H), 7.98 (s, 1H), 6.78 (d, |
| | 1H, J = 5.8 Hz), 3.94 (s, 2H), 2.30-2.20 (m, 6H), 2.00-1.90 (m, 6H), 1.90- |
| | 1.80 (m, 4H). |
| 45 | N-(1′-(6-(1,1-difluoroethyl)pyridin-2-yl)-1′,2′-dihydrospiro[cyclopentane- |
| | 1,3′-pyrrolo[3,2-c]pyridin]-6′-yl)acetamide |
| | 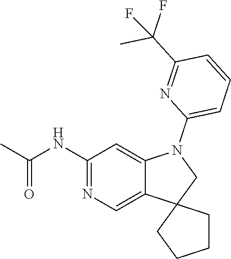 [SMILES: CC(=O)Nc1cc2c(cn1)C1(CCCC1)CN2c1cccc(C(C)(F)F)n1] |
| | SM: 6′-chloro-1′-(6-(1,1-difluoroethyl)pyridin-2-yl)-1′,2′- |
| | dihydrospiro[cyclopentane-1,3′-pyrrolo[3,2-c]pyridine] (Preparation 82)41 |
| | mg, LCMS m/z = 373.2 [M + H]⁺. ¹H NMR: (400 MHz, CDCl₃) δ: ppm 8.84 |
| | (s, 1H), 8.16 (br s, 1H), 7.89 (s, 1H), 7.77 (t, 1H, J = 7.9 Hz), 7.30-7.29 (m, |
| | 1H), 7.00-6.90 (m, 1H), 3.93 (s, 2H), 2.30-2.10 (m, 6H), 2.00-1.90 (m, 6H), |
| | 1.90-1.70 (m, 4H). |
| 46 | N-(1′-(4-(1, 1-difluoroethyl)pyrimidin-2-yl)-1′,2,2′,3,5,6- |
| | hexahydrospiro[pyran-4,3′-pyrrolo[3,2-c]pyridin]-6′-yl)acetamide |
| | 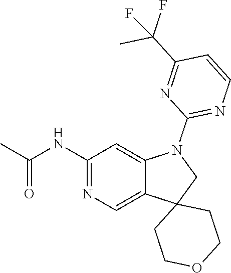 [SMILES: CC(=O)Nc1cc2c(cn1)C1(CCOCC1)CN2c1nccc(C(C)(F)F)n1] |
| | SM: 6′-chloro-1′-(4-(1,1-difluoroethyl)pyrimidin-2-yl)- |
| | 1′,2,2′,3,5,6-hexahydrospiro[pyran-4,3′-pyrrolo[3,2-c]pyridine] (Preparation |
| | 66)45 mg, LCMS m/z = 390.2 [M + H]⁺. ¹H NMR: (400 MHz, CDCl₃) δ: |
| | ppm 9.08 (s, 1H), 8.73 (d, 1H, J = 5.0 Hz), 8.30-8.20 (m, 1H), 8.00 (s, 1H), |
| | 7.19 (d, 1H, J = 5.0 Hz), 4.28 (s, 2H), 4.04 (td, 2H, J = 2.3, 10.0 Hz), 3.67 (dt, |
| | 2H, J = 1.9, 12.1 Hz), 2.30-2.10 (m, 6H), 2.10-2.00 (m, 2H), 1.80-1.70 (m, |
| | 2H). |
| 47 | N-(1′-(2-(1, 1-difluoroethyl)pyrimidin-4-yl)-1′,2,2′,3,5,6- |
| | hexahydrospiro[pyran-4,3′-pyrrolo[3,2-c]pyridin]-6′-yl)acetamide |
| | 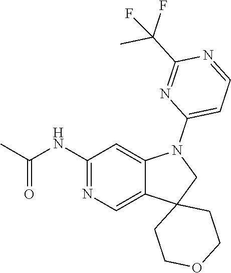 [SMILES: CC(=O)Nc1cc2c(cn1)C1(CCOCC1)CN2c1ccnc(C(C)(F)F)n1] |
| | SM: 6′-chloro-1′-(2-(1,1-difluoroethyl)pyrimidin-4-yl)-1′,2,2′,3,5,6- |
| | hexahydrospiro[pyran-4,3′-pyrrolo[3,2-c]pyridine] (Preparation 99)5 mg, |
| | LCMS m/z = 390.2 [M + H]⁺. ¹H NMR: (400 MHz, CDCl₃) δ: ppm 9.09 (s, |
| | 1H), 8.67 (d, 1H, J = 5.8 Hz), 8.01 (s, 1H), 6.90 (d, 1H, J = 5.8 Hz), 4.10-4.00 |
| | (m, 4H), 3.63 (dt, 2H, J = 1.8, 12.0 Hz), 2.30-2.20 (m, 5H), 2.10-2.00 (m, |
| | 2H), 1.80-1.70 (m, 2H). |
| 48 | N-(1′-(6-(1,1-difluoroethyl)pyridin-2-yl)-1′,2,2′,3,5,6-hexahydrospiro[pyran- |
| | 4,3′-pyrrolo[3,2-c]pyridin]-6′-yl)acetamide |
| | 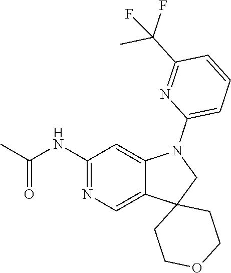 [SMILES: CC(=O)Nc1cc2c(cn1)C1(CCOCC1)CN2c1cccc(C(C)(F)F)n1] |
| | SM: 6′-chloro-1′-(6-(1,1-difluoroethyl)pyridin-2-yl)-1′,2,2′,3,5,6- |
| | hexahydrospiro[pyran-4,3′-pyrrolo[3,2-c]pyridine] (Preparation 83)44 mg, |
| | LCMS m/z = 389.2 [M + H]⁺. ¹H NMR: (400 MHz, CDCl₃) δ: ppm δ 8.84 (s, |
| | 1H), 8.35 (br s, 1H), 7.95 (s, 1H), 7.81 (t, 1H, J = 8.0 Hz), 7.31 (d, 1H, J = 7.5 |
| | Hz), 7.02-7.00 (m, 1H), 4.10-4.00 (m, 4H), 3.63 (dt, 2H, J = 2.0, 12.0 Hz), |
| | 2.30-2.10 (m, 6H), 2.10-2.00 (m, 2H), 1.73 (dd, 2H, J = 1.6, 13.7 Hz). |
| 49 | N-(1′-(2-(1,1-difluoroethyl)-6-((2-methoxyethyl)amino)pyrimidin-4-yl)- |
| | 1′,2′-dihydrospiro[cyclopropane-1,3'-pyrrolo[3,2-c]pyridin]-6'-yl)acetamide |
| | 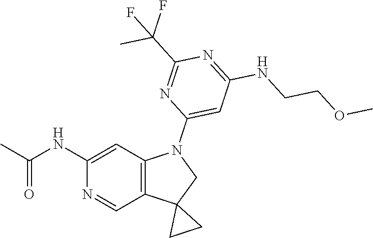 [SMILES: COCCNc1cc(N2CC3(CC3)c3cnc(NC(C)=O)cc32)nc(C(C)(F)F)n1] |
| | SM: 6-(6′-chlorospiro[cyclopropane-1,3′-pyrrolo[3,2-c]pyridin]-1′(2′H)-yl)- |
| | 2-(1,1-difluoroethyl)-N-(2-methoxyethyl)pyrimidin-4-amine (Preparation |
| | 132). |
| | LCMS m/z = 419.3 [M + H]⁺; ¹H NMR (400 MHz, MeOH-d₄) δ 9.00-8.70 |
| | (m, 1H), 7.51 (s, 1H), 5.77 (s, 1H), 4.05 (s, 2H), 3.60-3.50 (m, 4H), 3.38 (s, |
| | 3H), 2.16 (s, 3H), 2.06 (t, 3H, J = 18.7 Hz), 1.18 (s, 4H) |
| 50 | N-(1′-(2-(1, 1-difluoroethyl)-6-((2-methoxyethyl)(methyl)amino)pyrimidin- |
| | 4-yl)-1′,2′-dihydrospiro[cyclopropane-1,3′-pyrrolo[3,2-c]pyridin]-6′- |
| | yl)acetamide |
| | 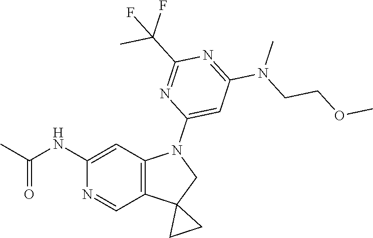 [SMILES: COCCN(C)c1cc(N2CC3(CC3)c3cnc(NC(C)=O)cc32)nc(C(C)(F)F)n1] |
| | SM: 6-(6′-chlorospiro[cyclopropane-1,3′-pyrrolo[3,2-c]pyridin]-1′(2′H)-yl)- |
| | 2-(1,1-difluoroethyl)-N-(2-methoxyethyl)-N-methylpyrimidin-4-amine |
| | (Preparation 133). |
| | LCMS m/z = 433.2 [M + H]⁺; ¹H NMR (400 MHz, MeOH-d₄) δ 8.86 (s, 1H), |
| | 7.53 (s, 1H), 5.81 (s, 1H), 4.12 (s, 2H), 3.90-3.80 (m, 2H), 3.62 (t, 2H, J = 5.5 |
| | Hz), 3.36 (s, 3H), 3.15 (s, 3H), 2.16 (s, 3H), 2.04 (t, 3H, J = 18.7 Hz), 1.23- |
| | 1.17 (m, 4H). |
| 51 | N-(1′-(6-(azetidin-1-yl)-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | 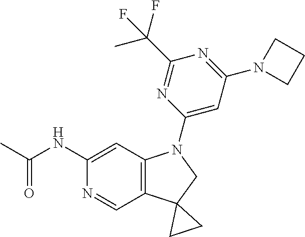 [SMILES: CC(=O)Nc1cc2c(cn1)C1(CC1)CN2c1cc(N2CCC2)nc(C(C)(F)F)n1] |
| | SM: 1′-(6-(azetidin-1-yl)-2-(1,1-difluoroethyl)pyrimidin-4-yl)-6′-chloro- |
| | 1′,2′-dihydrospiro[cyclopropane-1,3′-pyrrolo[3,2-c]pyridine (Preparation |
| | 134) |
| | LCMS m/z = 401.2 [M + H]⁺ ¹H NMR (400 MHz, CDCl₃) δ: 8.88 (br s, 1H), |
| | 8.09 (br s, 1H), 7.43 (s, 1H), 5.45 (s, 1H), 4.20-4.10 (m, 4H), 4.07 (s, 2H), |
| | 2.50-2.40 (m, 2H), 2.20 (s, 3H), 2.12 (t, 3H, J = 18.8 Hz), 1.20-1.10 (m, 4H). |
| 52 | N-(1′-(2-(1,1-difluoroethyl)-6-morpholinopyrimidin-4-y1)-1′,2′- |
| | dihydrospiro[cyclopropane-1,3′-pyrrolo[3,2-c]pyridin]-6′-yl)acetamide |
| | 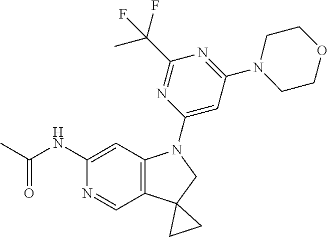 [SMILES: CC(=O)Nc1cc2c(cn1)C1(CC1)CN2c1cc(N2CCOCC2)nc(C(C)(F)F)n1] |
| | SM: 4-(6-(6′-chlorospiro[cyclopropane-1,3′-pyrrolo[3,2-c]pyridin]-1′(2′H)- |
| | yl)-2-(1,1-difluoroethyl)pyrimidin-4-yl)morpholine (Preparation 135). |
| | LCMS m/z = 431.2 [M + H]⁺ ¹H NMR (400 MHz, CDCl₃) δ 9.00-8.70 (m, |
| | 1H), 8.17 (br s, 1H), 7.45 (s, 1H), 5.86 (s, 1H), 4.11 (s, 2H), 3.90-3.80 (m, |
| | 4H), 3.80-3.60 (m, 4H), 2.20 (s, 3H), 2.20-2.10 (m, 3H), 1.20-1.10 (m, 4H). |
| 53 | N-(1′-(2-(1, 1-difluoroethyl)-6-isopropylpyrimidin-4-yl)-1′,2′- |
| | dihydrospiro[cyclopropane-1,3′-pyrrolo[3,2-c]pyridin]-6′-yl)acetamide |
| | 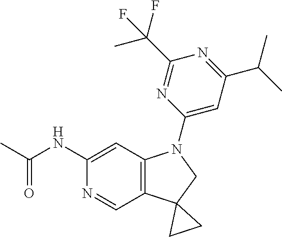 [SMILES: CC(=O)Nc1cc2c(cn1)C1(CC1)CN2c1cc(C(C)C)nc(C(C)(F)F)n1] |
| | SM: 6′-chloro-1′-(2-(1,1-difluoroethyl)-6-isopropylpyrimidin-4-yl)-1′,2′- |
| | dihydrospiro[cyclopropane-1,3′-pyrrolo[3,2-c]pyridine] (Preparation 138). |
| | LCMS m/z = 388.2 [M + H]⁺ ¹H NMR (400 MHz, CDCl₃) δ 9.20-8.90 (m, |
| | 1H), 8.15 (br s, 1H), 7.51 (s, 1H), 6.62 (s, 1H), 4.15 (s, 2H), 3.06 (td, |
| | 1H, J = 6.8, 13.9 Hz), 2.30-2.10 (m, 6H), 1.34 (d, 6H, J = 7.0 Hz), 1.20-1.10 |
| | (m, 4H). |
| 54 | N-(1′-(2-cyclopropyl-6-methylpyrimidin-4-yl)-1′,2′- |
| | dihydrospiro[cyclopropane-1,3′-pyrrolo[3,2-c]pyridin]-6′-yl)acetamide |
| | 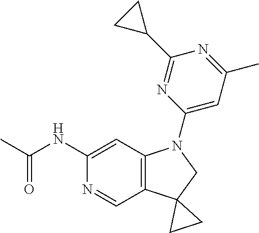 [SMILES: CC(=O)Nc1cc2c(cn1)C1(CC1)CN2c1cc(C)nc(C2CC2)n1] |
| | SM: 6′-chloro-1′-(2-cyclopropyl-6-methylpyrimidin-4-yl)-1′,2′- |
| | dihydrospiro[cyclopropane-1,3′-pyrrolo[3,2-c]pyridine] (Preparation 103). |
| | LCMS m/z = 336.2 [M + H]⁺ ¹H NMR (400 MHz, CDCl₃) δ 8.96 (br s, 1H), |
| | 8.19 (br s, 1H), 7.45 (s, 1H), 6.30 (s, 1H), 4.06 (s, 2H), 2.44 (s, 3H), 2.30- |
| | 2.20 (m, 4H), 1.40-1.30 (m, 2H), 1.20-1.10 (m, 6H). |
| 55 | N-(1′-(2-(tert-butyl)-6-methylpyrimidin-4-yl)-1′,2′- |
| | dihydrospiro[cyclopropane-1,3′-pyrrolo[3,2-c]pyridin]-6′-yl)acetamide |
| | 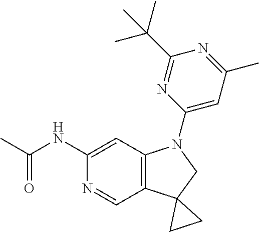 [SMILES: CC(=O)Nc1cc2c(cn1)C1(CC1)CN2c1cc(C)nc(C(C)(C)C)n1] |
| | SM: 1′-(2-(tert-butyl)-6-methylpyrimidin-4-yl)-6′-chloro-1′,2′- |
| | dihydrospiro[cyclopropane-1,3′-pyrrolo[3,2-c]pyridine] (Preparation 102) |
| | LCMS m/z = 352.2 [M + H]⁺ ¹H NMR (400 MHz, CDCl₃) δ 9.01 (br s, 1H), |
| | 8.50-8.30 (m, 1H), 7.47 (s, 1H), 6.34 (s, 1H), 4.08 (s, 2H), 2.47 (s, 3H), 2.20 |
| | (s, 3H), 1.48 (s, 9H), 1.20-1.10 (m, 4H). |
| 56 | N-(1′-(2-methoxy-6-methylpyrimidin-4-yl)-1′,2′-dihydrospiro[cyclopropane- |
| | 1,3′-pyrrolo[3,2-c]pyridin]-6′-yl)acetamide |
| | 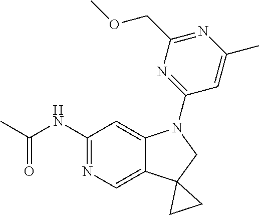 [SMILES: COCc1nc(C)cc(N2CC3(CC3)c3cnc(NC(C)=O)cc32)n1] |
| | SM: 6′-chloro-1′-(2-methoxy-6-methylpyrimidin-4-yl)-1′,2′- |
| | dihydrospiro[cyclopropane-1,3′-pyrrolo[3,2-c]pyridine] (Preparation 104). |
| | LCMS m/z = 326.1 [M + H]⁺ ¹H NMR (400 MHz, CDCl₃) δ 9.08 (s, 1H), |
| | 8.02 (br s, 1H), 7.48 (s, 1H), 6.15 (s, 1H), 4.18 (s, 3H), 4.08 (s, 2H), 2.42 (s, |
| | 3H), 2.19 (s, 3H), 1.20-1.10 (m, 4H). |
| 57 | N-(1′-(2-(methoxymethyl)-6-methylpyrimidin-4-yl)-1′,2′- |
| | dihydrospiro[cyclopropane-1,3′-pyrrolo[3,2-c]pyridin]-6′-yl)acetamide |
| | 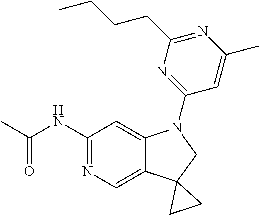 [SMILES: CCCCc1nc(C)cc(N2CC3(CC3)c3cnc(NC(C)=O)cc32)n1] |
| | SM: 6′-chloro-1′-(2-(methoxymethyl)-6-methylpyrimidin-4-yl)-1′,2′- |
| | dihydrospiro[cyclopropane-1,3′-pyrrolo[3,2-c]pyridine] (Preparation 105). |
| | LCMS m/z = 340.1 [M + H]⁺ ¹H NMR (400 MHz, CDCl₃) δ 9.14 (br s, 1H), |
| | 8.60-8.40 (m, 1H), 7.45 (s, 1H), 6.50 (s, 1H), 4.71 (s, 2H), 4.13 (s, 2H), |
| | 3.70-3.60 (m, 3H), 2.54 (s, 3H), 2.24 (s, 3H), 1.20-1.10 (m, 4H). |
| 58 | N-(1′-(2-butyl-6-methylpyrimidin-4-yl)-1′,2′-dihydrospiro[cyclopropane- |
| | 1,3′-pyrrolo[3,2-c]pyridin]-6′-yl)acetamide |
| | 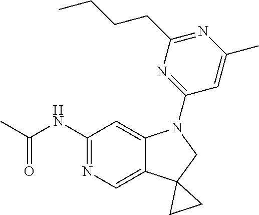 [SMILES: CCCCc1nc(C)cc(N2CC3(CC3)c3cnc(NC(C)=O)cc32)n1] |
| | SM: 1′-(2-butyl-6-methylpyrimidin-4-yl)-6′-chloro-1′,2′- |
| | dihydrospiro[cyclopropane-1,3′-pyrrolo[3,2-c]pyridine](Preparation 106). |
| | LCMS m/z = 352.2 [M + H]⁺ ¹H NMR (400 MHz, CDCl₃) δ 9.13 (br s, 1H), |
| | 8.50-8.30 (m, 1H), 7.44 (s, 1H), 6.42 (s, 1H), 4.11 (s, 2H), 3.00-2.90 (m, |
| | 2H), 2.48 (s, 3H), 2.23 (s, 3H), 2.00-1.80 (m, 2H), 1.46 (qd, 2H, J = 7.4, 14.9 |
| | Hz), 1.20-1.10 (m, 4H), 0.97 (t, 3H, J = 7.5 Hz). |
| 59 | N-(1′-(2-(ethoxymethyl)-6-methylpyrimidin-4-yl)-1′,2′- |
| | dihydrospiro[cyclopropane-1,3′-pyrrolo[3,2-c]pyridin]-6′-yl)acetamide |
| | 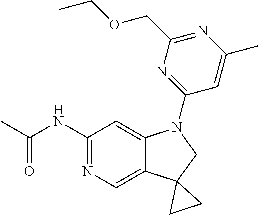 [SMILES: CCOCc1nc(C)cc(N2CC3(CC3)c3cnc(NC(C)=O)cc32)n1] |
| | SM; 6′-chloro-1′-(2-(ethoxymethyl)-6-methylpyrimidin-4-yl)-1′,2′- |
| | dihydrospiro[cyclopropane-1,3′-pyrrolo[3,2-c]pyridine] (Preparation 107). |
| | LCMS m/z = 354.1 [M + H]⁺; ¹H NMR (400 MHz, CDCl₃) δ 9.05 (br s, 1H), |
| | 8.30-8.20 (m, 1H), 7.47 (s, 1H), 6.52 (s, 1H), 4.73 (s, 2H), 4.13 (s, 2H), 3.76 |
| | (q, 2H, J = 7.0 Hz), 2.53 (s, 3H), 2.23 (s, 3H), 1.32 (t, 3H, J = 7.0 Hz), 1.20- |
| | 1.10 (m, 4H). |
| 60 | N-(1′-(2-cyclobutyl-6-methylpyrimidin-4-yl)-1′,2′- |
| | dihydrospiro[cyclopropane-1,3′-pyrrolo[3,2-c]pyridin]-6′-yl)acetamide |
| | 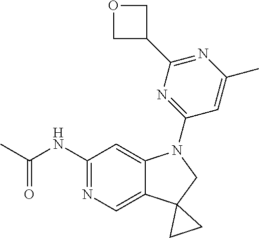 [SMILES: CC(=O)Nc1cc2c(cn1)C1(CC1)CN2c1cc(C)nc(C2COC2)n1] |
| | SM: 6′-chloro-1′-(2-cyclobutyl-6-methylpyrimidin-4-yl)-1′,2′- |
| | dihydrospiro[cyclopropane-1,3′-pyrrolo[3,2-c]pyridine] (Preparation 108). |
| | LCMS m/z = 350.1 [M + H]⁺; ¹H NMR (400 MHz, CDCl₃) δ 9.20-9.10 (m, |
| | 1H), 8.41 (br s, 1H), 7.45 (s, 1H), 6.38 (s, 1H), 4.12 (s, 2H), 3.79 (quin, |
| | 1H, J = 8.8 Hz), 2.70-2.50 (m, 2H), 2.50-2.40 (m, 5H), 2.30-2.20 (m, 3H), |
| | 2.20-2.00 (m, 2H), 1.20-1.10 (m, 4H). |
| 61 | N-(1′-(6-methyl-2-(trifluoromethyl)pyrimidin-4-yl)-1′,2′- |
| | dihydrospiro[cyclopropane-1,3′-pyrrolo[3,2-c]pyridin]-6′-yl)acetamide |
| | 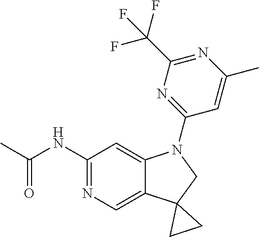 [SMILES: CC(=O)Nc1cc2c(cn1)C1(CC1)CN2c1cc(C)nc(C(F)(F)F)n1] |
| | SM: 6′-chloro-1′-(6-methyl-2-(trifluoromethyl)pyrimidin-4-yl)-1′,2′- |
| | dihydrospiro[cyclopropane-1,3′-pyrrolo[3,2-c]pyridine] (Preparation 109). |
| | LCMS m/z = 364.1 [M + H]⁺; ¹H NMR (400 MHz, CDCl₃) δ 9.15 (br s, 1H), |
| | 9.00-8.70 (m, 1H), 7.47 (s, 1H), 6.78 (s, 1H), 4.20 (s, 2H), 2.62 (s, 3H), 2.27 |
| | (s, 3H), 1.24 (s, 4H). |
| 62 | N-(1′-(2-(cyclopropylmethyl)-6-methylpyrimidin-4-yl)-1′,2′- |
| | dihydrospiro[cyclopropane-1,3′-pyrrolo[3,2-c]pyridin]-6′-yl)acetamide |
| | 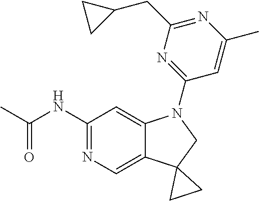 [SMILES: CC(=O)Nc1cc2c(cn1)C1(CC1)CN2c1cc(C)nc(CC2CC2)n1] |
| | SM: 6′-chloro-1′-(2-(cyclopropylmethyl)-6-methylpyrimidin-4-yl)-1′,2′- |
| | dihydrospiro[cyclopropane-1,3′-pyrrolo[3,2-c]pyridine] (Preparation 110). |
| | LCMS m/z = 350.2 [M + H]⁺; ¹H NMR (400 MHz, CDCl₃) δ 9.30-9.00 (m, |
| | 1H), 8.16 (br s, 1H), 7.46 (s, 1H), 6.41 (s, 1H), 4.10 (s, 2H), 2.83 (d, |
| | 2H, J = 7.0 Hz), 2.48 (s, 3H), 2.21 (s, 3H), 1.50-1.40 (m, 1H), 1.20-1.10 (m, |
| | 4H), 0.60-0.50 (m, 2H), 0.40-0.30 (m, 2H). |
| 63 | N-(1′-(2-(difluoromethyl)-6-methylpyrimidin-4-yl)-1′,2′- |
| | dihydrospiro[cyclopropane-1,3′-pyrrolo[3,2-c]pyridin]-6′-yl)acetamide |
| | 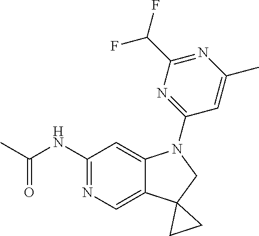 [SMILES: CC(=O)Nc1cc2c(cn1)C1(CC1)CN2c1cc(C)nc(C(F)F)n1] |
| | SM: 6′-chloro-1′-(2-(difluoromethyl)-6-methylpyrimidin-4-yl)-1′,2′- |
| | dihydrospiro[cyclopropane-1,3′-pyrrolo[3,2-c]pyridine] (Preparation 114). |
| | LCMS m/z = 346.1 [M + H]⁺; ¹H NMR (400 MHz, CDCl₃) δ 9.29 (br s, 1H), |
| | 7.50-7.40 (m, 1H), 6.90-6.50 (m, 3H), 4.21 (s, 2H), 2.70-2.50 (m, 3H), 2.30 |
| | (s, 3H), 1.30-1.20 (m, 4H). |
| 64 | N-(1′-(2-ethyl-6-methylpyrimidin-4-yl)-1′,2′-dihydrospiro[cyclopropane- |
| | 1,3′-pyrrolo[3,2-c]pyridin]-6′-yl)acetamide |
| | 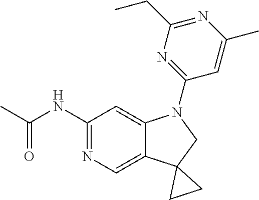 [SMILES: CCc1nc(C)cc(N2CC3(CC3)c3cnc(NC(C)=O)cc32)n1] |
| | SM: 6′-chloro-1′-(2-ethyl-6-methylpyrimidin-4-yl)-1′,2′- |
| | dihydrospiro[cyclopropane-1,3′-pyrrolo[3,2-c]pyridine] (Preparation 111). |
| | LCMS m/z = 324.2 [M + H]⁺; ¹H NMR (400 MHz, CDCl₃) δ 9.15 (br s, 1H), |
| | 8.26 (br s, 1H), 7.47 (s, 1H), 6.37 (s, 1H), 4.08 (s, 2H), 2.97 (q, 2H, J = 7.5 |
| | Hz), 2.47 (s, 3H), 2.21 (s, 3H), 1.47 (t, 3H, J = 7.6 Hz), 1.16 (br d, |
| | 4H, J = 11.3 Hz). |
| 65 | N-(1′-(6-methyl-2-propylpyrimidin-4-yl)-1′,2′-dihydrospiro[cyclopropane- |
| | 1,3′-pyrrolo[3,2-c]pyridin]-6′-yl)acetamide |
| | 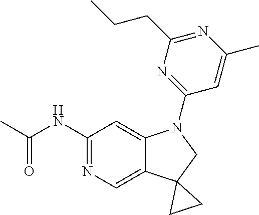 [SMILES: CCCc1nc(C)cc(N2CC3(CC3)c3cnc(NC(C)=O)cc32)n1] |
| | SM: 6′-chloro-1′-(6-methyl-2-propylpyrimidin-4-yl)-1′,2′- |
| | dihydrospiro[cyclopropane-1,3′-pyrrolo[3,2-c]pyridine] (Preparation 112) |
| | ¹H NMR (400 MHz, CDCl₃) δ: 9.10 (br s, 1H), 8.26 (br s, 1H), 7.46 (s, 1H), |
| | 6.40 (s, 1H), 4.09 (s, 2H), 3.00-2.90 (m, 2H), 2.47 (s, 3H), 2.21 (s, 3H), |
| | 2.00-1.90 (m, 2H), 1.20-1.10 (m, 4H), 1.03 (t, 3H, J = 7.4 Hz). |
| 66 | N-(1′-(2-isopropyl-6-methylpyrimidin-4-yl)-1′,2′- |
| | dihydrospiro[cyclopropane-1,3′-pyrrolo[3,2-c]pyridin]-6′-yl)acetamide |
| | 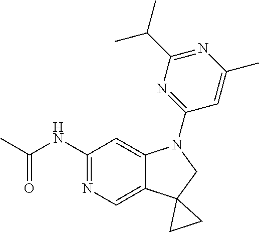 [SMILES: CC(=O)Nc1cc2c(cn1)C1(CC1)CN2c1cc(C)nc(C(C)C)n1] |
| | SM: 6′-chloro-1′-(2-isopropyl-6-methylpyrimidin-4-yl)-1′,2′- |
| | dihydrospiro[cyclopropane-1,3′-pyrrolo[3,2-c]pyridine] (Preparation 113). |
| | LCMS m/z = 338.1 [M + H]⁺; ¹H NMR (400 MHz, CDCl₃) δ 9.12 (br s, 1H), |
| | 8.20 (br s, 1H), 7.47 (s, 1H), 6.35 (s, 1H), 4.08 (s, 2H), 3.17 (quin, |
| | 1H, J = 6.9 Hz), 2.47 (s, 3H), 2.21 (s, 3H), 1.45 (d, 6H, J = 7.0 Hz), 1.20-1.10 |
| | (m, 4H). |
| 67 | N-(1′-(5-fluoro-2,6-dimethylpyrimidin-4-yl)-1′,2′- |
| | dihydrospiro[cyclopropane-1,3′-pyrrolo[3,2-c]pyridin]-6′-yl)acetamide |
| | 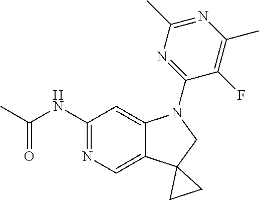 [SMILES: CC(=O)Nc1cc2c(cn1)C1(CC1)CN2c1nc(C)nc(C)c1F] |
| | SM: 6′-chloro-1′-(5-fluoro-2,6-dimethylpyrimidin-4-yl)-1′,2′- |
| | dihydrospiro[cyclopropane-1,3′-pyrrolo[3,2-c]pyridine] (Preparation 116). |
| | LCMS m/z = 328.1 [M + H]⁺ ¹H NMR (400 MHz, CDCl₃) δ 9.10-8.80 (m, |
| | 1H), 7.50-7.40 (m, 1H), 4.36 (d, 2H, J = 4.3 Hz), 2.69 (s, 3H), 2.47 (d, |
| | 3H, J = 3.3 Hz), 2.23 (s, 3H), 1.16 (s, 4H). |
| 68 | N-(1′-(2,6-dimethylpyrimidin-4-yl)-1′,2′-dihydrospiro[cyclopropane-1,3′- |
| | pyrrolo[3,2-c]pyridin]-6′-yl)acetamide |
| | 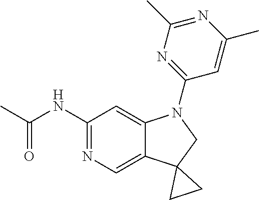 [SMILES: CC(=O)Nc1cc2c(cn1)C1(CC1)CN2c1cc(C)nc(C)n1] |
| | SM: 6′-chloro-1′-(2,6-dimethylpyrimidin-4-yl)-1′,2′- |
| | dihydrospiro[cyclopropane-1,3′-pyrrolo[3,2-c]pyridine] (Preparation 117). |
| | LCMS m/z = 328.1 [M + H]⁺ ¹H NMR (400 MHz, CDCl₃) δ 9.21 (br s, 1H), |
| | 8.30-8.00 (m, 1H), 7.46 (s, 1H), 6.50-6.30 (m, 1H), 4.09 (s, 2H), 2.73 (s, |
| | 3H), 2.47 (s, 3H), 2.23 (s, 3H), 1.20-1.10 (m, 4H). |
| 69 | N-(1′-(6-methyl-2-(1-methylcyclopropyl)pyrimidin-4-yl)-1′,2′- |
| | dihydrospiro[cyclopropane-1,3′-pyrrolo[3,2-c]pyridin]-6′-yl)acetamide |
| | 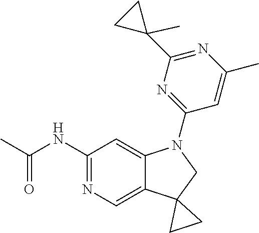 [SMILES: CC(=O)Nc1cc2c(cn1)C1(CC1)CN2c1cc(C)nc(C2(C)CC2)n1] |
| | SM: 6′-chloro-1′-(6-methyl-2-(1-methylcyclopropyl)pyrimidin-4-yl)-1′,2′- |
| | dihydrospiro[cyclopropane-1,3′-pyrrolo[3,2-c]pyridine] (Preparation 118). |
| | LCMS m/z =350.2 [M + H]⁺; ¹H NMR (400 MHz, CDCl₃) δ 9.00-8.80 (m, |
| | 1H), 8.18 (br s, 1H), 7.45 (s, 1H), 6.30 (s, 1H), 4.07 (s, 2H), 2.43 (s, 3H), |
| | 2.21 (s, 3H), 1.65 (s, 3H), 1.50-1.45 (m, 2H), 1.20-1.10 (m, 4H), 0.92-0.87 |
| | (m, 2H). |
| 70 | N-(1′-(6-methyl-2-(tetrahydro-2H-pyran-3-yl)pyrimidin-4-yl)-1′,2′- |
| | dihydrospiro[cyclopropane-1,3′-pyrrolo[3,2-c]pyridin]-6′-yl)acetamide |
| | 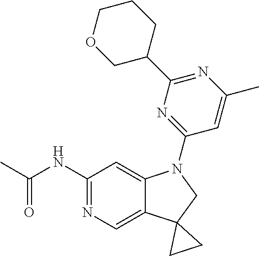 [SMILES: CC(=O)Nc1cc2c(cn1)C1(CC1)CN2c1cc(C)nc(C2CCCOC2)n1] |
| | SM: 6′-chloro-1′-(6-methyl-2-(tetrahydro-2H-pyran-3-yl)pyrimidin-4-yl)- |
| | 1′,2′-dihydrospiro[cyclopropane-1,3′-pyrrolo[3,2-c]pyridine] (Preparation |
| | 119). |
| | LCMS m/z = 380.2 [M + H]⁺; ¹H NMR (400 MHz, CDCl₃) δ 9.09 (br s, 1H), |
| | 8.27 (br s, 1H), 7.48 (s, 1H), 6.40-6.30 (m, 1H), 4.22 (ddd, 1H, J = 1.5, 4.1, |
| | 10.9 Hz), 4.10-4.00 (m, 2H), 4.00-3.90 (m, 2H), 3.72 (dt, 1H, J = 3.0, 11.1 |
| | Hz), 3.15 (tt, 1H, J = 4.2, 10.9 Hz), 2.46 (s, 3H), 2.30-2.10 (m, 5H), 1.90-1.70 |
| | (m, 2H), 1.20-1.10 (m, 4H). |
| 71 | N-(1′-(2-(3-methoxypropyl)-6-methylpyrimidin-4-yl)-1′,2′- |
| | dihydrospiro[cyclopropane-1,3′-pyrrolo[3,2-c]pyridin]-6′-yl)acetamide |
| | 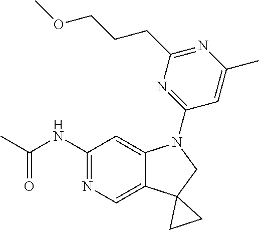 [SMILES: COCCCc1nc(C)cc(N2CC3(CC3)c3cnc(NC(C)=O)cc32)n1] |
| | SM: 6′-chloro-1′-(2-(3-methoxypropyl)-6-methylpyrimidin-4-yl)-1′,2′- |
| | dihydrospiro[cyclopropane-1,3′-pyrrolo[3,2-c]pyridine] (Preparation 120). |
| | LCMS m/z = 368.2 [M + H]⁺ ¹H NMR (400 MHz, CDCl₃) δ 9.04 (br s, 1H), |
| | 8.37 (br s, 1H), 7.47 (s, 1H), 6.43 (s, 1H), 4.09 (s, 2H), 3.53 (t, 2H, J = 6.8 |
| | Hz), 3.35 (s, 3H), 2.98 (t, 2H, J = 7.6 Hz), 2.46 (s, 3H), 2.30-2.20 (m, 5H), |
| | 1.20-1.10 (m, 4H). |

### Example 72 and 73

### trans-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide and cis-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide

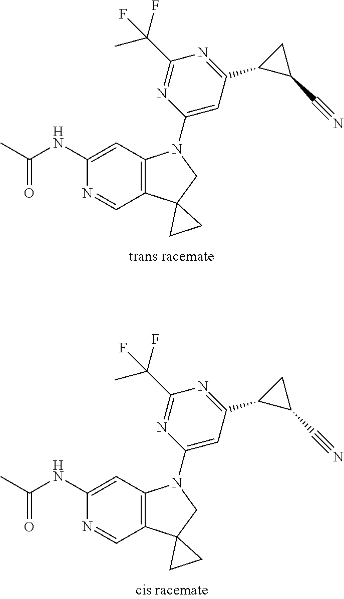 [SMILES: CC(=O)Nc1cc2c(cn1)C1(CC1)CN2c1cc([C@@H]2C[C@H]2C#N)nc(C(C)(F)F)n1 ; CC(=O)Nc1cc2c(cn1)C1(CC1)CN2c1cc([C@@H]2C[C@@H]2C#N)nc(C(C)(F)F)n1]

trans-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide and cis-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide were prepared from 2-(6-(6′-chlorospiro[cyclopropane-1,3′-pyrrolo[3,2-c]pyridin]-1′(2′H)-yl)-2-(1,1-difluoroethyl)pyrimidin-4-yl)cyclopropane-1-carbonitrile (Preparation 146) and acetamide, following a similar procedure to that described in Example 12.

Isomer 1, trans-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide or cis-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide, 13 mg, LCMS m/z=411.2 [M+H]⁺. ¹H NMR: (400 MHz, CDCl₃) δ: ppm 9.20-8.90 (m, 1H), 8.29 (br s, 1H), 7.52 (s, 1H), 6.73 (s, 1H), 4.20-4.10 (m, 2H), 2.61 (ddd, 1H, J=4.3, 5.8, 8.7 Hz), 2.30-2.20 (m, 4H), 2.13 (t, 3H, J=18.7 Hz), 1.85 (ddd, 1H, J=4.8, 5.8, 9.0 Hz), 1.67 (ddd, 1H, J=4.8, 6.0, 8.8 Hz), 1.22 (br d, 4H, J=4.3 Hz).

Isomer 2, cis-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide or trans-rac-N-(1′-(6-(2-cyanocyclopropyl)-2-(1,1-difluoroethyl)pyrimidin-4-yl)-1′,2′-dihydrospiro[cyclopropane-1,3′-pyrrolo[3,2-c]pyridin]-6′-yl)acetamide, 2 mg, LCMS m/z=411.2 [M+H]⁺. ¹H NMR: (400 MHz, CDCl₃) δ: ppm 9.01 (br s, 1H), 8.60-8.10 (m, 1H), 7.50-7.49 (m, 1H), 6.90-6.70 (m, 1H), 4.21-4.20 (m, 2H), 2.70-2.60 (m, 1H), 2.24 (s, 3H), 2.20-2.10 (m, 4H), 2.02-2.00 (m, 1H), 1.63 (br dd, 1H, J=3.4, 8.4 Hz), 1.21 (br s, 4H).

### Examples 74 and 75

### N-(1′-(6-((1s,3s)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide and N-(1′-(6-((1r,3r)-3-cyanocyclobutyl)-2-(1,1-difluoroethyl)pyrimidin-4-yl)-1′,2′-dihydrospiro[cyclopropane- 1,3′-pyrrolo[3,2-c]pyridin]-6′-yl)acetamide

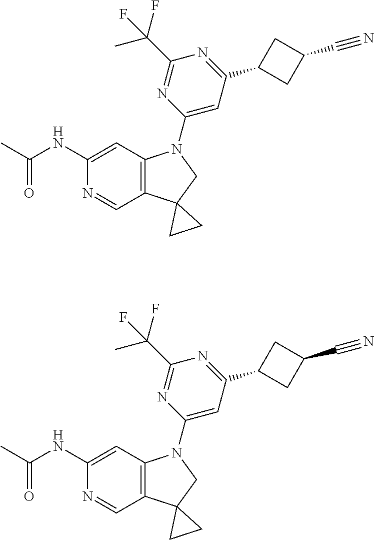 [SMILES: CC(=O)Nc1cc2c(cn1)C1(CC1)CN2c1cc([C@H]2C[C@@H](C#N)C2)nc(C(C)(F)F)n1 ; CC(=O)Nc1cc2c(cn1)C1(CC1)CN2c1cc([C@H]2C[C@H](C#N)C2)nc(C(C)(F)F)n1]

N-(1′-(6-((1s,3s)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide and N-(1′-(6-((1r,3r)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide were prepared from 3-(6-(6′-chlorospiro[cyclopropane-1,3′-pyrrolo[3,2-c]pyridin]-1′(2′H)-yl)-2-(1,1-difluoroethyl)pyrimidin-4-yl)cyclobutane-1-carbonitrile (Preparation 143) and acetamide following a similar procedure to that described in Example 12. Isomer 1, N-(1′-(6-((1s,3s)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide or N-(1′-(6-((1r,3r)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide, 11 mg, LCMS m/z=425.3 [M+H]⁺. ¹H NMR: (400 MHz, CDCl₃) δ: ppm 9.20-8.90 (m, 1H), 8.07 (br s, 1H), 7.52 (s, 1H), 6.55 (br s, 1H), 4.13 (s, 2H), 3.90-3.80 (m, 1H), 3.50-3.40 (m, 1H), 2.90-2.70 (m, 4H), 2.30-2.10 (m, 7H), 1.21-1.18 (m, 4H). Isomer 2, N-(1′-(6-((1r,3r)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide or N-(1′-(6-((1s,3s)-3-cyanocyclobutyl)-2-(1,1-difluoroethyl)pyrimidin-4-yl)-1′,2′-dihydrospiro[cyclopropane-1,3′-pyrrolo[3,2-c]pyridin]-6′-yl)acetamide, 16 mg, LCMS m/z=425.3 [M+H]⁺. ¹H NMR: (400 MHz, CDCl₃) δ: ppm 9.10-8.90 (m, 1H), 8.50-8.20 (m, 1H), 7.50 (s, 1H), 6.64 (s, 1H), 4.18 (s, 2H), 3.66 (quin, 1H, J=9.0 Hz), 3.20-3.10 (m, 1H), 2.83-2.80 (m, 4H), 2.20-2.10 (m, 7H), 1.21 (s, 4H).

### Example 76

### N-(1-(6-(1,1-difluoroethyl)pyridin-2-yl)-3,3-dimethyl-2,3-dihydro-1H-pyrrolo[3,2-c]pyridin-6-yl)acetamide trifluoroacetate

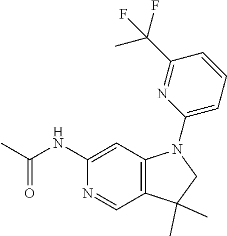 [SMILES: CC(=O)Nc1cc2c(cn1)C(C)(C)CN2c1cccc(C(C)(F)F)n1]

A mixture of 6-chloro-1-(6-(1,1-difluoroethyl)pyridin-2-yl)-3,3-dimethyl-2,3-dihydro-1H-pyrrolo[3,2-c]pyridine (Preparation 84, 30 mg, 92.7 μmol), acetamide (27.37 mg, 463.3 μmol), Cs₂CO₃ (60.38 mg, 185.3 μmol) and BrettPhos Pd G3 (4.20 mg, 4.63 μmol) in dioxane (1 mL, 11.74 mmol) was purged with N₂, then heated at 110° C. in a sealed tube for 1 h. The cooled mixture was filtered and the resulting crude solid was purified by HPLC, eluting with MeCN, water (+0.1% TFA modifier) to give N-(1-(6-(1,1-difluoroethyl)pyridin-2-yl)-3,3-dimethyl-2,3-dihydro-1H-pyrrolo[3,2-c]pyridin-6-yl)acetamide trifluoroacetate (5 mg, 12% yield) as a white powder. LCMS m/z=347.2 [M+H]⁺; ¹H NMR (400 MHz, CDCl₃) δ 12.35 (br d, 1H, J=2.5 Hz), 9.20-9.00 (m, 1H), 8.00-7.90 (m, 1H), 7.68 (s, 1H), 7.60-7.40 (m, 1H), 7.06 (br d, 1H, J=7.3 Hz), 4.06 (s, 2H), 2.34 (s, 3H), 2.18 (br t, 3H, J=18.9 Hz), 1.49 (s, 6H).

### Example 77

### N-(1′-(thiazol-2-yl)-1′,2′-dihydrospiro[cyclopropane-1,3′-pyrrolo[3,2-c]pyridin]-6′-yl)acetamide trifluoroacetate

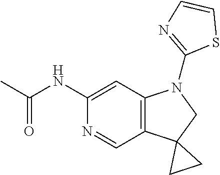 [SMILES: CC(=O)Nc1cc2c(cn1)C1(CC1)CN2c1nccs1]

To a mixture of 2-(6′-chlorospiro[cyclopropane-1,3′-pyrrolo[3,2-c]pyridin]-1′(2′H)-yl)thiazole (Preparation 100, 69.2 mg, 262.37 μmol), acetamide (77.49 mg, 1.31 mmol) and Cs₂CO₃ (170.97 mg, 524.75 μmol) in dioxane (3.22 mL) was added BrettPhos Pd G3 (23.78 mg, 26.24 μmol) and the mixture was purged with N₂, then stirred at 100° C. overnight. The cooled reaction was diluted with EtOAc, washed with NaHCO₃, H₂O, and brine. The organic layer was dried over Na₂SO₄ and the solvent removed in vacuo. The crude mixture was purified by reversed phase HPLC (Sunfire Prep C18 OBD 5 um 30×50 mm, Gradient: 5-40% MeCN in water, modifier 0.1% TFA, Flow rate: 50 ml/min to yield N-(1′-(thiazol-2-yl)-1′,2′ -dihydrospiro[cyclopropane-1,3′-pyrrolo[3,2-c]pyridin]-6′-yl)acetamide trifluoroacetate, 2.5 mg. LCMS m/z=287 [M+H]⁺

### Examples 78 to 82

The compounds in the following table were prepared from the appropriate chloro compound and acetamide, following the procedure described in Example 77.

| Example No | Name, Structure, Starting material (SM), HPLC, Data |
|---|---|
| 78 | N-(1′-(5-methylthiazol-2-yl)-1′,2′-dihydrospiro[cyclopropane-1,3′- |
| | pyrrolo[3,2-c]pyridin]-6′-yl)acetamide trifluoroacetate |
| | 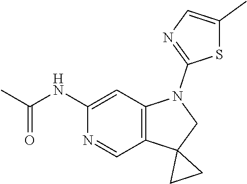 [SMILES: CC(=O)Nc1cc2c(cn1)C1(CC1)CN2c1ncc(C)s1] |
| | SM: 2-(6′-chlorospiro[cyclopropane-1,3′-pyrrolo[3,2-c]pyridin]-1′(2′H)- |
| | yl)-5-methylthiazole (Preparation 122) |
| | 13.7 mg, LCMS m/z = 301 [M + H]⁺ |
| 79 | N-(1′-(4-(1,1-difluoroethyl)thiazol-2-yl)-1′,2′-dihydrospiro[cyclopropane- |
| | 1,3′-pyrrolo[3,2-c]pyridin]-6′-yl)acetamide trifluoroacetate |
| | 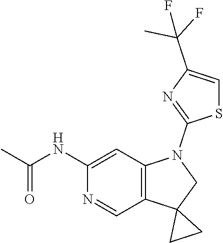 [SMILES: CC(=O)Nc1cc2c(cn1)C1(CC1)CN2c1nc(C(C)(F)F)cs1] |
| | SM: 2-(6′-chlorospiro[cyclopropane-1,3′-pyrrolo[3,2-c]pyridin]-1′(2′H)- |
| | yl)-4-(1,1-difluoroethyl)thiazole (Preparation 123) |
| | 7.5 mg, LCMS m/z = 351 [M + H]⁺ |
| 80^{A} | N-(1′-(4,5-dimethylthiazol-2-yl)-1′,2′-dihydrospiro[cyclopropane-1,3′- |
| | pyrrolo[3,2-c]pyridin]-6′-yl)acetamide |
| | 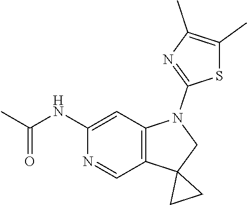 [SMILES: CC(=O)Nc1cc2c(cn1)C1(CC1)CN2c1nc(C)c(C)s1] |
| | SM: 2-(6′-chlorospiro[cyclopropane-1,3′-pyrrolo[3,2-c]pyridin]-1′(2′H)- |
| | yl)-4,5-dimethylthiazole (Preparation 124) |
| | 3.9 mg, LCMS m/z = 315 [M + H]⁺. |
| 81 | N-(1′-(4-(tetrahydro-2H-pyran-3-yl)thiazol-2-yl)-1′,2′- |
| | dihydrospiro[cyclopropane-1,3′-pyrrolo[3,2-c]pyridin]-6′-yl)acetamide |
| | trifluoroacetate |
| | 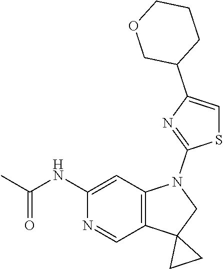 [SMILES: CC(=O)Nc1cc2c(cn1)C1(CC1)CN2c1nc(C2CCCOC2)cs1] |
| | SM: 2-(6′-chlorospiro[cyclopropane-1,3′-pyrrolo[3,2-c]pyridin]-1′(2′H)- |
| | yl)-4-(tetrahydro-2H-pyran-3-yl)thiazole (Preparation 125) |
| | 28.9 mg, LCMS m/z = 371 [M + H]⁺ |
| 82 | N-(1′-(5-methyl-4-(tetrahydrofuran-3-yl)thiazol-2-yl)-1′,2′- |
| | dihydrospiro[cyclopropane-1,3′-pyrrolo[3,2-c]pyridin]-6′-yl)acetamide |
| | trifluoroacetate |
| | 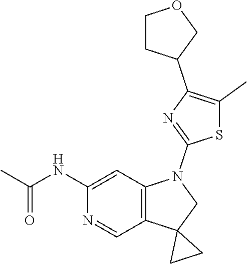 [SMILES: CC(=O)Nc1cc2c(cn1)C1(CC1)CN2c1nc(C2CCOC2)c(C)s1] |
| | 2-(6′-chlorospiro[cyclopropane-1,3′-pyrrolo[3,2-c]pyridin]-1′(2′H)-yl)-5- |
| | methyl-4-(tetrahydrofuran-3-yl)thiazole (Preparation 126) |
| | 13.6 mg, LCMS m/z = 371 [M + H]⁺ |

A crude purified by HPLC (Column: XSelect CSH Prep C18 5 um OBD 30×50 mm) Gradient: 5-70 MeCN in water, modifier 0.2% NH₄OH, Flow rate: 50 ml/min

### Example 83

### N-(1′-(4-(1,1-difluoroethyl)-6-methylpyrimidin-2-yl)-1′,2′-dihydrospiro[cyclopropane-1,3′-pyrrolo[3,2-c]pyridin]-6′-yl)acetamide

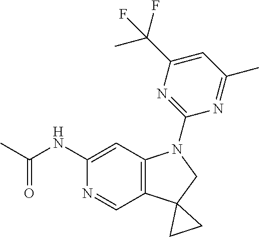 [SMILES: CC(=O)Nc1cc2c(cn1)C1(CC1)CN2c1nc(C)cc(C(C)(F)F)n1]

To a solution of 6′-chloro-1′-(4-(1,1-difluoroethyl)-6-methylpyrimidin-2-yl)-1′,2′-dihydrospiro[cyclopropane-1,3′-pyrrolo[3,2-c]pyridine] (Preparation 75, 130 mg, 0.386 mmol) in dioxane (3 mL) was added acetamide (34.2 mg, 0.579 mmol), Cs₂CO₃ (252 mg, 0.772 mmol), Pd₂(dba)₃ (70.7 mg, 0.077 mmol) and Xantphos (89.3 mg, 0.154 mmol). The reaction mixture was stirred at 100° C. for 3 h under N₂. The mixture was concentrated and purified by Prep-HPLC (Welch Xtimate C18 150*25 mm*5 um; Condition: water (10 mM NH₄HCO₃)-MeCN; Begin B: 40; End B: 70; Gradient Time (min): 11; Flow Rate (ml/min): 25) to give N-(1′-(4-(1,1-difluoroethyl)-6-methylpyrimidin-2-yl)-1′,2′-dihydrospiro[cyclopropane-1,3′-pyrrolo[3,2-c]pyridin]-6′-yl)acetamide (37.1 mg, 26.7% yield) as a yellow solid. LCMS m/z=360.2 [M+H]⁺. ¹H NMR: (400 MHz, CDCl₃) δ: ppm 9.13 (s, 1H), 7.89 (s, 1H), 7.47 (s, 1H), 7.03 (s, 1H), 4.29 (s, 2H), 2.57 (s, 3H), 2.20 (s, 3H), 2.12 (t, J=18.8 Hz, 3H), 1.14 (s, 4H).

### Examples 84 to 87

The compounds in the following table were prepared from the appropriate chloro compound and acetamide, following a similar procedure to that described in Example 83.

| Example No | Name, Structure, Starting material (SM), Data |
|---|---|
| 84 | N-(1′-(6-(oxetan-3-yl)pyridin-2-yl)-1′,2′-dihydrospiro[cyclopropane-1,3′- |
| | pyrrolo[3,2-c]pyridin]-6′-yl)acetamide |
| | 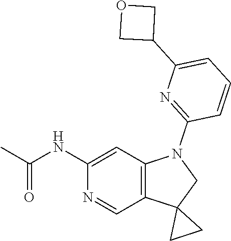 [SMILES: CC(=O)Nc1cc2c(cn1)C1(CC1)CN2c1cccc(C2COC2)n1] |
| | SM: 6′-chloro-1′-(6-(oxetan-3-yl)pyridin-2-yl)-1′,2′- |
| | dihydrospiro[cyclopropane-1,3′-pyrrolo[3,2-c]pyridine] (Preparation 59) |
| | 31 mg, 28.9% yield, white solid. |
| | LCMS m/z = 337.4 [M + H]⁺. ¹H NMR: (500 MHz, CDCl₃) δ: ppm 8.83 (s, |
| | 1H), 8.37 (s, 1H), 7.68-7.61 (m, 1H), 7.42 (s, 1H), 6.87 (d, J = 7.5 Hz, 1H), |
| | 6.76 (d, J = 8.0 Hz, 1H), 5.11-5.03 (m, 4H), 4.46-4.39 (m, 1H), 4.16 (s, 2H), |
| | 2.18 (s, 3H), 1.17-1.10 (m, 4H). |
| 85 | N-(1′-(6-(3-fluorooxetan-3-yl)pyridin-2-yl)-1′,2′-dihydrospiro[cyclopropane- |
| | 1,3′-pyrrolo[3,2-c]pyridin]-6′-yl)acetamide |
| | 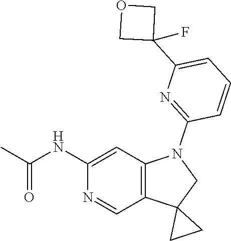 [SMILES: CC(=O)Nc1cc2c(cn1)C1(CC1)CN2c1cccc(C2(F)COC2)n1] |
| | SM: 6′-chloro-1′-(6-(3-fluorooxetan-3-yl)pyridin-2-yl)-1′,2′- |
| | dihydrospiro[cyclopropane-1,3′-pyrrolo[3,2-c]pyridine] (Preparation 60) |
| | 19.3 mg, 12.0% yield, white solid. |
| | LCMS m/z = 355.1 [M + H]⁺. ¹H NMR: (400 MHz, CDCl₃) δ: ppm 8.72 (s, |
| | 1H), 8.09 (s, 1H), 7.74-7.70 (m, 1H), 7.46 (s, 1H), 7.11 (d, J = 7.2 Hz, 1H), |
| | 6.92 (d, J = 8.0 Hz, 1H), 5.35-5.27 (m, 2H), 5.06-4.99 (m, 2H), 4.19 (s, 2H), |
| | 2.18 (s, 3H), 1.19-1.15 (m, 4H). |
| 86 | N-(1′-(6-(1,1-difluoroethyl)-4-(2-methoxyethoxy)pyridin-2-yl)-1′,2′- |
| | dihydrospiro[cyclopropane-1,3′-pyrrolo[3,2-c]pyridin]-6′-yl)acetamide |
| | 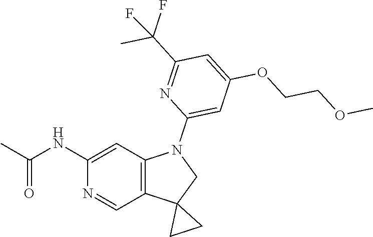 [SMILES: COCCOc1cc(N2CC3(CC3)c3cnc(NC(C)=O)cc32)nc(C(C)(F)F)c1] |
| | SM: 6′-chloro-1′-(6-(1,1-difluoroethyl)-4-(2-methoxyethoxy)pyridin-2-yl)- |
| | 1′,2′-dihydrospiro[cyclopropane-1,3′-pyrrolo[3,2-c]pyridine] |
| | (Preparation 63) |
| | 11.5 mg, 16.7% yield, white solid. |
| | LCMS m/z = 419.1 [M + H]⁺. ¹H NMR: (400 MHz, CDCl₃) δ: ppm 8.86 (s, |
| | 1H), 8.07 (s, 1H), 7.41 (s, 1H), 6.84 (s, 1H), 6.35 (s, 1H), 4.25-4.23 (m, 2H), |
| | 4.07 (s, 2H), 3.79-3.76 (m, 2H), 3.46 (s, 3H), 2.22-2.12 (m, 6H), 1.15-1.11 |
| | (m, 4H). |
| 87 | N-(1′-(6-(3-methoxyoxetan-3-yl)pyridin-2-yl)-1′,2′- |
| | dihydrospiro[cyclopropane-1,3′-pyrrolo[3,2-c]pyridin]-6′-yl)acetamide |
| | 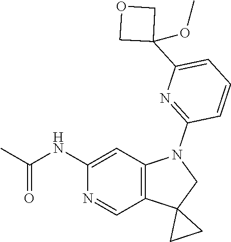 [SMILES: COC1(c2cccc(N3CC4(CC4)c4cnc(NC(C)=O)cc43)n2)COC1] |
| | SM: 6′-chloro-1′-(6-(3-methoxyoxetan-3-yl)pyridin-2-yl)-1′,2′- |
| | dihydrospiro[cyclopropane-1,3′-pyrrolo[3,2-c]pyridine] (Preparation 76) |
| | 35.5 mg, 33.3% yield, white solid. |
| | LCMS m/z = 367.1 [M + H]⁺. ¹H NMR: (500 MHz, CDCl₃) δ: ppm 8.13 (s, |
| | 1H), 7.90 (s, 1H), 7.73 (t, J = 8.0 Hz, 1H), 7.44 (s, 1H), 7.02 (d, J = 7.5 Hz, |
| | 1H), 6.86 (d, J = 7.5 Hz, 1H), 5.18 (d, J-6.5 Hz, 2H), 4.93 (d, J = 6.5 Hz, 2H), |
| | 4.18 (s, 2H), 3.26 (s, 3H), 2.18 (s, 3H), 1.18-1.12 (m, 4H). |

### Example 88

### N-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)acetamide

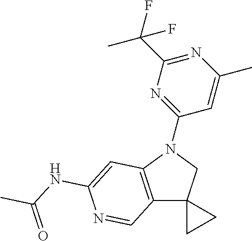 [SMILES: CC(=O)Nc1cc2c(cn1)C1(CC1)CN2c1cc(C)nc(C(C)(F)F)n1]

To a solution of 6′-chloro-1′-(2-(1,1-difluoroethyl)-6-methylpyrimidin-4-yl)-1′,2′-dihydrospiro[cyclopropane-1,3′-pyrrolo[3,2-c]pyridine] (Preparation 73, 2.15 g, 6.38 mmol), acetamide (1.9 g, 31.9 mmol) and Cs₂CO₃ (4.2 g, 12.8 mmol) in dioxane (20 mL) was added Pd₂(dba)₃ (1.2 g, 1.28 mmol) and Xantphos (1.5 g, 2.55 mmol). The reaction mixture was stirred at 120° C. for 12 h under N₂. The mixture was poured into H₂O (50 mL) and extracted with EtOAc (50 mL×3). The organic phase was washed with brine (50 mL×2), dried over Na₂SO₄ and filtered. The filtrate was concentrated and the residue was purified by chromatography on silica gel (PE/EtOAc=20/1 to 0/1) to give a crude which was treated with PE (20 mL) and MeOH (5 mL) to give N-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)acetamide (1.21 g, 52.7% yield) as a white solid. LCMS m/z=360.2 [M+H]⁺. ¹H NMR: (400 MHz, DMSO-d₆) δ: ppm 10.30 (s, 1H), 9.01 (s, 1H), 7.69 (s, 1H), 6.80 (s, 1H), 4.14 (s, 2H), 2.46 (s, 3H), 2.15 (t, J=19.2 Hz, 3H), 2.07 (s, 3H), 1.18-1.16 (m, 4H).

### Example 89

### N-(1′-(2-(1,1-difluoroethyl)pyrimidin-4-yl)-1′,2′-dihydrospiro[cyclopropane-1,3′-pyrrolo[3,2-c]pyridin]-6′-yl)propionamide

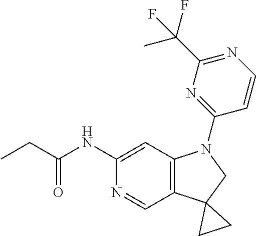 [SMILES: CCC(=O)Nc1cc2c(cn1)C1(CC1)CN2c1ccnc(C(C)(F)F)n1]

A solution of 6′-chloro-1′-(2-(1,1-difluoroethyl)pyrimidin-4-yl)-1′,2′-dihydrospiro[cyclopropane-1,3′-pyrrolo[3,2-c]pyridine] (Preparation 85, 120 mg, 0.372 mmol), propionamide (27.2 mg, 0.372 mmol), Cs₂CO₃ (363 mg, 1.12 mmol) in dioxane (10 mL) was added BrettPhos Pd G3 (33.8 mg, 0.0372 mmol). The reaction mixture was stirred at 100° C. for 2 h under N₂. The mixture was treated with H₂O (50 mL) and extracted with EtOAc (20 mL×3). The combined organic layers were washed with brine (20 mL), dried over Na₂SO₄, filtered and concentrated under vacuum to give the crude product which was purified by prep-HPLC (Column: Boston Prime C18 150*30 mm*5 um; Condition: water(NH₃·H₂O+NH₄HCO₃)-ACN; Begin B: 41; End B: 71; Gradient Time (min): 10; Flow Rate (ml/min): 25) to give N-(1′-(2-(1,1-difluoroethyl)pyrimidin-4-yl)-1′,2′-dihydrospiro[cyclopropane-1,3′-pyrrolo[3,2-c]pyridin]-6′-yl)propionamide (56 mg, 41.9% yield) as a white solid. LCMS m/z=360.1 [M+H]⁺. ¹H NMR: (500 MHz, CDCl₃) δ: ppm 8.97 (s, 1H), 8.61 (d, J=6.0 Hz, 1H), 7.86 (s, 1H), 7.53 (s, 1H), 6.82 (d, J=5.5 Hz, 1H), 4.16 (s, 2H), 2.46-2.41 (m, 2H), 2.19 (t, J=19.0 Hz, 3H), 1.27 (t, J=7.5 Hz, 3H), 1.22-1.21 (m, 2H), 1.20-1.18 (m, 2H).

### Example 90

### N-(1′-(6-(1,1-difluoroethyl)pyridin-2-yl)-1′,2′-dihydrospiro[cyclopropane-1,3′-pyrrolo[3,2-c]pyridin]-6′-yl)propionamide

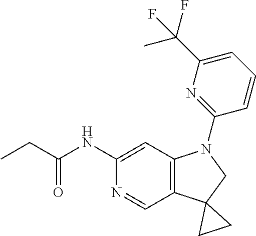 [SMILES: CCC(=O)Nc1cc2c(cn1)C1(CC1)CN2c1cccc(C(C)(F)F)n1]

N-(1′-(6-(1,1-difluoroethyl)pyridin-2-yl)-1′,2′-dihydrospiro[cyclopropane-1,3′-pyrrolo[3,2-c]pyridin]-6′-yl)propionamide was obtained, 20 mg, from 6′-chloro-1′-(6-(1,1-difluoroethyl)pyridin-2-yl)-1′,2′-dihydrospiro[cyclopropane-1,3′-pyrrolo[3,2-c]pyridine] (Preparation 77) and propionamide, following the procedure described in Example 12. LCMS m/z=359.2 [M+H]⁺. ¹H NMR: (400 MHz, CDCl₃) δ: ppm 8.81 (s, 1H), 7.99 (br s, 1H), 7.78 (t, 1H, J=7.9 Hz), 7.44 (s, 1H), 7.30-7.28 (m, 1H), 7.00-6.90 (m, 1H), 4.16 (s, 2H), 2.42 (q, 2H, J=7.5 Hz), 2.19 (t, 3H, J=18.9 Hz), 1.26 (t, 3H, J=7.5 Hz), 1.20-1.10 (m, 4H).

### Example 91

### N-(1′-(2-(1,1-difluoroethyl)pyrimidin-4-yl)-1′,2′-dihydrospiro[cyclopropane-1,3′-pyrrolo[3,2-c]pyridin]-6′-yl)-3-methoxypropanamide

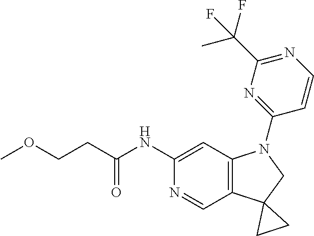 [SMILES: COCCC(=O)Nc1cc2c(cn1)C1(CC1)CN2c1ccnc(C(C)(F)F)n1]

To a solution of 6′-chloro-1′-(2-(1,1-difluoroethyl)pyrimidin-4-yl)-1′,2′-dihydrospiro[cyclopropane-1,3′-pyrrolo[3,2-c]pyridine] (Preparation 85, 110 mg, 0.341 mmol), 3-methoxypropanamide (70.3 mg, 0.682 mmol) and Cs₂CO₃ (333 mg, 1.02 mmol) in dioxane (10 mL) was added BrettPhos Pd G3 (30.9 mg, 0.0341 mmol). The reaction mixture was stirred at 100° C. under N₂. The mixture was treated with H₂O (50 mL) and extracted with EtOAc (20 mL×3). The combined organic layers were washed with brine (20 mL), dried over Na₂SO₄, filtered and concentrated under vacuum to give the crude product which was purified by prep-HPLC (Column: Welch Xtimate C18 150*25 mm*5 um; Condition: water(NH₄HCO₃)-ACN; Begin B: 26; End B: 60; Gradient Time (min): 11; Flow Rate (ml/min): 25) to give N-(1′-(2-(1,1-difluoroethyl)pyrimidin-4-yl)-1′,2′-dihydrospiro[cyclopropane-1,3′-pyrrolo[3,2-c]pyridin]-6′-yl)-3-methoxypropanamide (32.0 mg, 24.1% yield) as a white solid. LCMS m/z=390.1 [M+H]⁺. ¹H NMR: (500 MHz, CDCl₃) δ: ppm 9.02 (s, 1H), 8.88 (s, 1H), 8.61 (d, J=6.0 Hz, 1H), 7.54 (s, 1H), 6.78 (d, J=5.5 Hz, 1H), 4.14 (s, 2H), 3.76-3.73 (m, 2H), 3.45 (s, 3H), 2.69-2.66 (m, 2H), 2.19 (d, J=18.5 Hz, 3H), 1.20-1.18 (m, 4H).

### Example 92

### N-(1′-(6-(1,1-difluoroethyl)-4-((1r,3r)-3-methoxycyclobutoxy)pyridin-2-yl)-1′,2′-dihydrospiro[cyclopropane-1,3′-pyrrolo[3,2-c]pyridin]-6′-yl)acetamide

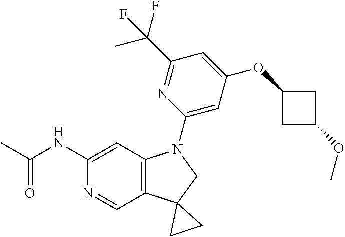 [SMILES: CO[C@H]1C[C@H](Oc2cc(N3CC4(CC4)c4cnc(NC(C)=O)cc43)nc(C(C)(F)F)c2)C1]

To a solution of N-(1′,2′-dihydrospiro[cyclopropane-1,3′-pyrrolo[3,2-c]pyridin]-6′-yl)acetamide (Preparation 7, 60.0 mg, 0.295 mmol) in dioxane (3 mL) was added 2-chloro-6-(1,1-difluoroethyl)-4-((1r,3r)-3-methoxycyclobutoxy)pyridine (Preparation 34, 98.4 mg, 0.354 mmol), Xantphos Pd G₃ (28.4 mg, 0.0295 mmol) and Zn(OAc)₂ (162 mg, 0.886 mmol). The reaction mixture was stirred at 100° C. for 1 h under N₂. The mixture was concentrated and purified by prep-HPLC (Column: Boston Green ODS 150*30 mm*5 um; Condition: water (10 mM NH₄HCO₃)-ACN; Begin B: 49; End B: 79; Gradient Time (min): 11; Flow Rate (ml/min): 25; to give N-(1′-(6-(1,1-difluoroethyl)-4-((1r,3r)-3-methoxycyclobutoxy)pyridin-2-yl)-1′,2′-dihydrospiro[cyclopropane-1,3′-pyrrolo[3,2-c]pyridin]-6′-yl)acetamide (13.6 mg, 10.4% yield) as a white solid. LCMS m/z=445.2 [M+H]⁺. ¹H NMR: (500 MHz, CDCl₃) δ: ppm 8.81 (s, 1H), 8.06 (s, 1H), 7.41 (s, 1H), 6.76 (s, 1H), 6.28 (s, 1H), 4.49-4.43 (m, 1H), 4.08 (s, 2H), 3.72-3.70 (m, 1H), 3.27 (s, 3H), 2.99-2.93 (m, 2H), 2.20-2.11 (m, 8H), 1.17-1.06 (m, 4H).

### Example 93

### N-(1′-(6-(methylsulfonyl)pyridin-2-yl)-1′,2′-dihydrospiro[cyclopropane-1,3′-pyrrolo[3,2-c]pyridin]-6′-yl)acetamide

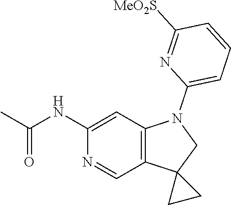 [SMILES: CC(=O)Nc1cc2c(cn1)C1(CC1)CN2c1cccc(S(C)(=O)=O)n1]

To a mixture of N-(1′,2′-dihydrospiro[cyclopropane-1,3′-pyrrolo[3,2-c]pyridin]-6′-yl)acetamide

(Preparation 7, 60 mg, 0.295 mmol), 2-bromo-6-methylsulfonyl-pyridine (139 mg, 0.59 mmol), BrettPhos Pd G3 (26.8 mg, 0.030 mmol) and Cs₂CO₃ (192.38 mg, 0.59 mmol) was added dioxane (2.5 mL) and the reaction was heated at 90° C. for 1 h and then stirred at rt overnight. The mixture was diluted with EtOAc and water, the layers separated and the aqueous layer was extracted with EtOAc. The combined organic layers were concentrated and dry-loaded and purified by chromatography on silica gel (20-100% EtOAc-EtOH 3:1 in heptane) to give N-(1′-(6-(methylsulfonyl)pyridin-2-yl)-1′,2′-dihydrospiro[cyclopropane-1,3′-pyrrolo[3,2-c]pyridin]-6′-yl)acetamide (15 mg, 14.2% yield) as a white solid. LCMS m/z=359.1 [M+H]⁺. ¹H NMR (400 MHz, CDCl₃): δ (ppm) 9.16 (s, 1H), 8.13-8.07 (m, 1H), 7.94 (d, J=7.5 Hz, 1H), 7.25-7.19 (m, 2H), 4.36 (s, 2H), 3.47 (s, 3H), 2.37 (s, 3H), 1.40-1.35 (m, 2H), 1.31-1.26 (m, 2H)

### Example 94

### N-(1′-(pyridin-2-yl)-1′,2′-dihydrospiro[cyclopropane-1,3′-pyrrolo[3,2-c]pyridin]-6′-yl)acetamide

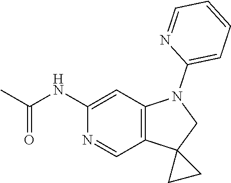 [SMILES: CC(=O)Nc1cc2c(cn1)C1(CC1)CN2c1ccccn1]

N-(1′-(pyridin-2-yl)-1′,2′-dihydrospiro[cyclopropane-1,3′-pyrrolo[3,2-c]pyridin]-6′-yl)acetamide

was obtained as a white solid, 6 mg, 7.8% yield, from N-(1′,2′-dihydrospiro[cyclopropane-1,3′-pyrrolo[3,2-c]pyridin]-6′-yl)acetamide (Preparation 7) and 2-bromopyridine following a similar procedure to that described in Example 93. LCMS m/z=281.0 [M+H]⁺ 1H NMR (400 MHz, MeOH-d₄): δ (ppm) 8.68 (s, 1H), 8.37 (ddd, J=5.0, 2.0, 1.0 Hz, 1H), 7.75 (ddd, J= 8.3, 7.3, 2.0 Hz, 1H), 7.47 (s, 1H), 7.01-6.93 (m, 2H), 4.12 (s, 2H), 2.17 (s, 3H), 1.17 (d, J=3.5 Hz, 4H)

### Example 95

### N-(1′-(6-(3-hydroxytetrahydrofuran-3-yl)-4-methylpyridin-2-yl)-1′,2′-dihydrospiro[cyclopropane-1,3′-pyrrolo[3,2-c]pyridin]-6′-yl)acetamide

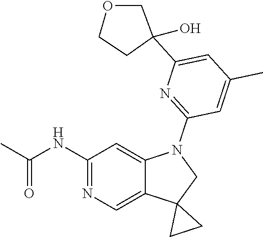 [SMILES: CC(=O)Nc1cc2c(cn1)C1(CC1)CN2c1cc(C)cc(C2(O)CCOC2)n1]

To a mixture of N-(1′,2′-dihydrospiro[cyclopropane-1,3′-pyrrolo[3,2-c]pyridin]-6′-yl)acetamide (Preparation 7, 50 mg, 246 μmol), 3-(6-bromo-4-methyl-2-pyridyl)tetrahydrofuran-3-ol (127 mg, 492 μmol) and Cs₂CO₃ (160 mg, 492 μmol) in dioxane (2 mL) was added BrettPhos Pd G3 (11 mg, 12.3 μmol). The reaction mixture was heated at 100° C. for 1 h under N₂. The mixture was diluted with EtOAc and washed with water. The organic layer was separated, dried and concentrated under reduced pressure. The crude was purified by chromatography on silica gel (0-100% EtOAc-EtOH 3:1 with 2% NH₄OH in heptane) to give N-(1′-(6-(3-hydroxytetrahydrofuran-3-yl)-4-methylpyridin-2-yl)-1′,2′-dihydrospiro[cyclopropane-1,3′-pyrrolo[3,2-c]pyridin]-6′-yl)acetamide (52 mg, 56% yield) as a white powder. LCMS m/z=381.2 [M+H]⁺. ¹H NMR: (500 MHz, DMSO-d₆) δ: ppm 10.14 (s, 1H), 8.72 (s, 1H), 7.56 (s, 1H), 7.19 (s, 1H), 6.58 (s, 1H), 5.40 (s, 1H), 4.20-4.10 (m, 2H), 4.10-4.00 (m, 1H), 4.00-3.90 (m, 2H), 3.80-3.70 (m, 1H), 2.83 (td, 1H, J=9.1, 12.4 Hz), 2.33 (s, 3H), 2.04 (s, 3H), 2.02-1.97 (m, 1H), 1.20-1.10 (m, 4H).

### Example 96

### N-(1′-(6-(3-methoxytetrahydrofuran-3-yl)-4-methylpyridin-2-yl)-1′,2′-dihydrospiro[cyclopropane-1,3′-pyrrolo[3,2-c]pyridin]-6′-yl)acetamide

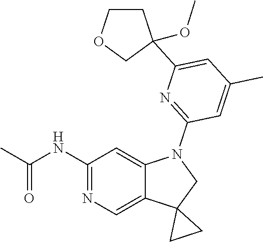 [SMILES: COC1(c2cc(C)cc(N3CC4(CC4)c4cnc(NC(C)=O)cc43)n2)CCOC1]

To a mixture of N-(1′,2′-dihydrospiro[cyclopropane-1,3′-pyrrolo[3,2-c]pyridin]-6′-yl)acetamide (Preparation 7, 50 mg, 246 μmol), 2-bromo-6-(3-methoxytetrahydrofuran-3-yl)-4-methyl-pyridine (Preparation 56, 134 mg, 492 μmol) and Cs₂CO₃ (176 mg, 541 μmol) in dioxane (2.50 mL) was added BrettPhos Pd G3 (11 mg, 12.3 μmol). The mixture was purged with N₂, then heated at 90° C. for 30 min. The reaction mixture was diluted with EtOAc, washed with water, the organic layer was separated, dried and concentrated. The crude was purified by chromatography on silica gel (0-100% EtOAc-EtOH 3:1 with 2% NH₄OH in heptane) to give N-(1′-(6-(3-methoxytetrahydrofuran-3-yl)-4-methylpyridin-2-yl)-1′,2′-dihydrospiro[cyclopropane-1,3′-pyrrolo[3,2-c]pyridin]-6′-yl)acetamide (52 mg, 53% yield) as a white powder. LCMS m/z=395.3 [M+H]^{+ 1}H NMR (400 MHz, CDCl₃) δ 8.80-8.60 (m, 1H), 8.30-8.00 (m, 1H), 7.41 (s, 1H), 7.01 (s, 1H), 6.58 (s, 1H), 4.30-4.00 (m, 6H), 3.27 (s, 3H), 2.83 (td, 1H, J=8.6, 13.3 Hz), 2.50-2.40 (m, 1H), 2.40 (s, 3H), 2.19 (s, 3H), 1.20-1.10 (m, 4H).

### Example 97

### N-(1′-(6-methyl-2-(1-(methylsulfonyl)ethyl)pyrimidin-4-yl)-1′,2′-dihydrospiro[cyclopropane-1,3′-pyrrolo[3,2-c]pyridin]-6′-yl)acetamide

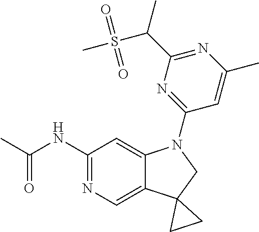 [SMILES: CC(=O)Nc1cc2c(cn1)C1(CC1)CN2c1cc(C)nc(C(C)S(C)(=O)=O)n1]

N-(1′-(6-methyl-2-(1-(methylsulfonyl)ethyl)pyrimidin-4-yl)-1′,2′-dihydrospiro[cyclopropane-1,3′-pyrrolo[3,2-c]pyridin]-6′-yl)acetamide was obtained, 7 mg, 7% yield as a white powder, from N-(1′,2′-dihydrospiro[cyclopropane-1,3′-pyrrolo[3,2-c]pyridin]-6′-yl)acetamide (Preparation 7) and 4-chloro-6-methyl-2-(1-methylsulfonylethyl)pyrimidine following the procedure described in Example 96. LCMS m/z=402.2 [M+H]^{+ 1}H NMR (400 MHz, DMSO-d₆,) δ 10.35 (s, 1H), 9.11 (s, 1H), 7.69 (s, 1H), 6.69 (s, 1H), 4.39 (q, 1H, J=7.3 Hz), 4.20-4.00 (m, 2H), 3.10 (s, 3H), 2.50-2.40 (m, 3H), 2.10-2.00 (m, 3H), 1.85 (d, 3H, J=7.3 Hz), 1.20-1.10 (m, 4H).

### Example 98

### N-(1′-(2-(1,1-dioxidotetrahydrothiophen-3-yl)-6-methylpyrimidin-4-yl)-1′,2′-dihydrospiro[cyclopropane-1,3′-pyrrolo[3,2-c]pyridin]-6′-yl)acetamide

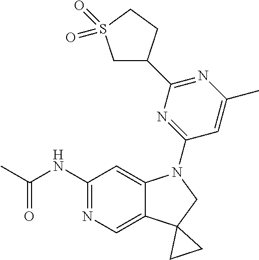 [SMILES: CC(=O)Nc1cc2c(cn1)C1(CC1)CN2c1cc(C)nc(C2CCS(=O)(=O)C2)n1]

N-(1′-(2-(1,1-dioxidotetrahydrothiophen-3-yl)-6-methylpyrimidin-4-yl)-1′,2′-dihydrospiro[cyclopropane-1,3′-pyrrolo[3,2-c]pyridin]-6′-yl)acetamide was prepared in an analogous manner of that in Example 96 from N-(1′,2′-dihydrospiro[cyclopropane-1,3′-pyrrolo[3,2-c]pyridin]-6′-yl)acetamide (Preparation 7) and 3-(4-chloro-6-methylpyrimidin-2-yl)tetrahydrothiophene 1,1-dioxide. LCMS m/z=414.2 [M+H]^{+ 1}H NMR (400 MHz, CDCl₃) δ 9.04 (s, 1H), 8.06 (br s, 1H), 7.51 (s, 1H), 6.41 (s, 1H), 4.08 (s, 2H), 4.00-3.90 (m, 1H), 3.80-3.70 (m, 1H), 3.60-3.50 (m, 1H), 3.46 (ddd, 1H, J=5.1, 8.2, 13.2 Hz), 3.21 (td, 1H, J=8.3, 13.1 Hz), 2.90-2.70 (m, 2H), 2.47 (s, 3H), 2.21 (s, 3H), 1.23-1.11 (m, 4H).

### Example 99

### N-(1′-(4-(2-methoxyethoxy)pyrimidin-2-yl)-1′,2′-dihydrospiro[cyclopropane-1,3′-pyrrolo[3,2-c]pyridin]-6′-yl)acetamide

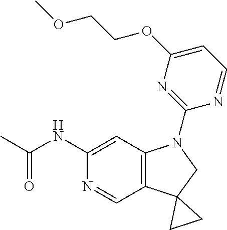 [SMILES: COCCOc1ccnc(N2CC3(CC3)c3cnc(NC(C)=O)cc32)n1]

N-(1′-(4-(2-methoxyethoxy)pyrimidin-2-yl)-1′,2′-dihydrospiro[cyclopropane-1,3′-pyrrolo[3,2-c]pyridin]-6′-yl)acetamide was obtained, 25 mg, 28% yield as a white powder, from N-(1′,2′-dihydrospiro[cyclopropane-1,3′-pyrrolo[3,2-c]pyridin]-6′-yl)acetamide (Preparation 7) and 2-chloro-4-(2-methoxyethoxy)pyrimidine following the procedure described in Example 96. LCMS m/z=356.1 [M+H]⁺ ¹H NMR (400 MHz, CDCl₃) δ 9.02 (s, 1H), 8.40-8.10 (m, 2H), 7.45 (s, 1H), 6.39 (d, 1H, J=5.8 Hz), 4.90-4.70 (m, 2H), 4.27 (s, 2H), 3.90-3.80 (m, 2H), 3.47 (s, 3H), 2.18 (s, 3H), 1.14 (br d, 4H, J=2.8 Hz).

### Example 100

### N-(1′-(6-methyl-2-(1H-pyrazol-1-yl)pyrimidin-4-yl)-1′,2′-dihydrospiro[cyclopropane-1,3′-pyrrolo[3,2-c]pyridin]-6′-yl)acetamide

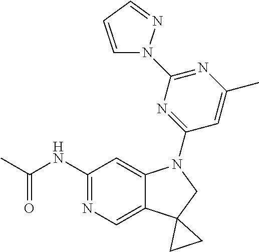 [SMILES: CC(=O)Nc1cc2c(cn1)C1(CC1)CN2c1cc(C)nc(-n2cccn2)n1]

N-(1′-(6-methyl-2-(1H-pyrazol-1-yl)pyrimidin-4-yl)-1′,2′-dihydrospiro[cyclopropane-1,3′-pyrrolo[3,2-c]pyridin]-6′-yl)acetamide was obtained, 12 mg, 13.5% yield as a white powder, from N-(1′,2′-dihydrospiro[cyclopropane-1,3′-pyrrolo[3,2-c]pyridin]-6′-yl)acetamide (Preparation 7) and 4-chloro-6-methyl-2-pyrazol-1-yl-pyrimidine following the procedure described in Example 96. LCMS m/z=362.2 [M+H]⁺. ¹H NMR: (400 MHz, CDCl₃) δ: ppm 9.17 (br s, 2H), 8.18 (br s, 1H), 7.90-7.80 (m, 1H), 7.51 (s, 1H), 6.59 (dd, 1H, J=1.6, 2.6 Hz), 6.38 (s, 1H), 4.15 (s, 2H), 2.60 (s, 3H), 2.24 (s, 3H), 1.23-1.20 (m, 4H).

### Example 101

### N-(1′-(6-(1,1-difluoroethyl)pyrazin-2-yl)-1′,2′-dihydrospiro[cyclopropane-1,3′-pyrrolo[3,2-c]pyridin]-6′-yl)acetamide

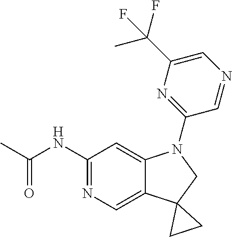 [SMILES: CC(=O)Nc1cc2c(cn1)C1(CC1)CN2c1cncc(C(C)(F)F)n1]

To a mixture of N-(1′,2′-dihydrospiro[cyclopropane-1,3′-pyrrolo[3,2-c]pyridin]-6′-yl)acetamide (Preparation 7, 50 mg, 246 μmol), 2-chloro-6-(1,1-difluoroethyl)pyrazine (88 mg, 492 μmol), NaOtBu (71 mg, 738 μmol), Xantphos Pd G3 (23 mg, 24.6 μmol) and Xantphos (14 mg, 24.6 μmol) in dioxane (2.5 mL) was purged with N₂, then heated at 90° C. for 2 h. The reaction mixture was diluted with EtOAc, washed with water, the organic layer was separated, dried and concentrated. The crude was purified by chromatography on silica gel (0-100% EtOAc-EtOH 3:1 with 2% NH₄OH in heptane) to give N-(1′-(6-(1,1-difluoroethyl)pyrazin-2-yl)-1′,2′-dihydrospiro[cyclopropane-1,3′-pyrrolo[3,2-c]pyridin]-6′-yl)acetamide (13 mg, 15% yield) as a white powder. LCMS m/z=346.1 [M+H]⁺ ¹H NMR (400 MHz, CDCl₃) δ 9.00 (s, 1H), 8.54 (s, 1H), 8.26 (s, 1H), 8.22-8.14 (m, 1H), 7.50 (s, 1H), 4.20 (s, 2H), 2.30-2.10 (m, 6H), 1.24-1.16 (m, 4H).

### Example 102

### N-(1′-(2-(7-oxabicyclo[2.2.1]heptan-2-yl)-6-methylpyrimidin-4-yl)-1′,2′-dihydrospiro[cyclopropane-1,3′-pyrrolo[3,2-c]pyridin]-6′-yl)acetamide trifluoroacetate

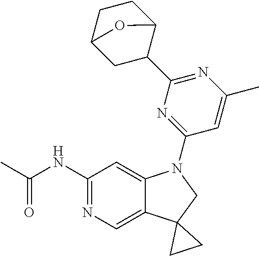 [SMILES: CC(=O)Nc1cc2c(cn1)C1(CC1)CN2c1cc(C)nc(C2CC3CCC2O3)n1]

N-(1′-(2-(7-Oxabicyclo[2.2.1]heptan-2-yl)-6-methylpyrimidin-4-yl)-1′,2′-dihydrospiro[cyclopropane-1,3′-pyrrolo[3,2-c]pyridin]-6′-yl)acetamide was prepared in a similar manner of that described in Example 101 from N-(1′,2′-dihydrospiro[cyclopropane-1,3′-pyrrolo[3,2-c]pyridin]-6′-yl)acetamide (Preparation 7) and 2-(7-oxabicyclo[2.2.1]heptan-2-yl)-4-chloro-6-methylpyrimidine, except the compound was further purified by HPLC, eluting with MeCN: water with 0.1% TFA modifier. LCMS m/z=392.3 [M+H]⁺ ¹H NMR (DMSO-d₆, 600 MHz) δ: 11.00-10.30 (m, 1H), 8.87 (br s, 1H), 7.80-7.60 (m, 1H), 6.80-6.70 (m, 1H), 4.96 (br s, 1H), 4.69 (br t, 1H, J=4.4 Hz), 4.19 (s, 2H), 4.17 (br s, 1H), 2.44 (s, 3H), 2.13 (s, 3H), 2.00-1.90 (m, 2H), 1.80-1.70 (m, 2H), 1.70-1.50 (m, 2H), 1.21 (br d, 4H, J=12.8 Hz).

### Example 103

### N-(1′-(6-methyl-2-(tetrahydrofuran-3-yl)pyrimidin-4-yl)-1′,2′-dihydrospiro[cyclopropane-1,3′-pyrrolo[3,2-c]pyridin]-6′-yl)acetamide

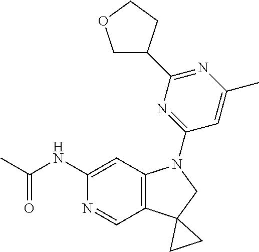 [SMILES: CC(=O)Nc1cc2c(cn1)C1(CC1)CN2c1cc(C)nc(C2CCOC2)n1]

A solution of N-(1′,2′-dihydrospiro[cyclopropane-1,3′-pyrrolo[3,2-c]pyridin]-6′-yl)acetamide (Preparation 7, 100 mg, 492 μmol), 4-chloro-6-methyl-2-(tetrahydrofuran-3-yl)pyrimidine (195 mg, 984 μmol), NaOtBu (142 mg, 1.5 mmol), Xantphos (28.5 mg, 49.2 μmol) and Xantphos Pd G3 (49.1 mg, 49.2 μmol) in dioxane (4 mL) was stirred at 90° C. for 1 h under N₂. The mixture was filtered and the filtrate was concentrated under reduced pressure. The residue was purified by chromatography on silica gel (0-100% EtOAc-EtOH 3:1 with 2% NH₄OH in heptane) to give N-(1′-(6-methyl-2-(tetrahydrofuran-3-yl)pyrimidin-4-yl)-1′,2′-dihydrospiro[cyclopropane-1,3′-pyrrolo[3,2-c]pyridin]-6′-yl)acetamide (121 mg, 67% yield) as a white powder. LCMS m/z=366.2 [M+H]⁺. ¹H NMR: (400 MHz, DMSO-d₆) δ: ppm 10.25 (s, 1H), 9.05 (s, 1H), 7.65 (s, 1H), 6.56 (s, 1H), 4.17 (t, 1H, J=8.0 Hz), 4.12-4.06 (m, 2H), 3.93 (dt, 1H, J=5.4, 8.2 Hz), 3.90-3.80 (m, 2H), 3.55 (quin, 1H, J=8.0 Hz), 2.50-2.40 (m, 1H), 2.37 (s, 3H), 2.30-2.20 (m, 1H), 2.07 (s, 3H), 1.20-1.10 (m, 4H).

### Examples 104 to 125

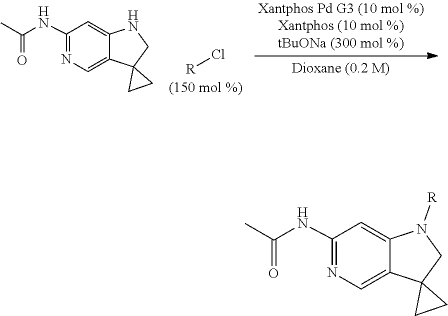

An 8 mL vial was charged with N-(1′,2′-dihydrospiro[cyclopropane-1,3′-pyrrolo[3,2-c]pyridin]-6′-yl)acetamide (Preparation 7, 10.2 mg, 50 μmol), R—Cl (75 μmol), Xantphos Pd G3 (4.7 mg, 5 μmol), Xantphos (2.8 mg, 5 μmol), and NaOtBu (14.4 mg, 150 μmol). Dioxane (1 mL, 0.2 M) was added under an inert atmosphere. The vial was sealed with a Teflon cap, and the reaction solution was heated under reflux at 90° C. for 2 h. The cooled reaction mixture was diluted EtOAc, washed with H₂O (3 mL), and extracted with EtOAc (5 mL, 2×). The combined organic phases were dried over anhydrous MgSO₄ and concentrated in vacuo to give the crude product.

This was purified by HPLC using a Waters XSelect Prep C18 column (5 um OBD 19×100 mm) or Prep C18 (5 um OBD 30×100 mm) or Waters SunFire Prep C18 column (5 um OBD 19×100 mm) under basic or acidic conditions.
Basic conditions (Xselect OBD 19): MeCN, water, with 0.2% NH₄OH modifier
Basic conditions (Xselect OBD 30): MeCN, water, with 0.2% NH₄OH modifier
Acidic conditions (Sunfire OBD 19): MeCN, water, with 0.1% TFA

| Example No | Name, Structure, R—Cl, HPLC, Data |
|---|---|
| 104 | N-(1′-(2-(1-methoxycyclobutyl)-6-methylpyrimidin-4-yl)-1′,2′- |
| | dihydrospiro[cyclopropane-1,3′-pyrrolo[3,2-c]pyridin]-6′-yl)acetamide |
| | 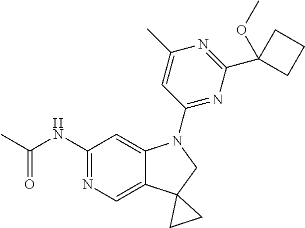 [SMILES: COC1(c2nc(C)cc(N3CC4(CC4)c4cnc(NC(C)=O)cc43)n2)CCC1] |
| | R—Cl: 4-chloro-2-(1-methoxycyclobutyl)-6-methylpyrimidine |
| | HPLC: Basic conditions (Xselect OBD 30): Gradient: 5-55% |
| | 3.7 mg, LCMS m/z = 380.3 [M + H]⁺ |
| 105 | N-(1′-(6-methyl-2-phenylpyrimidin-4-yl)-1′,2′- |
| | dihydrospiro[cyclopropane-1,3′-pyrrolo[3,2-c]pyridin]-6′-yl)acetamide |
| | 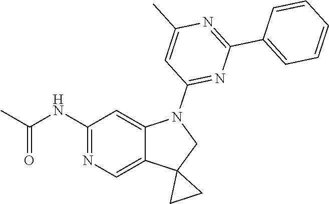 [SMILES: CC(=O)Nc1cc2c(cn1)C1(CC1)CN2c1cc(C)nc(-c2ccccc2)n1] |
| | R—Cl: 4-chloro-6-methyl-2-phenylpyrimidine |
| | HPLC: Basic conditions (Xselect OBD 19): Gradient 5-70% |
| | 1.2 mg, LCMS m/z = 372.2 [M + H]⁺ |
| 106 | N-(1′-(6-methyl-2-(pyridin-3-yl)pyrimidin-4-yl)-1′,2′- |
| | dihydrospiro[cyclopropane-1,3′-pyrrolo[3,2-c]pyridin]-6′-yl)acetamide |
| | 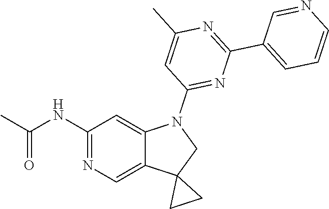 [SMILES: CC(=O)Nc1cc2c(cn1)C1(CC1)CN2c1cc(C)nc(-c2cccnc2)n1] |
| | R—Cl: 4-chloro-6-methyl-2-(pyridin-3-yl)pyrimidine |
| | HPLC: Basic conditions (Xselect OBD 19): Gradient 5-55% |
| | 0.6 mg, LCMS m/z = 373.2 [M + H]⁺ |
| 107 | N-(1′-(6-methyl-2-(pyridin-4-yl)pyrimidin-4-yl)-1′,2′- |
| | dihydrospiro[cyclopropane-1,3′-pyrrolo[3,2-c]pyridin]-6′-yl)acetamide |
| | 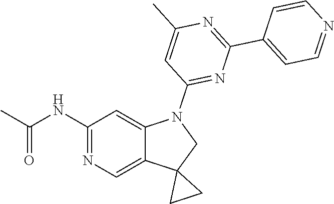 [SMILES: CC(=O)Nc1cc2c(cn1)C1(CC1)CN2c1cc(C)nc(-c2ccncc2)n1] |
| | R—Cl: 4-chloro-6-methyl-2-(pyridin-4-yl)pyrimidine |
| | HPLC: Basic conditions (Xselect OBD 19): Gradient 5-55% |
| | 1.9 mg, LCMS m/z = 373.2 [M + H]⁺ |
| 108 | N-(1′-(6-methyl-[2,2′-bipyrimidin]-4-yl)-1′,2′-dihydrospiro[cyclopropane- |
| | 1,3′-pyrrolo[3,2-c]pyridin]-6′-yl)acetamide |
| | 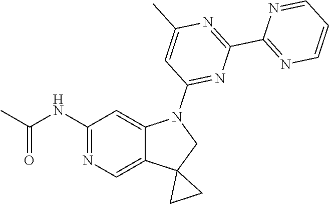 [SMILES: CC(=O)Nc1cc2c(cn1)C1(CC1)CN2c1cc(C)nc(-c2ncccn2)n1] |
| | R—Cl: 4-chloro-6-methyl-2,2′-bipyrimidine |
| | HPLC: Basic conditions (Xselect OBD 19): Gradient 5-40% |
| | 3.5 mg, LCMS m/z = 374.2 [M + H]⁺ |
| 109 | N-(1′-(6-methyl-2-(2-methyloxazol-4-yl)pyrimidin-4-yl)-1′,2′- |
| | dihydrospiro[cyclopropane-1,3′-pyrrolo[3,2-c]pyridin]-6′-yl)acetamide |
| | 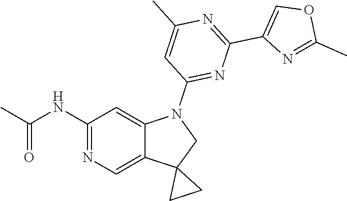 [SMILES: CC(=O)Nc1cc2c(cn1)C1(CC1)CN2c1cc(C)nc(-c2coc(C)n2)n1] |
| | R—Cl: 4-(4-chloro-6-methylpyrimidin-2-yl)-2-methyloxazole |
| | HPLC: Basic conditions (Xselect OBD 19): Gradient 5-50% |
| | 0.8 mg, LCMS m/z = 377.2 [M + H]⁺ |
| 110 | N-(1′-(2-(3,5-dimethyl-1H-pyrazol-1-yl)-6-methylpyrimidin-4-yl)-1′,2′- |
| | dihydrospiro[cyclopropane-1,3′-pyrrolo[3,2-c]pyridin]-6′-yl)acetamide |
| | 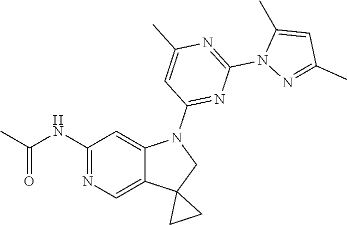 [SMILES: CC(=O)Nc1cc2c(cn1)C1(CC1)CN2c1cc(C)nc(-n2nc(C)cc2C)n1] |
| | R—Cl: 4-chloro-2-(3,5-dimethyl-1H-pyrazol-1-yl)-6-methylpyrimidine |
| | HPLC: Basic conditions (Xselect OBD 19): Gradient 10-70% |
| | 5.9 mg, LCMS m/z = 390.3 [M + H]⁺; |
| 111 | N-(1′-(2-(3-fluorophenyl)-6-methylpyrimidin-4-yl)-1′,2′- |
| | dihydrospiro[cyclopropane-1,3′-pyrrolo[3,2-c]pyridin]-6′-yl)acetamide |
| | 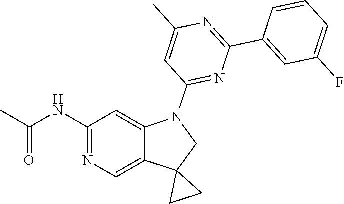 [SMILES: CC(=O)Nc1cc2c(cn1)C1(CC1)CN2c1cc(C)nc(-c2cccc(F)c2)n1] |
| | R—Cl: 4-chloro-2-(3-fluorophenyl)-6-methylpyrimidine |
| | HPLC: Basic conditions (Xselect OBD 19): Gradient 5-70% |
| | 0.5 mg, LCMS m/z = 390.2 [M + H]⁺; |
| 112 | N-(1′-(6-methyl-2-(pyridin-2-yl)pyrimidin-4-yl)-1′,2′- |
| | dihydrospiro[cyclopropane-1,3′-pyrrolo[3,2-c]pyridin]-6′-yl)acetamide |
| | 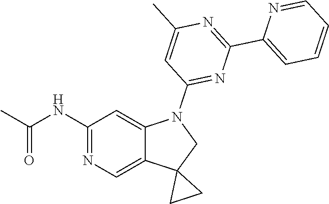 [SMILES: CC(=O)Nc1cc2c(cn1)C1(CC1)CN2c1cc(C)nc(-c2ccccn2)n1] |
| | R—Cl: 4-chloro-6-methyl-2-(pyridin-2-yl)pyrimidine |
| | HPLC: Basic conditions (Xselect OBD 19): Gradient 5-50% |
| | 1.9 mg, LCMS m/z = 373.2 [M + H]⁺ |
| 113 | N-(1′-(6-methyl-2-(piperidin-1-yl)pyrimidin-4-yl)-1′,2′- |
| | dihydrospiro[cyclopropane-1,3′-pyrrolo[3,2-c]pyridin]-6′-yl)acetamide |
| | 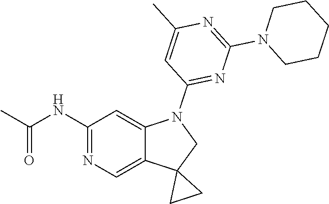 [SMILES: CC(=O)Nc1cc2c(cn1)C1(CC1)CN2c1cc(C)nc(N2CCCCC2)n1] |
| | RCl: 4-chloro-6-methyl-2-(piperidin-1-yl)pyrimidine |
| | HPLC: Basic conditions (Xselect OBD 19): Gradient 10-70% |
| | 2.9 mg, LCMS m/z = 379.3 [M + H]⁺ |
| 114 | N-(1′-(6-methyl-2-(pyrrolidin-1-yl)pyrimidin-4-yl)-1′,2′- |
| | dihydrospiro[cyclopropane-1,3′-pyrrolo[3,2-c]pyridin]-6′-yl)acetamide |
| | 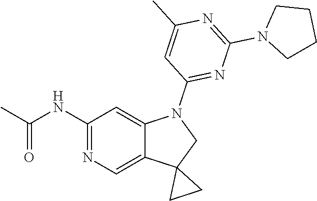 [SMILES: CC(=O)Nc1cc2c(cn1)C1(CC1)CN2c1cc(C)nc(N2CCCC2)n1] |
| | RCl: 4-chloro-6-methyl-2-(pyrrolidin-1-yl)pyrimidine |
| | HPLC: Basic conditions (Xselect OBD 19): Gradient 5-60% |
| | 2.5 mg, LCMS m/z = 365.3 [M + H]⁺ |
| 115 | N-(1′-(2-(isopropyl(methyl)amino)-6-methylpyrimidin-4-yl)-1′,2′- |
| | dihydrospiro[cyclopropane-1,3′-pyrrolo[3,2-c]pyridin]-6′-yl)acetamide |
| | 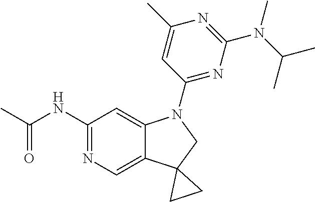 [SMILES: CC(=O)Nc1cc2c(cn1)C1(CC1)CN2c1cc(C)nc(N(C)C(C)C)n1] |
| | RCl: 4-chloro-N-isopropyl-N,6-dimethylpyrimidin-2-amine |
| | HPLC: Basic conditions (Xselect OBD 19): Gradient 10-70% |
| | 4.0 mg, LCMS m/z = 367.3 [M + H]⁺ |
| 116 | N-(1′-(2-(dimethylamino)-6-methylpyrimidin-4-yl)-1′,2′- |
| | dihydrospiro[cyclopropane-1,3′-pyrrolo[3,2-c]pyridin]-6′-yl)acetamide |
| | 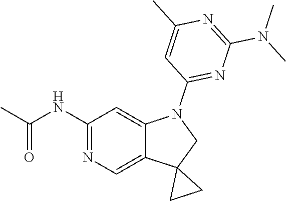 [SMILES: CC(=O)Nc1cc2c(cn1)C1(CC1)CN2c1cc(C)nc(N(C)C)n1] |
| | RCl: 4-chloro-N,N,6-trimethylpyrimidin-2-amine |
| | HPLC: Basic conditions (Xselect OBD 19): Gradient 5-60% |
| | LCMS m/z = 339.2 [M + H]⁺ |
| 117 | N-(1′-(6-methyl-2-(1-methylpiperidin-3-yl)pyrimidin-4-yl)-1′,2′- |
| | dihydrospiro[cyclopropane-1,3′-pyrrolo[3,2-c]pyridin]-6′-yl)acetamide |
| | 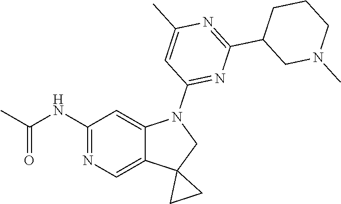 [SMILES: CC(=O)Nc1cc2c(cn1)C1(CC1)CN2c1cc(C)nc(C2CCCN(C)C2)n1] |
| | R—Cl: 4-chloro-6-methyl-2-(1-methylpiperidin-3-yl)pyrimidine |
| | HPLC: Basic conditions (Xselect OBD 19): Gradient 5-55% |
| | 6.9 mg, LCMS m/z = 393.3 [M + H]⁺ |
| 118 | N-(1′-(6-methyl-2-(1-methylpiperidin-2-yl)pyrimidin-4-yl)-1′,2′- |
| | dihydrospiro[cyclopropane-1,3′-pyrrolo[3,2-c]pyridin]-6′-yl)acetamide |
| | 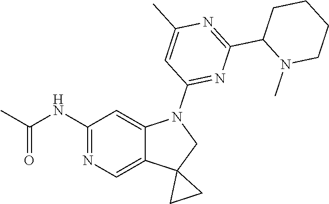 [SMILES: CC(=O)Nc1cc2c(cn1)C1(CC1)CN2c1cc(C)nc(C2CCCCN2C)n1] |
| | RCl: 4-chloro-6-methyl-2-(1-methylpiperidin-2-yl)pyrimidine |
| | HPLC: Basic conditions (Xselect OBD 19): Gradient 5-55% |
| | 7.3 mg, LCMS m/z = 393.3 [M + H]⁺ |
| 119 | N-(1′-(6-methyl-2-(1-(methylsulfonyl)piperidin-3-yl)pyrimidin-4-yl)- |
| | 1′,2′-dihydrospiro[cyclopropane-1,3′-pyrrolo[3,2-c]pyridin]-6′- |
| | yl)acetamide |
| | 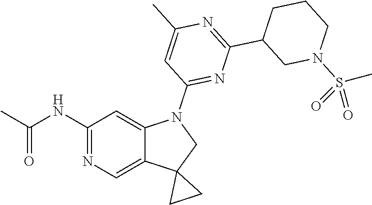 [SMILES: CC(=O)Nc1cc2c(cn1)C1(CC1)CN2c1cc(C)nc(C2CCCN(S(C)(=O)=O)C2)n1] |
| | R—Cl: 4-chloro-6-methyl-2-(1-(methylsulfonyl)piperidin-3-yl)pyrimidine |
| | HPLC: Basic conditions (Xselect OBD 19): Gradient 5-55% |
| | 1.4 mg, LCMS m/z = 457.3 [M + H]⁺ |
| 120 | N-(1′-(2-(1-acetylpiperidin-4-yl)-6-methylpyrimidin-4-yl)-1′,2′- |
| | dihydrospiro[cyclopropane-1,3′-pyrrolo[3,2-c]pyridin]-6′-yl)acetamide |
| | 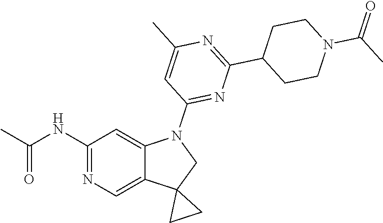 [SMILES: CC(=O)Nc1cc2c(cn1)C1(CC1)CN2c1cc(C)nc(C2CCN(C(C)=O)CC2)n1] |
| | R—Cl: 1-(4-(4-chloro-6-methylpyrimidin-2-yl)piperidin-1-yl)ethan-1-one |
| | HPLC: Basic conditions (Xselect OBD 19): Gradient 5-50% |
| | 4.5 mg, LCMS m/z = 421.3 [M + H]⁺ |
| 121 | N-(1′-(2-(1-isopropylpiperidin-4-yl)-6-methylpyrimidin-4-yl)-1′,2′- |
| | dihydrospiro[cyclopropane-1,3′-pyrrolo[3,2-c]pyridin]-6′-yl)acetamide |
| | 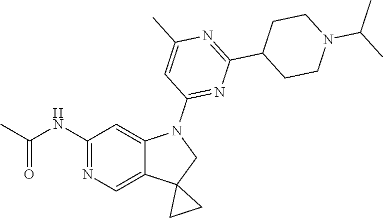 [SMILES: CC(=O)Nc1cc2c(cn1)C1(CC1)CN2c1cc(C)nc(C2CCN(C(C)C)CC2)n1] |
| | R—Cl: 4-chloro-2-(1-isopropylpiperidin-4-yl)-6-methylpyrimidine |
| | HPLC: Acidic conditions (Sunfire OBD 19): Gradient 5-35% |
| | 1.0 mg, LCMS m/z = 421.3 [M + H]⁺ |
| 122 | N-(1′-(6-methyl-2-(1-(methylsulfonyl)piperidin-4-yl)pyrimidin-4-yl)- |
| | 1′,2′-dihydrospiro[cyclopropane-1,3′-pyrrolo[3,2-c]pyridin]-6′- |
| | yl)acetamide |
| | 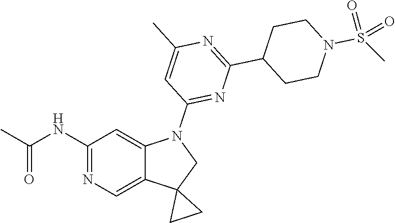 [SMILES: CC(=O)Nc1cc2c(cn1)C1(CC1)CN2c1cc(C)nc(C2CCN(S(C)(=O)=O)CC2)n1] |
| | R—Cl: 4-chloro-6-methyl-2-(1-(methylsulfonyl)piperidin-4-yl)pyrimidine |
| | HPLC: Basic conditions (Xselect OBD 19): Gradient 5-50% |
| | 9.6 mg, LCMS m/z = 457.3 [M + H]⁺ |
| 123 | N-(1′-(6-methyl-2-(2-methylcyclopropyl)pyrimidin-4-yl)-1′,2′- |
| | dihydrospiro[cyclopropane-1,3′-pyrrolo[3,2-c]pyridin]-6′-yl)acetamide |
| | 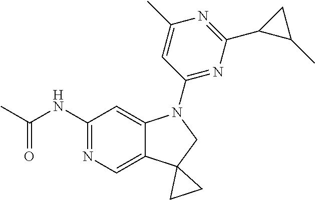 [SMILES: CC(=O)Nc1cc2c(cn1)C1(CC1)CN2c1cc(C)nc(C2CC2C)n1] |
| | R—Cl: 4-chloro-6-methyl-2-(2-methylcyclopropyl)pyrimidine |
| | HPLC: Basic conditions (Xselect OBD 30): gradient: 5-60% |
| | 6.0 mg, LCMS m/z = 350.2 [M + H]⁺ |
| 124 | N-(1′-(2-(2,2-dimethylcyclopropyl)-6-methylpyrimidin-4-yl)-1′,2′- |
| | dihydrospiro[cyclopropane-1,3′-pyrrolo[3,2-c]pyridin]-6′-yl)acetamide |
| | 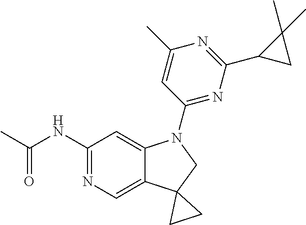 [SMILES: CC(=O)Nc1cc2c(cn1)C1(CC1)CN2c1cc(C)nc(C2CC2(C)C)n1] |
| | R—Cl: 4-chloro-2-(2,2-dimethylcyclopropyl)-6-methylpyrimidine |
| | HPLC: Basic conditions (Xselect OBD 30): gradient: 5-60% 1.7 mg, |
| | LCMS m/z = 364.3 [M + H]⁺ |
| 125 | N-(1′-(2-(3,3-difluorocyclopentyl)-6-methylpyrimidin-4-yl)-1′,2′- |
| | dihydrospiro[cyclopropane-1,3′-pyrrolo[3,2-c]pyridin]-6′-yl)acetamide |
| | 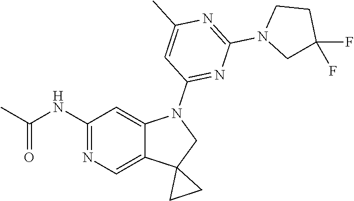 [SMILES: CC(=O)Nc1cc2c(cn1)C1(CC1)CN2c1cc(C)nc(N2CCC(F)(F)C2)n1] |
| | R—Cl: 4-chloro-2-(3,3-difluorocyclopentyl)-6-methylpyrimidine |
| | HPLC: Basic conditions (Xselect OBD 30): gradient: 5-60% |
| | 6.8 mg, LCMS m/z = 400.3 [M + H]⁺ |

### Example 126

### N-(1′-(2-(hydroxymethyl)-6-methylpyrimidin-4-yl)-1′,2′-dihydrospiro[cyclopropane-1,3′-pyrrolo[3,2-c]pyridin]-6′-yl)acetamide

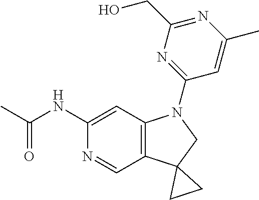 [SMILES: CC(=O)Nc1cc2c(cn1)C1(CC1)CN2c1cc(C)nc(CO)n1]

A mixture of N-(1′,2′-dihydrospiro[cyclopropane-1,3′-pyrrolo[3,2-c]pyridin]-6′-yl)acetamide (Preparation 7, 50 mg, 246 μmol), (4-chloro-6-methyl-pyrimidin-2-yl)methanol (78 mg, 492 μmol), Cs₂CO₃ (160 mg, 492 μmol) and RuPhos Pd G3 (10.3 mg, 12.3 μmol) in dioxane (2 mL) was heated at 90° C. for 30 min under N₂. The reaction mixture was diluted with EtOAc and washed with water. The organic layer was separated, dried and concentrated under reduced pressure. The crude was purified by chromatography on silica gel (10-100% EtOAc-EtOH 3:1 with 2% NH₄OH in heptane) to give N-(1′-(2-(hydroxymethyl)-6-methylpyrimidin-4-yl)-1′,2′-dihydrospiro[cyclopropane-1,3′-pyrrolo[3,2-c]pyridin]-6′-yl)acetamide (37 mg, 46% yield) as a white powder. LCMS m/z=326.1 [M+H]⁺. ¹H NMR: (400 MHz, CDCl₃) δ: ppm 9.40-9.20 (m, 1H), 7.46 (s, 1H), 6.40 (s, 1H), 4.84 (s, 2H), 4.12 (s, 2H), 2.50 (s, 3H), 2.25 (s, 3H), 1.22-1.20 (m, 4H).

### Example 127

### N-(1′-(2-(1,1-difluoroethyl)-5-fluoro-6-methylpyrimidin-4-yl)-1′,2′-dihydrospiro[cyclopropane-1,3′-pyrrolo[3,2-c]pyridin]-6′-yl)acetamide

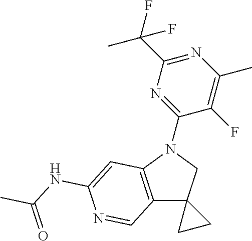 [SMILES: CC(=O)Nc1cc2c(cn1)C1(CC1)CN2c1nc(C(C)(F)F)nc(C)c1F]

To a solution of 4-chloro-2-(1,1-difluoroethyl)-5-fluoro-6-methylpyrimidine (Preparation 36, 100 mg, 0.475 mmol) and N-(1′,2′-dihydrospiro[cyclopropane-1,3′-pyrrolo[3,2-c]pyridin]-6′-yl)acetamide (Preparation 7, 96.5 mg, 0.475 mmol) in DMF (3 mL) was added Cs₂CO₃ (464 mg, 1.42 mmol) at 25° C. The reaction mixture was stirred at 70° C. for 16 h. The mixture was concentrated under reduced pressure to give a residue which was purified by prep-HPLC (Column: Welch Xtimate C18 150*25 mm*5 um; Condition: water (NH₄HCO₃)-MeCN; Begin B: 28; End B: 58; Gradient Time (min): 11; Flow Rate (ml/min): 25; to give N-(1′-(2-(1,1-difluoroethyl)-5-fluoro-6-methylpyrimidin-4-yl)-1′,2′-dihydrospiro[cyclopropane-1,3′-pyrrolo[3,2-c]pyridin]-6′-yl)acetamide (12.0 mg, 6.7% yield) as a white solid. LCMS m/z=378.1 [M+H]⁺. ¹H NMR: (400 MHz, CDCl₃) δ: ppm 8.80 (s, 1H), 8.15 (s, 1H), 7.52 (s, 1H), 4.37 (d, J=4.8 Hz, 3H), 2.55 (d, J=3.2 Hz, 3H), 2.20-2.10 (m, 6H), 1.16-1.06 (m, 4H).

### Example 128

### N-(1′-(2-(1,1-difluoroethyl)-5-fluoropyrimidin-4-yl)-1′,2′-dihydrospiro[cyclopropane-1,3′-pyrrolo[3,2-c]pyridin]-6′-yl)acetamide

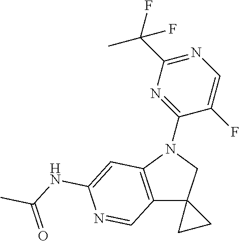 [SMILES: CC(=O)Nc1cc2c(cn1)C1(CC1)CN2c1nc(C(C)(F)F)ncc1F]

To a solution of N-(1′,2′-dihydrospiro[cyclopropane-1,3′-pyrrolo[3,2-c]pyridin]-6′-yl)acetamide (Preparation 7, 100 mg, 0.492 mmol) in DMF (3 mL) was added 4-chloro-2-(1,1-difluoroethyl)-5-fluoropyrimidine (Preparation 47, 116 mg, 0.590 mmol) and Cs₂CO₃ (481 mg, 1.48 mmol). The reaction mixture was stirred at 70° C. for 3 h. The mixture was concentrated and purified by prep-HPLC (Welch Xtimate C18 150*25 mm*5 um; Condition: water (NH₄HCO₃)-MeCN; Begin B: 26; End B: 55; Gradient Time (min): 11; Flow Rate (ml/min): 25) to give N-(1′-(2-(1,1-difluoroethyl)-5-fluoropyrimidin-4-yl)-1′,2′-dihydrospiro[cyclopropane-1,3′-pyrrolo[3,2-c]pyridin]-6′-yl)acetamide (69.8 mg, 39.0% yield) as a white solid. LCMS m/z=364.1 [M+H]⁺. ¹H NMR: (500 MHz, CDCl₃) δ: ppm 8.91 (s, 1H), 8.44 (d, J=5.5 Hz, 1H), 8.16 (s, 1H), 7.55 (s, 1H), 4.40-4.39 (m, 2H), 2.21-2.14 (m, 6H), 1.17-1.16 (m, 4H).

### Example 129

### N-(1′-(2-(2-methoxypropan-2-yl)-6-methylpyrimidin-4-yl)-1′,2′-dihydrospiro[cyclopropane-1,3′-pyrrolo[3,2-c]pyridin]-6′-yl)acetamide

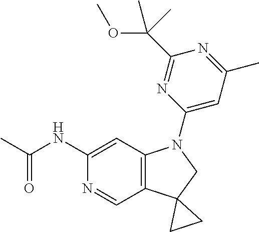 [SMILES: COC(C)(C)c1nc(C)cc(N2CC3(CC3)c3cnc(NC(C)=O)cc32)n1]

A mixture of N-(1′,2′-dihydrospiro[cyclopropane-1,3′-pyrrolo[3,2-c]pyridin]-6′-yl)acetamide (Preparation 7, 50 mg, 246 μmol), KOtBu (55.2 mg, 492 μmol) and 4-chloro-2-(2-methoxypropan-2-yl)-6-methylpyrimidine (74.0 mg, 369 μmol) in DMSO (2 ml) was stirred at rt for 1 h. The mixture was diluted with EtOAc and washed with water. The organic layer was separated, dried and concentrated under reduced pressure. The crude was purified by chromatography on silica gel (0-100% EtOAc-EtOH 3:1 with 2% NH₄OH in heptane) to give N-(1′-(2-(2-methoxypropan-2-yl)-6-methylpyrimidin-4-yl)-1′,2′-dihydrospiro[cyclopropane-1,3′-pyrrolo[3,2-c]pyridin]-6′-yl)acetamide (28 mg, 31% yield) as a white powder. LCMS m/z=368.3 [M+H]⁺. ¹H NMR: (500 MHz,) δ: ppm 9.00-8.80 (m, 1H), 7.58 (s, 1H), 6.61 (s, 1H), 4.13 (s, 2H), 3.20 (s, 3H), 2.49 (s, 3H), 2.17 (s, 3H), 1.68 (s, 6H), 1.21 (br d, 4H, J=5.2 Hz).

### Examples 130 to 132

The compounds in the following table were prepared from N-(1′,2′-dihydrospiro[cyclopropane-1,3′-pyrrolo[3,2-c]pyridin]-6′-yl)acetamide (Preparation 7) and the appropriate heterocycle following a similar procedure to that described in Example 129.

| Example No | Name, Structure, Starting material (SM), Data |
|---|---|
| 130 | N-(1′-(2-(1-methoxyethyl)-6-methylpyrimidin-4-yl)-1′,2′- |
| | dihydrospiro[cyclopropane-1,3′-pyrrolo[3,2-c]pyridin]-6′-yl)acetamide |
| | 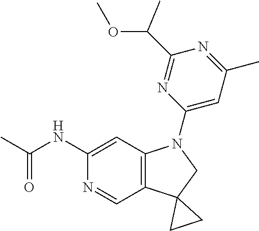 [SMILES: COC(C)c1nc(C)cc(N2CC3(CC3)c3cnc(NC(C)=O)cc32)n1] |
| | SM: 4-chloro-2-(1-methoxyethyl)-6-methylpyrimidine |
| | 7 mg, LCMS m/z = 354.2 [M + H]⁺. ¹H NMR: (500 MHz, MeOH-d₄) δ: ppm |
| | 9.11 (br s, 1H), 7.58 (s, 1H), 6.64 (s, 1H), 4.54 (q, 1H, J = 6.6 Hz), 4.13 (s, |
| | 2H), 3.36 (s, 3H), 2.49 (s, 3H), 2.19 (s, 3H), 1.55 (d, 3H, J = 6.6 Hz), 1.21 |
| | (br d, 4H, J = 4.9 Hz). |
| 131 | N-(1′-(2-(1-methoxyethyl)pyrimidin-4-yl)-1′,2′-dihydrospiro[cyclopropane- |
| | 1,3′-pyrrolo[3,2-c]pyridin]-6′-yl)acetamide |
| | 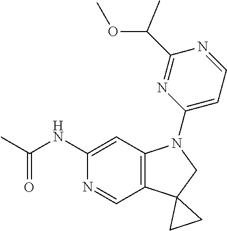 [SMILES: COC(C)c1nccc(N2CC3(CC3)c3cnc(NC(C)=O)cc32)n1] |
| | SM: 4-chloro-2-(1-methoxyethyl)pyrimidine |
| | 16 mg, LCMS m/z = 340.1 [M + H]⁺. ¹H NMR: (500 MHz, MeOH-d₄) δ: |
| | ppm 9.15 (br s, 1H), 8.49 (d, 1H, J = 6.0 Hz), 7.60 (s, 1H), 6.76 (br d, |
| | 1H, J = 6.0 Hz), 4.56 (q, 1H, J = 6.6 Hz), 4.14 (s, 2H), 3.37 (s, 3H), 2.19 (s, |
| | 3H), 1.56 (d, 3H, J = 6.6 Hz), 1.22 (br d, 4H, J = 4.7 Hz). |
| 132 | N-(1′-(2-(2-methoxypropan-2-yl)pyrimidin-4-yl)-1′,2′- |
| | dihydrospiro[cyclopropane-1,3′-pyrrolo[3,2-c]pyridin]-6′-yl)acetamide |
| | 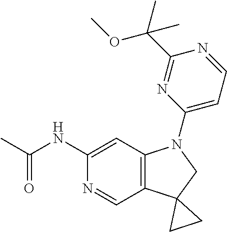 [SMILES: COC(C)(C)c1nccc(N2CC3(CC3)c3cnc(NC(C)=O)cc32)n1] |
| | SM: 4-chloro-2-(2-methoxypropan-2-yl)pyrimidine |
| | 19 mg, LCMS m/z = 354.2 [M + H]⁺. ¹H NMR: (500 MHz, CDCl₃) δ: ppm |
| | 9.10-8.90 (m, 1H), 8.60 (d, 1H, J = 5.8 Hz), 8.13 (br s, 1H), 7.50 (s, 1H), 6.60 |
| | (br d, 1H, J = 5.8 Hz), 4.13 (s, 2H), 3.28 (s, 3H), 2.22 (s, 3H), 1.75 (s, 6H), |
| | 1.19 (br d, 4H, J = 9.0 Hz). |

### Example 133

### N-(1′-(2-(1,1-difluoroethyl)-6-oxo-1,6-dihydropyrimidin-4-yl)-1′,2′-dihydrospiro[cyclopropane-1,3′-pyrrolo[3,2-c]pyridin]-6′-yl)acetamide

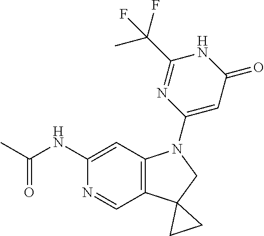 [SMILES: CC(=O)Nc1cc2c(cn1)C1(CC1)CN2c1cc(=O)[nH]c(C(C)(F)F)n1]

A mixture of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 18 mg, 47 μmol) and KOtBu (11 mg, 95 μmol) in DMSO (1 mL) was stirred at rt for 1 h. The mixture was diluted with EtOAc and washed with water (×3). The organic layer was separated, dried and concentrated. The crude was purified by HPLC to give N-(1′-(2-(1,1-difluoroethyl)-6-hydroxypyrimidin-4-yl)-1′,2′-dihydrospiro[cyclopropane-1,3′-pyrrolo[3,2-c]pyridin]-6′-yl)acetamide as a white powder (5 mg, 29% yield). LCMS m/z=362.2 [M+H]⁺.

### Example 134

### N-(1′-(6-methyl-2-oxo-1,2-dihydropyrimidin-4-yl)-1′,2′-dihydrospiro[cyclopropane-1,3′-pyrrolo[3,2-c]pyridin]-6′-yl)acetamide

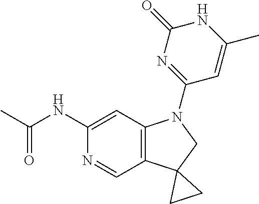 [SMILES: CC(=O)Nc1cc2c(cn1)C1(CC1)CN2c1cc(C)[nH]c(=O)n1]

A mixture of N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 60 mg, 182 μmol) and KOtBu (41 mg, 364 μmol) in DMSO (2 mL) was stirred at rt for 1 h. The mixture was filtered and purified by HPLC to give N-(1′-(6-methyl-2-oxo-1,2-dihydropyrimidin-4-yl)-1′,2′-dihydrospiro[cyclopropane-1,3′-pyrrolo[3,2-c]pyridin]-6′-yl)acetamide (9 mg, 16% yield). LCMS m/z=312.2 [M+H]⁺. ¹H NMR (600 MHz, DMSO-d₆) δ: ppm 11.25 (br s, 1H), 10.60-10.0 (m, 1H), 8.80 (br s, 1H), 7.67 (s, 1H), 5.88 (s, 1H), 4.11 (s, 2H), 2.20-2.10 (m, 3H), 2.10-2.00 (m, 3H), 1.14 (br d, 4H, J=7.8 Hz).

### Example 135

### N-(1′-(4-methyl-6-oxo-1,6-dihydropyrimidin-2-yl)-1′,2′-dihydrospiro[cyclopropane-1,3′-pyrrolo[3,2-c]pyridin]-6′-yl)acetamide

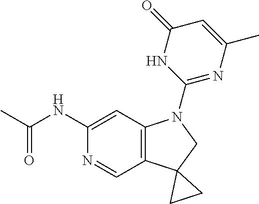 [SMILES: CC(=O)Nc1cc2c(cn1)C1(CC1)CN2c1nc(C)cc(=O)[nH]1]

N-(1′-(4-Methyl-6-oxo-1,6-dihydropyrimidin-2-yl)-1′,2′-dihydrospiro[cyclopropane-1,3′-pyrrolo[3,2-c]pyridin]-6′-yl)acetamide (7.5 mg) was prepared using a similar method to the one described in Example 134 from N-(1′-(4-chloro-6-methylpyrimidin-2-yl)-1′,2′-dihydrospiro[cyclopropane-1,3′-pyrrolo[3,2-c]pyridin]-6′-yl)acetamide (Preparation 154). LCMS m/z=312.0 [M+H]⁺. ¹H NMR (600 MHz, DMSO-d₆) δ: ppm 10.40-10.00 (m, 1H), 9.20-8.80 (m, 1H), 7.56 (s, 1H), 5.97 (br s, 1H), 4.12 (s, 2H), 2.25 (s, 3H), 2.05 (s, 3H), 1.12-1.08 (m, 4H)

### Example 136

### N-(1′-(6-methyl-2-(methylsulfonyl)pyrimidin-4-yl)-1′,2′-dihydrospiro[cyclopropane-1,3′-pyrrolo[3,2-c]pyridin]-6′-yl)acetamide

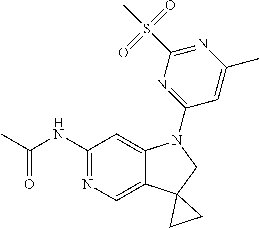 [SMILES: CC(=O)Nc1cc2c(cn1)C1(CC1)CN2c1cc(C)nc(S(C)(=O)=O)n1]

To a mixture of N-(1′-(6-methyl-2-(methylthio)pyrimidin-4-yl)-1′,2′-dihydrospiro[cyclopropane-1,3′-pyrrolo[3,2-c]pyridin]-6′-yl)acetamide (Example 29, 60 mg, 176 μmol) in THF (1.5 mL) and water (1.5 mL) was added trichlororuthenium hydrate (4 mg, 17.6 μmol), followed by IO₄ (75.2 mg, 351 μmol) at 0° C. The reaction mixture was stirred at 0° C. for 30 min. The mixture was diluted with water and the product was extracted with EtOAc (×3). The organic layer was separated, dried and concentrated under reduced pressure. The solid formed was diluted with MeCN and rinsed with cold MeCN to give N-(1′-(6-methyl-2-(methylsulfonyl)pyrimidin-4-yl)-1′,2′-dihydrospiro[cyclopropane-1,3′-pyrrolo[3,2-c]pyridin]-6′-yl)acetamide (42 mg, 64% yield) as a white solid. LCMS m/z=374.0 [M+H]⁺. ¹H NMR: (400 MHz, CDCl₃) δ: ppm 9.00-8.80 (m, 1H), 8.20 (br s, 1H), 7.54 (s, 1H), 6.69 (br s, 1H), 4.17 (s, 2H), 3.55 (s, 3H), 2.62 (s, 3H), 2.22 (s, 3H), 1.23-1.19 (m, 4H).

### Example 137

### N-(1′-(2-(3-methoxypyrrolidin-1-yl)-6-methylpyrimidin-4-yl)-1′,2′-dihydrospiro[cyclopropane-1,3′-pyrrolo[3,2-c]pyridin]-6′-yl)acetamide

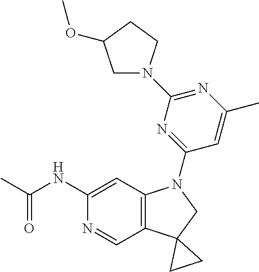 [SMILES: COC1CCN(c2nc(C)cc(N3CC4(CC4)c4cnc(NC(C)=O)cc43)n2)C1]

A mixture of N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 60 mg, 182 μmol), 3-methoxypyrrolidine hydrochloride (125 mg, 910 μmol) and DIPEA (235 mg, 1.82 mmol) in IPA (3 mL) was heated at 110° C. for 2 h. The solvent was removed, the crude was purified by chromatography on silica gel (0-100% EtOAc-EtOH 3:1 with 2% NH₄OH in heptane) to give N-(1′-(2-(3-methoxypyrrolidin-1-yl)-6-methylpyrimidin-4-yl)-1′,2′ -dihydrospiro[cyclopropane-1,3′-pyrrolo[3,2-c]pyridin]-6′-yl)acetamide(47 mg, 65% yield). LCMS m/z=395.4 [M+H]⁺. ¹H NMR: (400 MHz, CDCl₃) δ: ppm 9.06 (br s, 1H), 8.40-8.10 (m, 1H), 7.40 (s, 1H), 5.77 (s, 1H), 4.20-4.10 (m, 1H), 4.05 (s, 2H), 4.00-3.60 (m, 4H), 3.42 (s, 3H), 2.33 (s, 3H), 2.18 (s, 5H), 1.20-1.10 (m, 4H).

### Example 138

### N-(1′-(2-cyclobutoxy-6-methylpyrimidin-4-yl)-1′,2′-dihydrospiro[cyclopropane-1,3′-pyrrolo[3,2-c]pyridin]-6′-yl)acetamide

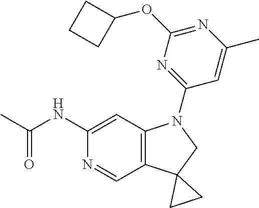 [SMILES: CC(=O)Nc1cc2c(cn1)C1(CC1)CN2c1cc(C)nc(OC2CCC2)n1]

A mixture of N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 50 mg, 152 μmol), cyclobutanol (32.8 mg, 455 μmol) and Cs₂CO₃ (148 mg, 455 μmol) in DMF (2 mL) was heated at 90° C. for 16 h. The mixture was diluted with EtOAc and washed with water (×3). The organic layer was dried and concentrated under reduced pressure. The crude was purified by chromatography on silica gel (0-100% EtOAc-EtOH 3:1 with 2% NH₄OH in heptane) to give N-(1′-(2-cyclobutoxy-6-methylpyrimidin-4-yl)-1′,2′-dihydrospiro[cyclopropane-1,3′-pyrrolo[3,2-c]pyridin]-6′-yl)acetamide (36 mg, 58% yield). LCMS m/z=366.2 [M+H]⁺. ¹H NMR: (400 MHz, CDCl₃) δ: ppm 9.10-8.90 (m, 1H), 8.32 (br s, 1H), 7.46 (s, 1H), 6.18 (s, 1H), 5.54 (t, 1H, J=7.3 Hz), 4.09 (s, 2H), 2.60-2.50 (m, 2H), 2.40 (s, 3H), 2.30-2.20 (m, 5H), 1.90-1.70 (m, 2H), 1.20-1.10 (m, 4H).

### Example 139 to 152

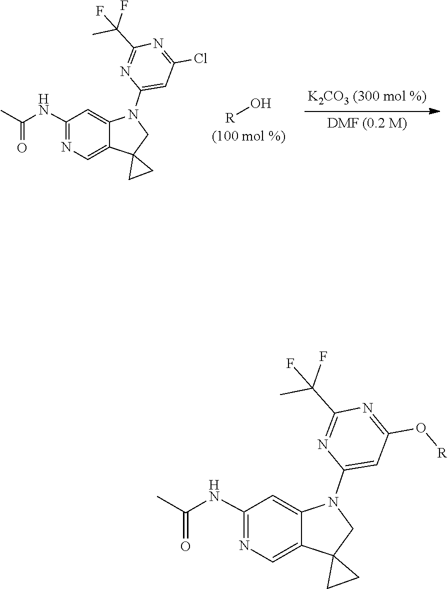

In a 2-dram vial, a solution of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 19.0 mg, 50 μmol), R—OH (50 μmol) and K₂CO₃ (20.7 mg, 150 μmol) in DMF (1 mL, 0.2 M) was sealed with a Teflon cap, and the reaction solution was heated at 50° C. for 16 h. After cooling to rt, the reaction mixture was diluted with EtOAc, washed with H₂ (3 mL), and extracted with EtOAc (5 mL, 2×). The combined organic phases were dried over anhydrous MgSO₄ and concentrated in vacuo to provide the crude product. This was purified by HPLC using a Waters SunFire Prep C18 column (5 um OBD 19×100 mm) under basic or acidic conditions.
Basic conditions (Sunfire OBD 19): water, MeCN, with 0.2% NH₄OH
Acidic conditions (Sunfire OBD 19): water, MeCN, with 0.1% TFA

| Example No | Name, Structure, R—OH, HPLC, Data |
|---|---|
| 139 | N-(1′-(2-(1,1-difluoroethyl)-6-((3-methyloxetan-3-yl)methoxy)pyrimidin-4- |
| | yl)-1′,2′-dihydrospiro[cyclopropane-1,3′-pyrrolo[3,2-c]pyridin]-6′- |
| | yl)acetamide trifluoroacetate |
| | 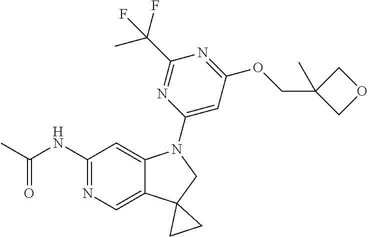 [SMILES: CC(=O)Nc1cc2c(cn1)C1(CC1)CN2c1cc(OCC2(C)COC2)nc(C(C)(F)F)n1] |
| | R—OH: (3-methyloxetan-3-yl)methanol |
| | HPLC: Acidic conditions (Sunfire OBD 19), Gradient 5-50% |
| | 1.1 mg, LCMS m/z = 446.3 [M + H]⁺ |
| 140 | N-(1′-(2-(1,1-difluoroethyl)-6-(2-(dimethylamino)ethoxy)pyrimidin-4-yl)- |
| | 1′,2′-dihydrospiro[cyclopropane-1,3′-pyrrolo[3,2-c]pyridin]-6′-yl)acetamide |
| | trifluoroacetate |
| | 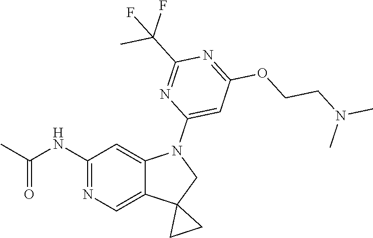 [SMILES: CC(=O)Nc1cc2c(cn1)C1(CC1)CN2c1cc(OCCN(C)C)nc(C(C)(F)F)n1] |
| | R—OH: 2-(dimethylamino)ethan-1-ol |
| | HPLC: Acidic conditions (Sunfire OBD 19), Gradient 5-35% |
| | 5.0 mg, LCMS m/z = 433.3 [M + H]⁺ |
| 141 | N-(1′-(2-(1,1-difluoroethyl)-6-(oxetan-3-yloxy)pyrimidin-4-yl)-1′,2′- |
| | dihydrospiro[cyclopropane-1,3′-pyrrolo[3,2-c]pyridin]-6′-yl)acetamide |
| | 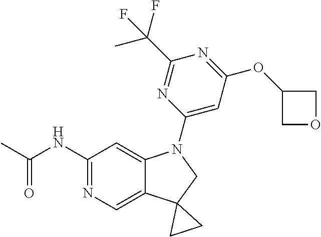 [SMILES: CC(=O)Nc1cc2c(cn1)C1(CC1)CN2c1cc(OC2COC2)nc(C(C)(F)F)n1] |
| | R—OH: oxetan-3-ol |
| | HPLC: Basic conditions (Sunfire OBD 19), Gradient 5-60% |
| | 5.9 mg, LCMS m/z = 418.2 [M + H]⁺ |
| 142 | N-(1′-(2-(1,1-difluoroethyl)-6-(pyrimidin-5-yloxy)pyrimidin-4-yl)-1′,2′- |
| | dihydrospiro[cyclopropane-1,3′-pyrrolo[3,2-c]pyridin]-6′-yl)acetamide |
| | trifluoroacetate |
| | 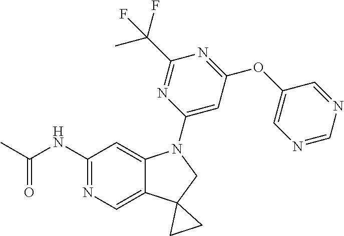 [SMILES: CC(=O)Nc1cc2c(cn1)C1(CC1)CN2c1cc(Oc2cncnc2)nc(C(C)(F)F)n1] |
| | R—OH: pyrimidin-5-ol |
| | HPLC: Acidic conditions (Sunfire OBD 19), Gradient 5-45% |
| | 7.2 mg, LCMS m/z = 440.2 [M + H]⁺ |
| 143 | N-(1′-(2-(1,1-difluoroethyl)-6-((1-methyl-1H-pyrazol-3-yl)oxy)pyrimidin-4- |
| | yl)-1′,2′-dihydrospiro[cyclopropane-1,3′-pyrrolo[3,2-c]pyridin]-6′- |
| | yl)acetamide trifluoroacetate |
| | 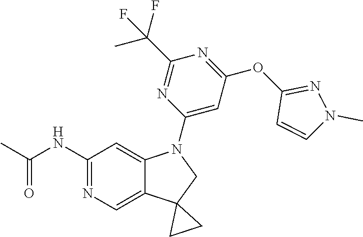 [SMILES: CC(=O)Nc1cc2c(cn1)C1(CC1)CN2c1cc(Oc2ccn(C)n2)nc(C(C)(F)F)n1] |
| | R—OH: 1-methyl-1H-pyrazol-3-ol |
| | HPLC: Acidic conditions (Sunfire OBD 19), Gradient 5-45% |
| | 8.8 mg, LCMS m/z = 442.2 [M + H]⁺ |
| 144 | N-(1′-(6-(1-cyanocyclopropoxy)-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | trifluoroacetate |
| | 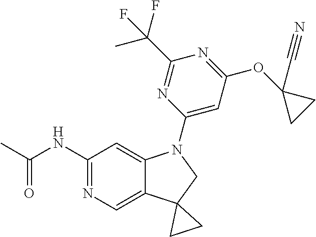 [SMILES: CC(=O)Nc1cc2c(cn1)C1(CC1)CN2c1cc(OC2(C#N)CC2)nc(C(C)(F)F)n1] |
| | R—OH: 1-hydroxycyclopropane-1-carbonitrile |
| | HPLC: Acidic conditions (Sunfire OBD 19), Gradient 5-60% |
| | 3.9 mg, LCMS m/z = 427.2 [M + H]⁺ |
| 145 | N-(1′-(2-(1,1-difluoroethyl)-6-((1-methyl-1H-pyrazol-4-yl)oxy)pyrimidin-4- |
| | yl)-1′,2′-dihydrospiro[cyclopropane-1,3′-pyrrolo[3,2-c]pyridin]-6′- |
| | yl)acetamide trifluoroacetate |
| | 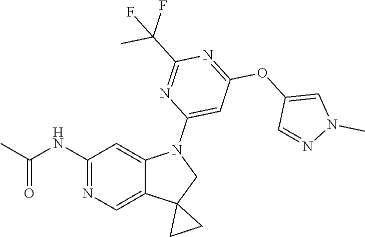 [SMILES: CC(=O)Nc1cc2c(cn1)C1(CC1)CN2c1cc(Oc2cnn(C)c2)nc(C(C)(F)F)n1] |
| | R—OH: 1-methyl-1H-pyrazol-4-ol |
| | HPLC: Acidic conditions (Sunfire OBD 19), Gradient 5-45% |
| | 9.8 mg, LCMS m/z = 442.2 [M + H]⁺ |
| 146 | N-(1′-(2-(1,1-difluoroethyl)-6-(pyrazin-2-yloxy)pyrimidin-4-yl)-1′,2′- |
| | dihydrospiro[cyclopropane-1,3′-pyrrolo[3,2-c]pyridin]-6′-yl)acetamide |
| | 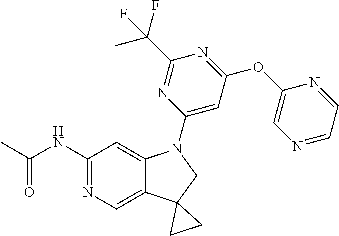 [SMILES: CC(=O)Nc1cc2c(cn1)C1(CC1)CN2c1cc(Oc2cnccn2)nc(C(C)(F)F)n1] |
| | R—OH: pyrazin-2-ol |
| | HPLC: Basic conditions (Sunfire OBD 19), Gradient 5-55% |
| | 3.9 mg, LCMS m/z = 440.2 [M + H]⁺ |
| 147 | N-(1′-(2-(1,1-difluoroethyl)-6-(pyridazin-4-yloxy)pyrimidin-4-yl)-1′,2′- |
| | dihydrospiro[cyclopropane-1,3′-pyrrolo[3,2-c]pyridin]-6′-yl)acetamide |
| | trifluoroacetate |
| | 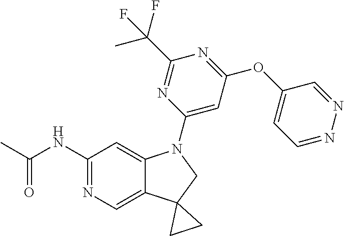 [SMILES: CC(=O)Nc1cc2c(cn1)C1(CC1)CN2c1cc(Oc2ccnnc2)nc(C(C)(F)F)n1] |
| | R—OH: pyridazin-4-ol |
| | HPLC: Acidic conditions (Sunfire OBD 19), Gradient 5-45% |
| | 0.9 mg, LCMS m/z = 440.2 [M + H]⁺ |
| 148 | N-(1′-(2-(1,1-difluoroethyl)-6-((1-ethylazetidin-3-yl)oxy)pyrimidin-4-yl)- |
| | 1′,2′-dihydrospiro[cyclopropane-1,3′-pyrrolo[3,2-c]pyridin]-6′-yl)acetamide |
| | trifluoroacetate |
| | 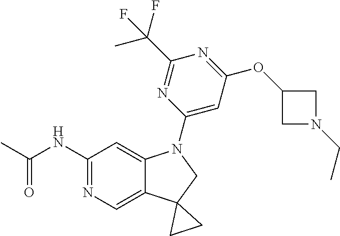 [SMILES: CCN1CC(Oc2cc(N3CC4(CC4)c4cnc(NC(C)=O)cc43)nc(C(C)(F)F)n2)C1] |
| | R—OH: 1-ethylazetidin-3-ol |
| | HPLC: Acidic conditions (Sunfire OBD 19), Gradient 5-55% |
| | 3.6 mg, LCMS m/z = 445.3 [M + H]⁺ |
| 149 | N-(1′-(2-(1,1-difluoroethyl)-6-((1-methyl-1H-1,2,3-triazol-5- |
| | yl)oxy)pyrimidin-4-yl)-1′,2′-dihydrospiro[cyclopropane-1,3′-pyrrolo[3,2- |
| | c]pyridin]-6′-yl)acetamide trifluorocetate |
| | 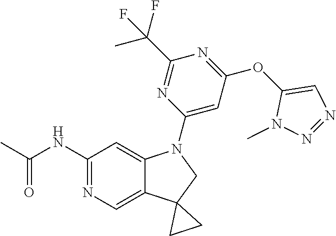 [SMILES: CC(=O)Nc1cc2c(cn1)C1(CC1)CN2c1cc(Oc2cnnn2C)nc(C(C)(F)F)n1] |
| | R—OH: 1-methyl-1H-1,2,3-triazol-5-ol |
| | HPLC: Acidic conditions (Sunfire OBD 19), Gradient 5-45% |
| | 3.1 mg, LCMS m/z = 443.2 [M + H]⁺ |
| 150 | Trans-rac-N-(1′-(2-(1,1-difluoroethyl)-6-(((3RS,4R)-4- |
| | fluorotetrahydrofuran-3-yl)oxy)pyrimidin-4-yl)-1′,2′- |
| | dihydrospiro[cyclopropane-1,3′-pyrrolo[3,2-c]pyridin]-6′-yl)acetamide |
| | trifluoroacetate |
| | 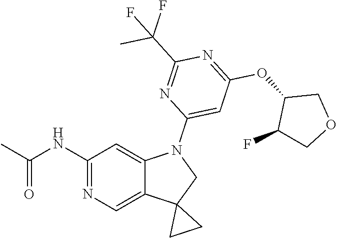 [SMILES: CC(=O)Nc1cc2c(cn1)C1(CC1)CN2c1cc(O[C@@H]2COC[C@H]2F)nc(C(C)(F)F)n1] |
| | R—OH: trans-rac-(3RS,4R)-4-fluorotetrahydrofuran-3-ol |
| | HPLC: Acidic conditions (Sunfire OBD 19), Gradient 5-50% |
| | 1.3 mg, LCMS m/z = 415.2 [M + H]⁺ |
| 151 | N-(1′-(6-(1-cyanoethoxy)-2-(1,1-difluoroethyl)pyrimidin-4-yl)-1′,2′- |
| | dihydrospiro[cyclopropane-1,3′-pyrrolo[3,2-c]pyridin]-6′-yl)acetamide |
| | trifluoroacetate |
| | 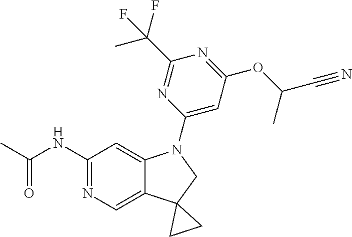 [SMILES: CC(=O)Nc1cc2c(cn1)C1(CC1)CN2c1cc(OC(C)C#N)nc(C(C)(F)F)n1] |
| | R—OH: 2-hydroxypropanenitrile |
| | HPLC: Acidic conditions (Sunfire OBD 19), Gradient 5-65% |
| | 3.1 mg, LCMS m/z = 415.2 [M + H]⁺ |
| 152 | N-(1′-(6-(cyano(cyclopropyl)methoxy)-2-(1,1-difluoroethyl)pyrimidin-4-yl)- |
| | 1′,2′-dihydrospiro[cyclopropane-1,3′-pyrrolo[3,2-c]pyridin]-6′-yl)acetamide |
| | trifluoroacetate |
| | 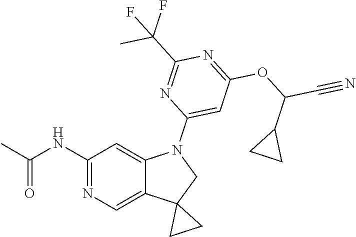 [SMILES: CC(=O)Nc1cc2c(cn1)C1(CC1)CN2c1cc(OC(C#N)C2CC2)nc(C(C)(F)F)n1] |
| | R—OH: 2-cyclopropyl-2-hydroxyacetonitrile |
| | HPLC: Acidic conditions (Sunfire OBD 19), Gradient 5-70% |
| | 1.9 mg, LCMS m/z = 441.2 [M + H]⁺ |

### Examples 153 to 182

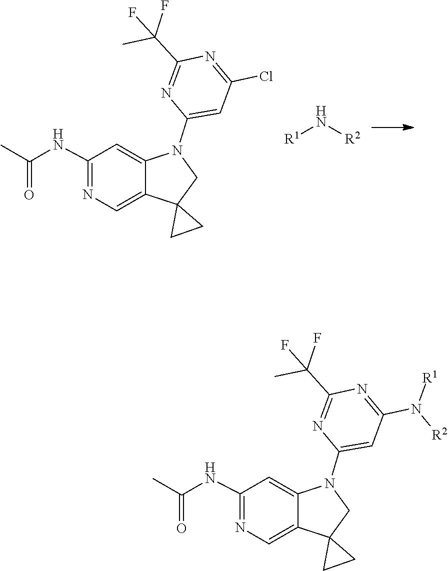

In a 2-dram vial, a solution of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 19.0 mg, 50 μmol), R¹R²NH (75 μmol) and K₂CO₃ (20.7 mg, 150 μmol) in DMF (1 mL, 0.2 M) was sealed with a Teflon cap, and the reaction solution was heated at 50° C. for 16 h. After cooling to rt, the reaction mixture was diluted EtOAc, washed with H₂O (3 mL), and extracted with EtOAc (5 mL, 2×). The combined organic phases were dried over anhydrous MgSO₄ and concentrated in vacuo to provide the crude product. This was purified by HPLC using a Waters SunFire Prep C18 column (5 um OBD 19×100 mm) underbasic or acidic conditions, or Waters XSelect CSH Prep C18 column (5 um OBD 19×100 mm
Basic conditions (Sunfire OBD 19): MeCM, water, 0.2% NH₄OH modifier
Basic Conditions (Xselect OBD 19): MeCN, water, 0.2% NH₄OH modifier
Acidic conditions (Sunfire OBD 19): MeCN, water, 0.1% TFA modifier

| Example No | Name, Structure, R¹R²NH, HPLC, Data |
|---|---|
| 153 | N-(1′-(2-(1,1-difluoroethyl)-6-(isopropylamino) |
| | pyrimidin-4-yl)-1′,2′-dihydrospiro[cyclopropane- |
| | 1,3′-pyrrolo[3,2-c]pyridin]-6′-yl)acetamide trifluoroacetate |
| | 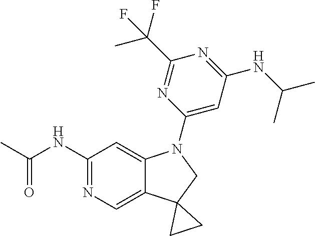 [SMILES: CC(=O)Nc1cc2c(cn1)C1(CC1)CN2c1cc(NC(C)C)nc(C(C)(F)F)n1] |
| | R¹R²NH: propan-2-amine |
| | HPLC: Acidic conditions (Sunfire OBD 19), Gradient 5-65% |
| | 3.1 mg, LCMS m/z = 403.3 [M + H]⁺ |
| 154 | N-(1′-(6-((cyclopropylmethyl)amino)-2-(1,1-difluoroethyl) |
| | pyrimidin-4-yl)-1′,2′-dihydrospiro[cyclopropane-1,3′- |
| | pyrrolo[3,2-c]pyridin]-6′-yl)acetamide trifluoroacetate |
| | 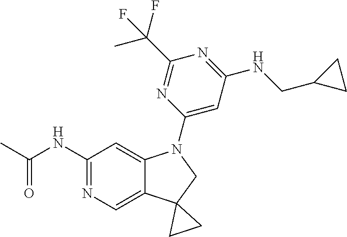 [SMILES: CC(=O)Nc1cc2c(cn1)C1(CC1)CN2c1cc(NCC2CC2)nc(C(C)(F)F)n1] |
| | R¹R²NH: cyclopropylmethylamine |
| | HPLC: Acidic conditions (Sunfire OBD 19), Gradient 5-65% |
| | 4.6 mg, LCMS m/z = 415.3 [M + H]⁺ |
| 155 | N-(1′-(2-(1,1-difluoroethyl)-6-((tetrahydro-2H-pyran- |
| | 4-yl)amino)pyrimidin-4-yl)-1′,2′- |
| | dihydrospiro[cyclopropane-1,3′-pyrrolo[3,2-c]pyridin]-6′- |
| | yl)acetamide |
| | 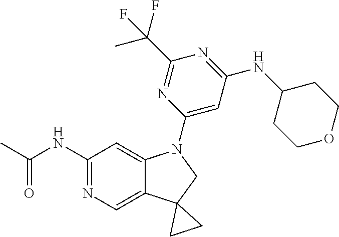 [SMILES: CC(=O)Nc1cc2c(cn1)C1(CC1)CN2c1cc(NC2CCOCC2)nc(C(C)(F)F)n1] |
| | R¹R²NH: tetrahydro-2H-pyran-4-amine |
| | HPLC: Basic conditions (Xselect OBD 19), Gradient 5-60% |
| | 2.1 mg, LCMS m/z = 445.3 [M + H]⁺ |
| 156 | N-(1′-(2-(1,1-difluoroethyl)-6-(propylamino)pyrimidin-4-yl)- |
| | 1′,2′-dihydrospiro[cyclopropane-1,3′-pyrrolo[3,2-c]pyridin]- |
| | 6′-yl)acetamide trifluoroacetate |
| | 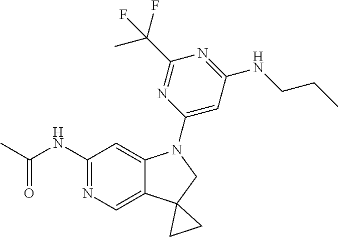 [SMILES: CCCNc1cc(N2CC3(CC3)c3cnc(NC(C)=O)cc32)nc(C(C)(F)F)n1] |
| | R¹R²NH: propylamine |
| | HPLC: Acidic conditions (Sunfire OBD 19), Gradient 5-65% |
| | 7.5 mg, LCMS m/z = 403.2 [M + H]⁺ |
| 157 | N-(1′-(6-(cyclobutylamino)-2-(1,1-difluoroethyl)pyrimidin- |
| | 4-yl)-1′,2′-dihydrospiro[cyclopropane- |
| | 1,3′-pyrrolo[3,2-c]pyridin]-6′-yl)acetamide |
| | trifluoroacetate |
| | 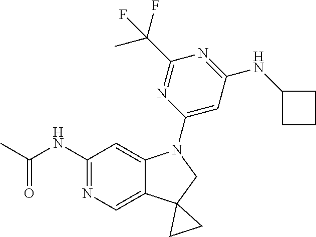 [SMILES: CC(=O)Nc1cc2c(cn1)C1(CC1)CN2c1cc(NC2CCC2)nc(C(C)(F)F)n1] |
| | R¹R²NH: cyclobutylamine |
| | HPLC: Acidic conditions (Sunfire OBD 19), Gradient 5-65% |
| | 4.2 mg, LCMS m/z = 415.3 [M + H]⁺ |
| 158 | N-(1′-(2-(1,1-difluoroethyl)-6-((2-methoxy-2- |
| | methylpropyl)amino)pyrimidin-4-yl)-1′, |
| | 2′-dihydrospiro[cyclopropane-1,3′- |
| | pyrrolo[3,2-c]pyridin]-6′-yl)acetamide trifluoroacetate |
| | 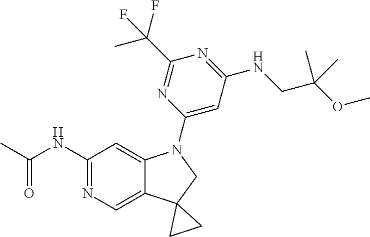 [SMILES: COC(C)(C)CNc1cc(N2CC3(CC3)c3cnc(NC(C)=O)cc32)nc(C(C)(F)F)n1] |
| | R¹R²NH: 2-methoxy-2-methylpropan-1-amine |
| | HPLC: Acidic conditions (Sunfire OBD 19), Gradient 5-65% |
| | 2.3 mg, LCMS m/z = 447.3 [M + H]⁺ |
| 159 | N-(1′-(6-(bicyclo[1.1.1]pentan-1-ylamino)-2-(1,1- |
| | difluoroethyl)pyrimidin-4-yl)-1′,2′- |
| | dihydrospiro[cyclopropane-1,3′-pyrrolo[3,2-c]pyridin]-6′- |
| | yl)acetamide trifluoroacetate |
| | 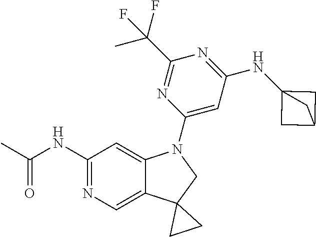 [SMILES: CC(=O)Nc1cc2c(cn1)C1(CC1)CN2c1cc(NC23CC(C2)C3)nc(C(C)(F)F)n1] |
| | R¹R²NH: bicyclo[1.1.1]pentan-1-amine |
| | HPLC: Acidic conditions (Sunfire OBD 19), Gradient 5-65% |
| | 0.2 mg, LCMS m/z = 427.2 [M + H]⁺; |
| 160 | N-(1′-(2-(1,1-difluoroethyl)-6-(((1- |
| | fluorocyclopropyl)methyl)amino)pyrimidin-4-yl)-1′,2′- |
| | dihydrospiro[cyclopropane-1,3′-pyrrolo[3,2-c]pyridin]-6′- |
| | yl)acetamide trifluoroacetate |
| | 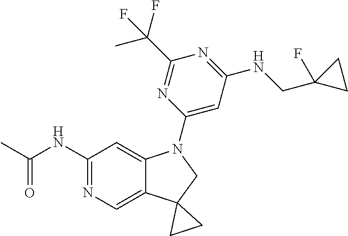 [SMILES: CC(=O)Nc1cc2c(cn1)C1(CC1)CN2c1cc(NCC2(F)CC2)nc(C(C)(F)F)n1] |
| | R¹R²NH: (1-fluorocyclopropyl)methylamine |
| | HPLC: Acidic conditions (Sunfire OBD 19), Gradient 5-55% |
| | 2.5 mg, LCMS m/z = 433.2 [M + H]⁺ |
| 161 | N-(1′-(2-(1,1-difluoroethyl)-6-(((1- |
| | methoxycyclopropyl)methyl)amino)pyrimidin-4-yl)-1′,2′- |
| | dihydrospiro[cyclopropane-1,3′-pyrrolo[3,2-c] |
| | pyridin]-6′-yl)acetamide trifluoroacetate |
| | 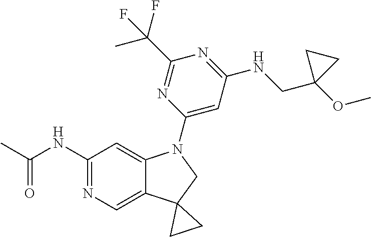 [SMILES: COC1(CNc2cc(N3CC4(CC4)c4cnc(NC(C)=O)cc43)nc(C(C)(F)F)n2)CC1] |
| | R¹R²NH: (1-methoxycyclopropyl)methanamine |
| | HPLC: Acidic conditions (Sunfire OBD 19), Gradient 5-60% |
| | 5.1 mg, LCMS m/z = 445.2 [M + H]⁺ |
| 162 | (R)-N-(1′-(2-(1,1-difluoroethyl)-6-((oxetan-2-ylmethyl) |
| | amino)pyrimidin-4-yl)-1′,2′-dihydrospiro |
| | [cyclopropane-1,3′-pyrrolo[3,2-c]pyridin]-6′- |
| | yl)acetamide |
| | 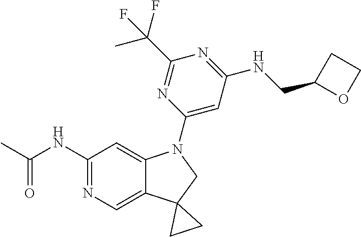 [SMILES: CC(=O)Nc1cc2c(cn1)C1(CC1)CN2c1cc(NC[C@H]2CCO2)nc(C(C)(F)F)n1] |
| | R¹R²NH: (R)-oxetan-2-ylmethanamine |
| | HPLC: Basic conditions (Xselect OBD 19), Gradient 5-55% |
| | 3.5 mg, LCMS m/z = 431.3 [M + H]⁺ |
| 163 | (S)-N-(1′-(2-(1,1-difluoroethyl)-6-((oxetan-2-ylmethyl) |
| | amino)pyrimidin-4-yl)-1′,2′-dihydrospiro |
| | [cyclopropane-1,3′-pyrrolo[3,2-c]pyridin]-6′- |
| | yl)acetamide |
| | 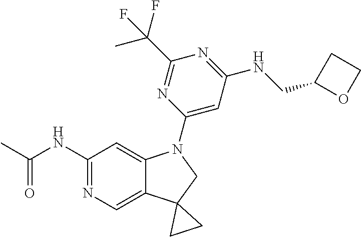 [SMILES: CC(=O)Nc1cc2c(cn1)C1(CC1)CN2c1cc(NC[C@@H]2CCO2)nc(C(C)(F)F)n1] |
| | R¹R²NH: (S)-oxetan-2-ylmethanamine |
| | HPLC: Basic conditions (Xselect OBD 19), Gradient 5-55% |
| | 3.5 mg, LCMS m/z = 431.3 [M + H]⁺ |
| 164 | N-(1′-(2-(1,1-difluoroethyl)-6-((3-fluoropropyl)amino) |
| | pyrimidin-4-yl)-1′,2′-dihydrospiro |
| | [cyclopropane-1,3′-pyrrolo[3,2-c]pyridin]-6′-yl)acetamide |
| | trifluoroacetate |
| | 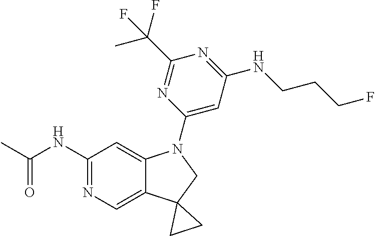 [SMILES: CC(=O)Nc1cc2c(cn1)C1(CC1)CN2c1cc(NCCCF)nc(C(C)(F)F)n1] |
| | R¹R²NH: 3-fluoropropan-1-amine |
| | HPLC: Acidic conditions (Sunfire OBD 19), Gradient 5-60% |
| | 5.5 mg, ICMS m/z = 421.3 [M + H]⁺ |
| 165 | N-(1′-(2-(1,1-difluoroethyl)-6-(((2S,3S)-3-methoxybutan-2- |
| | yl)amino)pyrimidin-4-yl)-1′,2′-dihydrospiro |
| | [cyclopropane-1,3′-pyrrolo[3,2- |
| | c]pyridin]-6′-yl)acetamide trifluoroacetate |
| | 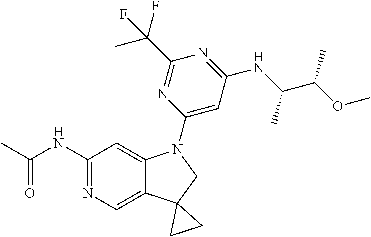 [SMILES: CO[C@@H](C)[C@H](C)Nc1cc(N2CC3(CC3)c3cnc(NC(C)=O)cc32)nc(C(C)(F)F)n1] |
| | R¹R²NH: (2S,3S)-3-methoxybutan-2-amine |
| | HPLC: Acidic conditions (Sunfire OBD 19), Gradient 5-65% |
| | 1.5 mg, LCMS m/z = 447.3 [M + H]⁺ |
| 166 | N-(1′-(2-(1,1-difluoroethyl)-6-(thietan-3-ylamino)pyrimidin- |
| | 4-yl)-1′,2′-dihydrospiro[cyclopropane- |
| | 1,3′-pyrrolo[3,2-c]pyridin]-6′-yl)acetamide |
| | trifluoroacetate |
| | 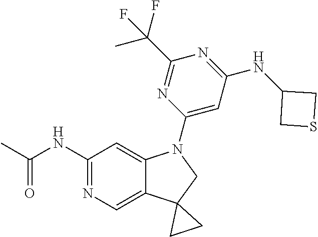 [SMILES: CC(=O)Nc1cc2c(cn1)C1(CC1)CN2c1cc(NC2CSC2)nc(C(C)(F)F)n1] |
| | R¹R²NH: thietan-3-amine |
| | HPLC: Acidic conditions (Sunfire OBD 19), Gradient 5-60% |
| | 1.0 mg, LCMS m/z = 433.2 [M + H]⁺ |
| 167 | N-(1′-(2-(1,1-difluoroethyl)-6-(ethylamino) |
| | pyrimidin-4-yl)-1′,2′-dihydrospiro |
| | [cyclopropane-1,3′-pyrrolo[3,2-c]pyridin]-6′-yl) |
| | acetamide trifluoroacetate |
| | 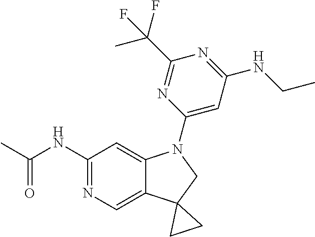 [SMILES: CCNc1cc(N2CC3(CC3)c3cnc(NC(C)=O)cc32)nc(C(C)(F)F)n1] |
| | R¹R²NH: ethylamine |
| | HPLC: Acidic conditions (Sunfire OBD 19), Gradient 5-60% |
| | 0.6 mg, LCMS m/z = 389.2 [M + H]⁺ |
| 168 | N-(1′-(2-(1,1-difluoroethyl)-6-(((1s,3s)-3- |
| | fluorocyclobutyl)amino)pyrimidin-4-yl)-1′,2′- |
| | dihydrospiro[cyclopropane-1,3′-pyrrolo |
| | [3,2-c]pyridin]-6′-yl)acetamide trifluoroacetate |
| | 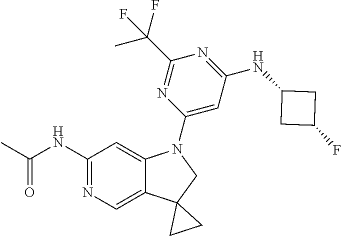 [SMILES: CC(=O)Nc1cc2c(cn1)C1(CC1)CN2c1cc(N[C@H]2C[C@@H](F)C2)nc(C(C)(F)F)n1] |
| | R¹R²NH: (1s,3s)-3-fluorocyclobutan-1-amine |
| | HPLC: Acidic conditions (Sunfire OBD 19), Gradient 5-65% |
| | 2.2 mg, LCMS m/z = 433.2 [M + H]⁺ |
| 169 | N-(1′-(2-(1,1-difluoroethyl)-6-((2-fluoropropyl)amino) |
| | pyrimidin-4-yl)-1′,2′-dihydrospiro[cyclopropane- |
| | 1,3′-pyrrolo[3,2-c]pyridin]-6′-yl)acetamide |
| | trifluoroacetate |
| | 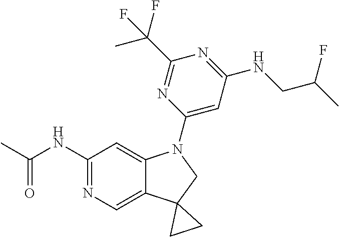 [SMILES: CC(=O)Nc1cc2c(cn1)C1(CC1)CN2c1cc(NCC(C)F)nc(C(C)(F)F)n1] |
| | R¹R²NH: 2-fluoropropan-1-amine |
| | HPLC: Acidic conditions (Sunfire OBD 19), Gradient 5-60% |
| | 2.8 mg, LCMS m/z = 421.3 [M + H]⁺ |
| 170 | N-(1′-(2-(1,1-difluoroethyl)-6-((3-fluorobutyl)amino) |
| | pyrimidin-4-yl)-1′,2′-dihydrospiro[cyclopropane-1,3′- |
| | pyrrolo[3,2-c]pyridin]-6′-yl)acetamide trifluoroacetate |
| | 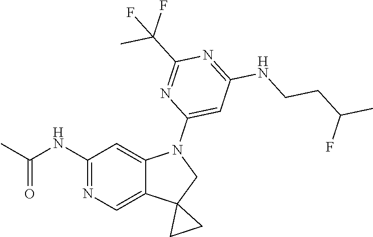 [SMILES: CC(=O)Nc1cc2c(cn1)C1(CC1)CN2c1cc(NCCC(C)F)nc(C(C)(F)F)n1] |
| | R¹R²NH: 3-fluorobutan-1-amine |
| | HPLC: Acidic conditions (Sunfire OBD 19), Gradient 5-65% |
| | 5.7 mg, LCMS m/z = 435.3 [M + H]⁺ |
| 171 | N-(1′-(2-(1,1-difluoroethyl)-6-((2-methoxypropyl)amino) |
| | pyrimidin-4-yl)-1′,2′-dihydrospiro[cyclopropane-1,3′- |
| | pyrrolo[3,2-c]pyridin]-6′-yl)acetamide trifluoroacetate |
| | 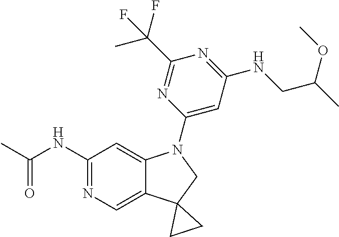 [SMILES: COC(C)CNc1cc(N2CC3(CC3)c3cnc(NC(C)=O)cc32)nc(C(C)(F)F)n1] |
| | R¹R²NH: 2-methoxypropan-1-amine |
| | HPLC: Acidic conditions (Sunfire OBD 19), Gradient 5-60% |
| | 5.5 mg, LCMS m/z = 433.3 [M + H]⁺ |
| 172 | N-(1′-(2-(1,1-difluoroethyl)-6-(((1s,3s)-3- |
| | methoxycyclobutyl)amino)pyrimidin-4-yl)-1′,2′-dihydrospiro |
| | [cyclopropane-1,3′-pyrrolo[3,2-c]pyridin]-6′-yl) |
| | acetamide trifluoroacetate |
| | 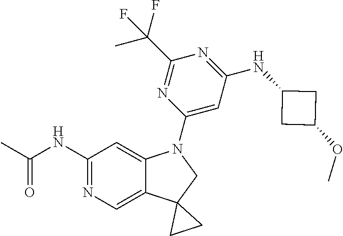 [SMILES: CO[C@H]1C[C@@H](Nc2cc(N3CC4(CC4)c4cnc(NC(C)=O)cc43)nc(C(C)(F)F)n2)C1] |
| | R¹R²NH: (1s,3s)-3-methoxycyclobutan-1-amine |
| | HPLC: Acidic conditions (Sunfire OBD 19), Gradient 5-60% |
| | 5.6 mg, LCMS m/z = 445.3 [M + H]⁺ |
| 173 | N-(1′-(2-(1,1-difluoroethyl)-6-((2-methoxy-2- |
| | methylcyclopropyl)amino)pyrimidin-4-yl)-1′,2′-dihydrospiro |
| | [cyclopropane-1,3′-pyrrolo[3,2-c]pyridin]-6′-yl)acetamide |
| | trifluoroacetate |
| | 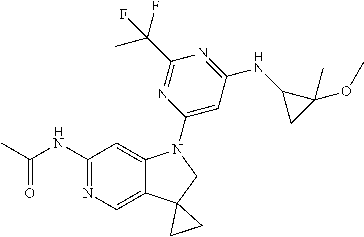 [SMILES: COC1(C)CC1Nc1cc(N2CC3(CC3)c3cnc(NC(C)=O)cc32)nc(C(C)(F)F)n1] |
| | R¹R²NH: 2-methoxy-2-methylcyclopropan-1-amine |
| | HPLC: Acidic conditions (Sunfire OBD 19), Gradient 5-60% |
| | 3.4 mg, LCMS m/z = 445.3 [M + H]⁺ |
| 174 | N-(1′-(2-(1,1-difluoroethyl)-6-(((1-methylazetidin-2- |
| | yl)methyl)amino)pyrimidin-4-yl)-1′,2′-dihydrospiro |
| | [cyclopropane-1,3′-pyrrolo[3,2-c]pyridin]-6′-yl)acetamide |
| | 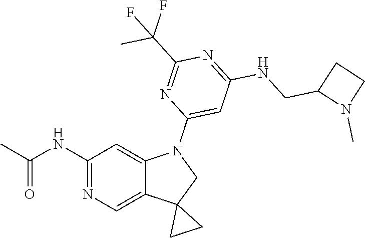 [SMILES: CC(=O)Nc1cc2c(cn1)C1(CC1)CN2c1cc(NCC2CCN2C)nc(C(C)(F)F)n1] |
| | R¹R²NH: (1-methylazetidin-2-yl)methanamine |
| | HPLC: Basic conditions (Xselect OBD 19), Gradient 5-55% |
| | 2.5 mg, LCMS m/z = 444.3 [M + H]⁺ |
| 175 | N-(1′-(2-(1,1-difluoroethyl)-6-((1-(oxetan-3-yl)ethyl) |
| | amino)pyrimidin-4-yl)-1′,2′-dihydrospiro |
| | [cyclopropane-1,3′-pyrrolo[3,2-c]pyridin]-6′-yl)acetamide |
| | 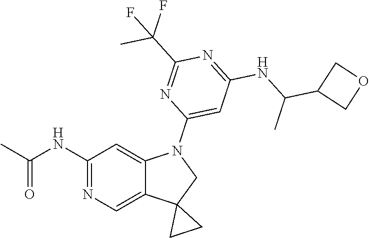 [SMILES: CC(=O)Nc1cc2c(cn1)C1(CC1)CN2c1cc(NC(C)C2COC2)nc(C(C)(F)F)n1] |
| | R¹R²NH: 1-(oxetan-3-yl)ethan-1-amine |
| | HPLC: Basic conditions (Xselect OBD 19), Gradient 5-55% |
| | 1.2 mg, LCMS m/z = 445.2 [M + H]⁺ |
| 176 | N-(1′-(2-(1,1-difluoroethyl)-6-(spiro[2.2]pentan-1- |
| | ylamino)pyrimidin-4-yl)-1′,2′-dihydrospiro |
| | [cyclopropane-1,3′-pyrrolo[3,2-c]pyridin]-6′-yl)acetamide |
| | trifluoroacetate |
| | 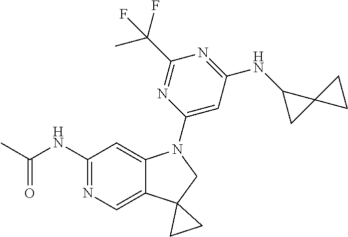 [SMILES: CC(=O)Nc1cc2c(cn1)C1(CC1)CN2c1cc(NC2CC23CC3)nc(C(C)(F)F)n1] |
| | R¹R²NH: spiro[2.2]pentan-1-amine |
| | HPLC: Acidic conditions (Sunfire OBD 19), Gradient 5-70% |
| | 3.8 mg, LCMS m/z = 427.2 [M + H]⁺ |
| 177 | N-(1′-(2-(1,1-difluoroethyl)-6-((2-methoxycyclopropyl) |
| | amino)pyrimidin-4-yl)-1′,2′-dihydrospiro |
| | [cyclopropane-1,3′-pyrrolo[3,2-c]pyridin]-6′- |
| | yl)acetamide |
| | 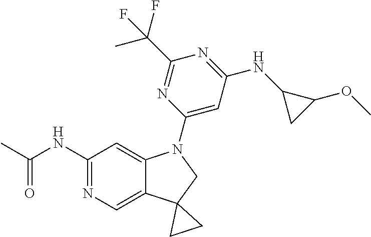 [SMILES: COC1CC1Nc1cc(N2CC3(CC3)c3cnc(NC(C)=O)cc32)nc(C(C)(F)F)n1] |
| | R¹R²NH: 2-methoxycyclopropan-1-amine |
| | HPLC: Basic conditions (Xselect OBD 19), Gradient 5-60% |
| | 20 mg, LCMS m/z = 431.2 [M + H]⁺ |
| 178 | N-(1′-(2-(1,1-difluoroethyl)-6-((1-methoxypropan- |
| | 2-yl)amino)pyrimidin-4-yl)-1′,2′-dihydrospiro |
| | [cyclopropane-1,3′-pyrrolo[3,2-c]pyridin]-6′- |
| | yl)acetamide trifluoroacetate |
| | 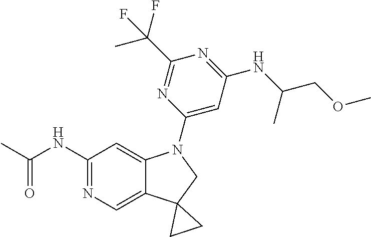 [SMILES: COCC(C)Nc1cc(N2CC3(CC3)c3cnc(NC(C)=O)cc32)nc(C(C)(F)F)n1] |
| | R¹R²NH: 1-methoxypropan-2-amine |
| | HPLC: Acidic conditions (Sunfire OBD 19), Gradient 5-60% |
| | 1.6 mg, LCMS m/z = 433.3 [M + H]⁺ |
| 179 | N-(1′-(2-(1,1-difluoroethyl)-6-((tetrahydrofuran-3-yl) |
| | amino)pyrimidin-4-yl)-1′,2′-dihydrospiro[cyclopropane- |
| | 1,3′-pyrrolo[3,2-c]pyridin]-6′-yl)acetamide |
| | trifluoroacetate |
| | 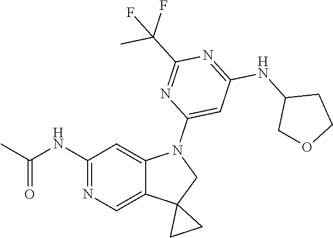 [SMILES: CC(=O)Nc1cc2c(cn1)C1(CC1)CN2c1cc(NC2CCOC2)nc(C(C)(F)F)n1] |
| | R¹R²NH: tetrahydrofuran-3-amine |
| | HPLC: Acidic conditions (Sunfire OBD 19), Gradient 5-55% |
| | 3.1 mg, LCMS m/z = 431.3 [M + H]⁺ |
| 180 | N-(1′-(2-(1,1-difluoroethyl)-6-((2- |
| | (dimethylamino)propyl)amino)pyrimidin- |
| | 4-yl)-1′,2′-dihydrospiro[cyclopropane-1,3′-pyrrolo |
| | [3,2-c]pyridin]-6′-yl)acetamide trifluoroacetate |
| | 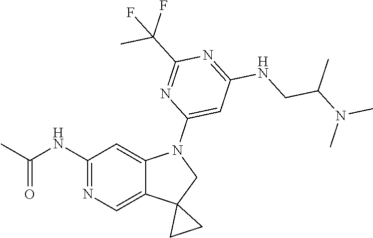 [SMILES: CC(=O)Nc1cc2c(cn1)C1(CC1)CN2c1cc(NCC(C)N(C)C)nc(C(C)(F)F)n1] |
| | R¹R²NH: N2,N2-dimethylpropane-1,2-diamine |
| | HPLC: Acidic conditions (Sunfire OBD 19), Gradient 5-60% |
| | 6.5 mg, LCMS m/z = 446.3 [M + H]⁺ |
| 181 | N-(1′-(2-(1,1-difluoroethyl)-6-((tetrahydrothiophen- |
| | 3-yl)amino)pyrimidin-4- |
| | yl)-1′,2′-dihydrospiro[cyclopropane-1,3′-pyrrolo |
| | [3,2-c]pyridin]-6′-yl)acetamide |
| | 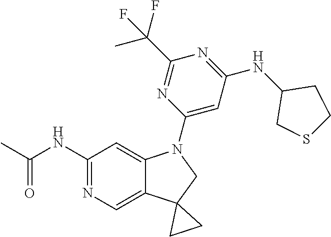 [SMILES: CC(=O)Nc1cc2c(cn1)C1(CC1)CN2c1cc(NC2CCSC2)nc(C(C)(F)F)n1] |
| | HPLC: Basic conditions (Xselect OBD 19), Gradient 5-65% |
| | 1.5 mg, LCMS m/z = 447.2 [M + H]⁺ |
| 182 | N-(1′-(2-(1,1-difluoroethyl)-6-((2- |
| | methylcyclopropyl)amino)pyrimidin-4- |
| | yl)-1′,2′-dihydrospiro[cyclopropane-1,3′-pyrrolo |
| | [3,2-c]pyridin]-6′-yl)acetamide trifluoroacetate |
| | 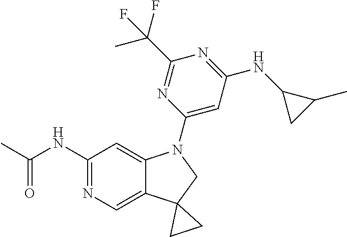 [SMILES: CC(=O)Nc1cc2c(cn1)C1(CC1)CN2c1cc(NC2CC2C)nc(C(C)(F)F)n1] |
| | R¹R²NH: 2-methylcyclopropan-1-amine |
| | HPLC: Acidic conditions (Sunfire OBD 19), Gradient 5-65% |
| | 3.9 mg, LCMS m/z = 415.3 [M + H]⁺ |

### Examples 182 to 208

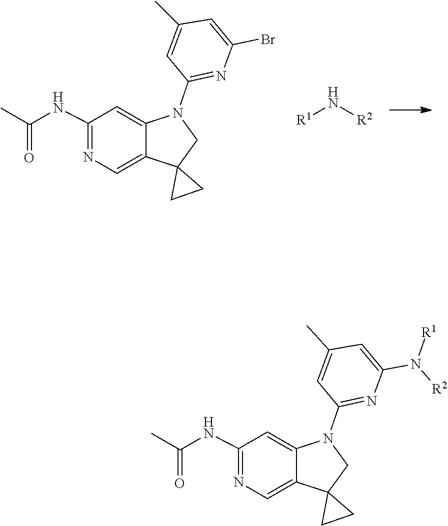

An 8 mL vial was charged with N-(1′-(6-bromo-4-methylpyridin-2-yl)-1′,2′-dihydrospiro[cyclopropane-1,3′-pyrrolo[3,2-c]pyridin]-6′-yl)acetamide (Preparation 157, 28.0 mg, 75 μmol), R¹R²NH (75 μmol), Xantphos Pd G3 (7.1 mg, 7.5 μmol), Xantphos (4.3 mg, 7.5 μmol) and NaOtBu (21.6 mg, 225 μmol). Dioxane (1.5 mL, 0.2 M) was added under an inert atmosphere, the vial was sealed with a Teflon cap, and the reaction solution was heated under reflux at 90° C. for 16 h. The reaction was cooled to rt, the reaction mixture was diluted EtOAc, washed with H₂O (3 mL), and extracted with EtOAc (5 mL, 2×). The combined organic phases were dried over anhydrous MgSO₄ and concentrated in vacuo to give the crude product. This was purified by HPLC using a Waters SunFire Prep C18 column (5 um OBD 19×100 mm) under acidic conditions, or a Waters XSelect CSH Prep C18 column (5 um OBD 19×100 mm) or (5 um OBD 30×100 mm) under basic conditions.
Basic conditions (Xselect OBD 19): MeCN, water, 0.2% NH₄OH modifier
Basic conditions (Xselect OBD 30): MeCN, water, 0.2% NH₄OH modifier
Acidic conditions (Sunfire OBD 19): MeCN, water, 0.1% TFA modifier

| Example | |
|---|---|
| No | Name, Structure, R¹R²NH, HPLC, Data |
| 183 | N-(1′-(6-(3-(difluoromethyl)-4-methylpiperazin-1-yl)- |
| | 4-methylpyridin-2-yl)-1′,2′-dihydrospiro[cyclopropane- |
| | 1,3′-pyrrolo[3,2-c]pyridin]-6′-yl)acetamide |
| | 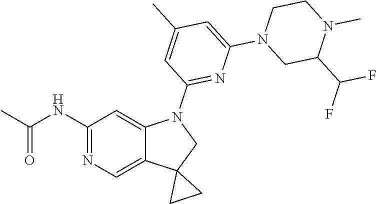 [SMILES: CC(=O)Nc1cc2c(cn1)C1(CC1)CN2c1cc(C)cc(N2CCN(C)C(C(F)F)C2)n1] |
| | R¹R²NH: 2-(difluoromethyl)-1-methylpiperazine |
| | HPLC: Basic conditions (Xselect OBD 19): Gradient 5-60% |
| | 3.3 mg, LCMS m/z = 443.3 [M + H]⁺ |
| 184 | N-(1′-(6-(3,3-difluoropyrrolidin-1-yl)-4-methylpyridin- |
| | 2-yl)-1′,2′- |
| | dihydrospiro[cyclopropane-1,3′-pyrrolo[3,2-c]pyridin]- |
| | 6′-yl)acetamide |
| | 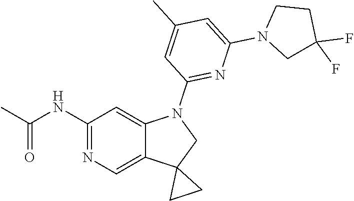 [SMILES: CC(=O)Nc1cc2c(cn1)C1(CC1)CN2c1cc(C)cc(N2CCC(F)(F)C2)n1] |
| | R¹R²NH: 3,3-difluoropyrrolidine |
| | HPLC: Basic conditions (Xselect OBD 30): gradient: 10-70% |
| | 7.40 mg, LCMS m/z = 400.3 [M + H]⁺ |
| 185 | N-(1′-(6-(3-(2-methoxyethoxy)pyrrolidin-1-yl)-4- |
| | methylpyridin-2-yl)-1′,2′-dihydrospiro |
| | [cyclopropane-1,3′-pyrrolo[3,2-c]pyridin]-6′-yl)acetamide |
| | 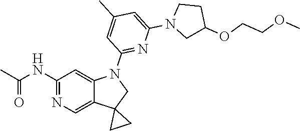 [SMILES: COCCOC1CCN(c2cc(C)cc(N3CC4(CC4)c4cnc(NC(C)=O)cc43)n2)C1] |
| | R¹R²NH: 3-(2-methoxyethoxy)pyrrolidine |
| | HPLC: Basic conditions (Xselect OBD 30): gradient: 10-65% |
| | 6.4 mg, LCMS m/z = 438.3 [M + H]⁺ |
| 186 | N-(1′-(6-(3-hydroxy-3-(trifluoromethyl)pyrrolidin- |
| | 1-yl)-4-methylpyridin-2-yl)-1′,2′- |
| | dihydrospiro[cyclopropane-1,3′-pyrrolo[3,2-c]pyridin]-6′- |
| | yl)acetamide |
| | 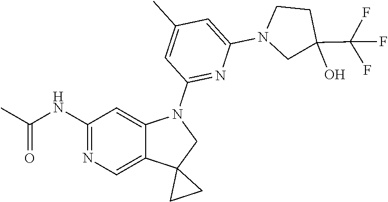 [SMILES: CC(=O)Nc1cc2c(cn1)C1(CC1)CN2c1cc(C)cc(N2CCC(O)(C(F)(F)F)C2)n1] |
| | R¹R²NH: 3-hydroxy-3-(trifluoromethyl)pyrrolidine |
| | HPLC: Basic conditions (Xselect OBD 30): gradient: 5-70% |
| | 10.2 mg, LCMS m/z = 448.2 [M + H]⁺ |
| 187 | N-(1′-(6-(3,4-dihydroxypyrrolidin-1-yl)-4-methylpyridin- |
| | 2-yl)-1′,2′-dihydrospiro[cyclopropane- |
| | 1,3′-pyrrolo[3,2-c]pyridin]-6′-yl)acetamide |
| | 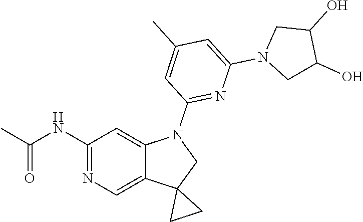 [SMILES: CC(=O)Nc1cc2c(cn1)C1(CC1)CN2c1cc(C)cc(N2CC(O)C(O)C2)n1] |
| | R¹R²NH: 3,4-dihydroxypyrrolidine |
| | HPLC: Basic conditions (Xselect OBD 30): gradient: 5-50% |
| | 5.7 mg, LCMS m/z = 396.3 [M + H]⁺ |
| 188 | N-(1′-(6-(3-methoxypiperidin-1-yl)-4-methylpyridin-2-yl)- |
| | 1′,2′-dihydrospiro[cyclopropane-1,3′- |
| | pyrrolo[3,2-c]pyridin]-6′-yl)acetamide |
| | 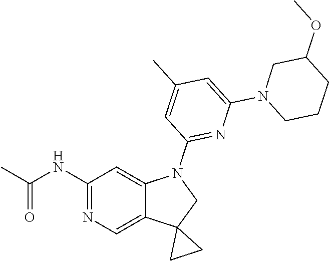 [SMILES: COC1CCCN(c2cc(C)cc(N3CC4(CC4)c4cnc(NC(C)=O)cc43)n2)C1] |
| | R¹R²NH: 3-methoxypiperidine |
| | HPLC: Basic conditions (Xselect OBD 30): gradient: 5-70% |
| | 3.0 mg, LCMS m/z = 408.3 [M + H]⁺ |
| 189 | N-(1′-(6-(3-hydroxypyrrolidin-1-yl)-4-methylpyridin- |
| | 2-yl)-1′,2′-dihydrospiro[cyclopropane- |
| | 1,3′-pyrrolo[3,2-c]pyridin]-6′-yl)acetamide |
| | 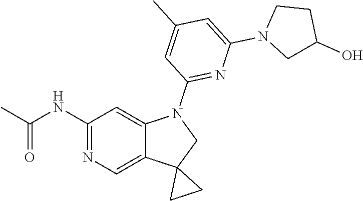 [SMILES: CC(=O)Nc1cc2c(cn1)C1(CC1)CN2c1cc(C)cc(N2CCC(O)C2)n1] |
| | R¹R²NH: 3-hydroxypyrrolidine |
| | HPLC: Basic conditions (Xselect OBD 19): Gradient 5-60% |
| | 9.7 mg, LCMS m/z = 380.2 [M + H]⁺ |
| 190 | N-(1′-(6-(dimethylamino)-4-methylpyridin-2-yl)-1′,2′- |
| | dihydrospiro[cyclopropane-1,3′-pyrrolo[3,2-c]pyridin]-6′- |
| | yl)acetamide |
| | 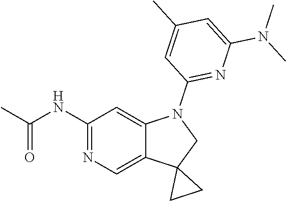 [SMILES: CC(=O)Nc1cc2c(cn1)C1(CC1)CN2c1cc(C)cc(N(C)C)n1] |
| | R¹R²NH: dimethylamine |
| | HPLC: Basic conditions (Xselect OBD 19): Gradient 5-70% |
| | 2.1 mg, LCMS m/z = 338.2 [M + H]⁺ |
| 191 | N-(1′-(6-((2-methoxyethyl)amino)-4-methylpyridin-2-yl)- |
| | 1′,2′-dihydrospiro[cyclopropane- |
| | 1,3′-pyrrolo[3,2-c]pyridin]-6′-yl)acetamide |
| | 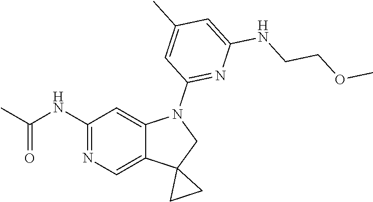 [SMILES: COCCNc1cc(C)cc(N2CC3(CC3)c3cnc(NC(C)=O)cc32)n1] |
| | R¹R²NH: 2-methoxyethylamine |
| | HPLC: Basic conditions (Xselect OBD 19): Gradient 5-60% |
| | 4.5 mg, LCMS m/z = 368.2 [M + H]⁺ |
| 192 | N-(1′-(6-(3-(dimethylamino)pyrrolidin-1-yl)-4- |
| | methylpyridin-2-yl)-1′,2′-dihydrospiro[cyclopropane- |
| | 1,3′-pyrrolo[3,2-c]pyridin]-6′-yl)acetamide |
| | trifluoroacetate |
| | 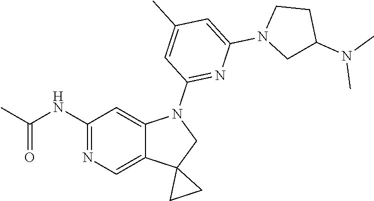 [SMILES: CC(=O)Nc1cc2c(cn1)C1(CC1)CN2c1cc(C)cc(N2CCC(N(C)C)C2)n1] |
| | R¹R²NH: 3-(dimethylamino)pyrrolidine |
| | HPLC: Acidic conditions (Sunfire OBD 19), Gradient 5-70% |
| | 2.3 mg, LCMS m/z = 407.3 [M + H]⁺ |
| 193 | N-(1′-(6-(3-hydroxyazetidin-1-yl)-4-methylpyridin-2-yl)-1′,2′- |
| | dihydrospiro[cyclopropane-1,3′- |
| | pyrrolo[3,2-c]pyridin]-6′-yl)acetamide OH |
| | 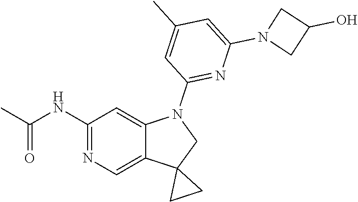 [SMILES: CC(=O)Nc1cc2c(cn1)C1(CC1)CN2c1cc(C)cc(N2CC(O)C2)n1] |
| | R¹R²NH: 3-hydroxyazetidine |
| | HPLC: Basic conditions (Xselect OBD 19): Gradient 5-55% |
| | 7.5 mg, LCMS m/z = 366.2 [M + H]⁺ |
| 194 | N-(1′-(6-(3-cyanoazetidin-1-yl)-4-methylpyridin-2-yl)-1′,2′- |
| | dihydrospiro[cyclopropane-1,3′-pyrrolo[3,2-c]pyridin]-6′-yl) |
| | acetamide |
| | 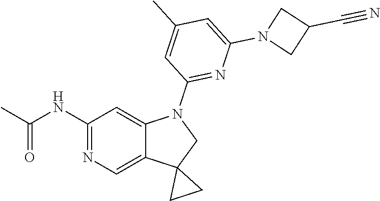 [SMILES: CC(=O)Nc1cc2c(cn1)C1(CC1)CN2c1cc(C)cc(N2CC(C#N)C2)n1] |
| | R¹R²NH: 3-cyanoazetidine |
| | HPLC: Basic conditions (Xselect OBD 19): Gradient 5-65% |
| | 14.9 mg, LCMS m/z = 375.2 [M + H]⁺ |
| 195 | N-(1′-(6-(3-fluoroazetidin-1-yl)-4-methylpyridin-2-yl)-1′,2′- |
| | dihydrospiro[cyclopropane-1,3′-pyrrolo[3,2-c]pyridin]-6′- |
| | yl)acetamide |
| | 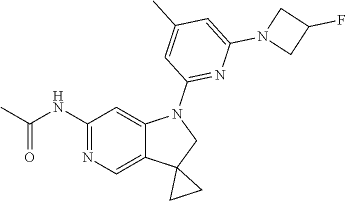 [SMILES: CC(=O)Nc1cc2c(cn1)C1(CC1)CN2c1cc(C)cc(N2CC(F)C2)n1] |
| | R¹R²NH: 3-fluoroazetidine |
| | HPLC: Basic conditions (Xselect OBD 19): Gradient 5-70% |
| | 15.8 mg, LCMS m/z = 368.2 [M + H]⁺ |
| 196 | N-(1′-(6-(3-cyano-3-(fluoromethyl)azetidin-1-yl)-4- |
| | methylpyridin-2-yl)-1′,2′-dihydrospiro |
| | [cyclopropane-1,3′-pyrrolo[3,2-c]pyridin]-6′-yl)acetamide |
| | 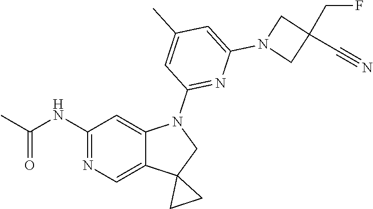 [SMILES: CC(=O)Nc1cc2c(cn1)C1(CC1)CN2c1cc(C)cc(N2CC(C#N)(CF)C2)n1] |
| | R¹R²NH: 3-cyano-3-(fluoromethyl)azetidine |
| | HPLC: Basic conditions (Xselect OBD 19): Gradient 5-65% |
| | 3.7 mg, LCMS m/z = 407.2 [M + H]⁺ |
| 197 | N-(1′-(6-(3-cyano-3-ethylazetidin-1-yl)-4-methylpyridin- |
| | 2-yl)-1′,2′-dihydrospiro[cyclopropane- |
| | 1,3′-pyrrolo[3,2-c]pyridin]-6′-yl)acetamide |
| | 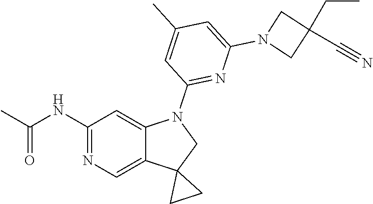 [SMILES: CCC1(C#N)CN(c2cc(C)cc(N3CC4(CC4)c4cnc(NC(C)=O)cc43)n2)C1] |
| | R¹R²NH: 3-cyano-3-ethylazetidine |
| | HPLC: Basic conditions (Xselect OBD 19): Gradient 5-70% |
| | 5.8 mg, LCMS m/z = 403.3 [M + H]⁺ |
| 198 | N-(1′-(6-(3-(difluoromethoxy)azetidin- |
| | 1-yl)-4-methylpyridin-2-yl)-1′,2′-dihydrospiro[cyclopropane- |
| | 1,3′-pyrrolo[3,2-c]pyridin]-6′-yl)acetamide |
| | 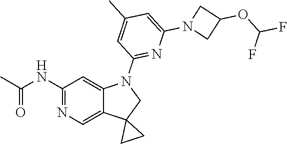 [SMILES: CC(=O)Nc1cc2c(cn1)C1(CC1)CN2c1cc(C)cc(N2CC(OC(F)F)C2)n1] |
| | R¹R²NH: 3-(difluoromethoxy)azetidine |
| | HPLC: Basic conditions (Xselect OBD 19): Gradient 10-70% |
| | 2.6 mg, LCMS m/z = 416.3 [M + H]⁺ |
| 199 | N-(1′-(6-(3-cyano-3-(2-methoxyethyl)azetidin-1-yl)- |
| | 4-methylpyridin-2-yl)-1′,2′-dihydrospiro |
| | [cyclopropane-1,3′-pyrrolo[3,2-c]pyridin]-6′-yl)acetamide |
| | 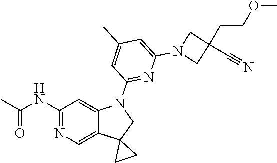 [SMILES: COCCC1(C#N)CN(c2cc(C)cc(N3CC4(CC4)c4cnc(NC(C)=O)cc43)n2)C1] |
| | R¹R²NH: 3-cyano-3-(2-methoxyethyl)azetidine |
| | HPLC: Basic conditions (Xselect OBD 19): Gradient 5-70% |
| | 7.2 mg, LCMS m/z = 433.3 [M + H]⁺ |
| 200 | N-(1′-(6-(3-cyano-3-methylazetidin-1-yl)-4-methylpyridin- |
| | 2-yl)-1′,2′-dihydrospiro[cyclopropane- |
| | 1,3′-pyrrolo[3,2-c]pyridin]-6′-yl)acetamide |
| | 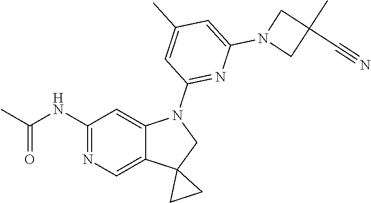 [SMILES: CC(=O)Nc1cc2c(cn1)C1(CC1)CN2c1cc(C)cc(N2CC(C)(C#N)C2)n1] |
| | R¹R²NH: 3-cyano-3-methylazetidine |
| | HPLC: Basic conditions (Xselect OBD 19): Gradient 5-70% |
| | 3.9 mg, LCMS m/z = 389.3 [M + H]⁺ |
| 201 | N-(1′-(6-(3-(difluoromethyl)azetidin-1-yl)-4-methylpyridin- |
| | 2-yl)-1′,2′-dihydrospiro[cyclopropane- |
| | 1,3′-pyrrolo[3,2-c]pyridin]-6′-yl)acetamide |
| | trifluoroacetate |
| | 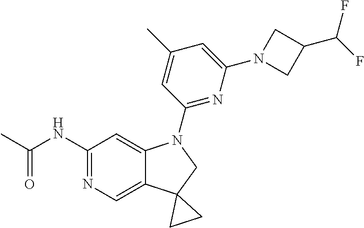 [SMILES: CC(=O)Nc1cc2c(cn1)C1(CC1)CN2c1cc(C)cc(N2CC(C(F)F)C2)n1] |
| | R¹R²NH: 3-(difluoromethyl)azetidine |
| | HPLC: Acidic conditions (Sunfire OBD 19), Gradient 5-70% |
| | 11.6 mg, LCMS m/z = 400.3 [M + H]⁺ |
| 202 | N-(1′-(6-(3-cyano-3-(difluoromethyl)azetidin-1-yl)- |
| | 4-methylpyridin-2-yl)-1′,2′-dihydrospiro |
| | [cyclopropane-1,3′-pyrrolo[3,2-c]pyridin]-6′-yl)acetamide |
| | 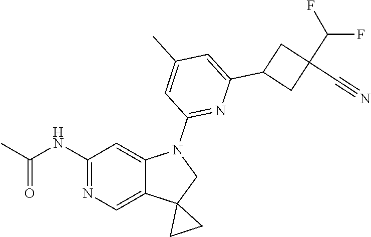 [SMILES: CC(=O)Nc1cc2c(cn1)C1(CC1)CN2c1cc(C)cc(C2CC(C#N)(C(F)F)C2)n1] |
| | R¹R²NH: 3-cyano-3-(difluoromethyl)azetidine |
| | HPLC: Basic conditions (Xselect OBD 19): Gradient 5-70% |
| | 8.0 mgs, LCMS m/z = 425.3 [M + H]⁺ |
| 203 | N-(1′-(4-methyl-6-(3-(methylsulfonyl)azetidin-1-yl) |
| | pyridin-2-yl)-1′,2′-dihydrospiro |
| | [cyclopropane-1,3′-pyrrolo[3,2-c]pyridin]-6′-yl)acetamide |
| | 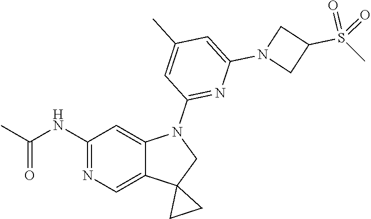 [SMILES: CC(=O)Nc1cc2c(cn1)C1(CC1)CN2c1cc(C)cc(N2CC(S(C)(=O)=O)C2)n1] |
| | R¹R²NH: 3-(methylsulfonyl)azetidine |
| | HPLC: Basic conditions (Xselect OBD 19): Gradient 5-60% |
| | 5.7 mgs, LCMS m/z = 428.2 [M + H]⁺ |
| 204 | N-(1′-(6-(3-(ethylsulfonyl)azetidin-1-yl)-4- |
| | methylpyridin-2-yl)-1′,2′-dihydrospiro |
| | [cyclopropane-1,3′-pyrrolo[3,2-c]pyridin]-6′-yl)acetamide |
| | 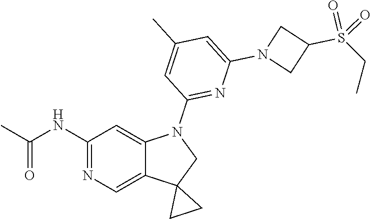 [SMILES: CCS(=O)(=O)C1CN(c2cc(C)cc(N3CC4(CC4)c4cnc(NC(C)=O)cc43)n2)C1] |
| | R¹R²NH: 3-(ethylsulfonyl)azetidine |
| | HPLC: Basic conditions (Xselect OBD 19): Gradient 5-60% |
| | 3.8 mgs, LCMS m/z = 442.2 [M + H]⁺ |
| 205 | N-(1′-(6-(3-hydroxy-3-methylazetidin-1-yl)-4- |
| | methylpyridin-2-yl)-1′,2′-dihydrospiro[cyclopropane- |
| | 1,3′-pyrrolo[3,2-c]pyridin]-6′-yl)acetamide |
| | 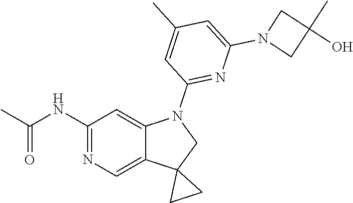 [SMILES: CC(=O)Nc1cc2c(cn1)C1(CC1)CN2c1cc(C)cc(N2CC(C)(O)C2)n1] |
| | R¹R²NH: 3-hydroxy-3-methylazetidine |
| | HPLC: Basic conditions (Xselect OBD 19): Gradient 5-60% |
| | 3.2 mg, LCMS m/z = 380.2 [M + H]⁺ |
| 206 | N-(1′-(4-methyl-6-morpholinopyridin-2-yl)-1′,2′- |
| | dihydrospiro[cyclopropane-1,3′-pyrrolo[3,2-c]pyridin]- |
| | 6′-yl)acetamide |
| | 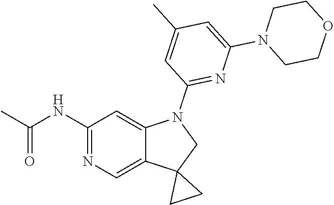 [SMILES: CC(=O)Nc1cc2c(cn1)C1(CC1)CN2c1cc(C)cc(N2CCOCC2)n1] |
| | R¹R²NH: morpholine |
| | HPLC: Basic conditions (Xselect OBD 19): Gradient 5-65% |
| | 7.8 mg, LCMS m/z = 380.2 [M + H]⁺ |
| 207 | N-(1′-(4-methyl-6-(4-methylpiperazin-1-yl)pyridin-2-yl)-1′,2′- |
| | dihydrospiro[cyclopropane-1,3′-pyrrolo[3,2-c]pyridin]- |
| | 6′-yl)acetamide |
| | 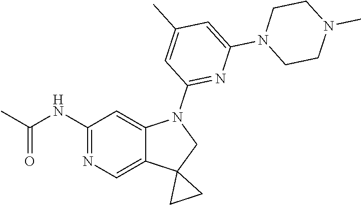 [SMILES: CC(=O)Nc1cc2c(cn1)C1(CC1)CN2c1cc(C)cc(N2CCN(C)CC2)n1] |
| | R¹R²NH: 4-methylpiperazine |
| | HPLC: Basic conditions (OBD 19): Gradient 5-65% |
| | 7.7 mg, LCMS m/z = 393.3 [M + H]⁺ |
| 208 | N-(1′-(4-methyl-6-(2-oxa-7-azaspiro[3.5]nonan-7-yl) |
| | pyridin-2-yl)-1′,2′-dihydrospiro[cyclopropane- |
| | 1,3′-pyrrolo[3,2-c]pyridin]-6′-yl)acetamide |
| | 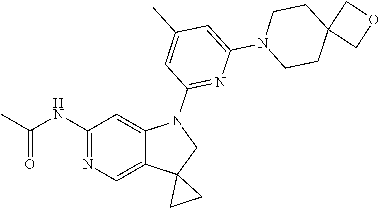 [SMILES: CC(=O)Nc1cc2c(cn1)C1(CC1)CN2c1cc(C)cc(N2CCC3(CC2)COC3)n1] |
| | R¹R²NH: 2-oxa-7-azaspiro[3.5]nonane |
| | HPLC: Basic conditions (Xselect OBD 19): Gradient 5-65% |
| | 6.0 mg, LCMS m/z = 420.3 [M + H]⁺ |

### Examples 209 to 212

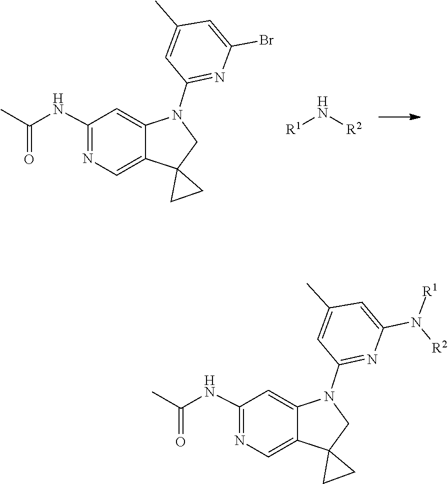

An 8 mL vial was charged with N-(1′-(6-bromo-4-methylpyridin-2-yl)-1′,2′-dihydrospiro[cyclopropane-1,3′-pyrrolo[3,2-c]pyridin]-6′-yl)acetamide (Preparation 157, 28.0 mg, 75 μmol), R¹R²NH (75 μmol), Xantphos Pd G3 (7.1 mg, 7.5 μmol), Xantphos (4.3 mg, 7.5 μmol) and NaOtBu (21.6 mg, 225 μmol). Dioxane (1.5 mL, 0.2 M) was added under inert atmosphere. The vial was sealed with a Teflon cap, and the reaction solution was heated under reflux at 90° C. for 16 h. The cooled reaction mixture was diluted with EtOAc, washed with H₂O (3 mL), and extracted with EtOAc (5 mL, 2×). The combined organic phases were dried over anhydrous MgSO₄ and concentrated. The crude material was dissolved in DCM (1.5 mL, 0.2 M) and TFA (17.2 μL, 225 μmol) was added to the reaction solution. The reaction mixture was stirred at room temp for 16 h. The reaction mixture was then diluted with DCM (3 mL) and washed with sat. aq. NaHCO₃. The layers were separated, and the aqueous phase was extracted with DCM (5 mL, 2×). The combined organic phases were dried over Na₂SO₄ and concentrated in vacuo to give the crude product. This was purified by HPLC using a Waters SunFire Prep C18 column (5 um OBD 30×100 mm) under acidic conditions.
Acidic conditions (Sunfire OBD 30): MeCN, water, 0.1% TFA modifier

| Example No | Name, Structure, R¹R²NH, HPLC, Data |
|---|---|
| 209 | N-(1′-(6-((3aR,6aS)-hexahydropyrrolo[3,4-c]pyrrol-2(1H)-yl)-4- methylpyridin-2-yl)-1′,2′-dihydrospiro[cyclopropane-1,3′-pyrrolo[3,2- c]pyridin]-6′-yl)acetamide trifluoroacetate 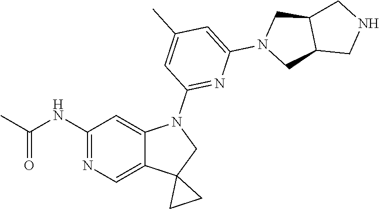 [SMILES: CC(=O)Nc1cc2c(cn1)C1(CC1)CN2c1cc(C)cc(N2C[C@H]3CNC[C@H]3C2)n1] R¹R²NH: tert-butyl (3aR,6aS)-hexahydropyrrolo[3,4-c]pyrrole-2(1H)- carboxylate. HPLC: Acidic conditions (Sunfire OBD 30): Gradient 5-65% 11. mg, LCMS m/z 405.3 [M + H]⁺ |
| 210 | N-(1′-(6-(hexahydropyrrolo[3,4-b]pyrrol-5(1H)-yl)-4-methylpyridin-2- yl)-1′,2′-dihydrospiro[cyclopropane-1,3′-pyrrolo[3,2-c]pyridin]-6′- yl)acetamide trifluoroacetate 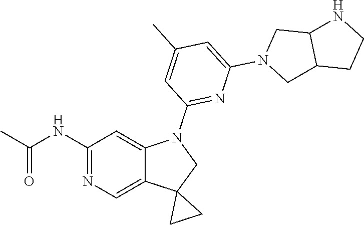 [SMILES: CC(=O)Nc1cc2c(cn1)C1(CC1)CN2c1cc(C)cc(N2CC3CCNC3C2)n1] R¹R²NH: octahydropyrrolo[3,4-b]pyrrole HPLC: Acidic conditions (Sunfire OBD 30): Gradient 5-65% 4.80 mg, LCMS m/z = 405.3 [M + H]⁺ |
| 211 | N-(1′-(4-methyl-6-(piperazin-1-yl)pyridin-2-yl)-1′,2′- dihydrospiro[cyclopropane-1,3′-pyrrolo[3,2-c]pyridin]-6′-yl)acetamide trifluoroacetate 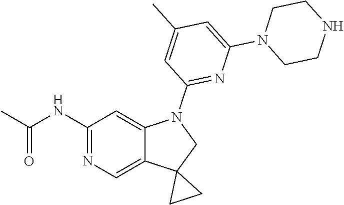 [SMILES: CC(=O)Nc1cc2c(cn1)C1(CC1)CN2c1cc(C)cc(N2CCNCC2)n1] R¹R²NH: tert-butyl piperazine-1-carboxylate HPLC: Acidic conditions (Sunfire OBD 30): Gradient 5-65% 15.4 mg, LCMS m/z = 379.3 [M + H]⁺ |
| 212 | N-(1′-(6-(3-(2-hydroxyethyl)piperazin-1-yl)-4-methylpyridin-2-yl)-1′,2′- dihydrospiro[cyclopropane-1,3′-pyrrolo[3,2-c]pyridin]-6′-yl)acetamide trifluoroacetate 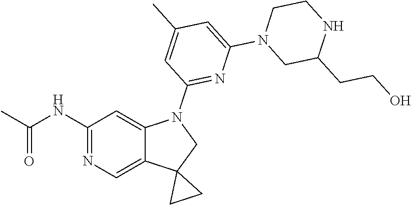 [SMILES: CC(=O)Nc1cc2c(cn1)C1(CC1)CN2c1cc(C)cc(N2CCNC(CCO)C2)n1] R¹R²NH: tert-butyl 2-(2-hydroxyethyl)piperazine-1-carboxylate HPLC: Acidic conditions (Sunfire OBD 30): Gradient 5-60% 3.6 mg, LCMS m/z = 423.3 [M + H]⁺ |

### Examples 213 to 237

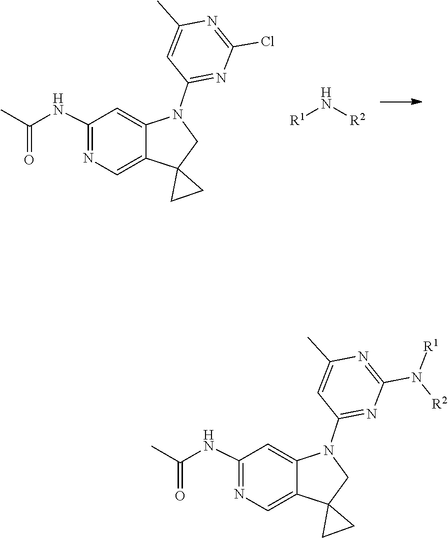

An 8 mL vial was charged with N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 23.0 mg, 100 μmol), R¹R²NH (100 μmol), Xantphos Pd G3 (9.5 mg, 10 μmol), Xantphos (5.8 mg, 10 μmol) and NaOtBu (28.8 mg, 300 μmol). Dioxane (2.0 mL, 0.2 M) was added under inert atmosphere. The vial was sealed with a Teflon cap, and the reaction solution was heated under reflux at 90° C. for 16 h. The cooled reaction mixture was diluted with EtOAc, washed with H₂O (3 mL), and extracted with EtOAc (5 mL, 2×). The combined organic phases were dried over anhydrous MgSO₄ and concentrated in vacuo to leave the crude product. This was purified by HPLC using a Waters SunFire Prep C18 column (5 um OBD 19×100 mm) under acidic conditions, or a Waters XSelect CSH Prep C18 column (5 um OBD 19×100 mm) under basic conditions.
Basic conditions (Xselect OBD 19): MeCN, water, 0.2% NH₄OH modifier
Acidic conditions (Sunfire OBD 19): MeCN, water, 0.1% TFA modifier

| Example | |
|---|---|
| No | Name, Structure, R¹R²NH, HPLC, Data |
| 213 | N-(1′-(6-methyl-2-morpholinopyrimidin-4-yl)-1′,2′- dihydrospiro[cyclopropane-1,3′-pyrrolo[3,2-c]pyridin]-6′-yl)acetamide 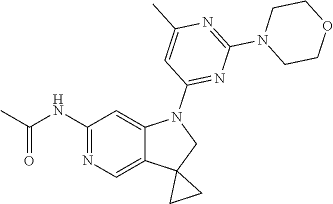 [SMILES: CC(=O)Nc1cc2c(cn1)C1(CC1)CN2c1cc(C)nc(N2CCOCC2)n1] R¹R²NH: morpholine HPLC: Basic conditions (Xselect OBD 19): Gradient 5-55% 2.0 mg, LCMS m/z = 381.2 [M + H]⁺ |
| 214 | N-(1′-(2-(3-hydroxypyrrolidin-1-yl)-6-methylpyrimidin-4-yl)-1′,2′- dihydrospiro[cyclopropane-1,3′-pyrrolo[3,2-c]pyridin]-6′-yl)acetamide 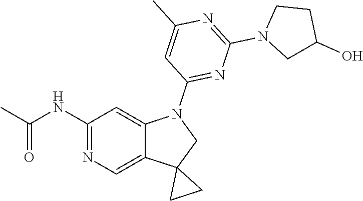 [SMILES: CC(=O)Nc1cc2c(cn1)C1(CC1)CN2c1cc(C)nc(N2CCC(O)C2)n1] R¹R²NH: 3-hydroxypyrrolidine HPLC: Basic conditions (Xselect OBD 19): Gradient 5-60% 2.3 mg, LCMS m/z = 381.2 [M + H]⁺ |
| 215 | N-(1′-(2-(3-(dimethylamino)pyrrolidin-1-yl)-6-methylpyrimidin-4-yl)-1′,2′- dihydrospiro[cyclopropane-1,3′-pyrrolo[3,2-c]pyridin]-6′-yl)acetamide 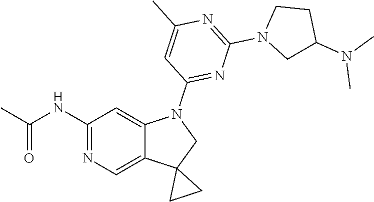 [SMILES: CC(=O)Nc1cc2c(cn1)C1(CC1)CN2c1cc(C)nc(N2CCC(N(C)C)C2)n1] R¹R²NH: 3-(dimethylamino)pyrrolidine HPLC: Basic conditions (Xselect OBD 19): Gradient 5-60% 2.5 mg, LCMS m/z = 408.3 [M + H]⁺; |
| 216 | N-(1′-(2-(3-cyanoazetidin-1-yl)-6-methylpyrimidin-4-yl)-1′,2′- dihydrospiro[cyclopropane-1,3′-pyrrolo[3,2-c]pyridin]-6′-yl)acetamide 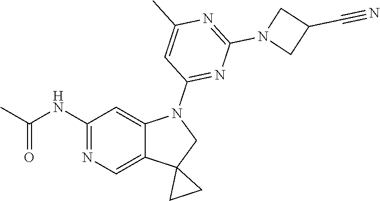 [SMILES: CC(=O)Nc1cc2c(cn1)C1(CC1)CN2c1cc(C)nc(N2CC(C#N)C2)n1] R¹R²NH: 3-cyanoazetidine HPLC: Basic conditions (Xselect OBD 19): Gradient 5-50% 1.4 mg, LCMS m/z = 376.2 [M + H]⁺ |
| 217 | N-(1′-(2-(3-fluoroazetidin-1-yl)-6-methylpyrimidin-4-yl)-1′,2′- dihydrospiro[cyclopropane-1,3′-pyrrolo[3,2-c]pyridin]-6′-yl)acetamide 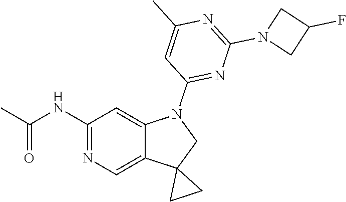 [SMILES: CC(=O)Nc1cc2c(cn1)C1(CC1)CN2c1cc(C)nc(N2CC(F)C2)n1] R¹R²NH: 3-fluoroazetidine HPLC: Basic conditions (Xselect OBD 19): Gradient 5-55% 4.6 mg, LCMS m/z = 369.2 [M + H]⁺ |
| 218 | N-(1′-(2-(3-cyano-3-(fluoromethyl)azetidin-1-yl)-6-methylpyrimidin-4-yl)- 1′,2′-dihydrospiro[cyclopropane-1,3′-pyrrolo[3,2-c]pyridin]-6′-yl)acetamide 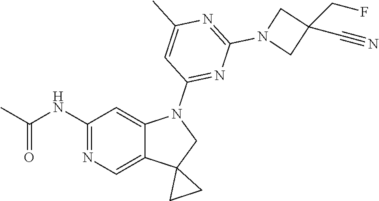 [SMILES: CC(=O)Nc1cc2c(cn1)C1(CC1)CN2c1cc(C)nc(N2CC(C#N)(CF)C2)n1] R¹R²NH: 3-cyano-3-(fluoromethyl)azetidine HPLC: Basic conditions (Xselect OBD 19): Gradient 5-55% 1.5 mg, LCMS m/z = 408.2 [M + H]⁺ |
| 219 | N-(1′-(2-(3-cyano-3-ethylazetidin-1-yl)-6-methylpyrimidin-4-yl)-1′,2′- dihydrospiro[cyclopropane-1,3′-pyrrolo[3,2-c]pyridin]-6′-yl)acetamide 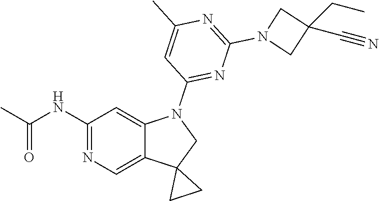 [SMILES: CCC1(C#N)CN(c2nc(C)cc(N3CC4(CC4)c4cnc(NC(C)=O)cc43)n2)C1] R¹R²NH: 3-cyano-3-ethylazetidine HPLC: Basic conditions (Xselect OBD 19): Gradient 5-60% 4.4 mg, LCMS m/z = 404.3 [M + H]⁺ |
| 220 | N-(1′-(2-(3-cyano-3-(2-methoxyethyl)azetidin-1-yl)-6-methylpyrimidin-4-yl)- 1′,2′-dihydrospiro[cyclopropane-1,3′-pyrrolo[3,2-c]pyridin]-6′-yl)acetamide 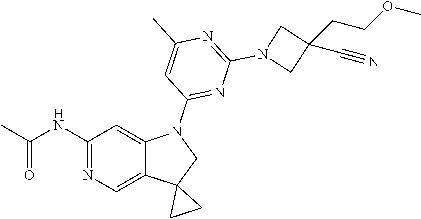 [SMILES: COCCC1(C#N)CN(c2nc(C)cc(N3CC4(CC4)c4cnc(NC(C)=O)cc43)n2)C1] R¹R²NH: 3-cyano-3-(2-methoxyethyl)azetidine HPLC: Basic conditions (Xselect OBD 19): Gradient 5-60% 12.8 mg, LCMS m/z = 434.3 [M + H]⁺ |
| 221 | N-(1′-(2-(3-cyano-3-methylazetidin-1-yl)-6-methylpyrimidin-4-yl)-1′,2′- dihydrospiro[cyclopropane-1,3′-pyrrolo[3,2-c]pyridin]-6′-yl)acetamide 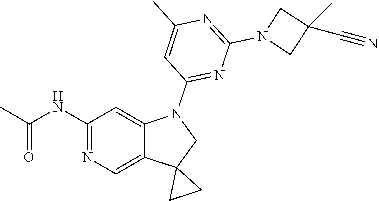 [SMILES: CC(=O)Nc1cc2c(cn1)C1(CC1)CN2c1cc(C)nc(N2CC(C)(C#N)C2)n1] R¹R²NH: 3-cyano-3-methylazetidine HPLC: Basic conditions (Xselect OBD 19): Gradient 5-55% 5.7 mg, LCMS m/z = 390.2 [M + H]⁺ |
| 222 | N-(1′-(2-(3-(difluoromethyl)azetidin-1-yl)-6-methylpyrimidin-4-yl)-1′,2′- dihydrospiro[cyclopropane-1,3′-pyrrolo[3,2-c]pyridin]-6′-yl)acetamide 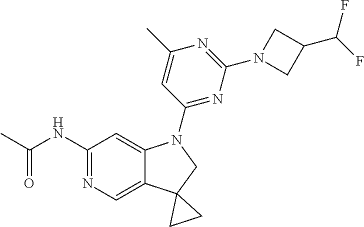 [SMILES: CC(=O)Nc1cc2c(cn1)C1(CC1)CN2c1cc(C)nc(N2CC(C(F)F)C2)n1] R¹R²NH: 3-(difluoromethyl)azetidine HPLC: Basic conditions (Xselect OBD 19): Gradient 5-55% 6.7 mg, LCMS m/z = 401.2 [M + H]⁺ |
| 223 | N-(1′-(2-(3-cyano-3-(difluoromethyl)azetidin-1-yl)-6-methylpyrimidin-4-yl)- 1′,2′-dihydrospiro[cyclopropane-1,3′-pyrrolo[3,2-c]pyridin]-6′-yl)acetamide trifluoroacetate 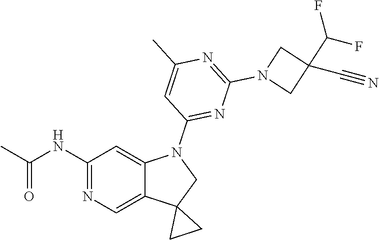 [SMILES: CC(=O)Nc1cc2c(cn1)C1(CC1)CN2c1cc(C)nc(N2CC(C#N)(C(F)F)C2)n1] R¹R²NH: 3-cyano-3-(difluoromethyl)azetidine HPLC: Acidic conditions (Xselect OBD 19): Gradient 5-60% 2.0 mg, LCMS m/z = 426.2 [M + H]⁺ |
| 224 | N-(1′-(6-methyl-2-(3-(methylsulfonyl)azetidin-1-yl)pyrimidin-4-yl)-1′,2′- dihydrospiro[cyclopropane-1,3′-pyrrolo[3,2-c]pyridin]-6′-yl)acetamide 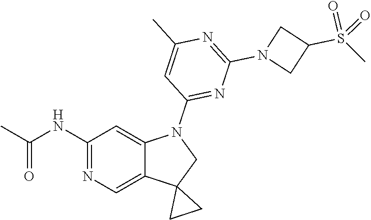 [SMILES: CC(=O)Nc1cc2c(cn1)C1(CC1)CN2c1cc(C)nc(N2CC(S(C)(=O)=O)C2)n1] R¹R²NH: 3-(methylsulfonyl)azetidine HPLC: Basic conditions (Xselect OBD 19): Gradient 5-45% 4.3 mg, LCMS m/z = 429.2 [M + H]⁺ |
| 225 | N-(1′-(2-(3-(ethylsulfonyl)azetidin-1-yl)-6-methylpyrimidin-4-yl)-1′,2′- dihydrospiro[cyclopropane-1,3′-pyrrolo[3,2-c]pyridin]-6′-yl)acetamide 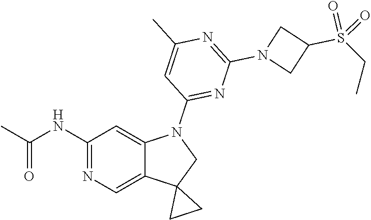 [SMILES: CCS(=O)(=O)C1CN(c2nc(C)cc(N3CC4(CC4)c4cnc(NC(C)=O)cc43)n2)C1] R¹R²NH: 3-(ethylsulfonyl)azetidine HPLC: Basic conditions (Xselect OBD 19): Gradient 5-50% 2.8 mg, LCMS m/z = 443.2 [M + H]⁺ |
| 226 | N-(1′-(2-(3-hydroxy-3-methylazetidin-1-yl)-6-methylpyrimidin-4-yl)-1′,2′- dihydrospiro[cyclopropane-1,3′-pyrrolo[3,2-c]pyridin]-6′-yl)acetamide 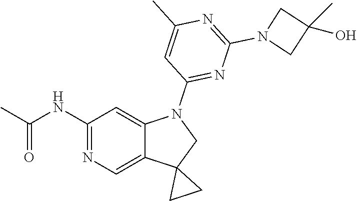 [SMILES: CC(=O)Nc1cc2c(cn1)C1(CC1)CN2c1cc(C)nc(N2CC(C)(O)C2)n1] R¹R²NH: 3-hydroxy-3-methylazetidine HPLC: Basic conditions (Xselect OBD 19): Gradient 5-45% 9.5 mg, LCMS m/z = 381.2 [M + H]⁺ |
| 227 | N-(1′-(6-methyl-2-(4-methylpiperazin-1-yl)pyrimidin-4-yl)-1′,2′- dihydrospiro[cyclopropane-1,3′-pyrrolo[3,2-c]pyridin]-6′-yl)acetamide 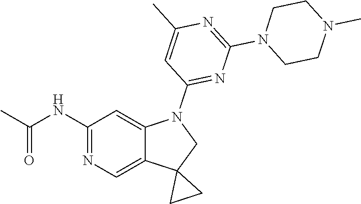 [SMILES: CC(=O)Nc1cc2c(cn1)C1(CC1)CN2c1cc(C)nc(N2CCN(C)CC2)n1] R¹R²NH: 4-methylpiperazine HPLC: Basic conditions (Xselect OBD 19): Gradient 5-55% 2.5 mg, LCMS m/z = 394.3 [M + H]⁺ |
| 228 | N-(1′-(6-methyl-2-(2-oxa-7-azaspiro[3.5]nonan-7-yl)pyrimidin-4-yl)-1′,2′- dihydrospiro[cyclopropane-1,3′-pyrrolo[3,2-c]pyridin]-6′-yl)acetamide trifluoroacetate 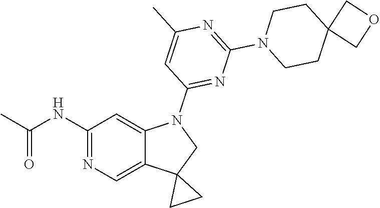 [SMILES: CC(=O)Nc1cc2c(cn1)C1(CC1)CN2c1cc(C)nc(N2CCC3(CC2)COC3)n1] R¹R²NH: 2-oxa-7-azaspiro[3.5]nonane HPLC: Acidic conditions (SunFire OBD 19); Gradient 5-60% 2.2 mg, LCMS m/z = 421.3 [M + H]⁺ |
| 229 | N-(1′-(2-(3,3-difluoropyrrolidin-1-yl)-6-methylpyrimidin-4-yl)-1′,2′- dihydrospiro[cyclopropane-1,3′-pyrrolo[3,2-c]pyridin]-6′-yl)acetamide 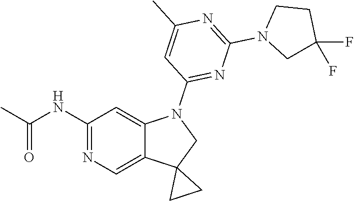 [SMILES: CC(=O)Nc1cc2c(cn1)C1(CC1)CN2c1cc(C)nc(N2CCC(F)(F)C2)n1] R¹R²NH: 3,3-difluoropyrrolidine HPLC: Basic conditions (Xselect OBD 19): Gradient 5-65% 3.9 mg, LCMS m/z = 401.2 [M + H]⁺ |
| 230 | N-(1′-(2-(3-methoxypiperidin-1-yl)-6-methylpyrimidin-4-yl)-1′,2′- dihydrospiro[cyclopropane-1,3′-pyrrolo[3,2-c]pyridin]-6′-yl)acetamide 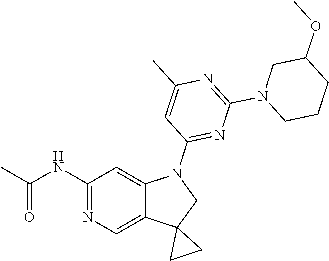 [SMILES: COC1CCCN(c2nc(C)cc(N3CC4(CC4)c4cnc(NC(C)=O)cc43)n2)C1] R¹R²NH: 3-methoxypiperidine HPLC: Basic conditions (Xselect OBD 19): Gradient 5-65% 12.5 mg, LCMS m/z = 409.3 [M + H]⁺ |
| 231 | N-(1′-(2-(4-(cyclopropanecarbonyl)-4-methoxypiperidin-1-yl)-6- methylpyrimidin-4-yl)-1′,2′-dihydrospiro[cyclopropane-1,3′-pyrrolo[3,2- c]pyridin]-6′-yl)acetamide trifluoroacetate 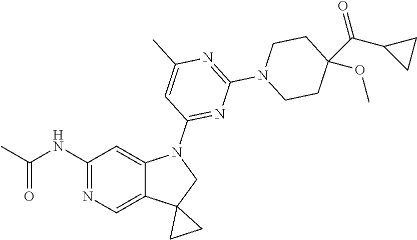 [SMILES: COC1(C(=O)C2CC2)CCN(c2nc(C)cc(N3CC4(CC4)c4cnc(NC(C)=O)cc43)n2)CC1] R¹R²NH: cyclopropyl(4-methoxypiperidin-4-yl)methanone HPLC: Acidic conditions (SunFire OBD 19); Gradient 10-70% 1.3 mg, LCMS m/z = 477.3 [M + H]⁺ |
| 232 | N-(1′-(2-(4-hydroxypiperidin-1-yl)-6-methylpyrimidin-4-yl)-1′,2′- dihydrospiro[cyclopropane-1,3′-pyrrolo[3,2-c]pyridin]-6′-yl)acetamide 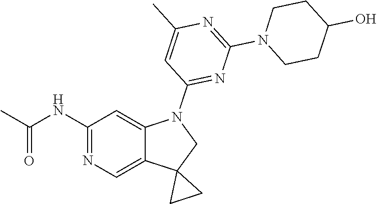 [SMILES: CC(=O)Nc1cc2c(cn1)C1(CC1)CN2c1cc(C)nc(N2CCC(O)CC2)n1] R¹R²NH: 4-hydroxypiperidine HPLC: Basic conditions (Xselect OBD 19): Gradient 5-55% 1.2 mg, LCMS m/z = 395.3 [M + H]⁺ |
| 233 | N-(1′-(2-(4-(2-methoxyethyl)piperazin-1-yl)-6-methylpyrimidin-4-yl)-1′,2′- dihydrospiro[cyclopropane-1,3′-pyrrolo[3,2-c]pyridin]-6′-yl)acetamide trifluoroacetate 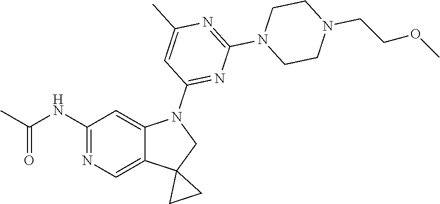 [SMILES: COCCN1CCN(c2nc(C)cc(N3CC4(CC4)c4cnc(NC(C)=O)cc43)n2)CC1] R¹R²NH: 4-(2-methoxyethyl)piperazine HPLC: Acidic conditions (SunFire OBD 19); Gradient 5-60% 0.9 mg, LCMS m/z = 438.3 [M + H]⁺ |
| 234 | N-(1′-(2-(3-methoxy-3-methylazetidin-1-yl)-6-methylpyrimidin-4-yl)-1′,2′- dihydrospiro[cyclopropane-1,3′-pyrrolo[3,2-c]pyridin]-6′-yl)acetamide 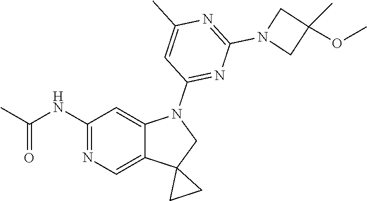 [SMILES: COC1(C)CN(c2nc(C)cc(N3CC4(CC4)c4cnc(NC(C)=O)cc43)n2)C1] R¹R²NH: 3-methoxy-3-methylazetidine HPLC: Basic conditions (Xselect OBD 19): Gradient 5-55% 12.0 mg, LCMS m/z = 395.3 [M + H]⁺ |
| 235 | N-(1′-(6-methyl-2-(2-methylazetidin-1-yl)pyrimidin-4-yl)-1′,2′- dihydrospiro[cyclopropane-1,3′-pyrrolo[3,2-c]pyridin]-6′-yl)acetamide 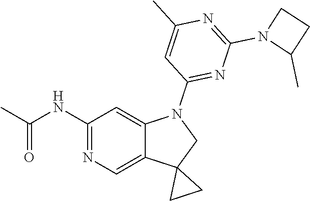 [SMILES: CC(=O)Nc1cc2c(cn1)C1(CC1)CN2c1cc(C)nc(N2CCC2C)n1] R¹R²NH: 2-methylazetidine HPLC: Basic conditions (Xselect OBD 19): Gradient 5-60% 25.6 mg, LCMS m/z = 365.3 [M + H]⁺ |
| 236 | N-(1′-(2-(3-hydroxy-3-methylpyrrolidin-1-yl)-6-methylpyrimidin-4-yl)-1′,2′- dihydrospiro[cyclopropane-1,3′-pyrrolo[3,2-c]pyridin]-6′-yl)acetamide 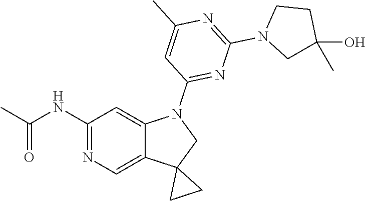 [SMILES: CC(=O)Nc1cc2c(cn1)C1(CC1)CN2c1cc(C)nc(N2CCC(C)(O)C2)n1] R¹R²NH: 3-hydroxy-3-methylpyrrolidine HPLC: Basic conditions (Xselect OBD 19): Gradient 5-55% 0.6 mg, LCMS m/z = 395.3 [M + H]⁺ |
| 237 | N-(1′-(2-(3-hydroxyazetidin-1-yl)-6-methylpyrimidin-4-yl)-1′,2′- dihydrospiro[cyclopropane-1,3′-pyrrolo[3,2-c]pyridin]-6′-yl)acetamide 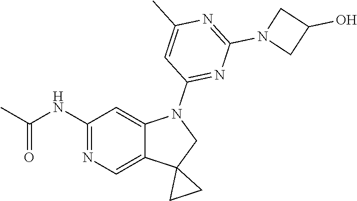 [SMILES: CC(=O)Nc1cc2c(cn1)C1(CC1)CN2c1cc(C)nc(N2CC(O)C2)n1] R¹R²NH: 3-hydroxyazetidine HPLC: Basic conditions (Xselect OBD 19): Gradient 5-50% 1.4 mg, LCMS m/z = 367.2 [M + H]⁺ |

### Example 238

### N-(1′-(2-(1,1-difluoroethyl)-6-(1-methyl-1H-pyrazol-4-yl)pyrimidin-4-yl)-1′,2′-dihydrospiro[cyclopropane-1,3′-pyrrolo[3,2-c]pyridin]-6′-yl)acetamide

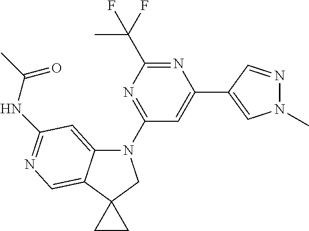 [SMILES: CC(=O)Nc1cc2c(cn1)C1(CC1)CN2c1cc(-c2cnn(C)c2)nc(C(C)(F)F)n1]

A mixture of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 40 mg, 0.105 mmol), 1-methyl-4-(4,4,5,5-tetramethyl-1,3,2-dioxaborolan-2-yl)pyrazole (45 mg, 0.216 mmol), K₂CO₃ (35 mg, 0.253 mmol) and Pd(dppf)Cl₂ (5 mg, 6.8 μmol) in dioxane (2 mL) and water (1 mL) was stirred at 95° C. for 1 h. The reaction was diluted with water (10 mL) and extracted with EtOAc (3×15 mL). The combined organic layers were concentrated to dryness, dissolved in DMSO, filtered through a syringe filter, and purified by reverse phase prep-HPLC to give N-(1′-(2-(1,1-difluoroethyl)-6-(1-methyl-1H-pyrazol-4-yl)pyrimidin-4-yl)-1′,2′-dihydrospiro[cyclopropane-1,3′-pyrrolo[3,2-c]pyridin]-6′-yl)acetamide (16.5 mg, 36.8% yield). LCMS m/z=426.4 [M+H]⁺. ¹H NMR (DMSO-d₆) δ: 10.31 (s, 1H), 9.03 (br s, 1H), 8.49 (s, 1H), 8.19 (s, 1H), 7.72 (s, 1H), 7.11 (s, 1H), 4.24 (s, 2H), 3.92 (s, 3H), 2.19 (s, 3H), 2.09 (s, 3H), 1.29-1.20 (m, 2H), 1.20-1.15 (m, 2H).

### Example 239 to 244

The compounds in the following table were prepared from N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155) and the appropriate boronate ester, following the procedure described in Example 238.

| Example | |
|---|---|
| No | Name, Structure, Boronate Ester, Data |
| 239^{A} | N-(1′-(2-(1,1-difluoroethyl)-6-(1,3-dimethyl-1H-pyrazol-4-yl)pyrimidin-4- yl)-1′,2′-dihydrospiro[cyclopropane-1,3′-pyrrolo[3,2-c]pyridin]-6′- yl)acetamide trifluoroacetate 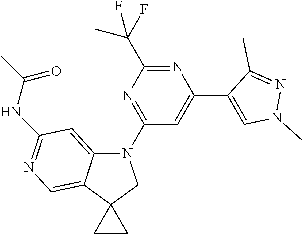 [SMILES: CC(=O)Nc1cc2c(cn1)C1(CC1)CN2c1cc(-c2cn(C)nc2C)nc(C(C)(F)F)n1] Boronate: 1,3-dimethyl-4-(4,4,5,5-tetramethyl-1,3,2-dioxaborolan-2- yl)pyrazole 17.3 mg, 37.4% yield. LCMS m/z = 440.3 [M + H]⁺. ¹H NMR (DMSO-d₆) δ: 11.44-10.38 (m, 1H), 8.86-8.57 (m, 1H), 8.50 (s, 1H), 7.71 (s, 1H), 7.05 (s, 1H), 4.31 (s, 2H), 3.84 (s, 3H), 2.17 (m, 6H), 1.27 (br s, 2H), 1.24 (br s, 2H). |
| 240^{A} | N-(1′-(2-(1,1-difluoroethyl)-6-(1,5-dimethyl-1H-pyrazol-4-yl)pyrimidin-4- yl)-1′,2′-dihydrospiro[cyclopropane-1,3′-pyrrolo[3,2-c]pyridin]-6′- yl)acetamide trifluoroacetate 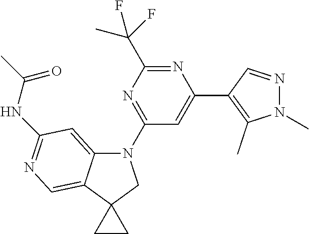 [SMILES: CC(=O)Nc1cc2c(cn1)C1(CC1)CN2c1cc(-c2cnn(C)c2C)nc(C(C)(F)F)n1] Boronate: 1,5-dimethyl-4-(4,4,5,5-tetramethyl-1,3,2-dioxaborolan-2- yl)pyrazole 18.4 mg, 39.8% yield. LCMS m/z = 440.3 [M + H]⁺. ¹H NMR (DMSO-d₆) δ: 11.54-10.19 (m, 1H), 9.13-8.40 (m, 1H), 8.18 (s, 1H), 7.75-7.62 (m, 1H), 7.12 (s, 1H), 4.32 (s, 2H), 3.81 (s, 3H), 2.71 (s, 3H), 2.25-2.09 (m, 6H), 1.30-1.24 (m, 2H), 1.24-1.19 (m, 2H). |
| 241 | N-(1′-(2-(1,1-difluoroethyl)-6-(1-methyl-1H-pyrazol-3-yl)pyrimidin-4-yl)- 1′,2′-dihydrospiro[cyclopropane-1,3′-pyrrolo[3,2-c]pyridin]-6′-yl)acetamide 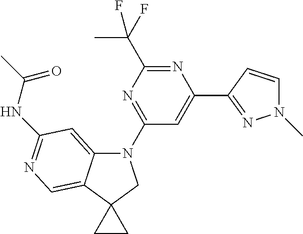 [SMILES: CC(=O)Nc1cc2c(cn1)C1(CC1)CN2c1cc(-c2ccn(C)n2)nc(C(C)(F)F)n1] Boronate: 1-methyl-3-(4,4,5,5-tetramethyl-1,3,2-dioxaborolan-2- yl)pyrazole. 4.5 mg, 10% yield. LCMS m/z = 426.4 [M + H]⁺. ¹H NMR (DMSO-d₆) δ: 10.31 (s, 1H), 9.06 (s, 1H), 7.87 (d, J = 2.4 Hz, 1H), 7.72 (s, 1H), 7.23 (s, 1H), 6.93 (d, J = 2.4 Hz, 1H), 4.27 (s, 2H), 3.96 (s, 3H), 2.28-2.15 (m, 3H), 2.09 (s, 3H), 1.23 (s, 2H), 1.18 (s, 2H). |
| 242 | N-(1′-(2-(1,1-difluoroethyl)-6-(6-methylpyridin-3-yl)pyrimidin-4-yl)-1′,2′- dihydrospiro[cyclopropane-1,3′-pyrrolo[3,2-c]pyridin]-6′-yl)acetamide 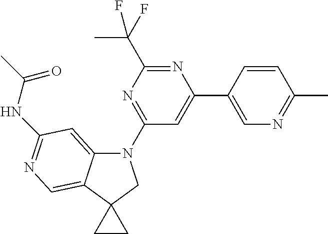 [SMILES: CC(=O)Nc1cc2c(cn1)C1(CC1)CN2c1cc(-c2ccc(C)nc2)nc(C(C)(F)F)n1] Boronate: 2-methyl-5-(4,4,5,5-tetramethyl-1,3,2-dioxaborolan-2-yl)pyridine 5.6 mg, 12.2% yield. LCMS m/z = 437.3 [M + H]⁺ |
| 243^{A} | N-(1′-(2-(1,1-difluoroethyl)-6-(6-methoxypyridin-3-yl)pyrimidin-4-yl)-1′,2′- dihydrospiro[cyclopropane-1,3′-pyrrolo[3,2-c]pyridin]-6′-yl)acetamide trifluoroacetate 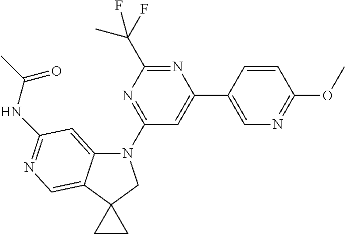 [SMILES: COc1ccc(-c2cc(N3CC4(CC4)c4cnc(NC(C)=O)cc43)nc(C(C)(F)F)n2)cn1] Boronate: 2-methoxy-5-(4,4,5,5-tetramethyl-1,3,2-dioxaborolan-2- yl)pyridine 7.0 mg, 14.7% yield. LCMS m/z = 453.3 [M + H]⁺ ¹H NMR (DMSO-d₆) δ: 11.27-10.26 (m, 1H), 9.09 (s, 1H), 8.94-8.70 (m, 1H), 8.59-8.42 (m, 1H), 7.74 (s, 1H), 7.47 (s, 1H), 7.02 (d, J = 8.8 Hz, 1H), 4.38 (s, 2H), 3.96 (s, 3H), 2.22 (s, 3H), 2.15 (s, 3H), 1.27 (br s, 2H), 1.25- 1.18 (m, 2H) |

A the compound was purified by HPLC (Waters Sunfire OBD C18 PREP 30×100 mm, 5 um column with mobile phase 95% H₂O/5% MeCN to 35% H₂O/65% MeCN over 8 min, (Trifluoroacetic acid 0.1% (v/v) modifier) with flow rate at 50 mL/min.).

### Examples 244 to 255

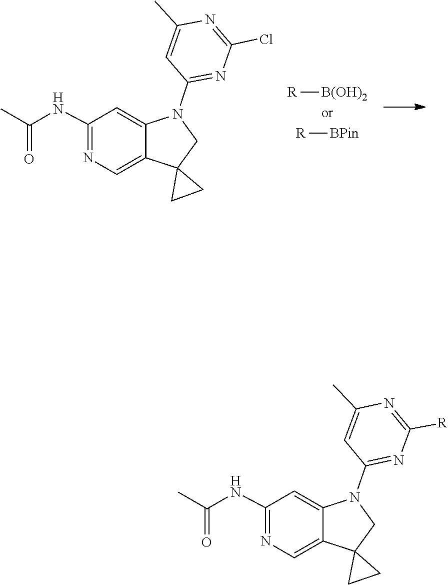

An 8 mL vial was charged with N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 16.5 mg, 50 μmol), R-BPin (100 μmol), Pd(dppf)Cl₂·DCM (4.1 mg, 5 μmol) and Cs₂CO₃ (48.9 mg, 150 μmol). Dioxane/water (4:1, 1.0 mL, 0.2 M) was added under inert atmosphere. The vial was sealed with a Teflon cap, and the reaction solution was heated under reflux at 90° C. for 3 h. The cooled reaction mixture was diluted with EtOAc, washed with H₂O (3 mL), and extracted with EtOAc (5 mL, 2×). The combined organic phases were dried over anhydrous MgSO₄ and concentrated in vacuo to give the crude product. This was purified by HPLC using a Waters SunFire Prep C18 column (5 um OBD 19×100 mm) under acidic conditions, or a Waters XSelect CSH Prep C18 column (5 um OBD 19×100 mm) under basic conditions.
Basic conditions (Xselect OBD 19): MeCN, water, 0.2% NH₄H modifier
Acidic conditions (Sunfire OBD 19): MeCN, water, 0.1% TFA modifier

| Example No | Name, Structure, R-BPin, HPLC, Data |
|---|---|
| 244 | N-(1′-(6-methyl-2-(1-methyl-1H-pyrazol-3-yl)pyrimidin-4-yl)-1′,2′- dihydrospiro[cyclopropane-1,3′-pyrrolo[3,2-c]pyridin]-6′-yl)acetamide trifluoroacetate 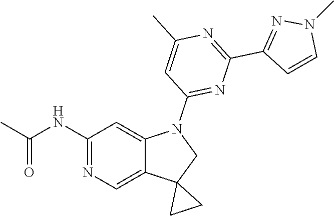 [SMILES: CC(=O)Nc1cc2c(cn1)C1(CC1)CN2c1cc(C)nc(-c2ccn(C)n2)n1] R-BPin: 1-methyl-3-(4,4,5,5-tetramethyl-1,3,2-dioxaborolan-2-yl)-1H- pyrazole HPLC: Acidic conditions (Sunfire OBD 19): Gradient 5-45% 9.10 mg, LCMS m/z = 376.2 [M + H]⁺ |
| 245 | N-(1′-(6-methyl-2-(1-methyl-1H-pyrazol-4-yl)pyrimidin-4-yl)-1′,2′- dihydrospiro[cyclopropane-1,3′-pyrrolo[3,2-c]pyridin]-6′-yl)acetamide trifluoroacetate 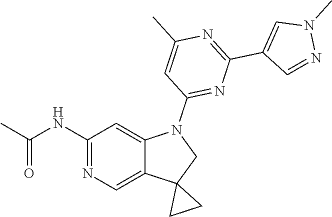 [SMILES: CC(=O)Nc1cc2c(cn1)C1(CC1)CN2c1cc(C)nc(-c2cnn(C)c2)n1] R-BPin: 1-methyl-4-(4,4,5,5-tetramethyl-1,3,2-dioxaborolan-2-yl)-1H- pyrazole HPLC: Acidic conditions (Sunfire OBD 19): Gradient 5-50% 2.50 mg, LCMS m/z = 376.2 [M + H]⁺ |
| 246 | N-(1′-(2-(1-cyclobutyl-1H-pyrazol-4-yl)-6-methylpyrimidin-4-yl)-1′,2′- dihydrospiro[cyclopropane-1,3′-pyrrolo[3,2-c]pyridin]-6′-yl)acetamide -N 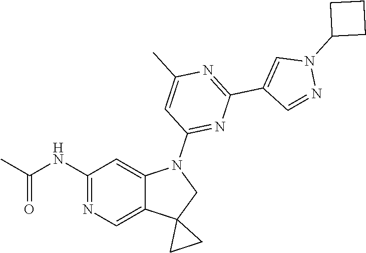 [SMILES: CC(=O)Nc1cc2c(cn1)C1(CC1)CN2c1cc(C)nc(-c2cnn(C3CCC3)c2)n1] R-BPin: 1-cyclobutyl-4-(4,4,5,5-tetramethyl-1,3,2-dioxaborolan-2-yl)- 1H-pyrazole HPLC: Basic conditions (Xselect OBD 19): Gradient 5-60% 0.8 mg, LCMS m/z = 416.3 [M + H]⁺ |
| 247 | N-(1′-(2-(1-ethyl-1H-pyrazol-4-yl)-6-methylpyrimidin-4-yl)-1′,2′- dihydrospiro[cyclopropane-1,3′-pyrrolo[3,2-c]pyridin]-6′-yl)acetamide 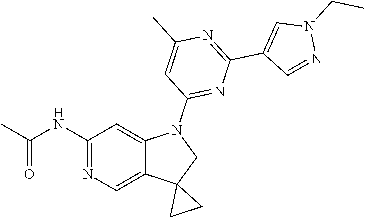 [SMILES: CCn1cc(-c2nc(C)cc(N3CC4(CC4)c4cnc(NC(C)=O)cc43)n2)cn1] R-BPin: 1-ethyl-4-(4,4,5,5-tetramethyl-1,3,2-dioxaborolan-2-yl)-1H- pyrazole HPLC: Basic conditions (Xselect OBD 19): Gradient 5-55% 4.4 mg, LCMS m/z = 390.3 [M + H]⁺ |
| 248 | N-(1′-(2-(5-(difluoromethyl)-1-methyl-1H-pyrazol-4-yl)-6- methylpyrimidin-4-yl)-1′,2′-dihydrospiro[cyclopropane-1,3′-pyrrolo[3,2- c]pyridin]-6′-yl)acetamide 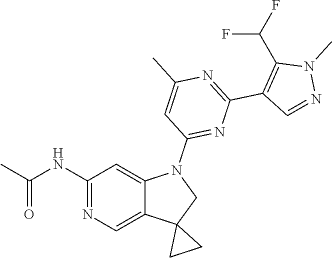 [SMILES: CC(=O)Nc1cc2c(cn1)C1(CC1)CN2c1cc(C)nc(-c2cnn(C)c2C(F)F)n1] R-BPin: 5-(difluoromethyl)-1-methyl-4-(4,4,5,5-tetramethyl-1,3,2- dioxaborolan-2-yl)-1H-pyrazole HPLC: Basic conditions (Xselect OBD 19): Gradient 5-65% 3.7 mg, LCMS m/z = 426.2 [M + H]⁺ |
| 249 | N-(1′-(2-(1-isopropyl-1H-pyrazol-4-yl)-6-methylpyrimidin-4-yl)-1′,2′- dihydrospiro[cyclopropane-1,3′-pyrrolo[3,2-c]pyridin]-6′-yl)acetamide 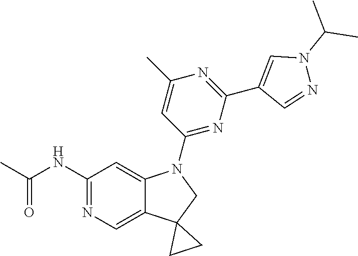 [SMILES: CC(=O)Nc1cc2c(cn1)C1(CC1)CN2c1cc(C)nc(-c2cnn(C(C)C)c2)n1] R-BPin: 1-isopropyl-4-(4,4,5,5-tetramethyl-1,3,2-dioxaborolan-2-yl)-1H- pyrazole HPLC: Basic conditions (Xselect OBD 19): Gradient 5-60% 1.6 mg, LCMS m/z = 404.3 [M + H]⁺ |
| 250 | N-(1′-(2-(1-cyclopropyl-1H-pyrazol-4-yl)-6-methylpyrimidin-4-yl)-1′,2′- dihydrospiro[cyclopropane-1,3′-pyrrolo[3,2-c]pyridin]-6′-yl)acetamide 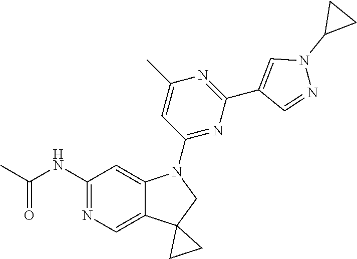 [SMILES: CC(=O)Nc1cc2c(cn1)C1(CC1)CN2c1cc(C)nc(-c2cnn(C3CC3)c2)n1] R-BPin: 1-cyclopropyl-4-(4,4,5,5-tetramethyl-1,3,2-dioxaborolan-2-yl)- 1H-pyrazole HPLC: Basic conditions (Xselect OBD 19): Gradient 5-55% 3.9 mg, LCMS m/z = 402.3 [M + H]⁺ |
| 251 | N-(1′-(6-methyl-2-(1-(oxetan-3-yl)-1H-pyrazol-4-yl)pyrimidin-4-yl)- 1′,2′-dihydrospiro[cyclopropane-1,3′-pyrrolo[3,2-c]pyridin]-6′- yl)acetamide trifluoroacetate 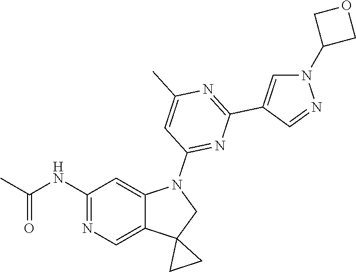 [SMILES: CC(=O)Nc1cc2c(cn1)C1(CC1)CN2c1cc(C)nc(-c2cnn(C3COC3)c2)n1] R-BPin: 1-(oxetan-3-yl)-4-(4,4,5,5-tetramethyl-1,3,2-dioxaborolan-2-yl)- 1H-pyrazole HPLC: Acidic conditions (Sunfire OBD 19): Gradient 5-50% 3.1 mg, LCMS m/z = 418.2 [M + H]⁺ |
| 252 | N-(1′-(6-methyl-2-(1H-pyrazol-3-yl)pyrimidin-4-yl)-1′,2′- dihydrospiro[cyclopropane-1,3′-pyrrolo[3,2-c]pyridin]-6′-yl)acetamide trifluoroacetate 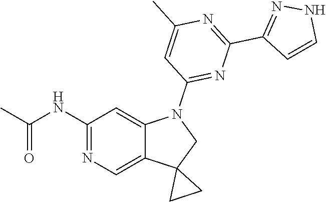 [SMILES: CC(=O)Nc1cc2c(cn1)C1(CC1)CN2c1cc(C)nc(-c2cc[nH]n2)n1] R-BPin: 4-(4,4,5,5-tetramethyl-1,3,2-dioxaborolan-2-yl)-1H-pyrazole HPLC: Acidic conditions (Sunfire OBD 19): Gradient 5-50% 1.3 mg, LCMS m/z = 362.2 [M + H]⁺ |
| 253 | N-(1′-(2-(1-(cyclopropylmethyl)-1H-pyrazol-4-yl)-6-methylpyrimidin-4- yl)-1′,2′-dihydrospiro[cyclopropane-1,3′-pyrrolo[3,2-c]pyridin]-6′- yl)acetamide trifluoroacetate 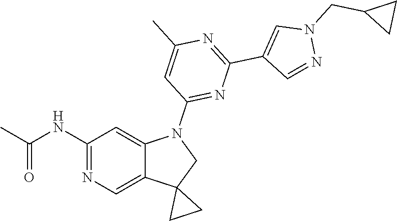 [SMILES: CC(=O)Nc1cc2c(cn1)C1(CC1)CN2c1cc(C)nc(-c2cnn(CC3CC3)c2)n1] R-BPin: 1-cyclopropylmethyl-4-(4,4,5,5-tetramethyl-1,3,2-dioxaborolan- 2-yl)-1H-pyrazole HPLC: Acidic conditions (Sunfire OBD 19): Gradient 5-60% 5.5 mg, LCMS m/z = 416.3 [M + H]⁺ |
| 254* | N-(1′-(2-(6-methoxypyridin-3-yl)-6-methylpyrimidin-4-yl)-1′,2′- dihydrospiro[cyclopropane-1,3′-pyrrolo[3,2-c]pyridin]-6′-yl)acetamide trifluoroacetate 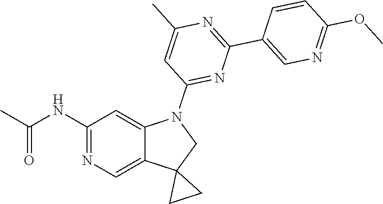 [SMILES: COc1ccc(-c2nc(C)cc(N3CC4(CC4)c4cnc(NC(C)=O)cc43)n2)cn1] R-B(OH)2: (6-methoxypyridin-3-yl)boronic acid HPLC: Acidic conditions (Sunfire OBD 19): Gradient 5-65% 4.2 mg, LCMS m/z = 403.2 [M + H]⁺ |
| 255 | N-(1′-(6-methyl-2-(2-methylthiazol-5-yl)pyrimidin-4-yl)-1′,2′- dihydrospiro[cyclopropane-1,3′-pyrrolo[3,2-c]pyridin]-6′-yl)acetamide trifluoroacetate 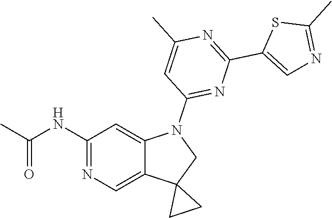 [SMILES: CC(=O)Nc1cc2c(cn1)C1(CC1)CN2c1cc(C)nc(-c2cnc(C)s2)n1] R-BPin: 2-methyl-5-(4,4,5,5-tetramethyl-1,3,2-dioxaborolan-2- yl)thiazole HPLC: Acidic conditions (Sunfire OBD 19): Gradient 5-60% 0.5 mg, LCMS m/z = 393.1 [M + H]⁺ |

*R-B(OH)₂ was used instead of R-BPin

### Examples 256 to 267

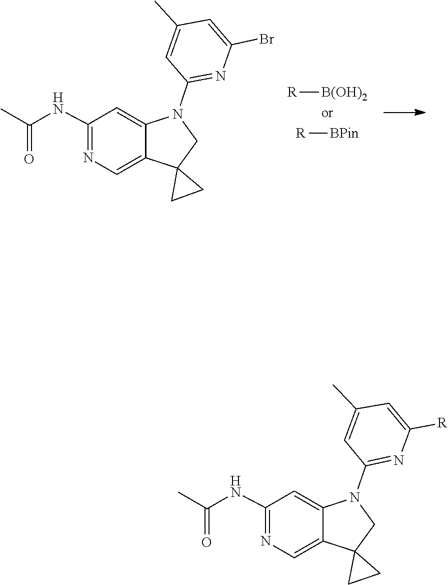

An 8 mL vial was charged with N-(1′-(6-bromo-4-methylpyridin-2-yl)-1′,2′-dihydrospiro[cyclopropane-1,3′-pyrrolo[3,2-c]pyridin]-6′-yl)acetamide (Preparation 157, 18.7 mg, 50 μmol), RBPin (100 μmol), Pd(dppf)Cl₂·DCM (4.1 mg, 5 μmol) and Cs₂CO₃ (48.9 mg, 150 μmol). Dioxane/water (4:1, 1.0 mL, 0.2 M) was added under inert atmosphere. The vial was sealed with a Teflon cap, and the reaction was heated under reflux at 90° C. for 3 h. The cooled reaction mixture was diluted with EtOAc, washed with H₂O (3 mL), and extracted with EtOAc (5 mL, 2×). The combined organic phases were dried over anhydrous MgSO₄ and concentrated in vacuo to give the crude product. This was purified by HPLC using a Waters SunFire Prep C18 column (5 um OBD 30×100 mm) under acidic conditions, (Sunfire OBD 30): MeCN, water, 0.1% TFA modifier

| Example No | Name, Structure, R-B(OH)₂ or R-BPIn, HPLC, Data |
|---|---|
| 256 | N-(1′-(4-methyl-6-(1-methyl-1H-pyrazol-3-yl)pyridin-2-yl)-1′,2′- dihydrospiro[cyclopropane-1,3′-pyrrolo[3,2-c]pyridin]-6′-yl)acetamide trifluoroacetate 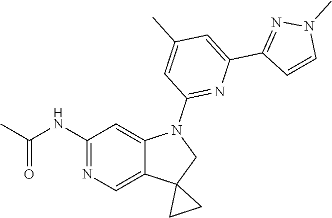 [SMILES: CC(=O)Nc1cc2c(cn1)C1(CC1)CN2c1cc(C)cc(-c2ccn(C)n2)n1] R-BPin: 1-methyl-3-(4,4,5,5-tetramethyl-1,3,2-dioxaborolan-2-yl)-1H- pyrazole HPLC: Acidic conditions (Sunfire OBD 30): Gradient 5-60% 6.7 mg, LCMS m/z = 375.3 [M + H]⁺ |
| 257 | N-(1′-(4-methyl-6-(1-methyl-1H-pyrazol-4-yl)pyridin-2-yl)-1′,2′- dihydrospiro[cyclopropane-1,3′-pyrrolo[3,2-c]pyridin]-6′-yl)acetamide trifluoroacetate 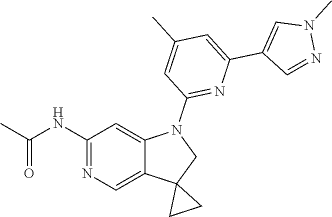 [SMILES: CC(=O)Nc1cc2c(cn1)C1(CC1)CN2c1cc(C)cc(-c2cnn(C)c2)n1] R-BPin: 1-methyl-4-(4,4,5,5-tetramethyl-1,3,2-dioxaborolan-2-yl)-1H- pyrazole HPLC: Acidic conditions (Sunfire OBD 30): Gradient 5-60% 3.0 mg, LCMS m/z = 375.3 [M + H]⁺ |
| 258 | N-(1′-(4-methyl-6-(1-(methylsulfonyl)-1H-pyrazol-4-yl)pyridin-2-yl)-1′,2′- dihydrospiro[cyclopropane-1,3′-pyrrolo[3,2-c]pyridin]-6′-yl)acetamide trifluoroacetate 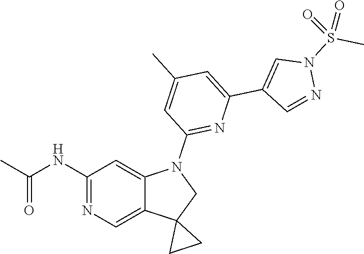 [SMILES: CC(=O)Nc1cc2c(cn1)C1(CC1)CN2c1cc(C)cc(-c2cnn(S(C)(=O)=O)c2)n1] R-BPin: 1-(methylsulfonyl)-4-(4,4,5,5-tetramethyl-1,3,2-dioxaborolan-2- yl)-1H-pyrazole HPLC: Acidic conditions (Sunfire OBD 30): Gradient 5-65% 3.6 mg, LCMS m/z = 439.2 [M + H]⁺ |
| 259 | N-(1′-(6-(1-(difluoromethyl)-1H-pyrazol-4-yl)-4-methylpyridin-2-yl)-1′,2′- dihydrospiro[cyclopropane-1,3′-pyrrolo[3,2-c]pyridin]-6′-yl)acetamide trifluoroacetate 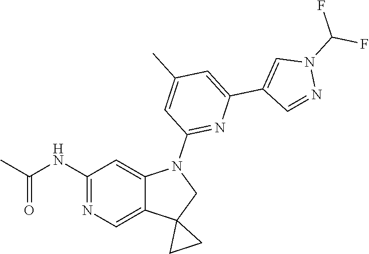 [SMILES: CC(=O)Nc1cc2c(cn1)C1(CC1)CN2c1cc(C)cc(-c2cnn(C(F)F)c2)n1] R-BPin: 1-(difluoromethyl)-4-(4,4,5,5-tetramethyl-1,3,2-dioxaborolan-2- yl)-1H-pyrazole HPLC: Acidic conditions (Sunfire OBD 30): Gradient 5-70% 6.7 mg, LCMS m/z = 411.3 [M + H]⁺ |
| 260 | N-(1′-(6-(1-ethyl-1H-pyrazol-4-yl)-4-methylpyridin-2-yl)-1′,2′- dihydrospiro[cyclopropane-1,3′-pyrrolo[3,2-c]pyridin]-6′-yl)acetamide trifluoroacetate 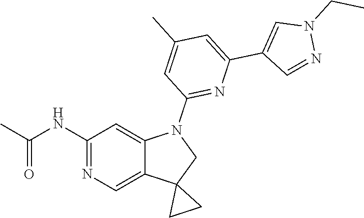 [SMILES: CCn1cc(-c2cc(C)cc(N3CC4(CC4)c4cnc(NC(C)=O)cc43)n2)cn1] R-BPin: 1-ethyl-4-(4,4,5,5-tetramethyl-1,3,2-dioxaborolan-2-yl)-1H- pyrazole HPLC: Acidic conditions (Sunfire OBD 30): Gradient 5-65% 10.2 mg, LCMS m/z = 389.3 [M + H]⁺ |
| 261* | N-(1′-(4-methyl-6-(1-methyl-3-(trifluoromethyl)-1H-pyrazol-4-yl)pyridin- 2-yl)-1′,2′-dihydrospiro[cyclopropane-1,3′-pyrrolo[3,2-c]pyridin]-6′- yl)acetamide trifluoroacetate 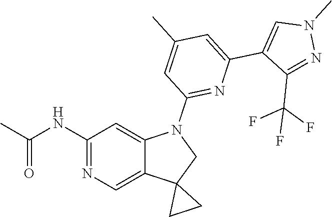 [SMILES: CC(=O)Nc1cc2c(cn1)C1(CC1)CN2c1cc(C)cc(-c2cn(C)nc2C(F)(F)F)n1] R-B(OH)₂: (1-methyl-3-(trifluoromethyl)-1H-pyrazol-4-yl)boronic acid HPLC: Acidic conditions (Sunfire OBD 30): Gradient 20-75% 1.3 mg, LCMS m/z = 443.2 [M + H]⁺ |
| 262 | N-(1′-(6-(5-(difluoromethyl)-1-methyl-1H-pyrazol-4-yl)-4-methylpyridin- 2-yl)-1′,2′-dihydrospiro[cyclopropane-1,3′-pyrrolo[3,2-c]pyridin]-6′- yl)acetamide trifluoroacetate 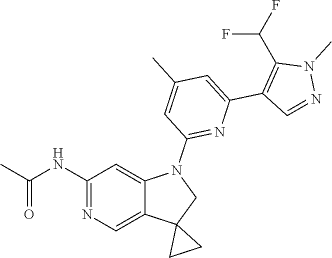 [SMILES: CC(=O)Nc1cc2c(cn1)C1(CC1)CN2c1cc(C)cc(-c2cnn(C)c2C(F)F)n1] R-BPin: 5-(difluoromethyl)-1-methyl-4-(4,4,5,5-tetramethyl-1,3,2- dioxaborolan-2-yl)-1H-pyrazole HPLC: Acidic conditions (Sunfire OBD 30): Gradient 5-65% 8.5 mg, LCMS m/z = 425.3 [M + H]⁺ |
| 263 | N-(1′-(6-(1-isopropyl-1H-pyrazol-4-yl)-4-methylpyridin-2-yl)-1′,2′- dihydrospiro[cyclopropane-1,3′-pyrrolo[3,2-c]pyridin]-6′-yl)acetamide trifluoroacetate 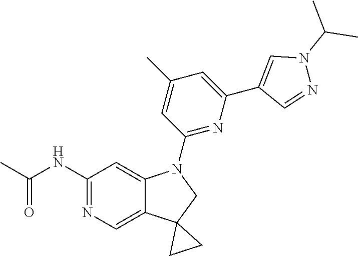 [SMILES: CC(=O)Nc1cc2c(cn1)C1(CC1)CN2c1cc(C)cc(-c2cnn(C(C)C)c2)n1] R-BPin: 1-isopropyl-4-(4,4,5,5-tetramethyl-1,3,2-dioxaborolan-2-yl)-1H- pyrazole HPLC: Acidic conditions (Sunfire OBD 30): Gradient 5-70% 6.0 mg, LCMS m/z = 403.3 [M + H]⁺ |
| 264 | N-(1′-(6-(1-cyclopropyl-1H-pyrazol-4-yl)-4-methylpyridin-2-yl)-1′,2′- dihydrospiro[cyclopropane-1,3′-pyrrolo[3,2-c]pyridin]-6′-yl)acetamide trifluoroacetate 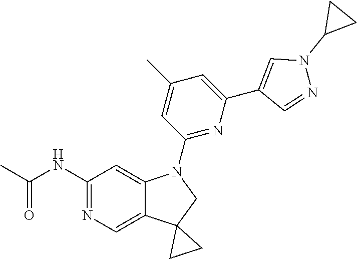 [SMILES: CC(=O)Nc1cc2c(cn1)C1(CC1)CN2c1cc(C)cc(-c2cnn(C3CC3)c2)n1] R-BPin: 1-cyclopropyl-4-(4,4,5,5-tetramethyl-1,3,2-dioxaborolan-2-yl)- 1H-pyrazole HPLC: Acidic conditions (Sunfire OBD 30): Gradient 5-65% 2.7 mg, LCMS m/z = 401.3 [M + H]⁺ |
| 265 | N-(1′-(4-methyl-6-(1-(oxetan-3-yl)-1H-pyrazol-4-yl)pyridin-2-yl)-1′,2′- dihydrospiro[cyclopropane-1,3′-pyrrolo[3,2-c]pyridin]-6′-yl)acetamide trifluoroacetate 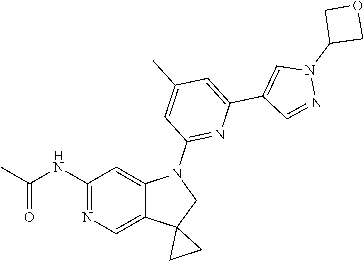 [SMILES: CC(=O)Nc1cc2c(cn1)C1(CC1)CN2c1cc(C)cc(-c2cnn(C3COC3)c2)n1] R-BPin: 1-(oxetan-3-yl)-4-(4,4,5,5-tetramethyl-1,3,2-dioxaborolan-2-yl)- 1H-pyrazole HPLC: Acidic conditions (Sunfire OBD 30): Gradient 5-60% 5.8 mg, LCMS m/z = 417.3 [M + H]⁺ |
| 266 | N-(1′-(6-(1-(cyclopropylmethyl)-1H-pyrazol-4-yl)-4-methylpyridin-2-yl)- 1′,2′-dihydrospiro[cyclopropane-1,3′-pyrrolo[3,2-c]pyridin]-6′- yl)acetamide trifluoroacetate 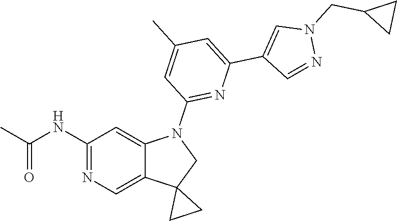 [SMILES: CC(=O)Nc1cc2c(cn1)C1(CC1)CN2c1cc(C)cc(-c2cnn(CC3CC3)c2)n1] R-BPin: 1-(cyclopropylmethyl)-4-(4,4,5,5-tetramethyl-1,3,2-dioxaborolan- 2-yl)-1H-pyrazole HPLC: Acidic conditions (Sunfire OBD 30): Gradient 5-70% 3.5 mg, LCMS m/z = 415.3 [M + H]⁺ Rf Analytical (Amm/TFA): 2.21/1.75 |
| 267* | N-(1′-(6′-methoxy-4-methyl-[2,3′-bipyridin]-6-yl)-1′,2′- dihydrospiro[cyclopropane-1,3′-pyrrolo[3,2-c]pyridin]-6′-yl)acetamide trifluoroacetate 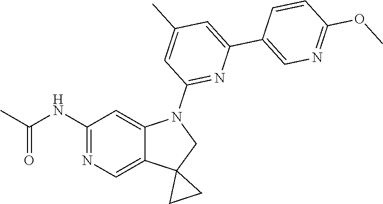 [SMILES: COc1ccc(-c2cc(C)cc(N3CC4(CC4)c4cnc(NC(C)=O)cc43)n2)cn1] R-B(OH)₂: (6-methoxypyridin-3-yl)boronic acid HPLC: Acidic conditions (Sunfire OBD 30): Gradient 5-70% 3.8 mg, LCMS m/z = 402.3 [M + H]⁺ |

*R-B(OH)₂ was used instead of R-BPin ester

### Example 268

### N-(1′-(6-(1-(azetidin-3-yl)-1H-pyrazol-4-yl)-4-methylpyridin-2-yl)-1′,2′-dihydrospiro[cyclopropane-1,3′-pyrrolo[3,2-c]pyridin]-6′-yl)acetamide trifluoroacetate

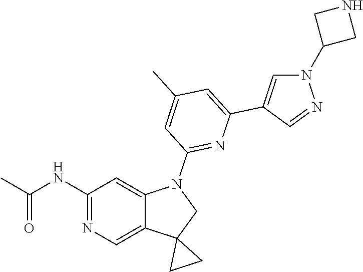 [SMILES: CC(=O)Nc1cc2c(cn1)C1(CC1)CN2c1cc(C)cc(-c2cnn(C3CNC3)c2)n1]

An 8 mL vial was charged with N-(1′-(6-bromo-4-methylpyridin-2-yl)-1′,2′-dihydrospiro[cyclopropane-1,3′-pyrrolo[3,2-c]pyridin]-6′-yl)acetamide (Preparation 157, 18.7 mg, 50 μmol), tert-butyl 3-(4-(4,4,5,5-tetramethyl-1,3,2-dioxaborolan-2-yl)-1H-pyrazol-1-yl)azetidine-1-carboxylate (34.9 mg, 100 μmol), Pd(dppf)Cl₂·DCM (4.1 mg, 5 μmol) and Cs₂CO₃ (48.9 mg, 150 μmol). Dioxane/water (4:1, 1.0 mL, 0.2 M) was added under an inert atmosphere. The vial was sealed with a Teflon cap, and the reaction was heated under reflux at 90° C. for 3 h. The cooled reaction mixture was diluted with EtOAc, washed with H₂O (3 mL), and extracted with EtOAc (5 mL, 2×). The combined organic phases were dried over anhydrous MgSO₄ and concentrated in vacuo. The crude material was dissolved in DCM (1 mL, 0.2 M) and TFA (7.7 μL, 100 μmol) was added to the reaction solution. The reaction mixture was stirred at rt for 16 h. The reaction mixture was diluted with DCM (3 mL) and washed with sat. aq. NaHCO₃. The layers were separated, and the aqueous phase was extracted with DCM (5 mL, 2×). The combined organic phases were dried over Na₂SO₄ and concentrated in vacuo to give the crude product and purified by Waters SunFire Prep C18 column (5 um OBD 30×100 mm, purification gradient: 5-65%, purification modifier: 0.1% TFA.) 11.2 mg, LCMS m/z=416.3 [M+H]⁺

### Example 269

### N-(1′-(2-(1-(azetidin-3-yl)-1H-pyrazol-4-yl)-6-methylpyrimidin-4-yl)-1′,2′-dihydrospiro[cyclopropane-1,3′-pyrrolo[3,2-c]pyridin]-6′-yl)acetamide trifluoroacetate

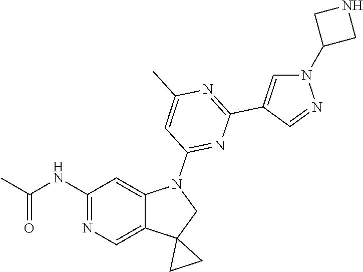 [SMILES: CC(=O)Nc1cc2c(cn1)C1(CC1)CN2c1cc(C)nc(-c2cnn(C3CNC3)c2)n1]

N-(1′-(2-(1-(Azetidin-3-yl)-1H-pyrazol-4-yl)-6-methylpyrimidin-4-yl)-1′,2′-dihydrospiro[cyclopropane-1,3′-pyrrolo[3,2-c]pyridin]-6′-yl)acetamide trifluoroacetate was obtained, 2.4 mg, from N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153) and tert-butyl 3-(4-(4,4,5,5-tetramethyl-1,3,2-dioxaborolan-2-yl)-1H-pyrazol-1-yl)azetidine-1-carboxylate following the procedure described in Example 268. LCMS m/z=417.3 [M+H]⁺

### Example 270

### N-(1′-(4-(1,1-difluoroethyl)pyrimidin-2-yl)-1′,2′-dihydrospiro[piperidine-4,3′-pyrrolo[3,2-c]pyridin]-6′-yl)acetamide

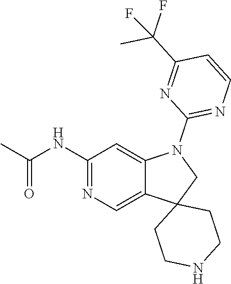 [SMILES: CC(=O)Nc1cc2c(cn1)C1(CCNCC1)CN2c1nccc(C(C)(F)F)n1]

To a solution of tert-butyl 6′-acetamido-1′-(4-(1,1-difluoroethyl)pyrimidin-2-yl)-1′,2′-dihydrospiro[piperidine-4,3′-pyrrolo[3,2-c]pyridine]-1-carboxylate (Preparation 150, 100 mg, 205 μmol) in DCM (2 mL) was added TFA (298 mg, 2.6 mmol). The reaction mixture was stirred at rt overnight, then concentrated under vacuum. The residue was diluted with EtOAc and washed with aq. NaHCO₃. The organic layer was separated, dried and concentrated under reduced pressure to give N-(1′-(4-(1,1-difluoroethyl)pyrimidin-2-yl)-1′,2′-dihydrospiro[piperidine-4,3′-pyrrolo[3,2-c]pyridin]-6′-yl)acetamide (26 mg, 33% yield) as a colorless oil. LCMS m/z=389.2 [M+H]⁺. ¹H NMR: (400 MHz, MeOH-d₄) δ: ppm 8.98 (s, 1H), 8.80 (d, 1H, J=5.0 Hz), 8.05 (s, 1H), 7.25 (d, 1H, J=5.0 Hz), 3.30-3.20 (m, 2H), 2.97 (dt, 2H, J=2.6, 12.9 Hz), 2.20-2.10 (m, 6H), 2.10-2.00 (m, 2H), 1.90-1.80 (m, 2H).

### Example 271

### N-(1′-(2-(1,1-difluoroethyl)pyrimidin-4-yl)-1′,2′-dihydrospiro[piperidine-4,3′-pyrrolo[3,2-c]pyridin]-6′-yl)acetamide

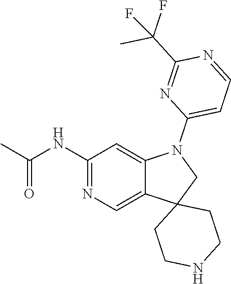 [SMILES: CC(=O)Nc1cc2c(cn1)C1(CCNCC1)CN2c1ccnc(C(C)(F)F)n1]

N-(1′-(2-(1,1-Difluoroethyl)pyrimidin-4-yl)-1′,2′-dihydrospiro[piperidine-4,3′-pyrrolo[3,2-c]pyridin]-6′-yl)acetamide was prepared, 3 mg, using a similar method to the one described in Example 270 from tert-butyl 6′-acetamido-1′-(2-(1,1-difluoroethyl)pyrimidin-4-yl)-1′,2′-dihydrospiro[piperidine-4,3′-pyrrolo[3,2-c]pyridine]-1-carboxylate (Preparation 151). LCMS m/z=389.2 [M+H]⁺. ¹H NMR: (400 MHz, MeOH-d₄) δ: ppm 9.07 (s, 1H), 8.62 (d, 1H, J=6.0 Hz), 8.11 (s, 1H), 7.10-7.00 (m, 1H), 4.22 (s, 2H), 3.51-3.48 (m, 2H), 3.30-3.20 (m, 2H), 2.20-2.10 (m, 8H), 2.10-2.00 (m, 2H).

### Example 272

### N-(1′-(6-(1,1-difluoroethyl)pyridin-2-yl)-1′,2′-dihydrospiro[piperidine-4,3′-pyrrolo[3,2-c]pyridin]-6′-yl)acetamide

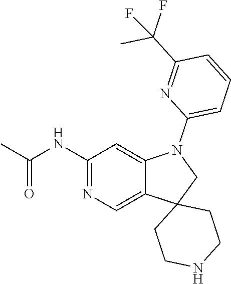 [SMILES: CC(=O)Nc1cc2c(cn1)C1(CCNCC1)CN2c1cccc(C(C)(F)F)n1]

N-(1′-(6-(1,1-Difluoroethyl)pyridin-2-yl)-1′,2′-dihydrospiro[piperidine-4,3′-pyrrolo[3,2-c]pyridin]-6′-yl)acetamide was obtained, 28 mg, using a similar method to the one described in Example 270 from tert-butyl 6′-acetamido-1′-(6-(1,1-difluoroethyl)pyridin-2-yl)-1′,2′-dihydrospiro[piperidine-4,3′-pyrrolo[3,2-c]pyridine]-1-carboxylate (Preparation 152). LCMS m/z=388.2 [M+H]⁺. ¹H NMR: (400 MHz, MeOH-d₄) δ: ppm 8.90-8.80 (m, 1H), 7.99 (s, 1H), 7.92 (t, 1H, J=8.0 Hz), 7.32 (d, 1H, J=7.3 Hz), 7.12 (d, 1H, J=8.3 Hz), 4.19 (s, 2H), 3.50-3.40 (m, 2H), 3.30-3.20 (m, 2H), 2.20-2.00 (m, 9H).

### Example 273

### N-(1′-(6-amino-2-(1,1-difluoroethyl)pyrimidin-4-yl)-1′,2′-dihydrospiro[cyclopropane-1,3′-pyrrolo[3,2-c]pyridin]-6′-yl)acetamide trifluoroacetate

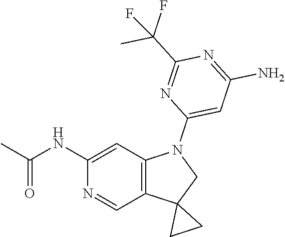 [SMILES: CC(=O)Nc1cc2c(cn1)C1(CC1)CN2c1cc(N)nc(C(C)(F)F)n1]

To a vial was added N-(1′-(2-(1,1-difluoroethyl)-6-((2,4-dimethoxybenzyl)amino)pyrimidin-4-yl)-1′,2′-dihydrospiro[cyclopropane-1,3′-pyrrolo[3,2-c]pyridin]-6′-yl)acetamide (Preparation 154, 202 mg, 395.66 μmol), DCM (1.5 mL) and TFA (0.5 mL) and the reaction mixture was stirred at rt overnight. The solvent was removed in vacuo and the crude mixture was purified by HPLC Column: Sunfire Prep C18 OBD 5 um 30×50 mm, Gradient: 5-40% MeCN in water, modifier 0.2% TFA at 50 mL/min to give N-(1′-(6-amino-2-(1,1-difluoroethyl)pyrimidin-4-yl)-1′,2′-dihydrospiro[cyclopropane-1,3′-pyrrolo[3,2-c]pyridin]-6′-yl)acetamide, 4.6 mg. LCMS m/z=361 [M+H]⁺

### Example 274

### N-(1′-(4-(1,1-difluoroethyl)pyrimidin-2-yl)-1-methyl-1′,2′-dihydrospiro[piperidine-4,3′-pyrrolo[3,2-c]pyridin]-6′-yl)acetamide trifluorocetate

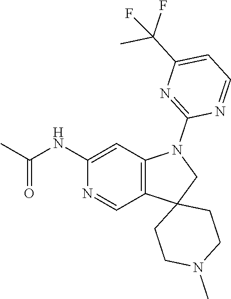 [SMILES: CC(=O)Nc1cc2c(cn1)C1(CCN(C)CC1)CN2c1nccc(C(C)(F)F)n1]

To a mixture of N-(1′-(4-(1,1-difluoroethyl)pyrimidin-2-yl)-1′,2′-dihydrospiro[piperidine-4,3′-pyrrolo[3,2-c]pyridin]-6′-yl)acetamide (Preparation 268, 20 mg, 51 μmol), formaldehyde (12.7 M, 81 μL) and acetic acid (3.09 mg, 51 μmol) in DCM (0.5 mL) was added NaBH(OAc)₃ (22 mg, 103 μmol). The reaction mixture was stirred at rt overnight. The mixture was diluted with water, the organic layer was separated, dried and concentrated under reduced pressure. The crude was purified by HPLC (Waters XSelect CSH Prep C18 5 um OBD 30×100 mm; MeCN in water, modifier 0.1% TFA) to give N-(1′-(4-(1,1-difluoroethyl)pyrimidin-2-yl)-1-methyl-1′,2′-dihydrospiro[piperidine-4,3′-pyrrolo[3,2-c]pyridin]-6′-yl)acetamide (25 mg, 65% yield, TFA salt) as a white solid. LCMS m/z=403.3 [M+H]⁺. ¹H NMR: (400 MHz, MeOH-d₄) δ: ppm 8.95 (d, 1H, J=5.0 Hz), 8.33 (s, 1H), 8.06 (s, 1H), 7.50-7.49 (m, 1H), 4.56 (s, 2H), 3.63 (br d, 2H, J=13.3 Hz), 3.40-3.30 (m, 2H), 2.98 (s, 3H), 2.40-2.20 (m, 7H), 2.08 (t, 3H, J=18.8 Hz).

### Example 275

### N-(1′-(2-(1,1-difluoroethyl)pyrimidin-4-yl)-1-methyl-1′,2′-dihydrospiro[piperidine-4,3′-pyrrolo[3,2-c]pyridin]-6′-yl)acetamide trifluoroacetate

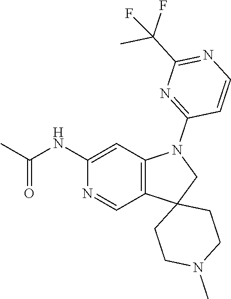 [SMILES: CC(=O)Nc1cc2c(cn1)C1(CCN(C)CC1)CN2c1ccnc(C(C)(F)F)n1]

N-(1′-(2-(1,1-difluoroethyl)pyrimidin-4-yl)-1-methyl-1′,2′-dihydrospiro[piperidine-4,3′-pyrrolo[3,2-c]pyridin]-6′-yl)acetamide (7 mg) was prepared using a similar method to the one described in Example 274 from N-(1′-(2-(1,1-difluoroethyl)pyrimidin-4-yl)-1′,2′-dihydrospiro[piperidine-4,3′-pyrrolo[3,2-c]pyridin]-6′-yl)acetamide (Example 271). LCMS m/z=403.3 [M+H]⁺. ¹H NMR: (400 MHz, MeOH-d₄) δ: ppm 8.82 (d, 1H, J=6.0 Hz), 8.51 (s, 1H), 8.10 (s, 1H), 7.23 (d, 1H, J=5.8 Hz), 4.38 (s, 2H), 3.70-3.60 (m, 2H), 3.30-3.20 (m, 2H), 2.99 (s, 3H), 2.40-2.30 (m, 5H), 2.30-2.20 (m, 2H), 2.12 (t, 3H, J=18.7 Hz).

### Example 276

### N-(1′-(6-(1,1-difluoroethyl)pyridin-2-yl)-1-methyl-1′,2′-dihydrospiro[piperidine-4,3′-pyrrolo[3,2-c]pyridin]-6′-yl)acetamide trifluoroacetate

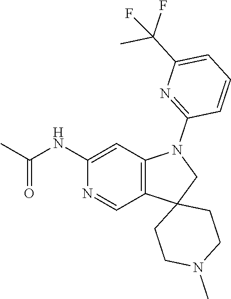 [SMILES: CC(=O)Nc1cc2c(cn1)C1(CCN(C)CC1)CN2c1cccc(C(C)(F)F)n1]

N-(1′-(6-(1,1-difluoroethyl)pyridin-2-yl)-1-methyl-1′,2′-dihydrospiro[piperidine-4,3′-pyrrolo[3,2-c]pyridin]-6′-yl)acetamide (124 mg) was prepared using a similar method to the one described in Example 274 from N-(1′-(6-(1,1-difluoroethyl)pyridin-2-yl)-1′,2′-dihydrospiro[piperidine-4,3′-pyrrolo[3,2-c]pyridin]-6′-yl)acetamide (Example 272). LCMS m/z=402.3 [M+H]⁺. ¹H NMR: (400 MHz, MeOH-d₄) δ: ppm 8.20-8.00 (m, 2H), 8.02 (s, 1H), 7.54 (d, 1H, J=7.3 Hz), 7.37 (d, 1H, J=8.3 Hz), 4.43 (s, 2H), 3.65 (br d, 2H, J=13.1 Hz), 3.34 (br s, 1H), 3.28 (br s, 1H), 2.97 (s, 3H), 2.40-2.30 (m, 5H), 2.30-2.20 (m, 2H), 2.08 (t, 3H, J=18.7 Hz).

### Example 277

### N-(1′-(4-methyl-6-(1-methylhexahydropyrrolo[3,4-b]pyrrol-5(1H)-yl)pyridin-2-yl)-1′,2′-dihydrospiro[cyclopropane-1,3′-pyrrolo[3,2-c]pyridin]-6′-yl)acetamide trifluoroacetate

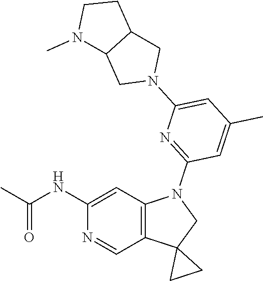 [SMILES: CC(=O)Nc1cc2c(cn1)C1(CC1)CN2c1cc(C)cc(N2CC3CCN(C)C3C2)n1]

A solution of N-(1′-(6-(hexahydropyrrolo[3,4-b]pyrrol-5(1H)-yl)-4-methylpyridin-2-yl)-1′,2′-dihydrospiro[cyclopropane-1,3′-pyrrolo[3,2-c]pyridin]-6′-yl)acetamide (Example 210, 55 mg, 135.97 μmol) and formaldehyde (12.3 M, 221.09 μL) (37% in water) in MeOH (1 mL) was stirred at rt for 15 min. Na(OAc)₃BH (144 mg, 680 μmol) was added and the reaction mixture was stirred at rt overnight. The reaction mixture was concentrated in vacuo, the crude was dissolved in DMSO and purified by HPLC (Waters XSelect CSH Prep C18 5 um OBD 30×100 mm; MeCN in water, modifier 0.1% TFA to give N-(1′-(4-methyl-6-(1-methylhexahydropyrrolo[3,4-b]pyrrol-5(1H)-yl)pyridin-2-yl)-1′,2′ -dihydrospiro[cyclopropane-1,3′-pyrrolo[3,2-c]pyridin]-6′-yl)acetamide (4.5 mg, 8% yield) as a white powder. LCMS m/z=419.3 [M+H]⁺

### Example 278

### 3-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)-1,1-dimethylurea

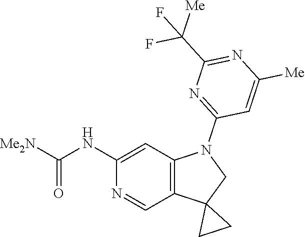 [SMILES: Cc1cc(N2CC3(CC3)c3cnc(NC(=O)N(C)C)cc32)nc(C(C)(F)F)n1]

To a vial was added 6′-chloro-1′-(2-(1,1-difluoroethyl)-6-methylpyrimidin-4-yl)-1′,2′-dihydrospiro[cyclopropane-1,3′-pyrrolo[3,2-c]pyridine] (Preparation 73, 100 mg, 0.297 mmol), 1,1-dimethylurea (130 mg, 1.48 mmol), Cs₂CO₃ (290 mg, 0.89 mmol) and BrettPhos Pd G3 (26.92 mg, 0.03 mmol) and the reaction mixture stirred overnight at 90° C. The reaction mixture was evaporated to dryness in vacuo and the residue purified by reversed phase chromatography (C-18) to yield 3-(1′-(2-(1,1-difluoroethyl)-6-methylpyrimidin-4-yl)-1′,2′-dihydrospiro[cyclopropane-1,3′-pyrrolo[3,2-c]pyridin]-6′-yl)-1,1-dimethylurea (20.7 mg, 18%). LCMS m/z=389 [M+H]⁺. 1H NMR (600 MHz, DMSO-d₆) δ ppm: 10.10-9.90 (m, 1H), 8.40-8.30 (m, 1H), 7.70 (s, 1H), 7.06 (s, 1H), 4.40-4.30 (m, 2H), 3.10-3.00 (m, 6H), 2.60-2.50 (m, 3H), 2.13 (s, 3H), 1.30-1.20 (m, 4H).

### Example 279

### N-(1′-(6-(1-fluorocyclopropyl)-4-methylpyridin-2-yl)-1′,2′-dihydrospiro[cyclopropane-1,3′-pyrrolo[3,2-c]pyridin]-6′-yl)acetamide

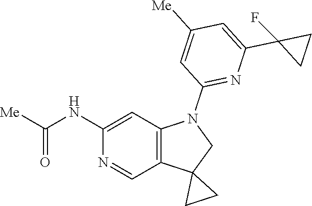 [SMILES: CC(=O)Nc1cc2c(cn1)C1(CC1)CN2c1cc(C)cc(C2(F)CC2)n1]

N-(1′-(6-Bromo-4-methylpyridin-2-yl)-1′,2′-dihydrospiro[cyclopropane-1,3′-pyrrolo[3,2-c]pyridin]-6′-yl)acetamide (Preparation 157, 121 mg, 0.325 mmol), 1,3-dioxoisoindolin-2-yl 1-fluorocyclopropane-1-carboxylate (Preparation 166, 80.9 mg, 0.325 mmol), 2-(2-pyridyl)pyridine (20.3 mg, 0.13 mmol), silver nitrate (27.6 mg, 0.162 mmol), dichloro(dimethoxyethane)nickel (28.5 mg, 0.13 mmol) and 1-methylpyrrolid-2-one (3 mL) were added to a 5-mL IKA ElectraSyn 2.0 vial with a stir bar added. The vessel was sealed, constant current was applied to the working electrode (cathode (RVC) 12 mA, 2.5 equiv e-) using IKA ElectraSyn 2.0. The counter electrode (anode)=Mg. The reaction was filtered and purified by HPLC water/MeCN 10%-90% 0.1 TFA to afford N-(1′-(6-(1-fluorocyclopropyl)-4-methylpyridin-2-yl)-1′,2′-dihydrospiro[cyclopropane-1,3′-pyrrolo[3,2-c]pyridin]-6′-yl)acetamide (2.3 mg, 2% yield). LCMS m/z=353 [M+H]⁺.

### Example 280

### N-(1′-(6-(1,1-difluoroethyl)pyrimidin-4-yl)-1′,2′-dihydrospiro[cyclopropane-1,3′-pyrrolo[3,2-c]pyridin]-6′-yl)acetamide

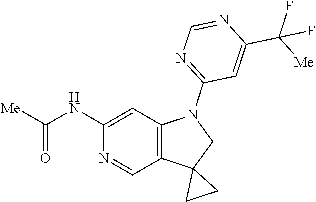 [SMILES: CC(=O)Nc1cc2c(cn1)C1(CC1)CN2c1cc(C(C)(F)F)ncn1]

To a mixture of N-(1′-(2-chloro-6-(1,1-difluoroethyl)pyrimidin-4-yl)-1′,2′-dihydrospiro[cyclopropane-1,3′-pyrrolo[3,2-c]pyridin]-6′-yl)acetamide (Example 282, 37 mg, 0.097 mmol) cyclopropanol (15 mg, 0.258 mmol), Xantphos Pd G3 (15 mg, 0.016 mmol), Cs₂CO₃ (150 mg, 0.460 mmol) was added THF (1 mL) and the mixture purged with N₂ and heated at 85° C. overnight. The reaction mixture was partitioned between EtOAc and water. The aqueous layer was extracted with EtOAc. The combined organics were dried and evaporated to dryness and the residue purified by column chromatography (24 g, 100% EtOAc) to afford N-(1′-(6-(1,1-difluoroethyl)pyrimidin-4-yl)-1′,2′-dihydrospiro[cyclopropane-1,3′-pyrrolo[3,2-c]pyridin]-6′-yl)acetamide as a white solid (27 mg, 61%). LCMS m/z=346.1 [M+H]⁺. ¹H NMR (400 MHz, MeOH-d₄) δ ppm 9.02 (s, 1H), 8.95-8.78 (m, 1H), 7.56 (s, 1H), 6.98 (s, 1H), 4.15 (s, 2H), 2.18 (s, 3H), 1.97 (t, J=19.0 Hz, 3H), 1.26-1.16 (m, 4H).

### Example 281

### Ethyl 2-(4-(6′-acetamidospiro[cyclopropane-1,3′-pyrrolo[3,2-c]pyridin]-1′(2′H)-yl)-6-methylpyrimidin-2-yl)-2,2-difluoroacetate

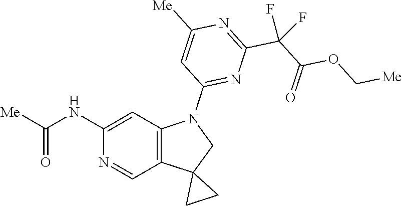 [SMILES: CCOC(=O)C(F)(F)c1nc(C)cc(N2CC3(CC3)c3cnc(NC(C)=O)cc32)n1]

A mixture of N-(1′,2′-dihydrospiro[cyclopropane-1,3′-pyrrolo[3,2-c]pyridin]-6′-yl)acetamide (Preparation 7, 150 mg, 0.738 mmol), ethyl 2-(4-chloro-6-methylpyrimidin-2-yl)-2,2-difluoroacetate (Preparation 158, 370 mg, 1.48 mmol), Cs₂CO₃ (481 mg, 1.48 mmol) and BrettPhos Pd G3 (67 mg, 0.074 mmol) in dioxane (6 mL) was purged with N₂ and heated at 90° C. for 1 h. The reaction mixture was diluted with EtOAc and washed with water. The combined organics were dried and concentrated and the residue purified by chromatography on silica gel (0-100% (3:1 EtOAc/EtOH)/heptane (+2% NH₄OH)) to give ethyl 2-(4-(6′-acetamidospiro[cyclopropane-1,3′-pyrrolo[3,2-c]pyridin]-1′(2′H)-yl)-6-methylpyrimidin-2-yl)-2,2-difluoroacetate (71 mg, 23%). LCMS m/z=418 [M+H]⁺. ¹H NMR: (500 MHz, CDCl₃) δ: ppm 9.50-9.10 (m, 1H), 9.10-8.90 (m, 1H), 7.50-7.40 (m, 1H), 7.00-6.80 (m, 1H), 4.50-4.40 (m, 2H), 4.21 (s, 2H), 2.59 (s, 3H), 2.40-2.30 (m, 3H), 1.35 (t, 3H, J=7.1 Hz), 1.23 (br d, 4H, J=2.1 Hz).

### Example 282

### N-(1′-(2-chloro-6-(1,1-difluoroethyl)pyrimidin-4-yl)-1′,2′-dihydrospiro[cyclopropane-1,3′-pyrrolo[3,2-c]pyridin]-6′-yl)acetamide

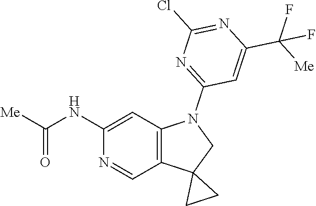 [SMILES: CC(=O)Nc1cc2c(cn1)C1(CC1)CN2c1cc(C(C)(F)F)nc(Cl)n1]

A mixture of N-(1′,2′-dihydrospiro[cyclopropane-1,3′-pyrrolo[3,2-c]pyridin]-6′-yl)acetamide (Preparation 7, 335 mg, 1.65 mmol), NaOtBu (475 mg, 4.94 mmol), 2,4-dichloro-6-(1,1-difluoroethyl)pyrimidine (Preparation 22, 678 mg, 3.18 mmol), Xantphos Pd G3 (82.3 mg, 0.82 mmol, 95% purity) and Xantphos (47.7 mg, 0.082 mmol) in dioxane (20 mL) was purged with N₂ and was then stirred at rt for 2.5 h. The reaction was partitioned between EtOAc and water. The aqueous layer was extracted with minimal volume of EtOAc. The combined organics were washed with brine, dried (MgSO₄) and evaporated to dryness under reduced pressure. The residue was purified by chromatography on silica gel (40 g, 50-100% EtOAc/heptane) to afford N-(1′-(2-chloro-6-(1,1-difluoroethyl)pyrimidin-4-yl)-1′,2′-dihydrospiro[cyclopropane-1,3′-pyrrolo[3,2-c]pyridin]-6′-yl)acetamide as a light brown solid (130 mg, 20%). LCMS m/z=380.1 [M+H]⁺. ¹H NMR (400 MHz, MeOH-d₄) δ ppm 8.99 (br s, 1H), 7.65 (d, J=1.0 Hz, 1H), 6.98 (s, 1H), 4.21 (s, 2H), 2.18 (s, 3H), 1.95 (t, J=18.8 Hz, 3H), 1.27-1.20 (m, 4H)

### Example 283

### N-(1′-(6-chloro-2-(2-fluoropropan-2-yl)pyrimidin-4-yl)-1′,2′-dihydrospiro[cyclopropane-1,3′-pyrrolo[3,2-c]pyridin]-6′-yl)acetamide

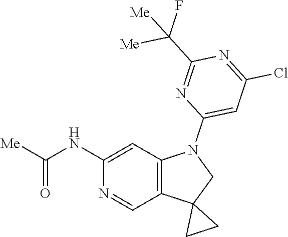 [SMILES: CC(=O)Nc1cc2c(cn1)C1(CC1)CN2c1cc(Cl)nc(C(C)(C)F)n1]

To N-(1′,2′-dihydrospiro[cyclopropane-1,3′-pyrrolo[3,2-c]pyridin]-6′-yl)acetamide (Preparation 7, 17.5 mg, 0.086 mmol), 4,6-dichloro-2-(2-fluoropropan-2-yl)pyrimidine (18 mg, 0.086 mmol) and K₃PO₄ (39 mg, 0.184 mmol) was added DMF (1 mL) and the mixture stirred at 70° C. overnight. The reaction mixture was partitioned between EtOAc and water. The aqueous layer was extracted with EtOAc and the combined organics washed with brine, dried (MgSO₄) and evaporated to dryness under reduced pressure. The residue was purified by column chromatography (50-100% EtOAc/heptane) to afford N-(1′-(6-chloro-2-(2-fluoropropan-2-yl)pyrimidin-4-yl)-1′,2′-dihydrospiro[cyclopropane-1,3′-pyrrolo[3,2-c]pyridin]-6′-yl)acetamide (3.3 mg, 10%). LCMS m/z=376.2 [M+H]⁺. ¹H NMR (400 MHz, CDCl₃) δ ppm 10.62 (br s, 1H), 9.41 (s, 1H), 7.38 (s, 1H), 6.69 (s, 1H), 4.22 (s, 2H), 2.34 (s, 3H), 1.94 (s, 3H), 1.89 (s, 3H), 1.37-1.25 (m, 5H).

### Example 284

### N-(1′-(6-(cyclopropylamino)-2-(1,1-difluoroethyl)pyrimidin-4-yl)-1′,2′-dihydrospiro[cyclopropane-1,3′-pyrrolo[3,2-c]pyridin]-6′-yl)acetamide trifluoroacetate

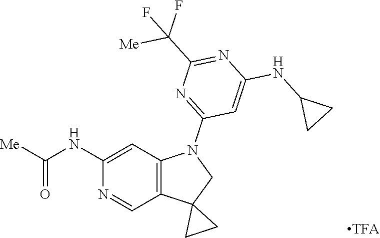 [SMILES: CC(=O)Nc1cc2c(cn1)C1(CC1)CN2c1cc(NC2CC2)nc(C(C)(F)F)n1]

To a vial was added N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 84 mg, 0.221 mmol), cyclopropanamine (63.1 mg, 1.11 mmol) and DMF (3 mL) and the mixture stirred at 80° C. overnight. The reaction mixture was diluted with EtOAc, washed with NaHCO₃, H₂O, and brine. The organics were dried (Na₂SO₄) and evaporated to dryness in vacuo. The residue was purified by HPLC (Column: Waters Sunfire OBD C18 PREP 19×100 mm, 5 um, Modifier: 0.1% TFA (v/v) conc. H₂O/MeCN (95/5 to 5/95 over 8 min) to afford the desired compound (24.3 mg, 27%). LCMS m/z=401 [M+H]⁺. 1H NMR (400 MHz, DMSO-d₆) δ ppm: 11.40-10.80 (m, 1H), 8.60-8.30 (m, 1H), 8.10-8.00 (m, 1H), 7.70-7.60 (m, 1H), 6.10-5.90 (m, 1H), 4.30-4.10 (m, 2H), 2.20-2.10 (m, 3H), 2.10-2.00 (m, 3H), 1.30-1.20 (m, 4H), 0.90-0.50 (m, 4H)

### Example 285

### N-(1′-(2-(1,1-difluoroethyl)-6-(oxetan-3-ylamino)pyrimidin-4-yl)-1′,2′-dihydrospiro[cyclopropane-1,3′-pyrrolo[3,2-c]pyridin]-6′-yl)acetamide

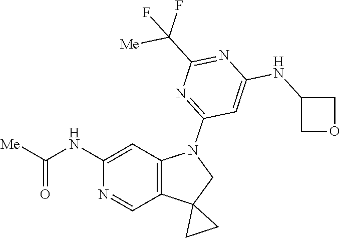 [SMILES: CC(=O)Nc1cc2c(cn1)C1(CC1)CN2c1cc(NC2COC2)nc(C(C)(F)F)n1]

The title compound was prepared, 13.3 mg, 14%, from N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155) and oxetan-3-amine using an analogous method as described for Example 284. LCMS m/z=417 [M+H]⁺. 1H NMR (400 MHz, DMSO-d₆) δ ppm: 10.30-10.20 (m, 1H), 8.90-8.80 (m, 1H), 8.30-8.20 (m, 1H), 7.70-7.60 (m, 1H), 4.90-4.80 (m, 2H), 4.60-4.50 (m, 2H), 4.10-4.00 (m, 2H), 2.10-2.20 (m, 6H), 1.20-1.10 (m, 4H)

### Example 286

### N-(1′-(2-(2-hydroxy-2-methylpropoxy)-6-methylpyrimidin-4-yl)-1′,2′-dihydrospiro[cyclopropane-1,3′-pyrrolo[3,2-c]pyridin]-6′-yl)acetamide

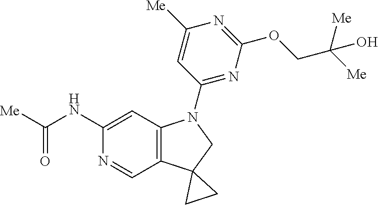 [SMILES: CC(=O)Nc1cc2c(cn1)C1(CC1)CN2c1cc(C)nc(OCC(C)(C)O)n1]

A mixture of N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 60 mg, 0.182 mmol), 2-methylpropane-1,2-diol (164 mg, 1.82 mmol) and Cs₂CO₃ (148 mg, 0.455 mmol) in DMF (2 mL) was heated at 90° C. for 16 h. The reaction mixture was diluted with EtOAc and washed with water (3×). The combined organics were dried, concentrated and the residue purified by chromatography on silica gel (0-100% (3:1 EtOAc/EtOH+2% NH₄OH) in heptane) to give N-(1′-(2-(2-hydroxy-2-methylpropoxy)-6-methylpyrimidin-4-yl)-1′,2′-dihydrospiro[cyclopropane-1,3′-pyrrolo[3,2-c]pyridin]-6′-yl)acetamide (19 mg, 27%). LCMS m/z=384 [M+H]⁺; ¹H NMR (400 MHz, CDCl₃) δ 9.16 (s, 1H), 8.29 (br s, 1H), 7.48 (s, 1H), 6.2-6.0 (m, 1H), 4.53 (s, 2H), 4.08 (s, 2H), 3.99 (br s, 1H), 2.44 (s, 3H), 2.20 (s, 3H), 1.43 (s, 6H), 1.3-1.2 (m, 4H).

### Example 287

### N-(1′-(6-(1,1-difluoroethyl)-2-(2-methoxyethoxy)pyrimidin-4-yl)-1′,2′-dihydrospiro[cyclopropane-1,3′-pyrrolo[3,2-c]pyridin]-6′-yl)acetamide

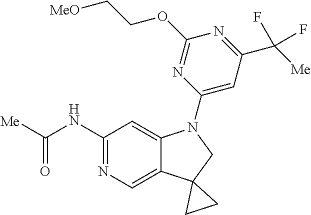 [SMILES: COCCOc1nc(N2CC3(CC3)c3cnc(NC(C)=O)cc32)cc(C(C)(F)F)n1]

To a mixture of N-(1′-(2-chloro-6-(1,1-difluoroethyl)pyrimidin-4-yl)-1′,2′-dihydrospiro[cyclopropane-1,3′-pyrrolo[3,2-c]pyridin]-6′-yl)acetamide (Example 282, 40 mg, 0.105 mmol) 2-methoxyethanol (16 mg, 0.209 mmol), Xantphos Pd G3 (15 mg, 0.016 mmol) and Cs₂CO₃ (150 mg, 0.460 mmol) was added THF (1 mL) and the mixture purged with N₂ and heated at 85° C. overnight. The reaction mixture was partitioned between EtOAc and water. The aqueous layer was extracted with EtOAc. The combined organics were dried and evaporated to dryness and the residue purified by column chromatography (50-100% EtOAc) to afford N-(1′-(6-(1,1-difluoroethyl)-2-(2-methoxyethoxy)pyrimidin-4-yl)-1′,2′-dihydrospiro[cyclopropane-1,3′-pyrrolo[3,2-c]pyridin]-6′-yl)acetamide as a white solid (27 mg, 61%). LCMS m/z=420.2 [M+H]⁺. ¹H NMR (400 MHz, CDCl₃) δ ppm 9.47 (s, 1H), 7.25 (br d, J=3.5 Hz, 1H), 6.71 (s, 1H), 4.80 (br dd, J=5.0, 3.5 Hz, 2H), 4.32 (s, 2H), 3.91 (br dd, J=5.3, 3.8 Hz, 2H), 3.49 (s, 3H), 2.38 (s, 3H), 2.08-1.93 (m, 3H), 1.43-1.38 (m, 3H), 1.33-1.27 (m, 2H)

### Example 288

### N-(1′-(2-(2-fluoroethoxy)-6-methylpyrimidin-4-yl)-1′,2′-dihydrospiro[cyclopropane-1,3′-pyrrolo[3,2-c]pyridin]-6′-yl)acetamide

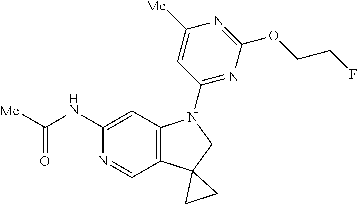 [SMILES: CC(=O)Nc1cc2c(cn1)C1(CC1)CN2c1cc(C)nc(OCCF)n1]

CPhos Pd G3 (8 mg, 0.01 mmol) and NaOtBu (19 mg, 0.2 mmol) were added to a solution of N-(1′-(2-chloro-6-methylpyrimidin-4-yl)-1′,2′-dihydrospiro[cyclopropane-1,3′-pyrrolo[3,2-c]pyridin]-6′-yl)acetamide (Preparation 153, 33 mg, 0.1 mmol) and 2-fluoroethan-1-ol (8.81 uL, 0.15 mmol) in THF (1 mL) and the reaction was stirred at 85° C. for 16 h. The reaction mixture was concentrated in vacuo and the residue purified by HPLC: Waters XSelect CSH Prep C18 5 μm OBD 19×100 mm; 5-75% MeCN/H₂O (+0.1% NH₄OH); to afford N-(1′-(2-(2-fluoroethoxy)-6-methylpyrimidin-4-yl)-1′,2′-dihydrospiro[cyclopropane-1,3′-pyrrolo[3,2-c]pyridin]-6′-yl)acetamide (9.9 mg, 28% yield) as a white solid. LCMS m/z=358 [M+H]⁺.

### Example 289

### N-(1′-(2-(1,1-difluoro-2-hydroxyethyl)-6-methylpyrimidin-4-yl)-1′,2′-dihydrospiro[cyclopropane-1,3′-pyrrolo[3,2-c]pyridin]-6′-yl)acetamide

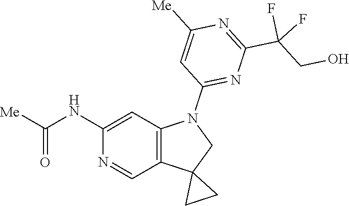 [SMILES: CC(=O)Nc1cc2c(cn1)C1(CC1)CN2c1cc(C)nc(C(F)(F)CO)n1]

To a solution of ethyl 2-(4-(6′-acetamidospiro[cyclopropane-1,3′-pyrrolo[3,2-c]pyridin]-1′(2′H)-yl)-6-methylpyrimidin-2-yl)-2,2-difluoroacetate (Example 281, 60 mg, 0.144 mmol) in EtOH (3 mL) was added NaBH₄ (11 mg, 0.287 mmol) and the mixture stirred at rt overnight. The reaction was quenched with 2M HCl and evaporated under reduced pressure. The residue was diluted with NaHCO₃/brine and extracted with EtOAc (3×). The combined organics were dried, concentrated and the residue purified by chromatography on silica gel (0-80% [(3:1 EtOAc/EtOH)+2% NH₄OH]/heptane) to give N-(1′-(2-(1,1-difluoro-2-hydroxyethyl)-6-methylpyrimidin-4-yl)-1′,2′-dihydrospiro[cyclopropane-1,3′-pyrrolo[3,2-c]pyridin]-6′-yl)acetamide (12 mg, 22%). LCMS m/z=376 [M+H]⁺. ¹H NMR (400 MHz, DMSO-d₆) δ: ppm 10.30 (s, 1H), 9.10-8.90 (m, 1H), 7.70 (s, 1H), 6.81 (s, 1H), 5.37 (t, 1H, J=6.4 Hz), 4.30-4.20 (m, 2H), 4.15 (s, 2H), 2.46 (s, 3H), 2.08 (s, 3H), 1.20-1.10 (m, 4H).

### Example 290

### N-(1′-(6-(1,1-difluoro-2-hydroxyethyl)-4-methylpyridin-2-yl)-1′,2′-dihydrospiro[cyclopropane-1,3′-pyrrolo[3,2-c]pyridin]-6′-yl)acetamide

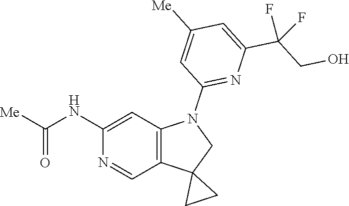 [SMILES: CC(=O)Nc1cc2c(cn1)C1(CC1)CN2c1cc(C)cc(C(F)(F)CO)n1]

N-(1′-(6-(1,1-Difluoro-2-hydroxyethyl)-4-methylpyridin-2-yl)-1′,2′-dihydrospiro[cyclopropane-1,3′-pyrrolo[3,2-c]pyridin]-6′-yl)acetamide was prepared (19 mg, 42%) from ethyl 2-(6-(6′-acetamidospiro[cyclopropane-1,3′-pyrrolo[3,2-c]pyridin]-1′(2′H)-yl)-4-methylpyridin-2-yl)-2,2-difluoroacetate (Preparation 185) using an analogous method as described for Example 289. LCMS m/z=375 [M+H]⁺. ¹H NMR (600 MHz, DMSO-d₆) δ: ppm 10.21 (br s, 1H), 8.86 (br s, 1H), 7.60 (s, 1H), 7.12 (s, 1H), 6.80 (s, 1H), 5.30 (t, 1H, J=6.2 Hz), 4.24 (dt, 2H, J=6.2, 13.8 Hz), 4.13 (s, 2H), 2.39 (s, 3H), 2.07 (s, 3H), 1.14 (br d, 4H, J=4.1 Hz).

### Example 291

### Ethyl 6-(6′-acetamidospiro[cyclopropane-1,3′-pyrrolo[3,2-c]pyridin]-1′(2′H)-yl)-2-(1,1-difluoroethyl)pyrimidine-4-carboxylate

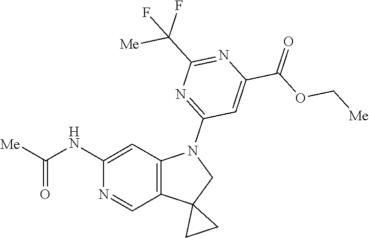 [SMILES: CCOC(=O)c1cc(N2CC3(CC3)c3cnc(NC(C)=O)cc32)nc(C(C)(F)F)n1]

Ethyl 6-(6′-acetamidospiro[cyclopropane-1,3′-pyrrolo[3,2-c]pyridin]-1′(2′H)-yl)-2-(1,1-difluoroethyl)pyrimidine-4-carboxylate was obtained, 226 mg, 22% from N-(1′-(2-(1,1-difluoroethyl)-6-(1-ethoxyvinyl)pyrimidin-4-yl)-1′,2′-dihydrospiro[cyclopropane-1,3′-pyrrolo[3,2-c]pyridin]-6′-yl)acetamide (Preparation 187), following an analogous procedure to that described in Preparation 173. LCMS m/z=418 [M+H]⁺; ¹H NMR (400 MHz, CDCl₃) δ 9.40-9.10 (m, 1H), 8.54 (br s, 1H), 7.52 (s, 1H), 7.41 (s, 1H), 4.51 (q, 2H, J=7.3 Hz), 4.23 (s, 2H), 2.30-2.20 (m, 6H), 1.47 (t, 3H, J=7.0 Hz), 1.26 (s, 4H).

### Example 292

### N-(1′-(2-(1,1-difluoroethyl)-6-(hydroxymethyl)pyrimidin-4-yl)-1′,2′-dihydrospiro[cyclopropane-1,3′-pyrrolo[3,2-c]pyridin]-6′-yl)acetamide

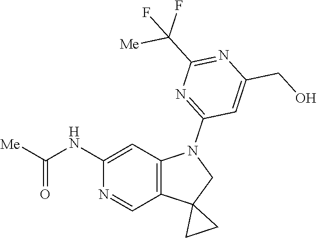 [SMILES: CC(=O)Nc1cc2c(cn1)C1(CC1)CN2c1cc(CO)nc(C(C)(F)F)n1]

N-(1′-(2-(1,1-Difluoroethyl)-6-(hydroxymethyl)pyrimidin-4-yl)-1′,2′-dihydrospiro[cyclopropane-1,3′-pyrrolo[3,2-c]pyridin]-6′-yl)acetamide was obtained, 83 mg, 54%, from ethyl 6-(6′-acetamidospiro[cyclopropane-1,3′-pyrrolo[3,2-c]pyridin]-1′(2′H)-yl)-2-(1,1-difluoroethyl)pyrimidine-4-carboxylate (Example 291), following an analogous procedure to that described in Example 289. LCMS m/z=376 [M+H]⁺.

### Example 293

### N-(1′-(2-(1,1-difluoroethyl)-6-(1-hydroxyethyl)pyrimidin-4-yl)-1′,2′-dihydrospiro[cyclopropane-1,3′-pyrrolo[3,2-c]pyridin]-6′-yl)acetamide

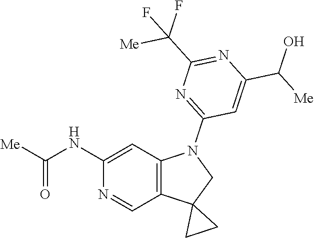 [SMILES: CC(=O)Nc1cc2c(cn1)C1(CC1)CN2c1cc(C(C)O)nc(C(C)(F)F)n1]

N-(1′-(2-(1,1-Difluoroethyl)-6-(1-hydroxyethyl)pyrimidin-4-yl)-1′,2′-dihydrospiro[cyclopropane-1,3′-pyrrolo[3,2-c]pyridin]-6′-yl)acetamide was obtained, 16 mg, 45%, from N-(1′-(6-acetyl-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 188), following an analogous procedure to that described in Example 289. LCMS m/z=390 [M+H]⁺; ¹H NMR (400 MHz, MeOH-d₄) δ 9.20-8.90 (m, 1H), 7.61 (s, 1H), 7.00 (s, 1H), 4.83-4.72 (m, 1H), 4.20 (s, 2H), 2.2-2.1 (m, 6H), 1.50 (d, 3H, J=6.8 Hz), 1.30-1.20 (m, 4H).

### Example 294

### 6-(6′-acetamidospiro[cyclopropane-1,3′-pyrrolo[3,2-c]pyridin]-1′(2′H)-yl)-2-(1,1-difluoroethyl)pyrimidine-4-carboxylic acid

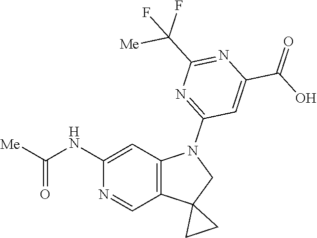 [SMILES: CC(=O)Nc1cc2c(cn1)C1(CC1)CN2c1cc(C(=O)O)nc(C(C)(F)F)n1]

Ethyl 6-(6′-acetamidospiro[cyclopropane-1,3′-pyrrolo[3,2-c]pyridin]-1′(2′H)-yl)-2-(1,1-difluoroethyl)pyrimidine-4-carboxylate (Example 291, 25 mg, 0.06 mmol) was dissolved in a mixture of THF/EtOH/H₂O (3/2/1, 1.2 mL) and aqueous solution of LiOH (1 M, 0.3 mL) added and the reaction mixture stirred at rt for 1 h. The reaction mixture was evaporated to dryness, the residue was diluted with MeOH and the solid removed by filtration. The filtrate was evaporated to dryness and the residue purified by HPLC to give 6-(6′-acetamidospiro[cyclopropane-1,3′-pyrrolo[3,2-c]pyridin]-1′(2′H)-yl)-2-(1,1-difluoroethyl)pyrimidine-4-carboxylic acid (11 mg, 47%). LCMS m/z=390 [M+H]⁺.

### Example 295

### N-(1′-(2-(1,1-difluoroethyl)-6-(3-hydroxypropyl)pyrimidin-4-yl)-1′,2′-dihydrospiro[cyclopropane-1,3′-pyrrolo[3,2-c]pyridin]-6′-yl)acetamide

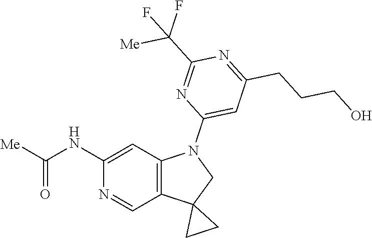 [SMILES: CC(=O)Nc1cc2c(cn1)C1(CC1)CN2c1cc(CCCO)nc(C(C)(F)F)n1]

Step 1. To a vial was added N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 100 mg, 0.263 mmol), prop-2-yn-1-ol (17.7 mg, 0.316 mmol), DIPEA (102 mg, 0.790 mmol), Pd(PPh₃)Cl₂ (18.5 mg, 0.026 mmol) and DMF (2 mL) and the reaction mixture degassed under N₂ and stirred overnight at 70° C. The mixture was diluted with EtOAc, washed with NaHCO₃, H₂O, and brine. The combined organics were dried (Na₂SO₄) and evaporated to dryness in vacuo.

Step 2. The compound of Step 1 was dissolved in EtOAc/EtOH (3:1) and AcOH (10 mL) and Pd/C (18.65 mg, 0.018 mmol, 10% purity) added. The reaction mixture was degassed for 10 min under an atmosphere of H₂ (balloon) and stirred at rt overnight. The reaction mixture was filtered and the filtrate purified by HPLC (Column: Waters Sunfire OBD C18 PREP 19×100 mm, 5 um, Modifier: 0.1% TFA (v/v) conc. H₂O/MeCN (95/5 to 5/95 over 8 min) to afford the title compound (0.8 mg, 1%). LCMS m/z=404 [M+H]⁺.

### Example 296

### N-(1′-(2-(1,1-difluoroethyl)-6-(4,5-dihydrofuran-3-yl)pyrimidin-4-yl)-1′,2′-dihydrospiro[cyclopropane-1,3′-pyrrolo[3,2-c]pyridin]-6′-yl)acetamide trifluoroacetate

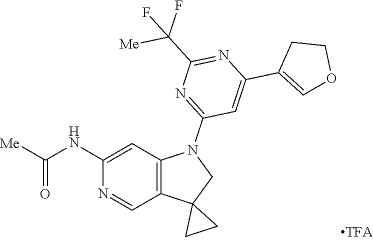 [SMILES: CC(=O)Nc1cc2c(cn1)C1(CC1)CN2c1cc(C2=COCC2)nc(C(C)(F)F)n1]

To a vial was added N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 20 mg, 0.053 mmol), 2-(2,3-dihydrofuran-4-yl)-4,4,5,5-tetramethyl-1,3,2-dioxaborolane (12.4 mg, 0.063 mmol), Pd(dppf)Cl₂; DCM (5.16 mg, 6.32 mmol), Cs₂CO₃ (51.5 mg, 0.158 mmol) and DMF (0.8 mL). The reaction mixture was degassed under N₂ and stirred at 80° C. overnight. The reaction mixture was diluted with EtOAc, washed with NaHCO₃, H₂O and brine. The combined organics were dried (Na₂SO₄) and evaporated to dryness in vacuo. The residue was purified by HPLC (Column: Waters Sunfire OBD C18 PREP 19×100 mm, 5 um, Modifier: 0.1% TFA (v/v) conc. H₂O/MeCN (95/5 to 5/95 over 8 min) to provide N-(1′-(2-(1,1-difluoroethyl)-6-(4,5-dihydrofuran-3-yl)pyrimidin-4-yl)-1′,2′-dihydrospiro[cyclopropane-1,3′-pyrrolo[3,2-c]pyridin]-6′-yl)acetamide trifluoroacetate, 6.4 mg, 29%. LCMS m/z=414 [M+H]⁺.

### Example 297

### N-(1′-(2-(1,1-difluoroethyl)-6-(tetrahydrofuran-3-yl)pyrimidin-4-yl)-1′,2′-dihydrospiro[cyclopropane-1,3′-pyrrolo[3,2-c]pyridin]-6′-yl)acetamide trifluoroacetate

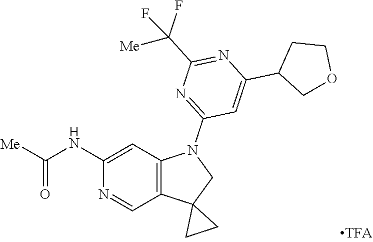 [SMILES: CC(=O)Nc1cc2c(cn1)C1(CC1)CN2c1cc(C2CCOC2)nc(C(C)(F)F)n1]

N-(1′-(2-(1,1-Difluoroethyl)-6-(tetrahydrofuran-3-yl)pyrimidin-4-yl)-1′,2′-dihydrospiro[cyclopropane-1,3′-pyrrolo[3,2-c]pyridin]-6′-yl)acetamide trifluoroacetate was obtained, 17 mg, 77% from N-(1′-(2-(1,1-difluoroethyl)-6-(4,5-dihydrofuran-3-yl)pyrimidin-4-yl)-1′,2′-dihydrospiro[cyclopropane-1,3′-pyrrolo[3,2-c]pyridin]-6′-yl)acetamide trifluoroacetate (Example 296), following the procedure described in Example 295, step 2. LCMS m/z=416 [M+H]⁺. 1H NMR (600 MHz, DMSO-d₆) δ ppm: 11.00-10.70 (m, 1H), 8.80-8.70 (m, 1H), 7.72 (s, 1H), 7.00-6.90 (m, 1H), 4.30-4.20 (m, 2H), 4.10-4.00 (m, 1H), 4.00-3.90 (m, 1H), 3.90-3.80 (m, 2H), 2.40-2.30 (m, 1H), 2.20-2.10 (m, 6H), 2.40-2.30 (m, 1H), 1.30-1.10 (m, 4H)

### Example 298

### N-(1′-(2-(1,1-difluoroethyl)-6-((methylamino)methyl)pyrimidin-4-yl)-1′,2′-dihydrospiro[cyclopropane-1,3′-pyrrolo[3,2-c]pyridin]-6′-yl)acetamide

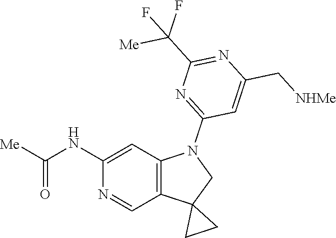 [SMILES: CNCc1cc(N2CC3(CC3)c3cnc(NC(C)=O)cc32)nc(C(C)(F)F)n1]

A mixture of N-(1′-(6-(chloromethyl)-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 189, 30 mg, 0.076 mmol) and methanamine (2M in THF, 2 mL) was stirred at 25° C. overnight. The reaction mixture was diluted with EtOAc and washed with brine. The organic layer was dried and concentrated to give N-(1′-(2-(1,1-difluoroethyl)-6-((methylamino)methyl)pyrimidin-4-yl)-1′,2′-dihydrospiro[cyclopropane-1,3′-pyrrolo[3,2-c]pyridin]-6′-yl)acetamide (29 mg, 98%). LCMS m/z=389 [M+H]⁺. 1H NMR (400 MHz, CDCl₃) δ ppm 9.06 (br s, 1H), 8.10-7.80 (m, 1H), 7.52 (s, 1H), 6.90-6.80 (m, 1H), 4.14 (s, 2H), 3.93 (s, 2H), 2.56 (s, 3H), 2.30-2.20 (m, 6H), 1.21-1.18 (m, 4H).

### Example 299

### N-(1′-(6-(azetidin-1-ylmethyl)-2-(1,1-difluoroethyl)pyrimidin-4-yl)-1′,2′-dihydrospiro[cyclopropane-1,3′-pyrrolo[3,2-c]pyridin]-6′-yl)acetamide

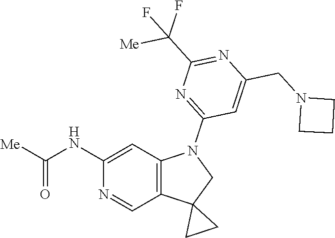 [SMILES: CC(=O)Nc1cc2c(cn1)C1(CC1)CN2c1cc(CN2CCC2)nc(C(C)(F)F)n1]

To a mixture of N-(1′-(6-(chloromethyl)-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 189, 30 mg, 0.076 mmol) and azetidine hydrochloride (107 mg, 1.14 mmol) in DMF (1 mL) was added DIPEA (148 mg, 1.14 mmol) and K₂CO₃ (21 mg, 0.152 mmol) and the reaction mixture stirred at 25° C. overnight. The reaction mixture was diluted with EtOAc, washed with water (3×) and brine. The combined organics were dried and evaporated to dryness under reduced pressure to give N-(1′-(6-(azetidin-1-ylmethyl)-2-(1,1-difluoroethyl)pyrimidin-4-yl)-1′,2′-dihydrospiro[cyclopropane-1,3′-pyrrolo[3,2-c]pyridin]-6′-yl)acetamide (29 mg, 91%). LCMS m/z=415 [M+H]⁺.1H NMR (400 MHz, CDCl₃) δ ppm 9.20-9.00 (m, 1H), 8.00 (s, 1H), 7.52 (s, 1H), 6.80-6.90 (m, 1H), 4.16 (s, 2H), 3.81 (s, 2H), 3.43 (t, 4H, J=7.0 Hz), 2.2-2.3 (m, 8H), 1.20 (br d, 4H, J=5.3 Hz).

### Example 300

### N-(1′-(2-(1,1-difluoroethyl)-6-(1-methyl-1H-1,2,4-triazol-3-yl)pyrimidin-4-yl)-1′,2′-dihydrospiro[cyclopropane-1,3′-pyrrolo[3,2-c]pyridin]-6′-yl)acetamide

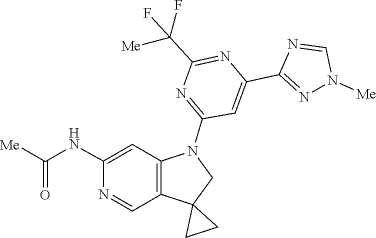 [SMILES: CC(=O)Nc1cc2c(cn1)C1(CC1)CN2c1cc(-c2ncn(C)n2)nc(C(C)(F)F)n1]

To a mixture of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 100 mg, 0.263 mmol) and PdCl₂(PPh)₃ (18 mg, 0.026 mmol) in dioxane (3 mL) at rt was added 1-methyl-3-(tributylstannyl)-1H-1,2,4-triazole (98 mg, 0.263 mmol) and the reaction mixture purged with N₂ and then heated at 100° C. for 3 h. After cooling the solid was filtered off. KF on alumina (1 g, 40 w/%) was added to the filtrate and the mixture sonicated for 1 min. The solid was filtered off, the filtrate was concentrated and the residue purified by chromatography on silica gel (20-100% (3:1 EtOAc/EtOH)/heptane (+2% NH₄OH)) to give N-(1′-(2-(1,1-difluoroethyl)-6-(1-methyl-1H-1,2,4-triazol-3-yl)pyrimidin-4-yl)-1′,2′-dihydrospiro[cyclopropane-1,3′-pyrrolo[3,2-c]pyridin]-6′-yl)acetamide (38 mg, 34%). LCMS m/z=427 [M+H]⁺.1H NMR (400 MHz, CDCl₃) δ ppm 9.22 (br s, 1H), 8.23 (br s, 1H), 8.16 (s, 1H), 7.52 (s, 1H), 7.50-7.40 (m, 1H), 4.23 (s, 2H), 4.06 (s, 3H), 2.40-2.20 (m, 6H), 1.20-1.10 (m, 4H).

### Example 301

### N-(1′-(2-(1,1-difluoroethyl)-6-(1-ethyl-5-fluoro-1H-pyrazol-4-yl)pyrimidin-4-yl)-1′,2′-dihydrospiro[cyclopropane-1,3′-pyrrolo[3,2-c]pyridin]-6′-yl)acetamide

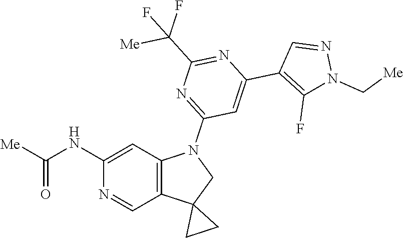 [SMILES: CCn1ncc(-c2cc(N3CC4(CC4)c4cnc(NC(C)=O)cc43)nc(C(C)(F)F)n2)c1F]

A mixture of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 100 mg, 0.263 mmol), Cs₂CO₃ (2M aqueous, 0.263 mL), 1-ethyl-5-fluoro-4-(4,4,5,5-tetramethyl-1,3,2-dioxaborolan-2-yl)-1H-pyrazole and Pd(dppf)Cl₂·DCM (22 mg, 0.026 mmol) in dioxane (3 mL) was purged with N₂ and then heated at 90° C. for 3 h. After cooling, the solid was filtered off and the filtrate evaporated to dryness. The residue was purified by chromatography on silica gel (0-80% (3:1 EtOAc/EtOH)/heptane (+2% NH₄OH)). The residue was triturated with MeCN and the solid collected and dried to afford N-(1′-(2-(1,1-difluoroethyl)-6-(1-ethyl-5-fluoro-1H-pyrazol-4-yl)pyrimidin-4-yl)-1′,2′-dihydrospiro[cyclopropane-1,3′-pyrrolo[3,2-c]pyridin]-6′-yl)acetamide (58 mg, 48%). LCMS m/z=458 [M+H]⁺. 1H NMR (400 MHz, CDCl₃) δ ppm 9.30-9.00 (m, 1H), 8.50-8.20 (m, 1H), 8.09 (d, 1H, J=3.0 Hz), 7.50 (s, 1H), 6.90-6.70 (m, 1H), 4.20-4.10 (m, 4H), 2.30-2.20 (m, 6H), 1.51 (t, 3H, J=7.3 Hz), 1.22 (s, 4H).

### Example 302

### N-(1′-(6-(1-cyanoethyl)-2-(1,1-difluoroethyl)pyrimidin-4-yl)-1′,2′-dihydrospiro[cyclopropane-1,3′-pyrrolo[3,2-c]pyridin]-6′-yl)acetamide

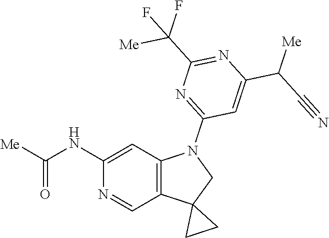 [SMILES: CC(=O)Nc1cc2c(cn1)C1(CC1)CN2c1cc(C(C)C#N)nc(C(C)(F)F)n1]

To a solution of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 150 mg, 0.395 mmol) and ethyl 2-cyanopropanoate (104 mg, 0.790 mmol) in DMF (3 mL) was added NaOtBu (76 mg, 0.79 mmol) at rt and the reaction mixture heated at 80° C. for 4 h. The mixture was diluted with EtOAc and washed with water (5×). The organic layer was dried and evaporated to dryness under reduced pressure and the residue purified by chromatography on silica gel (0-80% (3:1 EtOAc/EtOH)/heptane (+2% NH₄OH)) to give N-(1′-(6-(1-cyanoethyl)-2-(1,1-difluoroethyl)pyrimidin-4-yl)-1′,2′-dihydrospiro[cyclopropane-1,3′-pyrrolo[3,2-c]pyridin]-6′-yl)acetamide (52 mg, 33%). LCMS m/z=399 [M+H]⁺; 1H NMR (400 MHz, CDCl₃) δ ppm 11.32 (br d, 1H, J=3.5 Hz), 9.51 (s, 1H), 7.34 (s, 1H), 7.05 (s, 1H), 4.33 (s, 2H), 4.20-4.10 (m, 1H), 2.36 (s, 3H), 2.20 (t, 3H, J=18.8 Hz), 1.79 (d, 3H, J=7.5 Hz), 1.40-1.30 (m, 4H).

### Example 303

### N-(1′-(6-(cyanomethyl)-2-(1,1-difluoroethyl)pyrimidin-4-yl)-1′,2′-dihydrospiro[cyclopropane-1,3′-pyrrolo[3,2-c]pyridin]-6′-yl)acetamide

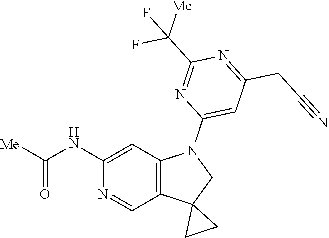 [SMILES: CC(=O)Nc1cc2c(cn1)C1(CC1)CN2c1cc(CC#N)nc(C(C)(F)F)n1]

A mixture of ethyl 2-(6-(6′-acetamidospiro[cyclopropane-1,3′-pyrrolo[3,2-c]pyridin]-1′(2′H)-yl)-2-(1,1-difluoroethyl)pyrimidin-4-yl)-2-cyanoacetate (Preparation 190, 100 mg, 0.219 mmol) and 4-methylbenzenesulfonic acid hydrate (42 mg, 0.219 mmol) in DMSO (2 mL) was heated at 110° C. for 24 h. The reaction mixture was diluted with EtOAc and washed with water (3×) and brine. The combined organics were dried, concentrated and the residue purified by chromatography on silica gel (0-100% (3:1 EtOAc/EtOH)/heptane (+2% NH₄OH)) to give N-(1′-(6-(cyanomethyl)-2-(1,1-difluoroethyl)pyrimidin-4-yl)-1′,2′-dihydrospiro[cyclopropane-1,3′-pyrrolo[3,2-c]pyridin]-6′-yl)acetamide (11 mg, 13%). LCMS m/z=385 [M+H]⁺; 1H NMR (500 MHz, CDCl₃) δ ppm 9.30-9.00 (m, 2H), 7.48 (s, 1H), 6.90-6.80 (m, 1H), 4.18 (s, 2H), 3.98 (s, 2H), 2.30-2.20 (m, 6H), 1.30-1.20 (m, 4H).

### Example 304

### N-(1′-(2-(1,1-difluoropropyl)-6-methylpyrimidin-4-yl)-1′,2′-dihydrospiro[cyclopropane-1,3′-pyrrolo[3,2-c]pyridin]-6′-yl)acetamide trifluoroacetate

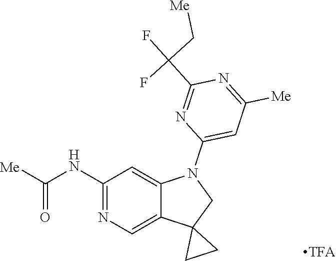 [SMILES: CCC(F)(F)c1nc(C)cc(N2CC3(CC3)c3cnc(NC(C)=O)cc32)n1]

To a solution of N-(1′-(6-chloro-2-(1,1-difluoropropyl)pyrimidin-4-yl)-1′,2′-dihydrospiro[cyclopropane-1,3′-pyrrolo[3,2-c]pyridin]-6′-yl)acetamide (Preparation 186, 24 mg, 62 μmol) in dioxane (1 mL) and water (0.1 mL) was added 2,4,6-trimethyl-1,3,5,2,4,6-trioxatriborinane (35 μL, 120 μmol), K₂CO₃ (17 mg, 120 μmol) and Pd(PPh₃)₄(7 mg, 6 μmol). The mixture was purged with N₂ and then stirred at 100° C. for 16 h. After cooling to rt, the reaction was quenched by addition of H₂O and extracted with EtOAc (2×). The combined organics were concentrated under vacuum and the residue purified by reversed phase HPLC (Sunfire Prep C18 OBD, 100×30 mm, 5 mm; 5-55% MeCN/H₂O (0.1% TFA)) to afford N-(1′-(2-(1,1-difluoropropyl)-6-methylpyrimidin-4-yl)-1′,2′-dihydrospiro[cyclopropane-1,3′-pyrrolo[3,2-c]pyridin]-6′-yl)acetamide as a white solid (17 mg, 56% yield). LCMS m/z=374.1 [M+H]⁺. ¹H NMR (600 MHz, DMSO-d₆) δ ppm 10.74 (br s, 1H), 8.77 (br s, 1H), 7.70 (s, 1H), 6.89 (s, 1H), 4.20 (s, 2H), 2.51-2.56 (m, 2H), 2.48 (s, 3H), 2.13 (s, 3H), 1.16-1.25 (m, 4H), 0.97 (t, J=7.4 Hz, 3H).

### Example 305

### N-(1′-(2-(1,1-difluoroethyl)-6-fluoropyrimidin-4-yl)-1′,2′-dihydrospiro[cyclopropane-1,3′-pyrrolo[3,2-c]pyridin]-6′-yl)acetamide trifluoroacetate

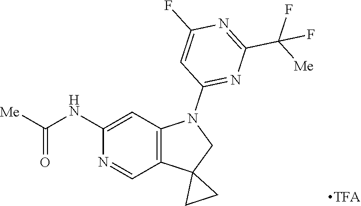 [SMILES: CC(=O)Nc1cc2c(cn1)C1(CC1)CN2c1cc(F)nc(C(C)(F)F)n1]

TBAF (1 M in THF, 100 μL, 100 μmol) was added to a solution of N-(1′-(6-chloro-2-(1,1-difluoroethyl)pyrimidin-4-yl)-1′,2′-dihydrospiro[cyclopropane-1,3′-pyrrolo[3,2-c]pyridin]-6′-yl)acetamide (Preparation 155, 19 mg, 50 μmol) in DMF (2 mL) and the mixture was heated at 140° C. for 1 h. The reaction was cooled to rt, diluted with saturated NaHCO₃, extracted with EtOAc (2×) and evaporated to dryness. The residue was purified by reversed phase HPLC (Sunfire Prep C18 OBD 100×30 mm; 5 mm; 5-60% MeCN/H₂O (+0.1% TFA)) to afford N-(1′-(2-(1,1-difluoroethyl)-6-fluoropyrimidin-4-yl)-1′,2′-dihydrospiro[cyclopropane-1,3′-pyrrolo[3,2-c]pyridin]-6′-yl)acetamide trifluoroacetate as a white solid (4 mg, 15%). LCMS m/z=364.0 [M+H]⁺. ¹H NMR (600 MHz, DMSO-d₆) δ ppm 10.55 (br s, 1H), 8.93 (br s, 1H), 7.76 (s, 1H), 6.73 (s, 1H), 4.19 (s, 2H), 2.18 (t, J=19.3 Hz, 3H), 2.06-2.14 (m, 3H), 1.15-1.28 (m, 4H).

### Example 306

### N-(1′-(4-methyl-6-(1-methyl-2-oxabicyclo[2.1.1]hexan-4-yl)pyridin-2-yl)-1′,2′-dihydrospiro[cyclopropane-1,3′-pyrrolo[3,2-c]pyridin]-6′-yl)acetamide

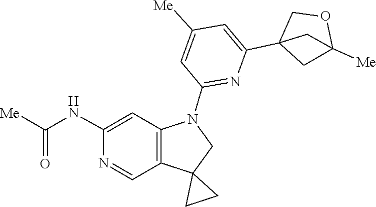 [SMILES: CC(=O)Nc1cc2c(cn1)C1(CC1)CN2c1cc(C)cc(C23COC(C)(C2)C3)n1]

A mixture of Cu(TMHD)₂ (28.8 mg, 0.067 mmol), 1,3-dioxoisoindolin-2-yl 1-methyl-2-oxabicyclo[2.1.1]hexane-4-carboxylate (Preparation 165, 123 mg, 0.429 mmol), aminosupersilane (106.6 mg, 0.268 mmol), bis[2-(2-pyridyl)phenyl]iridium(1+) 4-tert-butyl-2-(4-tert-butyl-2-pyridyl)pyridine hexafluorophosphate (1.22 mg, 1.34 mmol), N-(1′-(6-bromo-4-methylpyridin-2-yl)-1′,2′-dihydrospiro[cyclopropane-1,3′-pyrrolo[3,2-c]pyridin]-6′-yl)acetamide (Preparation 157, 50 mg, 0.134 mmol) and aqueous acetone (5 mL) was sparged with N₂ for 1 min. The mixture was transferred to a flask containing NaOAc (87.9 mg, 1.07 mmol) that had previously been dried in vacuo at 75° C. overnight. The combined mixture was purged with N₂ and sealed with parafilm and sonicated for 1 min. The reaction vessel was placed in Integrated Photoreactor (450 nm LEDs) and stirred at rt for 1 h. The acetone was removed under reduced pressure and the residue diluted with brine and extracted with EtOAc (2×). The combined organics were dried and evaporated to dryness. The residue was purified by HPLC to give N-(1′-(4-methyl-6-(1-methyl-2-oxabicyclo[2.1.1]hexan-4-yl)pyridin-2-yl)-1′,2′-dihydrospiro[cyclopropane-1,3′-pyrrolo[3,2-c]pyridin]-6′-yl)acetamide (3 mg, 60%). LCMS m/z=391 [M+H]⁺; ¹H NMR (600 MHz, DMSO-d₆) δ 11.14 (br s, 1H), 8.33 (br s, 1H), 7.58 (s, 1H), 6.97 (br s, 1H), 6.77 (br s, 1H), 4.25 (s, 2H), 4.01 (s, 2H), 2.35 (s, 3H), 2.20 (m, 3H), 2.13 (m, 2H), 1.91 (m, 2H), 1.46 (s, 3H), 1.23 (m, 4H).

### Examples 307 to 411

the compounds in the following table were prepared following a similar procedure to that described in the Examples above.

| Example | |
|---|---|
| no | Name/Structure/Data |
| 307 | 1-(1′-(2-(1,1-difluoroethyl)-6-methylpyrimidin-4-yl)-1′,2′- dihydrospiro[cyclopropane-1,3′-pyrrolo[3,2-c]pyridin]-6′-yl)-3-methylurea 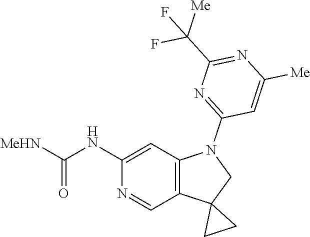 [SMILES: CNC(=O)Nc1cc2c(cn1)C1(CC1)CN2c1cc(C)nc(C(C)(F)F)n1] LCMS m/z = 375 [M + H]⁺. 1H NMR (600 MHz, DMSO-d₆) δ ppm: 8.1-8.0 (m, 1H), 7.7-7.6 (m, 1H), 7.6-7.5 (m, 1H), 7.1-7.0 (m, 1H), 4.3-4.2 (m, 2H), 2.8-2.7 (m, 3H), 2.6-2.5 (m, 3H), 2.12 (t, 3H, J = 19.1 Hz), 1.3-1.3 (m, 4H) |
| 308 | 1-(1′-(2-(1,1-difluoroethyl)-6-methylpyrimidin-4-yl)-1′,2′- dihydrospiro[cyclopropane-1,3′-pyrrolo[3,2-c]pyridin]-6′-yl)urea 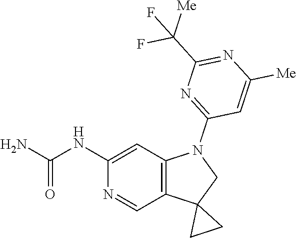 [SMILES: Cc1cc(N2CC3(CC3)c3cnc(NC(N)=O)cc32)nc(C(C)(F)F)n1] LCMS m/z = 361 [M + H]⁺. |
| 309 | 1-(1′-(2-(1,1-difluoroethyl)-6-ethylpyrimidin-4-yl)-1′,2′- dihydrospiro[cyclopropane-1,3′-pyrrolo[3,2-c]pyridin]-6′-yl)urea 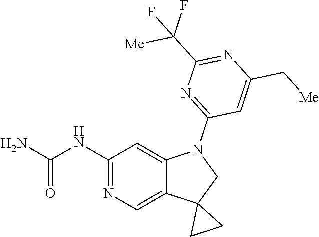 [SMILES: CCc1cc(N2CC3(CC3)c3cnc(NC(N)=O)cc32)nc(C(C)(F)F)n1] LCMS m/z = 375 [M + H]⁺. ¹H NMR (400 MHz, DMSO-d₆) δ 9.12 (s, 1H), 8.29 (s, 1H), 7.59 (s, 1H), 6.80 (s, 2H), 4.15 (s, 2H), 2.74 (q, 2H, J = 7.7 Hz), 2.14 (t, 3H, J = 19.1 Hz), 1.25 (t, 3H, J = 7.5 Hz), 1.14 (s, 4H). |
| 310 | N-(1′-(2-(1,1-difluoroethyl)-6-(5-fluoro-1-methyl-1H-pyrazol-4-yl)pyrimidin- 4-yl)-1′,2′-dihydrospiro[cyclopropane-1,3′-pyrrolo[3,2-c]pyridin]-6′- yl)acetamide 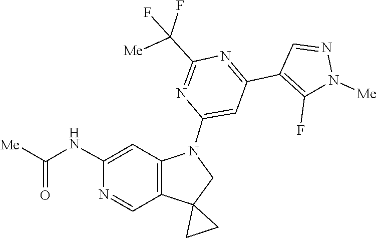 [SMILES: CC(=O)Nc1cc2c(cn1)C1(CC1)CN2c1cc(-c2cnn(C)c2F)nc(C(C)(F)F)n1] LCMS m/z = 444 [M + H]⁺; ¹H NMR (400 MHz, CDCl₃) δ 9.14 (br s, 1H), 8.9- 8.4 (m, 1H), 8.09 (d, 1H, J = 2.8 Hz), 7.46 (s, 1H), 6.9-6.7 (m, 1H), 4.20 (s, 2H), 3.84 (d, 3H, J = 1.3 Hz), 2.2-2.3 (m, 6H), 1.24 (s, 4H). |
| 311 | Methyl (1′-(2-(1,1-difluoroethyl)-6-ethylpyrimidin-4-yl)-1′,2′- dihydrospiro[cyclopropane-1,3′-pyrrolo[3,2-c]pyridin]-6′-yl)carbamate trifluoroacetate 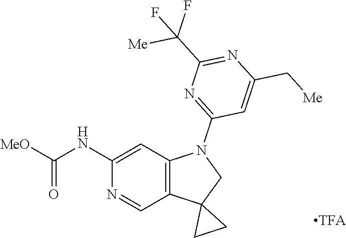 [SMILES: CCc1cc(N2CC3(CC3)c3cnc(NC(=O)OC)cc32)nc(C(C)(F)F)n1] LCMS m/z = 390.1 [M + H]⁺; ¹H NMR (600 MHz, DMSO-d₃) δ 10.18 (br s, 1H), 8.71 (s, 1H), 7.67 (s, 1H), 6.83 (s, 1H), 4.20 (s, 2H), 3.69 (s, 3H), 3.50- 3.43 (m, 2H), 2.82-2.73 (m, 2H), 2.18-2.06 (m, 3H), 1.37-1.24 (m, 3H), 1.19 (br d, J = 3.81 Hz, 4H). |
| 312 | N-(1′-(2-(2-fluoropropan-2-yl)-6-methylpyrimidin-4-yl)-1′,2′- dihydrospiro[cyclopropane-1,3′-pyrrolo[3,2-c]pyridin]-6′-yl)acetamide 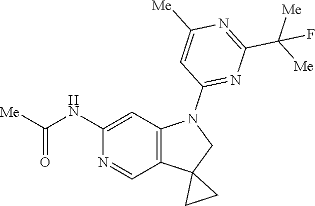 [SMILES: CC(=O)Nc1cc2c(cn1)C1(CC1)CN2c1cc(C)nc(C(C)(C)F)n1] LCMS m/z = 356.2 [M + H]⁺; ¹H NMR (400 MHz, MeOH-d₄) δ 9.04 (s, 1H), 7.61-7.54 (m, 1H), 6.63 (s, 1H), 4.14 (s, 2H), 2.49 (s, 3H), 2.17 (s, 3H), 1.92- 1.75 (m, 6H), 1.28-1.17 (m, 4H). |
| 313 | N-(1′-(6-methoxypyridin-2-yl)-1′,2′-dihydrospiro[cyclopropane-1,3′- pyrrolo[3,2-c]pyridin]-6′-yl)acetamide 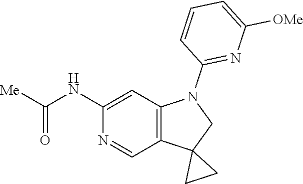 [SMILES: COc1cccc(N2CC3(CC3)c3cnc(NC(C)=O)cc32)n1] LCMS m/z = 311.0 [M + H]⁺; ¹H NMR (400 MHz, CDCl₃) δ 9.41 (br s, 1H), 9.00 (s, 1H), 7.58 (t, J = 8.0 Hz, 1H), 7.32 (s, 1H), 6.46 (d, J = 8.0 Hz, 1H), 6.31 (d, J = 8.0 Hz, 1H), 4.16 (s, 3H), 4.13 (s, 2H), 2.24 (s, 3H), 1.19 (s, 4H). |
| 314 | N-(1′-(6-ethyl-2-methylpyrimidin-4-yl)-1′,2′-dihydrospiro[cyclopropane-1,3′- pyrrolo[3,2-c]pyridin]-6′-yl)acetamide 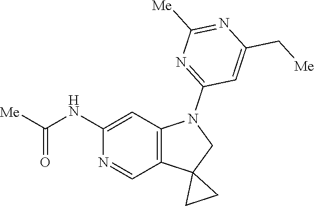 [SMILES: CCc1cc(N2CC3(CC3)c3cnc(NC(C)=O)cc32)nc(C)n1] LCMS m/z = 324.1 [M + H]⁺; ¹H NMR (400 MHz, MeOH-d₄) δ 9.14 (s, 1H), 7.54 (s, 1H), 6.51 (s, 1H), 4.09 (s, 2H), 2.69 (q, J = 7.5 Hz, 2H), 2.63 (s, 3H), 2.18 (s, 3H), 1.33-1.26 (m, 3H), 1.21-1.16 (m, 4H). |
| 315 | N-(1′-(6-(1,1-difluoro-2-methoxyethyl)-4-methylpyridin-2-yl)-1′,2′- dihydrospiro[cyclopropane-1,3′-pyrrolo[3,2-c]pyridin]-6′-yl)acetamide trifluoroacetate 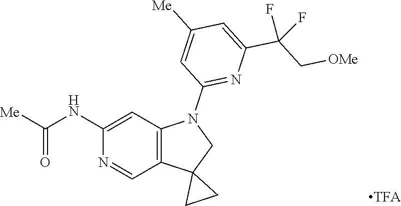 [SMILES: COCC(F)(F)c1cc(C)cc(N2CC3(CC3)c3cnc(NC(C)=O)cc32)n1] LCMS m/z = 389.1 [M + H]⁺; ¹H NMR (600 MHz, DMSO-d₆) δ 10.9-10.2 (m, 1H), 9.0-8.5 (m, 1H), 7.61 (s, 1H), 7.19 (s, 1H), 6.86 (br s, 1H), 4.30 (br t, 2H, J = 14.0 Hz), 4.18 (s, 2H), 3.34 (s, 3H), 2.40 (s, 3H), 2.11 (s, 3H), 1.2-1.1 (m, 4H). |
| 316 | N-(1′-(4-methyl-6-(1,1,2-trifluoroethyl)pyridin-2-yl)-1′,2′- dihydrospiro[cyclopropane-1,3′-pyrrolo[3,2-c]pyridin]-6′-yl)acetamide trifluoroacetate 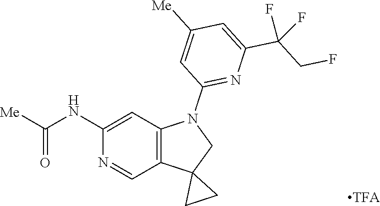 [SMILES: CC(=O)Nc1cc2c(cn1)C1(CC1)CN2c1cc(C)cc(C(F)(F)CF)n1] LCMS m/z = 377.1 [M + H]⁺; ¹H NMR (600 MHz, DMSO-d₆) δ 11.5-10.5 (m, 1H), 8.50 (br s, 1H), 7.62 (s, 1H), 7.35 (s, 1H), 7.00 (br s, 1H), 5.4-5.3 (m, 2H), 4.24 (s, 2H), 2.43 (s, 3H), 2.16 (s, 3H), 1.21 (br d, 4H, J = 6.8 Hz). |
| 317 | N-(1′-(2-(3-methoxycyclobutyl)-6-methylpyrimidin-4-yl)-1′,2′- dihydrospiro[cyclopropane-1,3′-pyrrolo[3,2-c]pyridin]-6′-yl)acetamide 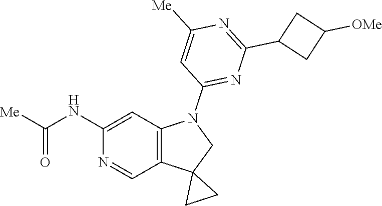 [SMILES: COC1CC(c2nc(C)cc(N3CC4(CC4)c4cnc(NC(C)=O)cc43)n2)C1] LCMS m/z = 380 [M + H]⁺; 1H NMR (600 MHz, DMSO-d₆) d ppm 10.26- 10.20 (m, 1 H), 9.05 (br s, 1 H), 7.66-7.64 (m, 1 H), 6.54 (s, 1 H), 4.11-4.08 (m, 2H), 3.90-3.85 (m, 1H), 3.33 (s, 3H), 3.17 (s, 3 H), 3.16-3.11 (m, 1H), 2.66-2.60 (m, 2H), 2.38 (s, 3H), 2.30 (m, 2H), 1.19-1.12 (m, 4H). |
| 318 | N-(1′-(6-methyl-2-(1-methyl-2-oxabicyclo[2.1.1]hexan-4-yl)pyrimidin-4-yl)- 1′,2′-dihydrospiro[cyclopropane-1,3′-pyrrolo[3,2-c]pyridin]-6′-yl)acetamide 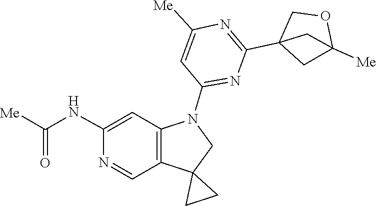 [SMILES: CC(=O)Nc1cc2c(cn1)C1(CC1)CN2c1cc(C)nc(C23COC(C)(C2)C3)n1] LCMS m/z = 392 [M + H]⁺; 1H NMR (CDCl₃, 400 MHz) δ 9.32 (s, 1H), 7.4- 7.3 (m, 1H), 6.5-6.4 (m, 1H), 4.24 (s, 2H), 4.16 (s, 2H), 2.51 (s, 3H), 2.42 (dd, 2H, J = 1.5, 4.5 Hz), 2.28 (s, 3H), 2.02 (dd, 2H, J = 1.8, 4.5 Hz), 1.59 (s, 3H), 1.3-1.2 (m, 4H). |
| 319 | N-(1′-(6-methyl-2-(2-(methylsulfonyl)ethyl)pyrimidin-4-yl)-1′,2′- dihydrospiro[cyclopropane-1,3′-pyrrolo[3,2-c]pyridin]-6′-yl)acetamide 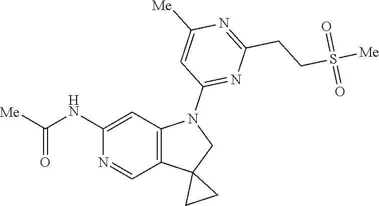 [SMILES: CC(=O)Nc1cc2c(cn1)C1(CC1)CN2c1cc(C)nc(CCS(C)(=O)=O)n1] LCMS m/z = 402 [M + H]⁺. 1H NMR (CDCl₃, 400 MHz) δ 9.17 (s, 1H), 8.7- 8.1 (m, 1H), 7.47 (s, 1H), 6.33 (s, 1H), 4.08 (s, 2H), 4.0-3.9 (m, 2H), 3.6-3.5 (m, 2H), 3.15 (s, 3H), 2.5-2.4 (m, 3H), 2.20 (s, 3H), 1.3-1.2 (m, 4H). |
| 320 | N-(1′-(2-(2-oxabicyclo[2.1.1]hexan-4-yl)-6-methylpyrimidin-4-yl)-1′,2′- dihydrospiro[cyclopropane-1,3′-pyrrolo[3,2-c]pyridin]-6′-yl)acetamide 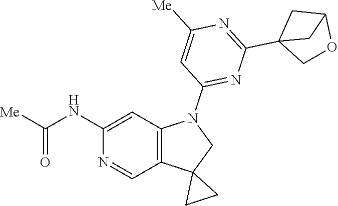 [SMILES: CC(=O)Nc1cc2c(cn1)C1(CC1)CN2c1cc(C)nc(C23COC(C2)C3)n1] LCMS m/z = 378 [M + H]⁺. 1H NMR (400 MHz, MeOH-d₄) δ 9.05 (s, 1H), 7.57 (s, 1H), 6.58 (s, 1H), 4.65 (t, 1H, J = 1.1 Hz), 4.12 (d, 4H, J = 6.3 Hz), 2.5- 2.4 (m, 5H), 2.17 (s, 3H), 2.02-2.00 (m, 2H), 1.21-1.20 (m, 4H). |
| 321 | N-(1′-(6-ethyl-2-(tetrahydrofuran-3-yl)pyrimidin-4-yl)-1′,2′- dihydrospiro[cyclopropane-1,3′-pyrrolo[3,2-c]pyridin]-6′-yl)acetamide trifluoroacetate 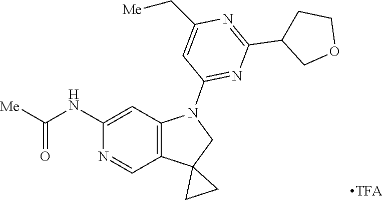 [SMILES: CCc1cc(N2CC3(CC3)c3cnc(NC(C)=O)cc32)nc(C2CCOC2)n1] LCMS m/z = 380 [M + H]⁺. 1H NMR (600 MHz, DMSO-d₆) d ppm 8.75 (br s, 1H), 7.68 (s, 1H), 6.71 (s, 1H), 4.15-4.28 (m, 3H), 4.01-3.91 (m, 1H), 3.90- 3.83 (m, 2H), 3.75-3.61 (m, 1H), 2.71 (q, J = 7.60 Hz, 2H), 2.49-2.30 (m, 2 H), 2.16 (s, 3 H), 1.27-1.21 (m, 7H). |
| 322 | N-(1′-(2-(2-oxabicyclo[2.1.1]hexan-4-yl)-6-ethylpyrimidin-4-yl)-1′,2′- dihydrospiro[cyclopropane-1,3′-pyrrolo[3,2-c]pyridin]-6′-yl)acetamide 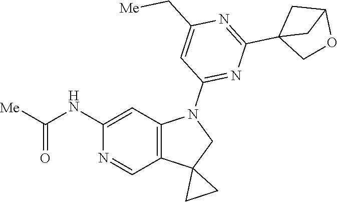 [SMILES: CCc1cc(N2CC3(CC3)c3cnc(NC(C)=O)cc32)nc(C23COC(C2)C3)n1] LCMS m/z = 392 [M + H]⁺. 1H NMR (400 MHz, MeOH-d₄) δ 9.02 (s, 1H), 7.56 (s, 1H), 6.6-6.5 (m, 1H), 4.66 (t, 1H, J = 1.0 Hz), 4.14 (d, 4H, J = 2.5 Hz), 2.74 (q, 2H, J = 7.7 Hz), 2.5-2.4 (m, 2H), 2.18 (s, 3H), 2.01-2.00 (m, 2H), 1.31 (t, 3H, J = 7.6 Hz), 1.22-1.20 (m, 4H). |
| 323 | c1nc(NC(C)=O)cc2c1C3(CC3)CN2c4ccnc(C(F)(F)COC)n4 N-(1′-(2-(1,1-difluoro-2-methoxyethyl)pyrimidin-4-yl)-1′,2′- dihydrospiro[cyclopropane-1,3′-pyrrolo[3,2-c]pyridin]-6′-yl)acetamide 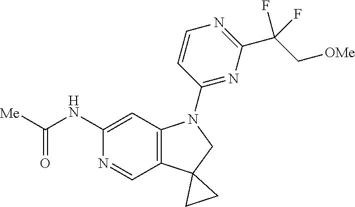 [SMILES: COCC(F)(F)c1nccc(N2CC3(CC3)c3cnc(NC(C)=O)cc32)n1] LCMS m/z = 376 [M + H]⁺. 1H NMR (400 MHz, CDCl₃) δ 9.04 (br s, 1H), 8.63 (d, 1H, J = 5.8 Hz), 8.10 (br s, 1H), 7.53 (s, 1H), 6.9-6.6 (m, 1H), 4.35 (t, 2H, J = 13.0 Hz), 4.14 (s, 2H), 3.52 (s, 3H), 2.3-2.2 (m, 3H), 1.20 (br d, 4H, J = 6.5 Hz). |
| 324 | N-(1′-(2-(1,1-difluoroethyl)-6-(3-hydroxy-3-methylpentyl)pyrimidin-4-yl)- 1′,2′-dihydrospiro[cyclopropane-1,3′-pyrrolo[3,2-c]pyridin]-6′-yl)acetamide 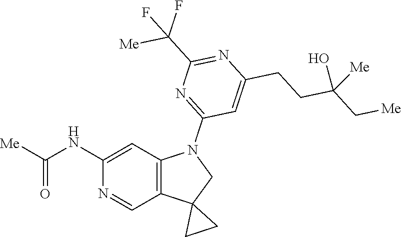 [SMILES: CCC(C)(O)CCc1cc(N2CC3(CC3)c3cnc(NC(C)=O)cc32)nc(C(C)(F)F)n1] LCMS m/z = 446 [M + H]⁺. 1H NMR (400 MHz, DMSO-d₆) δ ppm: 10.5-10.4 (m, 1H), 9.0-8.9 (m, 1H), 7.7-7.6 (m, 1H), 6.8-6.7 (m, 1H), 4.3-4.2 (m, 2H), 2.8-2.7 (m, 2H), 2.2-2.1 (m, 3H), 2.1-2.0 (m, 3H), 1.8-1.7 (m, 2H), 1.5-1.4 (m, 2H), 1.3-1.2 (m, 4H), 1.1-1.0 (m, 3H), 0.9-0.8 (m, 3H) |
| 325 | N-(1′-(6-(3-(tert-butoxy)propyl)-2-(1,1-difluoroethyl)pyrimidin-4-yl)-1′,2′- dihydrospiro[cyclopropane-1,3′-pyrrolo[3,2-c]pyridin]-6′-yl)acetamide 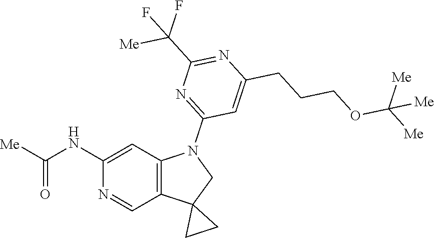 [SMILES: CC(=O)Nc1cc2c(cn1)C1(CC1)CN2c1cc(CCCOC(C)(C)C)nc(C(C)(F)F)n1] LCMS m/z = 460 [M + H]⁺. |
| 326 | N-(1′-(2-(1,1-difluoroethyl)-6-(3-methoxypropyl)pyrimidin-4-yl)-1′,2′- dihydrospiro[cyclopropane-1,3′-pyrrolo[3,2-c]pyridin]-6′-yl)acetamide 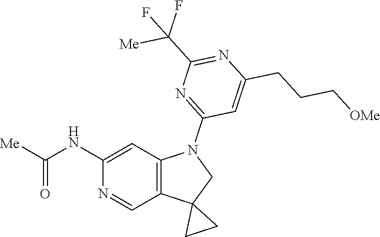 [SMILES: COCCCc1cc(N2CC3(CC3)c3cnc(NC(C)=O)cc32)nc(C(C)(F)F)n1] LCMS m/z = 418 [M + H]⁺. |
| 327 | N-(1′-(2-(1,1-difluoroethyl)-6-(3-hydroxy-3-methylbutyl)pyrimidin-4-yl)- 1′,2′-dihydrospiro[cyclopropane-1,3′-pyrrolo[3,2-c]pyridin]-6′-yl)acetamide 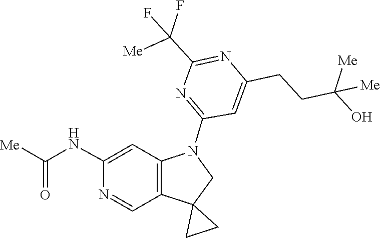 [SMILES: CC(=O)Nc1cc2c(cn1)C1(CC1)CN2c1cc(CCC(C)(C)O)nc(C(C)(F)F)n1] LCMS m/z = 432 [M + H]⁺ |
| 328 | N-(1′-(2-(1,1-difluoroethyl)-6-pentylpyrimidin-4-yl)-1′,2′- dihydrospiro[cyclopropane-1,3′-pyrrolo[3,2-c]pyridin]-6′-yl)acetamide 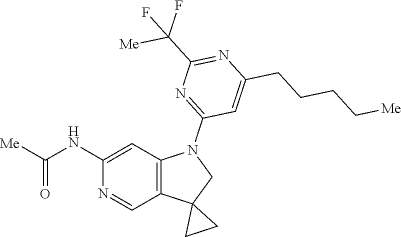 [SMILES: CCCCCc1cc(N2CC3(CC3)c3cnc(NC(C)=O)cc32)nc(C(C)(F)F)n1] LCMS m/z = 416 [M + H]⁺. |
| 329 | (Z)-N-(1′-(2-(1,1-difluoroethyl)-6-(2-ethoxyvinyl)pyrimidin-4-yl)-1′,2′- dihydrospiro[cyclopropane-1,3′-pyrrolo[3,2-c]pyridin]-6′-yl)acetamide 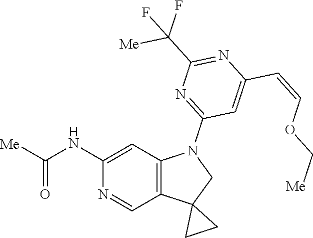 [SMILES: CCO/C=C\c1cc(N2CC3(CC3)c3cnc(NC(C)=O)cc32)nc(C(C)(F)F)n1] LCMS m/z = 416 [M + H]⁺. |
| 330 | N-(1′-(2-(1,1-difluoroethyl)-6-(2-ethoxyethyl)pyrimidin-4-yl)-1′,2′- dihydrospiro[cyclopropane-1,3′-pyrrolo[3,2-c]pyridin]-6′-yl)acetamide trifluoroacetate 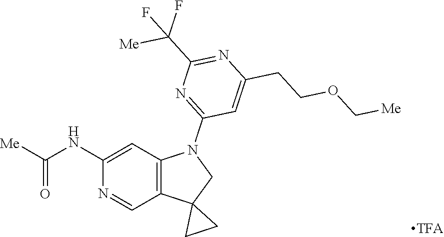 [SMILES: CCOCCc1cc(N2CC3(CC3)c3cnc(NC(C)=O)cc32)nc(C(C)(F)F)n1] LCMS m/z = 418 [M + H]⁺. 1H NMR (400 MHz, DMSO-d₆) δ ppm: 10.4-10.3 (m, 1H), 9.1-9.0 (m, 1H), 7.71 (s, 1H), 6.9-6.8 (m, 1H), 4.2-4.1 (m, 2H), 3.8- 3.7 (m, 2H), 3.5-3.4 (m, 2H), 3.0-2.9 (m, 2H), 2.2-2.1 (m, 3H), 2.1-2.0 (m, 3H), 1.3-1.2 (m, 4H), 1.1-1.0 (m, 3H) |
| 331 | N-(1′-(4-fluoro-6-methylpyrimidin-2-yl)-1′,2′-dihydrospiro[cyclopropane-1,3′- pyrrolo[3,2-c]pyridin]-6′-yl)acetamide trifluoroacetate 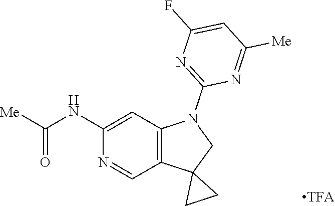 [SMILES: CC(=O)Nc1cc2c(cn1)C1(CC1)CN2c1nc(C)cc(F)n1] LCMS m/z = 314.1 [M + H]⁺. ¹H NMR (600 MHz, DMSO-d₆) δ ppm 11.14 (br s, 1 H), 8.58 (br s, 1 H), 7.64 (s, 1 H), 6.90 (br s, 1 H), 4.27 (s, 2 H), 2.53- 2.55 (m, 3 H), 2.17 (s, 3 H), 1.17-1.30 (m, 4 H). |
| 332 | N-(1′-(2-fluoro-6-methylpyrimidin-4-yl)-1′,2′-dihydrospiro[cyclopropane-1,3′- pyrrolo[3,2-c]pyridin]-6′-yl)acetamide trifluoroacetate 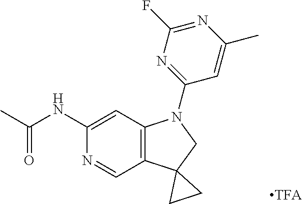 [SMILES: CC(=O)Nc1cc2c(cn1)C1(CC1)CN2c1cc(C)nc(F)n1] LCMS m/z = 314.1 [M + H]⁺. ¹H NMR (600 MHz, DMSO-d₆) δ ppm 11.46 (br s, 1H), 8.62 (br s, 1H), 7.64 (s, 1H), 5.94 (s, 1H), 4.18 (s, 2H), 2.20 (s, 3H), 2.14 (s, 3H), 1.16-1.28 (m, 4H). |
| 333 | N-(1′-(4-methyl-6-(2,2,2-trifluoroethoxy)pyridin-2-yl)-1′,2′- dihydrospiro[cyclopropane-1,3′-pyrrolo[3,2-c]pyridin]-6′-yl)acetamide trifluoroacetate 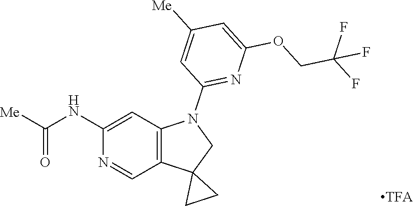 [SMILES: CC(=O)Nc1cc2c(cn1)C1(CC1)CN2c1cc(C)cc(OCC(F)(F)F)n1] LCMS m/z = 393 [M + H]⁺. |
| 334 | N-(1′-(6-isopropoxy-4-methylpyridin-2-yl)-1′,2′-dihydrospiro[cyclopropane- 1,3′-pyrrolo[3,2-c]pyridin]-6′-yl)acetamide trifluoroacetate 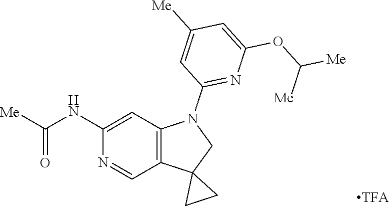 [SMILES: CC(=O)Nc1cc2c(cn1)C1(CC1)CN2c1cc(C)cc(OC(C)C)n1] LCMS m/z = 353 [M + H]⁺. |
| 335 | N-(1′-(6-ethoxy-4-methylpyridin-2-yl)-1′,2′-dihydrospiro[cyclopropane-1,3′- pyrrolo[3,2-c]pyridin]-6′-yl)acetamide trifluoroacetate 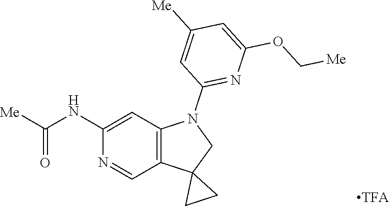 [SMILES: CCOc1cc(C)cc(N2CC3(CC3)c3cnc(NC(C)=O)cc32)n1] LCMS m/z = 339 [M + H]⁺. |
| 336 | N-(1′-(6-(2-methoxy-2-methylpropoxy)-4-methylpyridin-2-yl)-1′,2′- dihydrospiro[cyclopropane-1,3′-pyrrolo[3,2-c]pyridin]-6′-yl)acetamide trifluoroacetate 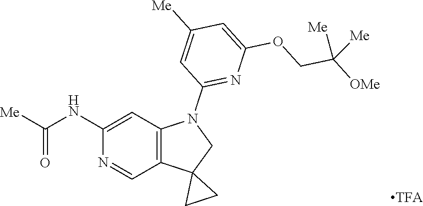 [SMILES: COC(C)(C)COc1cc(C)cc(N2CC3(CC3)c3cnc(NC(C)=O)cc32)n1] LCMS m/z = 397 [M + H]⁺. |
| 337 | N-(1′-(6-((3-hydroxycyclopentyl)oxy)-4-methylpyridin-2-yl)-1′,2′- dihydrospiro[cyclopropane-1,3′-pyrrolo[3,2-c]pyridin]-6′-yl)acetamide trifluoroacetate 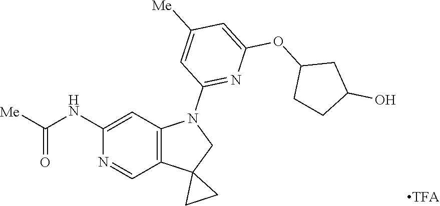 [SMILES: CC(=O)Nc1cc2c(cn1)C1(CC1)CN2c1cc(C)cc(OC2CCC(O)C2)n1] LCMS m/z = 395 [M + H]⁺. |
| 338 | N-(1′-(6-((3-methoxycyclopentyl)oxy)-4-methylpyridin-2-yl)-1′,2′- dihydrospiro[cyclopropane-1,3′-pyrrolo[3,2-c]pyridin]-6′-yl)acetamide trifluoroacetate 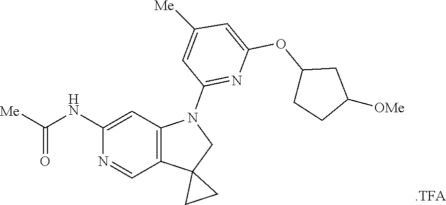 [SMILES: COC1CCC(Oc2cc(C)cc(N3CC4(CC4)c4cnc(NC(C)=O)cc43)n2)C1] LCMS m/z = 409 [M + H]⁺. |
| 339 | N-(1′-(4-methyl-6-((tetrahydrofuran-3-yl)oxy)pyridin-2-yl)-1′,2′- dihydrospiro[cyclopropane-1,3′-pyrrolo[3,2-c]pyridin]-6′-yl)acetamide trifluoroacetate 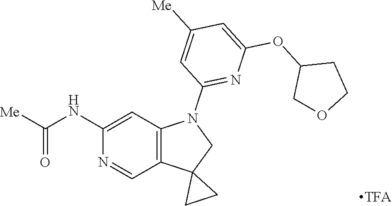 [SMILES: CC(=O)Nc1cc2c(cn1)C1(CC1)CN2c1cc(C)cc(OC2CCOC2)n1] LCMS m/z = 381 [M + H]⁺. |
| 340 | N-(1′-(6-cyclobutoxy-4-methylpyridin-2-yl)-1′,2′-dihydrospiro[cyclopropane- 1,3′-pyrrolo[3,2-c]pyridin]-6′-yl)acetamide trifluroacetate 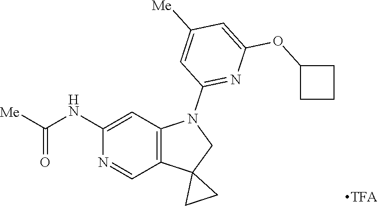 [SMILES: CC(=O)Nc1cc2c(cn1)C1(CC1)CN2c1cc(C)cc(OC2CCC2)n1] LCMS m/z = 365 [M + H]⁺. |
| 341 | N-(1′-(4-methyl-6-(oxetan-3-yloxy)pyridin-2-yl)-1′,2′- dihydrospiro[cyclopropane-1,3′-pyrrolo[3,2-c]pyridin]-6′-yl)acetamide trifluoroacetate 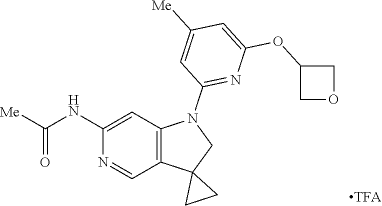 [SMILES: CC(=O)Nc1cc2c(cn1)C1(CC1)CN2c1cc(C)cc(OC2COC2)n1] LCMS m/z = 367 [M + H]⁺. |
| 342 | N-(1′-(6-(3-hydroxycyclobutoxy)-4-methylpyridin-2-yl)-1′,2′- dihydrospiro[cyclopropane-1,3′-pyrrolo[3,2-c]pyridin]-6′-yl)acetamide 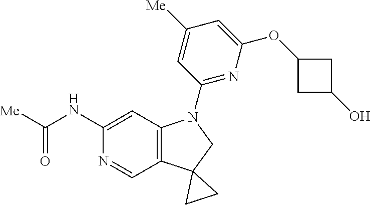 [SMILES: CC(=O)Nc1cc2c(cn1)C1(CC1)CN2c1cc(C)cc(OC2CC(O)C2)n1] LCMS m/z = 381 [M + H]⁺. |
| 343 | N-(1′-(6-(3-methoxycyclobutoxy)-4-methylpyridin-2-yl)-1′,2′- dihydrospiro[cyclopropane-1,3′-pyrrolo[3,2-c]pyridin]-6′-yl)acetamide trifluoroacetate 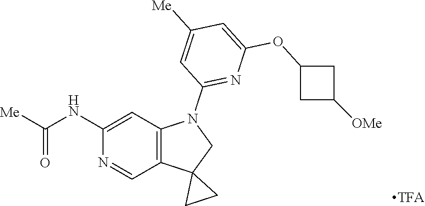 [SMILES: COC1CC(Oc2cc(C)cc(N3CC4(CC4)c4cnc(NC(C)=O)cc43)n2)C1] LCMS m/z = 395 [M + H]⁺. |
| 344 | N-(1′-(6-(3-cyanocyclobutoxy)-4-methylpyridin-2-yl)-1′,2′- dihydrospiro[cyclopropane-1,3′-pyrrolo[3,2-c]pyridin]-6′-yl)acetamide trifluoroacetate 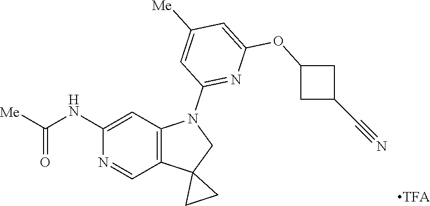 [SMILES: CC(=O)Nc1cc2c(cn1)C1(CC1)CN2c1cc(C)cc(OC2CC(C#N)C2)n1] LCMS m/z = 390 [M + H]⁺. |
| 345 | N-(1′-(6-(3-(methoxymethyl)cyclobutoxy)-4-methylpyridin-2-yl)-1′,2′- dihydrospiro[cyclopropane-1,3′-pyrrolo[3,2-c]pyridin]-6′-yl)acetamide trifluoroacetate 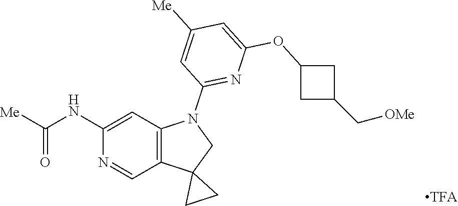 [SMILES: COCC1CC(Oc2cc(C)cc(N3CC4(CC4)c4cnc(NC(C)=O)cc43)n2)C1] LCMS m/z = 409 [M + H]⁺. |
| 346 | N-(1′-(6-(3-fluorocyclobutoxy)-4-methylpyridin-2-yl)-1′,2′- dihydrospiro[cyclopropane-1,3′-pyrrolo[3,2-c]pyridin]-6′-yl)acetamide trifluoroacetate 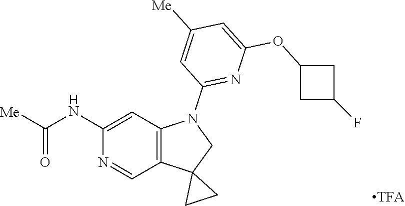 [SMILES: CC(=O)Nc1cc2c(cn1)C1(CC1)CN2c1cc(C)cc(OC2CC(F)C2)n1] LCMS m/z = 383 [M + H]⁺. |
| 347 | N-(1′-(4-methyl-6-(3-methylcyclobutoxy)pyridin-2-yl)-1′,2′- dihydrospiro[cyclopropane-1,3′-pyrrolo[3,2-c]pyridin]-6′-yl)acetamide trifluoroacetate 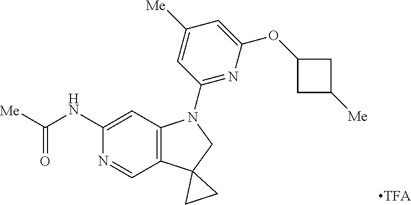 [SMILES: CC(=O)Nc1cc2c(cn1)C1(CC1)CN2c1cc(C)cc(OC2CC(C)C2)n1] LCMS m/z = 379 [M + H]⁺. |
| 348 | N-(1′-(6-(3-fluorocyclobutoxy)-4-methylpyridin-2-yl)-1′,2′- dihydrospiro[cyclopropane-1,3′-pyrrolo[3,2-c]pyridin]-6′-yl)acetamide trifluoroacetate 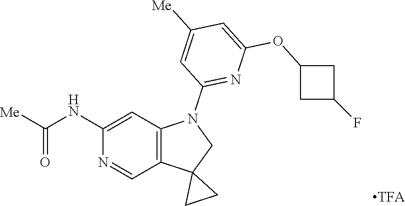 [SMILES: CC(=O)Nc1cc2c(cn1)C1(CC1)CN2c1cc(C)cc(OC2CC(F)C2)n1] LCMS m/z = 383 [M + H]⁺. |
| 349 | N-(1′-(4-methyl-6-((4-methyl-2-oxabicyclo[2.1.1]hexan-1- yl)methoxy)pyridin-2-yl)-1′,2′-dihydrospiro[cyclopropane-1,3′-pyrrolo[3,2- c]pyridin]-6′-yl)acetamide 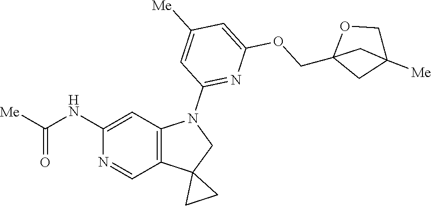 [SMILES: CC(=O)Nc1cc2c(cn1)C1(CC1)CN2c1cc(C)cc(OCC23CC(C)(CO2)C3)n1] LCMS m/z = 421 [M + H]⁺. |
| 350 | N-(1′-(6-((3-methoxycyclobutyl)methoxy)-4-methylpyridin-2-yl)-1′,2′- dihydrospiro[cyclopropane-1,3′-pyrrolo[3,2-c]pyridin]-6′-yl)acetamide 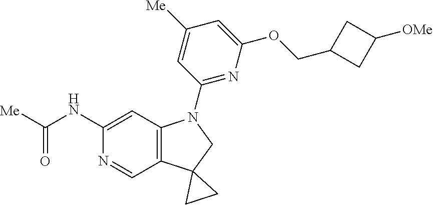 [SMILES: COC1CC(COc2cc(C)cc(N3CC4(CC4)c4cnc(NC(C)=O)cc43)n2)C1] LCMS m/z = 409 [M + H]⁺. |
| 351 | N-(1′-(6-((1-methoxycyclobutyl)methoxy)-4-methylpyridin-2-yl)-1′,2′- dihydrospiro[cyclopropane-1,3′-pyrrolo[3,2-c]pyridin]-6′-yl)acetamide 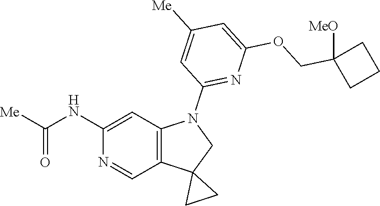 [SMILES: COC1(COc2cc(C)cc(N3CC4(CC4)c4cnc(NC(C)=O)cc43)n2)CCC1] LCMS m/z = 409 [M + H]⁺. |
| 352 | N-(1′-(6-(cyclopropylmethoxy)-4-methylpyridin-2-yl)-1′,2′- dihydrospiro[cyclopropane-1,3′-pyrrolo[3,2-c]pyridin]-6′-yl)acetamide trifluoroacetate 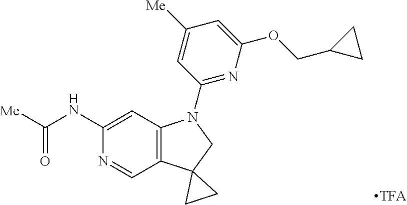 [SMILES: CC(=O)Nc1cc2c(cn1)C1(CC1)CN2c1cc(C)cc(OCC2CC2)n1] LCMS m/z = 365 [M + H]⁺. |
| 353 | N-(1′-(6-((3-fluorocyclopentyl)oxy)-4-methylpyridin-2-yl)-1′,2′- dihydrospiro[cyclopropane-1,3′-pyrrolo[3,2-c]pyridin]-6′-yl)acetamide trifluoroacetate 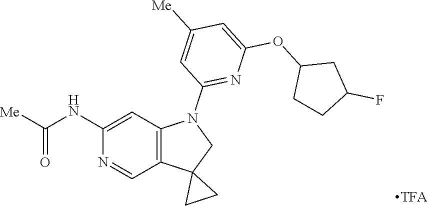 [SMILES: CC(=O)Nc1cc2c(cn1)C1(CC1)CN2c1cc(C)cc(OC2CCC(F)C2)n1] LCMS m/z = 397 [M + H]⁺. |
| 354 | N-(1′-(6-(cyclopentyloxy)-4-methylpyridin-2-yl)-1′,2′- dihydrospiro[cyclopropane-1,3′-pyrrolo[3,2-c]pyridin]-6′-yl)acetamide trifluoroacetate 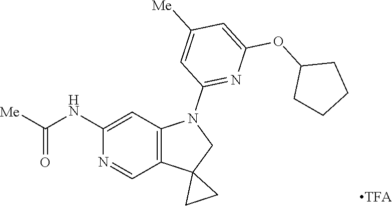 [SMILES: CC(=O)Nc1cc2c(cn1)C1(CC1)CN2c1cc(C)cc(OC2CCCC2)n1] LCMS m/z = 378 [M + H]⁺. |
| 355 | N-(1′-(6-(2-fluoroethoxy)-4-methylpyridin-2-yl)-1′,2′- dihydrospiro[cyclopropane-1,3′-pyrrolo[3,2-c]pyridin]-6′-yl)acetamide trifluoroacetate 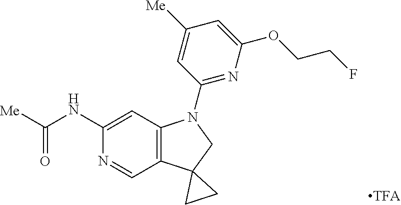 [SMILES: CC(=O)Nc1cc2c(cn1)C1(CC1)CN2c1cc(C)cc(OCCF)n1] LCMS m/z = 357 [M + H]⁺. |
| 356 | N-(1′-(6-(2,2-difluoropropoxy)-4-methylpyridin-2-yl)-1′,2′- dihydrospiro[cyclopropane-1,3′-pyrrolo[3,2-c]pyridin]-6′-yl)acetamide trifluoroacetate 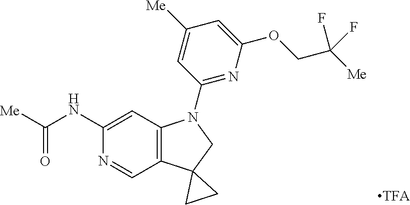 [SMILES: CC(=O)Nc1cc2c(cn1)C1(CC1)CN2c1cc(C)cc(OCC(C)(F)F)n1] LCMS m/z = 389 [M + H]⁺. |
| 357 | N-(1′-(6-(2-methoxyethoxy)-4-methylpyridin-2-yl)-1′,2′- dihydrospiro[cyclopropane-1,3′-pyrrolo[3,2-c]pyridin]-6′-yl)acetamide trifluoroacetate 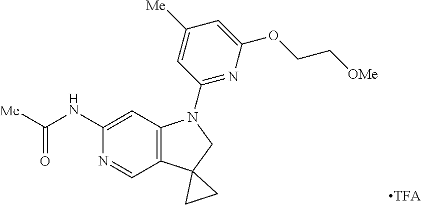 [SMILES: COCCOc1cc(C)cc(N2CC3(CC3)c3cnc(NC(C)=O)cc32)n1] LCMS m/z = 389 [M + H]⁺. |
| 358 | N-(1′-(4-methyl-6-(oxetan-2-ylmethoxy)pyridin-2-yl)-1′,2′- dihydrospiro[cyclopropane-1,3′-pyrrolo[3,2-c]pyridin]-6′-yl)acetamide trifluoroacetate 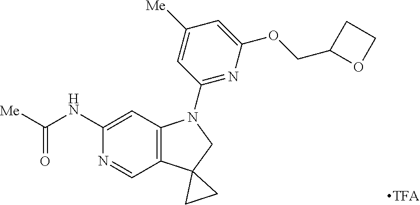 [SMILES: CC(=O)Nc1cc2c(cn1)C1(CC1)CN2c1cc(C)cc(OCC2CCO2)n1] LCMS m/z = 381 [M + H]⁺. |
| 359 | N-(1′-(2-(1,1-difluoroethyl)-6-((1-methylpyrrolidin-3-yl)oxy)pyrimidin-4-yl)- 1′,2′-dihydrospiro[cyclopropane-1,3′-pyrrolo[3,2-c]pyridin]-6′-yl)acetamide trifluoroacetate 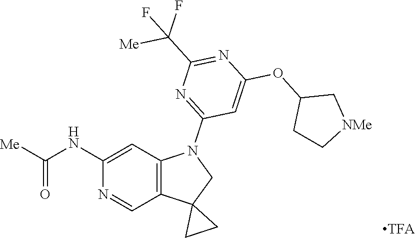 [SMILES: CC(=O)Nc1cc2c(cn1)C1(CC1)CN2c1cc(OC2CCN(C)C2)nc(C(C)(F)F)n1] LCMS m/z = 445 [M + H]⁺. |
| 360 | N-(1′-(2-(2-methoxyethoxy)-6-methylpyrimidin-4-yl)-1′,2′- dihydrospiro[cyclopropane-1,3′-pyrrolo[3,2-c]pyridin]-6′-yl)acetamide trifluoroacetate 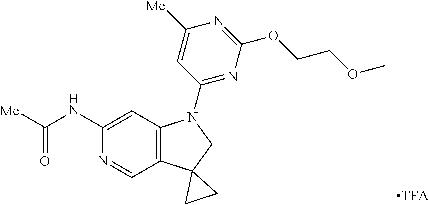 [SMILES: COCCOc1nc(C)cc(N2CC3(CC3)c3cnc(NC(C)=O)cc32)n1] LCMS m/z = 370 [M + H]⁺. |
| 361 | N-(1′-(6-methyl-2-(2,2,2-trifluoroethoxy)pyrimidin-4-yl)-1′,2′- dihydrospiro[cyclopropane-1,3′-pyrrolo[3,2-c]pyridin]-6′-yl)acetamide trifluoroacetate 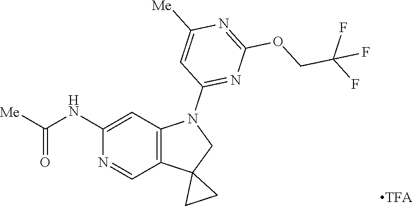 [SMILES: CC(=O)Nc1cc2c(cn1)C1(CC1)CN2c1cc(C)nc(OCC(F)(F)F)n1 ; "·TFA"] LCMS m/z = 394 [M + H]⁺. |
| 362 | N-(1′-(2-isopropoxy-6-methylpyrimidin-4-yl)-1′,2′- dihydrospiro[cyclopropane-1,3′-pyrrolo[3,2-c]pyridin]-6′-yl)acetamide trifluoroacetate 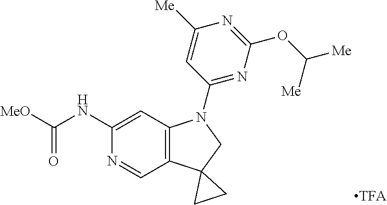 [SMILES: COC(=O)Nc1cc2c(cn1)C1(CC1)CN2c1cc(C)nc(OC(C)C)n1 ; "·TFA"] LCMS m/z = 354 [M + H]⁺. |
| 363 | N-(1′-(2-(3,3-difluorocyclobutoxy)-6-methylpyrimidin-4-yl)-1′,2′- dihydrospiro[cyclopropane-1,3′-pyrrolo[3,2-c]pyridin]-6′-yl)acetamide 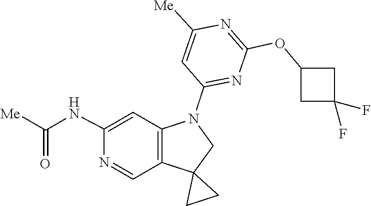 [SMILES: CC(=O)Nc1cc2c(cn1)C1(CC1)CN2c1cc(C)nc(OC2CC(F)(F)C2)n1] LCMS m/z = 402 [M + H]⁺. |
| 364 | N-(1′-(2-((2-oxaspiro[3.3]heptan-6-yl)oxy)-6-methylpyrimidin-4-yl)-1′,2′- dihydrospiro[cyclopropane-1,3′-pyrrolo[3,2-c]pyridin]-6′-yl)acetamide 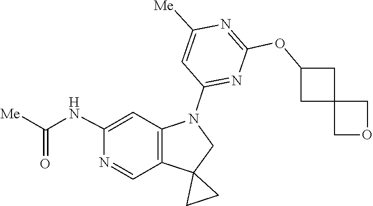 [SMILES: CC(=O)Nc1cc2c(cn1)C1(CC1)CN2c1cc(C)nc(OC2CC3(COC3)C2)n1] LCMS m/z = 408 [M + H]⁺. |
| 365 | N-(1′-(6-methyl-2-((3-methyloxetan-3-yl)oxy)pyrimidin-4-yl)-1′,2′- dihydrospiro[cyclopropane-1,3′-pyrrolo[3,2-c]pyridin]-6′-yl)acetamide 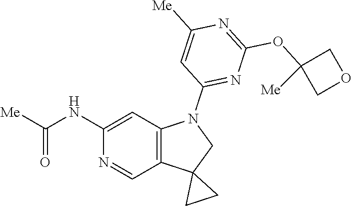 [SMILES: CC(=O)Nc1cc2c(cn1)C1(CC1)CN2c1cc(C)nc(OC2(C)COC2)n1] LCMS m/z = 382 [M + H]⁺. |
| 366 | N-(1′-(6-methyl-2-(oxetan-3-ylmethoxy)pyrimidin-4-yl)-1′,2′- dihydrospiro[cyclopropane-1,3′-pyrrolo[3,2-c]pyridin]-6′-yl)acetamide trifluoroacetate 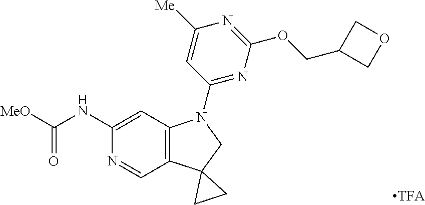 [SMILES: COC(=O)Nc1cc2c(cn1)C1(CC1)CN2c1cc(C)nc(OCC2COC2)n1 ; "·TFA"] LCMS m/z = 382 [M + H]⁺. |
| 367 | N-(1′-(6-methyl-2-(3-methylcyclobutoxy)pyrimidin-4-yl)-1′,2′- dihydrospiro[cyclopropane-1,3′-pyrrolo[3,2-c]pyridin]-6′-yl)acetamide 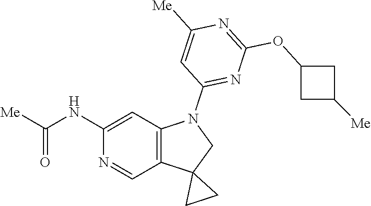 [SMILES: CC(=O)Nc1cc2c(cn1)C1(CC1)CN2c1cc(C)nc(OC2CC(C)C2)n1] LCMS m/z = 380 [M + H]⁺. |
| 368 | N-(1′-(2-(3-cyano-3-fluorocyclobutoxy)-6-methylpyrimidin-4-yl)-1′,2′- dihydrospiro[cyclopropane-1,3′-pyrrolo[3,2-c]pyridin]-6′-yl)acetamide 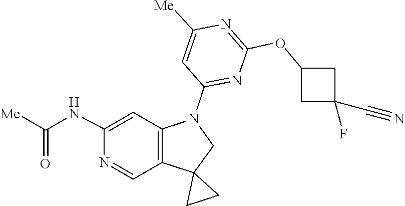 [SMILES: CC(=O)Nc1cc2c(cn1)C1(CC1)CN2c1cc(C)nc(OC2CC(F)(C#N)C2)n1] LCMS m/z = 309 [M + H]⁺. |
| 369 | N-(1′-(2-(cyclopropylmethoxy)-6-methylpyrimidin-4-yl)-1′,2′- dihydrospiro[cyclopropane-1,3′-pyrrolo[3,2-c]pyridin]-6′-yl)acetamide 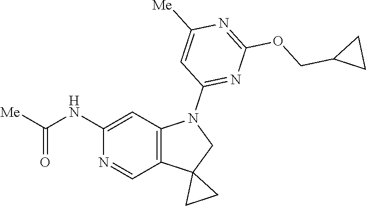 [SMILES: CC(=O)Nc1cc2c(cn1)C1(CC1)CN2c1cc(C)nc(OCC2CC2)n1] LCMS m/z = 366 [M + H]⁺. |
| 370 | N-(1′-(2-(3-fluorocyclobutoxy)-6-methylpyrimidin-4-yl)-1′,2′- dihydrospiro[cyclopropane-1,3′-pyrrolo[3,2-c]pyridin]-6′-yl)acetamide 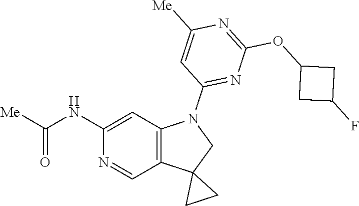 [SMILES: CC(=O)Nc1cc2c(cn1)C1(CC1)CN2c1cc(C)nc(OC2CC(F)C2)n1] LCMS m/z = 384 [M + H]⁺. |
| 371 | N-(1′-(6-methyl-2-((4-methyl-2-oxabicyclo[2.1.1]hexan-1- yl)methoxy)pyrimidin-4-yl)-1′,2′-dihydrospiro[cyclopropane-1,3′-pyrrolo[3,2- c]pyridin]-6′-yl)acetamide 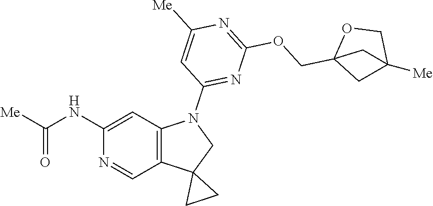 [SMILES: CC(=O)Nc1cc2c(cn1)C1(CC1)CN2c1cc(C)nc(OCC23CC(C)(CO2)C3)n1] LCMS m/z = 422 [M + H]⁺. |
| 372 | N-(1′-(2-((3-methoxycyclobutyl)methoxy)-6-methylpyrimidin-4-yl)-1′,2′- dihydrospiro[cyclopropane-1,3′-pyrrolo[3,2-c]pyridin]-6′-yl)acetamide 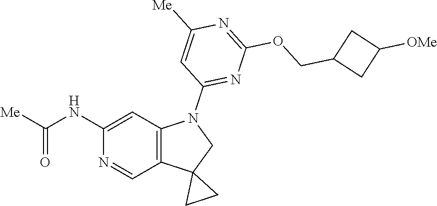 [SMILES: COC1CC(COc2nc(C)cc(N3CC4(CC4)c4cnc(NC(C)=O)cc43)n2)C1] LCMS m/z = 410 [M + H]⁺. |
| 373 | N-(1′-(2-(2-methoxy-2-methylpropoxy)-6-methylpyrimidin-4-yl)-1′,2′- dihydrospiro[cyclopropane-1,3′-pyrrolo[3,2-c]pyridin]-6′-yl)acetamide 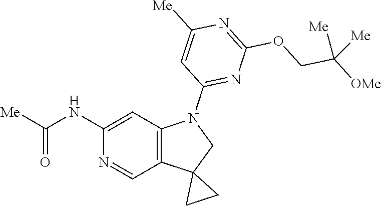 [SMILES: COC(C)(C)COc1nc(C)cc(N2CC3(CC3)c3cnc(NC(C)=O)cc32)n1] LCMS m/z = 398 [M + H]⁺. |
| 374 | N-(1′-(6-methyl-2-((tetrahydrofuran-3-yl)oxy)pyrimidin-4-yl)-1′,2′- dihydrospiro[cyclopropane-1,3′-pyrrolo[3,2-c]pyridin]-6′-yl)acetamide trifluoroacetate 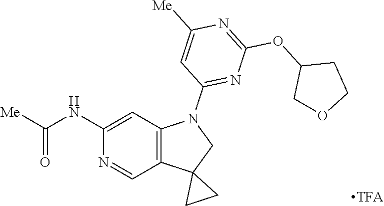 [SMILES: CC(=O)Nc1cc2c(cn1)C1(CC1)CN2c1cc(C)nc(OC2CCOC2)n1 ; "·TFA"] LCMS m/z = 382 [M + H]⁺. |
| 375 | N-(1′-(2-(3-cyanocyclobutoxy)-6-methylpyrimidin-4-yl)-1′,2′- dihydrospiro[cyclopropane-1,3′-pyrrolo[3,2-c]pyridin]-6′-yl)acetamide 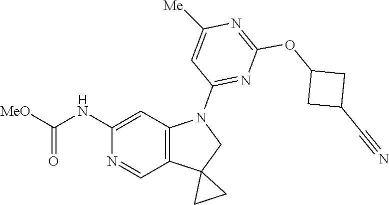 [SMILES: COC(=O)Nc1cc2c(cn1)C1(CC1)CN2c1cc(C)nc(OC2CC(C#N)C2)n1] LCMS m/z = 391 [M + H]⁺. |
| 376 | N-(1′-(2-(3-(methoxymethyl)cyclobutoxy)-6-methylpyrimidin-4-yl)-1′,2′- dihydrospiro[cyclopropane-1,3′-pyrrolo[3,2-c]pyridin]-6′-yl)acetamide 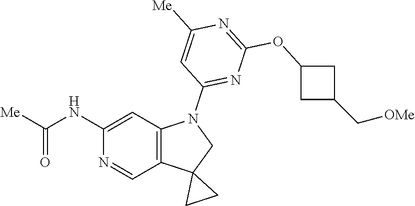 [SMILES: COCC1CC(Oc2nc(C)cc(N3CC4(CC4)c4cnc(NC(C)=O)cc43)n2)C1] LCMS m/z = 410 [M + H]⁺. |
| 377 | N-(1′-(2-(3-fluorocyclobutoxy)-6-methylpyrimidin-4-yl)-1′,2′- dihydrospiro[cyclopropane-1,3′-pyrrolo[3,2-c]pyridin]-6′-yl)acetamide 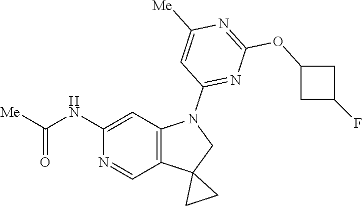 [SMILES: CC(=O)Nc1cc2c(cn1)C1(CC1)CN2c1cc(C)nc(OC2CC(F)C2)n1] LCMS m/z = 394 [M + H]⁺. |
| 378 | N-(1′-(2-((1-methoxycyclobutyl)methoxy)-6-methylpyrimidin-4-yl)-1′,2′- dihydrospiro[cyclopropane-1,3′-pyrrolo[3,2-c]pyridin]-6′-yl)acetamide 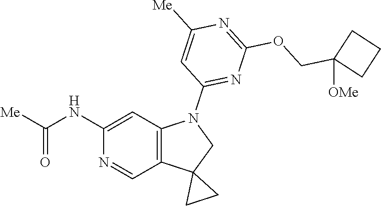 [SMILES: COC1(COc2nc(C)cc(N3CC4(CC4)c4cnc(NC(C)=O)cc43)n2)CCC1] LCMS m/z = 410 [M + H]⁺. |
| 379 | N-(1′-(2-(cyclopentyloxy)-6-methylpyrimidin-4-yl)-1′,2′- dihydrospiro[cyclopropane-1,3′-pyrrolo[3,2-c]pyridin]-6′-yl)acetamide trifluoroacetate 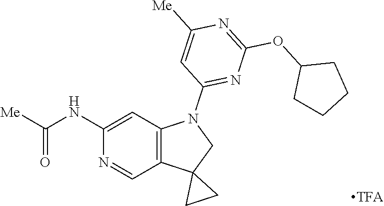 [SMILES: CC(=O)Nc1cc2c(cn1)C1(CC1)CN2c1cc(C)nc(OC2CCCC2)n1 ; "·TFA"] LCMS m/z = 380 [M + H]⁺. |
| 380 | N-(1′-(6-methyl-2-(oxetan-2-ylmethoxy)pyrimidin-4-yl)-1′,2′- dihydrospiro[cyclopropane-1,3′-pyrrolo[3,2-c]pyridin]-6′-yl)acetamide trifluoroacetate 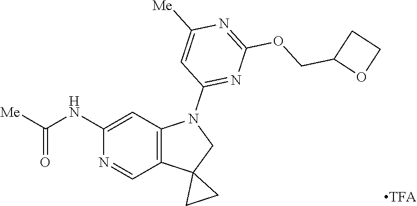 [SMILES: CC(=O)Nc1cc2c(cn1)C1(CC1)CN2c1cc(C)nc(OCC2CCO2)n1 ; "·TFA"] LCMS m/z = 382 [M + H]⁺. |
| 381 | N-(1′-(2-(1,1-difluoroethyl)-6-(3-fluoropyridin-4-yl)pyrimidin-4-yl)-1′,2′- dihydrospiro[cyclopropane-1,3′-pyrrolo[3,2-c]pyridin]-6′-yl)acetamide trifluoroacetate 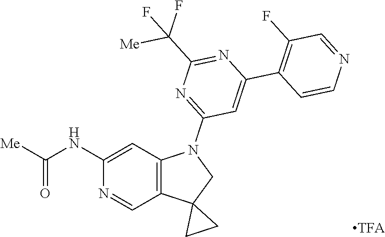 [SMILES: CC(=O)Nc1cc2c(cn1)C1(CC1)CN2c1cc(-c2ccncc2F)nc(C(C)(F)F)n1 ; "·TFA"] |
| 382 | N-(1′-(6-(1-cyclobutyl-1H-pyrazol-4-yl)-2-(1,1-difluoroethyl)pyrimidin-4-yl)- 1′,2′-dihydrospiro[cyclopropane-1,3′-pyrrolo[3,2-c]pyridin]-6′-yl)acetamide trifluoroacetate 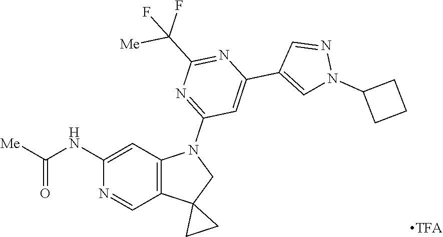 [SMILES: CC(=O)Nc1cc2c(cn1)C1(CC1)CN2c1cc(-c2cnn(C3CCC3)c2)nc(C(C)(F)F)n1 ; "·TFA"] LCMS m/z = 466 [M + H]⁺. |
| 383 | N-(1′-(2-(1,1-difluoroethyl)-6-(1-(difluoromethyl)-1H-pyrazol-4-yl)pyrimidin- 4-yl)-1′,2′-dihydrospiro[cyclopropane-1,3′-pyrrolo[3,2-c]pyridin]-6′- yl)acetamide trifluoroacetate 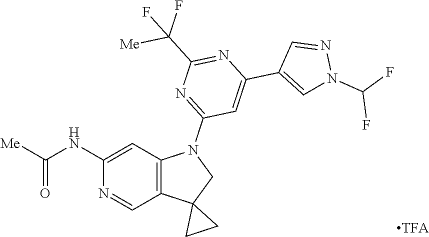 [SMILES: CC(=O)Nc1cc2c(cn1)C1(CC1)CN2c1cc(-c2cnn(C(F)F)c2)nc(C(C)(F)F)n1 ; "·TFA"] LCMS m/z = 462 [M + H]⁺. |
| 384 | N-(1′-(2-(1,1-difluoroethyl)-6-(1-ethyl-1H-pyrazol-4-yl)pyrimidin-4-yl)-1′,2′- dihydrospiro[cyclopropane-1,3′-pyrrolo[3,2-c]pyridin]-6′-yl)acetamide trifluoroacetate 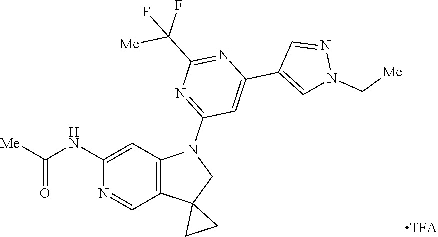 [SMILES: CCn1cc(-c2cc(N3CC4(CC4)c4cnc(NC(C)=O)cc43)nc(C(C)(F)F)n2)cn1 ; "·TFA"] LCMS m/z = 441 [M + H]⁺. |
| 385 | N-(1′-(2-(1,1-difluoroethyl)-6-(5-(difluoromethyl)-1-methyl-1H-pyrazol-4- yl)pyrimidin-4-yl)-1′,2′-dihydrospiro[cyclopropane-1,3′-pyrrolo[3,2- c]pyridin]-6′-yl)acetamide trifluoroacetate 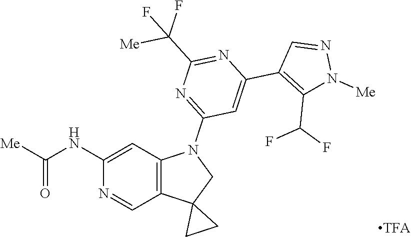 [SMILES: CC(=O)Nc1cc2c(cn1)C1(CC1)CN2c1cc(-c2cnn(C)c2C(F)F)nc(C(C)(F)F)n1 ; "·TFA"] LCMS m/z = 476 [M + H]⁺. |
| 386 | N-(1′-(2-(1,1-difluoroethyl)-6-(1-isopropyl-1H-pyrazol-4-yl)pyrimidin-4-yl)- 1′,2′-dihydrospiro[cyclopropane-1,3′-pyrrolo[3,2-c]pyridin]-6′-yl)acetamide trifluoroacetate 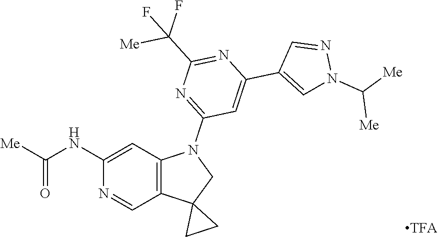 [SMILES: CC(=O)Nc1cc2c(cn1)C1(CC1)CN2c1cc(-c2cnn(C(C)C)c2)nc(C(C)(F)F)n1 ; "·TFA"] LCMS m/z = 455 [M + H]⁺. |
| 387 | N-(1′-(6-(1-cyclopropyl-1H-pyrazol-4-yl)-2-(1,1-difluoroethyl)pyrimidin-4- yl)-1′,2′-dihydrospiro[cyclopropane-1,3′-pyrrolo[3,2-c]pyridin]-6′- yl)acetamide trifluoroacetate 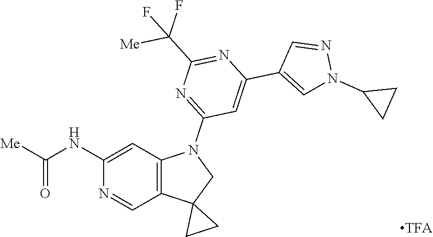 [SMILES: CC(=O)Nc1cc2c(cn1)C1(CC1)CN2c1cc(-c2cnn(C3CC3)c2)nc(C(C)(F)F)n1 ; "·TFA"] LCMS m/z = 452 [M + H]⁺. |
| 388 | N-(1′-(2-(1,1-difluoroethyl)-6-(1-(oxetan-3-yl)-1H-pyrazol-4-yl)pyrimidin-4- yl)-1′,2′-dihydrospiro[cyclopropane-1,3′-pyrrolo[3,2-c]pyridin]-6′- yl)acetamide 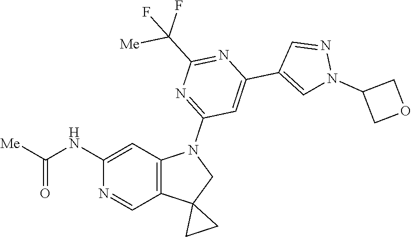 [SMILES: CC(=O)Nc1cc2c(cn1)C1(CC1)CN2c1cc(-c2cnn(C3COC3)c2)nc(C(C)(F)F)n1] LCMS m/z = 468 [M + H]⁺. |
| 389 | N-(1′-(6-(1-(cyclopropylmethyl)-1H-pyrazol-4-yl)-2-(1,1- difluoroethyl)pyrimidin-4-yl)-1′,2′-dihydrospiro[cyclopropane-1,3′- pyrrolo[3,2-c]pyridin]-6′-yl)acetamide 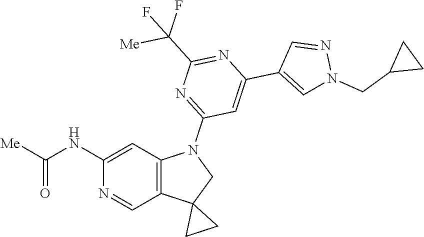 [SMILES: CC(=O)Nc1cc2c(cn1)C1(CC1)CN2c1cc(-c2cnn(CC3CC3)c2)nc(C(C)(F)F)n1] LCMS m/z = 467 [M + H]⁺. |
| 390 | N-(1′-(2-(1,1-difluoroethyl)-6-(1-(2-morpholinoethyl)-1H-pyrazol-4- yl)pyrimidin-4-yl)-1′,2′-dihydrospiro[cyclopropane-1,3′-pyrrolo[3,2- c]pyridin]-6′-yl)acetamide trifluoroacetate 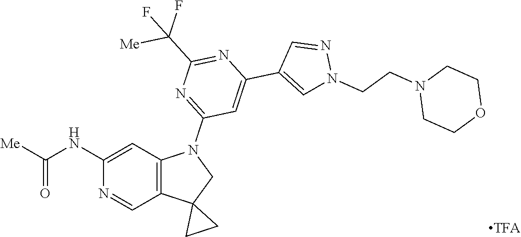 [SMILES: CC(=O)Nc1cc2c(cn1)C1(CC1)CN2c1cc(-c2cnn(CCN3CCOCC3)c2)nc(C(C)(F)F)n1 ; "·TFA"] LCMS m/z = 525 [M + H]⁺. |
| 391 | N-(1′-(2-(1,1-difluoroethyl)-6-(1-(2-(dimethylamino)ethyl)-1H-pyrazol-4- yl)pyrimidin-4-yl)-1′,2′-dihydrospiro[cyclopropane-1,3′-pyrrolo[3,2- c]pyridin]-6′-yl)acetamide trifluoroacetate 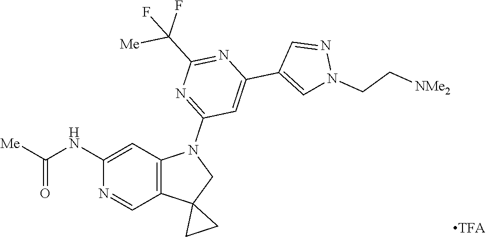 [SMILES: CC(=O)Nc1cc2c(cn1)C1(CC1)CN2c1cc(-c2cnn(CCN(C)C)c2)nc(C(C)(F)F)n1 ; "·TFA"] LCMS m/z = 484 [M + H]⁺. |
| 392 | N-(1′-(2-(1,1-difluoroethyl)-6-(1-(2,2,2-trifluoroethyl)-1H-pyrazol-4- yl)pyrimidin-4-yl)-1′,2′-dihydrospiro[cyclopropane-1,3′-pyrrolo[3,2- c]pyridin]-6′-yl)acetamide 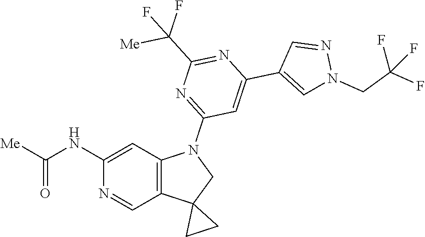 [SMILES: CC(=O)Nc1cc2c(cn1)C1(CC1)CN2c1cc(-c2cnn(CC(F)(F)F)c2)nc(C(C)(F)F)n1] LCMS m/z = 495 [M + H]⁺. |
| 393 | N-(1′-(6-(1-(cyanomethyl)-1H-pyrazol-4-yl)-2-(1,1-difluoroethyl)pyrimidin-4- yl)-1′,2′-dihydrospiro[cyclopropane-1,3′-pyrrolo[3,2-c]pyridin]-6′- yl)acetamide 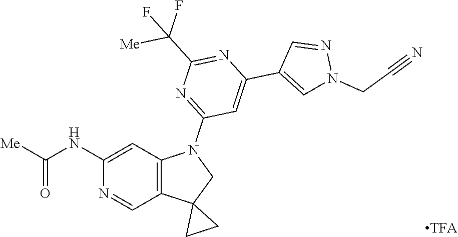 [SMILES: CC(=O)Nc1cc2c(cn1)C1(CC1)CN2c1cc(-c2cnn(CC#N)c2)nc(C(C)(F)F)n1 ; "·TFA"] LCMS m/z = 451 [M + H]⁺. |
| 394 | N-(1′-(2-(1,1-difluoroethyl)-6-(1-(2-methoxyethyl)-1H-pyrazol-4- yl)pyrimidin-4-yl)-1′,2′-dihydrospiro[cyclopropane-1,3′-pyrrolo[3,2- c]pyridin]-6′-yl)acetamide 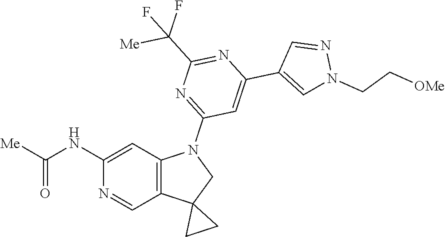 [SMILES: COCCn1cc(-c2cc(N3CC4(CC4)c4cnc(NC(C)=O)cc43)nc(C(C)(F)F)n2)cn1] LCMS m/z = 471 [M + H]⁺. |
| 395 | N-(1′-(2-(1,1-difluoroethyl)-6-(1H-pyrazol-4-yl)pyrimidin-4-yl)-1′,2′- dihydrospiro[cyclopropane-1,3′-pyrrolo[3,2-c]pyridin]-6′-yl)acetamide trifluoroacetate 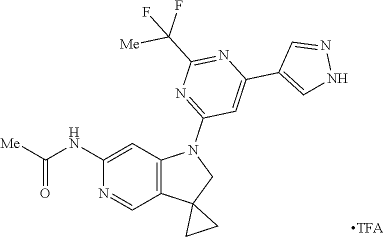 [SMILES: CC(=O)Nc1cc2c(cn1)C1(CC1)CN2c1cc(-c2cn[nH]c2)nc(C(C)(F)F)n1 ; "·TFA"] LCMS m/z = 412 [M + H]⁺. |
| 396 | N-(1′-(6-methyl-2-(oxetan-3-yl)pyrimidin-4-yl)-1′,2′- dihydrospiro[cyclopropane-1,3′-pyrrolo[3,2-c]pyridin]-6′-yl)acetamide 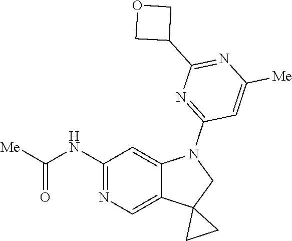 [SMILES: CC(=O)Nc1cc2c(cn1)C1(CC1)CN2c1cc(C)nc(C2COC2)n1] LCMS m/z = 329 [M + H]⁺; ¹H NMR (500 MHz, CDCl₃) δ 9.2-9.1 (m, 1H), 8.8-8.4 (m, 1H), 7.5-7.4 (m, 1H), 6.5-6.4 (m, 1H), 5.2-5.1 (m, 4H), 4.6-4.4 (m, 1H), 4.15 (s, 2H), 2.51 (s, 3H), 2.3-2.2 (m, 3H), 1.2-1.1 (m, 4H). |
| 397 | N-(1′-(2-(1,1-difluoroethyl)-6-(methoxymethyl)pyrimidin-4-yl)-1′,2′- dihydrospiro[cyclopropane-1,3′-pyrrolo[3,2-c]pyridin]-6′-yl)acetamide 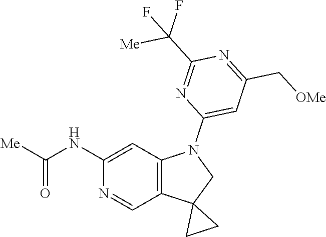 [SMILES: COCc1cc(N2CC3(CC3)c3cnc(NC(C)=O)cc32)nc(C(C)(F)F)n1] LCMS m/z = 390 [M + H]⁺; ¹H NMR (400 MHz, CDCl₃) δ 9.50-9.20 (m, 1H), 7.40 (s, 1H), 6.91 (s, 1H), 4.61 (s, 2H), 4.23 (s, 2H), 3.55 (s, 3H), 2.28 (s, 3H), 2.22 (t, 3H, J = 18.9 Hz), 1.30-1.20 (m, 4H). |
| 398 | N-(1′-(2-(1,1-difluoroethyl)-6-(2-fluoropropan-2-yl)pyrimidin-4-yl)-1′,2′- dihydrospiro[cyclopropane-1,3′-pyrrolo[3,2-c]pyridin]-6′-yl)acetamide 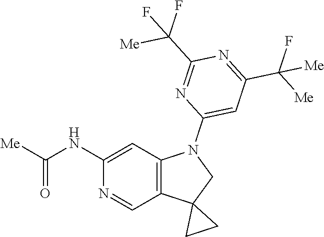 [SMILES: CC(=O)Nc1cc2c(cn1)C1(CC1)CN2c1cc(C(C)(C)F)nc(C(C)(F)F)n1] LCMS m/z = 406 [M + H]⁺; ¹H NMR (400 MHz, CDCl₃) δ 9.25 (br s, 1H), 9.1- 8.7 (m, 1H), 7.46 (s, 1H), 6.92 (s, 1H), 4.20 (s, 2H), 2.3-2.1 (m, 6H), 1.8-1.7 (m, 6H), 1.23 (s, 4H). |
| 399 | N-(1′-(2-(1,1-difluoroethyl)-6-(2-hydroxypropan-2-yl)pyrimidin-4-yl)-1′,2′- dihydrospiro[cyclopropane-1,3′-pyrrolo[3,2-c]pyridin]-6′-yl)acetamide 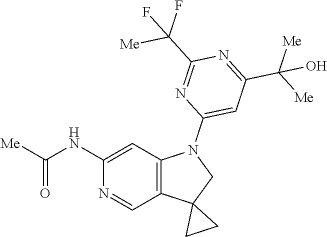 [SMILES: CC(=O)Nc1cc2c(cn1)C1(CC1)CN2c1cc(C(C)(C)O)nc(C(C)(F)F)n1] LCMS m/z = 404 [M + H]⁺; ¹H NMR (400 MHz, DMSO-d₆) δ 10.31 (s, 1H), 9.03 (s, 1H), 7.70 (s, 1H), 7.08 (s, 1H), 5.46 (s, 1H), 4.19 (s, 2H), 2.17 (t, 3H, J = 19.3 Hz), 2.07 (s, 3H), 1.44 (s, 6H), 1.3-1.1 (m, 4H). |
| 400 | N-(1′-(2-(1,1-difluoroethyl)-6-(1-fluoroethyl)pyrimidin-4-yl)-1′,2′- dihydrospiro[cyclopropane-1,3′-pyrrolo[3,2-c]pyridin]-6′-yl)acetamide 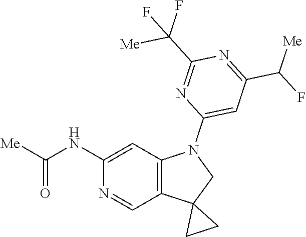 [SMILES: CC(=O)Nc1cc2c(cn1)C1(CC1)CN2c1cc(C(C)F)nc(C(C)(F)F)n1] LCMS m/z = 392 [M + H]⁺; ¹H NMR (400 MHz, CDCl₃) δ 9.3-9.1 (m, 1H), 8.8-8.4 (m, 1H), 7.49 (s, 1H), 6.85 (s, 1H), 5.8-5.6 (m, 1H), 4.19 (s, 2H), 2.3- 2.2 (m, 6H), 1.8-1.7 (m, 3H), 1.23 (s, 4H). |
| 401 | N-(1′-(2-(1,1-difluoro-2-methoxyethyl)-6-methylpyrimidin-4-yl)-1′,2′- dihydrospiro[cyclopropane-1,3′-pyrrolo[3,2-c]pyridin]-6′-yl)acetamide trifluoroacetate 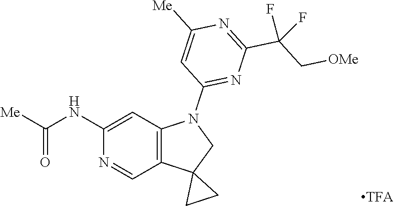 [SMILES: COCC(F)(F)c1nc(C)cc(N2CC3(CC3)c3cnc(NC(C)=O)cc32)n1 ; "·TFA"] LCMS m/z = 390.1 [M + H]⁺. ¹H NMR (600 MHz, DMSO-d₆) δ ppm 10.40 (br s, 1H), 9.02 (br s, 1H), 7.74 (s, 1H), 6.86 (s, 1H), 4.36 (t, J = 13.73 Hz, 2H), 4.19 (s, 2H), 3.41 (s, 3H), 2.50 (s, 3H), 2.12 (s, 3H), 1.17-1.25 (m, 4H). |
| 402 | (S)-N-(1′-(6-(3-cyanopyrrolidin-1-yl)-4-methylpyridin-2-yl)-1′,2′- dihydrospiro[cyclopropane-1,3′-pyrrolo[3,2-c]pyridin]-6′-yl)acetamide 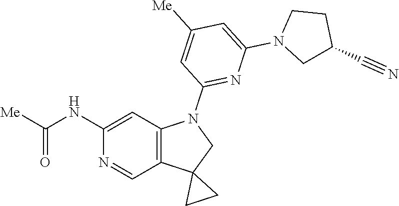 [SMILES: CC(=O)Nc1cc2c(cn1)C1(CC1)CN2c1cc(C)cc(N2CC[C@H](C#N)C2)n1] LCMS m/z = 389 [M + H]⁺. |
| 403 | (R)-N-(1′-(6-(3-cyanopyrrolidin-1-yl)-4-methylpyridin-2-yl)-1′,2′- dihydrospiro[cyclopropane-1,3′-pyrrolo[3,2-c]pyridin]-6′-yl)acetamide 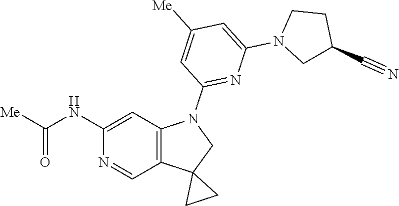 [SMILES: CC(=O)Nc1cc2c(cn1)C1(CC1)CN2c1cc(C)cc(N2CC[C@@H](C#N)C2)n1] LCMS m/z = 389 [M + H]⁺. |
| 404 | N-(1′-(6-(3-cyano-3-methylpyrrolidin-1-yl)-4-methylpyridin-2-yl)-1′,2′- dihydrospiro[cyclopropane-1,3′-pyrrolo[3,2-c]pyridin]-6′-yl)acetamide 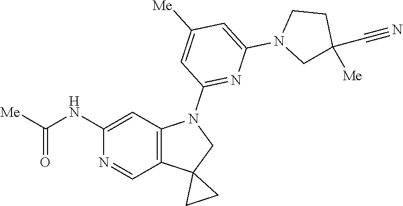 [SMILES: CC(=O)Nc1cc2c(cn1)C1(CC1)CN2c1cc(C)cc(N2CCC(C)(C#N)C2)n1] LCMS m/z = 403 [M + H]⁺. |
| 405 | N-(1′-(6-(3-hydroxy-3-methylpyrrolidin-1-yl)-4-methylpyridin-2-yl)-1′,2′- dihydrospiro[cyclopropane-1,3′-pyrrolo[3,2-c]pyridin]-6′-yl)acetamide trifluoroacetate 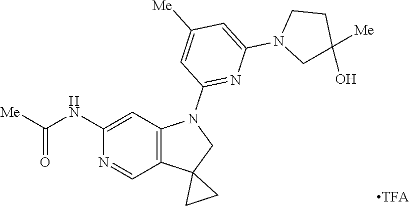 [SMILES: CC(=O)Nc1cc2c(cn1)C1(CC1)CN2c1cc(C)cc(N2CCC(C)(O)C2)n1 ; "·TFA"] LCMS m/z = 394 [M + H]⁺. |
| 406 | N-(1′-(6-(4-(2-hydroxyethyl)piperazin-1-yl)-4-methylpyridin-2-yl)-1′,2′- dihydrospiro[cyclopropane-1,3′-pyrrolo[3,2-c]pyridin]-6′-yl)acetamide 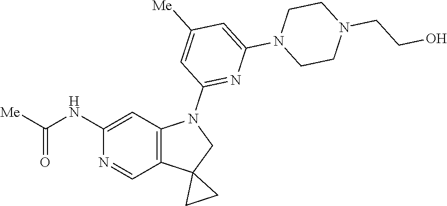 [SMILES: CC(=O)Nc1cc2c(cn1)C1(CC1)CN2c1cc(C)cc(N2CCN(CCO)CC2)n1] LCMS m/z = 423 [M + H]⁺. |
| 407 | (R)-N-(1′-(2-(3-cyanopyrrolidin-1-yl)-6-methylpyrimidin-4-yl)-1′,2′- dihydrospiro[cyclopropane-1,3′-pyrrolo[3,2-c]pyridin]-6′-yl)acetamide 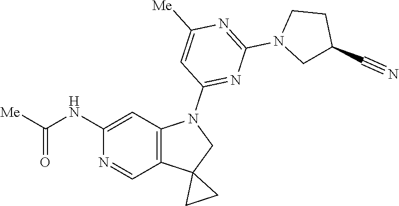 [SMILES: CC(=O)Nc1cc2c(cn1)C1(CC1)CN2c1cc(C)nc(N2CC[C@@H](C#N)C2)n1] LCMS m/z = 390 [M + H]⁺. |
| 408 | (S)-N-(1′-(2-(3-cyanopyrrolidin-1-yl)-6-methylpyrimidin-4-yl)-1′,2′- dihydrospiro[cyclopropane-1,3′-pyrrolo[3,2-c]pyridin]-6′-yl)acetamide 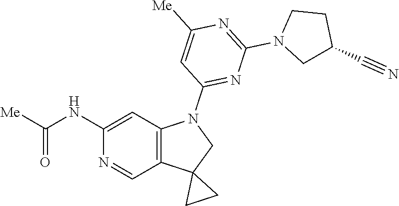 [SMILES: CC(=O)Nc1cc2c(cn1)C1(CC1)CN2c1cc(C)nc(N2CC[C@H](C#N)C2)n1] LCMS m/z = 390 [M + H]⁺. |
| 409 | N-(1′-(2-(3-cyano-3-methylpyrrolidin-1-yl)-6-methylpyrimidin-4-yl)-1′,2′- dihydrospiro[cyclopropane-1,3′-pyrrolo[3,2-c]pyridin]-6′-yl)acetamide 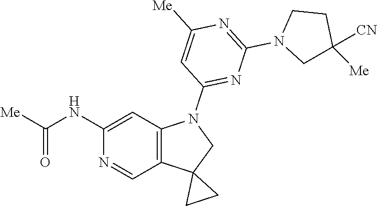 [SMILES: CC(=O)Nc1cc2c(cn1)C1(CC1)CN2c1cc(C)nc(N2CCC(C)(C#N)C2)n1] LCMS m/z = 404 [M + H]⁺. |
| 410 | N-(1′-(2-(3-isobutylazetidin-1-yl)-6-methylpyrimidin-4-yl)-1′,2′- dihydrospiro[cyclopropane-1,3′-pyrrolo[3,2-c]pyridin]-6′-yl)acetamide trifluoroacetate 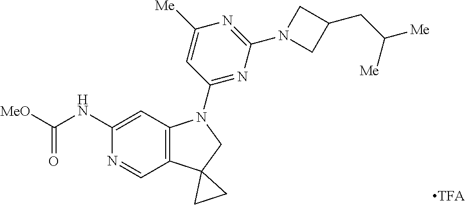 [SMILES: COC(=O)Nc1cc2c(cn1)C1(CC1)CN2c1cc(C)nc(N2CC(CC(C)C)C2)n1 ; "·TFA"] LCMS m/z = 407 [M + H]⁺. |
| 411 | N-(1′-(6-methyl-2-((3aR,6aS)-5-methylhexahydropyrrolo[3,4-c]pyrrol-2(1H)- yl)pyrimidin-4-yl)-1′,2′-dihydrospiro[cyclopropane-1,3′-pyrrolo[3,2- c]pyridin]-6′-yl)acetamide 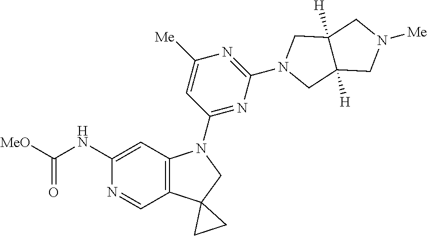 [SMILES: COC(=O)Nc1cc2c(cn1)C1(CC1)CN2c1cc(C)nc(N2C[C@H]3CN(C)C[C@H]3C2)n1] LCMS m/z = 419 [M + H]⁺. |

### Biological Assays

Compounds of the disclosure were assessed for their ability to inhibit TYK2, JAK1 and JAK2 activity. The inhibitory properties of the compounds of the disclosure described herein can be evidenced by testing in any one of the following protocols.

### JH12 Biochemical Assay

The inhibitory potency of compounds of the disclosure against the kinase activity of recombinantly generated JH2 domain of human Tyk2 was evaluated in a plate-based assay using a TR-FRET assay platform. Briefly, 2 nM of recombinant JH2 domain [10×His-tagged TYK2 JH2 domain (amino acid 575-876)] was combined with 2 nM probe ((S)-6-amino-9-(2-carboxy-4-((1-(3-(8-methyl-5-(methylamino)-8H-imidazo[4,5-d]thiazolo[5,4-b]pyridin-2-yl)phenyl)ethyl)carbamoyl)phenyl)-3-iminio-5-sulfo-3H-xanthene-4-sulfonate), 0.1 nM Tb-labeled anti-His antibody, and compounds of disclosure for 60 minutes. Compounds are tested at either 10 M or 1 M top concentration, 10 points of 3-fold dilution. The TR-FRET signal inversely correlates to the amount of probe displaced by compounds and signal was calculated by taking the ratio of fluorescence at 520 nm and 495 nm. The data was normalized and the percent activity versus log concentration of compound was fitted with a 4-parameter logistic model to generate IC₅₀ curves.

pSTAT4 Cell Assay

The inhibitory potency of compounds of the disclosure against the Tyk2 kinase activity on STAT4 was evaluated using an MSD-platform plate-based assay format. NK92 cells natively expressing STAT4 and Tyk2 were serum-starved to reduce background phosphorylation levels, then cells were treated compounds for 1 hr with a 10-point four-fold dilution series starting at 10 μM. Cells were then stimulated with 30 ng/mL IL2 for 15 minutes. Cells were lysed and pSTAT5 levels were quantitated using an MSD plate-based assay with anti-STAT4 antibodies. The data were normalized and the percent activity versus log concentration of compound were fitted with a 4-parameter logistic model to generate to generate IC₅₀ curves.

pSTAT5 Cell Assay

Compounds of the disclosure were assessed for their ability to inhibit the JAK2 kinase activity on STAT5 utilizing an MSD-platform plate-based assay format. TF1 cells natively expressing STAT5 and JAK2 were serum-starved to reduce background phosphorylation levels, then cells were treated with compounds of disclosure for 1 hour with a 10-point four-fold dilution series starting at 10 μM. Cells were then stimulated with 30 ng/mL IL-3 for 15 minutes. Cells were then lysed and pSTAT5 levels were quantitated using an MSD plate-based assay with anti-STAT5 antibodies. The data were normalized and the percent activity versus log concentration of compound was fitted with a 4-parameter logistic model to generate a curve and an IC₅₀ value.

pSTAT3 Cell Assay

The inhibitory potency of compounds of the disclosure against the JAK1 kinase activity on STAT3 was evaluated using an MSD-platform plate-based assay format. TF1 cells natively expressing STAT3 and JAK1 were serum-starved to reduce background phosphorylation levels, then cells were treated with compounds of the disclosure for 1 hour with a 10-point four-fold dilution series starting at 10 μM. Cells were then stimulated with 30 ng/mL interleukin 6 (IL-6) for 15 minutes. Cells were lysed and pSTAT3 levels were quantitated using an MSD plate-based assay with anti-STAT3 antibodies. The data were normalized and the percent activity versus log concentration of compound was fitted with a 4-parameter logistic model to generate IC₅₀ curves.

### Data for Examples

Table 1 shows the inhibitory activity of selected compounds of this disclosure to assess their ability to inhibit TYK2, JAK1 and JAK2, wherein each compound number corresponds to the compound numbering set forth in Examples 1-411 described herein. The measured IC₅₀ values were scored according to the following hierarchy:
“++++” represents: IC₅₀≤10 nM
“+++” represents: 10 nM<IC₅₀≤100 nM
“++” represents: 100 nM<IC₅₀<1000 nM
“+” represents: 1000 nM≤IC₅₀

TABLE 1
Experimental Data

| | | TYK2; | JAK2; | JAK1; |
|---|---|---|---|---|
| | TYK2-JH2 | pSTAT4 | pSTAT5 | pSTAT3 |
| Ex. No. | IC₅₀ (nM) | IC₅₀ (nM) | IC₅₀ (nM) | IC₅₀ (nM) |
| 1 | ++++ | ++ | + | + |
| 2 | ++++ | ++ | + | + |
| 3 | +++ | NT | NT | NT |
| 4 | ++++ | ++ | NT | NT |
| 5 | ++++ | +++ | NT | NT |
| 6 | ++++ | +++ | NT | NT |
| 7 | ++++ | ++ | NT | NT |
| 8 | ++++ | ++ | NT | NT |
| 9 | ++++ | +++ | + | + |
| 10 | ++++ | +++ | + | + |
| 11 | ++++ | +++ | + | + |
| 12 | ++++ | +++ | + | + |
| 13 | +++ | ++ | + | + |
| 14 | +++ | ++ | + | + |
| 15 | ++++ | NT | NT | NT |
| 16 | ++++ | +++ | + | + |
| 17 | ++++ | ++ | + | + |
| 18 | ++++ | +++ | + | + |
| 19 | ++++ | +++ | + | + |
| 20 | ++++ | ++ | NT | NT |
| 21 | ++++ | +++ | + | + |
| 22 | ++++ | ++ | NT | NT |
| 23 | ++++ | +++ | NT | NT |
| 24 | ++++ | +++ | NT | NT |
| 25 | ++++ | ++ | NT | NT |
| 26 | ++++ | ++ | NT | NT |
| 27 | +++ | NT | NT | NT |
| 28 | + | NT | NT | NT |
| 29 | ++++ | ++ | NT | NT |
| 30 | ++ | NT | NT | NT |
| 31 | ++ | NT | NT | NT |
| 32 | +++ | + | + | + |
| 33 | +++ | NT | NT | NT |
| 34 | +++ | + | NT | NT |
| 35 | +++ | + | NT | NT |
| 36 | +++ | NT | NT | NT |
| 37 | +++ | + | + | + |
| 38 | + | + | + | + |
| 39 | + | + | + | + |
| 40 | +++ | NT | NT | NT |
| 41 | +++ | + | + | + |
| 42 | +++ | + | + | + |
| 43 | ++ | NT | NT | NT |
| 44 | ++ | NT | NT | NT |
| 45 | ++ | NT | NT | NT |
| 46 | + | NT | NT | NT |
| 47 | + | NT | NT | NT |
| 48 | ++ | NT | NT | NT |
| 49 | ++++ | +++ | + | + |
| 50 | ++++ | ++ | NT | NT |
| 51 | ++++ | ++ | NT | NT |
| 52 | ++++ | ++ | NT | NT |
| 53 | ++++ | +++ | NT | NT |
| 54 | ++++ | ++ | NT | NT |
| 55 | ++++ | ++ | NT | NT |
| 56 | +++ | + | NT | NT |
| 57 | NT | + | NT | NT |
| 58 | +++ | + | NT | NT |
| 59 | ++ | + | NT | NT |
| 60 | ++++ | ++ | NT | NT |
| 61 | +++ | + | NT | NT |
| 62 | ++++ | ++ | NT | NT |
| 63 | +++ | + | NT | NT |
| 64 | +++ | + | NT | NT |
| 65 | +++ | ++ | NT | NT |
| 66 | ++++ | ++ | NT | NT |
| 67 | ++ | + | NT | NT |
| 68 | +++ | NT | NT | NT |
| 69 | ++++ | ++ | NT | NT |
| 70 | ++++ | ++ | NT | NT |
| 71 | +++ | + | NT | NT |
| 72 | ++++ | ++ | NT | NT |
| 73 | ++++ | +++ | NT | NT |
| 74 | ++++ | +++ | NT | NT |
| 75 | ++++ | ++ | NT | NT |
| 76 | ++ | + | + | + |
| 77 | ++ | NT | NT | NT |
| 78 | +++ | NT | NT | NT |
| 79 | +++ | + | NT | NT |
| 80 | +++ | + | NT | NT |
| 81 | +++ | + | NT | NT |
| 82 | ++++ | ++ | NT | NT |
| 83 | ++++ | ++ | + | + |
| 84 | +++ | + | + | + |
| 85 | +++ | + | + | + |
| 86 | ++++ | ++++ | NT | NT |
| 87 | +++ | + | + | + |
| 88 | ++++ | +++ | + | + |
| 89 | ++++ | ++ | NT | NT |
| 90 | ++++ | +++ | + | + |
| 91 | +++ | + | NT | NT |
| 92 | NT | +++ | NT | NT |
| 93 | ++++ | ++++ | NT | NT |
| 94 | ++ | + | NT | NT |
| 95 | ++++ | ++ | NT | NT |
| 96 | NT | +++ | NT | NT |
| 97 | ++++ | ++ | NT | NT |
| 98 | +++ | + | NT | NT |
| 99 | +++ | + | NT | NT |
| 100 | +++ | + | NT | NT |
| 101 | ++++ | ++ | NT | NT |
| 102 | +++ | NT | NT | NT |
| 103 | ++++ | ++ | NT | NT |
| 104 | ++++ | ++ | NT | NT |
| 105 | NT | + | NT | NT |
| 106 | NT | + | NT | NT |
| 107 | NT | + | NT | NT |
| 108 | NT | + | NT | NT |
| 109 | NT | ++ | NT | NT |
| 110 | NT | + | NT | NT |
| 111 | NT | + | NT | NT |
| 112 | NT | + | NT | NT |
| 113 | +++ | + | NT | NT |
| 114 | +++ | ++ | NT | NT |
| 115 | NT | + | NT | NT |
| 116 | +++ | + | NT | NT |
| 117 | + | NT | NT | NT |
| 118 | + | NT | NT | NT |
| 119 | +++ | ++ | NT | NT |
| 120 | ++ | + | NT | NT |
| 121 | + | NT | NT | NT |
| 122 | ++ | + | NT | NT |
| 123 | ++++ | ++ | NT | NT |
| 124 | NT | +++ | NT | NT |
| 125 | ++++ | ++ | NT | NT |
| 126 | +++ | + | NT | NT |
| 127 | ++++ | ++ | NT | NT |
| 128 | +++ | + | NT | NT |
| 129 | +++ | ++ | NT | NT |
| 130 | ++++ | ++ | NT | NT |
| 131 | +++ | NT | NT | NT |
| 132 | +++ | NT | NT | NT |
| 133 | ++++ | + | NT | NT |
| 134 | + | NT | NT | NT |
| 135 | + | NT | NT | NT |
| 136 | ++++ | +++ | NT | NT |
| 137 | ++++ | ++ | NT | NT |
| 138 | +++ | + | NT | NT |
| 139 | NT | + | NT | NT |
| 140 | +++ | + | NT | NT |
| 141 | ++++ | ++ | NT | NT |
| 142 | +++ | + | NT | NT |
| 143 | ++++ | ++ | NT | NT |
| 144 | +++ | + | NT | NT |
| 145 | +++ | ++ | NT | NT |
| 146 | NT | + | NT | NT |
| 147 | NT | + | NT | NT |
| 148 | + | NT | NT | NT |
| 149 | +++ | + | NT | NT |
| 150 | +++ | NT | NT | NT |
| 151 | +++ | + | NT | NT |
| 152 | +++ | ++ | NT | NT |
| 153 | ++++ | +++ | NT | NT |
| 154 | ++++ | ++++ | NT | NT |
| 155 | ++++ | +++ | NT | NT |
| 156 | ++++ | ++++ | NT | NT |
| 157 | ++++ | ++++ | NT | NT |
| 158 | ++++ | ++++ | NT | NT |
| 159 | NT | +++ | NT | NT |
| 160 | ++++ | +++ | NT | NT |
| 161 | ++++ | +++ | NT | NT |
| 162 | ++++ | +++ | NT | NT |
| 163 | ++++ | +++ | NT | NT |
| 164 | ++++ | +++ | NT | NT |
| 165 | ++++ | +++ | NT | NT |
| 166 | ++++ | +++ | NT | NT |
| 167 | ++++ | ++ | NT | NT |
| 168 | +++ | ++ | NT | NT |
| 169 | ++++ | +++ | NT | NT |
| 170 | ++++ | +++ | NT | NT |
| 171 | ++++ | +++ | NT | NT |
| 172 | ++++ | +++ | NT | NT |
| 173 | ++++ | +++ | NT | NT |
| 174 | +++ | + | NT | NT |
| 175 | ++++ | ++ | NT | NT |
| 176 | ++++ | +++ | NT | NT |
| 177 | ++++ | +++ | NT | NT |
| 178 | ++++ | +++ | NT | NT |
| 179 | ++++ | +++ | NT | NT |
| 180 | +++ | ++ | NT | NT |
| 181 | ++++ | +++ | NT | NT |
| 182 | ++++ | +++ | NT | NT |
| 183 | ++ | + | NT | NT |
| 184 | +++ | ++ | NT | NT |
| 185 | ++ | + | NT | NT |
| 186 | ++ | + | NT | NT |
| 187 | ++ | + | NT | NT |
| 188 | ++ | + | NT | NT |
| 189 | +++ | + | NT | NT |
| 190 | +++ | + | NT | NT |
| 191 | ++ | + | NT | NT |
| 192 | ++ | + | NT | NT |
| 193 | +++ | + | NT | NT |
| 194 | ++++ | + | NT | NT |
| 195 | +++ | + | NT | NT |
| 196 | +++ | ++ | NT | NT |
| 197 | +++ | + | NT | NT |
| 198 | ++ | + | NT | NT |
| 199 | +++ | + | NT | NT |
| 200 | +++ | ++ | NT | NT |
| 201 | +++ | + | NT | NT |
| 202 | +++ | + | NT | NT |
| 203 | ++ | + | NT | NT |
| 204 | ++ | + | NT | NT |
| 205 | +++ | + | NT | NT |
| 206 | +++ | ++ | NT | NT |
| 207 | ++ | + | NT | NT |
| 208 | ++ | + | NT | NT |
| 209 | ++ | + | NT | NT |
| 210 | ++ | + | NT | NT |
| 211 | ++++ | + | NT | NT |
| 212 | ++ | + | NT | NT |
| 213 | +++ | + | NT | NT |
| 214 | ++ | + | NT | NT |
| 215 | NT | + | NT | NT |
| 216 | +++ | ++ | NT | NT |
| 217 | ++++ | ++ | NT | NT |
| 218 | ++++ | NT | NT | NT |
| 219 | +++ | ++ | NT | NT |
| 220 | +++ | + | NT | NT |
| 221 | ++++ | ++ | NT | NT |
| 222 | +++ | + | NT | NT |
| 223 | +++ | + | NT | NT |
| 224 | ++ | + | NT | NT |
| 225 | ++ | + | NT | NT |
| 226 | +++ | + | NT | NT |
| 227 | ++ | + | NT | NT |
| 228 | ++ | + | NT | NT |
| 229 | ++++ | ++ | NT | NT |
| 230 | ++ | NT | NT | NT |
| 231 | ++ | + | NT | NT |
| 232 | ++ | + | NT | NT |
| 233 | + | + | NT | NT |
| 234 | +++ | ++ | NT | NT |
| 235 | ++++ | ++ | NT | NT |
| 236 | +++ | + | NT | NT |
| 237 | +++ | + | NT | NT |
| 238 | ++++ | ++++ | + | + |
| 239 | ++++ | +++ | NT | NT |
| 240 | ++++ | +++ | NT | NT |
| 241 | ++++ | +++ | NT | NT |
| 242 | ++++ | +++ | NT | NT |
| 243 | ++++ | +++ | NT | NT |
| 244 | +++ | + | NT | NT |
| 245 | +++ | ++ | NT | NT |
| 246 | NT | + | NT | NT |
| 247 | ++++ | ++ | NT | NT |
| 248 | +++ | + | NT | NT |
| 249 | +++ | + | NT | NT |
| 250 | +++ | + | NT | NT |
| 251 | +++ | + | NT | NT |
| 252 | NT | ++ | NT | NT |
| 253 | +++ | + | NT | NT |
| 254 | ++ | + | NT | NT |
| 255 | NT | + | NT | NT |
| 256 | +++ | + | NT | NT |
| 257 | ++++ | ++ | NT | NT |
| 258 | +++ | ++ | NT | NT |
| 259 | ++++ | ++ | NT | NT |
| 260 | +++ | + | NT | NT |
| 261 | + | NT | NT | NT |
| 262 | +++ | NT | NT | NT |
| 263 | +++ | + | NT | NT |
| 264 | +++ | + | NT | NT |
| 265 | +++ | NT | NT | NT |
| 266 | +++ | NT | NT | NT |
| 267 | ++ | NT | NT | NT |
| 268 | ++ | NT | NT | NT |
| 269 | ++ | NT | NT | NT |
| 270 | + | NT | NT | NT |
| 271 | + | NT | NT | NT |
| 272 | + | NT | NT | NT |
| 273 | ++++ | +++ | NT | NT |
| 274 | ++ | + | + | + |
| 275 | + | + | + | + |
| 276 | + | NT | + | + |
| 277 | ++ | + | NT | NT |
| 278 | ++ | + | NT | NT |
| 279 | +++ | ++ | NT | NT |
| 280 | ++ | + | NT | NT |
| 281 | ++ | + | NT | NT |
| 282 | ++ | + | NT | NT |
| 283 | ++++ | ++ | NT | NT |
| 284 | ++++ | +++ | NT | NT |
| 285 | ++++ | +++ | NT | NT |
| 286 | +++ | ++ | NT | NT |
| 287 | ++++ | ++ | NT | NT |
| 288 | ++++ | ++ | NT | NT |
| 289 | ++++ | +++ | NT | NT |
| 290 | ++++ | +++ | NT | NT |
| 291 | ++++ | ++ | NT | NT |
| 292 | NT | NT | NT | NT |
| 293 | ++++ | ++ | NT | NT |
| 294 | +++ | + | NT | NT |
| 295 | NT | +++ | NT | NT |
| 296 | ++++ | ++ | NT | NT |
| 297 | ++++ | ++ | NT | NT |
| 298 | +++ | + | NT | NT |
| 299 | +++ | ++ | NT | NT |
| 300 | ++++ | ++ | NT | NT |
| 301 | ++++ | +++ | NT | NT |
| 302 | NT | ++ | NT | NT |
| 303 | +++ | + | NT | NT |
| 304 | ++++ | +++ | NT | NT |
| 305 | +++ | ++ | NT | NT |
| 306 | +++ | + | NT | NT |
| 307 | ++++ | +++ | + | + |
| 308 | ++++ | ++++ | + | + |
| 309 | ++++ | ++++ | NT | NT |
| 310 | ++++ | +++ | NT | NT |
| 311 | ++++ | +++ | NT | NT |
| 312 | ++++ | +++ | NT | NT |
| 313 | +++ | + | NT | NT |
| 314 | +++ | + | NT | NT |
| 315 | +++ | + | NT | NT |
| 316 | ++++ | +++ | NT | NT |
| 317 | ++++ | ++ | NT | NT |
| 318 | +++ | + | NT | NT |
| 319 | +++ | + | NT | NT |
| 320 | ++++ | +++ | NT | NT |
| 321 | ++++ | +++ | NT | NT |
| 322 | ++++ | +++ | + | + |
| 323 | ++ | ++ | NT | NT |
| 324 | ++++ | +++ | NT | NT |
| 325 | ++++ | +++ | NT | NT |
| 326 | ++++ | +++ | NT | NT |
| 327 | ++++ | +++ | NT | NT |
| 328 | ++++ | +++ | NT | NT |
| 329 | ++++ | +++ | NT | NT |
| 330 | ++++ | +++ | NT | NT |
| 331 | ++ | + | NT | NT |
| 332 | ++ | + | NT | NT |
| 333 | ++ | + | NT | NT |
| 334 | +++ | + | NT | NT |
| 335 | +++ | + | NT | NT |
| 336 | ++ | + | NT | NT |
| 337 | +++ | ++ | NT | NT |
| 338 | ++ | ++ | NT | NT |
| 339 | +++ | + | NT | NT |
| 340 | +++ | + | NT | NT |
| 341 | + | + | NT | NT |
| 342 | ++ | + | NT | NT |
| 343 | ++ | + | NT | NT |
| 344 | ++ | + | NT | NT |
| 345 | ++ | + | NT | NT |
| 346 | ++ | + | NT | NT |
| 347 | ++ | + | NT | NT |
| 348 | ++ | + | NT | NT |
| 349 | ++ | + | NT | NT |
| 350 | ++ | + | NT | NT |
| 351 | ++ | + | NT | NT |
| 352 | ++ | + | NT | NT |
| 353 | ++ | + | NT | NT |
| 354 | ++ | + | NT | NT |
| 355 | ++++ | ++ | NT | NT |
| 356 | +++ | + | NT | NT |
| 357 | +++ | + | NT | NT |
| 358 | + | + | NT | NT |
| 359 | +++ | + | NT | NT |
| 360 | NT | + | NT | NT |
| 361 | +++ | NT | NT | NT |
| 362 | NT | ++ | NT | NT |
| 363 | ++ | + | NT | NT |
| 364 | ++ | + | NT | NT |
| 365 | NT | ++ | NT | NT |
| 366 | NT | ++ | NT | NT |
| 367 | ++ | + | NT | NT |
| 368 | ++ | + | NT | NT |
| 369 | ++ | + | NT | NT |
| 370 | ++ | + | NT | NT |
| 371 | + | + | NT | NT |
| 372 | ++ | + | NT | NT |
| 373 | ++ | + | NT | NT |
| 374 | +++ | + | NT | NT |
| 375 | ++ | + | NT | NT |
| 376 | ++ | + | NT | NT |
| 377 | +++ | + | NT | NT |
| 378 | ++ | + | NT | NT |
| 379 | +++ | + | NT | NT |
| 380 | +++ | NT | NT | NT |
| 381 | ++++ | ++++ | NT | NT |
| 382 | ++++ | ++++ | NT | NT |
| 383 | ++++ | +++ | NT | NT |
| 384 | ++++ | +++ | + | + |
| 385 | ++++ | +++ | NT | NT |
| 386 | ++++ | ++++ | + | + |
| 387 | ++++ | ++++ | NT | NT |
| 388 | ++++ | ++++ | NT | NT |
| 389 | ++++ | ++++ | NT | NT |
| 390 | ++++ | ++++ | NT | NT |
| 391 | ++++ | +++ | NT | NT |
| 392 | ++++ | ++++ | NT | NT |
| 393 | ++++ | NT | NT | NT |
| 394 | ++++ | ++++ | NT | NT |
| 395 | ++++ | +++ | NT | NT |
| 396 | +++ | + | NT | NT |
| 397 | ++++ | ++ | NT | NT |
| 398 | ++++ | +++ | NT | NT |
| 399 | ++++ | +++ | NT | NT |
| 400 | ++++ | +++ | NT | NT |
| 401 | +++ | + | NT | NT |
| 402 | +++ | + | NT | NT |
| 403 | ++++ | ++ | NT | NT |
| 404 | +++ | + | NT | NT |
| 405 | ++ | + | NT | NT |
| 406 | ++ | + | NT | NT |
| 407 | ++++ | ++ | NT | NT |
| 408 | +++ | ++ | NT | NT |
| 409 | +++ | + | NT | NT |
| 410 | + | ++ | NT | NT |
| 411 | ++ | + | NT | NT |

What is claimed is:

1. A compound of Formula (I): 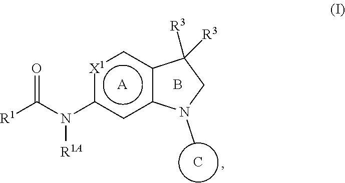
or a pharmaceutically acceptable salt thereof, wherein:
ring A is an aromatic or heteroaromatic ring fused with ring B that is a 5-membered heterocycle;
X¹ is N or CH;
ring C represents is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{C};
each R^{C} is independently halo, —CN, —NR^{N1}R^{N2}, —NR^{N3}—C(O)—R⁷, —C(O)—NR^{N3}R^{N4}, —NR^{N4}—SO₂—R⁷, —C(O)—R⁷, —C(O)—OH, —C(O)—OR⁷, —SR⁷, —SO₂—R⁷, —OR^{O1}, C₁₋₆ alkyl, C₂₋₆alkenyl, phenyl, 5 to 6 membered heteroaryl, 3 to 7 membered monocyclic carbocyclyl or 4 to 9 membered monocyclic or bicyclic heterocyclyl, wherein the C₁₋₆ alkyl, C₁₋₆alkenyl, phenyl, 5 to 6 membered heteroaryl, 3 to 7 membered monocyclic carbocyclyl and 4 to 9 membered monocyclic or bicyclic heterocyclyl represented by R^{C} are each optionally substituted with one or more R^{C1};
each R^{C1} is independently halo, oxo, —CN, —OR^{O1}, —NR^{N3}R^{N4}, —C(O)—R⁷, —C(O)—OR^{O3}, —SO₂—R⁷, C₁₋₆ alkyl, phenyl, 3 to 7 membered monocyclic carbocyclyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —ORO, —NR^{N3}R^{N4}, C₁₋₆ alkyl, C₁₋₄ haloalkyl, phenyl, 5 to 6 membered heteroaryl, 3 to 7 membered monocyclic carbocyclyl, and 4 to 8 membered monocyclic heterocyclyl;
R¹ is H, C₁₋₆ alkyl, —OR², —NR²R⁴, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
each R² and R⁴ are independently H, C₁₋₄alkyl, or 3 to 4 membered monocyclic carbocyclyl;
each R³ is independently H, halo, or C₁₋₃ alkyl;
alternatively two R³, together with the atom to which they are attached, form a 3 to 7 membered monocyclic carbocyclyl or 4 to 9 membered monocyclic heterocyclyl, each of which is optionally substituted by one or more R⁵;
each R⁵ is independently H, halo, —CN, —OR^{O1}, —NR^{N3}R^{N4}, C₁₋₃ alkyl, —C(O)—R⁷, —C(O)—OR^{O3}, —C(O)—NR^{N1}R^{N2}, —NR^{N3}—C(O)—R⁷, SO₂—R⁷, phenyl, 5 to 6 membered heteroaryl, or 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁵ are each optionally substituted by one or more substituents independently selected from halo, oxo, —CN, —OR^{O1}, —NR^{N1}R^{N2}, C₁₋₆ alkyl, C₁₋₄ haloalkyl, 3 to 7 membered monocyclic carbocyclyl and 4 to 7 membered monocyclic heterocyclyl;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{N3}R^{N4}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R^{O1} is independently H, C₁₋₆ alkyl, phenyl, 5 to 6 membered heteroaryl, 3 to 7 membered monocyclic carbocyclyl, or 4 to 8 membered monocyclic or bicyclic heterocyclyl, wherein the C₁₋₆ alkyl, phenyl, 5 to 6 membered heteroaryl, 3 to 8 membered monocyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄alkyl, C₁₋₄alkyl-C₁₋₄alkoxy, C₁₋₄ haloalkyl, phenyl, 5 to 6 membered heteroaryl, 3 to 7 membered monocyclic carbocylyl or 4 to 7 membered monocyclic or bicyclic heterocyclyl, wherein the 4 to 7 membered monocyclic or bicyclic heterocyclyl is optionally substituted with C₁₋₄alkyl or C₁₋₄alkoxy;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H or C₁₋₆ alkyl, wherein the C₁-s alkyl represented by R^{N1} and R^{N2} are each optionally substituted with halo, C₁₋₄alkoxy or —NR^{N3}R^{N4}, or
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and C₁₋₄alkoxy;
each R^{N3} is independently H or C₁₋₆ alkyl; and
each R^{N4} is independently H or C₁₋₆ alkyl.

2. The compound of claim 1, or a pharmaceutically acceptable salt thereof, wherein:
ring A is an aromatic or heteroaromatic ring fused with ring B that is a 5-membered heterocycle;
X¹ is N or CH;
ring C represents is phenyl, 5 to 10 membered monocyclic or bicyclic heteroaryl, 3 to 7 membered monocyclic carbocyclyl or 4 to 7 membered monocyclic heterocyclyl, each of which is optionally substituted by one or more R^{C};
each R^{C} is independently halo, —CN, —NR^{N1}R^{N2}, —NR^{N3}—C(O)—R⁷, —C(O)—NR^{N3}R^{N4}, —NR^{N4}—SO₂—R⁷, —C(O)—R⁷, —SR⁷, —SO₂—R⁷, —OR^{O1}, C₁₋₆ alkyl, phenyl, 5 to 6 membered heteroaryl, 3 to 7 membered monocyclic carbocyclyl or 4 to 9 membered monocyclic or bicyclic heterocyclyl, wherein the C₁₋₆ alkyl, phenyl, 5 to 6 membered heteroaryl, 3 to 7 membered monocyclic carbocyclyl and 4 to 9 membered monocyclic or bicyclic heterocyclyl represented by R^{C} are each optionally substituted with one or more R^{C1};
each R^{C1} is independently halo, oxo, —CN, —ORO, —NR^{N3}R^{N4}, —C(O)—R⁷, —C(O)—OR^{O3}, —SO₂—R⁷, C₁₋₆ alkyl, phenyl, 3 to 7 membered monocyclic carbocyclyl, 5 to 6 membered heteroaryl, or 4 to 7 membered monocyclic heterocyclyl, wherein the C₁₋₆ alkyl, phenyl, 5 to 6 membered heteroaryl and 4 to 7 membered monocyclic heterocyclyl represented by R^{C1} are each optionally substituted by one or more substituents independently selected from halo, oxo, —CN, —OR^{O1}, —NR^{N3}R^{N4}, C₁₋₆ alkyl, C₁₋₄ haloalkyl, phenyl, 5 to 6 membered heteroaryl, 3 to 7 membered monocyclic carbocyclyl, and 4 to 8 membered monocyclic heterocyclyl;
R¹ is H, C₁₋₆ alkyl, —OR², —NR²R⁴, 3 to 7 membered monocyclic carbocyclyl, or 4 to 7 membered monocyclic heterocyclyl, wherein the C₁₋₆ alkyl, 3 to 7 membered monocyclic carbocyclyl and 4 to 7 membered monocyclic heterocyclyl represented by R¹ are each optionally substituted by one or more R⁸;
R^{1A} is H or C₁₋₃alkyl;
each R² and R⁴ are independently H, C₁₋₄alkyl, or 3 to 4 membered monocyclic carbocyclyl;
each R³ is independently H, halo, or C₁₋₃ alkyl;
alternatively two R³, together with the atom to which they are attached, form a 3 to 7 membered monocyclic carbocyclyl or 4 to 9 membered monocyclic heterocyclyl, each of which is optionally substituted by one or more R⁵;
each R⁵ is independently H, halo, —CN, —OR^{O1}, —NR^{N3}R^{N4}, C₁₋₃ alkyl, —C(O)—R⁷, —C(O)—OR^{O3}, —C(O)—NR^{N1}R^{N2}, —NR^{N3}—C(O)—R⁷, SO₂—R⁷, phenyl, 5 to 6 membered heteroaryl, or 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R⁵ are each optionally substituted by one or more substituents independently selected from halo, oxo, —CN, —OR^{O1}, —NR^{N1}R^{N2}, C₁₋₆ alkyl, C₁₋₄ haloalkyl, 3 to 7 membered monocyclic carbocyclyl and 4 to 7 membered monocyclic heterocyclyl;
each R⁷ is independently C₁₋₆ alkyl, phenyl, 5 to 6 membered heteroaryl, 3 to 7 membered monocyclic carbocyclyl or 4 to 7 membered monocyclic heterocyclyl; wherein the C₁₋₆ alkyl, phenyl, 5 to 6 membered heteroaryl, 3 to 7 membered monocyclic carbocyclyl and 4 to 7 membered monocyclic heterocyclyl represented by R⁷ are each optionally substituted by one or more substituents independently selected from halo, oxo, —CN, —OR^{O1}, —NR^{N3}R^{N4}, C₁₋₆ alkyl, C₁₋₄ haloalkyl, 3 to 7 membered monocyclic carbocyclyl and 4 to 7 membered monocyclic heterocyclyl;
each R⁸ is independently halo, oxo, —CN, —OR^{O1}, —NR^{N1}R^{N2}, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl;
each R^{O1} is independently H, C₁₋₆ alkyl, phenyl, 5 to 6 membered heteroaryl, 3 to 7 membered monocyclic carbocyclyl, or 4 to 7 membered monocyclic or bicyclic heterocyclyl, wherein the C₁₋₆ alkyl, phenyl, 5 to 6 membered heteroaryl, 3 to 7 membered monocyclic carbocyclyl, and 4 to 7 membered monocyclic or bicyclic heterocyclyl represented by R^{O1} are each optionally substituted by one or more R^{O2};
each R^{O2} is independently halo, OH, —CN, C₁₋₄ alkoxy, C₁₋₄ haloalkyl, phenyl, 5 to 6 membered heteroaryl, 3 to 7 membered monocyclic carbocylyl or 4 to 7 membered monocyclic heterocyclyl;
each R^{O3} is independently H, C₁₋₆ alkyl, C₁₋₄ haloalkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O3} are each optionally substituted by one or more R^{O2};
R^{O4} is H, C₁₋₆ alkyl, phenyl, 5 to 6 membered heteroaryl, 3 to 7 membered monocyclic carbocyclyl or 4 to 7 membered monocyclic heterocyclyl, wherein the C₁₋₆ alkyl, phenyl, 5 to 6 membered heteroaryl, 3 to 7 membered monocyclic carbocyclyl and 4 to 7 membered monocyclic heterocyclyl represented by R^{O4} are each optionally substituted by one or more substituents independently selected from halo, oxo, —CN, —OR^{O1}, —N^{N1}R^{N2}, C₁₋₆ alkyl, C₁₋₄ haloalkyl, phenyl, 5 to 6 membered heteroaryl, 3 to 7 membered monocyclic carbocyclyl and 4 to 7 membered monocyclic heterocyclyl;
R^{N1} and R^{N2} are each independently H or C₁₋₆ alkyl, wherein the C₁₋₆ alkyl represented by R^{N1} and R^{N2} are each optionally substituted with halo, C₁₋₄alkoxy or —NR^{N3}R^{N4}, or
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and C₁₋₄alkoxy;
each R^{N3} is independently H or C₁₋₆ alkyl; and
each R^{N4} is independently H or C₁₋₆ alkyl.

3. The compound of claim 1 or 2, or a pharmaceutically acceptable salt thereof, wherein R^{1A} is H or —CH₃.

4. The compound of any one of claims 1 to 3, or a pharmaceutically acceptable salt thereof, wherein each R³ is independently H or —CH₃.

5. The compound of any one of claims 1 to 3, or a pharmaceutically acceptable salt thereof, wherein two R³, together with the atom to which they are attached, form C₃₋₆ cycloalkyl or 5 to 6 membered monocyclic heterocyclyl, each of which is optionally substituted by one to three R⁵.

6. The compound of claim 5, or a pharmaceutically acceptable salt thereof, wherein two R³, together with the atom to which they are attached, form cyclopropane, cyclobutane, cyclopentane, tetrahydropyran, or piperidine, each of which is optionally substituted by one to three R⁵.

7. The compound of claim 5, or a pharmaceutically acceptable salt thereof, wherein two R³ together with the atom to which they are attached form: 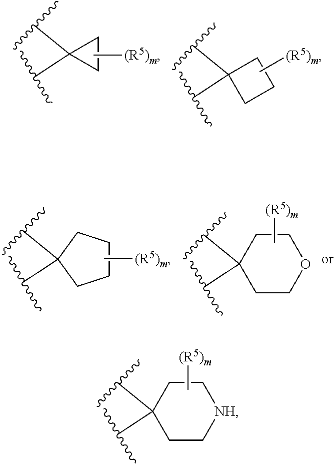
wherein m is 0, 1, 2 or 3, and [symbol] represents a bond within ring B.

8. The compound of claim 1 or 2, wherein the compound is represented by formula (III), (IV), (V), (VI), (VII), (VIII) or (IX): 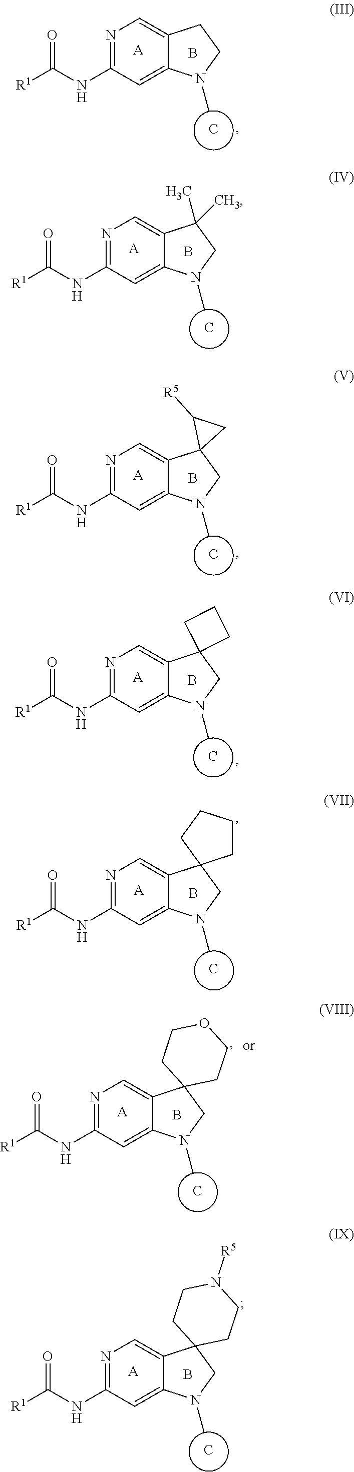
or a pharmaceutically acceptable salt thereof.

9. The compound of any one of claims 1, 2, 3 and 5-8, or a pharmaceutically acceptable salt thereof, wherein each R⁵ is independently H, —CN, or CH₃.

10. The compound of any one of claims 1-9, or a pharmaceutically acceptable salt thereof, wherein ring C is 5 to 9 membered monocyclic or bicyclic heteroaryl optionally substituted by one to three R^{C}.

11. The compound of claim 10, or a pharmaceutically acceptable salt thereof, wherein ring C is pyrazine, pyrozolo[1,5-a]pyrimidine, pyridine, pyrimidine, pyrimidinone or thiazole, each of which is optionally substituted by one or three R^{C}.

12. The compound of claim 10, or a pharmaceutically acceptable salt thereof, wherein ring C is selected from: 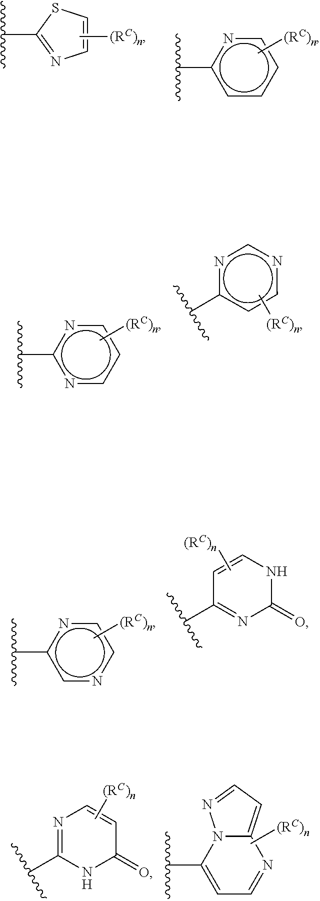
and wherein [symbol] represents a bond to ring B, and n is 0, 1, 2, or 3.

13. The compound of claim 10, or a pharmaceutically acceptable salt thereof, wherein ring C is selected from: 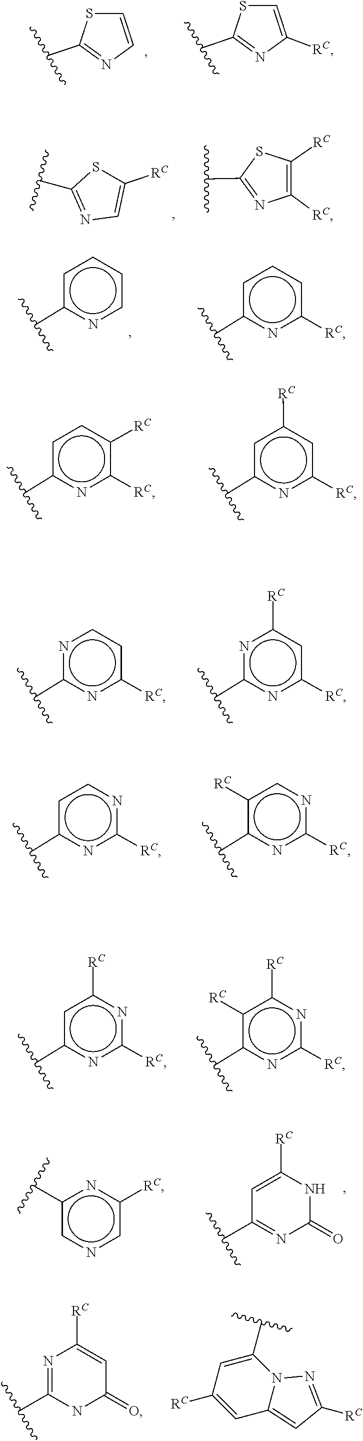
and wherein [symbol] represents a bond to ring B, and the one to three R^{C} groups in ring C may be the same or different.

14. The compound of any one of claims 1-13, or a pharmaceutically acceptable salt thereof, wherein:
each R^{C} is independently halo, —NR^{N1}R^{N2}, —OR^{O1}, —SR⁷, —SO₂—R⁷, —C(O)—NR^{N3}R^{N4}, —C(O)—R⁷, —C(O)H, —C(O)OH, —C(O)OR⁷, C₁₋₆ alkyl, C₂₋₄alkenyl, C₃₋₆ cycloalkyl, 4 to 9 membered monocyclic or bicyclic heterocyclyl, phenyl or 5 to 6 membered heteroaryl, wherein the C₁₋₆ alkyl, C₂₋₄alkenyl, C₃₋₆ cycloalkyl, 4 to 9 membered monocyclic or bicyclic heterocyclyl, phenyl and 5 to 6 membered heteroaryl represented by R^{C} are each optionally substituted with one to three R^{C1};
R⁷ is C₁₋₃alkyl;
R^{O1} is H, C₁₋₄alkyl, C₁₋₄alkyl, C₃₋₆cycloalkyl, 4 to 8 membered monocyclic or bicyclic heterocyclyl, 5 to 6 membered heteroaryl, wherein C₁₋₄alkyl represented by R^{O1} is optionally substituted with one to three substituents independently selected from halo, OH, CN, C₁₋₃alkoxy, C₃₋₆cycloalkyl, and 4 to 6 membered monocyclic heterocyclyl optionally substituted with C₁₋₃alkyl or C₁₋₃alkoxy, and wherein C₃₋₆cycloalkyl, 4 to 8 membered monocyclic or bicyclic heterocyclyl, and 5 to 6 membered heteroaryl represented by R^{O1} are each optionally substituted with one to two substitutents independently selected from halo, C₁₋₃alkyl, C₁₋₃alkyl-C₁₋₃alkoxy, CN, OH and C₁₋₃alkoxy;
R^{N1} and R^{N2} are each independently H or C₁₋₆ alkyl, wherein the C₁₋₆ alkyl represented by R^{N1} and R^{N2} are each optionally substituted with halo, C₁₋₄alkoxy or —NR^{N3}R^{N4}, or
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl, and —C₁₋₄alkoxy; R^{N1} and R^{N2} are each independently H or C₁₋₄ alkyl, wherein the C₁₋₄ alkyl represented by R^{N1} and R^{N2} are each optionally substituted with halo, C₁₋₃alkoxy or —NR^{N3}R^{N4}, or
each R^{N3} is independently H or C₁₋₃alkyl; and
each R^{N4} is independently H or C₁₋₃alkyl.

15. The compound of any one of claims 1-13, or a pharmaceutically acceptable salt thereof, wherein:
each R^{C} is independently halo, —NR^{N1}R^{N2}, —OR^{O1}, —SR⁷, —SO₂—R⁷, —C(O)—NR^{N3}R^{N4}, —C(O)—R⁷, C₁₋₃ alkyl, C₃₋₆ cycloalkyl, 4 to 9 membered monocyclic or bicyclic heterocyclyl, phenyl or 5 to 6 membered heteroaryl, wherein the C₁₋₃ alkyl, C₃₋₆ cycloalkyl, 4 to 9 membered monocyclic or bicyclic heterocyclyl, phenyl and 5 to 6 membered heteroaryl represented by R^{C} are each optionally substituted with one to three R^{C1};
R⁷ is C₁₋₃alkyl;
R^{O1} is H, C₁₋₄alkyl, C₁₋₄alkyl, C₃₋₆cycloalkyl, 4 to 6 membered monocyclic heterocyclyl, 5 to 6 membered heteroaryl, wherein C₁₋₄alkyl represented by R^{O1} is optionally substituted with one to three substituents independently selected from halo, CN, C₁₋₃alkoxy, C₃₋₆cycloalkyl, 4 to 6 membered monocyclic heterocyclyl, and wherein C₃₋₆cycloalkyl, 4 to 6 membered monocyclic heterocyclyl, and 5 to 6 membered heteroaryl represented by R^{O1} are each optionally substituted with one to two substitutents independently selected from halo, C₁₋₃alkyl, CN, OH and C₁₋₃alkoxy;
R^{N1} is H or C₁₋₃alkyl and R^{N2} is ring D or —C₁₋₃alkylene-ring D;
Ring D is 3 to 7 membered monocyclic or bicyclic carbocyclyl, or 4 to 7 membered monocyclic heterocyclyl, wherein the 3 to 7 membered monocyclic carbocyclyl and 4 to 7 membered monocyclic heterocyclyl represented by ring D are each optionally substituted with one or two substituents independently selected from halo, C₁₋₆ alkyl and —C₁₋₄alkoxy;
R^{N1} and R^{N2} are each independently H or C₁₋₄ alkyl, wherein the C₁₋₄ alkyl represented by R^{N1} and R^{N2};
each R^{N3} is independently H or C₁₋₃alkyl; and
each R^{N4} is independently H or C₁₋₃alkyl.

16. The compound of claim 14, or a pharmaceutically acceptable salt thereof, wherein ring D is cyclobutane, cyclopropane, bicyclo[1.1.1]pentane, spiro[2.2]pentane, azetidine, oxetane, tetrahydrofuran, tetrahydropyran, tetrahydrothiophene or thietane, each of which is optionally substituted with one or two substitutents independently selected from halo, C₁₋₂ alkyl and C₁₋₂alkoxy.

17. The compound of claim 16, or a pharmaceutically acceptable salt thereof, wherein ring D is one of the following: 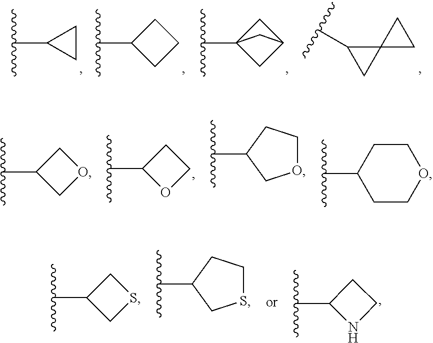
each of which is optionally substituted with one or two substituents independently selected from F, —CH₃ and —OCH₃.

18. The compound of claim 14, or a pharmaceutically acceptable salt thereof, wherein the 4 to 8 membered monocyclic or bicyclic heterocyclyl represented by R^{O1} or a substituent of C₁₋₄alkyl represented by R^{O1} is oxetane, azetindine, pyrrolidine, 2-oxaspiro[3.3]heptanyl, or tetrahydrofuran, each of which is optionally substituted with one to two substituents independently selected with C₁₋₃alkyl, halo, CN, OH, C₁₋₃alkyl-C₁₋₃alkoxy, or C₁₋₃alkoxy; and the 5 to 6 membered heteroaryl represented by R^{O1} is pyrazole, triazole, pyridine, pyrazine, pyrimidine, or pyridazine, each of which is optionally substituted with one to two substituents independently selected with C₁₋₃alkyl, halo, CN, OH or C₁₋₃alkoxy.

19. The compound of claim 15, or a pharmaceutically acceptable salt thereof, wherein the 4 to 6 membered monocyclic heterocyclyl represented by R^{O1} or a substituent of C₁₋₄alkyl represented by R^{O1} is oxetane, azetindine or tetrahydrofuran, each of which is optionally substituted with one to two substituents independently selected with C₁₋₃alkyl, halo, CN, OH or C₁₋₃alkoxy; and the 5 to 6 membered heteroaryl represented by R^{O1} is pyrazole, triazole, pyridine, pyrazine, pyrimidine, or pyridazine, each of which is optionally substituted with one to two substituents independently selected with C₁₋₃alkyl, halo, CN, OH or C₁₋₃alkoxy.

20. The compound of claim 19, or a pharmaceutically acceptable salt thereof, wherein the 4 to 6 membered monocyclic heterocyclyl is: 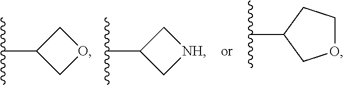
each of which is optionally substituted with one or two substituents independently selected from F, —CN, —CH₃, —CH₂CH₃, OH, —CH₂OCH₃, and —OCH₃; and
the 5 to 6 membered heteroaryl is: 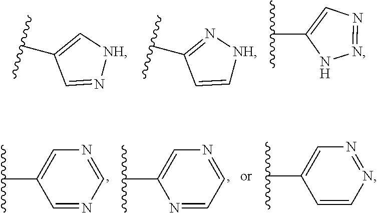
each of which is optionally substituted with one or two substituents independently selected from F, —CN, —CH₃, —CH₂CH₃, OH and —OCH₃.

21. The compound of claim 15, or a pharmaceutically acceptable salt thereof, wherein:
each R^{C} is independently selected from F, —Cl, —CH₃, —CHF₂, —CF₃, —CH₂CH₃, —CH₂CH₂CH₃, —CH₂CH₂CH₂CH₃, —CH₂CH₂CH₂CH₂CH₃, —CF₂CH₃, —CF₂CFH₂, —CFHCFH₂, —CF₂CH₂CH₃, —CF₂CH₂OH, —CF₂CH₂OCH₃, —CH(CH₃)₂, —CF(CH₃)₂, —C(CH₃)₃, —CF₂C(O)OCH₂CH₃, —CH₂OH, —CH(OH)CH₃, —CH₂OCH₃, —CH₂CH₂CH₂OH, —CH₂CH₂OCH₂CH₃, —CH₂CH₂CH₂OCH₃, —CH₂CH₂C(CH₃)(OH)CH₂CH₃, —CH₂CH₂CH₂OC(CH₃)₃, —CH₂CH₂C(CH₃)₂OH, —CH(CH₃)OCH₃, —C(CH₃)₂OCH₃, —CH₂OCH₂CH₃, —CH₂-cyclopropyl, —CH(CH₃)—SO₂CH₃, —CH₂CH₂—SO₂CH₃, —CH₂NHCH₃, —CH₂CN, —CH(CH₃)CN, —CH═CH—OCH₂CH₃, OH, —OCH₃, —OCH₂CH₃, —OCH(CH₃)₂, —OCH₂CF₃, —OCH₂CF₂CH₃, —OCH₂CH₂OCH₃, —OCH₂CH₂F, —OCH₂CH₂CH₂OCH₃, —OCH₂CH₂N(CH₃)₂, —OCH(CH₃)CH₂OCH₃, —OCH(CH₃)CN, —OCH₂C(CH₃)₂OH, —OCH₂C(CH₃)₂OCH₃, —OCH(cyclopropyl)(CN), —C(O)NH₂, —C(O)NHCH₃, —C(O)N(CH₃)₂, —C(O)H, —C(O)CH₃, —C(O)OH, —C(O)OCH₂CH₃, —NH₂, —NHCH₃, —NHCH₂CH₃, —NHCH₂CH₂CH₃, —NHCH(CH₃)₂, —N(CH₃)₂, —N(CH₃)CH(CH₃)₂, —NHCH₂CH₂CH₂F, —NHCH₂CHF₂, —NHCH₂C(F)CH₃, —NHCH₂CH₂OCH₃, —NHCH(CH₃)CH₂OCH₃, —NHCH₂CH(CH₃)N(CH₃)₂, —NHCH₂C(CH₃)₂OCH₃, —NHCH₂CH₂C(F)CH₃, —NHCH₂CH(CH₃)OCH₃, —NHCH(CH₃)CH(CH₃)OCH₃, —N(CH₃)CH₂CH₂OCH₃, —NHC(O)CH₃, —SCH₃, —SO₂CH₃,

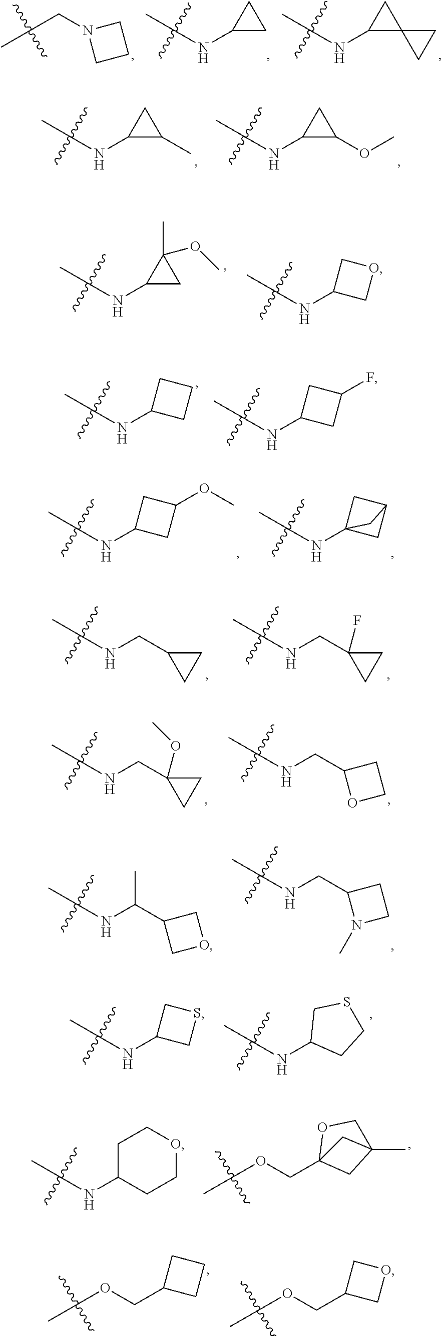

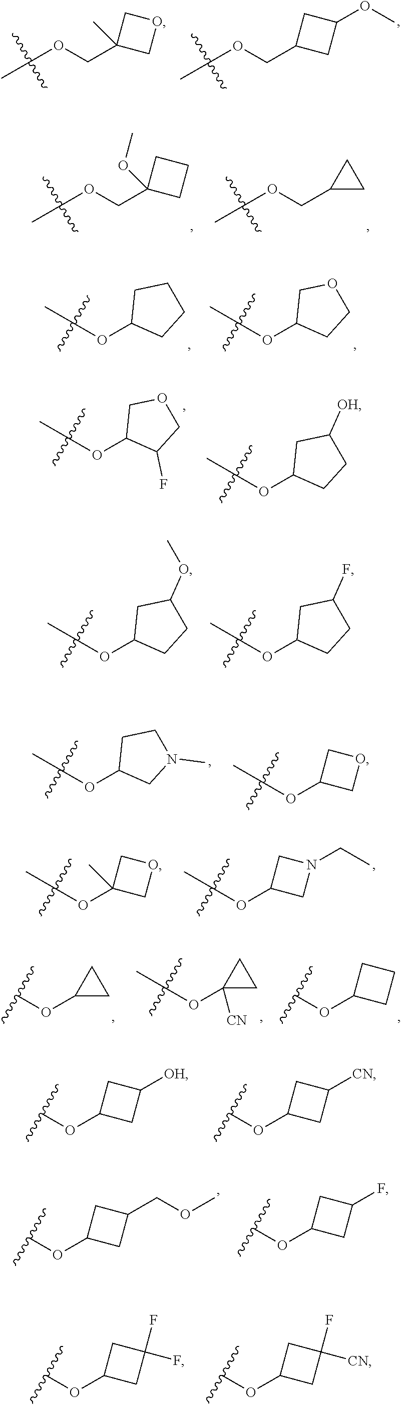

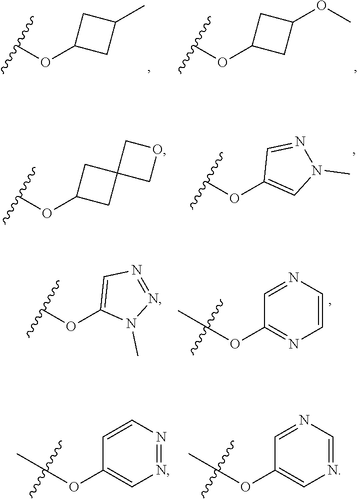

22. The compound of claim 15, or a pharmaceutically acceptable salt thereof, wherein: each R^{C} is independently selected from F, —CH₃, —CHF₂, —CF₃, —CH₂CH₃, —CH₂CH₂CH₃, —CH₂CH₂CH₂CH₃, —CF₂CH₃, —CF₂CFH₂, —CFHCFH₂, —CH(CH₃)₂, —CF(CH₃)₂, —C(CH₃)₃, —CH₂OH, —CH₂OCH₃, —CH₂CH₂CH₂OCH₃, —CH(CH₃)OCH₃, —C(CH₃)₂OCH₃, —CH₂OCH₂CH₃, —CH₂-cyclopropyl, —CH(CH₃)—SO₂CH₃, OH, —OCH₃, —OCH₂CH₂OCH₃, —OCH₂CH₂CH₂OCH₃, —OCH₂CH₂N(CH₃)₂, —OCH(CH₃)CH₂OCH₃, —OCH(CH₃)CN, —C(O)NH₂, —C(O)NHCH₃, —C(O)N(CH₃)₂, —C(O)H, —C(O)CH₃, —NH₂, —NHCH₃, —NHCH₂CH₃, —NHCH₂CH₂CH₃, —NHCH(CH₃)₂, —N(CH₃)₂, —N(CH₃)CH(CH₃)₂, —NHCH₂CH₂CH₂F, —NHCH₂CHF₂, —NHCH₂C(F)CH₃, —NHCH₂CH₂OCH₃, —NHCH₂CH(CH₃)N(CH₃)₂, —NHCH₂C(CH₃)₂OCH₃, —NHCH₂CH₂C(F)CH₃, —NHCH₂CH(CH₃)OCH₃, —NHCH(CH₃)CH(CH₃)OCH₃, —N(CH₃)CH₂CH₂OCH₃, —NHC(O)CH₃, —SCH₃, —SO₂CH₃,

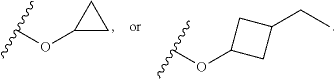

23. The compound of claim 15, or a pharmaceutically acceptable salt thereof, wherein:
each C₃₋₆cycloalkyl represented by R^{C} is selected from cyclobutane, cyclopentane and cyclopropane;
each 4 to 9 membered monocyclic or bicyclic heterocyclyl represented by R^{C} is independently selected from azetidine, morpholine, octahydropyrrolo[3,4-b]pyrrole, octahydropyrrolo[3,4-c]pyrrole, oxaazaspiro[3.5]nonane, oxabicyclo[2.1.1]hexane, oxabicyclo[2.2.1]heptane, oxetane, piperidine, piperazine, pyrrolidine, diydrofuran, tetrahydrofuran, tetrahydrothiophene dioxide and tetrahydropyran; and
each of the 5 to 6 membered heteroaryl represented by R^{C} is independently selected from oxazole, pyrazole, pyridine, pyrimidine, triazole, and thiazole;
and wherein each of the C₃₋₆ cycloalkyl, 4 to 9 membered monocyclic or bicyclic heterocyclyl and 5 to 6 membered heteroaryl is optionally substituted with one to three R^{C1}.

24. The compound of claim 15, or a pharmaceutically acceptable salt thereof, wherein:
each C₃₋₆cycloalkyl represented by R^{C} is selected from cyclobutane, cyclopentane and cyclopropane;
each 4 to 9 membered monocyclic or bicyclic heterocyclyl represented by R^{C} is independently selected from azetidine, morpholine, octahydropyrrolo[3,4-b]pyrrole, octahydropyrrolo[3,4-c]pyrrole, oxaazaspiro[3.5]nonane, oxabicyclo[2.2.1]heptane, oxetane, piperidine, piperazine, pyrrolidine, tetrahydrofuran, tetrahydrothiophene dioxide and tetrahydropyran; and
each of the 5 to 6 membered heteroaryl represented by R^{C} is independently selected from oxazole, pyrazole, pyridine, pyrimidine and thiazole;
and wherein each of the C₃₋₆ cycloalkyl, 4 to 9 membered monocyclic or bicyclic heterocyclyl and 5 to 6 membered heteroaryl is optionally substituted with one to three R^{C1}.

25. The compound of claim 15, or a pharmaceutically acceptable salt thereof, wherein:
each of the C₃₋₆cycloalkyl represented by R^{C} is independently selected from 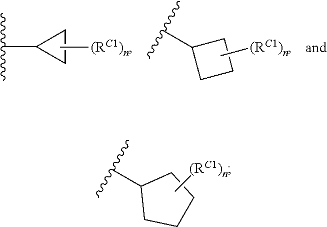
each of the 4 to 9 membered monocyclic heterocyclyl represented by R^{C} is independently selected from 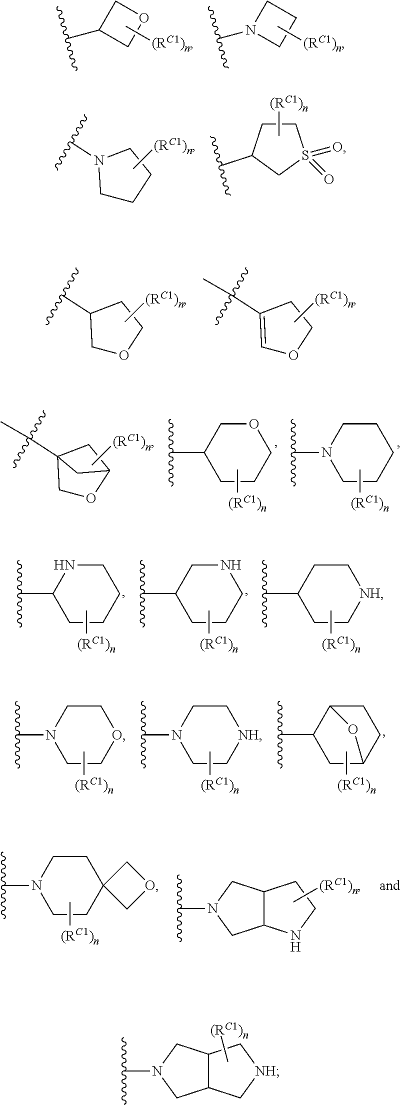
each of the phenyl represented by R^{C} is: 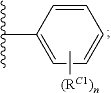
and
each of the 5 to 6 membered heteroaryl represented by R^{C} is independently selected from 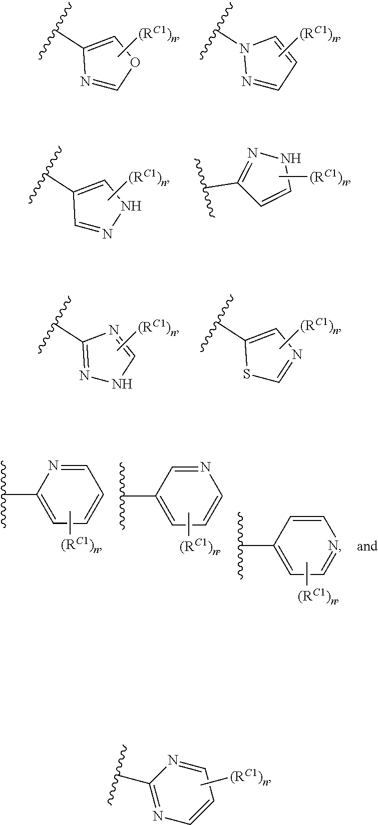
and wherein [symbol] represents a bond to ring C, and n is 0, 1, 2 or 3 as valency permits.

26. The compound of claim 15, or a pharmaceutically acceptable salt thereof, wherein:
each of the C₃₋₆cycloalkyl represented by R^{C} is independently selected from 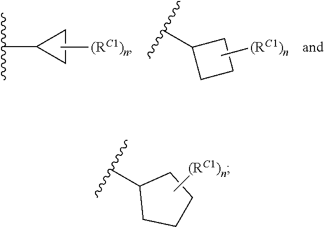
each of the 4 to 9 membered monocyclic heterocyclyl represented by R^{C} is independently selected from 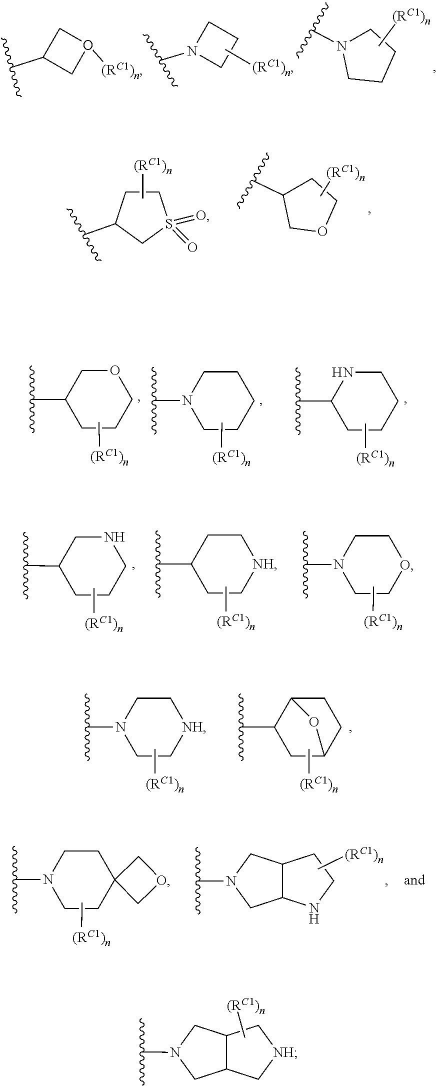
each of the phenyl represented by R^{C} is: 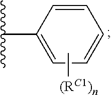
and
each of the 5 to 6 membered heteroaryl represented by R^{C} is independently selected from 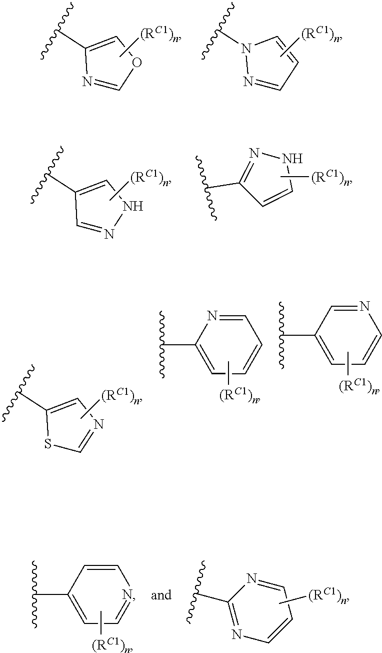
and wherein [symbol] represents a bond to ring C, and n is 0, 1, 2 or 3 as valency permits.

27. The compound of claim 15, or a pharmaceutically acceptable salt thereof, wherein:
each of the C₃₋₆ cycloalkyl represented by R^{C} is independently selected from: 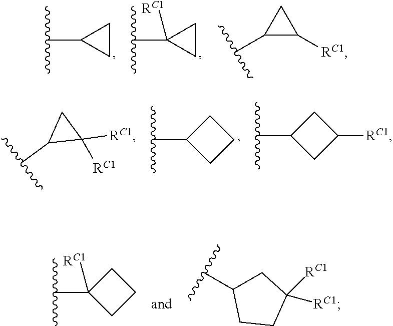
each of the 4 to 6 membered monocyclic heterocyclyl represented by R^{C} is independently selected from: 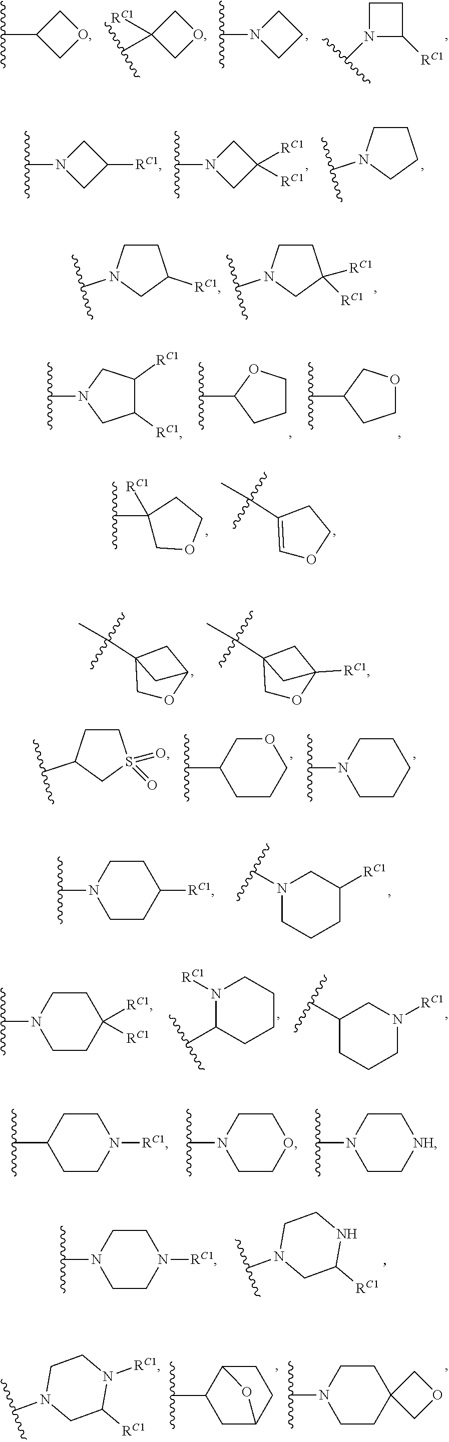 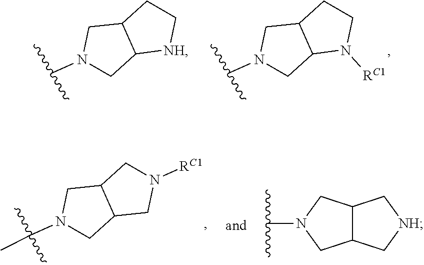
and
each of the phenyl represented by R^{C} is independently selected from 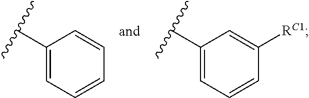
each of the 5 to 6 membered heteroaryl represented by R^{C} is independently selected from 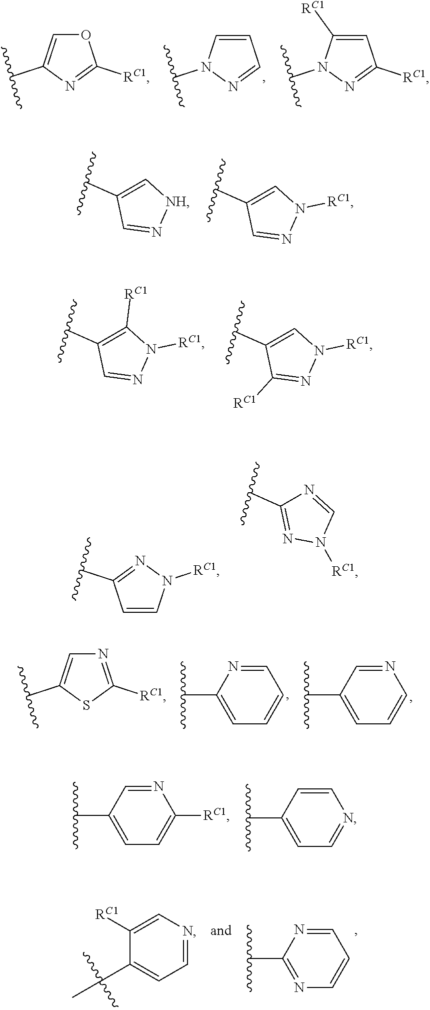
and wherein [symbol] represents a bond ring C.

28. The compound of claim 15, or a pharmaceutically acceptable salt thereof, wherein:
each of the C₃₋₆ cycloalkyl represented by R^{C} is independently selected from: 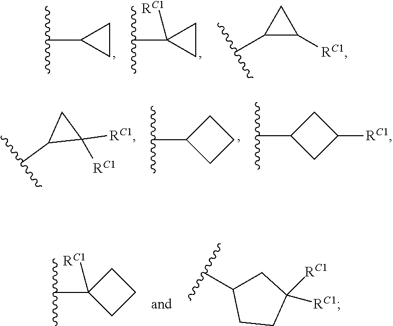
each of the 4 to 6 membered monocyclic heterocyclyl represented by R^{C} is independently selected from: 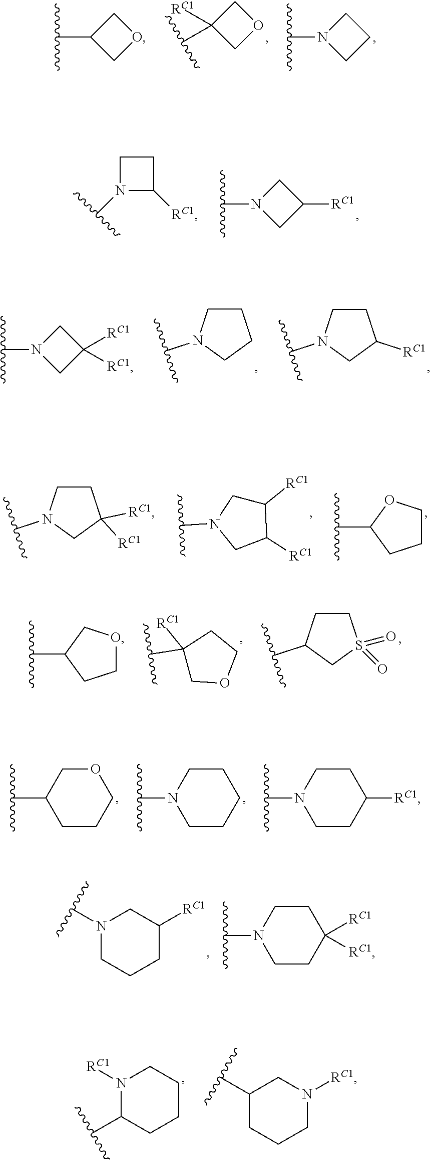 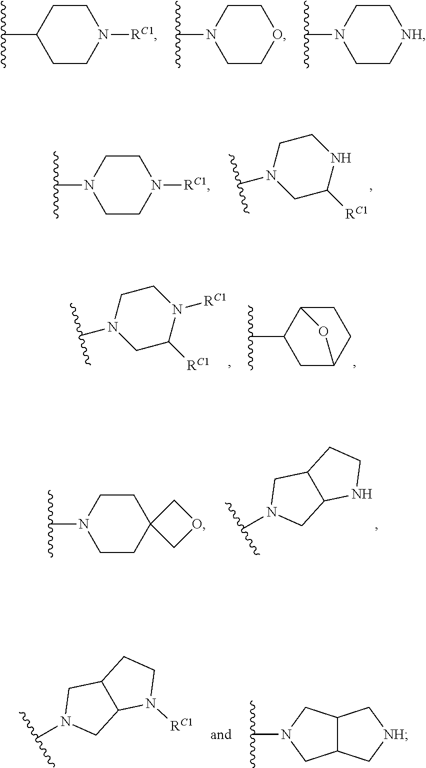
and
each of the phenyl represented by R^{C} is independently selected from 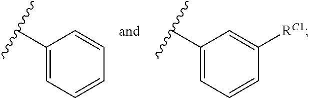
each of the 5 to 6 membered heteroaryl represented by R^{C} is independently selected from 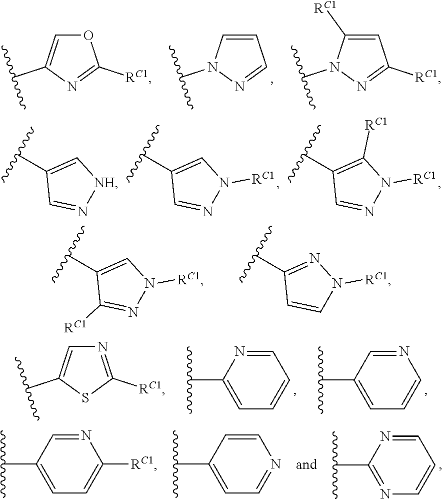
and wherein [symbol] represents a bond ring C.

29. The compound of any one of claims 1-28, or a pharmaceutically acceptable salt thereof, wherein each R^{C1} is independently selected from halo, OH, —NR^{N3}R^{N4}, —CN, —C(O)—R⁷, —SO₂—R⁷, C₁₋₃ alkoxy, C₁₋₃ haloalkoxy, C₃₋₆ cycloalkyl, 4 to 6 membered monocyclic heterocyclyl, and C₁₋₄ alkyl, wherein the C₁₋₄alkyl represented by R^{C1} is optionally substituted by one to three substituents independently selected from C₃₋₅ cycloalkyl, 4 to 8 membered monocyclic heterocyclyl, C₁₋₃alkoxy, OH, CN, —NR^{N3}R^{N4} and halo, and the C₁₋₃alkoxy represented by R^{C1} is optionally substituted with one to three substituents independently selected from C₁₋₃alkoxy and halo.

30. The compound of any one of claims 1-28, or a pharmaceutically acceptable salt thereof, wherein each R^{C1} is independently selected from halo, OH, —NR^{N3}R^{N4}, —CN, —C(O)—R⁷, —SO₂—R⁷, C₁₋₃ alkoxy, C₁₋₃ haloalkoxy, C₃₋₆ cycloalkyl, 4 to 6 membered monocyclic heterocyclyl, and C₁₋₄ alkyl, wherein the C₁₋₄alkyl represented by R^{C1} is optionally substituted by one to three substituents independently selected from C₃₋₅ cycloalkyl, C₁₋₃alkoxy, OH and halo, and the C₁₋₃alkoxy represented by R^{C1} is optionally substituted with one to three substituents independently selected from C₁₋₃alkoxy and halo.

31. The compound of claim 29, or a pharmaceutically acceptable salt thereof, wherein each R^{C1} is independently selected from F, —CN, —C(O)CH₃, —C(O)-cyclopropyl, —SO₂—CH₃, —SO₂—CH₂CH₃, OH, —OCH₃, —OCH₂CH₂OCH₃, —OCHF₂, —N(CH₃)₂, —CH₂CH₂N(CH₃)₂, —CH₃, —CH₂CH₃, —CH(CH₃)₂, —CH₂CH(CH₃)₂, —CH₂CH₂OH, —CH₂CH₂OCH₃, —CH₂OCH₃, —CH₂-cyclopropyl, —CH₂F, —CHF₂, —CF₃, —CH₂CF₃, —CF(CH₃)₂, —C(OH)(CH₃)₂, —CH₂CN, —CHFCH₃, —CF₂CH₂OCH₃, cyclobutyl, cyclopropyl,

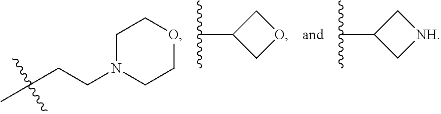

32. The compound of claim 30, or a pharmaceutically acceptable salt thereof, wherein each R^{C1} is independently selected from F, —CN, —C(O)CH₃, —C(O)-cyclopropyl, —SO₂—CH₃, —SO₂—CH₂CH₃, OH, —OCH₃, —OCH₂CH₂OCH₃, —OCHF₂, —N(CH₃)₂, —CH₃, —CH₂CH₃, —CH(CH₃)₂, —CH₂CH₂OH, —CH₂CH₂OCH₃, —CH₂-cyclopropyl, —CH₂F, —CHF₂, —CF₃, cyclobutyl, cyclopropyl,

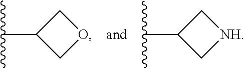 [SMILES: *C1COC1 ; *C1CNC1]

33. The compound of any one of claims 1-31, or a pharmaceutically acceptable salt thereof, wherein R¹ is C₁₋₄ alkyl optionally substituted by one to three R independently selected from halo, —CN, —NR²R⁴, C₁₋₃alkoxy, C₁₋₃alkyl and C₁₋₃haloalkyl, and R² and R⁴ are each independently H or C₁₋₃alkyl.

34. The compound of any one of claims 1-32, or a pharmaceutically acceptable salt thereof, wherein R¹ is C₁₋₄ alkyl optionally substituted by one to three R independently selected from halo, —CN, C₁₋₃alkoxy, C₁₋₃alkyl and C₁₋₃haloalkyl.

35. The compound of any one of claims 1-33, or a pharmaceutically acceptable salt thereof, wherein R¹ is —CH₃, —CH₂CH₃, —NH₂, —NHCH₃, —N(CH₃)₂, —OCH₃, or —CH₂CH₂OCH₃.

36. The compound of any one of claims 1-35, or a pharmaceutically acceptable salt thereof, wherein R¹ is —CH₃, —CH₂CH₃, or —CH₂CH₂OCH₃.

37. The compound of any one of claims 1-36, or a pharmaceutically acceptable salt thereof, wherein each R^{O1} is independently H, C₁₋₃ alkyl or C₃₋₆cycloalkyl, wherein the C₁₋₃ alkyl and C₃₋₆cycloalkyl represented by R^{O1} are optionally substituted by C₁₋₃ alkoxy.

38. The compound of claim 37, or a pharmaceutically acceptable salt thereof, wherein each R^{O1} is independently H, —CH₃, —CH₂—CH₂—OCH₃,

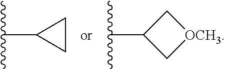

39. The compound of any one of claims 1-38, or a pharmaceutically acceptable salt thereof, wherein R^{N1} and R^{N2} each independently represent H or C₁₋₃ alkyl optionally substituted with C₁₋₃alkoxy.

40. The compound of claim 39, or a pharmaceutically acceptable salt thereof, wherein R^{N1} and R^{N2} each independently represent H, —CH₃ or —CH₂—CH₂—OCH₃.

41. The compound of claim 1, wherein the compound is represented by formula (X) or (XI): 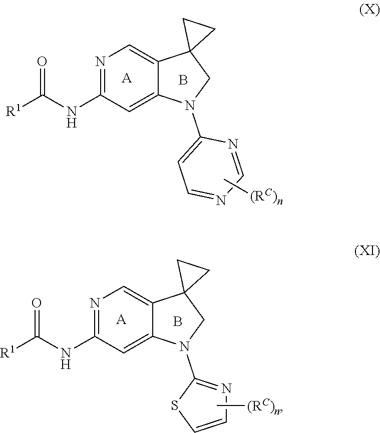
or a pharmaceutically acceptable salt thereof, wherein:
n is 1, 2 or 3;
each R^{C} is independently —NR^{N1}R^{N2}, —OR^{O1}, or C₁₋₆ alkyl, 4 to 6 membered monocyclic heterocyclyl, wherein the C₁₋₆ alkyl and 4 to 6 membered monocyclic heterocyclyl represented by R^{C} are each optionally substituted with one to three R^{C1};
each R^{C1} is independently halo;
R¹ is C₁₋₃ alkyl;
R^{O1} is C₃₋₄cycloalkyl optionally substituted by R^{O2};
R^{O2} is C₁₋₃ alkoxy; and
R^{N1} and R^{N2} are each independently H or C₁₋₃ alkyl optionally substituted with C₁₋₃ alkoxy.

42. The compound of claim 41, wherein the compound is represented by formula (XA), (XIA), (XIB), (XIC) or (XID): 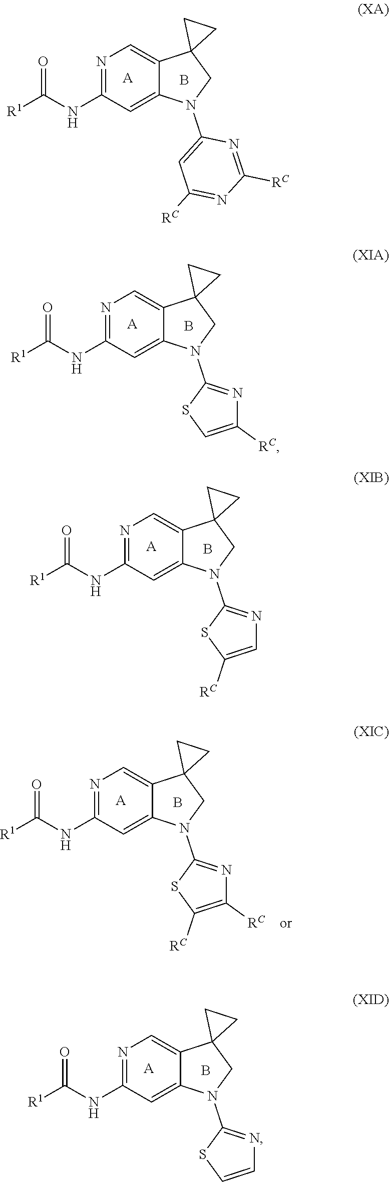
or a pharmaceutically acceptable salt thereof.

43. The compound of claim 41 or 42, or pharmaceutically acceptable salt thereof, wherein:
each R^{C} is independently —NHCH₃, —NHCH₂CH₂OCH₃, —CH₃, —CH₂CH₃, —CH(CH₃)₂, —CF₂CH₃, —CF(CH₃)₂, 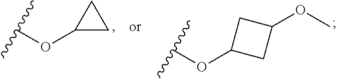
and
R¹ is CH₃.

44. The compound of claim 41 or 42, or a pharmaceutically acceptable salt thereof, wherein R^{C} is 4 to 6 membered monocyclic heterocyclyl independently selected from tetrahydrofuran and tetrahydropyran, each of which is optionally substituted with one to three R^{C1}.

45. The compound of claim 41 or 42, or a pharmaceutically acceptable salt thereof, wherein R^{C} is independently selected from 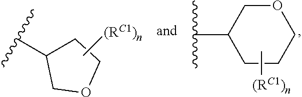
and wherein [symbol] represents a bond to ring C.

46. The compound of claim 41 or 42 or a pharmaceutically acceptable salt thereof, wherein R^{C} is independently selected from 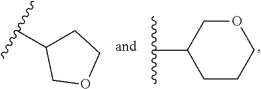 [SMILES: *C1CCOC1 ; *C1CCCOC1]
and wherein [symbol] represents a bond to ring C.

47. The compound of any one of claims 41 to 45, or a pharmaceutically acceptable salt thereof, wherein each R^{C1} is F.

48. The compound of claim 1, wherein the compound is represented by formula (XII): 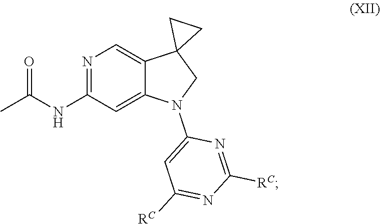
or a pharmaceutically acceptable salt thereof, wherein:
each R^{C} is independently C₁₋₃alkyl, C₁₋₃haloalkyl, or —NR^{N1}R^{N2};
R^{N1} and R^{N2} are each independently H or C₁₋₃alkyl.

49. The compound of claim 48, or a pharmaceutically acceptable salt thereof, wherein each R^{C} is, independently, —CH₃, —CH₂CH₃, —CF₂CH₃, or —NHCH₃.

50. A pharmaceutical composition comprising a compound according to any one of claims 1 to 49 or a pharmaceutically acceptable salt thereof, and a pharmaceutically acceptable carrier.

51. A method of inhibiting tyrosine kinase 2 (TYK2) activity in a subject in need thereof comprising administering to the subject an effective amount of a compound according to any one of claims 1 to 49 or a pharmaceutically acceptable salt thereof or a pharmaceutical composition according to claim 50.

52. A method of treating a disease or disorder responsive to inhibition of tyrosine kinase 2 (TYK2) in a subject comprising administering to the subject an effective amount of a compound according to any one of claims 1 to 49 or a pharmaceutically acceptable salt thereof or a pharmaceutical composition according to claim 50.

53. The method of claim 52, wherein the disease or disorder is inflammation, autoimmune disease, neuroinflammation, arthritis, rheumatoid arthritis, spondyloarthropathies, systemic lupus erythematosus, cutaneous lupus erythematosus, lupus nephritis, arthritis, osteoarthritis, gouty arthritis, pain, fever, pulmonary sarcoisosis, silicosis, cardiovascular disease, atherosclerosis, myocardial infarction, thrombosis, congestive heart failure and cardiac reperfusion injury, cardiomyopathy, stroke, ischaemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, systemic sclerosis, obesity, muscular dystrophy, polymyositis, dermatomyositis, autoimmune hepatitis, primary biliary cirrhosis, primary sclerosing cholangitis, vitiligo, alopecia, Alzheimer's disease, skin flushing, eczema, psoriasis, atopic dermatitis or sunburn.
